SEQUENCE LISTING (183 sequences)

SEQ ID NO: 1 - DNA, 16 bp, unidentified
  source 1..16: mol_type = unassigned DNA
gcgcgctcgctcgctc

SEQ ID NO: 2 - DNA, 16 bp, unidentified
  source 1..16: mol_type = unassigned DNA
gcgcgctcgctcgctc

SEQ ID NO: 3-7 - skipped (5 entries)

SEQ ID NO: 8 - DNA, 1923 bp, synthetic construct
  source 1..1923: mol_type = other DNA
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttttagccttcttctttttcctacagctcctgggcaacgtgctggttattgtgctgtctcatcatttgtcgacagaattcctcgaagatccgaaggggttcaagcttggcattccggtactgttggtaaagcca

SEQ ID NO: 9 - DNA, 16 bp, unidentified
  source 1..16: mol_type = unassigned DNA
gcgcgctcgctcgctc

SEQ ID NO: 10 - skipped

SEQ ID NO: 11 - protein, 1368 aa, Streptococcus pyogenes
MDKKYSIGLDIGTNSVGWAVITDEYKVTSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLYFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQDFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKEYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 12 - DNA, 4107 bp, synthetic construct
  source 1..4107: mol_type = other DNA
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 13 - DNA, 581 bp, synthetic construct
  source 1..581: mol_type = other DNA
gagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 14 - DNA, 225 bp, synthetic construct
  source 1..225: mol_type = other DNA
tgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggc

SEQ ID NO: 15 - DNA, 1272 bp, unidentified
  source 1..1272: mol_type = unassigned DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctggtctagaggtaccggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactga

SEQ ID NO: 16 - DNA, 1177 bp, unidentified
  source 1..1177: mol_type = unassigned DNA
ggctcagaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctggtctagaggtaccggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 17 - DNA, 547 bp, unidentified
  source 1..547: mol_type = unassigned DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactga

SEQ ID NO: 18 - DNA, 556 bp, unidentified
  source 1..556: mol_type = unassigned DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacactcgagggccctgtctcctcagcttcaggcaccaccactgacctgggacagtgaatccggacatcgattctaaggtaaatataaaatttttaagtgtataatttgttaaactactgattctaattgtttctctcttttagattccaacctttggaactga

SEQ ID NO: 19 - DNA, 1179 bp, Homo sapiens
  source 1..1179: mol_type = genomic DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 20 - DNA, 141 bp, Homo sapiens
  source 1..141: mol_type = genomic DNA
aataaacgataacgccgttggtggcgtgaggcatgtaaaaggttacatcattatcttgttcgccatccggttggtataaatagacgttcatgttggtttttgtttcagttgcaagttggctgcggcgcgcgcagcaccttt

SEQ ID NO: 21 - DNA, 317 bp, unidentified
  source 1..317: mol_type = unassigned DNA
ggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaa

SEQ ID NO: 22 - DNA, 241 bp, Homo sapiens
  source 1..241: mol_type = genomic DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 23 - DNA, 215 bp, Homo sapiens
  source 1..215: mol_type = genomic DNA
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcgtataagaactgtatgagaccac

SEQ ID NO: 24 - DNA, 546 bp, Homo sapiens
  source 1..546: mol_type = genomic DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactg

SEQ ID NO: 25 - protein, 19 aa, unidentified
MDWTWRILFLVAAATGAHS

SEQ ID NO: 26 - protein, 19 aa, unidentified
MLPSQLIGFLLLWVPASRG

SEQ ID NO: 27 - DNA, 2426 bp, synthetic construct
  source 1..2426: mol_type = other DNA
ctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatcgcggccgccaaactgttaatcctaacctaggttaagctacaccaaagagctgctgtcctgtggaattctgtagatcacagagacaaggcctgtgattgctgaacctgtatttgcatctgagaacccttaggagatctcttaggtcagtgaagagaagaacaaaaagcagcatattacagttagttgtcttcatcaatctttaaatatgttgtgtggtttttctctccctgtttccacagttgtttccaagggggaggagctgtttacaggtgtggtgcccatcctggttgaactggatggggatgtgaatggccacaagttctctgtttctggagaaggagagggagatgccacatatgggaagctcaccctgaaatttatctgcactactggcaagcttccagtgccttggcccacacttgtgactacattgacctatggagtccagtgtttcagcaggtaccctgaccacatgaagcagcatgacttttttaagagtgccatgcctgagggctatgtgcaggagagaaccatcttcttcaaggatgatggcaactacaagaccagagctgaggtgaagtttgaaggtgacacccttgtcaacaggatagaactaaagggtattgacttcaaagaagatggaaatattcttggccataagctggagtacaattacaactcccacaatgtctacatcatggctgacaagcagaagaatgggatcaaggtgaacttcaagatcaggcacaacattgaggatggatctgtgcagctggctgatcactaccagcagaacacacccattggtgatggtcctgtcctgctcccagacaaccactatttatccacccagtcagccctgtcaaaggacccaaatgagaagagggaccatatggtgctgttggagtttgtgacagctgcaggcatcactttgggcatggatgaactttacaaatgattaattaactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctaagggttctcagatgcaaataatatcaaagtgtccttattggatccttggctcctaaggttcacctcccagcacctggacagttgggcctgagatattgggttgtaagtggctggaaccatctagagcatggctacgtagataagtagcatggcgggttaatcattaactacacctgcaggaggaacccctagtgatggagttggccactccctctctgcgcgctcgctcgctcactgaggccgggcgaccaaaggtcgcccgacgcccgggctttgcccgggcggcctcagtgag

SEQ ID NO: 28 - DNA, 2426 bp, synthetic construct
  source 1..2426: mol_type = other DNA
ctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatcgcggccgccaaactgttaatcctaacctaggttaagctacaccaaagagctgctgtcctgtggaattctgtagatcacagagacaaggcctgtgattgctgaacctgtgttacaggaaaatctgaaggaggagatctcttaggtcagtgaagagaagaacaaaaagcagcatattacagttagttgtcttcatcaatctttaaatatgttgtgtggtttttctctccctgtttccacagttgtttccaagggggaggagctgtttacaggtgtggtgcccatcctggttgaactggatggggatgtgaatggccacaagttctctgtttctggagaaggagagggagatgccacatatgggaagctcaccctgaaatttatctgcactactggcaagcttccagtgccttggcccacacttgtgactacattgacctatggagtccagtgtttcagcaggtaccctgaccacatgaagcagcatgacttttttaagagtgccatgcctgagggctatgtgcaggagagaaccatcttcttcaaggatgatggcaactacaagaccagagctgaggtgaagtttgaaggtgacacccttgtcaacaggatagaactaaagggtattgacttcaaagaagatggaaatattcttggccataagctggagtacaattacaactcccacaatgtctacatcatggctgacaagcagaagaatgggatcaaggtgaacttcaagatcaggcacaacattgaggatggatctgtgcagctggctgatcactaccagcagaacacacccattggtgatggtcctgtcctgctcccagacaaccactatttatccacccagtcagccctgtcaaaggacccaaatgagaagagggaccatatggtgctgttggagtttgtgacagctgcaggcatcactttgggcatggatgaactttacaaatgattaattaactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctccttcagattttcctgtaacaatatcaaagtgtccttattggatccttggctcctaaggttcacctcccagcacctggacagttgggcctgagatattgggttgtaagtggctggaaccatctagagcatggctacgtagataagtagcatggcgggttaatcattaactacacctgcaggaggaacccctagtgatggagttggccactccctctctgcgcgctcgctcgctcactgaggccgggcgaccaaaggtcgcccgacgcccgggctttgcccgggcggcctcagtgag

SEQ ID NO: 29 - DNA, 2426 bp, synthetic construct
  source 1..2426: mol_type = other DNA
ctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatcgcggccgccaaactgttaatcctaacctaggttaagctacaccaaagagctgctgtcctgtggaattctgtagatcacagagacaaggcctgtgattgctgaacctgtcactcttgtctgtggaaacaaggagatctcttaggtcagtgaagagaagaacaaaaagcagcatattacagttagttgtcttcatcaatctttaaatatgttgtgtggtttttctctccctgtttccacagttgtttccaagggggaggagctgtttacaggtgtggtgcccatcctggttgaactggatggggatgtgaatggccacaagttctctgtttctggagaaggagagggagatgccacatatgggaagctcaccctgaaatttatctgcactactggcaagcttccagtgccttggcccacacttgtgactacattgacctatggagtccagtgtttcagcaggtaccctgaccacatgaagcagcatgacttttttaagagtgccatgcctgagggctatgtgcaggagagaaccatcttcttcaaggatgatggcaactacaagaccagagctgaggtgaagtttgaaggtgacacccttgtcaacaggatagaactaaagggtattgacttcaaagaagatggaaatattcttggccataagctggagtacaattacaactcccacaatgtctacatcatggctgacaagcagaagaatgggatcaaggtgaacttcaagatcaggcacaacattgaggatggatctgtgcagctggctgatcactaccagcagaacacacccattggtgatggtcctgtcctgctcccagacaaccactatttatccacccagtcagccctgtcaaaggacccaaatgagaagagggaccatatggtgctgttggagtttgtgacagctgcaggcatcactttgggcatggatgaactttacaaatgattaattaactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcccttgtttccacagacaagagtgaatatcaaagtgtccttattggatccttggctcctaaggttcacctcccagcacctggacagttgggcctgagatattgggttgtaagtggctggaaccatctagagcatggctacgtagataagtagcatggcgggttaatcattaactacacctgcaggaggaacccctagtgatggagttggccactccctctctgcgcgctcgctcgctcactgaggccgggcgaccaaaggtcgcccgacgcccgggctttgcccgggcggcctcagtgag

SEQ ID NO: 30 - DNA, 2426 bp, synthetic construct
  source 1..2426: mol_type = other DNA
ctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatcgcggccgccaaactgttaatcctaacctaggttaagctacaccaaagagctgctgtcctgtggaattctgtagatcacagagacaaggcctgtgattgctgaacctgttcactcttgtctgtggaaacaggagatctcttaggtcagtgaagagaagaacaaaaagcagcatattacagttagttgtcttcatcaatctttaaatatgttgtgtggtttttctctccctgtttccacagttgtttccaagggggaggagctgtttacaggtgtggtgcccatcctggttgaactggatggggatgtgaatggccacaagttctctgtttctggagaaggagagggagatgccacatatgggaagctcaccctgaaatttatctgcactactggcaagcttccagtgccttggcccacacttgtgactacattgacctatggagtccagtgtttcagcaggtaccctgaccacatgaagcagcatgacttttttaagagtgccatgcctgagggctatgtgcaggagagaaccatcttcttcaaggatgatggcaactacaagaccagagctgaggtgaagtttgaaggtgacacccttgtcaacaggatagaactaaagggtattgacttcaaagaagatggaaatattcttggccataagctggagtacaattacaactcccacaatgtctacatcatggctgacaagcagaagaatgggatcaaggtgaacttcaagatcaggcacaacattgaggatggatctgtgcagctggctgatcactaccagcagaacacacccattggtgatggtcctgtcctgctcccagacaaccactatttatccacccagtcagccctgtcaaaggacccaaatgagaagagggaccatatggtgctgttggagtttgtgacagctgcaggcatcactttgggcatggatgaactttacaaatgattaattaactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcccttgtttccacagacaagagtgaatatcaaagtgtccttattggatccttggctcctaaggttcacctcccagcacctggacagttgggcctgagatattgggttgtaagtggctggaaccatctagagcatggctacgtagataagtagcatggcgggttaatcattaactacacctgcaggaggaacccctagtgatggagttggccactccctctctgcgcgctcgctcgctcactgaggccgggcgaccaaaggtcgcccgacgcccgggctttgcccgggcggcctcagtgag

SEQ ID NO: 31 - DNA, 2180 bp, synthetic construct
  source 1..2180: mol_type = other DNA
ctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatcgcggccgcagatctcttaggtcagtgaagagaagaacaaaaagcagcatattacagttagttgtcttcatcaatctttaaatatgttgtgtggtttttctctccctgtttccacagttgtttccaagggggaggagctgtttacaggtgtggtgcccatcctggttgaactggatggggatgtgaatggccacaagttctctgtttctggagaaggagagggagatgccacatatgggaagctcaccctgaaatttatctgcactactggcaagcttccagtgccttggcccacacttgtgactacattgacctatggagtccagtgtttcagcaggtaccctgaccacatgaagcagcatgacttttttaagagtgccatgcctgagggctatgtgcaggagagaaccatcttcttcaaggatgatggcaactacaagaccagagctgaggtgaagtttgaaggtgacacccttgtcaacaggatagaactaaagggtattgacttcaaagaagatggaaatattcttggccataagctggagtacaattacaactcccacaatgtctacatcatggctgacaagcagaagaatgggatcaaggtgaacttcaagatcaggcacaacattgaggatggatctgtgcagctggctgatcactaccagcagaacacacccattggtgatggtcctgtcctgctcccagacaaccactatttatccacccagtcagccctgtcaaaggacccaaatgagaagagggaccatatggtgctgttggagtttgtgacagctgcaggcatcactttgggcatggatgaactttacaaatgattaattaactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgctctagagcatggctacgtagataagtagcatggcgggttaatcattaactacacctgcaggaggaacccctagtgatggagttggccactccctctctgcgcgctcgctcgctcactgaggccgggcgaccaaaggtcgcccgacgcccgggctttgcccgggcggcctcagtgag

SEQ ID NO: 32 - DNA, 5563 bp, synthetic construct
  source 1..5563: mol_type = other DNA
ctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatcgcggccgccaaactgttaatcctaacctaggttaagctacaccaaagagctgctgtcctgtggaattctgtagatcacagagacaaggcctgtgattgctgaacctgtcctaagggttctcagatgcaaatgtttaaacagatctcttaggtcagtgaagagaagaacaaaaagcagcatattacagttagttgtcttcatcaatctttaaatatgttgtgtggtttttctctccctgtttccacagttgaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaaaagcttgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccatatgtaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgaccaggttaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttaattaatcacacagcaatcttccctcctttctttgccttgatcaagatctccctgatttttctggcatccagttttccagtcaggcccttgggcacctcatctacaaagaccacccctcctctgagcttcttagctgtggtcacctggctggcaacatagtcaacaatctccttctcagtcatggtctttccatgttccaggacaaccacagcagctggaagttctccagcatcatcatctgggaggcctgccactcctgcatcaaagatgttggggtgctggagcaggatggactccagctcagcaggagccacttggtaccctttgtatttgatgagggacttcagcctgtccactatgaagaagtgctcatcttcatcccagtaggcaatgtctccactgtgcagccagccatctttgtcaatgagggcattggtggcctcagggttgttcacatatcctgacatgatcatggggcccctcacacacagctcacctctctgattcactcccagagtcttgcctgtgtccagatccaccaccttggcttcaaaaaatggcaccactttgcccactgctcctggcttgtcatctccttctggagtgatgagaatggcagaagttgtctctgtcaggccatagccctgtctgattccaggcaggtggaatcttttggccactgcctctcccacttccttggacagaggggctcctccagaagcaatttcatgcaggttgctcaggtcatacttatcaatcaaggtgctcttggcaaagaaggaaaagagggtgggcaccagcagagcagattggatcttgtagtcctgaaggctcctcaggaacagttcctcctcaaacctatacatcagcactactctgaatccacagatcaggtagcccagggtggtgaacatgccaaagccatgatggaaaggcacaacagacaggatggctgtatcagggatgatctggtttccaaagatggggtccctggcatgagagaacctgacacaggcagtcctgtggggcaaggccacacctttgggcagacctgtgctgccagagctgttcataatcagtgcaattgttttatctctgtcaaagctctctggaacaaaatcatactcattgaagccaggagggaggtggctggtgacaaaggtgtacatgctctggaaaccctggtaatctgtcttgctgtccatgataatgatcttctggatgattggcagcttcttctgcacattcagaattttctgcagcccctttttggacacaaacaccacagtgggctgggagatgcccatggagttgaggagctctctttcattgtagatgtcattggctggagcaacagctactccaatgaagagagctccaagcacaggcatgaagaactgcagggaattttcactgcacaccacaattctgtggttggtgttcagtccataccttttcatagcctctgccagtctcacagacatctcaaaatattcagcataggtgatatcaacttcaatgtgggcatcagtgaaggcaatggtgcctgggaccagggcatatctcttcatggccttgtgcagctgctcccctgctgttccatcctccagggggtagaagggggcagggcctttcttaatgttcttggcatcctcaactgtggaaacagggagagaaaaaccacacaacatatttaaagattgatgaagacaactaactgtaatatgctgctttttgttcttctcttcactgacctaagagatctgtcgacatttgcatctgagaacccttaggaatatcaaagtgtccttattggatccttggctcctaaggttcacctcccagcacctggacagttgggcctgagatattgggttgtaagtggctggaaccatctagagcatggctacgtagataagtagcatggcgggttaatcattaactacacctgcaggaggaacccctagtgatggagttggccactccctctctgcgcgctcgctcgctcactgaggccgggcgaccaaaggtcgcccgacgcccgggctttgcccgggcggcctcagtgag

SEQ ID NO: 33 - DNA, 5563 bp, synthetic construct
  source 1..5563: mol_type = other DNA
ctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatcgcggccgccaaactgttaatcctaacctaggttaagctacaccaaagagctgctgtcctgtggaattctgtagatcacagagacaaggcctgtgattgctgaacctgtatttgcatctgagaacccttagggtttaaacagatctcttaggtcagtgaagagaagaacaaaaagcagcatattacagttagttgtcttcatcaatctttaaatatgttgtgtggtttttctctccctgtttccacagttgaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaaaagcttgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccatatgtaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgaccaggttaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttaattaatcacacagcaatcttccctcctttctttgccttgatcaagatctccctgatttttctggcatccagttttccagtcaggcccttgggcacctcatctacaaagaccacccctcctctgagcttcttagctgtggtcacctggctggcaacatagtcaacaatctccttctcagtcatggtctttccatgttccaggacaaccacagcagctggaagttctccagcatcatcatctgggaggcctgccactcctgcatcaaagatgttggggtgctggagcaggatggactccagctcagcaggagccacttggtaccctttgtatttgatgagggacttcagcctgtccactatgaagaagtgctcatcttcatcccagtaggcaatgtctccactgtgcagccagccatctttgtcaatgagggcattggtggcctcagggttgttcacatatcctgacatgatcatggggcccctcacacacagctcacctctctgattcactcccagagtcttgcctgtgtccagatccaccaccttggcttcaaaaaatggcaccactttgcccactgctcctggcttgtcatctccttctggagtgatgagaatggcagaagttgtctctgtcaggccatagccctgtctgattccaggcaggtggaatcttttggccactgcctctcccacttccttggacagaggggctcctccagaagcaatttcatgcaggttgctcaggtcatacttatcaatcaaggtgctcttggcaaagaaggaaaagagggtgggcaccagcagagcagattggatcttgtagtcctgaaggctcctcaggaacagttcctcctcaaacctatacatcagcactactctgaatccacagatcaggtagcccagggtggtgaacatgccaaagccatgatggaaaggcacaacagacaggatggctgtatcagggatgatctggtttccaaagatggggtccctggcatgagagaacctgacacaggcagtcctgtggggcaaggccacacctttgggcagacctgtgctgccagagctgttcataatcagtgcaattgttttatctctgtcaaagctctctggaacaaaatcatactcattgaagccaggagggaggtggctggtgacaaaggtgtacatgctctggaaaccctggtaatctgtcttgctgtccatgataatgatcttctggatgattggcagcttcttctgcacattcagaattttctgcagcccctttttggacacaaacaccacagtgggctgggagatgcccatggagttgaggagctctctttcattgtagatgtcattggctggagcaacagctactccaatgaagagagctccaagcacaggcatgaagaactgcagggaattttcactgcacaccacaattctgtggttggtgttcagtccataccttttcatagcctctgccagtctcacagacatctcaaaatattcagcataggtgatatcaacttcaatgtgggcatcagtgaaggcaatggtgcctgggaccagggcatatctcttcatggccttgtgcagctgctcccctgctgttccatcctccagggggtagaagggggcagggcctttcttaatgttcttggcatcctcaactgtggaaacagggagagaaaaaccacacaacatatttaaagattgatgaagacaactaactgtaatatgctgctttttgttcttctcttcactgacctaagagatctgtcgaccctaagggttctcagatgcaaataatatcaaagtgtccttattggatccttggctcctaaggttcacctcccagcacctggacagttgggcctgagatattgggttgtaagtggctggaaccatctagagcatggctacgtagataagtagcatggcgggttaatcattaactacacctgcaggaggaacccctagtgatggagttggccactccctctctgcgcgctcgctcgctcactgaggccgggcgaccaaaggtcgcccgacgcccgggctttgcccgggcggcctcagtgag

SEQ ID NO: 34 - DNA, 5563 bp, synthetic construct
  source 1..5563: mol_type = other DNA
ctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatcgcggccgccaaactgttaatcctaacctaggttaagctacaccaaagagctgctgtcctgtggaattctgtagatcacagagacaaggcctgtgattgctgaacctgtcctccttcagattttcctgtaacgtttaaacagatctcttaggtcagtgaagagaagaacaaaaagcagcatattacagttagttgtcttcatcaatctttaaatatgttgtgtggtttttctctccctgtttccacagttgaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaaaagcttgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccatatgtaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgaccaggttaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttaattaatcacacagcaatcttccctcctttctttgccttgatcaagatctccctgatttttctggcatccagttttccagtcaggcccttgggcacctcatctacaaagaccacccctcctctgagcttcttagctgtggtcacctggctggcaacatagtcaacaatctccttctcagtcatggtctttccatgttccaggacaaccacagcagctggaagttctccagcatcatcatctgggaggcctgccactcctgcatcaaagatgttggggtgctggagcaggatggactccagctcagcaggagccacttggtaccctttgtatttgatgagggacttcagcctgtccactatgaagaagtgctcatcttcatcccagtaggcaatgtctccactgtgcagccagccatctttgtcaatgagggcattggtggcctcagggttgttcacatatcctgacatgatcatggggcccctcacacacagctcacctctctgattcactcccagagtcttgcctgtgtccagatccaccaccttggcttcaaaaaatggcaccactttgcccactgctcctggcttgtcatctccttctggagtgatgagaatggcagaagttgtctctgtcaggccatagccctgtctgattccaggcaggtggaatcttttggccactgcctctcccacttccttggacagaggggctcctccagaagcaatttcatgcaggttgctcaggtcatacttatcaatcaaggtgctcttggcaaagaaggaaaagagggtgggcaccagcagagcagattggatcttgtagtcctgaaggctcctcaggaacagttcctcctcaaacctatacatcagcactactctgaatccacagatcaggtagcccagggtggtgaacatgccaaagccatgatggaaaggcacaacagacaggatggctgtatcagggatgatctggtttccaaagatggggtccctggcatgagagaacctgacacaggcagtcctgtggggcaaggccacacctttgggcagacctgtgctgccagagctgttcataatcagtgcaattgttttatctctgtcaaagctctctggaacaaaatcatactcattgaagccaggagggaggtggctggtgacaaaggtgtacatgctctggaaaccctggtaatctgtcttgctgtccatgataatgatcttctggatgattggcagcttcttctgcacattcagaattttctgcagcccctttttggacacaaacaccacagtgggctgggagatgcccatggagttgaggagctctctttcattgtagatgtcattggctggagcaacagctactccaatgaagagagctccaagcacaggcatgaagaactgcagggaattttcactgcacaccacaattctgtggttggtgttcagtccataccttttcatagcctctgccagtctcacagacatctcaaaatattcagcataggtgatatcaacttcaatgtgggcatcagtgaaggcaatggtgcctgggaccagggcatatctcttcatggccttgtgcagctgctcccctgctgttccatcctccagggggtagaagggggcagggcctttcttaatgttcttggcatcctcaactgtggaaacagggagagaaaaaccacacaacatatttaaagattgatgaagacaactaactgtaatatgctgctttttgttcttctcttcactgacctaagagatctgtcgacgttacaggaaaatctgaaggaggaatatcaaagtgtccttattggatccttggctcctaaggttcacctcccagcacctggacagttgggcctgagatattgggttgtaagtggctggaaccatctagagcatggctacgtagataagtagcatggcgggttaatcattaactacacctgcaggaggaacccctagtgatggagttggccactccctctctgcgcgctcgctcgctcactgaggccgggcgaccaaaggtcgcccgacgcccgggctttgcccgggcggcctcagtgag

SEQ ID NO: 35 - DNA, 5563 bp, synthetic construct
  source 1..5563: mol_type = other DNA
ctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatcgcggccgccaaactgttaatcctaacctaggttaagctacaccaaagagctgctgtcctgtggaattctgtagatcacagagacaaggcctgtgattgctgaacctgtgttacaggaaaatctgaaggagggtttaaacagatctcttaggtcagtgaagagaagaacaaaaagcagcatattacagttagttgtcttcatcaatctttaaatatgttgtgtggtttttctctccctgtttccacagttgaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaaaagcttgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccatatgtaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgaccaggttaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttaattaatcacacagcaatcttccctcctttctttgccttgatcaagatctccctgatttttctggcatccagttttccagtcaggcccttgggcacctcatctacaaagaccacccctcctctgagcttcttagctgtggtcacctggctggcaacatagtcaacaatctccttctcagtcatggtctttccatgttccaggacaaccacagcagctggaagttctccagcatcatcatctgggaggcctgccactcctgcatcaaagatgttggggtgctggagcaggatggactccagctcagcaggagccacttggtaccctttgtatttgatgagggacttcagcctgtccactatgaagaagtgctcatcttcatcccagtaggcaatgtctccactgtgcagccagccatctttgtcaatgagggcattggtggcctcagggttgttcacatatcctgacatgatcatggggcccctcacacacagctcacctctctgattcactcccagagtcttgcctgtgtccagatccaccaccttggcttcaaaaaatggcaccactttgcccactgctcctggcttgtcatctccttctggagtgatgagaatggcagaagttgtctctgtcaggccatagccctgtctgattccaggcaggtggaatcttttggccactgcctctcccacttccttggacagaggggctcctccagaagcaatttcatgcaggttgctcaggtcatacttatcaatcaaggtgctcttggcaaagaaggaaaagagggtgggcaccagcagagcagattggatcttgtagtcctgaaggctcctcaggaacagttcctcctcaaacctatacatcagcactactctgaatccacagatcaggtagcccagggtggtgaacatgccaaagccatgatggaaaggcacaacagacaggatggctgtatcagggatgatctggtttccaaagatggggtccctggcatgagagaacctgacacaggcagtcctgtggggcaaggccacacctttgggcagacctgtgctgccagagctgttcataatcagtgcaattgttttatctctgtcaaagctctctggaacaaaatcatactcattgaagccaggagggaggtggctggtgacaaaggtgtacatgctctggaaaccctggtaatctgtcttgctgtccatgataatgatcttctggatgattggcagcttcttctgcacattcagaattttctgcagcccctttttggacacaaacaccacagtgggctgggagatgcccatggagttgaggagctctctttcattgtagatgtcattggctggagcaacagctactccaatgaagagagctccaagcacaggcatgaagaactgcagggaattttcactgcacaccacaattctgtggttggtgttcagtccataccttttcatagcctctgccagtctcacagacatctcaaaatattcagcataggtgatatcaacttcaatgtgggcatcagtgaaggcaatggtgcctgggaccagggcatatctcttcatggccttgtgcagctgctcccctgctgttccatcctccagggggtagaagggggcagggcctttcttaatgttcttggcatcctcaactgtggaaacagggagagaaaaaccacacaacatatttaaagattgatgaagacaactaactgtaatatgctgctttttgttcttctcttcactgacctaagagatctgtcgaccctccttcagattttcctgtaacaatatcaaagtgtccttattggatccttggctcctaaggttcacctcccagcacctggacagttgggcctgagatattgggttgtaagtggctggaaccatctagagcatggctacgtagataagtagcatggcgggttaatcattaactacacctgcaggaggaacccctagtgatggagttggccactccctctctgcgcgctcgctcgctcactgaggccgggcgaccaaaggtcgcccgacgcccgggctttgcccgggcggcctcagtgag

SEQ ID NO: 36 - DNA, 5317 bp, synthetic construct
  source 1..5317: mol_type = other DNA
ctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatcgcggccgcgtttaaacagatctcttaggtcagtgaagagaagaacaaaaagcagcatattacagttagttgtcttcatcaatctttaaatatgttgtgtggtttttctctccctgtttccacagttgaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaaaagcttgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggccatatgtaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgaccaggttaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttaattaatcacacagcaatcttccctcctttctttgccttgatcaagatctccctgatttttctggcatccagttttccagtcaggcccttgggcacctcatctacaaagaccacccctcctctgagcttcttagctgtggtcacctggctggcaacatagtcaacaatctccttctcagtcatggtctttccatgttccaggacaaccacagcagctggaagttctccagcatcatcatctgggaggcctgccactcctgcatcaaagatgttggggtgctggagcaggatggactccagctcagcaggagccacttggtaccctttgtatttgatgagggacttcagcctgtccactatgaagaagtgctcatcttcatcccagtaggcaatgtctccactgtgcagccagccatctttgtcaatgagggcattggtggcctcagggttgttcacatatcctgacatgatcatggggcccctcacacacagctcacctctctgattcactcccagagtcttgcctgtgtccagatccaccaccttggcttcaaaaaatggcaccactttgcccactgctcctggcttgtcatctccttctggagtgatgagaatggcagaagttgtctctgtcaggccatagccctgtctgattccaggcaggtggaatcttttggccactgcctctcccacttccttggacagaggggctcctccagaagcaatttcatgcaggttgctcaggtcatacttatcaatcaaggtgctcttggcaaagaaggaaaagagggtgggcaccagcagagcagattggatcttgtagtcctgaaggctcctcaggaacagttcctcctcaaacctatacatcagcactactctgaatccacagatcaggtagcccagggtggtgaacatgccaaagccatgatggaaaggcacaacagacaggatggctgtatcagggatgatctggtttccaaagatggggtccctggcatgagagaacctgacacaggcagtcctgtggggcaaggccacacctttgggcagacctgtgctgccagagctgttcataatcagtgcaattgttttatctctgtcaaagctctctggaacaaaatcatactcattgaagccaggagggaggtggctggtgacaaaggtgtacatgctctggaaaccctggtaatctgtcttgctgtccatgataatgatcttctggatgattggcagcttcttctgcacattcagaattttctgcagcccctttttggacacaaacaccacagtgggctgggagatgcccatggagttgaggagctctctttcattgtagatgtcattggctggagcaacagctactccaatgaagagagctccaagcacaggcatgaagaactgcagggaattttcactgcacaccacaattctgtggttggtgttcagtccataccttttcatagcctctgccagtctcacagacatctcaaaatattcagcataggtgatatcaacttcaatgtgggcatcagtgaaggcaatggtgcctgggaccagggcatatctcttcatggccttgtgcagctgctcccctgctgttccatcctccagggggtagaagggggcagggcctttcttaatgttcttggcatcctcaactgtggaaacagggagagaaaaaccacacaacatatttaaagattgatgaagacaactaactgtaatatgctgctttttgttcttctcttcactgacctaagagatctgtcgactctagagcatggctacgtagataagtagcatggcgggttaatcattaactacacctgcaggaggaacccctagtgatggagttggccactccctctctgcgcgctcgctcgctcactgaggccgggcgaccaaaggtcgcccgacgcccgggctttgcccgggcggcctcagtgag

SEQ ID NO: 37 - DNA, 3625 bp, synthetic construct
  source 1..3625: mol_type = other DNA
ctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatcgcggccgctctagaagatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttctatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgttaggtgagcttagtcttttcttttatccaattcacgtagcgagacaccttcgtatagatgccatatttccccttcatcgcacattcctccccccaacttattatcccggtcaagaaacttgttccttcgacttcagtgacgtgtggtccacctgaatcaccttggcatgagtcgcgaccgccctcgtgaaacccagcacaaaacatgttattgtaaatcgtaaatttcgtggacagaagacaggtcgctctatcgaccaacgggacgcgcaaatattgcagaacgagggctgatcgacctttgtggaagacccgcccccacccactcacatatccgctcccaaatttcaagaagatatttgtatattctttatcggctatacaaatcggggtaacataggagttaagtacgagtggctcgtccagctccaggagggctatatcatggttgtacttgtttatagcggcattataattgtgatggggtatgatcctgataacattccttttctgttcagtatgctcagtttcttcaatgttgtgttcgccagccacgaccgtaatcttaaccccagtctcgacacagtgtgcggccgttacaatccacttttcattgactatggagcccccacaaaacgcgtcgacttttccgttgagcaccacctgccatggaaattggccaggtttagcgtcctcgcccccgacaaccctagtaaagtcattaaatgactgtgtggattgtgttatattatcaagaatcgtttcggcttcagtagagttaacgtagtccacatcgggaaaaactgtctcggcccttgtcaactttgatgtctgggacacacttacccgaccgcacgggaagggcaccgccggttcacagctcttttgattctcagcgagccggtagccctcagtgcaactacacacaactttgttgtcggcggaatttttacagaattgctcgcatcgtccatttttaatgttgcaggtgacgtccaactcgcagttttttccttcaaaaccaaaagggcaccaacactcgtaggaatttatatcgtctttacaactccccccattcagacatggattagattcgcattggtccccatcgacatattgcttccagaactcagtggtccgttctgtattctcaaacacctcgcgcgcttcttcaaaactgcatttttcctccatacactctcgctccaagttcccttgcacgaattcttcaagctttcctgagttataccttttaggccggttaagtatcttattcgcgttttcgtggtccagaaaaactgtggaaacagggagagaaaaaccacacaacatatttaaagattgatgaagacaactaactgtaatatgctgctttttgttcttctcttcactgacctaagagatctggatcctctagagcatggctacgtagataagtagcatggcgggttaatcattaactacacctgcaggaggaacccctagtgatggagttggccactccctctctgcgcgctcgctcgctcactgaggccgggcgaccaaaggtcgcccgacgcccgggctttgcccgggcggcctcagtgag

SEQ ID NO: 38 - DNA, 3883 bp, synthetic construct
  source 1..3883: mol_type = other DNA
ctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatcgcggccgccaaactgttaatcctaacctaggttaagctacaccaaagagctgctgtcctgtggaattctgtagatcacagagacaaggcctgtgattgctgaacctgtatttgcatctgagaacccttaggggtacctctagaagatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttctatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgttaggtgagcttagtcttttcttttatccaattcacgtagcgagacaccttcgtatagatgccatatttccccttcatcgcacattcctccccccaacttattatcccggtcaagaaacttgttccttcgacttcagtgacgtgtggtccacctgaatcaccttggcatgagtcgcgaccgccctcgtgaaacccagcacaaaacatgttattgtaaatcgtaaatttcgtggacagaagacaggtcgctctatcgaccaacgggacgcgcaaatattgcagaacgagggctgatcgacctttgtggaagacccgcccccacccactcacatatccgctcccaaatttcaagaagatatttgtatattctttatcggctatacaaatcggggtaacataggagttaagtacgagtggctcgtccagctccaggagggctatatcatggttgtacttgtttatagcggcattataattgtgatggggtatgatcctgataacattccttttctgttcagtatgctcagtttcttcaatgttgtgttcgccagccacgaccgtaatcttaaccccagtctcgacacagtgtgcggccgttacaatccacttttcattgactatggagcccccacaaaacgcgtcgacttttccgttgagcaccacctgccatggaaattggccaggtttagcgtcctcgcccccgacaaccctagtaaagtcattaaatgactgtgtggattgtgttatattatcaagaatcgtttcggcttcagtagagttaacgtagtccacatcgggaaaaactgtctcggcccttgtcaactttgatgtctgggacacacttacccgaccgcacgggaagggcaccgccggttcacagctcttttgattctcagcgagccggtagccctcagtgcaactacacacaactttgttgtcggcggaatttttacagaattgctcgcatcgtccatttttaatgttgcaggtgacgtccaactcgcagttttttccttcaaaaccaaaagggcaccaacactcgtaggaatttatatcgtctttacaactccccccattcagacatggattagattcgcattggtccccatcgacatattgcttccagaactcagtggtccgttctgtattctcaaacacctcgcgcgcttcttcaaaactgcatttttcctccatacactctcgctccaagttcccttgcacgaattcttcaagctttcctgagttataccttttaggccggttaagtatcttattcgcgttttcgtggtccagaaaaactgtggaaacagggagagaaaaaccacacaacatatttaaagattgatgaagacaactaactgtaatatgctgctttttgttcttctcttcactgacctaagagatctggatccgagctccctaagggttctcagatgcaaataatatcaaagtgtccttattggatccttggctcctaaggttcacctcccagcacctggacagttgggcctgagatattgggttgtaagtggctggaaccatctagagcatggctacgtagataagtagcatggcgggttaatcattaactacacctgcaggaggaacccctagtgatggagttggccactccctctctgcgcgctcgctcgctcactgaggccgggcgaccaaaggtcgcccgacgcccgggctttgcccgggcggcctcagtgag

SEQ ID NO: 39 - DNA, 3629 bp, synthetic construct
  source 1..3629: mol_type = other DNA
ctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatcgcggccgcagatctcttaggtcagtgaagagaagaacaaaaagcagcatattacagttagttgtcttcatcaatctttaaatatgttgtgtggtttttctctccctgtttccacagttgtttccaagggggaggagctgtttacaggtgtggtgcccatcctggttgaactggatggggatgtgaatggccacaagttctctgtttctggagaaggagagggagatgccacatatgggaagctcaccctgaaatttatctgcactactggcaagcttccagtgccttggcccacacttgtgactacattgacctatggagtccagtgtttcagcaggtaccctgaccacatgaagcagcatgacttttttaagagtgccatgcctgagggctatgtgcaggagagaaccatcttcttcaaggatgatggcaactacaagaccagagctgaggtgaagtttgaaggtgacacccttgtcaacaggatagaactaaagggtattgacttcaaagaagatggaaatattcttggccataagctggagtacaattacaactcccacaatgtctacatcatggctgacaagcagaagaatgggatcaaggtgaacttcaagatcaggcacaacattgaggatggatctgtgcagctggctgatcactaccagcagaacacacccattggtgatggtcctgtcctgctcccagacaaccactatttatccacccagtcagccctgtcaaaggacccaaatgagaagagggaccatatggtgctgttggagtttgtgacagctgcaggcatcactttgggcatggatgaactttacaaatgattaattaactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgctaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttaattaactactacttgtacagctcgtccatgccgccggtggagtggcggccctcggcgcgttcgtactgttccacgatggtgtagtcctcgttgtgggaggtgatgtccaacttgatgttgacgttgtaggcgccgggcagctgcacgggcttcttggccttgtaggtggtcttgacctcagcgtcgtagtggccgccgtccttcagcttcagcctctgcttgatctcgcccttcagggcgccgtcctcggggtacatccgctcggaggaggcctcccagcccatggtcttcttctgcattacggggccgtcggaggggaagttggtgccgcgcagcttcaccttgtagatgaactcgccgtcctgcagggaggagtcctgggtcacggtcaccacgccgccgtcctcgaagttcatcacgcgctcccacttgaagccctcggggaaggacagcttcaagtagtcggggatgtcggcggggtgcttcacgtaggccttggagccgtacatgaactgaggggacaggatgtcccaggcgaagggcagggggccacccttggtcaccttcagcttggcggtctgggtgccctcgtaggggcggccctcgccctcgccctcgatctcgaactcgtggccgttcacggagccctccatgtgcaccttgaagcgcatgaactccttgatgatggccatgttatcctcctcgcccttgctcacaactgtggaaacagggagagaaaaaccacacaacatatttaaagattgatgaagacaactaactgtaatatgctgctttttgttcttctcttcactgacctaagagatctccttctagagcatggctacgtagataagtagcatggcgggttaatcattaactacacctgcaggaggaacccctagtgatggagttggccactccctctctgcgcgctcgctcgctcactgaggccgggcgaccaaaggtcgcccgacgcccgggctttgcccgggcggcctcagtgag

SEQ ID NO: 40 - DNA, 3872 bp, synthetic construct
  source 1..3872: mol_type = other DNA
ctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatcgcggccgccaaactgttaatcctaacctaggttaagctacaccaaagagctgctgtcctgtggaattctgtagatcacagagacaaggcctgtgattgctgaacctgtatttgcatctgagaacccttaggagatctcttaggtcagtgaagagaagaacaaaaagcagcatattacagttagttgtcttcatcaatctttaaatatgttgtgtggtttttctctccctgtttccacagttgtttccaagggggaggagctgtttacaggtgtggtgcccatcctggttgaactggatggggatgtgaatggccacaagttctctgtttctggagaaggagagggagatgccacatatgggaagctcaccctgaaatttatctgcactactggcaagcttccagtgccttggcccacacttgtgactacattgacctatggagtccagtgtttcagcaggtaccctgaccacatgaagcagcatgacttttttaagagtgccatgcctgagggctatgtgcaggagagaaccatcttcttcaaggatgatggcaactacaagaccagagctgaggtgaagtttgaaggtgacacccttgtcaacaggatagaactaaagggtattgacttcaaagaagatggaaatattcttggccataagctggagtacaattacaactcccacaatgtctacatcatggctgacaagcagaagaatgggatcaaggtgaacttcaagatcaggcacaacattgaggatggatctgtgcagctggctgatcactaccagcagaacacacccattggtgatggtcctgtcctgctcccagacaaccactatttatccacccagtcagccctgtcaaaggacccaaatgagaagagggaccatatggtgctgttggagtttgtgacagctgcaggcatcactttgggcatggatgaactttacaaatgattaattaactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgctaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttaattaactactacttgtacagctcgtccatgccgccggtggagtggcggccctcggcgcgttcgtactgttccacgatggtgtagtcctcgttgtgggaggtgatgtccaacttgatgttgacgttgtaggcgccgggcagctgcacgggcttcttggccttgtaggtggtcttgacctcagcgtcgtagtggccgccgtccttcagcttcagcctctgcttgatctcgcccttcagggcgccgtcctcggggtacatccgctcggaggaggcctcccagcccatggtcttcttctgcattacggggccgtcggaggggaagttggtgccgcgcagcttcaccttgtagatgaactcgccgtcctgcagggaggagtcctgggtcacggtcaccacgccgccgtcctcgaagttcatcacgcgctcccacttgaagccctcggggaaggacagcttcaagtagtcggggatgtcggcggggtgcttcacgtaggccttggagccgtacatgaactgaggggacaggatgtcccaggcgaagggcagggggccacccttggtcaccttcagcttggcggtctgggtgccctcgtaggggcggccctcgccctcgccctcgatctcgaactcgtggccgttcacggagccctccatgtgcaccttgaagcgcatgaactccttgatgatggccatgttatcctcctcgcccttgctcacaactgtggaaacagggagagaaaaaccacacaacatatttaaagattgatgaagacaactaactgtaatatgctgctttttgttcttctcttcactgacctaagagatctcctaagggttctcagatgcaaataatatcaaagtgtccttattggatccttggctcctaaggttcacctcccagcacctggacagttgggcctgagatattgggttgtaagtggctggaaccatctagagcatggctacgtagataagtagcatggcgggttaatcattaactacacctgcaggaggaacccctagtgatggagttggccactccctctctgcgcgctcgctcgctcactgaggccgggcgaccaaaggtcgcccgacgcccgggctttgcccgggcggcctcagtgag

SEQ ID NO: 41 - DNA, 3875 bp, synthetic construct
  source 1..3875: mol_type = other DNA
ctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatcgcggccgccaaactgttaatcctaacctaggttaagctacaccaaagagctgctgtcctgtggaattctgtagatcacagagacaaggcctgtgattgctgaacctgtgttacaggaaaatctgaaggaggagatctcttaggtcagtgaagagaagaacaaaaagcagcatattacagttagttgtcttcatcaatctttaaatatgttgtgtggtttttctctccctgtttccacagttgtttccaagggggaggagctgtttacaggtgtggtgcccatcctggttgaactggatggggatgtgaatggccacaagttctctgtttctggagaaggagagggagatgccacatatgggaagctcaccctgaaatttatctgcactactggcaagcttccagtgccttggcccacacttgtgactacattgacctatggagtccagtgtttcagcaggtaccctgaccacatgaagcagcatgacttttttaagagtgccatgcctgagggctatgtgcaggagagaaccatcttcttcaaggatgatggcaactacaagaccagagctgaggtgaagtttgaaggtgacacccttgtcaacaggatagaactaaagggtattgacttcaaagaagatggaaatattcttggccataagctggagtacaattacaactcccacaatgtctacatcatggctgacaagcagaagaatgggatcaaggtgaacttcaagatcaggcacaacattgaggatggatctgtgcagctggctgatcactaccagcagaacacacccattggtgatggtcctgtcctgctcccagacaaccactatttatccacccagtcagccctgtcaaaggacccaaatgagaagagggaccatatggtgctgttggagtttgtgacagctgcaggcatcactttgggcatggatgaactttacaaatgattaattaactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgctaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttaattaactactacttgtacagctcgtccatgccgccggtggagtggcggccctcggcgcgttcgtactgttccacgatggtgtagtcctcgttgtgggaggtgatgtccaacttgatgttgacgttgtaggcgccgggcagctgcacgggcttcttggccttgtaggtggtcttgacctcagcgtcgtagtggccgccgtccttcagcttcagcctctgcttgatctcgcccttcagggcgccgtcctcggggtacatccgctcggaggaggcctcccagcccatggtcttcttctgcattacggggccgtcggaggggaagttggtgccgcgcagcttcaccttgtagatgaactcgccgtcctgcagggaggagtcctgggtcacggtcaccacgccgccgtcctcgaagttcatcacgcgctcccacttgaagccctcggggaaggacagcttcaagtagtcggggatgtcggcggggtgcttcacgtaggccttggagccgtacatgaactgaggggacaggatgtcccaggcgaagggcagggggccacccttggtcaccttcagcttggcggtctgggtgccctcgtaggggcggccctcgccctcgccctcgatctcgaactcgtggccgttcacggagccctccatgtgcaccttgaagcgcatgaactccttgatgatggccatgttatcctcctcgcccttgctcacaactgtggaaacagggagagaaaaaccacacaacatatttaaagattgatgaagacaactaactgtaatatgctgctttttgttcttctcttcactgacctaagagatctcctcctccttcagattttcctgtaacaatatcaaagtgtccttattggatccttggctcctaaggttcacctcccagcacctggacagttgggcctgagatattgggttgtaagtggctggaaccatctagagcatggctacgtagataagtagcatggcgggttaatcattaactacacctgcaggaggaacccctagtgatggagttggccactccctctctgcgcgctcgctcgctcactgaggccgggcgaccaaaggtcgcccgacgcccgggctttgcccgggcggcctcagtgag

SEQ ID NO: 42 - DNA, 3873 bp, synthetic construct
  source 1..3873: mol_type = other DNA
ctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatcgcggccgccaaactgttaatcctaacctaggttaagctacaccaaagagctgctgtcctgtggaattctgtagatcacagagacaaggcctgtgattgctgaacctgtcactcttgtctgtggaaacaaggagatctcttaggtcagtgaagagaagaacaaaaagcagcatattacagttagttgtcttcatcaatctttaaatatgttgtgtggtttttctctccctgtttccacagttgtttccaagggggaggagctgtttacaggtgtggtgcccatcctggttgaactggatggggatgtgaatggccacaagttctctgtttctggagaaggagagggagatgccacatatgggaagctcaccctgaaatttatctgcactactggcaagcttccagtgccttggcccacacttgtgactacattgacctatggagtccagtgtttcagcaggtaccctgaccacatgaagcagcatgacttttttaagagtgccatgcctgagggctatgtgcaggagagaaccatcttcttcaaggatgatggcaactacaagaccagagctgaggtgaagtttgaaggtgacacccttgtcaacaggatagaactaaagggtattgacttcaaagaagatggaaatattcttggccataagctggagtacaattacaactcccacaatgtctacatcatggctgacaagcagaagaatgggatcaaggtgaacttcaagatcaggcacaacattgaggatggatctgtgcagctggctgatcactaccagcagaacacacccattggtgatggtcctgtcctgctcccagacaaccactatttatccacccagtcagccctgtcaaaggacccaaatgagaagagggaccatatggtgctgttggagtttgtgacagctgcaggcatcactttgggcatggatgaactttacaaatgattaattaactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttatctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgctaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttaattaactactacttgtacagctcgtccatgccgccggtggagtggcggccctcggcgcgttcgtactgttccacgatggtgtagtcctcgttgtgggaggtgatgtccaacttgatgttgacgttgtaggcgccgggcagctgcacgggcttcttggccttgtaggtggtcttgacctcagcgtcgtagtggccgccgtccttcagcttcagcctctgcttgatctcgcccttcagggcgccgtcctcggggtacatccgctcggaggaggcctcccagcccatggtcttcttctgcattacggggccgtcggaggggaagttggtgccgcgcagcttcaccttgtagatgaactcgccgtcctgcagggaggagtcctgggtcacggtcaccacgccgccgtcctcgaagttcatcacgcgctcccacttgaagccctcggggaaggacagcttcaagtagtcggggatgtcggcggggtgcttcacgtaggccttggagccgtacatgaactgaggggacaggatgtcccaggcgaagggcagggggccacccttggtcaccttcagcttggcggtctgggtgccctcgtaggggcggccctcgccctcgccctcgatctcgaactcgtggccgttcacggagccctccatgtgcaccttgaagcgcatgaactccttgatgatggccatgttatcctcctcgcccttgctcacaactgtggaaacagggagagaaaaaccacacaacatatttaaagattgatgaagacaactaactgtaatatgctgctttttgttcttctcttcactgacctaagagatctccttgtttccacagacaagagtgaatatcaaagtgtccttattggatccttggctcctaaggttcacctcccagcacctggacagttgggcctgagatattgggttgtaagtggctggaaccatctagagcatggctacgtagataagtagcatggcgggttaatcattaactacacctgcaggaggaacccctagtgatggagttggccactccctctctgcgcgctcgctcgctcactgaggccgggcgaccaaaggtcgcccgacgcccgggctttgcccgggcggcctcagtgag

SEQ ID NO: 43 - DNA, 2226 bp, synthetic construct
  source 1..2226: mol_type = other DNA
ctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatcgcggccgccactcttgtctgtggaaacaagagatctcttaggtcagtgaagagaagaacaaaaagcagcatattacagttagttgtcttcatcaatctttaaatatgttgtgtggtttttctctccctgtttccacagttggtttccaagggggaggagctgtttacaggtgtggtgcccatcctggttgaactggatggggatgtgaatggccacaagttctctgtttctggagaaggagagggagatgccacatatgggaagctcaccctgaaatttatctgcactactggcaagcttccagtgccttggcccacacttgtgactacattgacctatggagtccagtgtttcagcaggtaccctgaccacatgaagcagcatgacttttttaagagtgccatgcctgagggctatgtgcaggagagaaccatcttcttcaaggatgatggcaactacaagaccagagctgaggtgaagtttgaaggtgacacccttgtcaacaggatagaactaaagggtattgacttcaaagaagatggaaatattcttggccataagctggagtacaattacaactcccacaatgtctacatcatggctgacaagcagaagaatgggatcaaggtgaacttcaagatcaggcacaacattgaggatggatctgtgcagctggctgatcactaccagcagaacacacccattggtgatggtcctgtcctgctcccagacaaccactatttatccacccagtcagccctgtcaaaggacccaaatgagaagagggaccatatggtgctgttggagtttgtgacagctgcaggcatcactttgggcatggatgaactttacaaatgattaattaactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcccttgtttccacagacaagagtgtctagagcatggctacgtagataagtagcatggcgggttaatcattaactacacctgcaggaggaacccctagtgatggagttggccactccctctctgcgcgctcgctcgctcactgaggccgggcgaccaaaggtcgcccgacgcccgggctttgcccgggcggcctcagtgag

SEQ ID NO: 44 - DNA, 2226 bp, synthetic construct
  source 1..2226: mol_type = other DNA
ctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatcgcggccgcgttacaggaaaatctgaaggaggagatctcttaggtcagtgaagagaagaacaaaaagcagcatattacagttagttgtcttcatcaatctttaaatatgttgtgtggtttttctctccctgtttccacagttgtttccaagggggaggagctgtttacaggtgtggtgcccatcctggttgaactggatggggatgtgaatggccacaagttctctgtttctggagaaggagagggagatgccacatatgggaagctcaccctgaaatttatctgcactactggcaagcttccagtgccttggcccacacttgtgactacattgacctatggagtccagtgtttcagcaggtaccctgaccacatgaagcagcatgacttttttaagagtgccatgcctgagggctatgtgcaggagagaaccatcttcttcaaggatgatggcaactacaagaccagagctgaggtgaagtttgaaggtgacacccttgtcaacaggatagaactaaagggtattgacttcaaagaagatggaaatattcttggccataagctggagtacaattacaactcccacaatgtctacatcatggctgacaagcagaagaatgggatcaaggtgaacttcaagatcaggcacaacattgaggatggatctgtgcagctggctgatcactaccagcagaacacacccattggtgatggtcctgtcctgctcccagacaaccactatttatccacccagtcagccctgtcaaaggacccaaatgagaagagggaccatatggtgctgttggagtttgtgacagctgcaggcatcactttgggcatggatgaactttacaaatgattaattaactgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttactgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgccctccttcagattttcctgtaactctagagcatggctacgtagataagtagcatggcgggttaatcattaactacacctgcaggaggaacccctagtgatggagttggccactccctctctgcgcgctcgctcgctcactgaggccgggcgaccaaaggtcgcccgacgcccgggctttgcccgggcggcctcagtgag

SEQ ID NO: 45 - DNA, 123 bp, synthetic construct
  source 1..123: mol_type = other DNA
caaactgttaatcctaacctaggttaagctacaccaaagagctgctgtcctgtggaattctgtagatcacagagacaaggcctgtgattgctgaacctgtatttgcatctgagaacccttagg

SEQ ID NO: 46 - DNA, 123 bp, synthetic construct
  source 1..123: mol_type = other DNA
cctaagggttctcagatgcaaataatatcaaagtgtccttattggatccttggctcctaaggttcacctcccagcacctggacagttgggcctgagatattgggttgtaagtggctggaacca

SEQ ID NO: 47 - DNA, 23 bp, synthetic construct
  source 1..23: mol_type = other DNA
atttgcatctgagaacccttagg

SEQ ID NO: 48 - DNA, 23 bp, synthetic construct
  source 1..23: mol_type = other DNA
cctaagggttctcagatgcaaat

SEQ ID NO: 49 - DNA, 123 bp, synthetic construct; identical to SEQ ID NO: 45
  source 1..123: mol_type = other DNA

SEQ ID NO: 50 - DNA, 123 bp, synthetic construct; identical to SEQ ID NO: 46
  source 1..123: mol_type = other DNA

SEQ ID NO: 51 - DNA, 23 bp, synthetic construct
  source 1..23: mol_type = other DNA
atttgcatctgagaacccttagg

SEQ ID NO: 52 - DNA, 23 bp, synthetic construct
  source 1..23: mol_type = other DNA
cctaagggttctcagatgcaaat

SEQ ID NO: 53 - DNA, 123 bp, synthetic construct
  source 1..123: mol_type = other DNA
caaactgttaatcctaacctaggttaagctacaccaaagagctgctgtcctgtggaattctgtagatcacagagacaaggcctgtgattgctgaacctgtcactcttgtctgtggaaacaagg

SEQ ID NO: 54 - DNA, 123 bp, synthetic construct
  source 1..123: mol_type = other DNA
ccttgtttccacagacaagagtgaatatcaaagtgtccttattggatccttggctcctaaggttcacctcccagcacctggacagttgggcctgagatattgggttgtaagtggctggaacca

SEQ ID NO: 55 - DNA, 23 bp, synthetic construct
  source 1..23: mol_type = other DNA
cactcttgtctgtggaaacaagg

SEQ ID NO: 56 - DNA, 23 bp, synthetic construct
  source 1..23: mol_type = other DNA
ccttgtttccacagacaagagtg

SEQ ID NO: 57 - DNA, 123 bp, synthetic construct
  source 1..123: mol_type = other DNA
caaactgttaatcctaacctaggttaagctacaccaaagagctgctgtcctgtggaattctgtagatcacagagacaaggcctgtgattgctgaacctgttcactcttgtctgtggaaacagg

SEQ ID NO: 58 - DNA, 123 bp, synthetic construct; identical to SEQ ID NO: 54
  source 1..123: mol_type = other DNA

SEQ ID NO: 59 - DNA, 23 bp, synthetic construct
  source 1..23: mol_type = other DNA
tcactcttgtctgtggaaacagg

SEQ ID NO: 60 - DNA, 23 bp, synthetic construct
  source 1..23: mol_type = other DNA
ccttgtttccacagacaagagtg

SEQ ID NO: 61 - DNA, 123 bp, synthetic construct
  source 1..123: mol_type = other DNA
caaactgttaatcctaacctaggttaagctacaccaaagagctgctgtcctgtggaattctgtagatcacagagacaaggcctgtgattgctgaacctgtcctaagggttctcagatgcaaat

SEQ ID NO: 62 - DNA, 123 bp, synthetic construct
  source 1..123: mol_type = other DNA
atttgcatctgagaacccttaggaatatcaaagtgtccttattggatccttggctcctaaggttcacctcccagcacctggacagttgggcctgagatattgggttgtaagtggctggaacca

SEQ ID NO: 63 - DNA, 23 bp, synthetic construct
  source 1..23: mol_type = other DNA
cctaagggttctcagatgcaaat

SEQ ID NO: 64 - DNA, 23 bp, synthetic construct
  source 1..23: mol_type = other DNA
atttgcatctgagaacccttagg

SEQ ID NO: 65 - DNA, 152 bp, synthetic construct
  source 1..152: mol_type = other DNA
taacccgccatgctacttatcgcggccgccaaactgttaatcctaacctaggttaagctacaccaaagagctgctgtcctgtggaattctgtagatcacagagacaaggcctgtgattgctgaacctgtcctaagggttctcagatgcaaat

SEQ ID NO: 66 - DNA, 123 bp, synthetic construct; identical to SEQ ID NO: 62
  source 1..123: mol_type = other DNA

SEQ ID NO: 67 - DNA, 23 bp, synthetic construct
  source 1..23: mol_type = other DNA
cctaagggttctcagatgcaaat

SEQ ID NO: 68 - DNA, 23 bp, synthetic construct
  source 1..23: mol_type = other DNA
atttgcatctgagaacccttagg

SEQ ID NO: 69 - DNA, 131 bp, synthetic construct
  source 1..131: mol_type = other DNA
caaactgttaatcctaacctaggttaagctacaccaaagagctgctgtcctgtggaattctgtagatcacagagacaaggcctgtgattgctgaacctgtcctccttcagattttcctgtaacgtttaaac

SEQ ID NO: 70 - DNA, 123 bp, synthetic construct
  source 1..123: mol_type = other DNA
gttacaggaaaatctgaaggaggaatatcaaagtgtccttattggatccttggctcctaaggttcacctcccagcacctggacagttgggcctgagatattgggttgtaagtggctggaacca

SEQ ID NO: 71 - DNA, 31 bp, synthetic construct
  source 1..31: mol_type = other DNA
cctccttcagattttcctgtaacgtttaaac

SEQ ID NO: 72 - DNA, 23 bp, synthetic construct
  source 1..23: mol_type = other DNA
gttacaggaaaatctgaaggagg

SEQ ID NO: 73 - DNA, 123 bp, synthetic construct
  source 1..123: mol_type = other DNA
caaactgttaatcctaacctaggttaagctacaccaaagagctgctgtcctgtggaattctgtagatcacagagacaaggcctgtgattgctgaacctgtgttacaggaaaatctgaaggagg

SEQ ID NO: 74 - DNA, 123 bp, synthetic construct
  source 1..123: mol_type = other DNA
cctccttcagattttcctgtaacaatatcaaagtgtccttattggatccttggctcctaaggttcacctcccagcacctggacagttgggcctgagatattgggttgtaagtggctggaacca

SEQ ID NO: 75 - DNA, 23 bp, synthetic construct
  source 1..23: mol_type = other DNA
gttacaggaaaatctgaaggagg

SEQ ID NO: 76 - DNA, 23 bp, synthetic construct
  source 1..23: mol_type = other DNA
cctccttcagattttcctgtaac

SEQ ID NO: 77 - DNA, 123 bp, synthetic construct; identical to SEQ ID NO: 45
  source 1..123: mol_type = other DNA

SEQ ID NO: 78 - DNA, 123 bp, synthetic construct; identical to SEQ ID NO: 46
  source 1..123: mol_type = other DNA

SEQ ID NO: 79 - DNA, 23 bp, synthetic construct
  source 1..23: mol_type = other DNA
atttgcatctgagaacccttagg

SEQ ID NO: 80 - DNA, 23 bp, synthetic construct
  source 1..23: mol_type = other DNA
cctaagggttctcagatgcaaat

SEQ ID NO: 81 - DNA, 123 bp, synthetic construct; identical to SEQ ID NO: 45
  source 1..123: mol_type = other DNA

SEQ ID NO: 82 - DNA, 123 bp, synthetic construct; identical to SEQ ID NO: 46
  source 1..123: mol_type = other DNA

SEQ ID NO: 83 - DNA, 23 bp, synthetic construct
  source 1..23: mol_type = other DNA
atttgcatctgagaacccttagg

SEQ ID NO: 84 - DNA, 23 bp, synthetic construct
  source 1..23: mol_type = other DNA
cctaagggttctcagatgcaaat

SEQ ID NO: 85 - DNA, 123 bp, synthetic construct; identical to SEQ ID NO: 73
  source 1..123: mol_type = other DNA

SEQ ID NO: 86 - DNA, 123 bp, synthetic construct; identical to SEQ ID NO: 74
  source 1..123: mol_type = other DNA

SEQ ID NO: 87 - DNA, 23 bp, synthetic construct
  source 1..23: mol_type = other DNA
gttacaggaaaatctgaaggagg

SEQ ID NO: 88 - DNA, 23 bp, synthetic construct
  source 1..23: mol_type = other DNA
cctccttcagattttcctgtaac

SEQ ID NO: 89 - DNA, 123 bp, synthetic construct; identical to SEQ ID NO: 53
  source 1..123: mol_type = other DNA

SEQ ID NO: 90 - DNA, 123 bp, synthetic construct; identical to SEQ ID NO: 54
  source 1..123: mol_type = other DNA

SEQ ID NO: 91 - DNA, 23 bp, synthetic construct
  source 1..23: mol_type = other DNA
cactcttgtctgtggaaacaagg

SEQ ID NO: 92 - DNA, 23 bp, synthetic construct
  source 1..23: mol_type = other DNA
ccttgtttccacagacaagagtg

SEQ ID NO: 93 - DNA, 23 bp, synthetic construct
  source 1..23: mol_type = other DNA
ccttgtttccacagacaagagtg

SEQ ID NO: 94 - DNA, 23 bp, synthetic construct
  source 1..23: mol_type = other DNA
gttacaggaaaatctgaaggagg

SEQ ID NO: 95 - DNA, 23 bp, synthetic construct
  source 1..23: mol_type = other DNA
cctccttcagattttcctgtaac

SEQ ID NO: 96 - DNA, 35 bp, synthetic construct
  source 1..35: mol_type = other DNA
tctttgtggttaggactttgagtcataagaaattt

SEQ ID NO: 97 - DNA, 144 bp, Adeno-associated virus 6
  source 1..144: mol_type = genomic DNA
atacccctagtgatggagttgcccactccctctatgcgcgctcgctcgctcggtggggccggcagaggagacctctgccgtctgcggacctttggtccgcaggccccaccgagcgagcgagcgcgcattagagggagtgggcaa

SEQ ID NO: 98 - DNA, 143 bp, Adeno-associated virus 1
  source 1..143: mol_type = genomic DNA
ttgcccactccctctctgcgcgctcgctcgctcggtggggcctgcggaccaaaggtccgcagacggcagaggtctcctctgccggccccaccgagcgagcgacgcgcgcagagagggagtgggcaactccatcactagggtaa

SEQ ID NO: 99 - DNA, 143 bp, Adeno-associated virus 1
  source 1..143: mol_type = genomic DNA
ttaccctagtgatggagttgcccactccctctctgcgcgcgtcgctcgctcggtggggccggcagaggagacctctgccgtctgcggacctttggtccgcaggccccaccgagcgagcgagcgcgcagagagggagtgggcaa

SEQ ID NO: 100 - DNA, 141 bp, Adeno-associated virus 2
  source 1..141: mol_type = genomic DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 101 - DNA, 141 bp, Adeno-associated virus 2
  source 1..141: mol_type = genomic DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 102 - DNA, 144 bp, Adeno-associated virus 3
  source 1..144: mol_type = genomic DNA
ttggccactccctctatgcgcactcgctcgctcggtggggcctggcgaccaaaggtcgccagacggacgtgggtttccacgtccggccccaccgagcgagcgagtgcgcatagagggagtggccaactccatcactagaggtat

SEQ ID NO: 103 - DNA, 144 bp, Adeno-associated virus 3
  source 1..144: mol_type = genomic DNA
atacctctagtgatggagttggccactccctctatgcgcactcgctcgctcggtggggccggacgtggaaacccacgtccgtctggcgacctttggtcgccaggccccaccgagcgagcgagtgcgcatagagggagtggccaa

SEQ ID NO: 104 - DNA, 127 bp, Adeno-associated virus 4
  source 1..127: mol_type = genomic DNA
ttggccactccctctatgcgcgctcgctcactcactcggccctggagaccaaaggtctccagactgccggcctctggccggcagggccgagtgagtgagcgagcgcgcatagagggagtggccaact

SEQ ID NO: 105 - DNA, 127 bp, Adeno-associated virus 4
  source 1..127: mol_type = genomic DNA
agttggccacattagctatgcgcgctcgctcactcactcggccctggagaccaaaggtctccagactgccggcctctggccggcagggccgagtgagtgagcgagcgcgcatagagggagtggccaa

SEQ ID NO: 106 - DNA, 166 bp, Adeno-associated virus 5
  source 1..166: mol_type = genomic DNA
tcccccctgtcgcgttcgctcgctcgctggctcgtttgggggggcgacggccagagggccgtcgtctggcagctctttgagctgccacccccccaaacgagccagcgagcgagcgaacgcgacaggggggagagtgccacactctcaagcaagggggttttgtaag

SEQ ID NO: 107 - DNA, 166 bp, Adeno-associated virus 5
  source 1..166: mol_type = genomic DNA
cttacaaaacccccttgcttgagagtgtggcactctcccccctgtcgcgttcgctcgctcgctggctcgtttgggggggtggcagctcaaagagctgccagacgacggccctctggccgtcgcccccccaaacgagccagcgagcgagcgaacgcgacagggggga

SEQ ID NO: 108 - DNA, 144 bp, Adeno-associated virus 6
  source 1..144: mol_type = genomic DNA
ttgcccactccctctaatgcgcgctcgctcgctcggtggggcctgcggaccaaaggtccgcagacggcagaggtctcctctgccggccccaccgagcgagcgagcgcgcatagagggagtgggcaactccatcactaggggtat

SEQ ID NO: 109 - DNA, 20 bp, synthetic construct
  source 1..20: mol_type = other DNA
gcatgccatcatgacttcag

SEQ ID NO: 110 - DNA, 120 bp, synthetic construct
  source 1..120: mol_type = other DNA
aggaacccctagtgatggagttggccactccctctctgcgcgctcgctcgctcactgaggcgcacgcccgggtttcccgggcggcctcagtgagcgagcgagcgcgcagctgcctgcagg

SEQ ID NO: 111 - DNA, 122 bp, synthetic construct
  source 1..122: mol_type = other DNA
aggaacccctagtgatggagttggccactccctctctgcgcgctcgctcgctcactgaggccgacgcccgggctttgcccgggcggcctcagtgagcgagcgagcgcgcagctgcctgcagg

SEQ ID NO: 112 - DNA, 129 bp, synthetic construct
  source 1..129: mol_type = other DNA
aggaacccctagtgatggagttggccactccctctctgcgcgctcgctcgctcactgaggccgggcgaccaaaggtcgcccgacgcccgggcgcctcagtgagcgagcgagcgcgcagctgcctgcagg

SEQ ID NO: 113 - DNA, 101 bp, synthetic construct
  source 1..101: mol_type = other DNA
aggaacccctagtgatggagttggccactccctctctgcgcgctcgctcgctcactgaggctttgcctcagtgagcgagcgagcgcgcagctgcctgcagg

SEQ ID NO: 114 - DNA, 139 bp, synthetic construct
  source 1..139: mol_type = other DNA
aggaacccctagtgatggagttggccactccctctctgcgcgctcgctcgctcactgaggccgggcgacaaagtcgcccgacgcccgggctttgcccgggcggcctcagtgagcgagcgagcgcgcagctgcctgcagg

SEQ ID NO: 115 - DNA, 137 bp, synthetic construct
  source 1..137: mol_type = other DNA
aggaacccctagtgatggagttggccactccctctctgcgcgctcgctcgctcactgaggccgggcgaaaatcgcccgacgcccgggctttgcccgggcggcctcagtgagcgagcgagcgcgcagctgcctgcagg

SEQ ID NO: 116 - DNA, 135 bp, synthetic construct
  source 1..135: mol_type = other DNA
aggaacccctagtgatggagttggccactccctctctgcgcgctcgctcgctcactgaggccgggcgaaacgcccgacgcccgggctttgcccgggcggcctcagtgagcgagcgagcgcgcagctgcctgcagg

SEQ ID NO: 117 - DNA, 133 bp, synthetic construct
  source 1..133: mol_type = other DNA
aggaacccctagtgatggagttggccactccctctctgcgcgctcgctcgctcactgaggccgggcaaagcccgacgcccgggctttgcccgggcggcctcagtgagcgagcgagcgcgcagctgcctgcagg

SEQ ID NO: 118 - DNA, 139 bp, synthetic construct
  source 1..139: mol_type = other DNA
aggaacccctagtgatggagttggccactccctctctgcgcgctcgctcgctcactgaggccgggcgaccaaaggtcgcccgacgcccgggtttcccgggcggcctcagtgagcgagcgagcgcgcagctgcctgcagg

SEQ ID NO: 119 - DNA, 137 bp, synthetic construct
  source 1..137: mol_type = other DNA
aggaacccctagtgatggagttggccactccctctctgcgcgctcgctcgctcactgaggccgggcgaccaaaggtcgcccgacgcccggtttccgggcggcctcagtgagcgagcgagcgcgcagctgcctgcagg

SEQ ID NO: 120 - DNA, 135 bp, synthetic construct
  source 1..135: mol_type = other DNA
aggaacccctagtgatggagttggccactccctctctgcgcgctcgctcgctcactgaggccgggcgaccaaaggtcgcccgacgcccgtttcgggcggcctcagtgagcgagcgagcgcgcagctgcctgcagg

SEQ ID NO: 121 - DNA, 133 bp, synthetic construct
  source 1..133: mol_type = other DNA
aggaacccctagtgatggagttggccactccctctctgcgcgctcgctcgctcactgaggccgggcgaccaaaggtcgcccgacgccctttgggcggcctcagtgagcgagcgagcgcgcagctgcctgcagg

SEQ ID NO: 122 - DNA, 131 bp, synthetic construct
  source 1..131: mol_type = other DNA
aggaacccctagtgatggagttggccactccctctctgcgcgctcgctcgctcactgaggccgggcgaccaaaggtcgcccgacgcctttggcggcctcagtgagcgagcgagcgcgcagctgcctgcagg

SEQ ID NO: 123 - DNA, 129 bp, synthetic construct
  source 1..129: mol_type = other DNA
aggaacccctagtgatggagttggccactccctctctgcgcgctcgctcgctcactgaggccgggcgaccaaaggtcgcccgacgctttgcggcctcagtgagcgagcgagcgcgcagctgcctgcagg

SEQ ID NO: 124 - DNA, 127 bp, synthetic construct
  source 1..127: mol_type = other DNA
aggaacccctagtgatggagttggccactccctctctgcgcgctcgctcgctcactgaggccgggcgaccaaaggtcgcccgacgtttcggcctcagtgagcgagcgagcgcgcagctgcctgcagg

SEQ ID NO: 125 - DNA, 122 bp, synthetic construct
  source 1..122: mol_type = other DNA
aggaacccctagtgatggagttggccactccctctctgcgcgctcgctcgctcactgaggccgggcgaccaaaggtcgcccgacggcctcagtgagcgagcgagcgcgcagctgcctgcagg

SEQ ID NO: 126 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggcggcctcagtgagcgagcgagcgcgcagctgcctgcagg

SEQ ID NO: 127 - DNA, 120 bp, synthetic construct
  source 1..120: mol_type = other DNA
cctgcaggcagctgcgcgctcgctcgctcactgaggccgcccgggaaacccgggcgtgcgcctcagtgagcgagcgagcgcgcagagagggagtggccaactccatcactaggggttcct

SEQ ID NO: 128 - DNA, 122 bp, synthetic construct
  source 1..122: mol_type = other DNA
cctgcaggcagctgcgcgctcgctcgctcactgaggccgtcgggcgacctttggtcgcccggcctcagtgagcgagcgagcgcgcagagagggagtggccaactccatcactaggggttcct

SEQ ID NO: 129 - DNA, 122 bp, synthetic construct
  source 1..122: mol_type = other DNA
cctgcaggcagctgcgcgctcgctcgctcactgaggccgcccgggcaaagcccgggcgtcggcctcagtgagcgagcgagcgcgcagagagggagtggccaactccatcactaggggttcct

SEQ ID NO: 130 - DNA, 129 bp, synthetic construct
  source 1..129: mol_type = other DNA
cctgcaggcagctgcgcgctcgctcgctcactgaggcgcccgggcgtcgggcgacctttggtcgcccggcctcagtgagcgagcgagcgcgcagagagggagtggccaactccatcactaggggttcct

SEQ ID NO: 131 - DNA, 101 bp, synthetic construct
  source 1..101: mol_type = other DNA
cctgcaggcagctgcgcgctcgctcgctcactgaggcaaagcctcagtgagcgagcgagcgcgcagagagggagtggccaactccatcactaggggttcct

SEQ ID NO: 132 - DNA, 139 bp, synthetic construct
  source 1..139: mol_type = other DNA
cctgcaggcagctgcgcgctcgctcgctcactgaggccgcccgggcaaagcccgggcgtcgggcgactttgtcgcccggcctcagtgagcgagcgagcgcgcagagagggagtggccaactccatcactaggggttcct

SEQ ID NO: 133 - DNA, 137 bp, synthetic construct
  source 1..137: mol_type = other DNA
cctgcaggcagctgcgcgctcgctcgctcactgaggccgcccgggcaaagcccgggcgtcgggcgattttcgcccggcctcagtgagcgagcgagcgcgcagagagggagtggccaactccatcactaggggttcct

SEQ ID NO: 134 - DNA, 135 bp, synthetic construct
  source 1..135: mol_type = other DNA
cctgcaggcagctgcgcgctcgctcgctcactgaggccgcccgggcaaagcccgggcgtcgggcgtttcgcccggcctcagtgagcgagcgagcgcgcagagagggagtggccaactccatcactaggggttcct

SEQ ID NO: 135 - DNA, 133 bp, synthetic construct
  source 1..133: mol_type = other DNA
cctgcaggcagctgcgcgctcgctcgctcactgaggccgcccgggcaaagcccgggcgtcgggctttgcccggcctcagtgagcgagcgagcgcgcagagagggagtggccaactccatcactaggggttcct

SEQ ID NO: 136 - DNA, 139 bp, synthetic construct
  source 1..139: mol_type = other DNA
cctgcaggcagctgcgcgctcgctcgctcactgaggccgcccgggaaacccgggcgtcgggcgacctttggtcgcccggcctcagtgagcgagcgagcgcgcagagagggagtggccaactccatcactaggggttcct

SEQ ID NO: 137 - DNA, 137 bp, synthetic construct
  source 1..137: mol_type = other DNA
cctgcaggcagctgcgcgctcgctcgctcactgaggccgcccggaaaccgggcgtcgggcgacctttggtcgcccggcctcagtgagcgagcgagcgcgcagagagggagtggccaactccatcactaggggttcct

SEQ ID NO: 138 - DNA, 135 bp, synthetic construct
  source 1..135: mol_type = other DNA
cctgcaggcagctgcgcgctcgctcgctcactgaggccgcccgaaacgggcgtcgggcgacctttggtcgcccggcctcagtgagcgagcgagcgcgcagagagggagtggccaactccatcactaggggttcct

SEQ ID NO: 139 - DNA, 133 bp, synthetic construct
  source 1..133: mol_type = other DNA
cctgcaggcagctgcgcgctcgctcgctcactgaggccgcccaaagggcgtcgggcgacctttggtcgcccggcctcagtgagcgagcgagcgcgcagagagggagtggccaactccatcactaggggttcct

SEQ ID NO: 140 - DNA, 131 bp, synthetic construct
  source 1..131: mol_type = other DNA
cctgcaggcagctgcgcgctcgctcgctcactgaggccgccaaaggcgtcgggcgacctttggtcgcccggcctcagtgagcgagcgagcgcgcagagagggagtggccaactccatcactaggggttcct

SEQ ID NO: 141 - DNA, 129 bp, synthetic construct
  source 1..129: mol_type = other DNA
cctgcaggcagctgcgcgctcgctcgctcactgaggccgcaaagcgtcgggcgacctttggtcgcccggcctcagtgagcgagcgagcgcgcagagagggagtggccaactccatcactaggggttcct

SEQ ID NO: 142 - DNA, 127 bp, synthetic construct
  source 1..127: mol_type = other DNA
cctgcaggcagctgcgcgctcgctcgctcactgaggccgaaacgtcgggcgacctttggtcgcccggcctcagtgagcgagcgagcgcgcagagagggagtggccaactccatcactaggggttcct

SEQ ID NO: 143 - DNA, 12 bp, synthetic construct
  source 1..12: mol_type = other DNA
atcgaacgatcg

SEQ ID NO: 144 - DNA, 12 bp, synthetic construct
  source 1..12: mol_type = other DNA
cgatcgttcgat

SEQ ID NO: 145 - DNA, 12 bp, synthetic construct
  source 1..12: mol_type = other DNA
atcgaaccatcg

SEQ ID NO: 146 - DNA, 137 bp, synthetic construct
  source 1..137: mol_type = other DNA
nnnnnnnnnnnnnnnnnnnngtttttgtactctcaagatttagaaataaatcttgcagaagctacaaagataaggcttcatgccgaaatcaacaccctgtcattttatggcagggtgttttcgttatttaatttttt

SEQ ID NO: 147 - DNA, 123 bp, synthetic construct
  source 1..123: mol_type = other DNA
nnnnnnnnnnnnnnnnnnnngtttttgtactctcagaaathcagaagctacaaagataaggcttcatgccgaaatcaacaccctgtcattttatggcagggtgttttcgttatttaatttttt

SEQ ID NO: 148 - DNA, 110 bp, synthetic construct
  source 1..110: mol_type = other DNA
nnnnnnnnnnnnnnnnnnnngtttttgtactctcagaaatgcagaagctacaaagataaggcttcatgccgaaatcaacaccctgtcattttatggcagggtgttttttt

SEQ ID NO: 149 - DNA, 102 bp, synthetic construct
  source 1..102: mol_type = other DNA
nnnnnnnnnnnnnnnnnnnngttttagagctagaaatagcaagttaaaataaggctagtccgttatcaacttgaaaaagtggcaccgagtcggtgctttttt

SEQ ID NO: 150 - DNA, 87 bp, synthetic construct
  source 1..87: mol_type = other DNA
nnnnnnnnnnnnnnnnnnnngttttagagctagaaatagcaagttaaaataaggctagtccgttatcaacttgaaaaagtttttttt

SEQ ID NO: 151 - DNA, 76 bp, synthetic construct
  source 1..76: mol_type = other DNA
nnnnnnnnnnnnnnnnnnnngttttagagctagaaatagcaagttaaaataaggctagtccgttatcatttttttt

SEQ ID NO: 152 - protein, 7 aa, Simian virus 40
PKKKRKV

SEQ ID NO: 153 - DNA, 21 bp, Simian virus 40
  source 1..21: mol_type = genomic DNA
cccaagaagaagaggaaggtg

SEQ ID NO: 154 - protein, 16 aa, unidentified
KRPAATKKAGQAKKKK

SEQ ID NO: 155 - protein, 9 aa, unidentified
PAAKRVKLD

SEQ ID NO: 156 - protein, 11 aa, unidentified
RQRRNELKRSP

SEQ ID NO: 157 - protein, 38 aa, Homo sapiens
NQSSNFGPMKGGNFGGRSSGPYGGGGQYFAKPRNQGGY

SEQ ID NO: 158 - protein, 42 aa, unidentified
RMRIZFKNKGKDTAELRRRRVEVSVELRKAKKDEQILKRRNV

SEQ ID NO: 159 - protein, 8 aa, unidentified
VSRKRPRP

SEQ ID NO: 160 - protein, 8 aa, unidentified
PPKKARED

SEQ ID NO: 161 - protein, 8 aa, Homo sapiens
PQPKKKPL

SEQ ID NO: 162 - protein, 12 aa, Mus musculus
SALIKKKKKMAP

SEQ ID NO: 163 - protein, 5 aa, Influenza A virus
DRLRR

SEQ ID NO: 164 - protein, 7 aa, Influenza A virus
PKQKKRK

SEQ ID NO: 165 - protein, 10 aa, Hepacivirus D
RKLKKKIKKL

SEQ ID NO: 166 - protein, 10 aa, Mus musculus
REKKKFLKRR

SEQ ID NO: 167 - protein, 20 aa, Homo sapiens
KRKGDEVDGVDEVAKKKSKK

SEQ ID NO: 168 - protein, 17 aa, Homo sapiens
RKCLQAGMNLEARKTKK

SEQ ID NO: 169 - DNA, 20 bp, synthetic construct
  source 1..20: mol_type = other DNA
ctccttttacgctgtgtgga

SEQ ID NO: 170 - DNA, 20 bp, synthetic construct
  source 1..20: mol_type = other DNA
tgctgctttatagcctctgt

SEQ ID NO: 171 - DNA, 20 bp, synthetic construct
  source 1..20: mol_type = other DNA
aggaggagaaaacgaaagcc

SEQ ID NO: 172 - DNA, 20 bp, synthetic construct
  source 1..20: mol_type = other DNA
cgcattgtctgagtaggtgt

SEQ ID NO: 173 - DNA, 20 bp, synthetic construct
  source 1..20: mol_type = other DNA
ggacagcaagggggaggatt

SEQ ID NO: 174 - DNA, 20 bp, synthetic construct
  source 1..20: mol_type = other DNA
atgcctgctattgtcttccc

SEQ ID NO: 175 - DNA, 20 bp, synthetic construct
  source 1..20: mol_type = other DNA
agaatccagatgctcaaggc

SEQ ID NO: 176 - DNA, 23 bp, synthetic construct
  source 1..23: mol_type = other DNA
tcccccagtttagtagttggact

SEQ ID NO: 177 - DNA, 20 bp, synthetic construct
  source 1..20: mol_type = other DNA
tcgctttcttgctgtccaat

SEQ ID NO: 178 - DNA, 20 bp, synthetic construct
  source 1..20: mol_type = other DNA
tttgatgagggacttcagcc

SEQ ID NO: 179 - DNA, 24 bp, synthetic construct
  source 1..24: mol_type = other DNA
acagtggagacattgcctactggg

SEQ ID NO: 180 - DNA, 20 bp, synthetic construct
  source 1..20: mol_type = other DNA
cattgacaaagatggctggc

SEQ ID NO: 181 - DNA, 20 bp, synthetic construct
  source 1..20: mol_type = other DNA
tgtacatgctctggaaaccc

SEQ ID NO: 182 - DNA, 31 bp, synthetic construct
  source 1..31: mol_type = other DNA
tcattatcatggacagcaagacagattacca

SEQ ID NO: 183 - DNA, 20 bp, synthetic construct
  source 1..20: mol_type = other DNA
agaagctgccaatcatccag

(19) United States
(12) Patent Application Publication
(10) Pub. No.: US 2026/0055426 A1
(43) Pub. Date: Feb. 26, 2026

(54) CLEAVABLE CLOSED-ENDED DNA (ceDNA) AND METHODS OF USE THEREOF

(71) Applicant: Generation Bio Co., Cambridge, MA (US)

(72) Inventors: Ashley Renee Penvose, Cambridge, MA (US); Russell Monds, Cambridge, MA (US)

(21) Appl. No.: 19/104,655

(22) PCT Filed: Aug. 18, 2023
(86) PCT No.: PCT/US2023/072469

§ 371 Date: Feb. 18, 2025

Related U.S. Application Data
(60) Provisional application No. 63/399,562, filed on Aug. 19, 2022.

(51) Int. Cl. C12N 15/85; A61K 35/17; A61K 48/00; A61P 1/16; C12N 9/22; C12N 15/11; C12N 15/88; C12N 15/90

(52) U.S. Cl. CPC C12N 15/85; A61K 35/17; A61P 1/16; C12N 9/226; C12N 15/11; C12N 15/88; C12N 15/907; A61K 48/00; C12N 2310/20

(57) ABSTRACT

The disclosure provides cleavable closed-ended DNA (ceDNA). In some embodiments, the cleavable ceDNA may be used as donor or repair template for editing of a target sequence in the genome.

### RELATED APPLICATIONS

This application claims the benefit of priority to U.S. Provisional Application No. 63/399,562, filed on Aug. 19, 2022. The entire contents of the foregoing application are expressly incorporated by reference herein.

### BACKGROUND

Gene editing aims to improve clinical outcomes for patients suffering from either genetic mutations or acquired diseases caused by an aberration in the gene expression profile. Gene editing includes the treatment or prevention of medical conditions resulting from defective genes or abnormal regulation or expression, e.g., underexpression or overexpression, that can result in a disorder, disease, malignancy, etc. For example, a disease or disorder caused by a defective gene might be treated, prevented or ameliorated by delivery of a corrective or designed genetic material to a patient resulting in the therapeutic effect of the genetic material within the patient. A disease or disorder caused by a defective gene might be treated, prevented or ameliorated by altering or silencing a defective gene, e.g., removing all or part of the defective gene and/or editing a specific part of the defective gene with a corrective genetic material to a patient resulting in the therapeutic expression of the genetic material within the patient.

The basis of gene editing is to supply a donor/repair template with a corrected or desired genetic sequence (sometimes referred to as a transgene), along with other editing components (e.g., guide RNA and nuclease) that facilitates editing of the genome which can result in a positive gain-of-function effect, a negative loss-of-function effect, or another outcome, such as, e.g., an oncolytic effect. Gene editing can also be used to treat a disease or malignancy caused by other factors.

Site-specific nucleases can be used to introduce site-specific double or single strand breaks at targeted genomic loci, which stimulates the natural DNA-repair machinery, leading to repair of the resultant breaks. These gene editing methods can be used to introduce specific changes in the DNA sequence at target sites. Engineered nucleases have been used for gene editing in a variety of human stem cells and cell lines, and for gene editing in the mouse liver. However, the major hurdle for implementation of these technologies is delivery to particular tissues in vivo in a way that is effective, efficient, and facilitates successful genome modification.

Current gene editing approaches, such as those utilizing AAV to deliver a repair template, are problematic and have several limitations. First, the size of the repair template and for example, the homology arms for inducing homology-directed recombination (HDR) are constrained by the packaging requirements within the AAV particle. Second, immunogenicity induced by the AAV administration precludes re-dosing and therefore, the gene editing process can only be done once. Finally, baseline immunity against AAV precludes a substantial proportion of patients from receiving the potential gene editing therapy. There have been reports of other limitations of current gene editing approaches relating to the various components such as nuclease(s), promoter(s) guide RNA(s) (if Cas9 is the nuclease), the ‘corrected gene’ repair template(s) (e.g., a homology-directed recombination (HDR) repair template) and the separate delivery of homology regions. The current delivery of components is also problematic as components cannot be packaged in a single delivery particle and the use of multiple particles can raise immunogenicity issues. Since gene editing requires all the components are present within a single cell which is to be edited, the efficiency of gene editing is low as many cells do not get all of the delivered components. Further, the current gene editing technology suffers from low rates of editing due to instability of nucleic acid encoding editing template(s) inefficiency and other components involved the editing process.

There is a great need in the field for a technology that allows precise targeting of nuclease activity (or other protein activities) to distinct locations within a target DNA in a manner that does not require the design of a new protein for each new target sequence. In addition, there is a need in the art for methods of controlling gene expression with minimal off-target effects, and there remains an important unmet need for controllable recombinant DNA vectors with improved production and/or expression properties.

### SUMMARY OF THE INVENTION

The present disclosure provides a cleavable non-viral capsid-free DNA with covalently-closed ends (referred to herein as a cleavable “closed-ended DNA” or a “ceDNA”) for use as a donor sequence for gene editing purposes. The disclosure is based, at least in part, on the inclusion of guide RNA (gRNA) target sequences (TS) and protospacer adjacent motifs (PAMs) within ceDNA to generate cleavable ceDNA constructs. The cleavable ceDNA will be delivered intact, uncleaved, alongside the sgRNA and a site-specific nuclease molecule (such as Cas9), and cleavage will occur, providing at least one open-ended transgene cassette for gene insertion.

The inventors of the instant disclosure found that when cleavable ceDNA was targeted by a nuclease, such as a Cas enzyme, and at least one gRNA, double-stranded breaks were created, resulting in cleavage of the cleavable ceDNA to produce a “cleaved ceDNA.” For example, when a Cas9 site-specific nuclease was used, this break occurred 3 base pairs upstream of the PAM. The cleaved ceDNA can then be used as a donor sequence/repair template in combination with, for example, other editing components, e.g., a nuclease and at least one gRNA, for insertion of transgene cassettes into a cell's genome, either through homology directed recombination (HDR), microhomology-mediated end joining (MMFJ), or non-homologous end joining (NHEJ) (also known as homology-independent targeted integration (HITI). The inventors of the instant disclosure surprisingly found that cleavage of cleavable ceDNA by a site-specific nuclease enzyme was more efficient than when ceDNA was used without a cleavable sequence (gRNA target sequence/PAM unit). It was found that when a spacer (stuffer) sequence with a certain length (e.g., from about 50 to about 100 bp or from 20 to about 150 bp) was placed immediately adjacent to the gRNA target sequence/PAM unit, e.g., between the gRNA target sequence/PAM and the ITRs of a cleavable ceDNA, overall editing rates were significantly increased.

The cleaved ceDNA described herein may be advantageously used as donor sequences for insertion of transgenes into the genome of a cell, either through homology directed recombination (HDR), microhomology-mediated end joining (MMEJ), or non-homologous end joining (NHEJ) (also known as homology-independent targeted integration (HITI)). Without wishing to be bound by theory, it is thought that cleaved ceDNA will integrate more efficiently than a standard ceDNA or a standard donor sequence, either by increased nuclear import resulting from Cas9-ceDNA interactions, or increased repair efficiency with an open-ended DNA insertion template (cleavable ceDNA) compared to a closed-ended insertion template (standard ceDNA, non-cleavable).

According to a first aspect, the disclosure provides a cleavable non-viral capsid-free closed-ended DNA (ceDNA) comprising a first inverted terminal repeat (ITR); a first guide RNA (gRNA) target sequence (TS) and a first protospacer adjacent motif (PAM); at least one transgene cassette; a second gRNA target sequence (TS) and a second protospacer adjacent motif (PAM); and a second ITR. According to some embodiments, the cleavable ceDNA further comprises a first spacer sequence between the first ITR and the first gRNA TS, and/or a first spacer sequence between the first PAM and the at least one transgene cassette. According to some embodiments, the first spacer sequence is between 1-200 nucleotides in length. According to some embodiments, the first spacer sequence is 10, 25, 50, 75, 100, 125, 150, 175, or 200 nucleotides in length. According to some embodiments of the aspects and embodiments herein, the first spacer sequence has at least 85% identity to a spacer sequence selected from SEQ ID NO: 45, as shown in FIG. 3B, SEQ ID NO: 49, as shown in FIG. 4B, SEQ ID NO: 53 as shown in FIG. 5B, SEQ ID NO: 57, as shown in FIG. 6B, SEQ ID NO: 61, as shown in FIG. 8B, SEQ ID NO: 65 as shown in FIG. 9B, SEQ ID NO: 69, as shown in FIG. 10B, SEQ ID NO: 73 as shown in FIG. 11B, SEQ ID NO: 77, as shown in FIG. 14B, SEQ ID NO: 81, as shown in FIG. 16B, SEQ ID NO: 85 as shown in FIG. 17B, and SEQ ID NO: 89, as shown in FIG. 18B. According to some embodiments of the aspects and embodiments herein, the cleavable ceDNA further comprises a second spacer sequence between the second gRNA TS and the second ITR, and/or a second spacer sequence between the at least one transgene cassette and the second PAM. According to some embodiments, the second spacer sequence is between 1-200 nucleotides in length. According to some embodiments, the second spacer sequence is 10, 25, 50, 75, 100, 125, 150, 175, or 200 nucleotides in length. According to some embodiments of the aspects and embodiments herein, the second spacer sequence has at least 85% identity to a spacer sequence selected from SEQ ID NO: 46, as shown in FIG. 3B, SEQ ID NO: 50, as shown in FIG. 4B, SEQ ID NO: 54 as shown in FIG. 5B, SEQ ID NO: 58, as shown in FIG. 6B, SEQ ID NO: 62, as shown in FIG. 8B, SEQ ID NO: 66 as shown in FIG. 9B, SEQ ID NO: 70, as shown in FIG. 10B, SEQ ID NO: 74 as shown in FIG. 11B, SEQ ID NO: 78, as shown in FIG. 14B, SEQ ID NO: 82, as shown in FIG. 16B, SEQ ID NO: 86 as shown in FIG. 17B, and SEQ ID NO: 90, as shown in FIG. 18B. According to some embodiments of the aspects and embodiments herein, the first spacer sequence and the second spacer sequence are the same spacer sequence or different spacer sequences. According to some embodiments of the aspects and embodiments herein, the first PAM comprises a nucleic acid sequence selected from the group consisting of: 5′-NGG-3′, 5′-NGAAA-3′, 5′-NNG-3′, 5′-NGA-3′, 5′-NTAA-3′, 5′-NTG-3′, 5′-NNC-3′ 5′-NNAAC-3′, 5′-AGA-3′, 5′-NNNANNA-3′, 5′-NNANAA-3′, 5′-NNAAAA-3′, and 5′-AAAA-3′; and/or wherein the second PAM comprises a nucleic acid sequence selected from the group consisting of: 5′-NGG-3′, 5′-NGAAA-3′, 5′-NNG-3′, 5′-NGA-3′, 5′-NTAA-3′, 5′-NTG-3′, 5′-NNC-3′, 5′-NNAAC-3′, 5′-AGA-3′, 5′-NNNANNA-3′, 5′-NNANAA-3′, 5′-NNAAAA-3′, and 5′-AAAA-3′. According to some embodiments of the aspects and embodiments herein, the first PAM and the second PAM comprise a nucleic acid sequence that is the same PAM sequence or a different PAM sequence. According to some embodiments of the aspects and embodiments herein, the first or second PAM is 3′ to the first gRNA TS. According to some embodiments of the aspects and embodiments herein, the first gRNA TS comprises a nucleic acid sequence that is the same as a nucleic acid sequence of the second gRNA TS. According to some embodiments of the aspects and embodiments herein, the first gRNA TS comprises a nucleic acid sequence that is different than a nucleic acid sequence of the second gRNA TS. According to some embodiments of the aspects and embodiments herein, the cleavable ceDNA further comprises a third gRNA target sequence (TS) and a third protospacer adjacent motif (PAM). According to some embodiments, the third gRNA target sequence (TS) and a third protospacer adjacent motif (PAM) are located within the at least one transgene cassette. According to some embodiments, the cleavable ceDNA further comprises a fourth gRNA target sequence (TS) and a fourth protospacer adjacent motif (PAM). According to some embodiments of the aspects and embodiments herein, the at least one transgene cassette comprises a 5′ homology arm, a donor sequence, and a 3′ homology arm. According to some embodiments, the 5′ homology arm and the 3′ homology arm are each between about 10 to 2000 bp in length. According to some embodiments, the 5′ homology arm and the 3′ homology arm are each between about 1000 to 2000 bp in length. According to some embodiments, the 5′homology arm and the 3′ homology arm are each between about 2 to 1000 bp in length. According to some embodiments of the aspects and embodiments herein, the 5′ homology arm comprises a splice donor site for a target locus, and wherein the 3′ homology arm comprises a splice acceptor site for a target locus. According to some embodiments of the aspects and embodiments herein, the at least one transgene cassette is capable of effecting homology directed recombination (HDR), or microhomology-mediated end joining (MMEJ) and editing of a sequence at a target locus. According to some embodiments of the aspects and embodiments herein, the at least one transgene cassette comprises a donor sequence and does not comprise a 5′ homology arm and a 3′homology arm. According to some embodiments of the aspects and embodiments herein, the donor sequence comprises a splice donor site and/or a splice acceptor site for a target locus. According to some embodiments, the at least one transgene cassette is capable of effecting homology directed recombination (HDR), or microhomology-mediated end joining (MMEJ) and editing of a sequence at a target locus. According to some embodiments of the aspects and embodiments herein, the at least one transgene cassette comprises all or a fragment of: an exon of a target gene, an intron of a target gene, a promoter region of a target gene, an enhancer region of a target gene, and/or a transcribed region of a target gene. According to some embodiments, the target gene is selected from a gene listed in Table 9. According to some embodiments of the aspects and embodiments herein, the first gRNA TS and the second gRNA TS are each a single guide RNA (sgRNA) target sequence. According to some embodiments of the aspects and embodiments herein, the third gRNA TS comprises a nucleic acid sequence that is the same as a nucleic acid sequence of the first gRNA TS and/or second gRNA TS. According to some embodiments of the aspects and embodiments herein, the third PAM comprises a sequence that is the same as the first PAM and/or the second PAM. According to some embodiments of the aspects and embodiments herein, the at least one transgene cassette further comprises a fourth gRNA TS and a fourth PAM. According to some embodiments, the fourth gRNA TS comprises a nucleic acid sequence that is the same as a nucleic acid sequence of the first gRNA TS, the second gRNA TS, and/or the third gRNA TS. According to some embodiments, the fourth PAM comprises a sequence that is the same as the first PAM, the second PAM, and/or the third PAM. According to some embodiments of the aspects and embodiments herein, the cleavable ceDNA comprises two transgene cassettes, three transgene cassettes, four transgene cassettes, or five transgene cassettes. According to some embodiments, the transgene cassettes are unidirectional or bidirectional. According to some embodiments, the cleavable ceDNA further comprises at least a third gRNA TS and a third PAM between a first transgene cassette and a second transgene cassette. According to some embodiments of the aspects and embodiments herein, the first ITR comprises a functional terminal resolution site and a Rep binding site, and/or wherein the second ITR comprises a functional terminal resolution site and a Rep binding site. According to some embodiments of the aspects and embodiments herein, the first ITR and the second ITR are symmetric or asymmetric. According to some embodiments of the aspects and embodiments herein, the first ITR and the second ITR are asymmetric, and wherein at least one of the ITRs is altered from a wild-type AAV ITR sequence by a deletion, addition, or substitution that affects the overall three-dimensional conformation of the ITR. According to some embodiments of the aspects and embodiments herein, the first ITR and/or the second ITR are derived from an AAV serotype selected from AAV1, AAV2, AAV3, AAV4, AAV5, AAV6, AAV7, AAV8, AAV9, AAV10, AAV11, and AAV12. According to some embodiments of the aspects and embodiments herein, the first ITR and/or the second ITR is synthetic. According to some embodiments of the aspects and embodiments herein, the first ITR and/or the second ITR is not a wild type ITR. According to some embodiments of the aspects and embodiments herein, the first ITR and/or the second ITR is modified by a deletion, insertion, and/or substitution in at least one of the ITR regions selected from A, A′, B, B′, C, C′, D, and D′. According to some embodiments of the aspects and embodiments herein, the first ITR and/or the second ITR is modified by a deletion of all or part of a stem-loop structure normally formed by the A, A′, B, B′ C, C′, D, and D′ regions. According to some embodiments of the aspects and embodiments herein, the first ITR and the second ITR are symmetric. According to some embodiments of the aspects and embodiments herein, the first ITR and/or the second ITR are wild type.

According to some aspects, the disclosure provides an polynucleotide comprising the cleavable ceDNA of any one of the aspects and embodiments herein.

According to some other aspects, the disclosure provides a gene editing system comprising the cleavable ceDNA of any one of the aspects and embodiments herein, or the isolated polynucleotide herein, at least one guide RNA (gRNA); and at least one site-specific nuclease enzyme or a messenger ribonucleic acid (mRNA) encoding the at least one site-specific nuclease enzyme. According to some embodiments, the at least one gRNA is capable of binding to both the first gRNA TS and the second gRNA TS. According to some embodiments, the at least one gRNA is a single guide RNA (sgRNA). According to some embodiments, of the aspects and embodiments, herein the at least one gRNA is further capable of binding to a first genomic gRNA TS in a genome of a host cell, and/or wherein the at least one gRNA is further capable of binding to both a first genomic gRNA TS in a genome of a host cell and a second genomic gRNA TS in the genome of the host cell. According to some embodiments of the aspects and embodiments herein, the gene editing system further comprises at least a second gRNA. According to some embodiments, the at least one gRNA is capable of binding to the first gRNA TS, and wherein the second gRNA is capable of binding to the second gRNA TS. According to some embodiments, the at least one gRNA is further capable of binding to a first genomic gRNA TS in a genome of a host cell, and wherein the second gRNA is further capable of binding to a second genomic gRNA TS in the genome of the host cell. According to some embodiments of the aspects and embodiments herein, the gene editing system further comprises a second gRNA and at least a third gRNA. According to some embodiments, the at least one gRNA is capable of binding to the first gRNA TS, the second gRNA is capable of binding to the second gRNA TS, and the third gRNA is capable of binding to a first genomic gRNA TS in a genome of a host cell; or wherein the at least one gRNA is capable of binding to the first gRNA TS and the second gRNA TS, and the second gRNA is capable of binding to a first genomic gRNA TS in a genome of a host cell, and the third gRNA is capable of binding to a second genomic gRNA TS in the genome of the host cell. According to some embodiments of the aspects and embodiments herein, the gene editing system further comprises a second gRNA, a third gRNA, and at least a fourth gRNA. According to some embodiments, the at least one gRNA is capable of binding to the first gRNA TS, the second gRNA is capable of binding to the second gRNA TS, the third gRNA is capable of binding to a first genomic gRNA TS in a genome of a host cell, and the at least fourth gRNA is capable of binding to a second genomic gRNA TS in the genome of the host cell. According to some embodiments of the aspects and embodiments herein, the site-specific nuclease is a Cas9 enzyme. According to some embodiments, the Cas9 enzyme is a wild-type Cas9 protein, a nicking Cas9 protein (nCas9) or a dead Cas9 protein (dCas9). According to some embodiments, the nCas9 contains a mutation in the HNH or RuVc domain of Cas. According to some embodiments, the Cas9 enzyme is a S. pyogenes Cas9. According to some embodiments of the aspects and embodiments herein, the cleavable ceDNA is formulated as a lipid nanoparticle composition. According to sonic embodiments of the aspects and embodiments herein, the at least one gRNA and the at least one site-specific nuclease enzyme or a messenger ribonucleic acid (mRNA) encoding the at least one site-specific nuclease enzyme are formulated as a lipid nanoparticle composition. According to some embodiments, the cleavable ceDNA is formulated as a first lipid nanoparticle composition and the at least one gRNA, and the at least one site-specific nuclease enzyme or a messenger ribonucleic acid (mRNA) encoding the at least one site-specific nuclease enzyme are formulated as a second lipid nanoparticle composition.

According to some aspects, the disclosure provides a lipid nanoparticle composition comprising the gene editing system of any of the aspects and embodiments herein.

According to some other aspects, the disclosure provides a lipid nanoparticle composition comprising the cleavable ceDNA of any of the aspects and embodiments herein or the isolated polynucleotide herein.

According to other aspects, the disclosure provides a cell comprising the cleavable ceDNA of any of the aspects and embodiments herein, the isolated polynucleotide herein, the gene editing system of any of the aspects and embodiments herein, or the lipid nanoparticle of any of the aspects and embodiments herein.

According to some aspects, the disclosure provides a method of editing a target nucleic acid sequence in a genome of a cell, the method comprising contacting the cell with the gene editing system of any of the aspects and embodiments herein, or the lipid nanoparticle of any of the aspects and embodiments herein, thereby editing the target nucleic acid in the genome of the cell. According to some embodiments, the at least one gRNA and the site-specific nuclease enzyme co-localize to at least the first gRNA TS of the cleavable ceDNA and the second gRNA TS of the cleavable ceDNA, and the site-specific nuclease enzyme cleaves the cleavable ceDNA in a site-specific manner, and the at least one gRNA and the site-specific nuclease enzyme co-localize to at least a first genomic gRNA TS and/or a second genomic gRNA TS at or near the target nucleic acid in the genome of the cell, and the site-specific nuclease enzyme cleaves the genome of the cell in a site-specific manner. According to some embodiments, at least one gRNA and a second gRNA are present, wherein the at least one gRNA and the site-specific nuclease enzyme co-localize to the first gRNA TS of the cleavable ceDNA, the second gRNA and the site-specific nuclease enzyme co-localize to the second gRNA TS of the cleavable ceDNA, and the site-specific nuclease enzyme cleaves the cleavable ceDNA in a site-specific manner; and the at least one gRNA and the site-specific nuclease enzyme co-localize to a first genomic gRNA TS at or near the target nucleic acid in the genome of the cell, the second gRNA and the site-specific nuclease enzyme co-localize to a second genomic gRNA TS at or near the target nucleic acid in the genome of the cell, and the site-specific nuclease enzyme cleaves the genome of the cell in a site-specific manner. According to some embodiments, at least one gRNA, a second gRNA, and a third gRNA are present, wherein the at least one gRNA and the site-specific nuclease enzyme co-localize to the first gRNA TS of the cleavable ceDNA, the second gRNA and the site-specific nuclease enzyme co-localize to the second gRNA TS of the cleavable ceDNA, and the site-specific nuclease enzyme cleaves the cleavable ceDNA in a site-specific manner, and the third gRNA and the site-specific nuclease enzyme co-localize to at least a first genomic gRNA TS and/or a second genomic gRNA TS at or near the target nucleic acid in the genome of the cell, and the site-specific nuclease enzyme cleaves the genome of the cell in a site-specific manner; or the at least one gRNA and the site-specific nuclease enzyme co-localize to the first gRNA TS of the cleavable ceDNA, the at least one gRNA and the site-specific nuclease enzyme co-localize to the second gRNA TS of the cleavable ceDNA, and the site-specific nuclease enzyme cleaves the cleavable ceDNA in a site-specific manner; and the second gRNA and the site-specific nuclease enzyme co-localize to a first genomic gRNA TS at or near the target nucleic acid in the genome of the cell, the third gRNA and the site-specific nuclease enzyme co-localize to a second genomic gRNA TS at or near the target nucleic acid in the genome of the cell, and the site-specific nuclease enzyme cleaves the genome of the cell in a site-specific manner. According to some embodiments, at least one gRNA, a second gRNA, a third gRNA, and a fourth gRNA are present, wherein the at least one gRNA and the site-specific nuclease enzyme co-localize to the first gRNA TS of the cleavable ceDNA, the second gRNA and the site-specific nuclease enzyme co-localize to the second gRNA TS of the cleavable ceDNA, and the site-specific nuclease enzyme cleaves the cleavable ceDNA in a site-specific manner, and the third gRNA and the site-specific nuclease enzyme co-localize to a first genomic gRNA TS at or near the target nucleic acid in the genome of the cell, the fourth gRNA and the site-specific nuclease enzyme co-localize to a second genomic gRNA TS at or near the target nucleic acid in the genome of the cell, and the site-specific nuclease enzyme cleaves the genome of the cell in a site-specific manner. According to some embodiments of the aspects and embodiments herein, the contacting effects non-homologous end joining (NHEJ), microhomology-mediated end-joining (MMEJ), or homology directed recombination (HDR) and editing of the target nucleic acid. According to some embodiments of the aspects and embodiments herein, the cell is a eukaryotic cell. According to some embodiments of the aspects and embodiments herein, the contacting is in vitro, ex vivo, or in vivo. According to some embodiments of the aspects and embodiments herein, the method is performed in vivo to correct a single nucleotide polymorphism (SNP) associated with a disease. According to some embodiments of the aspects and embodiments herein, the method further comprises administering the cells produced to a subject in need thereof. According to some embodiments, the subject in need thereof has a genetic disease, a viral infection, a bacterial infection, a parasitic infection, a fungal infection, a cancer, or an autoimmune disease.

According to another aspect, the disclosure provides a cell edited by the method of any one of the methods provided in the aspects and embodiments herein.

According to some aspects, the disclosure provides a method of producing a cleaved ceDNA, the method comprising contacting the cleavable ceDNA of any of the aspects and embodiments herein, or the isolated polynucleotide of any of the aspects or embodiments herein, with a site-specific nuclease enzyme and at least one guide RNA (gRNA), wherein the at least one gRNA and the site-specific nuclease enzyme co-localize to the first gRNA TS and/or the second gRNA TS of the cleavable ceDNA, and the site-specific nuclease enzyme cleaves the cleavable ceDNA in a site-specific manner, thereby producing the cleaved ceDNA. According to some embodiments, the method further comprises purifying the cleaved ceDNA. According to some embodiments, the disclosure provides a cleaved ceDNA produced by the method of an of the aspects of embodiments herein.

According to other aspects, the disclosure provides a cleavable non-viral capsid-free closed-ended DNA (ceDNA) comprising in the following order a first inverted terminal repeat (ITR); a first guide RNA (gRNA) target sequence (TS) and a first protospacer adjacent motif (PAM); at least one transgene cassette; a second gRNA target sequence (TS) and a second protospacer adjacent motif (PAM); and a second ITR.

These and other aspects of the invention are described in further detail below.

### BRIEF DESCRIPTION OF DRAWINGS

The patent or application file contains at least one drawing executed in color. Copies of this patent or patent application publication with color drawing(s) will be provided by the Office upon request and payment of the necessary fee.

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIGS. 1A-1D are schematics showing a standard ceDNA (top of FIG. 1A and FIG. 1C) compared to exemplary cleavable ceDNA constructs (FIGS. 1A-1D), which may include, e.g., gRNA target sequences (TS), protospacer adjacent motifs (PAMs), spacers, and transgene cassettes (with or without 5′ and 3′ homology arms or microhomology arms), in different positions and orders. For example, spacers between an ITR and a gRNA TS/PAM and/or template and a gRNA TS/PAM may help to reduce steric hindrance from the ITR or other functional cis-acting elements located near the gRNA TS/PAM for site-specific nuclease activity. FIGS. 1B and 1D show exemplary bidirectional cleavable ceDNA constructs which may include, e.g., gRNA target sequences (TS), protospacer adjacent motifs (PAMs), spacers., and bidirectional transgene cassettes (with or without 5′ and 3′ homology arms or microhomology arms), in different positions and orders.

FIGS. 2A and 2B are graphs that show the percent ceDNA cleaved by Cas9. FIG. 2A shows representative electropherograms from a bioanalyzer that showed the gain of a small fragment in the 100 bp cleavable ceDNA construct (bottom) as compared to standard ceDNA (top). FIG. 2B is a graph that shows quantification of percent ceDNA cleaved in an in vitro biochemical reaction containing ceDNA, sgRNA, and Cas9 protein. These results demonstrated that cleavable ceDNA was effectively cleaved by Cas9.

FIG. 3A shows the nucleic acid sequence of Cleavable ceDNA 344 (SEQ ID NO: 27). As shown in FIG. 3B, the first (SEQ ID NO: 45) and second (SEQ ID NO: 46) spacer sequences are bolded, and the first and second sgRNA and PAM sequences (SEQ ID NO: 47 and SEQ ID NO: 48, respectively) are bolded and underlined. FIG. 3B discloses SEQ ID NO: 27.

FIG. 4A shows the nucleic acid sequence of Cleavable ceDNA 136 (SEQ ID NO: 28). As shown in FIG. 4B, the first (SEQ ID NO: 49) and second (SEQ ID NO: 50) spacer sequences are bolded, and the first and second sgRNA and PAM sequences (SEQ ID NO: 51 and SEQ ID NO: 52, respectively) are bolded and underlined. FIG. 4B discloses SEQ ID NO: 27.

FIG. 5A shows the nucleic acid sequence of Cleavable ceDNA 137 (SEQ ID NO: 29). As shown in FIG. 5B, the first (SEQ ID NO: 53) and second (SEQ ID NO: 54) spacer sequences are bolded, and the first and second sgRNA and PAM sequences (SEQ ID NO: 55 and SEQ ID NO: 56, respectively) are bolded and underlined. FIG. 5B discloses SEQ ID NO: 29.

FIG. 6A shows the nucleic acid sequence of Cleavable ceDNA 138 (SEQ ID NO: 30). As shown in FIG. 6B, the first (SEQ ID NO: 57) and second (SEQ ID NO: 58) spacer sequences are bolded, and the first and second sgRNA and PAM sequences (SEQ ID NO: 59 and SEQ ID NO: 60, respectively) are bolded and underlined. FIG. 6B discloses SEQ ID NO: 30.

FIG. 7 shows the nucleic acid sequence of non leavable ceDNA 345 (SEQ ID NO: 31) encoding green fluorescent protein (GFP) used as control.

FIG. 8A shows the nucleic acid sequence of Cleavable ceDNA 350 (SEQ ID NO: 32). As shown in FIG. 8B, the first (SEQ ID NO: 61) and second (SEQ ID NO: 62) spacer sequences are bolded, and the first and second sgRNA and PAM sequences (SEQ ID NO: 63 and SEQ ID NO: 64, respectively) are bolded and underlined. FIG. 8A discloses SEQ ID NOS 32 and 169-183, respectively, in order of appearance. FIG. 8B discloses SEQ ID NO: 32.

FIG. 9A shows the nucleic acid sequence of Cleavable ceDNA 351 (SEQ ID NO: 33). As shown in FIG. 9B, the first (SEQ ID NO: 65) and second (SEQ ID NO: 66) spacer sequences are bolded, and the first and second sgRNA and PAM sequences (SEQ ID NO: 67 and SEQ ID NO: 68, respectively) are bolded and underlined. FIG. 9B discloses SEQ ID NO: 32.

FIG. 10A shows the nucleic acid sequence of Cleavable ceDNA 352 (SEQ ID NO: 34). As shown in FIG. 10B, the first (SEQ ID NO: 69) and second (SEQ ID NO: 70) spacer sequences are bolded, and the first and second sgRNA and PAM sequences (SEQ ID NO: 71 and SEQ ID NO: 72, respectively) are bolded and underlined. FIG. 10B discloses SEQ ID NO: 34.

FIG. 11A shows the nucleic acid sequence of Cleavable ceDNA 353(SEQ ID NO: 35). As shown in FIG. 11B, the first (SEQ ID NO: 73) and second (SEQ ID NO: 74) spacer sequences are bolded, and the first and second sgRNA and PAM sequences (SEQ ID NO: 75 and SEQ ID NO: 76, respectively) are bolded and underlined. FIG. 11B discloses SEQ ID NO: 35.

FIG. 12 shows the nucleic acid sequence of non-cleavable ceDNA 354 (SEQ ID NO: 36).

FIG. 13 shows the nucleic acid sequence of non-cleavable ceDNA 355 (SEQ ID NO: 37).

FIG. 14A shows the nucleic acid sequence of Cleavable ceDNA 356 (SEQ ID NO: 38). As shown in FIG. 14B, the first (SEQ ID NO: 77) and second (SEQ ID NO: 78) spacer sequences are bolded, and the first and second sgRNA and PAM sequences (SEQ ID NO: 79 and SEQ ID NO: 80, respectively) are bolded and underlined. FIG. 14A discloses SEQ ID NOS 38 and 172-174, respectively, in order of appearance. FIG. 14B discloses SEQ ID NO: 38.

FIG. 15 shows the nucleic acid sequence of non-cleavable ceDNA 346 (SEQ ID NO: 39).

FIG. 15 discloses SEQ ID NOS 39, 96, 109, and 175-177, respectively, in order of appearance.

FIG. 16A shows the nucleic acid sequence of Cleavable ceDNA 347 (SEQ ID NO: 40). As shown in FIG. 16B, the first (SEQ ID NO: 81) and second (SEQ ID NO: 82) spacer sequences are bolded, and the first and second sgRNA and PAM sequences (SEQ ID NO: 83 and SEQ ID NO: 84, respectively) are bolded and underlined. FIG. 16A discloses SEQ ID NOS 40, 96, 109, and 175-177, respectively, in order of appearance. FIG. 16B discloses SEQ ID NO: 40.

FIG. 17A shows the nucleic acid sequence of Cleavable ceDNA 348 (SEQ ID NO: 41). As shown in FIG. 17B, the first (SEQ ID NO: 85) and second (SEQ ID NO: 86) spacer sequences are bolded, and the first and second sgRNA and PAM sequences (SEQ ID NO: 87 and SEQ ID NO: 88, respectively) are bolded and underlined. FIG. 17B discloses SEQ ID NO: 41.

FIG. 18A shows the nucleic acid sequence of Cleavable ceDNA 349 (SEQ ID NO: 42). As shown in FIG. 18B, the first (SEQ ID NO: 89) and second (SEQ ID NO: 90) spacer sequences are bolded, and the first and second sgRNA and PAM sequences (SEQ ID NO: 91 and SEQ ID NO: 92, respectively) are bolded and underlined. FIG. 18A discloses SEQ ID NOS 42, 96, 109, and 175-177, respectively, in order of appearance. FIG. 18B discloses SEQ ID NO: 42.

FIG. 19A shows the nucleic acid sequence of Cleavable ceDNA 449 (SEQ ID NO: 43). As shown in FIG. 19B, the sgRNA and PAM sequence is bolded and underlined (SEQ ID NO: 93). FIG. 19B discloses SEQ ID NO: 43.

FIG. 20A shows the nucleic acid sequence of Cleavable ceDNA 448 (SEQ ID NO: 44). As shown in FIG. 20B, the first and second sgRNA and PAM sequences are bolded and underlined (SEQ ID NO: 94 and SEQ ID NO: 95, respectively). FIG. 20B discloses SEQ ID NO: 44.

### DETAILED DESCRIPTION

### I. Definition

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), Fields Virology, 6^{th} Edition, published by Lippincott Williams & Wilkins, Philadelphia, PA, USA (2013), Knipe, D. M. and Howley, P. M. (ed.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers. Inc., 1995 (ISBN 1-56081-569-8); Immunology by Werner Luttmann, published by Elsevier. 2006; Janeway's Immunobiology, Kenneth Murphy, Allan Mowat, Casey Weaver (eds.), Taylor & Francis Limited, 2014 (ISBN 0815345305, 9780815345305); Irwin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press. Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the terms “closed-ended DNA vector”, “ceDNA vector” and “ceDNA” are used interchangeably and refer to a non-virus capsid-free DNA vector with at least one covalently-closed end (i.e., an intramolecular duplex). In some embodiments, the ceDNA comprises two covalently-closed ends.

As used herein, the terms “transgene” and “heterologous nucleotide sequence” are used interchangeably and refer to a nucleic acid of interest that is incorporated into and may be delivered and/or expressed by a cleavable ceDNA vector as disclosed herein. A transgene sequence may comprise all, or a fragment of: an exon of a target gene, an intron of a target gene, a promoter region of a target gene, an enhancer region of a target gene, and/or a transcribed region of a target gene.

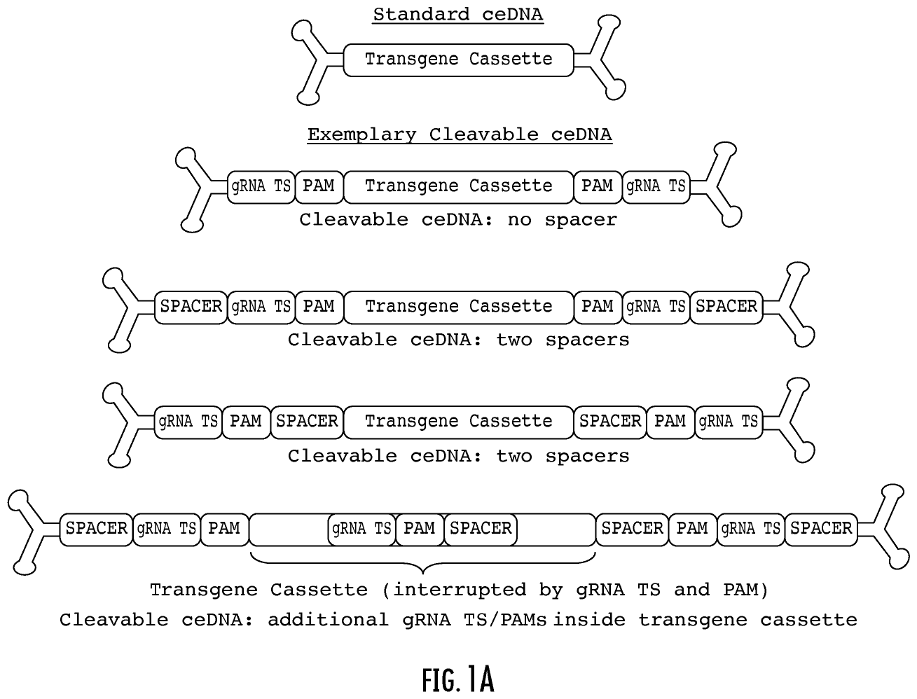

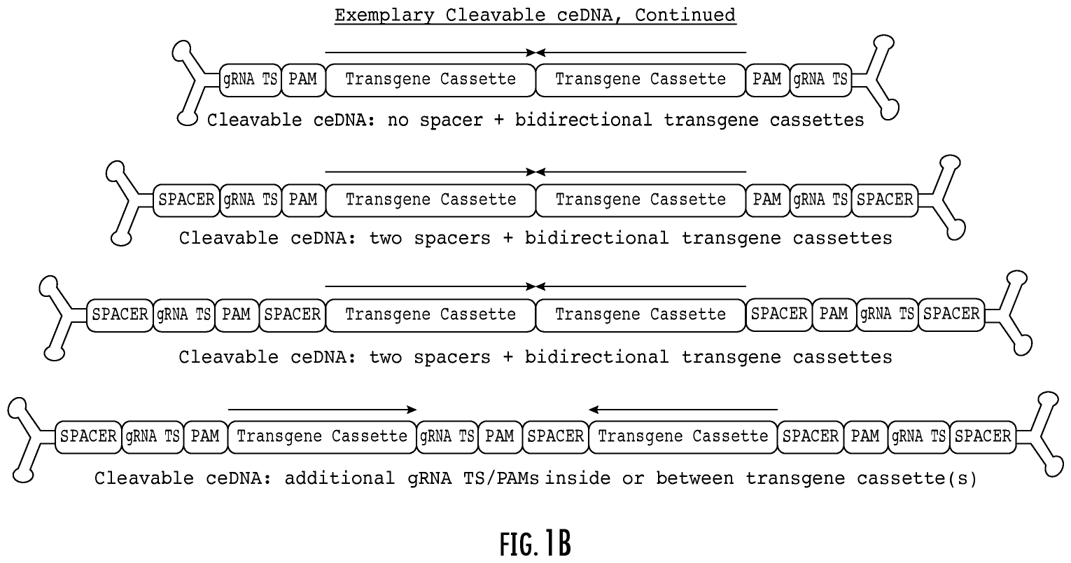

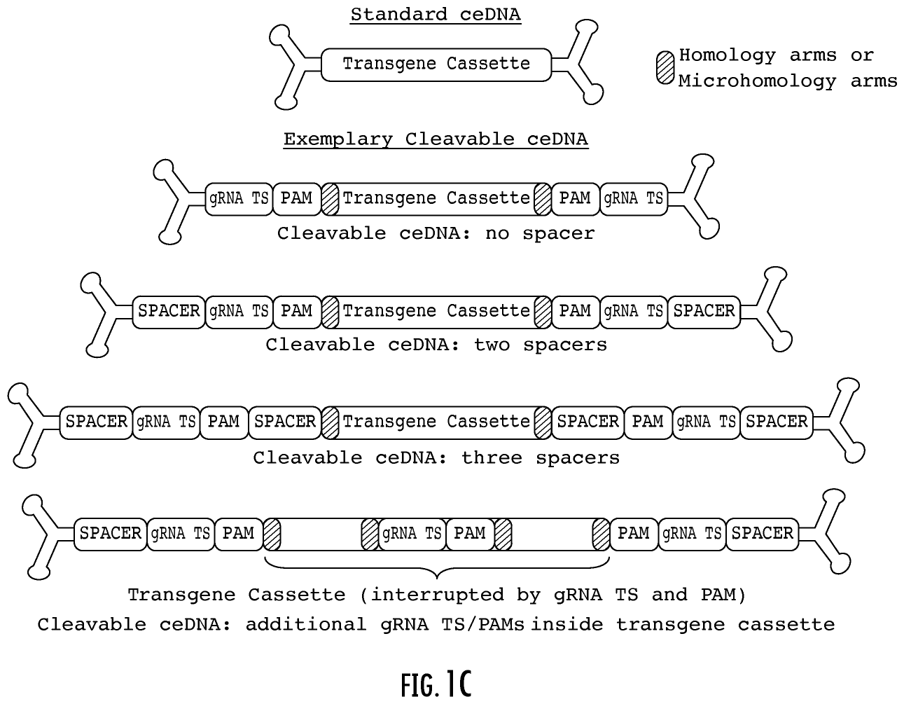

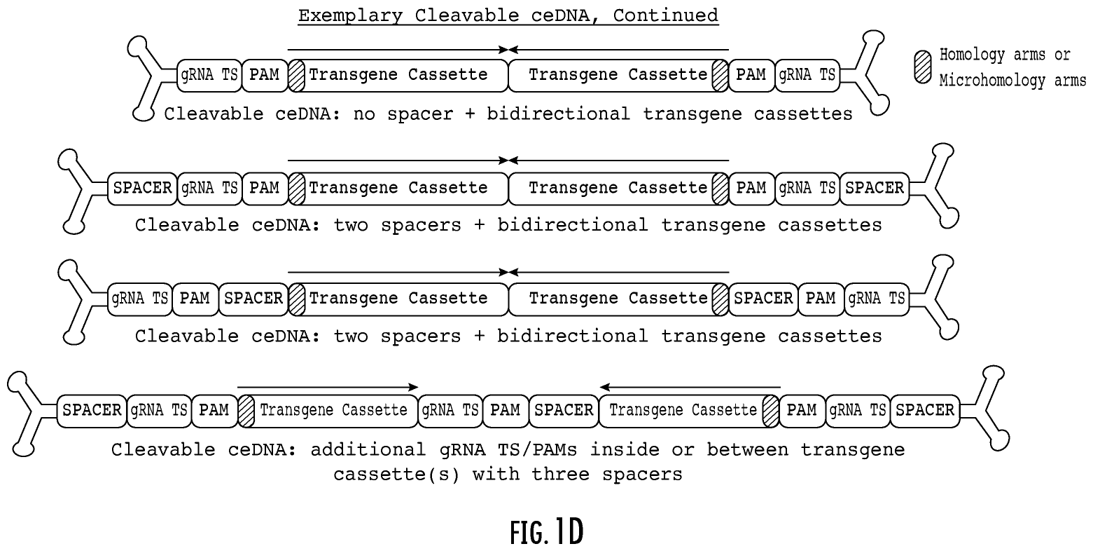

As used herein, the terms “expression cassette” and “transgene cassette” are used interchangeably and refer to a linear stretch of nucleic acids that includes a transgene that is operably linked to one or more promoters or other regulatory sequences sufficient to direct transcription of the transgene, but which does not comprise capsid-encoding sequences, other vector sequences or inverted terminal repeat regions. A transgene cassette may additionally comprise one or more cis-acting sequences (e.g., promoters, enhancers, or repressors), one or more introns, and one or more post-transcriptional regulatory elements. In one embodiment, a cleavable ceDNA vector as described herein comprises a transgene cassette that comprises two homology arms (e.g., a 5′ homology arm and a 3′ homology arm) flanking on either side of a donor sequence comprising a desired mutation or insertion in the nucleic acid sequence to be introduced into the host genome (see FIGS. 1C and 1D). In one embodiment, a cleavable ceDNA vector as described herein comprises a transgene cassette that comprises the donor sequence, but does not comprise the 5′ and 3′ homology arms (see FIGS. 1A and 1B).

The terms “polynucleotide” and “nucleic acid,” used interchangeably herein, refer to a polymeric form of nucleotides of any length, either ribonucleotides or deoxyribonucleotides. Thus, this term includes single, double, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present disclosure. An “expression cassette” includes a DNA coding sequence operably linked to a promoter.

By “hybridizable” or “complementary” or “substantially complementary” it is meant that a nucleic acid (e.g., RNA) includes a sequence of nucleotides that enables it to non-covalently bind, i.e. form Watson-Crick base pairs and/or G/J base pairs, “anneal”, or “hybridize.” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C). In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a subject DNA-targeting RNA molecule is considered complementary to a uracil (U), and vice versa. As such, when a G/U base-pair can be made at a given nucleotide position a protein-binding segment (dsRNA duplex) of a subject DNA-targeting RNA molecule, the position is not considered to be non-complementary, but is instead considered to be complementary.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

A DNA sequence that “encodes” a particular RNA or protein gene product is a DNA nucleic acid sequence that is transcribed into the particular RNA and/or protein. A DNA polynucleotide may encode an RNA (mRNA) that is translated into protein, or a DNA polynucleotide may encode an RNA that is not translated into protein (e.g., tRNA, rRNA, or a DNA-targeting RNA; also called “non-coding” RNA or “ncRNA”).

As used herein, the term “gene editing molecule” refers to one or more of a protein and/or a nucleic acid encoding for a protein, wherein the gene editing molecule is selected from the group comprising a transposase, a nuclease, an integrase, a guide RNA (gRNA), a guide DNA, donor/repair template (DNA or RNA) a ribonucleoprotein (RNP), or an activator RNA. A nuclease gene editing molecule is a protein having nuclease activity, with nonlimiting examples including: a CRISPR protein (Cas), CRISPR associated protein 9 (Cas9); a type IIS restriction enzyme; a transcription activator-like effector nuclease (TALEN); and a zinc finger nuclease (ZFN), a meganuclease, engineered site-specific nucleases or deactivated CAS for CRISPRi or CRISPRa systems. The gene editing molecule can comprise a DNA-binding domain and a nuclease. In certain embodiments, the gene editing molecule comprises a DNA donor/repair template with a sequence intended to serve as a template for correction, modification or repair of a target sequence. In certain embodiments, the DNA-binding domain comprises a guide RNA. In certain embodiments, the DNA-binding domain comprises a DNA-binding domain of a TALEN. In certain embodiments at least one gene editing molecule comprises one or more transposable element(s). In certain embodiments, the one or more transposable element(s) comprise a circular DNA. In certain embodiments, the one or more transposable element(s) comprise a plasmid vector or a minicircle DNA vector. In certain embodiments, the DNA-binding domain comprises a DNA-binding domain of a zinc-finger nuclease. In certain embodiments at least one gene editing molecule comprises one or more transposable element(s). In certain embodiments, the one or more transposable element(s) comprise a linear DNA.

As used herein, the term “gene editing functionality” refers to the insertion, deletion or replacement of DNA at a specific site in the genome with a loss or gain of function. The insertion, deletion or replacement of DNA at a specific site can be accomplished e.g., by homology-directed recombination (HDR), non-homologous end joining (NHEJ), microhomology-mediated end-joining (MMEJ), or single base change editing. In some embodiments, a repair template is used, for example for HDR, such that a desired donor sequence within the repair template is inserted into the genome by a homologous recombination event. In one embodiment, a “donor template” or “repair template” is the transgene cassette in a cleavable ceDNA vector as described herein, which comprises two homology arms (e.g., a 5′ homology arm and a 3′ homology arm) flanking on either side of a donor sequence comprising a desired mutation or insertion in the nucleic acid sequence to be introduced into the host genome (see FIGS. 1C and 1D). The 5′ and 3′ homology arms are substantially homologous to the genomic sequence of the target gene at the site of endonuclease mediated cutting. The 3′ homology arm is generally immediately downstream of the protospacer adjacent motif (PAM) site where the endonuclease cuts (e.g., a double stranded DNA cut), or in some embodiments, nicks the DNA.

“Homology-directed recombination,” “homology-directed repair,” or “HDR” as used interchangeably herein refers to a mechanism in cells to repair double strand DNA lesions when a homologous piece of DNA (e.g., homology arms of about 100 bp to 1000 bp in length) is present in the nucleus, mostly in G2 and S phase of the cell cycle. HDR uses a repair template comprising a donor sequence with flanking homology arms to guide repair and may be used to create specific sequence changes to the genome, including the targeted addition of whole genes. If a repair template is provided along with the CRISPR/Cas9-based gene editing system, then the cellular machinery will repair the break by homologous recombination, which is enhanced several orders of magnitude in the presence of DNA cleavage. When the homologous DNA piece is absent, non-homologous end joining may take place instead.

“Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible. NHEJ usually repairs the break accurately, yet imprecise repair leading to loss of nucleotides may also occur, but is much more common when the overhangs are not compatible. NHEJ can also be used to integrate transgene cassettes. For example, NHEJ can utilize a donor sequence that does not have homology arms with a target sequence, but instead includes endonuclease cleavage sites (or gRNATSs) flanking a donor sequence. Endonucleases are able to cut both the genome target sequence and the donor plasmid, after which the cleaved ceDNA can be incorporated into the target site.

“Microhomology-mediated end joining (MMEJ)” as used herein refers to a double-stranded break repair mechanism using regions with 1-100, such as 5-25 base pairs of microhomology arms flanking a double stranded break to repair DNA. The DNA ends are subject to end resection to reveal homology, allowing the strands to anneal, and DNA synthesis then fills in the gaps. MMEJ results in a deletion of the region between the microhomology and the retention of a single microhomology sequence. MMEJ can also be used to integrate a transgene cassette. For example, an endonuclease can be used to cleave both the target and cleavable donor ceDNA at sites with microhomology, resulting in precise integration into a target site.

As used herein, the term “gene editing system” refers to the minimum components necessary to effect genome editing in a cell. For example, a zinc finger nuclease or TALEN system may only require expression of the endonuclease fused to a nucleic acid complementary to the sequence of a target gene, whereas for a CRISPR/Cas gene editing system the minimum components may require e.g., a Cas endonuclease, a guide RNA, and a donor sequence (such as a cleavable ceDNA or cleaved ceDNA described herein). Those of skill in the art will readily understand the component(s) necessary for a gene editing system. In one embodiment, a gene editing system comprises a cleavable non-viral capsid-free ceDNA comprising a first inverted terminal repeat (ITR); a first guide RNA (gRNA) target sequence (TS) and a first protospacer adjacent motif (PAM); at least one transgene cassette; a second gRNA target sequence (TS) and a second protospacer adjacent motif (PAM); and a second ITR. According to some embodiments, the cleavable ceDNA further comprises a first spacer (stuffer) sequence between the first ITR and the first gRNA TS, and/or a first spacer (stuffer) sequence between the first PAM and the at least one transgene cassette. According to some embodiments, the cleavable ceDNA further comprises a second spacer (stuffer) sequence between the second gRNA TS and the second ITR, and/or a second spacer (stuffer) sequence between the at least one transgene cassette and the second PAM. According to some embodiments, the first spacer (stuffer) sequence and the second spacer (stuffer) sequence are the same spacer sequence or different spacer sequences. According to some embodiments, the first PAM and the second PAM comprise a nucleic acid sequence that is the same PAM sequence or a different PAM sequence. According to further embodiments, the first or second PAM is 3′ to the first gRNA TS. According to some embodiments, the cleavable ceDNA further comprises a third gRNA target sequence (TS) and a third protospacer adjacent motif (PAM). According to some embodiments, the third gRNA TS and a third protospacer PAM are located within the at least one transgene cassette. According to some embodiments, the cleavable ceDNA further comprises a fourth gRNA target sequence (TS) and a fourth protospacer adjacent motif (PAM).

“Clustered Regularly Interspaced Short Palindromic Repeats” and “CRISPRs”, as used interchangeably herein refers to loci containing multiple short direct repeats that are found in the genomes of approximately 40% of sequenced bacteria and 90% of sequenced archaea. The CRISPR system is a microbial nuclease system involved in defense against invading phages and plasmids that provides a form of acquired immunity. The CRISPR loci in microbial hosts contain a combination of CRISPR-associated (Cas) genes as well as non-coding RNA elements capable of programming the specificity of the CRISPR-mediated nucleic acid cleavage. Short segments of foreign DNA, called spacers, are incorporated into the genome between CRISPR repeats, and serve as a ‘memory’ of past exposures. Cas9 forms a complex with the 3 end of the sgRNA (also referred interchangeably herein as “gRNA”), and the protein-RNA pair recognizes its genomic target by complementary base pairing between the 5′ end of the sgRNA sequence and a predefined 20 bp DNA sequence, known as the protospacer. This complex is directed to homologous loci of pathogen DNA via regions encoded within the crRNA, i.e., the protospacers, and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer). By simply exchanging the 20 bp recognition sequence of the expressed sgRNA, the Cas9 nuclease can be directed to new genomic targets. CRISPR spacers are used to recognize and silence exogenous genetic elements in a manner analogous to RNAi in eukaryotic organisms.

As used herein, the term “base editing moiety” refers to an enzyme or enzyme system that can alter a single nucleotide in a sequence, for example, a cytosine/guanine nucleotide pair “G/C” to an adenine and thymine “T”/uridine “U” nucleotide pair (A/T,U) (see e.g., Shevidi et al. Dev Dyn 31 (2017) PMID:28857338; Kyoungmi et al. Nature Biotechnology 35:435-437 (2017), the contents of each of which are incorporated herein by reference in their entirety) or an adenine/thymine “A/T” nucleotide pair to a guanine/cytosine “G/C” nucleotide pair (see e.g., Gaudelli et al. Nature (2017), in press doi:10.1038/nature24644, the contents of which are incorporated herein by reference in its entirety).

As used herein, the term “genomic safe harbor gene” or “safe harbor gene” refers to a gene or loci that a nucleic acid sequence can be inserted such that the sequence can integrate and function in a predictable manner (e.g., express a protein of interest) without significant negative consequences to endogenous gene activity, or the promotion of cancer. In some embodiments, a safe harbor gene is also a loci or gene where an inserted nucleic acid sequence can be expressed efficiently and at higher levels than a non-safe harbor site.

As used herein, the term “gene delivery” means a process by which foreign DNA is transferred to host cells for applications of gene therapy.

As used herein, the term “CRISPR” stands for Clustered Regularly Interspaced Short Palindromic Repeats, which are the hallmark of a bacterial defense system that forms the basis for CRISPR-Cas9 genome editing technology.

As used herein, the term “zinc finger” means a small protein structural motif that is characterized by the coordination of one or more zinc ions, in order to stabilize the fold.

As used herein, the term “homologous recombination” means a type of genetic recombination in which nucleotide sequences are exchanged between two similar or identical molecules of DNA. Homologous recombination also produces new combinations of DNA sequences. These new combinations of DNA represent genetic variation. Homologous recombination is also used in horizontal gene transfer to exchange genetic material between different strains and species of viruses.

As used herein, the term “terminal repeat” or “TR” includes any viral terminal repeat or synthetic sequence that comprises at least one minimal required origin of replication and a region comprising a palindrome hairpin structure. A Rep-binding sequence (“RBS”) (also referred to as RBE (Rep-binding element)) and a terminal resolution site (“TRS”) together constitute a “minimal required origin of replication” and thus the TR comprises at least one RBS and at least one TRS. TRs that are the inverse complement of one another within a given stretch of polynucleotide sequence are typically each referred to as an “inverted terminal repeat” or “ITR”. In the context of a virus, ITRs mediate replication, virus packaging, integration and provirus rescue. As was unexpectedly found in the invention herein, TRs that are not inverse complements across their full length can still perform the traditional functions of ITRs, and thus the term ITR is used herein to refer to a TR in a cleavable ceDNA genome or ceDNA vector that is capable of mediating replication of ceDNA vector. It will be understood by one of ordinary skill in the art that in complex ceDNA vector configurations more than two ITRs or asymmetric ITR pairs may be present. The ITR can be an AAV ITR or a non-AAV ITR, or can be derived from an AAV ITR or a non-AAV ITR. For example, the ITR can be derived from the family Parvoviridae, which encompasses parvoviruses and dependoviruses (e.g., canine parvovirus, bovine parvovirus, mouse parvovirus, porcine parvovirus, human parvovirus B-19), or the SV40 hairpin that serves as the origin of SV40 replication can be used as an ITR, which can further be modified by truncation, substitution, deletion, insertion and/or addition. Parvoviridae family viruses consist of two subfamilies: Parvovirinae, which infect vertebrates, and Densovirinae, which infect invertebrates. Dependoparvoviruses include the viral family of the adeno-associated viruses (AAV) which are capable of replication in vertebrate hosts including, but not limited to, human, primate, bovine, canine, equine and ovine species. For convenience herein, an ITR located 5′ to (upstream of) an expression cassette in a cleavable ceDNA vector is referred to as a “5′ ITR” or a “left ITR”, and an ITR located 3′ to (downstream of) an expression cassette in a cleavable ceDNA vector is referred to as a “3′ ITR” or a “right ITR”.

A “wild-type ITR” or “WT-ITR” refers to the sequence of a naturally occurring ITR sequence in an AAV or other dependovirus that retains, e.g., Rep binding activity and Rep nicking ability. The nucleotide sequence of a WT-ITR from any AAV serotype may slightly vary from the canonical naturally occurring sequence due to degeneracy of the genetic code or drift, and therefore WT-ITR sequences encompassed for use herein include WT-ITR sequences as result of naturally occurring changes taking place during the production process (e.g., a replication error).

As used herein, the term “substantially symmetrical WT-ITRs” or a “substantially symmetrical WT-ITR pair” refers to a pair of WT-ITRs within a single ceDNA genome or ceDNA vector that are both wild type ITRs that have an inverse complement sequence across their entire length. For example, an ITR can be considered to be a wild-type sequence, even if it has one or more nucleotides that deviate from the canonical naturally occurring sequence, so long as the changes do not affect the properties and overall three-dimensional structure of the sequence. In some aspects, the deviating nucleotides represent conservative sequence changes. As one non-limiting example, a sequence that has at least 95%, 96%, 97%, 98%, or 99% sequence identity to the canonical sequence (as measured, e.g., using BLAST at default settings), and also has a symmetrical three-dimensional spatial organization to the other WT-ITR such that their 3D structures are the same shape in geometrical space. The substantially symmetrical WT-ITR has the same A, C-C′ and B-B′ loops in 3D space. A substantially symmetrical WT-ITR can be functionally confirmed as WT by determining that it has an operable Rep binding site (RBE or RBE′) and terminal resolution site (trs) that pairs with the appropriate Rep protein. One can optionally test other functions, including transgene expression under permissive conditions.

As used herein, the phrases of “modified ITR” or “mod-ITR” or “mutant ITR” are used interchangeably herein and refer to an ITR that has a mutation in at least one or more nucleotides as compared to the WT-ITR from the same serotype. The mutation can result in a change in one or more of A, C, C′, B, B′ regions in the ITR, and can result in a change in the three-dimensional spatial organization (i.e. its 3D structure in geometric space) as compared to the 3D spatial organization of a WT-ITR of the same serotype.

As used herein, the term “asymmetric ITRs” also referred to as “asymmetric ITR pairs” refers to a pair of ITRs within a single ceDNA genome or ceDNA vector that are not inverse complements across their full length. As one non-limiting example, an asymmetric ITR pair does not have a symmetrical three-dimensional spatial organization to their cognate ITR such that their 3D structures are different shapes in geometrical space. Stated differently, an asymmetrical ITR pair have the different overall geometric structure, i.e., they have different organization of their A, C-C′ and B-B′ loops in 3D space (e.g., one ITR may have a short C-C′ arm and/or short B-B′ arm as compared to the cognate ITR). The difference in sequence between the two ITRs may be due to one or more nucleotide addition, deletion, truncation, or point mutation. In one embodiment, one ITR of the asymmetric ITR pair may be a wild-type AAV ITR sequence and the other ITR a modified ITR as defined herein (e.g., a non-wild-type or synthetic ITR sequence). In another embodiment, neither ITRs of the asymmetric ITR pair is a wild-type AAV sequence and the two ITRs are modified ITRs that have different shapes in geometrical space (i.e., a different overall geometric structure). In some embodiments, one mod-ITRs of an asymmetric ITR pair can have a short C-C′ arm and the other ITR can have a different modification (e.g., a single arm, or a short B-B′ arm etc.) such that they have different three-dimensional spatial organization as compared to the cognate asymmetric mod-ITR

As used herein, the term “symmetric ITRs” refers to a pair of ITRs within a single ceDNA genome or ceDNA vector that are mutated or modified relative to wild-type dependoviral ITR sequences and are inverse complements across their full length. Neither ITRs are wild type ITR AAV2 sequences (i.e., they are a modified ITR, also referred to as a mutant ITR), and can have a difference in sequence from the wild type ITR due to nucleotide addition, deletion, substitution, truncation, or point mutation. For convenience herein, an ITR located 5′ to (upstream of) an expression cassette in a cleavable ceDNA vector is referred to as a “5′ ITR” or a “left ITR”, and an ITR located 3′ to (downstream of) an expression cassette in a cleavable ceDNA vector is referred to as a “3′ ITR” or a “right ITR”.

As used herein, the terms “substantially symmetrical modified-ITRs” or a “substantially symmetrical mod-ITR pair” refers to a pair of modified-ITRs within a single ceDNA genome or ceDNA vector that are both that have an inverse complement sequence across their entire length. For example, the a modified ITR can be considered substantially symmetrical, even if it has some nucleotide sequences that deviate from the inverse complement sequence so long as the changes do not affect the properties and overall shape. As one non-limiting example, a sequence that has at least 85%, 90%, 95%, 96%, 97%, 98%, or 99% sequence identity to the canonical sequence (as measured using BLAST at default settings), and also has a symmetrical three-dimensional spatial organization to their cognate modified ITR such that their 3D structures are the same shape in geometrical space. Stated differently, a substantially symmetrical modified-ITR pair have the same A, C-C′ and B-B′ loops organized in 3D space. In some embodiments, the ITRs from a mod-ITR pair may have different reverse complement nucleotide sequences but still have the same symmetrical three-dimensional spatial organization—that is both ITRs have mutations that result in the same overall 3D shape. For example, one ITR (e.g., 5′ ITR) in a mod-ITR pair can be from one serotype, and the other ITR (e.g., 3′ ITR) can be from a different serotype, however, both can have the same corresponding mutation (e.g., if the 5′ITR has a deletion in the C region, the cognate modified 3′ITR from a different serotype has a deletion at the corresponding position in the C′ region), such that the modified ITR pair has the same symmetrical three-dimensional spatial organization. In such embodiments, each ITR in a modified ITR pair can be from different serotypes (e.g., AAV1, 2, 3, 4, 5, 6, 7, 8, 9, 10, 11, and 12) such as the combination of AAV2 and AAV6, with the modification in one ITR reflected in the corresponding position in the cognate ITR from a different serotype. In one embodiment, a substantially symmetrical modified ITR pair refers to a pair of modified ITRs (mod-ITRs) so long as the difference in nucleotide sequences between the ITRs does not affect the properties or overall shape and they have substantially the same shape in 3D space. As a non-limiting example, a mod-ITR that has at least 95%, 96%, 97%, 98% or 99% sequence identity to the canonical mod-ITR as determined by standard means well known in the art such as BLAST (Basic Local Alignment Search Tool), or BLASTN at default settings, and also has a symmetrical three-dimensional spatial organization such that their 3D structure is the same shape in geometric space. A substantially symmetrical mod-ITR pair has the same A, C-C′ and B-B′ loops in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A and B-B′ loops in the same shape in geometric space of its cognate mod-ITR.

The term “flanking” refers to a relative position of one nucleic acid sequence with respect to another nucleic acid sequence. Generally, in the sequence ABC, B is flanked by A and C. The same is true for the arrangement A×B×C. Thus, a flanking sequence precedes or follows a flanked sequence but need not be contiguous with, or immediately adjacent to the flanked sequence. In one embodiment, the term flanking refers to terminal repeats at each end of the linear duplex ceDNA vector.

As used herein, the term “ceDNA genome” refers to an expression cassette that further incorporates at least one inverted terminal repeat region. A cleavable ceDNA genome may further comprise one or more spacer regions. In some embodiments the cleavable ceDNA genome is incorporated as an intermolecular duplex polynucleotide of DNA into a plasmid or viral genome.

As used herein, the term “ceDNA spacer region” refers to an intervening sequence that separates functional elements in the cleavable ceDNA vector or ceDNA genome. In some embodiments, ceDNA spacer regions keep two functional elements at a desired distance for optimal functionality. In some embodiments, ceDNA spacer regions provide or add to the genetic stability of the cleavable ceDNA genome within e.g., a plasmid or baculovirus. In some embodiments, ceDNA spacer regions facilitate ready genetic manipulation of the cleavable ceDNA genome by providing a convenient location for cloning sites and the like. For example, in certain aspects, an oligonucleotide “polylinker” containing several restriction endonuclease sites, or a non-open reading frame sequence designed to have no known protein (e.g., transcription factor) binding sites can be positioned in the cleavable ceDNA genome to separate the cis—acting factors, e.g., inserting a 6 mer, 12 mer, 18 mer, 24 mer, 48 mer, 86 mer, 176 mer, etc. between the terminal resolution site and the upstream transcriptional regulatory element. Similarly, the ceDNA spacer may be incorporated between the polyadenylation signal sequence and the 3′-terminal resolution site.

As used herein, the terms “Rep binding site, “Rep binding element, “RBE” and “RBS” are used interchangeably and refer to a binding site for Rep protein (e.g., AAV Rep 78 or AAV Rep 68) which upon binding by a Rep protein permits the Rep protein to perform its site-specific endonuclease activity on the sequence incorporating the RBS. An RBS sequence and its inverse complement together form a single RBS. RBS sequences are known in the art, and include, for example, 5′-GCGCGCTCGCTCGCTC-3′ (SEQ ID NO: 1), an RBS sequence identified in AAV2. Any known RBS sequence may be used in the embodiments of the invention, including other known AAV RBS sequences and other naturally known or synthetic RBS sequences. Without being bound by theory it is thought that he nuclease domain of a Rep protein binds to the duplex nucleotide sequence GCTC, and thus the two known AAV Rep proteins bind directly to and stably assemble on the duplex oligonucleotide, 5′-(GCGC)(GCTC)(GCTC)(GCTC)-3′ (SEQ ID NO: 2). In addition, soluble aggregated conformers (i.e., undefined number of inter-associated Rep proteins) dissociate and bind to oligonucleotides that contain Rep binding sites. Each Rep protein interacts with both the nitrogenous bases and phosphodiester backbone on each strand. The interactions with the nitrogenous bases provide sequence specificity whereas the interactions with the phosphodiester backbone are non- or less-sequence specific and stabilize the protein-DNA complex.

As used herein, the terms “terminal resolution site” and “TRS” are used interchangeably herein and refer to a region at which Rep forms a tyrosine-phosphodiester bond with the 5′ thymidine generating a 3′ OH that serves as a substrate for DNA extension via a cellular DNA polymerase, e.g., DNA pol delta or DNA pol epsilon. Alternatively, the Rep-thymidine complex may participate in a coordinated ligation reaction. In some embodiments, a TRS minimally encompasses a non-base-paired thymidine. In some embodiments, the nicking efficiency of the TRS can be controlled at least in part by its distance within the same molecule from the RBS. When the acceptor substrate is the complementary ITR, then the resulting product is an intramolecular duplex. TRS sequences are known in the art, and include, for example, 5′-GGTTGA-3′, the hexanucleotide sequence identified in AAV2. Any known TRS sequence may be used in the embodiments of the invention, including other known AAV TRS sequences and other naturally known or synthetic TRS sequences such as AGTT, GGTTGG, AGTTGG, AGTTGA, and other motifs such as RRTTRR.

As used herein, the term “ceDNA-plasmid” refers to a plasmid that comprises a cleavable ceDNA genome as an intermolecular duplex.

As used herein, the term “ceDNA-bacmid” refers to an infectious baculovirus genome comprising a cleavable ceDNA genome as an intermolecular duplex that is capable of propagating in E. coli as a plasmid, and so can operate as a shuttle vector for baculovirus.

As used herein, the term “ceDNA-baculovirus” refers to a baculovirus that comprises a cleavable ceDNA genome as an intermolecular duplex within the baculovirus genome.

As used herein, the terms “ceDNA-baculovirus infected insect cell” and “ceDNA-BIIC” are used interchangeably, and refer to an invertebrate host cell (including, but not limited to an insect cell (e.g., an Sf9 cell)) infected with a cleavable ceDNA-baculovirus.

As defined herein, “reporters” refer to proteins that can be used to provide detectable read-outs. Reporters generally produce a measurable signal such as fluorescence, color, or luminescence. Reporter protein coding sequences encode proteins whose presence in the cell or organism is readily observed. For example, fluorescent proteins cause a cell to fluoresce when excited with light of a particular wavelength, luciferases cause a cell to catalyze a reaction that produces light, and enzymes such as β-galactosidase convert a substrate to a colored product. Exemplary reporter polypeptides useful for experimental or diagnostic purposes include, but are not limited to β-lactamase, β-galactosidase (LacZ), alkaline phosphatase (AP), thymidine kinase (TK), green fluorescent protein (GFP) and other fluorescent proteins, chloramphenicol acetyltransferase (CAT), luciferase, and others well known in the art.

As used herein, the term “effector protein” refers to a polypeptide that provides a detectable read-out, either as, for example, a reporter polypeptide, or more appropriately, as a polypeptide that kills a cell, e.g., a toxin, or an agent that renders a cell susceptible to killing with a chosen agent or lack thereof. Effector proteins include any protein or peptide that directly targets or damages the host cell's DNA and/or RNA. For example, effector proteins can include, but are not limited to, a restriction endonuclease that targets a host cell DNA sequence (whether genomic or on an extrachromosomal element), a protease that degrades a polypeptide target necessary for cell survival, a DNA gyrase inhibitor, and a ribonuclease-type toxin. In some embodiments, the expression of an effector protein controlled by a synthetic biological circuit as described herein can participate as a factor in another synthetic biological circuit to thereby expand the range and complexity of a biological circuit system's responsiveness.

Transcriptional regulators refer to transcriptional activators and repressors that either activate or repress transcription of a gene of interest. Promoters are regions of nucleic acid that initiate transcription of a particular gene Transcriptional activators typically bind nearby to transcriptional promoters and recruit RNA polymerase to directly initiate transcription. Repressors bind to transcriptional promoters and sterically hinder transcriptional initiation by RNA polymerase. Other transcriptional regulators may serve as either an activator or a repressor depending on where they bind and cellular and environmental conditions. Non-limiting examples of transcriptional regulator classes include, but are not limited to homeodomain proteins, zinc-finger proteins, winged-helix (forkhead) proteins, and leucine-zipper proteins.

As used herein, a “repressor protein” or “inducer protein” is a protein that binds to a regulatory sequence element and represses or activates, respectively, the transcription of sequences operatively linked to the regulatory sequence element. Preferred repressor and inducer proteins as described herein are sensitive to the presence or absence of at least one input agent or environmental input. Preferred proteins as described herein are modular in form, comprising, for example, separable DNA-binding and input agent-binding or responsive elements or domains.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Supplementary active ingredients can also be incorporated into the compositions. The phrase “pharmaceutically-acceptable” refers to molecular entities and compositions that do not produce a toxic, an allergic, or similar untoward reaction when administered to a host.

As used herein, an “input agent responsive domain” is a domain of a transcription factor that binds to or otherwise responds to a condition or input agent in a manner that renders a linked DNA binding fusion domain responsive to the presence of that condition or input. In one embodiment, the presence of the condition or input results in a conformational change in the input agent responsive domain, or in a protein to which it is fused, that modifies the transcription-modulating activity of the transcription factor.

The term “in vivo” refers to assays or processes that occur in or within an organism, such as a multicellular animal. In some of the aspects described herein, a method or use can be said to occur “in vivo” when a unicellular organism, such as a bacterium, is used. The term “ex vivo” refers to methods and uses that are performed using a living cell with an intact membrane that is outside of the body of a multicellular animal or plant, e.g., explants, cultured cells, including primary cells and cell lines, transformed cell lines, and extracted tissue or cells, including blood cells, among others. The term “in vitro” refers to assays and methods that do not require the presence of a cell with an intact membrane, such as cellular extracts, and can refer to the introducing of a programmable synthetic biological circuit in a non-cellular system, such as a medium not comprising cells or cellular systems, such as cellular extracts.

The term “promoter,” as used herein, refers to any nucleic acid sequence that regulates the expression of another nucleic acid sequence by driving transcription of the nucleic acid sequence, which can be a heterologous target gene encoding a protein or an RNA. Promoters can be constitutive, inducible, repressible, tissue-specific, or any combination thereof. A promoter is a control region of a nucleic acid sequence at which initiation and rate of transcription of the remainder of a nucleic acid sequence are controlled. A promoter can also contain genetic elements at which regulatory proteins and molecules can bind, such as RNA polymerase and other transcription factors. In some embodiments of the aspects described herein, a promoter can drive the expression of a transcription factor that regulates the expression of the promoter itself. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the expression of transgenes in the cleavable ceDNA vectors disclosed herein. A promoter sequence may be bounded at its 3′ terminus by the transcription initiation site and extends upstream (5′ direction) to include the minimum number of bases or elements necessary to initiate transcription at levels detectable above background.

The term “enhancer” as used herein refers to a cis-acting regulatory sequence (e.g., 50-1,500 base pairs) that binds one or more proteins (e.g., activator proteins, or transcription factor) to increase transcriptional activation of a nucleic acid sequence. Enhancers can be positioned up to 1,000,000 base pars upstream of the gene start site or downstream of the gene start site that they regulate. An enhancer can be positioned within an intronic region, or in the exonic region of an unrelated gene.

A promoter can be said to drive expression or drive transcription of the nucleic acid sequence that it regulates. The phrases “operably linked,” “operatively positioned,” “operatively linked,” “under control,” and “under transcriptional control” indicate that a promoter is in a correct functional location and/or orientation in relation to a nucleic acid sequence it regulates to control transcriptional initiation and/or expression of that sequence. An “inverted promoter,” as used herein, refers to a promoter in which the nucleic acid sequence is in the reverse orientation, such that what was the coding strand is now the non-coding strand, and vice versa. Inverted promoter sequences can be used in various embodiments to regulate the state of a switch. In addition, in various embodiments, a promoter can be used in conjunction with an enhancer.

A promoter can be one naturally associated with a gene or sequence, as can be obtained by isolating the 5′ non-coding sequences located upstream of the coding segment and/or exon of a given gene or sequence. Such a promoter can be referred to as “endogenous.” Similarly, in some embodiments, an enhancer can be one naturally associated with a nucleic acid sequence, located either downstream or upstream of that sequence.

In some embodiments, a coding nucleic acid segment is positioned under the control of a “recombinant promoter” or “heterologous promoter,” both of which refer to a promoter that is not normally associated with the encoded nucleic acid sequence it is operably linked to in its natural environment. A recombinant or heterologous enhancer refers to an enhancer not normally associated with a given nucleic acid sequence in its natural environment. Such promoters or enhancers can include promoters or enhancers of other genes; promoters or enhancers isolated from any other prokaryotic, viral, or eukaryotic cell; and synthetic promoters or enhancers that are not “naturally occurring.” i.e., comprise different elements of different transcriptional regulatory regions, and/or mutations that alter expression through methods of genetic engineering that are known in the art. In addition to producing nucleic acid sequences of promoters and enhancers synthetically, promoter sequences can be produced using recombinant cloning and/or nucleic acid amplification technology, including PCR, in connection with the synthetic biological circuits and modules disclosed herein (see, e.g., U.S. Pat. Nos. 4,683,202, 5,928,906, each incorporated herein by reference). Furthermore, it is contemplated that control sequences that direct transcription and/or expression of sequences within non-nuclear organelles such as mitochondria, chloroplasts, and the like, can be employed as well.

As described herein, an “inducible promoter” is one that is characterized by initiating or enhancing transcriptional activity when in the presence of, influenced by, or contacted by an inducer or inducing agent. An “inducer” or “inducing agent,” as defined herein, can be endogenous, or a normally exogenous compound or protein that is administered in such a way as to be active in inducing transcriptional activity from the inducible promoter. In some embodiments, the inducer or inducing agent, i.e., a chemical, a compound or a protein, can itself be the result of transcription or expression of a nucleic acid sequence (i.e., an inducer can be an inducer protein expressed by another component or module), which itself can be under the control or an inducible promoter. In some embodiments, an inducible promoter is induced in the absence of certain agents, such as a repressor. Examples of inducible promoters include but are not limited to, tetracycline, metallothionine, ecdysone, mammalian viruses (e.g., the adenovirus late promoter; and the mouse mammary tumor virus long terminal repeat (MMTV-LTR)) and other steroid-responsive promoters, rapamycin responsive promoters and the like.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., DNA-targeting RNA) or a coding sequence (e.g., site-directed modifying polypeptide, or Cas9/Csn1 polypeptide) and/or regulate translation of an encoded polypeptide.

“Operably linked” refers to a juxtaposition wherein the components so described are in a relationship permitting them to function in their intended manner. For instance, a promoter is operably linked to a coding sequence if the promoter affects its transcription or expression. An “expression cassette” includes an exogenous DNA sequence that is operably linked to a promoter or other regulatory sequence sufficient to direct transcription of the transgene in the cleavable ceDNA vector. Suitable promoters include, for example, tissue specific promoters. Promoters can also be of AAV origin.

The term “subject” as used herein refers to a human or animal, to whom treatment, including prophylactic treatment, with the cleavable ceDNA vector according to the present invention, is provided. Usually the animal is a vertebrate such as, but not limited to a primate, rodent, domestic animal or game animal. Primates include but are not limited to, chimpanzees, cynomolgus monkeys, spider monkeys, and macaques, e.g., Rhesus. Rodents include mice, rats, woodchucks, ferrets, rabbits and hamsters. Domestic and game animals include, but are not limited to, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments of the aspects described herein, the subject is a mammal, e.g., a primate or a human. A subject can be male or female. Additionally, a subject can be an infant or a child. In some embodiments, the subject can be a neonate or an unborn subject, e.g., the subject is in utero. Preferably, the subject is a mammal. The mammal can be a human, non-human primate, mouse, rat, dog, cat, horse, or cow, but is not limited to these examples. Mammals other than humans can be advantageously used as subjects that represent animal models of diseases and disorders. In addition, the methods and compositions described herein can be used for domesticated animals and/or pets. A human subject can be of any age, gender, race or ethnic group, e.g., Caucasian (white), Asian, African, black, African American, African European, Hispanic, Mideastern, etc. In some embodiments, the subject can be a patient or other subject in a clinical setting. In some embodiments, the subject is already undergoing treatment. In some embodiments, the subject is an embryo, a fetus, neonate, infant, child, adolescent, or adult. In some embodiments, the subject is a human fetus, human neonate, human infant, human child, human adolescent, or human adult. In some embodiments, the subject is an animal embryo, or non-human embryo or non-human primate embryo. In some embodiments, the subject is a human embryo.

As used herein, the term “host cell”, includes any cell type that is susceptible to transformation, transfection, transduction, and the like with a nucleic acid construct or ceDNA expression vector of the present disclosure. As non-limiting examples, a host cell can be an isolated primary cell, pluripotent stem cells, CD34⁺ cells), induced pluripotent stem cells, or any of a number of immortalized cell lines (e.g., HepG2 cells). Alternatively, a host cell can be an in situ or in vivo cell in a tissue, organ or organism.

The term “exogenous” refers to a substance present in a cell other than its native source. The term “exogenous” when used herein can refer to a nucleic acid (e.g., a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell.

The term “sequence identity” refers to the relatedness between two nucleotide sequences. For purposes of the present disclosure, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows: (identical Deoxyribonucleotides.times.100)/(Length of Alignment-Total Number of Gaps in Alignment). The length of the alignment is preferably at least 10 nucleotides, preferably at least 25 nucleotides more preferred at least 50 nucleotides and most preferred at least 100 nucleotides.

The term “homology” or “homologous” as used herein is defined as the percentage of nucleotide residues in the homology arm that are identical to the nucleotide residues in the corresponding sequence on the target chromosome, after aligning the sequences and introducing gaps, if necessary, to achieve the maximum percent sequence identity. Alignment for purposes of determining percent nucleotide sequence homology can be achieved in various ways that are within the skill in the art, for instance, using publicly available computer software such as BLAST, BLAST-2, ALIGN, ClustalW2 or Megalign (DNASTAR) software. Those skilled in the art can determine appropriate parameters for aligning sequences, including any algorithms needed to achieve maximal alignment over the full length of the sequences being compared. In some embodiments, a nucleic acid sequence (e.g., DNA sequence), for example of a homology arm of a repair template, is considered “homologous” when the sequence is at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to the corresponding native or unedited nucleic acid sequence (e.g., genomic sequence) of the host cell.

As used herein, a “homology arm” refers to a polynucleotide that is suitable to target a donor sequence to a genome through homologous recombination. Typically, two homology arms flank the donor sequence, wherein each homology arm comprises genomic sequences upstream and downstream of the loci of integration.

As used herein, “a donor sequence” refers to a polynucleotide that is to be inserted into, or used as a repair template for, a host cell genome. The donor sequence can comprise the modification which is desired to be made during gene editing. The sequence to be incorporated can be introduced into the target nucleic acid molecule via homology directed recombination at the target sequence, thereby causing an alteration of the target sequence from the original target sequence to the sequence comprised by the donor sequence. Accordingly, the sequence comprised by the donor sequence can be, relative to the target sequence, an insertion, a deletion, an indel, a point mutation, a repair of a mutation, etc. The donor sequence can be, e.g., a single-stranded DNA molecule; a double-stranded DNA molecule; a DNA/RNA hybrid molecule; and a DNA/modRNA (modified RNA) hybrid molecule. In one embodiment, the donor sequence is foreign to the homology arms. The editing can be RNA as well as DNA editing. The donor sequence can be endogenous to or exogenous to the host cell genome, depending upon the nature of the desired gene editing. The cleaved ceDNA of the instant disclosure may act as a donor sequence.

The term “heterologous,” as used herein, means a nucleotide or polypeptide sequence that is not found in the native nucleic acid or protein, respectively. For example, in a chimeric Cas9/Csn1 protein, the RNA-binding domain of a naturally-occurring bacterial Cas9/Csn1 polypeptide (or a variant thereof) may be fused to a heterologous polypeptide sequence (i.e. a polypeptide sequence from a protein other than Cas9/Csn1 or a polypeptide sequence from another organism). The heterologous polypeptide sequence may exhibit an activity (e.g., enzymatic activity) that will also be exhibited by the chimeric Cas9/Csn1 protein (e.g., methyltransferase activity, acetyltransferase activity, kinase activity, ubiquitinating activity, etc.). A heterologous nucleic acid sequence may be linked to a naturally-occurring nucleic acid sequence (or a variant thereof) (e.g., by genetic engineering) to generate a chimeric nucleotide sequence encoding a chimeric polypeptide. As another example, in a fusion variant Cas9 site-directed polypeptide, a variant Cas9 site-directed polypeptide may be fused to a heterologous polypeptide (i.e. a polypeptide other than Cas9), which exhibits an activity that will also be exhibited by the fusion variant Cas9 site-directed polypeptide. A heterologous nucleic acid sequence may be linked to a variant Cas9 site-directed polypeptide (e.g., by genetic engineering) to generate a nucleotide sequence encoding a fusion variant Cas9 site-directed polypeptide.

A “vector” or “expression vector” is a replicon, such as plasmid, bacmid, phage, virus, virion, or cosmid, to which another DNA segment, i.e. an “insert”, may be attached so as to bring about the replication of the attached segment in a cell. A vector can be a nucleic acid construct designed for delivery to a host cell or for transfer between different host cells. As used herein, a vector can be viral or non-viral in origin and/or in final form, however for the purpose of the present disclosure, a “vector” generally refers to a ceDNA vector, as that term is used herein. The term “vector” encompasses any genetic element that is capable of replication when associated with the proper control elements and that can transfer gene sequences to cells. In some embodiments, a vector can be an expression vector or recombinant vector.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

The terms “correcting”, “genome editing” and “restoring” as used herein refers to changing a mutant gene that encodes a truncated protein or no protein at all, such that a full-length functional or partially full-length functional protein expression is obtained. Correcting or restoring a mutant gene may include replacing the region of the gene that has the mutation or replacing the entire mutant gene with a copy of the gene that does not have the mutation with a repair mechanism such as homology-directed recombination (HDR). Correcting or restoring a mutant gene may also include repairing a frameshift mutation that causes a premature stop codon, an aberrant splice acceptor site or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene may also include disrupting an aberrant splice acceptor site or splice donor sequence. Correcting or restoring a mutant gene may also include deleting a non-essential gene segment by the simultaneous action of two nucleases on the same DNA strand in order to restore the proper reading frame by removing the DNA between the two nuclease target sites and repairing the DNA break by NHEJ.

The phrase “genetic disease” as used herein refers to a disease, partially or completely, directly or indirectly, caused by one or more abnormalities in the genome, especially a condition that is present from birth. The abnormality may be a mutation, an insertion or a deletion. The abnormality may affect the coding sequence of the gene or its regulatory sequence. According to some embodiments, the genetic disease may be, but not limited to DMD, hemophilia, cystic fibrosis, Huntington's chorea, familial hypercholesterolemia (LDL receptor defect), hepatoblastoma, Wilson's disease, congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, and Tay-Sachs disease.

The phrase “repeat variable diresidue” or “RVD” as used interchangeably herein refers to a pair of adjacent amino acid residues within a DNA recognition motif (also known as “RVD module”), which includes 33-35 amino acids, of a TALE DNA-binding domain. The RVD determines the nucleotide specificity of the RVD module. RVD modules may be combined to produce an RVD array. The “RVD array length” as used herein refers to the number of RVD modules that corresponds to the length of the nucleotide sequence within the TALEN target region that is recognized by a TALEN, i.e., the binding region.

The terms “site-specific nuclease” or “sequence specific nuclease” as used herein refers to an enzyme capable of specifically recognizing and cleaving DNA sequences. The site-specific nuclease may be engineered. Examples of engineered site-specific nucleases include zinc finger nucleases (ZFNs), TAL effector nucleases (TALENs), and CRISPR/Cas-based systems, that use various natural and unnatural Cas enzymes.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Other than in the operating examples, or where otherwise indicated, all numbers expressing quantities of ingredients or reaction conditions used herein should be understood as modified in all instances by the term “about.” The term “about” when used in connection with percentages can mean±1%. The present invention is further explained in detail by the following examples, but the scope of the invention should not be limited thereto.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### II. Cleavable Closed-Ended DNAs

Embodiments of the invention are based on methods and compositions comprising cleavable closed-ended linear duplexed (ceDNA). After cleavage of a cleavable ceDNA by a Cas enzyme using a gRNA which binds to a gRNA target sequence (TS) in the cleavable ceDNA, the resulting “cleaved ceDNA” may be used as a donor sequence for gene editing purposes. According to some embodiments, the cleaved ceDNA comprises a transgene cassette. In one embodiment, a “cleaved ceDNA” does not comprise an ITR.

One aspect herein relates to a novel cleaved ceDNA vector for DNA knock-in method(s), e.g., for the introduction of one or more exogenous donor sequences into a specific target site on a cellular chromosome with high efficiency. The cleavable ceDNA described herein may comprise ITR sequences selected from any of: (i) at least one WT ITR and at least one modified AAV inverted terminal repeat (mod-ITR) (e.g., asymmetric modified ITRs): (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization, the methods and compositions as disclosed herein may further include a delivery system, such as but not limited to, a liposome nanoparticle delivery system. Nonlimiting exemplary liposome nanoparticle systems encompassed for use are disclosed herein. In some aspects, the disclosure provides for a lipid nanoparticle comprising a ceDNA and an ionizable lipid. For example, a lipid nanoparticle formulation that is made and loaded with a ceDNA obtained by the process is disclosed in International Application PCT/US2018/050042, filed on Sep. 7, 2018, which is incorporated herein.

Provided herein are novel non-viral, capsid-free cleavable ceDNA molecules with covalently-closed ends (ceDNA). These non-viral capsid free ceDNA molecules can be produced in permissive host cells from an expression construct (e.g., a cleavable ceDNA-plasmid, a cleavable ceDNA-bacmid, a cleavable ceDNA-baculovirus, or an integrated cell-line) containing a heterologous gene (transgene) positioned between two different inverted terminal repeat (ITR) sequences, where the ITRs are different with respect to each other. In some embodiments, one of the ITRs is modified by deletion, insertion, and/or substitution as compared to a wild-type ITR sequence (e.g., AAV ITR); and at least one of the ITRs comprises a functional terminal resolution site (trs) and a Rep binding site. The cleavable ceDNA vector is preferably duplex, e.g., self-complementary, over at least a portion of the molecule, such as the expression cassette (e.g., ceDNA is not a double stranded circular molecule). The cleavable ceDNA vector has covalently closed ends, and thus is resistant to exonuclease digestion (e.g., exonuclease I or exonuclease III), e.g., for over an hour at 37° C.

The cleavable ceDNA vectors as disclosed herein have no packaging constraints imposed by the limiting space within the viral capsid. Cleavable ceDNA vectors represent a viable eukaryotically-produced alternative to prokaryote-produced plasmid DNA vectors, as opposed to encapsulated AAV genomes. This permits the insertion of control elements, e.g., regulatory switches as disclosed herein, large transgenes, multiple transgenes etc.

In one aspect, a cleavable ceDNA comprises, in the following order, a first inverted terminal repeat (ITR); a first guide RNA (gRNA) target sequence (TS); a first protospacer adjacent motif (PAM); at least one transgene cassette; a second protospacer adjacent motif (PAM); a second gRNA target sequence (TS); and a second ITR.

In some embodiments, the first ITR (5′ ITR) and the second ITR (3′ ITR) are asymmetric with respect to each other—that is, they have a different 3D-spatial configuration from one another. As an exemplary embodiment, the first ITR can be a wild-type ITR and the second ITR can be a mutated or modified ITR, or vice versa, where the first ITR can be a mutated or modified ITR and the second ITR a wild-type ITR. In another embodiment, the first ITR and the second ITR are both modified but are different sequences, or have different modifications, or are not identical modified ITRs, and have different 3D spatial configurations. Stated differently, a cleavable ceDNA with asymmetric ITRs have ITRs where any changes in one ITR relative to the WT-ITR are not reflected in the other ITR; or alternatively, where the asymmetric ITRs have a the modified asymmetric ITR pair can have a different sequence and different three-dimensional shape with respect to each other. Exemplary asymmetric ITRs are discussed below in the section entitled “asymmetric ITRs”.

In another aspect, a cleavable ceDNA comprises, in the following order, a first inverted terminal repeat (ITR); a first guide RNA (gRNA) target sequence (TS); a first protospacer adjacent motif (PAM); at least one transgene cassette; a second protospacer adjacent motif (PAM); a second gRNA target sequence (TS); and a second ITR, where the first ITR (5′ ITR) and the second ITR (3′ ITR) are symmetric, or substantially symmetrical with respect to each other—that is, a ceDNA vector can comprise ITR sequences that have a symmetrical three-dimensional spatial organization such that their structure is the same shape in geometrical space, or have the same A, C-C′ and B-B′ loops in 3D space. In such an embodiment, a symmetrical ITR pair, or substantially symmetrical ITR pair can be modified ITRs (e.g., mod-ITRs) that are not wild-type ITRs. A mod-ITR pair can have the same sequence which has one or more modifications from wild-type ITR and are reverse complements (inverted) of each other. In alternative embodiments, a modified ITR pair are substantially symmetrical as defined herein, that is, the modified ITR pair can have a different sequence but have corresponding or the same symmetrical three-dimensional shape. In some embodiments, the symmetrical ITRs, or substantially symmetrical ITRs are wild type (WT-ITRs) as described herein. That is, both ITRs have a wild type sequence, but do not necessarily have to be WT-ITRs from the same AAV serotype. That is, in some embodiments, one WT-ITR can be from one AAV serotype, and the other WT-ITR can be from a different AAV serotype. In such an embodiment, a WT-ITR pair are substantially symmetrical as defined herein, that is, they can have one or more conservative nucleotide modification while still retaining the symmetrical three-dimensional spatial organization.

The symmetric ITRs or substantially symmetrical ITRs are discussed in the section below entitled “symmetrical ITR pairs”.

The wild-type or mutated or otherwise modified ITR sequences provided herein represent DNA sequences included in the expression construct (e.g., ceDNA-plasmid, ceDNA Bacmid, ceDNA-baculovirus) for production of the cleavable ceDNA vector. Thus, ITR sequences actually contained in the cleavable ceDNA vector produced from the cleavable ceDNA-plasmid or other expression construct may or may not be identical to the ITR sequences provided herein as a result of naturally occurring changes taking place during the production process (e.g., replication error).

In some embodiments, a cleavable ceDNA vector described herein may comprise a transgene cassette with a transgene that may or may not be operatively linked to one or more regulatory sequence(s) that allows or controls expression of the transgene. In one embodiment, the polynucleotide comprises a first ITR sequence and a second ITR sequence, wherein the first and second ITR sequences are asymmetrical relative to each other, or symmetrical relative to each other.

In one embodiment in each of these aspects, a transgene cassette is located between a first protospacer adjacent motif (PAM) and a second protospacer adjacent motif (PAM), with one or more of: a promoter operably linked to a transgene, a posttranscriptional regulatory element, and a polyadenylation and termination signal. In one embodiment, the promoter is regulatable—inducible or repressible. The promoter can be any sequence that facilitates the transcription of the transgene. In one embodiment the promoter is a CAG promoter (e.g., SEQ ID NO: 8), or variation thereof.

The posttranscriptional regulatory element is a sequence that modulates expression of the transgene, as a non-limiting example, any sequence that creates a tertiary structure that enhances expression of the transgene.

In one embodiment, the posttranscriptional regulatory element comprises WPRE. In one embodiment, the polyadenylation and termination signal comprises BGHpolyA. Any cis regulatory element known in the art, or combination thereof, can be additionally used e.g., SV40 late polyA signal upstream enhancer sequence (USE), or other posttranscriptional processing elements including, but not limited to, the thymidine kinase gene of herpes simplex virus, or hepatitis B virus (HBV). In one embodiment, the expression cassette length in the 5′ to 3′ direction is greater than the maximum length known to be encapsidated in an AAV virion. In one embodiment, the length is greater than 4.6 kb, or greater than 5 kb, or greater than 6 kb, or greater than 7 kb. Various expression cassettes are exemplified herein.

The expression cassette can comprise more than 4000 nucleotides, 5000 nucleotides, 10,000 nucleotides or 20,000 nucleotides, or 30,000 nucleotides, or 40,000 nucleotides or 50,000 nucleotides, or any range between about 4000-10,000 nucleotides or 10,000-50,000 nucleotides, or more than 50,000 nucleotides. In some embodiments, the expression cassette can comprise a nucleic acid sequence in the range of 500 to 50,000 nucleotides in length. In some embodiments, the expression cassette can comprise a nucleic acid sequence in the range of 500 to 75,000 nucleotides in length. In some embodiments, the expression cassette can comprise a nucleic acid sequence is in the range of 500 to 10,000 nucleotides in length. In some embodiments, the expression cassette can comprise a nucleic acid sequence in the range of 1000 to 10,000 nucleotides in length. In some embodiments, the expression cassette can comprise a nucleic acid sequence in the range of 500 to 5,000 nucleotides in length. The cleavable ceDNA vectors do not have the size limitations of encapsidated AAV vectors, thus enable delivery of a large-size transgene cassettes. In some embodiments, the cleavable ceDNA vector is devoid of prokaryote-specific methylation.

The expression cassette can also comprise an internal ribosome entry site (IRES) and/or a 2A element. The cis-regulatory elements include, but are not limited to, a promoter, a riboswitch, an insulator, a mir-regulatable element, a post-transcriptional regulatory element, a tissue- and cell type-specific promoter and an enhancer. In some embodiments the ITR can act as the promoter for the transgene. In some embodiments, the cleavable ceDNA vector comprises additional components to regulate expression of the transgene, for example, a regulatory switch, which is described herein in the section entitled “Regulatory Switches” for controlling and regulating the expression of the transgene, and can include if desired, a regulatory switch which is a kill switch to enable controlled cell death of a cell comprising a cleavable ceDNA vector.

The cleavable ceDNA vector can comprise a transgene cassette used to edit any gene of interest in the subject, which includes but are not limited to, nucleic acids encoding polypeptides, or non-coding nucleic acids (e.g., RNAi, miRs, etc.), as well as exogenous genes and nucleotide sequences, including virus sequences in a subjects' genome, e.g., HIV virus sequences and the like. Preferably the ceDNA disclosed herein is used for therapeutic purposes (e.g., for medical, diagnostic, or veterinary uses) or immunogenic polypeptides. In certain embodiments, the cleaved ceDNA can be used as a donor sequence to edit any gene of interest in the subject, which includes one or more polypeptides, peptides, ribozymes, peptide nucleic acids, siRNAs, RNAis, antisense oligonucleotides, antisense polynucleotides, antibodies, antigen binding fragments, or any combination thereof.

A cleavable ceDNA as described herein, comprises at least one transgene cassette, wherein the expression cassette can include, for example, an expressible exogenous sequence (e.g., open reading frame) that encodes a protein that is either absent, inactive, or insufficient activity in the recipient subject or a gene that encodes a protein having a desired biological or a therapeutic effect. The exogenous sequence such as a donor sequence can encode a gene product that can function to correct the expression of a defective gene or transcript. The expression cassette can also encode corrective DNA strands, encode polypeptides, sense or antisense oligonucleotides, or RNAs (coding or non-coding; e.g., siRNAs, shRNAs, micro-RNAs, and their antisense counterparts (e.g., antagoMiR)). Expression cassettes can include an exogenous sequence that encodes a reporter protein to be used for experimental or diagnostic purposes, such as β-lactamase, β-galactosidase (LacZ), alkaline phosphatase, thymidine kinase, green fluorescent protein (GFP), chloramphenicol acetyltransferase (CAT), luciferase, and others well known in the art.

In principle, the expression cassette can include any gene that encodes a protein, polypeptide or RNA that is either reduced or absent due to a mutation or which conveys a therapeutic benefit when overexpressed is considered to be within the scope of the disclosure.

Sequences provided in the expression cassette, expression construct, or donor sequence of a cleavable ceDNA vector described herein can be codon optimized for the host cell. As used herein, the term “codon optimized” or “codon optimization” refers to the process of modifying a nucleic acid sequence for enhanced expression in the cells of the vertebrate of interest, e.g., mouse or human, by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using e.g., Aptagen's GENE FORGE® codon optimization and custom gene synthesis platform (Aptagen, Inc., 2190 Fox Mill Rd. Suite 300, Herndon, Va. 20171) or another publicly available database.

Many organisms display a bias for use of particular codons to code for insertion of a particular amino acid in a growing peptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

Given the large number of gene sequences available for a wide variety of animal, plant and microbial species, it is possible to calculate the relative frequencies of codon usage (Nakamura, Y., et al. “Codon usage tabulated from the international DNA sequence databases: status for the year 2000” Nucl. Acids Res. 28:292 (2000)).

In some embodiments, the cleaved ceDNA (e.g., donor sequences) and guide RNA target a therapeutic gene. In some embodiments, the guide RNA targets an antibody, or antibody fragment, or antigen-binding fragment thereof, e.g., a neutralizing antibody or antibody fragment and the like.

In particular, the cleaved ceDNA (e.g., donor sequence) and gRNA target one or more therapeutic agent(s), including, but not limited to, for example, protein(s), polypeptide(s), peptide(s), enzyme(s), antibodies, antigen binding fragments, as well as variants, and/or active fragments thereof, for use in the treatment, prophylaxis, and/or amelioration of one or more symptoms of a disease, dysfunction, injury, and/or disorder. Exemplary genes for targeting with the guide RNA are described herein in the section entitled “Method of Treatment”.

There are many structural features of cleavable ceDNA vectors that differ from plasmid-based expression vectors. Cleavable ceDNA vectors may possess one or more of the following features: the lack of original (i.e. not inserted) bacterial DNA, the lack of a prokaryotic origin of replication, the presence of ITR sequences that form hairpins, of the eukaryotic origin (i.e., they are produced in eukaryotic cells), and the absence of bacterial-type DNA methylation or indeed any other methylation considered abnormal by a mammalian host. In general, it is preferred for the present vectors not to contain any prokaryotic DNA but it is contemplated that some prokaryotic DNA may be inserted as an exogenous sequence, as a nonlimiting example in a promoter or enhancer region. Another important feature distinguishing ceDNA vectors from plasmid expression vectors is that ceDNA vectors are single-strand linear DNA having closed ends, while plasmids are always double-stranded DNA.

ceDNA produced by the methods provided herein preferably have a linear and continuous structure rather than a non-continuous structure, as determined by restriction enzyme digestion assay. The linear and continuous structure is believed to be more stable from attack by cellular endonucleases, as well as less likely to be recombined and cause mutagenesis. Thus, a cleavable ceDNA vector in the linear and continuous structure is a preferred embodiment. The continuous, linear, single strand intramolecular duplex ceDNA vector can have covalently bound terminal ends, without sequences encoding AAV capsid proteins. These cleavable ceDNA vectors are structurally distinct from plasmids (including ceDNA plasmids described herein), which are circular duplex nucleic acid molecules of bacterial origin. The complimentary strands of plasmids may be separated following denaturation to produce two nucleic acid molecules, whereas in contrast, cleavable ceDNA vectors, while having complimentary strands, are a single DNA molecule and therefore even if denatured, remain a single molecule. In some embodiments, cleavable ceDNA vectors as described herein can be produced without DNA base methylation of prokaryotic type, unlike plasmids. Therefore, the cleavable ceDNA vectors and ceDNA-plasmids are different both in term of structure (in particular, linear versus circular) and also in view of the methods used for producing and purifying these different objects (see below), and also in view of their DNA methylation which is of prokaryotic type for ceDNA-plasmids and of eukaryotic type for the cleavable ceDNA vector.

There are several advantages of using a cleaved ceDNA as described herein as a donor sequence for gene editing over plasmid-based expression vectors, such advantages include, but are not limited to: 1) plasmids contain bacterial DNA sequences and are subjected to prokaryotic-specific methylation, e.g., 6-methyl adenosine and 5-methyl cytosine methylation, whereas capsid-free AAV vector sequences are of eukaryotic origin and do not undergo prokaryotic-specific methylation; as a result, capsid-free AAV vectors are less likely to induce inflammatory and immune responses compared to plasmids; 2) while plasmids require the presence of a resistance gene during the production process, ceDNA vectors do not; 3) while a circular plasmid is not delivered to the nucleus upon introduction into a cell and requires overloading to bypass degradation by cellular nucleases, ceDNA vectors contain viral cis-elements, i.e., ITRs, that confer resistance to nucleases and can be designed to be targeted and delivered to the nucleus. It is hypothesized that the minimal defining elements indispensable for ITR function are a Rep-binding site (RBS; 5′-GCGCGCTCGCTCGCTC-3′ (SEQ ID NO: 9) for AAV2) and a terminal resolution site (TRS; 5′-AGTTGG-3′ for AAV2) plus a variable palindromic sequence allowing for hairpin formation; and 4) ceDNA vectors do not have the over-representation of CpG dinucleotides often found in prokaryote-derived plasmids that reportedly binds a member of the Toll-like family of receptors, eliciting a T cell-mediated immune response. In contrast, transductions with capsid-free AAV vectors disclosed herein can efficiently target cell and tissue-types that are difficult to transduce with conventional AAV virions using various delivery reagent.

### III. Knock-In of a Desired Nucleic Acid Sequence

The cleavable ceDNA vectors, methods and compositions described herein can be used to introduce a new nucleic acid sequence, correct a mutation of a genomic sequence or introduce a mutation into a target gene sequence in a host cell. Such methods can be referred to as “DNA knock-in systems.” The DNA knock-in system, as described herein, allows donor sequences to be inserted at any desired target site with high efficiency, making it feasible for many uses such as creation of transgenic animals expressing exogenous genes, preparing cell culture models of disease, preparing screening assay systems, modifying gene expression of engineered tissue constructs, modifying (e.g., mutating) a genomic locus, and gene editing, for example by adding an exogenous non-coding sequence (such as sequence tags or regulatory elements) into the genome. The cells and animals produced using methods provided herein can find various applications, for example as cellular therapeutics, as disease models, as research tools, and as humanized animals useful for various purposes.

The DNA knock-in systems of the present disclosure also allow for gene editing techniques using large donor sequences (<5 kb) to be inserted at any desired target site in a genome, thus providing gene editing of larger genes than current techniques. In some embodiments, large homology arms, for example 50 base pairs to two thousand base pairs, are included providing gene editing with excellent efficiency (higher on-target) and excellent specificity (lower off-target), and in some embodiments, HDR without the use of nucleases.

The DNA knock-in systems of the present disclosure also provide several advantages with respect to the administration of donor sequences for gene editing. First, administering ceDNA vectors as described herein within delivery particles of the present disclosure is not precluded by baseline immunity and therefore can be administered to any and potentially all patients with a particular disorder. Second, administering particles of the present disclosure does not create an adaptive immune response to the delivered therapeutic like that typically raised against viral vector-based delivery systems and therefore embodiments can be re-dosed as needed for clinical effect. Administration of one or more cleavable ceDNA vectors in accordance with the present disclosure, such as in vivo delivery, is repeatable and robust.

In certain embodiments, gene editing with cleavable ceDNA vectors of the present disclosure can be monitored with appropriate biomarkers from treated patients to assess the efficiency of the gene correction, and repeat administrations of the therapeutic product can be made until the appropriate level of gene editing has been achieved.

In another aspect, there is provided a method of generating a genetically modified animal by using the gene knock-in system described herein with cleavable ceDNA vectors in accordance with the present disclosure. These methods are described further below.

In certain embodiments, the present disclosure relates to methods of using a cleavable ceDNA vector for inserting a donor sequence at a predetermined insertion site on a chromosome of a host cell, such as a eukaryotic or prokaryotic cell.

### IV. Gene Editing System Components—General

The components required for gene editing in a cell may include a cleavable ceDNA as described herein, at least one guide RNA (gRNA); and at least one Cas9 enzyme. According to some embodiments, the Cas9 enzyme is a wild-type Cas9 protein or a nicking Cas9 protein (nCas9). According to some embodiments, the nCas9 contains a mutation in the HNH or RuVc domain of Cas. According to some embodiments, the Cas9 enzyme is a S. pyogenes Cas9.

According to some embodiments, the at least one gRNA is capable of binding to both the first gRNA target sequence (TS) and the second gRNA TS.

According to some embodiments, the gene editing system further comprises a second gRNA, wherein the second gRNA is capable of binding to the second gRNA TS, and wherein the at least one gRNA is capable of binding to the first gRNA TS.

Such embodiments increase the efficiency of gene editing compared to approaches that require distinct or various particles to deliver the gene editing components.

In another aspect, the present disclosure relates to kits including one or more cleavable ceDNA vectors for use in any one of the methods described herein.

The methods and compositions described herein also provide for gene editing systems comprising a cellular switch, for example, as described by Oakes et al. Nat. Biotechnol. 34:646-651 (2016), the contents of which are herein incorporated by reference in their entirety.

It is also specifically contemplated herein that the methods and compositions described herein can be performed in a high-throughput manner using methods known in the art (see e.g., Shalem et al. Nat Rev Genet 16:299-311 (2015); Shalem et al. Science 343:84-88 (2014); the contents of each of which are incorporated herein by reference in their entirety.

### V. ITRs

As disclosed herein, cleavable ceDNA vectors contain, e.g., a transgene cassette positioned between two inverted terminal repeat (ITR) sequences, where the ITR sequences can be an asymmetrical ITR pair or a symmetrical- or substantially symmetrical ITR pair, as these terms are defined herein. A cleavable ceDNA disclosed herein can comprise ITR sequences that are selected from any of: (i) at least one WT ITR and at least one modified AAV inverted terminal repeat (mod-ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization, where the methods of the present disclosure may further include a delivery system, such as but not limited to a liposome nanoparticle delivery system.

### A. Symmetrical ITR Pain

In some embodiments, the ITR sequence can be from viruses of the Parvoviridae family, which includes two subfamilies: Parvovirinae, which infect vertebrates, and Densovirinae, which infect insects. The subfamily Parvovirinae (referred to as the parvoviruses) includes the genus Dependovirus, the members of which, under most conditions, require coinfection with a helper virus such as adenovirus or herpes virus for productive infection. The genus Dependovirus includes adeno-associated virus (AAV), which normally infects humans (e.g., serotypes 2, 3A, 3B. 5, and 6) or primates (e.g., serotypes 1 and 4), and related viruses that infect other warm-blooded animals (e.g., bovine, canine, equine, and ovine adeno-associated viruses). The parvoviruses and other members of the Parvoviridae family are generally described in Kenneth I. Berns, “Parvoviridae: The Viruses and Their Replication,” Chapter 69 in FIELDS VIROLOGY (3d Ed. 1996).

While ITRs exemplified in the specification and Examples herein are AAV2 WT-ITRs, one of ordinary skill in the art is aware that one can as stated above use ITRs from any known parvovirus, for example a dependovirus such as AAV (e.g., AAV1, AAV2, AAV3, AAV4, AAV5, AAV 5, AAV7, AAV8, AAV9, AAV10, AAV 11, AAV12, AAVrh8, AAVrh10, AAV-DJ, and AAV-DJ8 genome. E.g., NCBI: NC 002077; NC 001401; NC001729; NC001829; NC006152; NC 006260; NC 006261), chimeric ITRs, or ITRs from any synthetic AAV. In some embodiments, the AAV can infect warm-blooded animals, e.g., avian (AAAV), bovine (BAAV), canine, equine, and ovine adeno-associated viruses. In some embodiments the ITR is from B19 parvovirus (GenBank Accession No: NC 000883), Minute Virus from Mouse (MVM) (GenBank Accession No. NC 001510); goose parvovirus (GenBank Accession No. NC 001701); snake parvovirus 1 (GenBank Accession No. NC 006148). In some embodiments, the 5′ WT-ITR can be from one serotype and the 3′ WT-ITR from a different serotype, as discussed herein.

An ordinarily skilled artisan is aware that ITR sequences have a common structure of a double-stranded Holliday junction, which typically is a T-shaped or Y-shaped hairpin structure, where each WT-ITR is formed by two palindromic arms or loops (B-B′ and C-C′) embedded in a larger palindromic arm (A-A′), and a single stranded D sequence, (where the order of these palindromic sequences defines the flip or flop orientation of the ITR). See, for example, structural analysis and sequence comparison of ITRs from different AAV serotypes (AAV1-AAV6) and described in Grimm et al., J. Virology, 2006; 80(1); 426-439: Yan et al., J. Virology, 2005; 364-379; Duan et al., Virology 1999; 261: 8-14. One of ordinary skill in the art can readily determine WT-ITR sequences from any AAV serotype for use in a cleavable ceDNA vector or ceDNA-plasmid based on the exemplary AAV2 ITR sequences provided herein. See, for example, the sequence comparison of ITRs from different AAV serotypes (AAV1-AAV6, and avian AAV (AAAV) and bovine AAV (BAAV)) described in Grimm et al., J. Virology, 2006; 80(1); 426-439; that show the % identity of the left ITR of AAV2 to the left ITR from other serotypes: AAV-1 (84%), AAV-3 (86%), AAV-4 (79%), AAV-5 (58%), AAV-6 (left ITR) (100%) and AAV-6 (right ITR) (82%).

As discussed herein, in some embodiments a cleavable ceDNA can comprise symmetric ITR sequences (e.g., a symmetrical ITR pair), where the 5′ ITR and the 3′ ITR can have the same symmetrical three-dimensional organization with respect to each other. (i.e., symmetrical or substantially symmetrical). That is a cleavable ceDNA comprises ITR sequences that have a symmetrical three-dimensional spatial organization such that their structure is the same shape in geometrical space, or have the same A, C-C′ and B-B′ loops in 3D space (i.e., they are the same or are mirror images with respect to each other). In such an embodiment, a symmetrical ITR pair, or substantially symmetrical ITR pair can be modified ITRs (e.g., mod-ITRs) that are not wild-type ITRs. A mod-ITR pair can have the same sequence which has one or more modifications from wild-type ITR and are reverse complements (inverted) of each other. In alternative embodiments, a modified ITR pair are substantially symmetrical as defined herein, that is, the modified ITR pair can have a different sequence but have corresponding or the same symmetrical three-dimensional shape.

### (i) Wildtype ITRs

In some embodiments, the symmetrical ITRs, or substantially symmetrical ITRs are wild type (WT-ITRs) as described herein. That is, both ITRs have a wild-type sequence, but do not necessarily have to be WT-ITRs from the same AAV serotype. That is, in some embodiments, one WT-ITR can be from one AAV serotype, and the other WT-ITR can be from a different AAV serotype. In such an embodiment, a WT-ITR pair are substantially symmetrical as defined herein, that is, they can have one or more conservative nucleotide modification while still retaining the symmetrical three-dimensional spatial organization.

Accordingly, as disclosed herein, cleavable ceDNAs comprise a transgene cassette positioned between two flanking wild-type inverted terminal repeat (WT-ITR) sequences, that are either the reverse complement (inverted) of each other, or alternatively, are substantially symmetrical relative to each other—that is a WT-ITR pair have symmetrical three-dimensional spatial organization. In some embodiments, a wild-type ITR sequence (e.g., AAV WT-ITR) comprises a functional Rep binding site (RBS; e.g., 5′-GCCYCGCTCGCTCGCTC-3′ for AAV2, SEQ ID NO: 1) and a functional terminal resolution site (TRS; e.g., 5′-AGTT-3′).

In one aspect, ceDNA vectors are obtainable from a vector polynucleotide that encodes a heterologous nucleic acid operatively positioned between two WT inverted terminal repeat sequences (WT-ITRs) (e.g., AAV WT-ITRs). That is, both ITRs have a wild-type sequence, but do not necessarily have to be WT-ITRs from the same AAV serotype. That is, in some embodiments, one WT-ITR can be from one AAV serotype, and the other WT-ITR can be from a different AAV serotype. In such an embodiment, the WT-ITR pair are substantially symmetrical as defined herein, that is, they can have one or more conservative nucleotide modification while still retaining the symmetrical three-dimensional spatial organization. In some embodiments, the 5′ WT-ITR is from one AAV serotype, and the 3′ WT-ITR is from the same or a different AAV serotype. In some embodiments, the 5′ WT-ITR and the 3′WT-ITR are mirror images of each other, that is they are symmetrical. In some embodiments, the 5′ WT-ITR and the 3′ WT-ITR are from the same AAV serotype.

WT ITRs are well known. In one embodiment the two ITRs are from the same AAV2 serotype. In certain embodiments one can use WT from other serotypes. There are a number of serotypes that are homologous, e.g., AAV2, AAV4, AAV6, AAV8. In one embodiment, closely homologous ITRs (e.g., ITRs with a similar loop structure) can be used. In another embodiment, one can use AAV WT ITRs that are more diverse, e.g., AAV2 and AAV5, and still another embodiment, one can use an ITR that is substantially WT—that is, it has the basic loop structure of the WT but some conservative nucleotide changes that do not alter or affect the properties. When using WT-ITRs from the same viral serotype, one or more regulatory sequences may further be used. In certain embodiments, the regulatory sequence is a regulatory switch that permits modulation of the activity of the cleavable ceDNA.

In some embodiments, one aspect of the technology described herein relates to a cleavable non-viral capsid-free DNA vector with covalently-closed ends (ceDNA vector), wherein the cleavable ceDNA vector comprises a first inverted terminal repeat (ITR), a first guide RNA (gRNA) target sequence (TS), a first protospacer adjacent motif (PAM), at least one transgene cassette, a second protospacer adjacent motif (PAM), a second gRNA target sequence (TS), and a second ITR), wherein the WT-ITRs can be from the same serotype, different serotypes or substantially symmetrical with respect to each other (i.e., have the symmetrical three-dimensional spatial organization such that their structure is the same shape in geometrical space, or have the same A. C-C′ and B-B′ loops in 3D space). In some embodiments, the symmetric WT-ITRs comprises a functional terminal resolution site and a Rep binding site. In some embodiments, the heterologous nucleic acid sequence encodes a transgene, and wherein the vector is not in a viral capsid.

In some embodiments, the WT-ITRs are the same but the reverse complement of each other. For example, the sequence AACG in the 5′ ITR may be CGTT (i.e., the reverse complement) in the 3′ ITR at the corresponding site. In one example, the 5′ WT-ITR sense strand comprises the sequence of ATCGATCG and the corresponding 3′ WT-ITR sense strand comprises CGATCGAT (i.e., the reverse complement of ATCGATCG). In some embodiments, the WT-ITRs ceDNA further comprises a terminal resolution site and a replication protein binding site (RPS) (sometimes referred to as a replicative protein binding site), e.g., a Rep binding site.

Exemplary WT-ITR sequences for use in the cleavable ceDNA vectors comprising WT-ITRs are shown in Table 2 herein, which shows pairs of WT-ITRs (5′ WT-ITR and the 3′ WT-ITR).

As an exemplary example, the present disclosure provides a cleavable closed-ended DNA vector comprising a promoter operably linked to a transgene, with or without the regulatory switch, where the cleavable ceDNA is devoid of capsid proteins and is: (a) produced from a cleavable ceDNA-plasmid-that encodes WT-ITRs, where each WT-ITR has the same number of intramolecularly duplexed base pairs in its hairpin secondary configuration (preferably excluding deletion of any AAA or TTT terminal loop in this configuration compared to these reference sequences), and (b) is identified as ceDNA using the assay for the identification of ceDNA by agarose gel electrophoresis under native gel and denaturing conditions.

In some embodiments, the flanking WT-ITRs are substantially symmetrical to each other. In this embodiment the 5′ WT-ITR can be from one serotype of AAV, and the 3′ WT-ITR from a different serotype of AAV, such that the WT-ITRs are not identical reverse complements. For example, the 5′ WT-ITR can be from AAV2, and the 3′ WT-ITR from a different serotype (e.g., AAV1, 3, 4, 5, 6, 7, 8, 9, 10, 11, and 12. In some embodiments, WT-ITRs can be selected from two different parvoviruses selected from any to of: AAV1, AAV2, AAV3, AAV4, AAV5, AAV6, AAV7, AAV8, AAV9, AAV10, AAV11, AAV12, AAV13, snake parvovirus (e.g., royal python parvovirus), bovine parvovirus, goat parvovirus, avian parvovirus, canine parvovirus, equine parvovirus, shrimp parvovirus, porcine parvovirus, or insect AAV. In some embodiments, such a combination of WT ITRs is the combination of WT-ITRs from AAV2 and AAV6. In one embodiment, the substantially symmetrical WT-ITRs are when one is inverted relative to the other ITR at least 90% identical, at least 95% identical, at least 96% . . . 97% . . . 98% . . . 99% . . . 99.5% and all points in between, and has the same symmetrical three-dimensional spatial organization. In some embodiments, a WT-ITR pair are substantially symmetrical as they have symmetrical three-dimensional spatial organization, e.g., have the same 3D organization of the A, C-C′, B-B′ and D arms. In one embodiment, a substantially symmetrical WT-ITR pair are inverted relative to the other, and are at least 95% identical, at least 96% . . . 97% . . . 98% . . . 99% . . . 99.5% and all points in between, to each other, and one WT-ITR retains the Rep-binding site (RBS) of 5′-GCGCGCTCGCTCGCTC-3′ (SEQ ID NO: 1) and a terminal resolution site (trs). In some embodiments, a substantially symmetrical WT-ITR pair are inverted relative to each other, and are at least 95% identical, at least 96% . . . 97% . . . 98% . . . 99% . . . 99.5% and all points in between, to each other, and one WT-ITR retains the Rep-binding site (RBS) of 5-GCGCGCTCGCTCGCTC-3′ (SEQ ID NO: 1) and a terminal resolution site (trs) and in addition to a variable palindromic sequence allowing for hairpin secondary structure formation. Homology can be determined by standard means well known in the art such as BLAST (Basic Local Alignment Search Tool), BLASTN at default setting.

In some embodiments, the structural element of the ITR can be any structural element that is involved in the functional interaction of the ITR with a large Rep protein (e.g., Rep 78 or Rep 68). In certain embodiments, the structural element provides selectivity to the interaction of an ITR with a large Rep protein, i.e., determines at least in part which Rep protein functionally interacts with the ITR. In other embodiments, the structural element physically interacts with a large Rep protein when the Rep protein is bound to the ITR. Each structural element can be, e.g., a secondary structure of the ITR, a nucleotide sequence of the ITR, a spacing between two or more elements, or a combination of any of the above. In one embodiment, the structural elements are selected from the group consisting of an A and an A′ arm, a B and a B′ arm, a C and a C′ arm, a D arm, a Rep binding site (RBE) and an RBE′ (i.e., complementary RBE sequence), and a terminal resolution sire (trs).

By way of example only, Table 1 indicates exemplary combinations of WT-ITRs.

Table 1: Exemplary combinations of WT-ITRs from the same serotype or different serotypes, or different paroviruses. The order shown is not indicative of the ITR position, for example, “AAV1, AAV2” demonstrates that the cleavable ceDNA can comprise a WT-AAV1 ITR in the 5′ position, and a WT-AAV2 ITR in the 3′ position, or vice versa, a WT-AAV2 ITR the 5′ position, and a WT-AAV1 ITR in the 3′ position. Abbreviations: AAV serotype 1 (AAV1), AAV serotype 2 (AAV2), AAV serotype 3 (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), or AAV serotype 12 (AAV12); AAVrh8, AAVrh10, AAV-DJ, and AAV-DJ8 genome (E.g., NCBI: NC 002077; NC 001401; NC001729; NC001829; NC006152; NC 006260; NC 006261), ITRs from warm-blooded animals (avian AAV (AAAV), bovine AAV (BAAV), canine, equine, and ovine AAV), ITRs from B19 parvovirus (GenBank Accession No: NC 000883), Minute Virus from Mouse (MVM) (GenBank Accession No. NC 001510); Goose: goose parvovirus (GenBank Accession No. NC 001701); snake: snake parvovirus 1 (GenBank Accession No. NC 006148).

TABLE 1

| | | | | |
|---|---|---|---|---|
| AAV1, AAV1 | AAV2, AAV2 | AAV3, AAV3 | AAV4, AAV4 | AAV5, AAV5 |
| AAV1, AAV2 | AAV2, AAV3 | AAV3, AAV4 | AAV4, AAV5 | AAV5, AAV6 |
| AAV1, AAV3 | AAV2, AAV4 | AAV3, AAV5 | AAV4, AAV6 | AAV5, AAV7 |
| AAV1, AAV4 | AAV2, AAV5 | AAV3, AAV6 | AAV4, AAV7 | AAV5, AAV8 |
| AAV1, AAV5 | AAV2, AAV6 | AAV3, AAV7 | AAV4, AAV8 | AAV5, AAV9 |
| AAV1, AAV6 | AAV2, AAV7 | AAV3, AAV8 | AAV4, AAV9 | AAV5, AAV10 |
| AAV1, AAV7 | AAV2, AAV8 | AAV3, AAV9 | AAV4, AAV10 | AAV5, AAV11 |
| AAV1, AAV8 | AAV2, AAV9 | AAV3, AAV10 | AAV4, AAV11 | AAV5, AAV12 |
| AAV1, AAV9 | AAV2, AAV10 | AAV3, AAV11 | AAV4, AAV12 | AAV5, AAVRH8 |
| AAV1, AAV10 | AAV2, AAV11 | AAV3, AAV12 | AAV4, AAVRH8 | AAV5, AAVRH10 |
| AAV1, AAV11 | AAV2, AAV12 | AAV3, AAVRH8 | AAV4, AAVRH10 | AAV5, AAV13 |
| AAV1, AAV12 | AAV2, AAVRH8 | AAV3, AAVRH10 | AAV4, AAV13 | AAV5, AAVDJ |
| AAV1, AAVRH8 | AAV2, AAVRH10 | AAV3, AAV13 | AAV4, AAVDJ | AAV5, AAVDJ8 |
| AAV1, AAVRH10 | AAV2, AAV13 | AAV3, AAVDJ | AAV4, AAVDJ8 | AAV5, AVIAN |
| AAV1, AAV13 | AAV2, AAVDJ | AAV3, AAVDJ8 | AAV4, AVIAN | AAV5, BOVINE |
| AAV1, AAVDJ | AAV2, AAVDJ8 | AAV3, AVIAN | AAV4, BOVINE | AAV5, CANINE |
| AAV1, AAVDJ8 | AAV2, AVIAN | AAV3, BOVINE | AAV4, CANINE | AAV5, EQUINE |
| AAV1, AVIAN | AAV2, BOVINE | AAV3, CANINE | AAV4, EQUINE | AAV5, GOAT |
| AAV1, BOVINE | AAV2, CANINE | AAV3, EQUINE | AAV4, GOAT | AAV5, SHRIMP |
| AAV1, CANINE | AAV2, EQUINE | AAV3, GOAT | AAV4, SHRIMP | AAV5, PORCINE |
| AAV1, EQUINE | AAV2, GOAT | AAV3, SHRIMP | AAV4, PORCINE | AAV5, INSECT |
| AAV1, GOAT | AAV2, SHRIMP | AAV3, PORCINE | AAV4, INSECT | AAV5, OVINE |
| AAV1, SHRIMP | AAV2, PORCINE | AAV3, INSECT | AAV4, OVINE | AAV5, B19 |
| AAV1, PORCINE | AAV2, INSECT | AAV3, OVINE | AAV4, B19 | AAV5, MVM |
| AAV1, INSECT | AAV2, OVINE | AAV3, B19 | AAV4, MVM | AAV5, GOOSE |
| AAV1, OVINE | AAV2, B19 | AAV3, MVM | AAV4, GOOSE | AAV5, SNAKE |
| AAV1, B19 | AAV2, MVM | AAV3, GOOSE | AAV4, SNAKE | |
| AAV1, MVM | AAV2, GOOSE | AAV3, SNAKE | | |
| AAV1, GOOSE | AAV2, SNAKE | | | |
| AAV1, SNAKE | | | | |
| AAV6, AAV6 | AAV7, AAV7 | AAV8, AAV8 | AAV9, AAV9 | AAV10, AAV10 |
| AAV6, AAV7 | AAV7, AAV8 | AAV8, AAV9 | AAV9, AAV10 | AAV10, AAV11 |
| AAV6, AAV8 | AAV7, AAV9 | AAV8, AAV10 | AAV9, AAV11 | AAV10, AAV12 |
| AAV6, AAV9 | AAV7, AAV10 | AAV8, AAV11 | AAV9, AAV12 | AAV10, AAVRH8 |
| AAV6, AAV10 | AAV7, AAV11 | AAV8, AAV12 | AAV9, AAVRH8 | AAV10, AAVRH10 |
| AAV6, AAV11 | AAV7, AAV12 | AAV8, AAVRH8 | AAV9, AAVRH10 | AAV10, AAV13 |
| AAV6, AAV12 | AAV7, AAVRH8 | AAV8, AAVRH10 | AAV9, AAV13 | AAV10, AAVDJ |
| AAV6, AAVRH8 | AAV7, AAVRH10 | AAV8, AAV13 | AAV9, AAVDJ | AAV10, AAVDJ8 |
| AAV6, AAVRH10 | AAV7, AAV13 | AAV8, AAVDJ | AAV9, AAVDJ8 | AAV10, AVIAN |
| AAV6, AAV13 | AAV7, AAVDJ | AAV8, AAVDJ8 | AAV9, AVIAN | AAV10, BOVINE |
| AAV6, AAVDJ | AAV7, AAVDJ8 | AAV8, AVIAN | AAV9, BOVINE | AAV10, CANINE |
| AAV6, AAVDJ8 | AAV7, AVIAN | AAV8, BOVINE | AAV9, CANINE | AAV10, EQUINE |
| AAV6, AVIAN | AAV7, BOVINE | AAV8, CANINE | AAV9, EQUINE | AAV10, GOAT |
| AAV6, BOVINE | AAV7, CANINE | AAV8, EQUINE | AAV9, GOAT | AAV10, SHRIMP |
| AAV6, CANINE | AAV7, EQUINE | AAV8, GOAT | AAV9, SHRIMP | AAV10, PORCINE |
| AAV6, EQUINE | AAV7, GOAT | AAV8, SHRIMP | AAV9, PORCINE | AAV10, INSECT |
| AAV6, GOAT | AAV7, SHRIMP | AAV8, PORCINE | AAV9, INSECT | AAV10, OVINE |
| AAV6, SHRIMP | AAV7, PORCINE | AAV8, INSECT | AAV9, OVINE | AAV10, B19 |
| AAV6, PORCINE | AAV7, INSECT | AAV8, OVINE | AAV9, B19 | AAV10, MVM |
| AAV6, INSECT | AAV7, OVINE | AAV8, B19 | AAV9, MVM | AAV10, GOOSE |
| AAV6, OVINE | AAV7, B19 | AAV8, MVM | AAV9, GOOSE | AAV10, SNAKE |
| AAV6, B19 | AAV7, MVM | AAV8, GOOSE | AAV9, SNAKE | |
| AAV6, MVM | AAV7, GOOSE | AAV8, SNAKE | | |
| AAV6, GOOSE | AAV7, SNAKE | | | |
| AAV6, SNAKE | | | | |
| AAV11, AAV11 | AAV12, AAV12 | AAVRH8, AAVRH8 | AAVRH10, AAVRH10 | AAV13, AAV13 |
| AAV11, AAV12 | AAV12, AAVRH8 | AAVRH8, AAVRH10 | AAVRH10, AAV13 | AAV13, AAVDJ |
| AAV11, AAVRH8 | AAV12, AAVRH10 | AAVRH8, AAV13 | AAVRH10, AAVDJ | AAV13, AAVDJ8 |
| AAV11, AAVRH10 | AAV12, AAV13 | AAVRH8, AAVDJ | AAVRH10, AAVDJ8 | AAV13, AVIAN |
| AAV11, AAV13 | AAV12, AAVDJ | AAVRH8, AAVDJ8 | AAVRH10, AVIAN | AAV13, BOVINE |
| AAV11, AAVDJ | AAV12, AAVDJ8 | AAVRH8, AVIAN | AAVRH10, BOVINE | AAV13, CANINE |
| AAV11, AAVDJ8 | AAV12, AVIAN | AAVRH8, BOVINE | AAVRH10, CANINE | AAV13, EQUINE |
| AAV11, AVIAN | AAV12, BOVINE | AAVRH8, CANINE | AAVRH10, EQUINE | AAV13, GOAT |
| AAV11, BOVINE | AAV12, CANINE | AAVRH8, EQUINE | AAVRH10, GOAT | AAV13, SHRIMP |
| AAV11, CANINE | AAV12, EQUINE | AAVRH8, GOAT | AAVRH10, SHRIMP | AAV13, PORCINE |
| AAV11, EQUINE | AAV12, GOAT | AAVRH8, SHRIMP | AAVRH10, PORCINE | AAV13, INSECT |
| AAV11, GOAT | AAV12, SHRIMP | AAVRH8, PORCINE | AAVRH10, INSECT | AAV13, OVINE |
| AAV11, SHRIMP | AAV12, PORCINE | AAVRH8, INSECT | AAVRH10, OVINE | AAV13, B19 |
| AAV11, PORCINE | AAV12, INSECT | AAVRH8, OVINE | AAVRH10, B19 | AAV13, MVM |
| AAV11, INSECT | AAV12, OVINE | AAVRH8, B19 | AAVRH10, MVM | AAV13, GOOSE |
| AAV11, OVINE | AAV12, B19 | AAVRH8, MVM | AAVRH10, GOOSE | AAV13, SNAKE |
| AAV11, B19 | AAV12, MVM | AAVRH8, GOOSE | AAVRH10, SNAKE | |
| AAV11, MVM | AAV12, GOOSE | AAVRH8, SNAKE | | |
| AAV11, GOOSE | AAV12, SNAKE | | | |
| AAV11, SNAKE | | | | |
| AAVDJ, AAVDJ | AAVDJ8, AVVDJ8 | AVIAN, AVIAN | BOVINE, BOVINE | CANINE, CANINE |
| AAVDJ, AAVDJ8 | AAVDJ8, AVIAN | AVIAN, BOVINE | BOVINE, CANINE | CANINE, EQUINE |
| AAVDJ, AVIAN | AAVDJ8, BOVINE | AVIAN, CANINE | BOVINE, EQUINE | CANINE, GOAT |
| AAVDJ, BOVINE | AAVDJ8, CANINE | AVIAN, EQUINE | BOVINE, GOAT | CANINE, SHRIMP |
| AAVDJ, CANINE | AAVDJ8, EQUINE | AVIAN, GOAT | BOVINE, SHRIMP | CANINE, PORCINE |
| AAVDJ, EQUINE | AAVDJ8, GOAT | AVIAN, SHRIMP | BOVINE, PORCINE | CANINE, INSECT |
| AAVDJ, GOAT | AAVDJ8, SHRIMP | AVIAN, PORCINE | BOVINE, INSECT | CANINE, OVINE |
| AAVDJ, SHRIMP | AAVDJ8, PORCINE | AVIAN, INSECT | BOVINE, OVINE | CANINE, B19 |
| AAVDJ, PORCINE | AAVDJ8, INSECT | AVIAN, OVINE | BOVINE, B19 | CANINE, MVM |
| AAVDJ, INSECT | AAVDJ8, OVINE | AVIAN, B19 | BOVINE, MVM | CANINE, GOOSE |
| AAVDJ, OVINE | AAVDJ8, B19 | AVIAN, MVM | BOVINE, GOOSE | CANINE, SNAKE |
| AAVDJ, B19 | AAVDJ8, MVM | AVIAN, GOOSE | BOVINE, SNAKE | |
| AAVDJ, MVM | AAVDJ8, GOOSE | AVIAN, SNAKE | | |
| AAVDJ, GOOSE | AAVDJ8, SNAKE | | | |
| AAVDJ, SNAKE | | | | |
| EQUINE, EQUINE | GOAT, GOAT | SHRIMP, SHRIMP | PORCINE, PORCINE | INSECT, INSECT |
| EQUINE, GOAT | GOAT, SHRIMP | SHRIMP, PORCINE | PORCINE, INSECT | INSECT, OVINE |
| EQUINE, SHRIMP | GOAT, PORCINE | SHRIMP, INSECT | PORCINE, OVINE | INSECT, B19 |
| EQUINE, PORCINE | GOAT, INSECT | SHRIMP, OVINE | PORCINE, B19 | INSECT, MVM |
| EQUINE, INSECT | GOAT, OVINE | SHRIMP, B19 | PORCINE, MVM | INSECT, GOOSE |
| EQUINE, OVINE | GOAT, B19 | SHRIMP, MVM | PORCINE, GOOSE | INSECT, SNAKE |
| EQUINE, B19 | GOAT, MVM | SHRIMP, GOOSE | PORCINE, SNAKE | |
| EQUINE, MVM | GOAT, GOOSE | SHRIMP, SNAKE | | |
| EQUINE, GOOSE | GOAT, SNAKE | | | |
| EQUINE, SNAKE | | | | |
| OVINE, OVINE | B19, B19 | MVM, MVM | GOOSE, GOOSE | SNAKE, SNAKE |
| OVINE, B19 | B19, MVM | MVM, GOOSE | GOOSE, SNAKE | |
| OVINE, MVM | B19, GOOSE | MVM, SNAKE | | |
| OVINE, GOOSE | B19, SNAKE | | | |
| OVINE, SNAKE | | | | |

By way of example only, Table 2 shows the sequences of exemplary WT-ITRs from some different AAV serotypes.

TABLE 2

| AAV | | |
|---|---|---|
| serotype | 5′ WT-ITR (LEFT) | 3′ WT-ITR (RIGHT) |
| AAV1 | SEQ ID NO: 98 | SEQ ID NO: 99 |
| | | (from Kay et al., J Virol, 2006, 426-439, |
| | | FIG. 1A) |
| AAV2 | SEQ ID NO: 100 | SEQ ID NO: 101 |
| AAV3 | SEQ ID NO: 102 | SEQ ID NO: 103 |
| | | (from Kay et al., J Virol, 2006, 426-439, |
| | | FIG. 1A) |
| AAV4 | SEQ ID NO: 104 | SEQ ID NO: 105 |
| AAV5 | SEQ ID NO: 106 | SEQ ID NO: 107 |
| AAV6 | SEQ ID NO: 108 | SEQ ID NO: 97 |
| | | (from Kay et al., J Virol, 2006, 426-439, |
| | | FIG. 1A) |

In some embodiments, the nucleotide sequence of the WT-ITR sequence can be modified (e.g., by modifying 1, 2, 3, 4 or 5, or more nucleotides or any range therein), whereby the modification is a substitution for a complementary nucleotide, e.g., G for a C, and vice versa, and T for an A, and vice versa.

In certain embodiments of the present invention, the cleavable ceDNA vector does not have a WT-ITR as shown in Table 2.

The cleavable ceDNA vector described herein can include WT-ITR structures that retains an operable RBE, trs and RBE′ portion. In some embodiments, the cleavable ceDNA vector contains one or more functional WT-ITR polynucleotide sequences that comprise a Rep-binding site (RBS; 5′-GCGCGCTCGCTCGCTC-3′ (SEQ ID NO: 1) for AAV2) and a terminal resolution site (TRS; 5′-AGTT). In some embodiments, at least one WT-ITR is functional. In alternative embodiments, where a cleavable ceDNA vector comprises two WT-ITRs that are substantially symmetrical to each other, at least one WT-ITR is functional and at least one WT-ITR is non-functional.

B. Modified ITRs (mod-ITRs) in general for ceDNA vectors comprising asymmetric ITR pairs or symmetric ITR pairs

As discussed herein, a cleavable ceDNA vector can comprise a symmetrical ITR pair or an asymmetrical ITR pair. In both instances, the ITRs can be modified ITRs—the difference being that in the first instance (i.e., symmetric mod-ITRs), the mod-ITRs have the same three-dimensional spatial organization (i.e., have the same A-A′, C-C′ and B-B′ arm configurations), whereas in the second instance (i.e., asymmetric mod-ITRs), the mod-ITRs have a different three-dimensional spatial organization (i.e., have a different configuration of A-A′, C-C′ and B-B′ arms).

In some embodiments, a modified ITR is an ITRs that is modified by deletion, insertion, and/or substitution as compared to a wild-type ITR sequence (e.g., AAV ITR). In some embodiments, at least one of the ITRs in the cleavable ceDNA vector comprises a functional Rep binding site (RBS; e.g., 5′-GCGCGCTCGCTCGCTC-3′ for AAV2, SEQ ID NO: 1) and a functional terminal resolution site (TRS: e.g., 5′-AGTT-3′) In one embodiment, at least one of the ITRs is a non-functional ITR. In one embodiment, the different or modified ITRs are not each wild type ITRs from different serotypes.

Specific alterations and mutations in the ITRs are described in detail herein, but in the context of ITRs. “altered” or “mutated” or “modified”, it indicates that nucleotides have been inserted, deleted, and/or substituted relative to the wild-type, reference, or original ITR sequence. The altered or mutated ITR can be an engineered ITR. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature.

In some embodiments, a mod-ITR may be synthetic. In one embodiment, a synthetic ITR is based on ITR sequences from more than one AAV serotype. In another embodiment, a synthetic ITR includes no AAV-based sequence. In yet another embodiment, a synthetic ITR preserves the ITR structure described above although having only some or no AAV-sourced sequence. In some aspects, a synthetic ITR may interact preferentially with a wild type Rep or a Rep of a specific serotype, or in some instances will not be recognized by a wild-type Rep and be recognized only by a mutated Rep.

The skilled artisan can determine the corresponding sequence in other serotypes by known means. For example, determining if the change is in the A, A′, B, B′, C, C′ or D region and determine the corresponding region in another serotype. One can use BLAST® (Basic Local Alignment Search Tool) or other homology alignment programs at default status to determine the corresponding sequence. The invention further provides populations and pluralities of cleavable ceDNA vectors comprising mod-ITRs from a combination of different AAV serotypes—that is, one mod-ITR can be from one AAV serotype and the other mod-ITR can be from a different serotype. Without wishing to be bound by theory, in one embodiment one ITR can be from or based on an AAV2 ITR sequence and the other ITR of the cleavable ceDNA vector can be from or be based on any one or more ITR sequence of AAV serotype 1 (AAV1), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV 11), or AAV serotype 12 (AAV12).

Any parvovirus ITR can be used as an ITR or as a base ITR for modification. Preferably, the parvovirus is a dependovirus. More preferably AAV. The serotype chosen can be based upon the tissue tropism of the serotype. AAV2 has a broad tissue tropism, AAV1 preferentially targets to neuronal and skeletal muscle, and AAV5 preferentially targets neuronal, retinal pigmented epithelia, and photoreceptors. AAV6 preferentially targets skeletal muscle and lung. AAV8 preferentially targets liver, skeletal muscle, heart, and pancreatic tissues. AAV9 preferentially targets liver, skeletal and lung tissue. In one embodiment, the modified ITR is based on an AAV2 ITR.

More specifically, the ability of a structural element to functionally interact with a particular large Rep protein can be altered by modifying the structural element. For example, the nucleotide sequence of the structural element can be modified as compared to the wild-type sequence of the ITR. In one embodiment, the structural element (e.g., A arm, A′ arm, B arm, B′ arm, C arm, C′ arm, D arm, RBE, RBE′, and trs) of an ITR can be removed and replaced with a wild-type structural element from a different parvovirus. For example, the replacement structure can be from AAV1, AAV2, AAV3, AAV4, AAV5, AAV6, AAV7, AAV8, AAV9, AAV10, AAV11, AAV12, AAV13, snake parvovirus (e.g., royal python parvovirus), bovine parvovirus, goat parvovirus, avian parvovirus, canine parvovirus, equine parvovirus, shrimp parvovirus, porcine parvovirus, or insect AAV. For example, the ITR can be an AAV2 ITR and the A or A′ arm or RBE can be replaced with a structural element from AAV5. In another example, the ITR can be an AAV5 ITR and the C or C′ arms, the RBE, and the trs can be replaced with a structural element from AAV2. In another example, the AAV ITR can be an AAV5 ITR with the B and B′ arms replaced with the AAV2 ITR B and B′ arms.

By way of example only, Table 3 indicates exemplary modifications of at least one nucleotide (e.g., a deletion, insertion and/or substitution) in regions of a modified ITR, where X is indicative of a modification of at least one nucleic acid (e.g., a deletion, insertion and/or substitution) in that section relative to the corresponding wild-type ITR. In some embodiments, any modification of at least one nucleotide (e.g., a deletion, insertion and/or substitution) in any of the regions of C and/or C′ and/or B and/or B′ retains three sequential T nucleotides (i.e., TTT) in at least one terminal loop. For example, if the modification results in any of: a single arm ITR (e.g., single C-C′ arm, or a single B-B′ arm), or a modified C-B′ arm or C′-B arm, or a two arm ITR with at least one truncated arm (e.g., a truncated C-C′ arm and/or truncated B-B′ arm), at least the single arm, or at least one of the arms of a two arm ITR (where one arm can be truncated) retains three sequential T nucleotides (i.e., TTT) in at least one terminal loop. In some embodiments, a truncated C-C′ arm and/or a truncated B-B′ arm has three sequential T nucleotides (i.e., TTT) in the terminal loop.

TABLE 3
Exemplary combinations of modifications of at least one nucleotide
(e.g., a deletion, insertion and/or substitution) to different
B-B′ and C-C′ regions or arms of ITRs (X indicates
a nucleotide modification, e.g., addition, deletion or substitution
of at least one nucleotide in the region).

| B region | B′ region | C region | C′ region |
|---|---|---|---|
| X | | | |
| | X | | |
| X | X | | |
| | | X | |
| | | | X |
| | | X | X |
| X | | X | |
| X | | | X |
| | X | X | |
| | X | | X |
| X | X | X | |
| X | X | | X |
| X | | X | X |
| | X | X | X |
| X | X | X | X |

In some embodiments, mod-ITR for use in a cleavable ceDNA vector comprising an asymmetric ITR pair, or a symmetric mod-ITR pair as disclosed herein can comprise any one of the combinations of modifications shown in Table 3, and also a modification of at least one nucleotide in any one or more of the regions selected from: between A′ and C, between C and C′, between C′ and B, between B and B′ and between B′ and A. In some embodiments, any modification of at least one nucleotide (e.g., a deletion, insertion and/or substitution) in the C or C′ or B or B′ regions, still preserves the terminal loop of the stem-loop. In some embodiments, any modification of at least one nucleotide (e.g., a deletion, insertion and/or substitution) between C and C′ and/or B and B′ retains three sequential T nucleotides (i.e., TTT) in at least one terminal loop. In alternative embodiments, any modification of at least one nucleotide (e.g., a deletion, insertion and/or substitution) between C and C′ and/or B and B′ retains three sequential A nucleotides (i.e., AAA) in at least one terminal loop In some embodiments, a modified ITR for use herein can comprise any one of the combinations of modifications shown in Table 3, and also a modification of at least one nucleotide (e.g., a deletion, insertion and/or substitution) in any one or more of the regions selected from: A′, A and/or D. For example, in some embodiments, a modified ITR for use herein can comprise any one of the combinations of modifications shown in Table 3, and also a modification of at least one nucleotide (e.g., a deletion, insertion and/or substitution) in the A region. In some embodiments, a modified ITR for use herein can comprise any one of the combinations of modifications shown in Table 3, and also a modification of at least one nucleotide (e.g., a deletion, insertion and/or substitution) in the A′ region. In some embodiments, a modified ITR for use herein can comprise any one of the combinations of modifications shown in Table 3, and also a modification of at least one nucleotide (e.g., a deletion, insertion and/or substitution) in the A and/or A′ region. In some embodiments, a modified ITR for use herein can comprise any one of the combinations of modifications shown in Table 3, and also a modification of at least one nucleotide (e.g., a deletion, insertion and/or substitution) in the D region.

In one embodiment, the nucleotide sequence of the structural element can be modified (e.g., by modifying 1, 2, 3, 4, 5, 6, 7, 8, 9, 10, 11, 12, 13, 14, 15, 16, 17, 18, 19, or 20 or more nucleotides or any range therein) to produce a modified structural element. In one embodiment, the specific modifications to the ITRs are exemplified herein. In some embodiments, an ITR can be modified (e.g., by modifying 1, 2, 3, 4, 5, 6, 7, 8, 9, 10, 11, 12, 13, 14, 15, 16, 17, 18, 19, or 20 or more nucleotides or any range therein). In other embodiments, the ITR can have at least 80%, at least 85%, at least 90%, at least 95%, at least 96%, at least 97%, at least 98%, at least 99%, or more sequence identity with one of the modified ITRs or the RBE-containing section of the A-A′ arm and C-C′ and B-B′ arms or shown in Tables 2-9 (i.e., SEQ ID NO: 110-112, 115-190, 200-468) of PCT/US18/49996, which is incorporated herein in its entirety by reference.

In some embodiments, a modified ITR can for example, comprise removal or deletion of all of a particular arm, e.g., all or part of the A-A′ arm, or all or part of the B-B′ arm or all or part of the C-C′ arm, or alternatively, the removal of 1, 2, 3, 4, 5, 6, 7, 8, 9 or more base pairs forming the stem of the loop so long as the final loop capping the stem (e.g., single arm) is still present. In some embodiments, a modified ITR can comprise the removal of 1, 2, 3, 4, 5, 6, 7, 8, 9 or more base pairs from the B-B′ arm. In some embodiments, a modified ITR can comprise the removal of 1, 2, 3, 4, 5, 6, 7, 8, 9 or more base pairs from the C-C′ arm. In some embodiments, a modified ITR can comprise the removal of 1, 2, 3, 4, 5, 6, 7, 8, 9 or more base pairs from the C-C′ arm and the removal of 1, 2, 3, 4, 5, 6, 7, 8, 9 or more base pairs from the B-B′ arm. Any combination of removal of base pairs is envisioned, for example, 6 base pairs can be removed in the C-C arm and 2 base pairs in the B-B′ arm.

In some embodiments, a modified ITR can have between 1 and 50 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) nucleotide deletions relative to a full-length wild-type ITR sequence. In some embodiments, a modified ITR can have between 1 and 30 nucleotide deletions relative to a full-length WT ITR sequence. In some embodiments, a modified ITR has between 2 and 20 nucleotide deletions relative to a full-length wild-type ITR sequence.

In some embodiments, a modified ITR does not contain any nucleotide deletions in the RBE-containing portion of the A or A′ regions, so as not to interfere with DNA replication (e.g., binding to a RBE by Rep protein, or nicking at a terminal resolution site). In some embodiments, a modified ITR encompassed for use herein has one or more deletions in the B, B′, C, and/or C′ region as described herein.

In some embodiments, the cleavable ceDNA vector comprising a symmetric ITR pair or asymmetric ITR pair comprises a regulatory switch as disclosed herein and at least one modified ITR selected having the nucleotide sequence selected from any of the group consisting of: SEQ ID NO: 550-557.

In another embodiment, the structure of the structural element can be modified. For example, the structural element a change in the height of the stem and/or the number of nucleotides in the loop. For example, the height of the stem can be about 2, 3, 4, 5, 6, 7, 8, or 9 nucleotides or more or any range therein. In one embodiment, the stem height can be about 5 nucleotides to about 9 nucleotides and functionally interacts with Rep. In another embodiment, the stem height can be about 7 nucleotides and functionally interacts with Rep. In another example, the loop can have 3, 4, 5, 6, 7, 8, 9, or 10 nucleotides or more or any range therein.

In another embodiment, the number of GAGY binding sites or GAGY-related binding sites within the RBE or extended RBE can be increased or decreased. In one example, the RBE or extended RBE, can comprise 1, 2, 3, 4, 5, or 6 or more GAGY binding sites or any range therein. Each GAGY binding site can independently be an exact GAGY sequence or a sequence similar to GAGY as long as the sequence is sufficient to bind a Rep protein.

In another embodiment, the spacing between two elements (such as but not limited to the RBE and a hairpin) can be altered (e.g., increased or decreased) to alter functional interaction with a large Rep protein. For example, the spacing can be about 1, 2, 3, 4, 5, 6, 7, 8, 9, 10, 11, 12, 13, 14, 15, 16, 17, 18, 19, 20, or 21 nucleotides or more or any range therein.

The cleavable ceDNA vector described herein can include an ITR structure that is modified with respect to the wild type AAV2 ITR structure disclosed herein, but still retains an operable RBE, trs and RBE′ portion. In some embodiments, the cleavable ceDNA vector contains one or more functional ITR polynucleotide sequences that comprise a Rep-binding site (RBS; 5′-GCGCGCTCGCTCGCTC-3′ (SEQ ID NO: 1) for AAV2) and a terminal resolution site (TRS; 5′-AGTT). In some embodiments, at least one ITR (wt or modified ITR) is functional. In alternative embodiments, where a cleavable ceDNA vector comprises two modified ITRs that are different or asymmetrical to each other, at least one modified ITR is functional and at least one modified ITR is non-functional.

In some embodiments, the modified ITR (e.g., the left or right ITR) of the cleavable ceDNA vector described herein has modifications within the loop arm, the truncated arm, or the ceDNA spacer. Exemplary sequences of ITRs having modifications within the loop arm, the truncated arm, or the spacer are listed in Table 2; Table 3: Table 4; Table 5; Table 6; and Tables 7-9 or Table 10A or 10B of PCT application PCT/US18/49996, which is incorporated herein in its entirety by reference. In some embodiments, the modified ITR for use in a cleavable ceDNA vector comprising an asymmetric ITR pair, or symmetric mod-ITR pair is selected from any or a combination of those shown in Tables 2, 3, 4, 5, 6, 7, 8, 9 and 10A-10B of PCT application PCT/US18/49996 which is incorporated herein in its entirety by reference.

Additional exemplary modified ITRs for use in a cleavable ceDNA vector comprising an asymmetric ITR pair, or symmetric mod-ITR pair in each of the above classes are provided in Tables 4A and 4B.

Table 4A and Table 4B show exemplary right and left modified ITRs.

TABLE 4A
Exemplary modified right ITRs.

| ITR Construct | Sequence |
|---|---|
| ITR-18 Right | SEQ ID NO: 110 |
| ITR-19 Right | SEQ ID NO: 111 |
| ITR-20 Right | SEQ ID NO: 112 |
| ITR-21 Right | SEQ ID NO: 113 |
| ITR-22 Right | SEQ ID NO: 114 |
| ITR-23 Right | SEQ ID NO: 115 |
| ITR-24 Right | SEQ ID NO: 116 |
| ITR-25 Right | SEQ ID NO: 117 |
| ITR-26 Right | SEQ ID NO: 118 |
| ITR-27 Right | SEQ ID NO: 119 |
| ITR-28 Right | SEQ ID NO: 120 |
| ITR-29 Right | SEQ ID NO: 121 |
| ITR-30 Right | SEQ ID NO: 122 |
| ITR-31 Right | SEQ ID NO: 123 |
| ITR-32 Right | SEQ ID NO: 124 |
| ITR-49 Right | SEQ ID NO: 125 |
| ITR-50 right | SEQ ID NO: 126 |

TABLE 4B
Exemplary modified left ITRs.

| | |
|---|---|
| ITR-33 Left | SEQ ID NO: 127 |
| ITR-34 Left | SEQ ID NO: 128 |
| ITR-35 Left | SEQ ID NO: 129 |
| ITR-36 Left | SEQ ID NO: 130 |
| ITR-37 Left | SEQ ID NO: 131 |
| ITR-38 Left | SEQ ID NO: 132 |
| ITR-39 Left | SEQ ID NO: 133 |
| ITR-40 Left | SEQ ID NO: 134 |
| ITR-41 Left | SEQ ID NO: 135 |
| ITR-42 Left | SEQ ID NO: 136 |
| ITR-43 Left | SEQ ID NO: 137 |
| ITR-44 Left | SEQ ID NO: 138 |
| ITR-45 Left | SEQ ID NO: 139 |
| ITR-46 Left | SEQ ID NO: 140 |
| ITR-47 Left | SEQ ID NO: 141 |
| ITR-48 Left | SEQ ID NO: 142 |

In one embodiment, a cleavable ceDNA vector comprises two symmetrical mod-ITRs—that is, both ITRs have the same sequence, but are reverse complements (inverted) of each other. In some embodiments, a symmetrical mod-ITR pair comprises at least one or any combination of a deletion, insertion, or substitution relative to wild type ITR sequence from the same AAV serotype. The additions, deletions, or substitutions in the symmetrical ITR are the same but the reverse complement of each other. For example, an insertion of 3 nucleotides in the C region of the 5′ ITR would be reflected in the insertion of 3 reverse complement nucleotides in the corresponding section in the C′ region of the 3′ ITR. Solely for illustration purposes only, if the addition is AACG in the 5′ ITR, the addition is CGTT in the 3′ ITR at the corresponding site. For example, if the 5′ ITR sense strand is ATCGATCG with an addition of AACG between the G and A to result in the sequence ATCGAACGATCG (SEQ ID NO: 143). The corresponding 3′ ITR sense strand is CGATCGAT (the reverse complement of ATCGATCG) with an addition of CGTT (i.e. the reverse complement of AACG) between the T and C to result in the sequence CGATCGTTCGAT (SEQ ID NO: 144) (the reverse complement of ATCGAACGATCG (SEQ ID NO: 143)).

In alternative embodiments, the modified ITR pair are substantially symmetrical as defined herein—that is, the modified ITR pair can have a different sequence but have corresponding or the same symmetrical three-dimensional shape. For example, one modified ITR can be from one serotype and the other modified ITR be from a different serotype, but they have the same mutation (e.g., nucleotide insertion, deletion or substitution) in the same region. Stated differently, for illustrative purposes only, a 5′ mod-ITR can be from AAV2 and have a deletion in the C region, and the 3′ mod-ITR can be from AAV5 and have the corresponding deletion in the C′ region, and provided the 5′ mod-ITR and the 3′ mod-ITR have the same or symmetrical three-dimensional spatial organization, they are encompassed for use herein as a modified ITR pair.

In some embodiments, a substantially symmetrical mod-ITR pair has the same A, C-C′ and B-B′ loops in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A and B-B′ loops in the same shape in geometric space of its cognate mod-ITR. By way of example only, substantially symmetrical ITRs can have a symmetrical spatial organization such that their structure is the same shape in geometrical space. This can occur, e.g., when a G-C pair is modified, for example, to a C-G pair or vice versa, or A-T pair is modified to a T-A pair, or vice versa. Therefore, using the exemplary example above of modified 5′ ITR as a ATCGAACGATCG (SEQ ID NO: 143), and modified 3′ ITR as CGATCGTTCGAT (SEQ ID NO: 144) (i.e., the reverse complement of ATCGAACGATCG (SEQ ID NO: 143)), these modified ITRs would still be symmetrical if, for example, the 5′ ITR had the sequence of ATCGAACCATCG (SEQ ID NO: 145), where G in the addition is modified to C, and the substantially symmetrical 3′ ITR has the sequence of CGATCGTTCGAT (SEQ ID NO: 144), without the corresponding modification of the T in the addition to a. In some embodiments, such a modified ITR pair are substantially symmetrical as the modified ITR pair has symmetrical stereochemistry.

Table 5 shows exemplary symmetric modified ITR pairs (i.e. a left modified ITRs and the symmetric right modified ITR).

TABLE 5
exemplary symmetric modified ITR pairs

| LEFT modified ITR | | Symmetric RIGHT modified ITR | |
|---|---|---|---|
| (modified 5′ ITR) | | (modified 3′ ITR) | |
| (ITR-33 left) | SEQ ID NO: 127 | (ITR-18, right) | SEQ ID NO: 110 |
| (ITR-34 left) | SEQ ID NO: 128 | (ITR-51, right) | SEQ ID NO: 125 |
| (ITR-35 left) | SEQ ID NO: 129 | (ITR-19, right) | SEQ ID NO: 111 |
| (ITR-36 left) | SEQ ID NO: 130 | (ITR-20, right) | SEQ ID NO: 112 |
| (ITR-37 left) | SEQ ID NO: 131 | (ITR-21, right) | SEQ ID NO: 113 |
| (ITR-38 left) | SEQ ID NO: 132 | (ITR-22 right) | SEQ ID NO: 114 |
| (ITR-39 left) | SEQ ID NO: 133 | (ITR-23, right) | SEQ ID NO: 115 |
| (ITR-40 left) | SEQ ID NO: 134 | (ITR-24, right) | SEQ ID NO: 116 |
| (ITR-41 left) | SEQ ID NO: 135 | (ITR-25 right) | SEQ ID NO: 117 |
| (ITR-42 left) | SEQ ID NO: 136 | (ITR-26 right) | SEQ ID NO: 118 |
| (ITR-43 left) | SEQ ID NO: 137 | (ITR-27 right) | SEQ ID NO: 119 |
| (ITR-44 left) | SEQ ID NO: 138 | (ITR-28 right) | SEQ ID NO: 120 |
| (ITR-45 left) | SEQ ID NO: 139 | (ITR-29, right) | SEQ ID NO: 121 |
| (ITR-46 left) | SEQ ID NO: 140 | (ITR-30, right) | SEQ ID NO: 122 |
| (ITR-47, left) | SEQ ID NO: 141 | (ITR-31, right) | SEQ ID NO: 123 |
| (ITR-48, left) | SEQ ID NO: 142 | (ITR-32 right) | SEQ ID NO: 124 |

In some embodiments, a cleavable ceDNA comprising an asymmetric ITR pair can comprise an ITR with a modification corresponding to any of the modifications in ITR sequences or ITR partial sequences shown in any one or more of Tables 4A-4B herein, or disclosed in Tables 2, 3, 4, 5, 6, 7, 8, 9 or 10A-10B of PCT/US18/49996 filed Sep. 7, 2018 which is incorporated herein in its entirety by reference.

### VI. Cleavable ceDNA and Exemplary Gene Editing Systems

As described above, the present disclosure describes the engineering of gRNA target sequences (gRNA TS) and protospacer adjacent motifs (PAMs), and optional spacer sequences located adjacent to gRNA TS/PAM, within ceDNA to generate cleavable ceDNA constructs. After processing of the cleavable ceDNA to generate cleaved ceDNA, the cleaved ceDNA can be used as a repair or donor sequence for gene editing purposes. In particular, the cleaved ceDNA can be used as the repair template or donor sequence in a gene editing system which further comprises a nuclease, e.g., a Cas9 enzyme, and at least one gRNA molecule to edit a target sequence.

According to some aspects, the disclosure provides a cleavable ceDNA comprising a first inverted terminal repeat (ITR); a first guide RNA (gRNA) target sequence (TS); a first protospacer adjacent motif (PAM); at least one transgene cassette; a second protospacer adjacent motif (PAM); a second gRNA target sequence (TS); and a second ITR. Optionally, one or more spacers can be placed immediately adjacent to any of the gRNA TS/PAM sequences. See, e.g., FIGS. 1A-1D for exemplary cleavable ceDNA constructs.

According to some aspects, the disclosure provides a cleavable ceDNA comprising a first inverted terminal repeat (ITR); a first guide RNA (gRNA) target sequence (TS): a first protospacer adjacent motif (PAM); at least one transgene cassette: a second protospacer adjacent motif (PAM); a second gRNA target sequence (TS); a third gRNA target sequence (TS); a third protospacer adjacent motif (PAM); and a second ITR. In one embodiment, the third gRNA TS is placed within the at least one transgene cassette. Optionally, one or more spacers (“stuffer”) can be placed immediately adjacent to any of the first, second, or third gRNA TS/PAM unit. See, e.g., FIGS. 1A-1D for exemplary cleavable ceDNA constructs.

According to some aspects, a cleavable non-viral capsid-free closed-ended DNA (ceDNA) comprising in the following order a first inverted terminal repeat (ITR); a first guide RNA (gRNA) target sequence (TS); a first protospacer adjacent motif (PAM); at least one transgene cassette; a second protospacer adjacent motif (PAM); a second gRNA target sequence (TS); and a second ITR. Optionally, the cleavable non-viral capsid-free ceDNA can further comprise a third guide RNA (gRNA) target sequence (TS) and a third protospacer adjacent motif (PAM) within the at least one transgene cassette.

It is a finding of the present disclosure that cleavable ceDNA was effectively cleaved by a nuclease enzyme, such as Cas9, and that this reaction was more efficient when a spacer sequence was included between a matching gRNA TS and PAM (gRNA TS/PAM unit) and at least one ITR of the cleavable ceDNA.

In certain embodiments, one spacer (also referred to as “stuffer”) is inserted in the cleavable ceDNA. For example, a first spacer can be placed between a first ITR and a first gRNA TS/PAM unit; or between a second gRNA TS/PAM unit and a second ITR. In certain embodiments, two spacers are inserted in the cleavable ceDNA, e.g., a first spacer placed between the first ITR and the first gRNA TS/PAM unit and a second spacer placed between the second gRNA TS/PAM unit and the second ITR. In certain embodiments, three spacers can be inserted in the cleavable ceDNA, e.g., a first spacer placed between the first ITR and the first gRNA TS/PAM unit; a second spacer placed between the second gRNA TS/PAM unit and the second ITR; and a third spacer placed immediately adjacent to the third gRNA TS/PAM unit. In certain embodiments, four spacers are inserted in the cleavable ceDNA, e.g., a first spacer placed between the first ITR and the first gRNA TS/PAM unit; a second spacer placed between the second gRNA TS/PAM unit and the second ITR; and a third spacer placed immediately adjacent to third gRNA TS/PAM unit present within at least one transgene cassette; and a fourth spacer placed between either the first gRNA TS/PAM or the second gRNA TS/PAM and the at least one transgene cassette. See, e.g., FIGS. 1A-1D for exemplary cleavable ceDNA constructs comprising various numbers of spacers (also referred to as “stuffers”) in various locations in the cleavable ceDNA, immediately adjacent to one or more gRNA TS/PAM unit(s).

Accordingly, in some embodiments, the cleavable ceDNA comprises a first (stuffer) spacer sequence between the first ITR and the first gRNA TS. According to some embodiments, the first spacer sequence is between 1-200 nucleotides in length, for example between 1-175, between 1-150, between 1-100, between 50-150, between 150-200, between 100-200, between 50-100, between 25-50 nucleotides in length. According to some embodiments, the first spacer sequence is about 10, about 25, about 50, about 75, about 100, about 125, about 150, about 175, about 200 or about 300 nucleotides in length. According to some embodiments, the second spacer sequence is about 50 nucleotides in length. According to further embodiments, the first spacer sequence is about 100 nucleotides in length. According to further embodiments, the second spacer sequence is about 150 nucleotides in length.

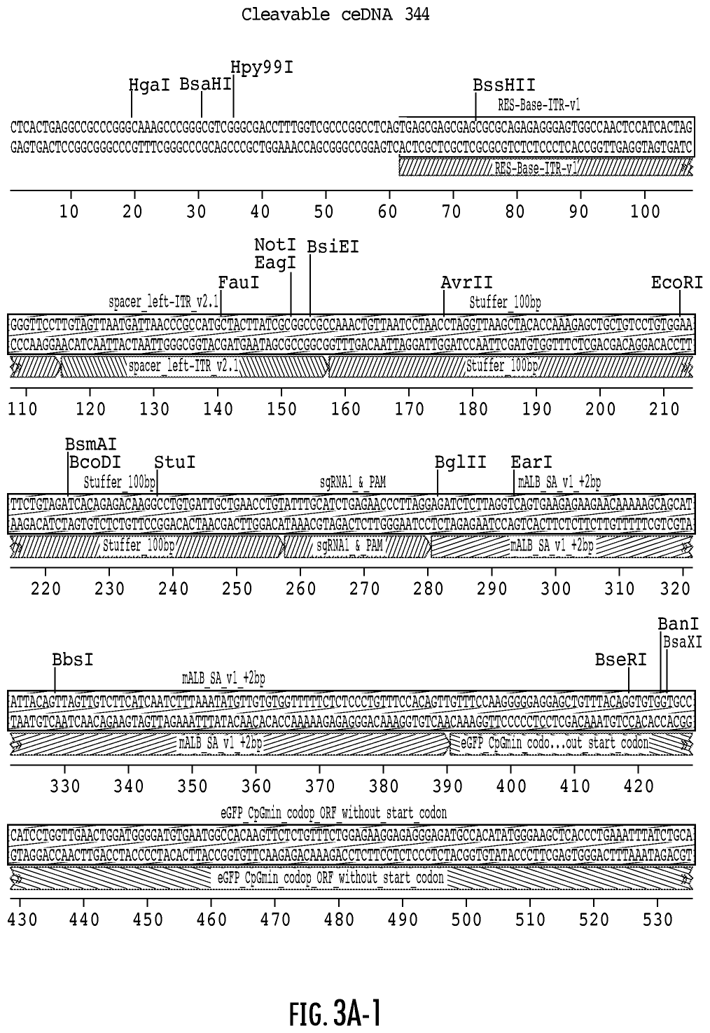

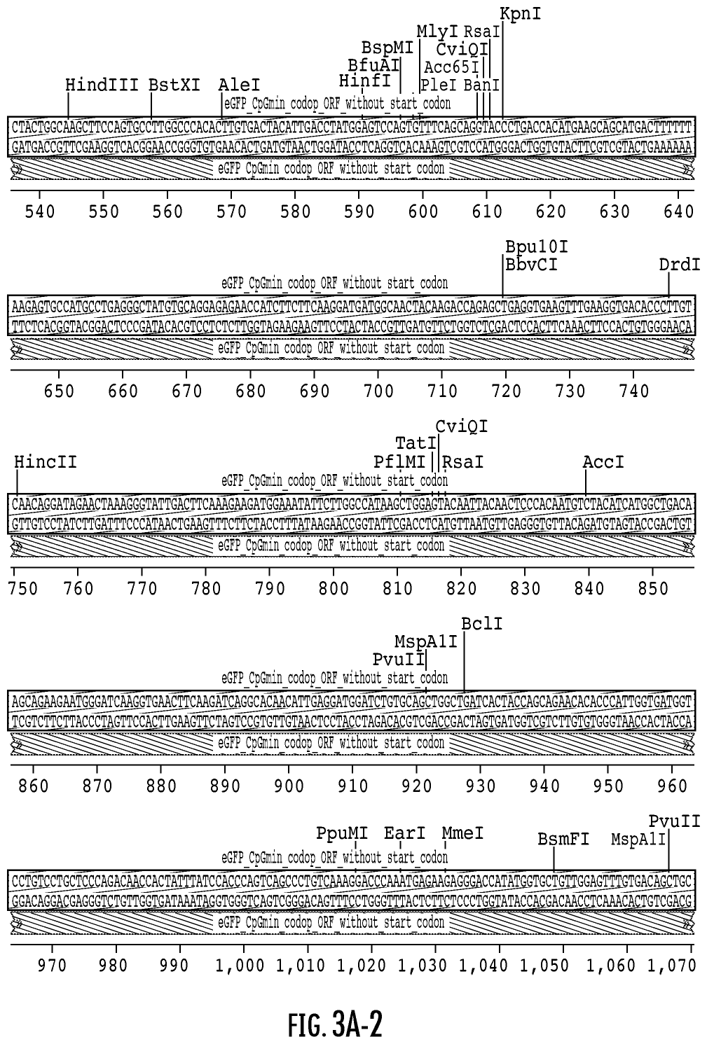

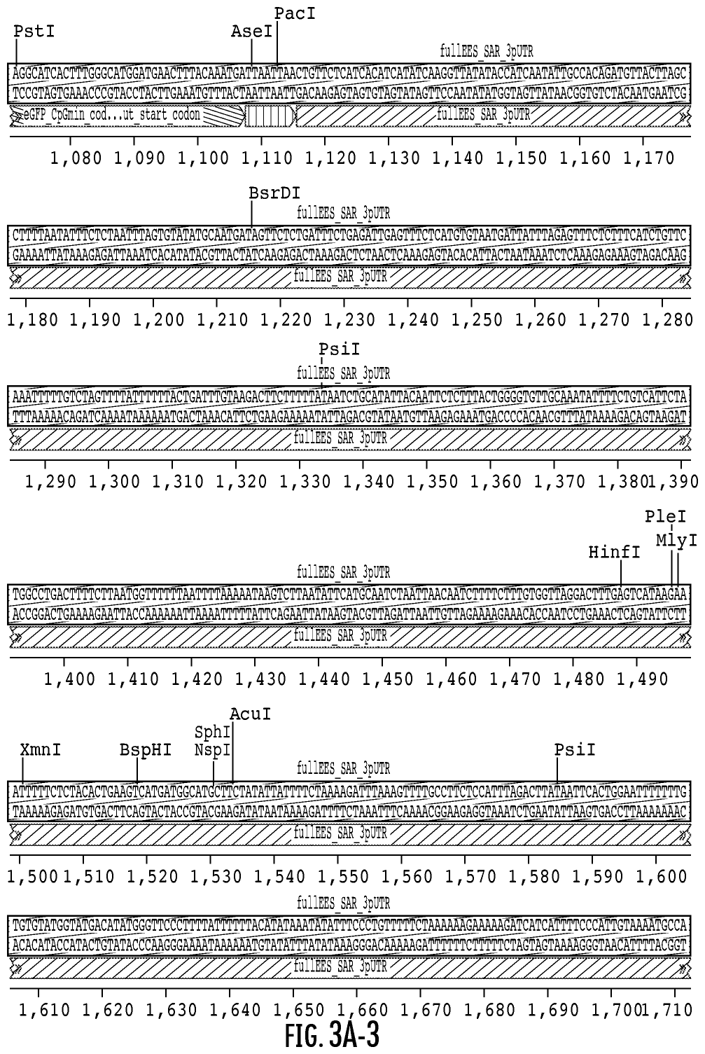

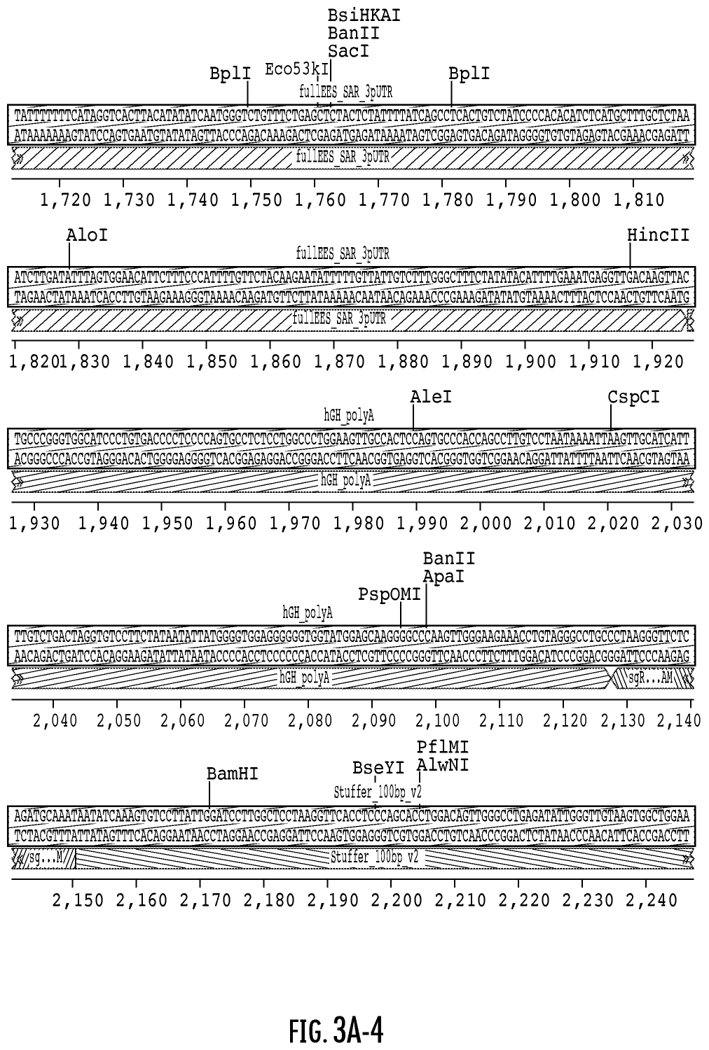

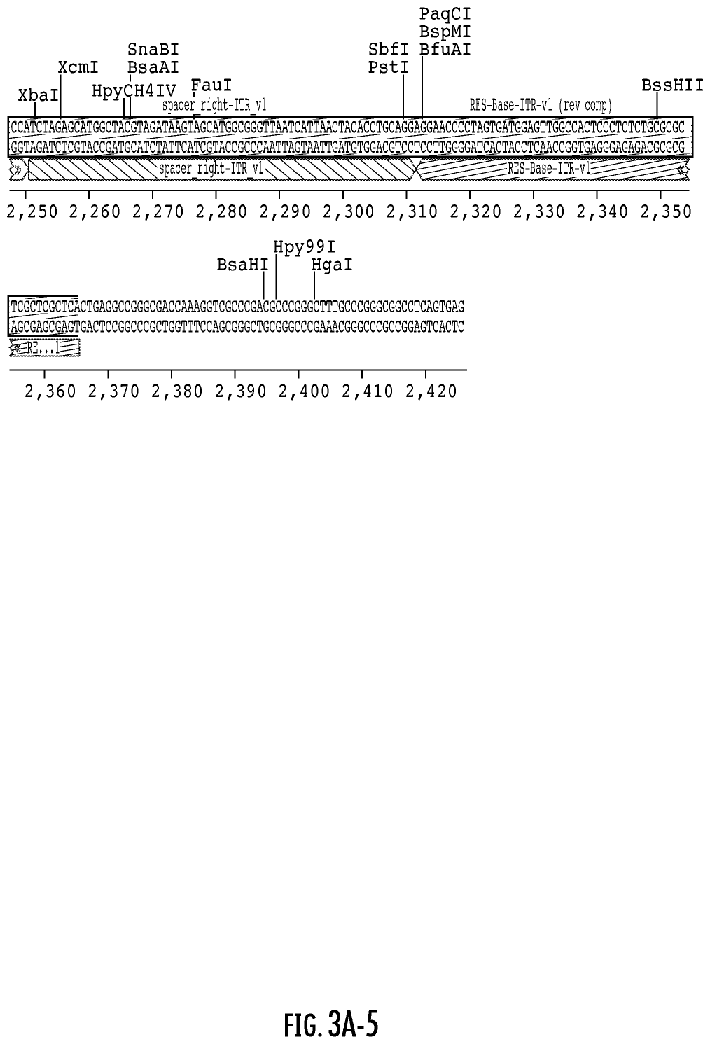

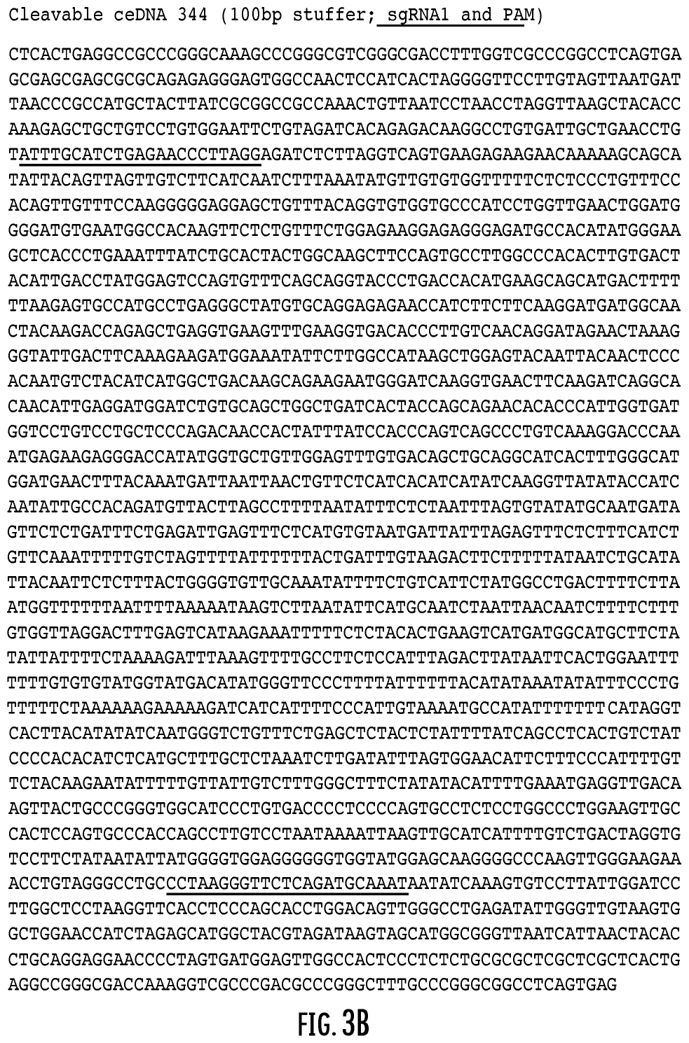

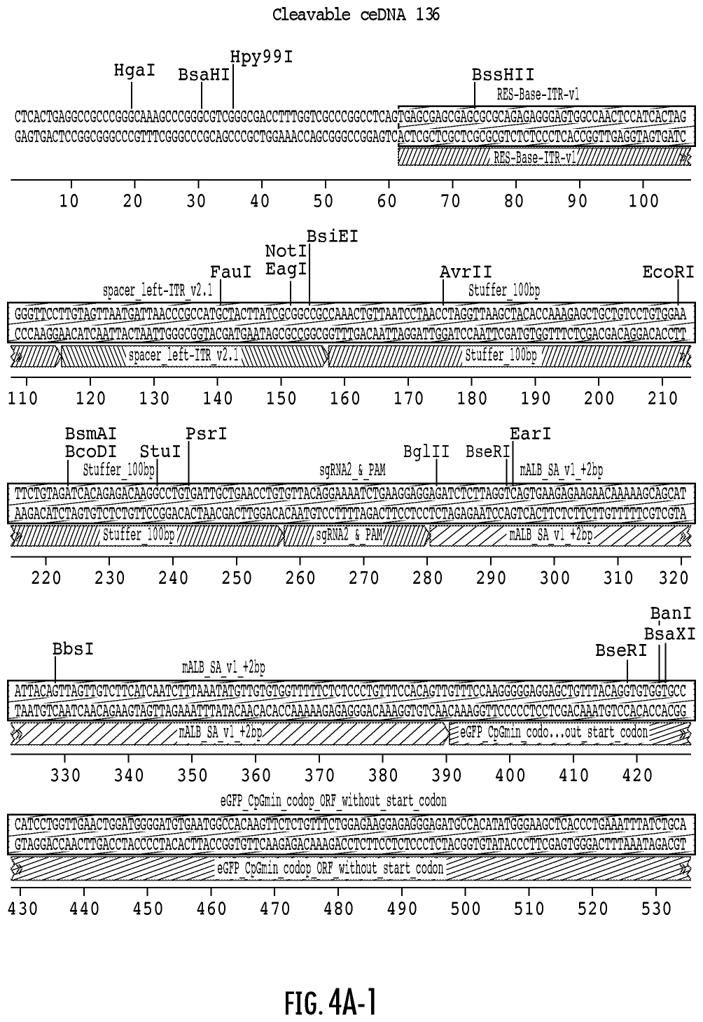

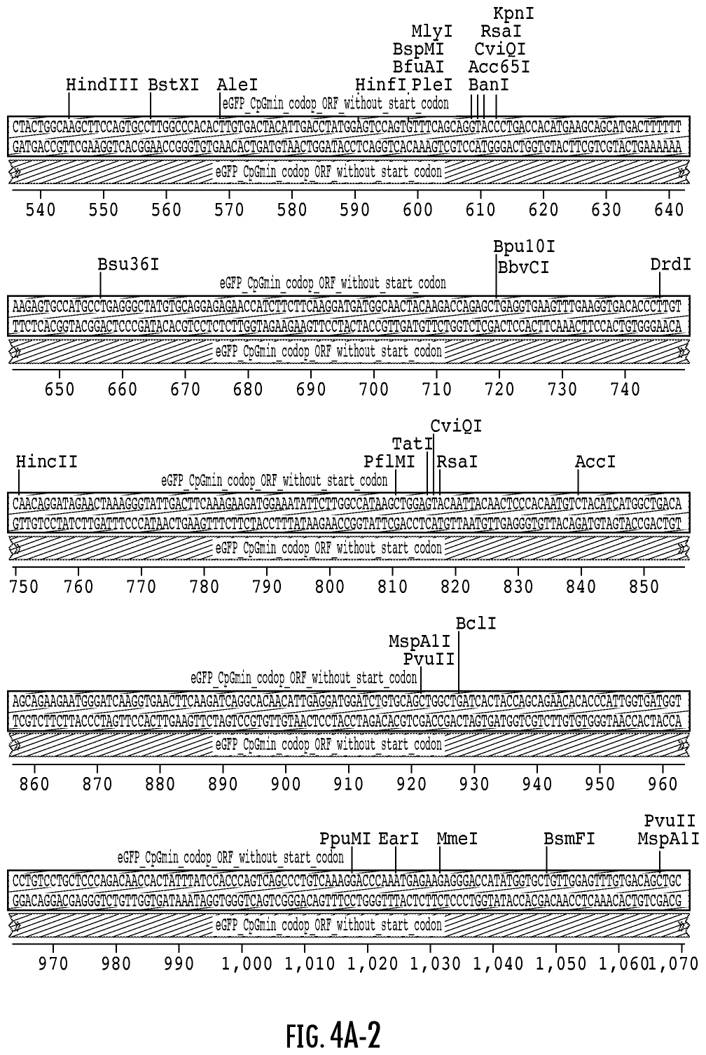

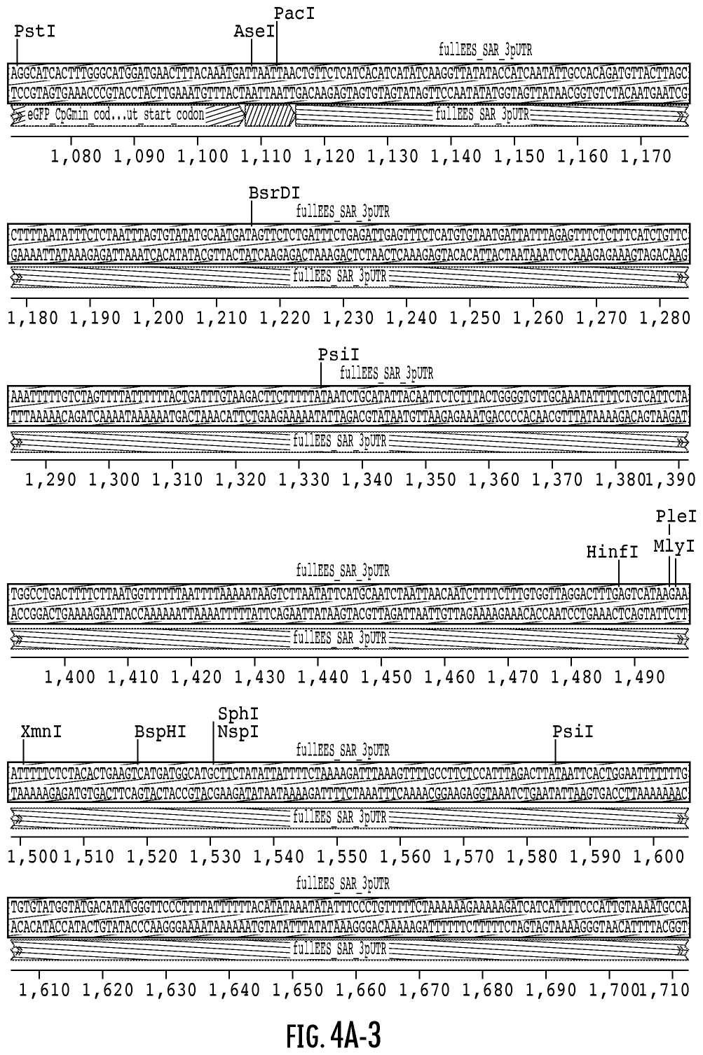

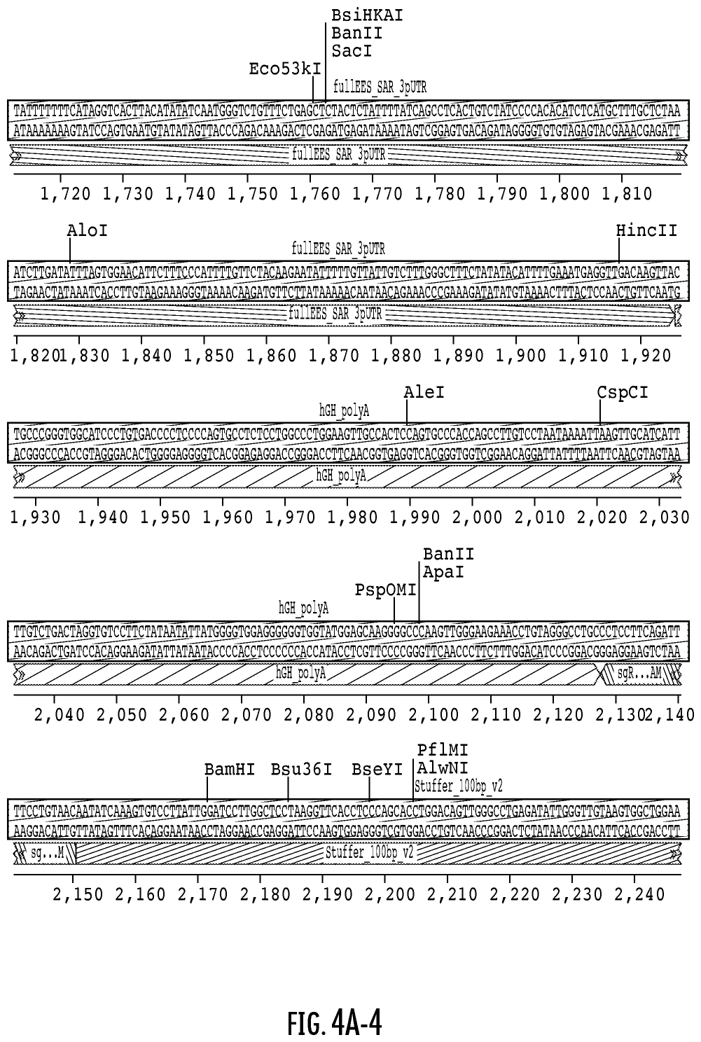

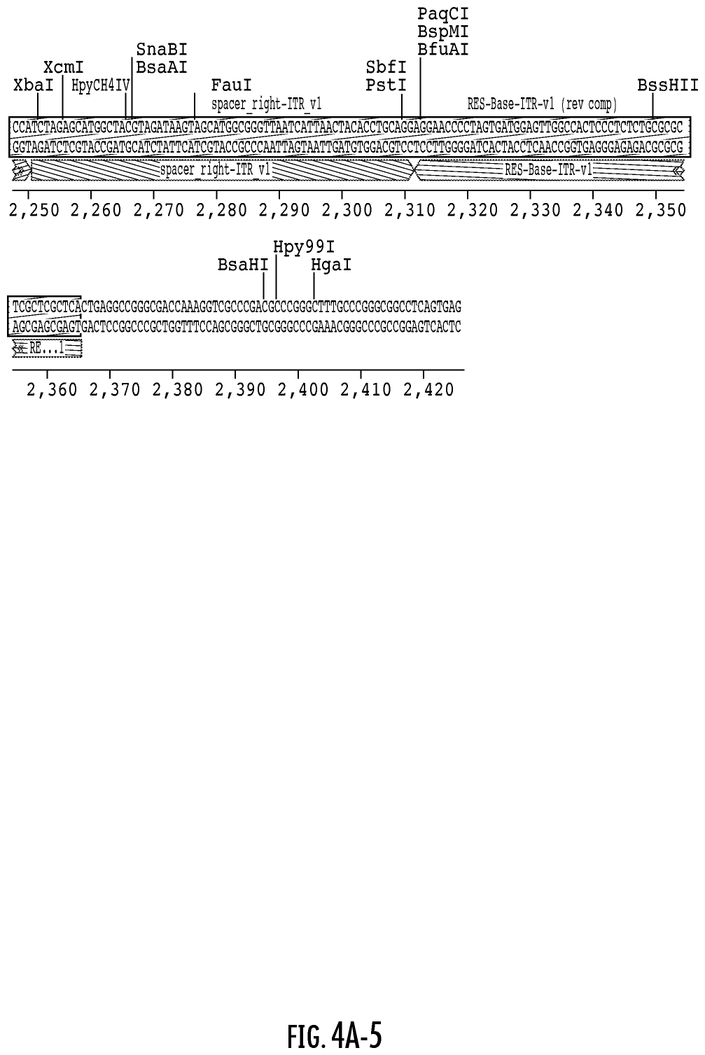

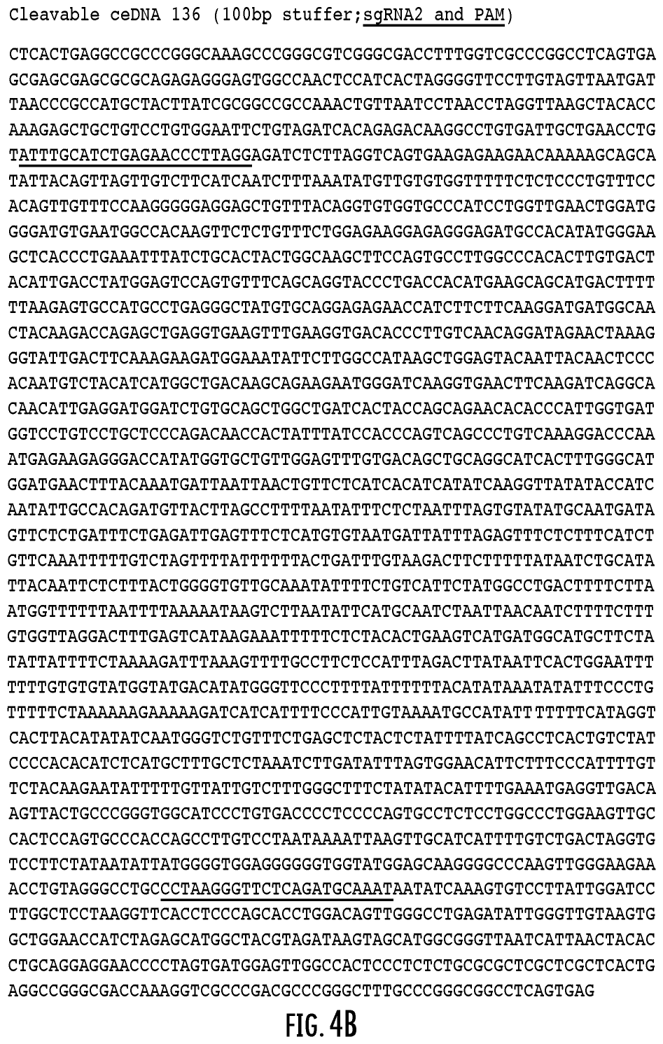

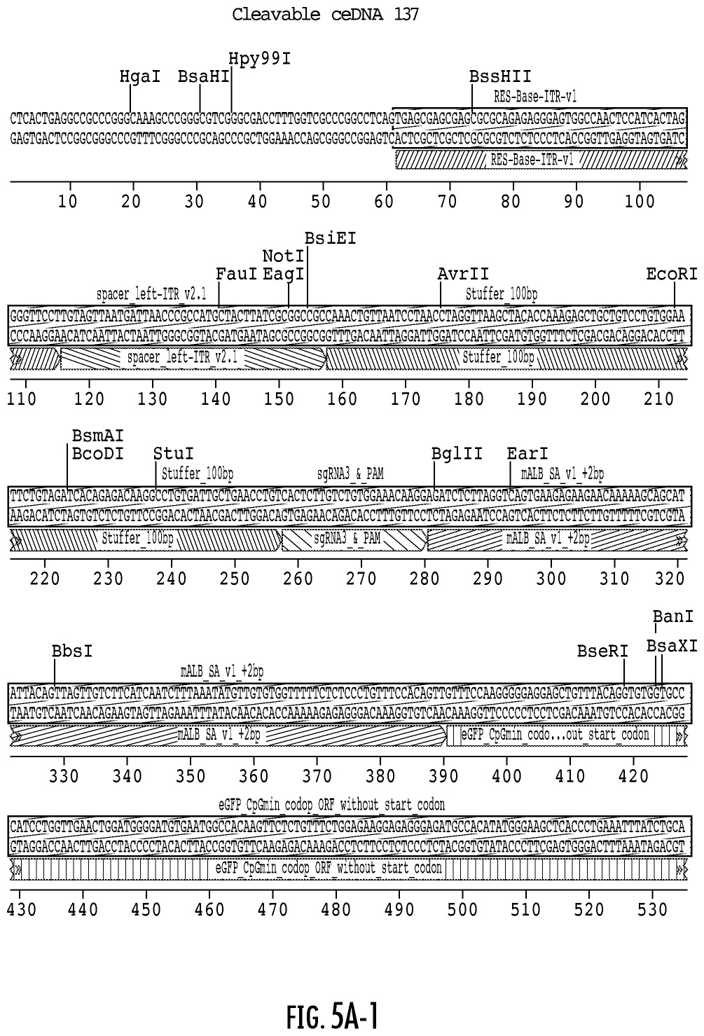

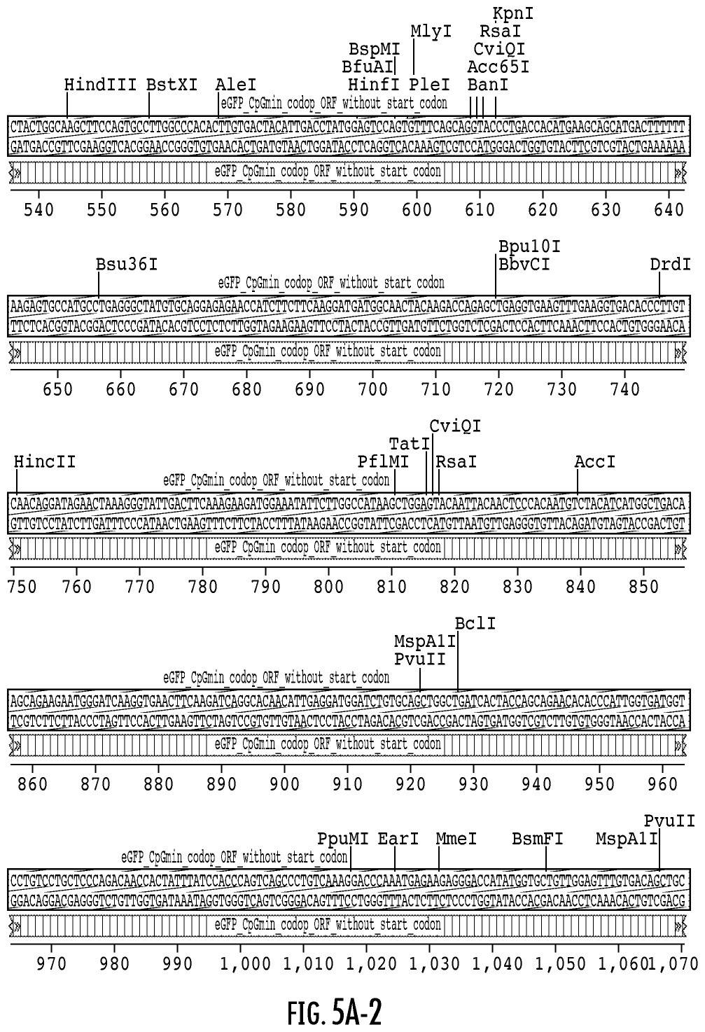

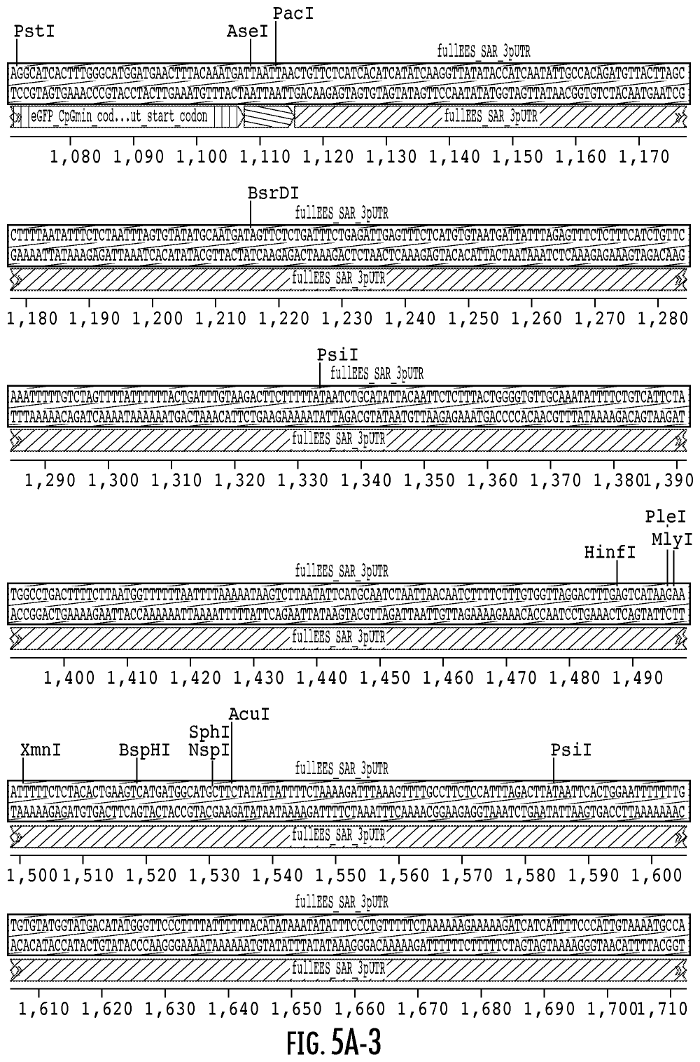

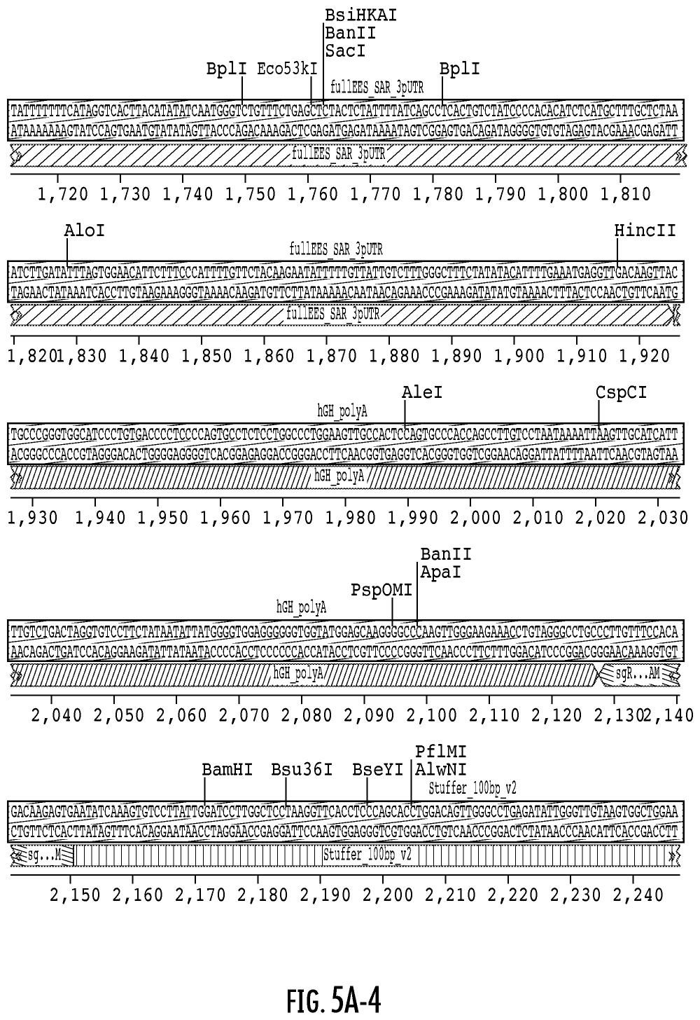

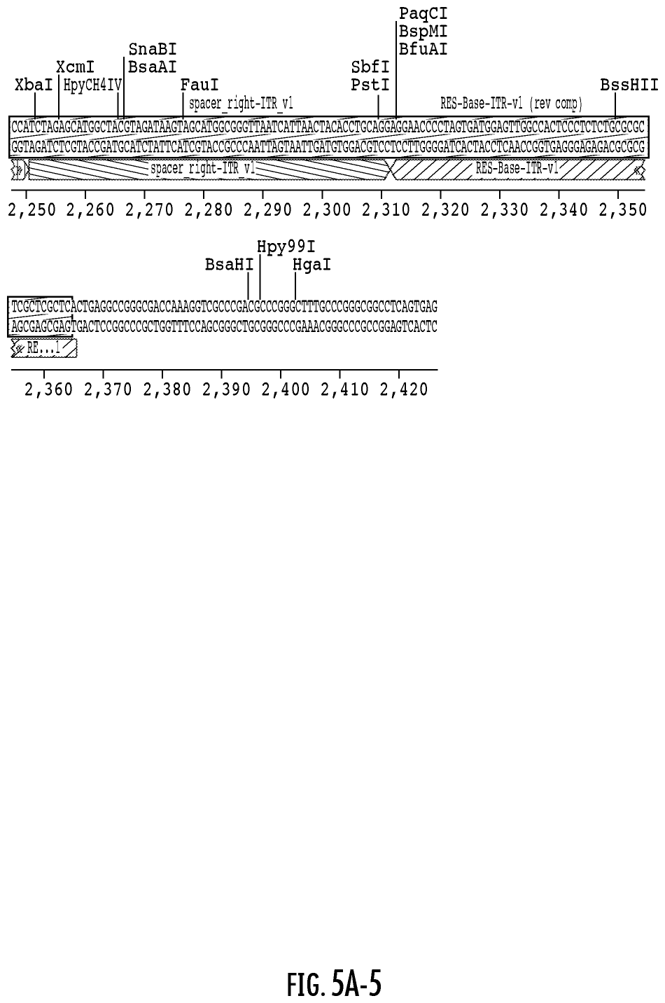

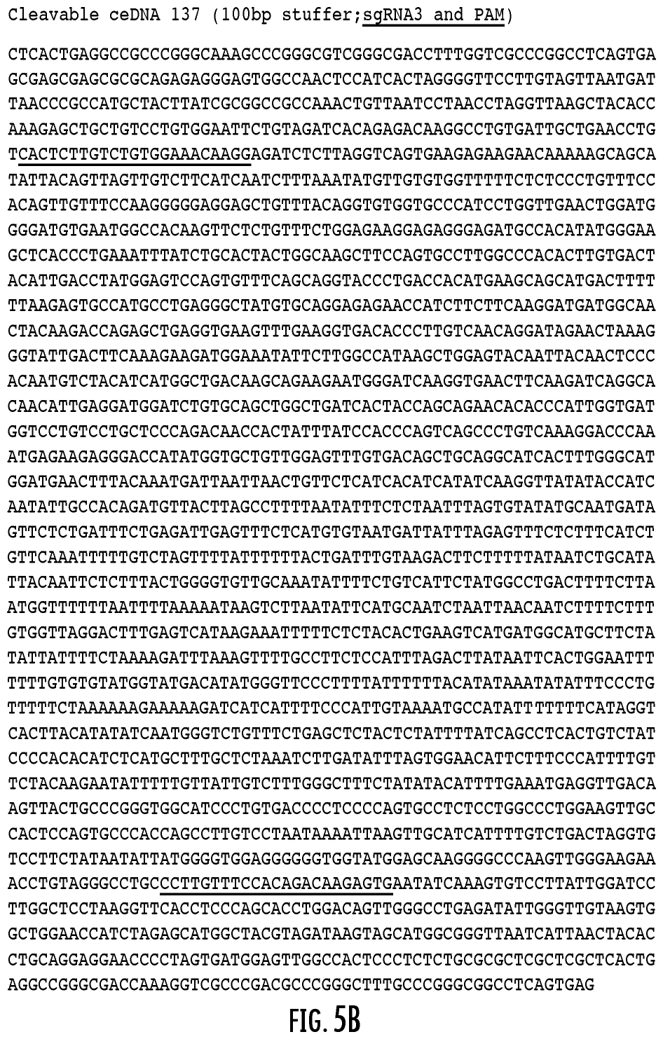

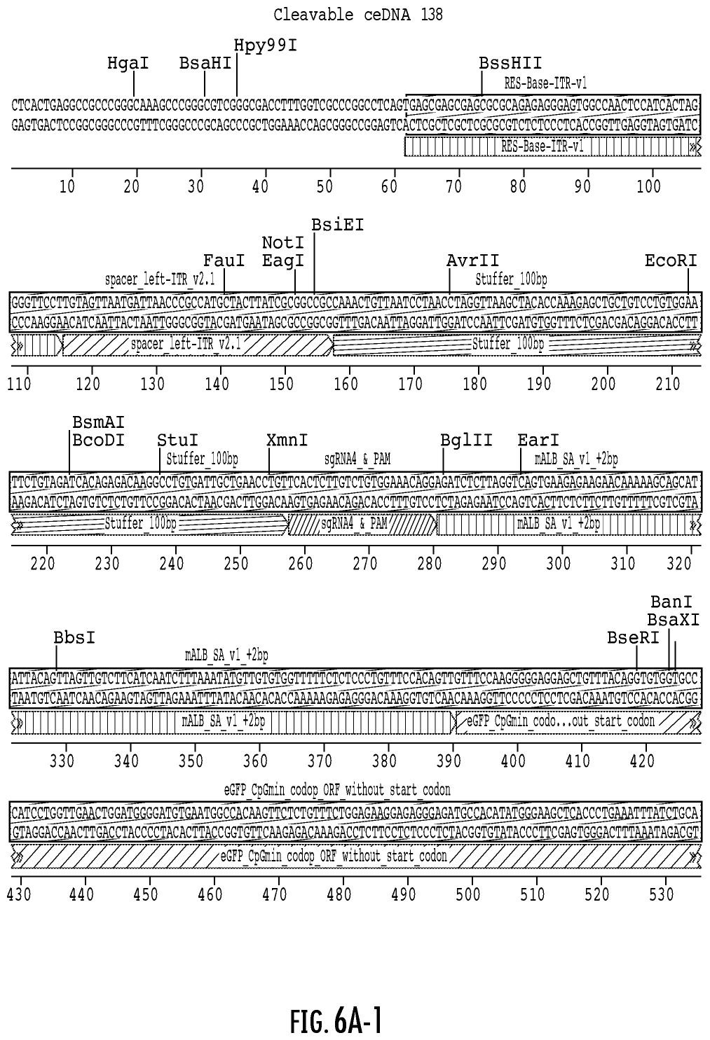

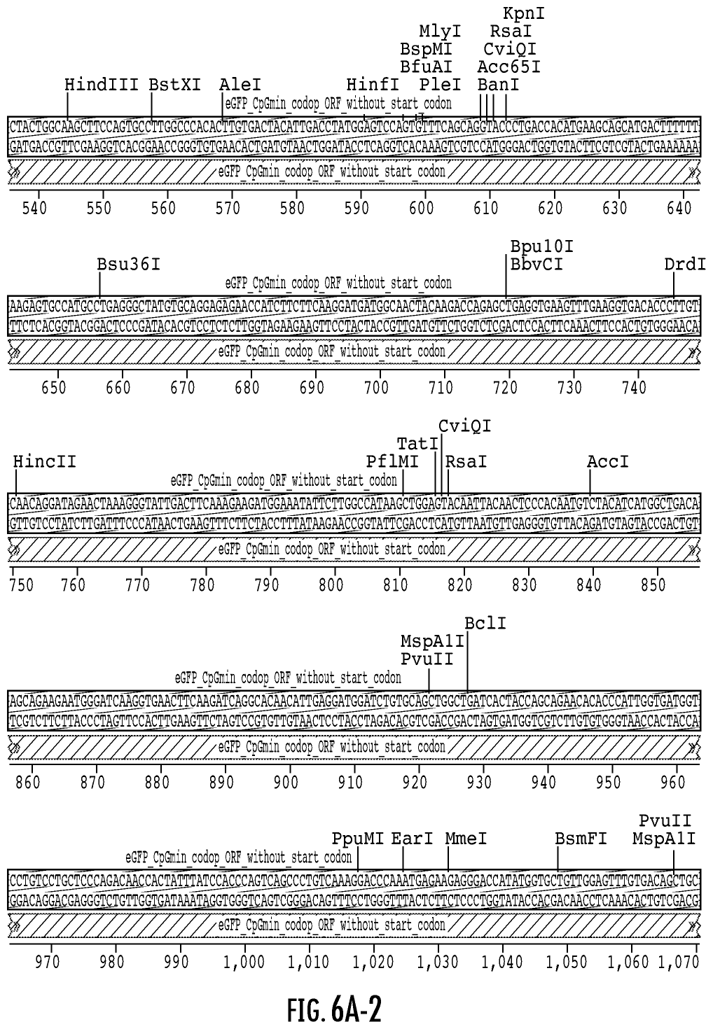

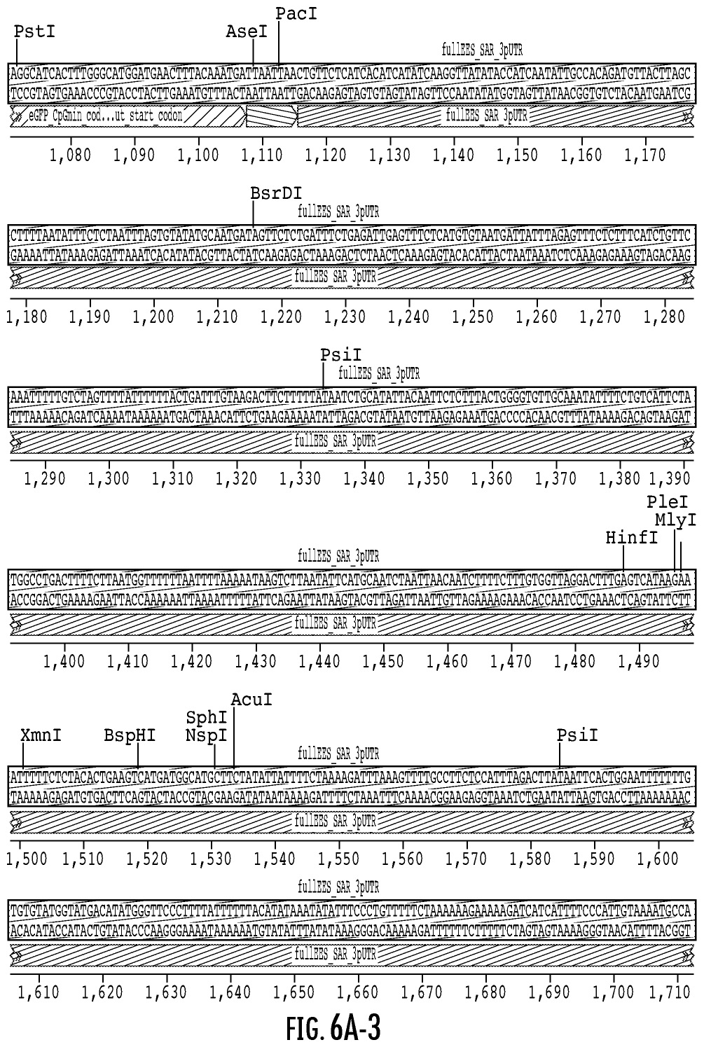

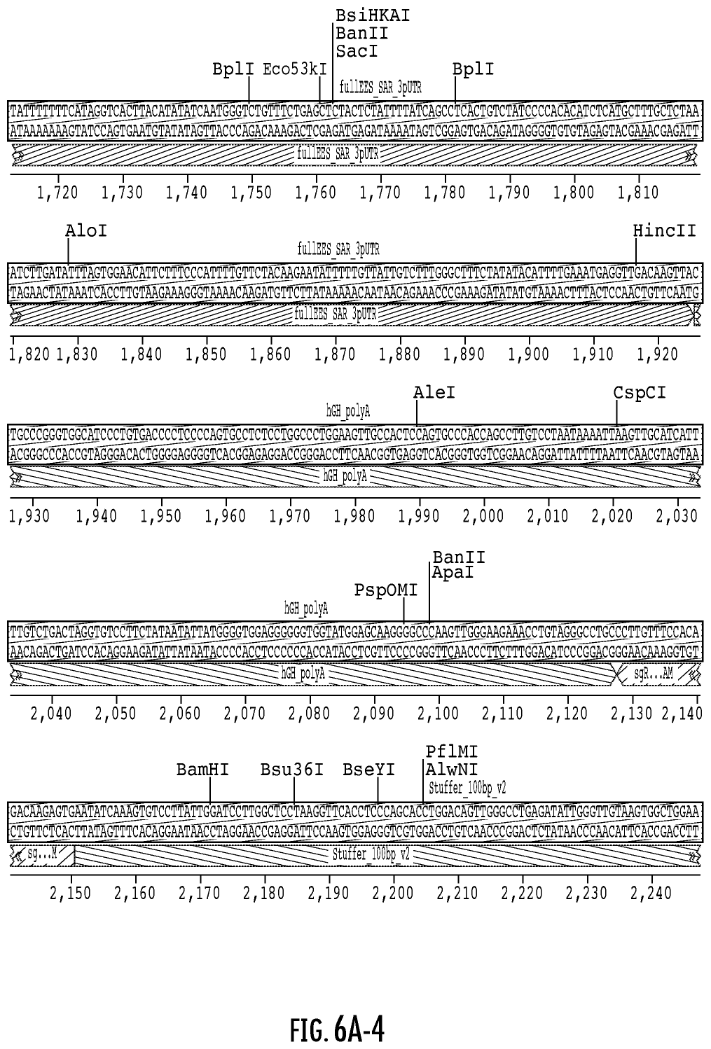

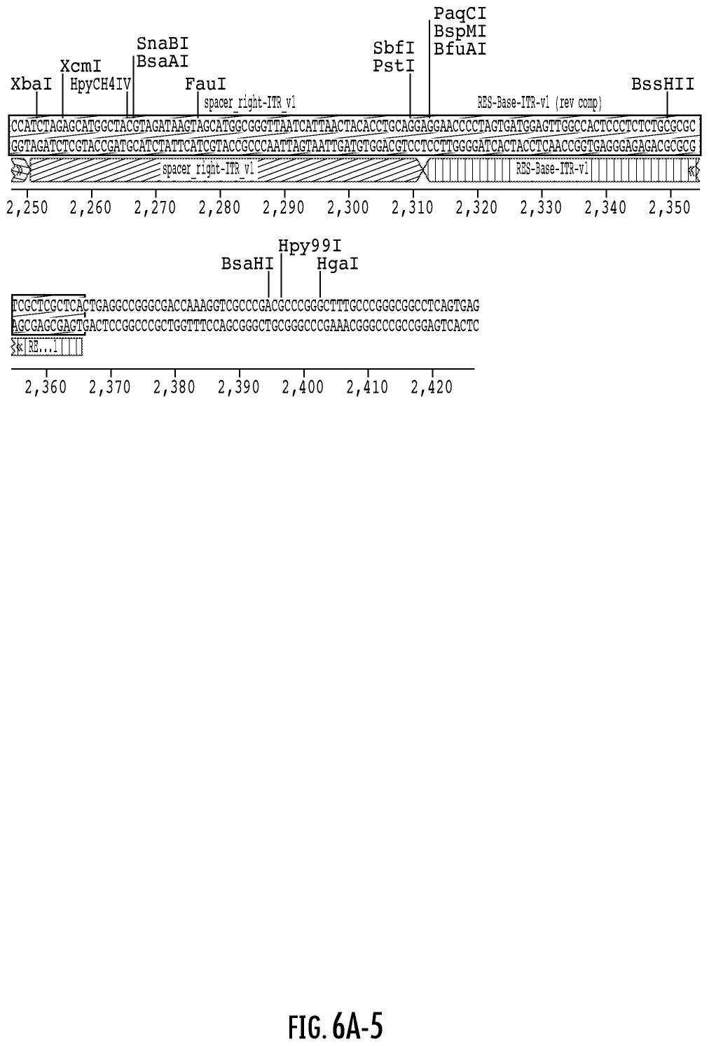

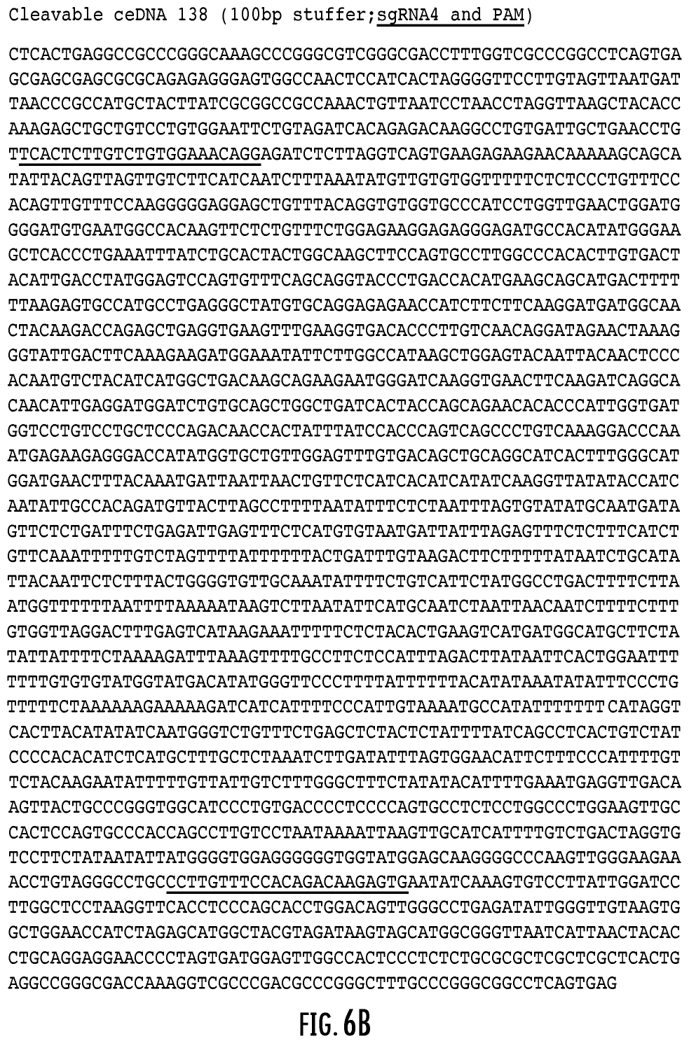

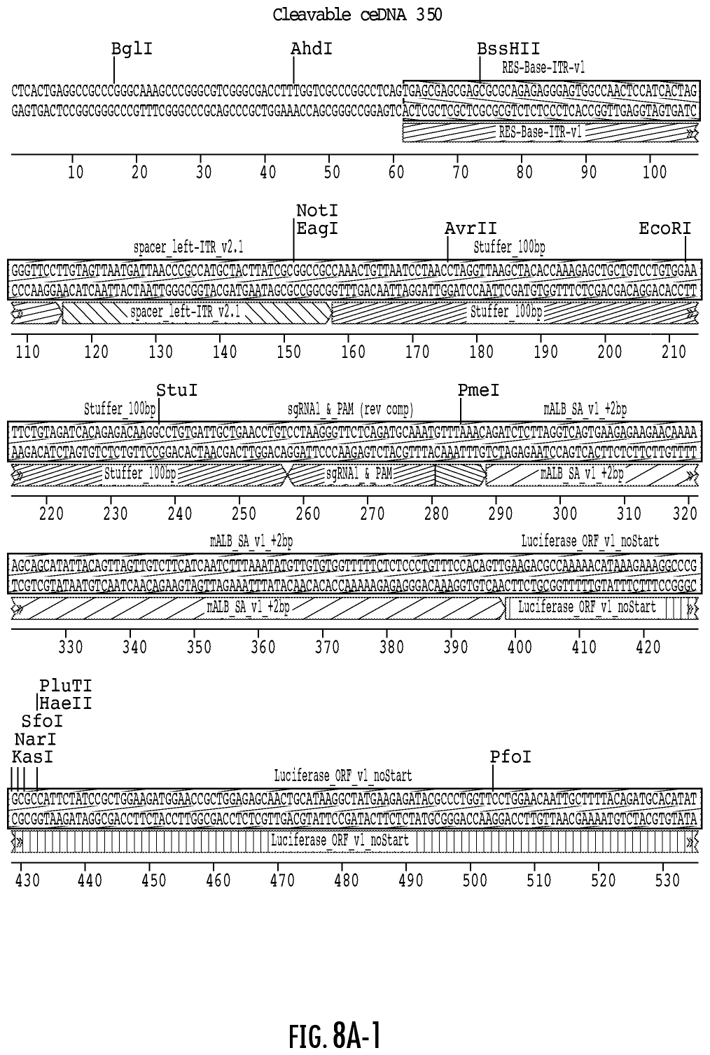

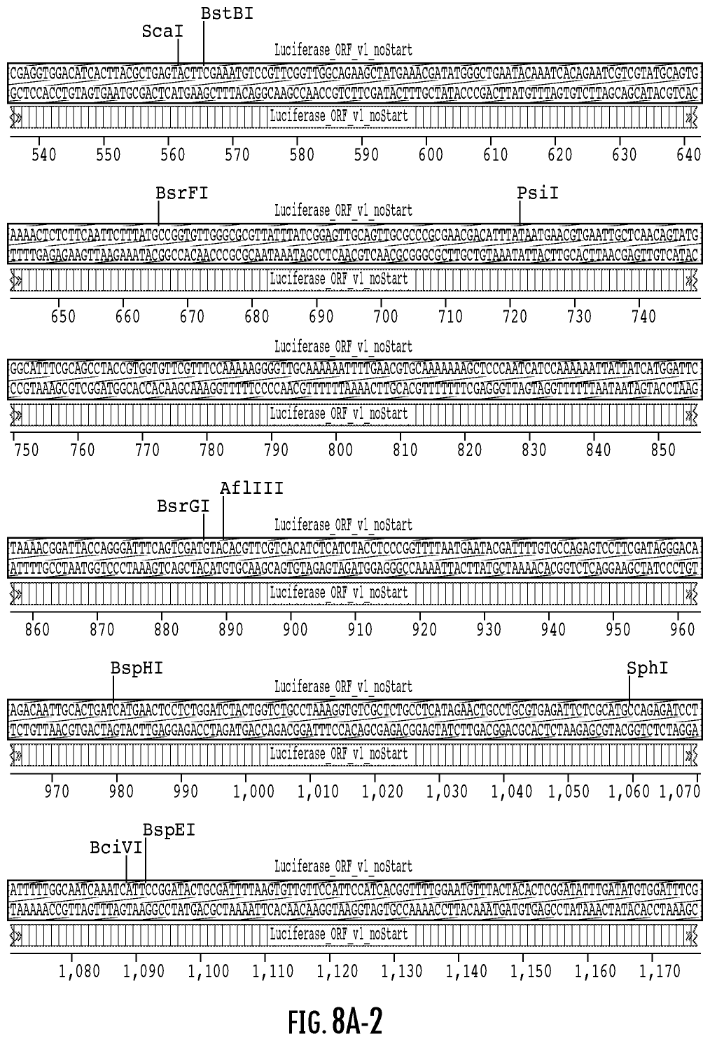

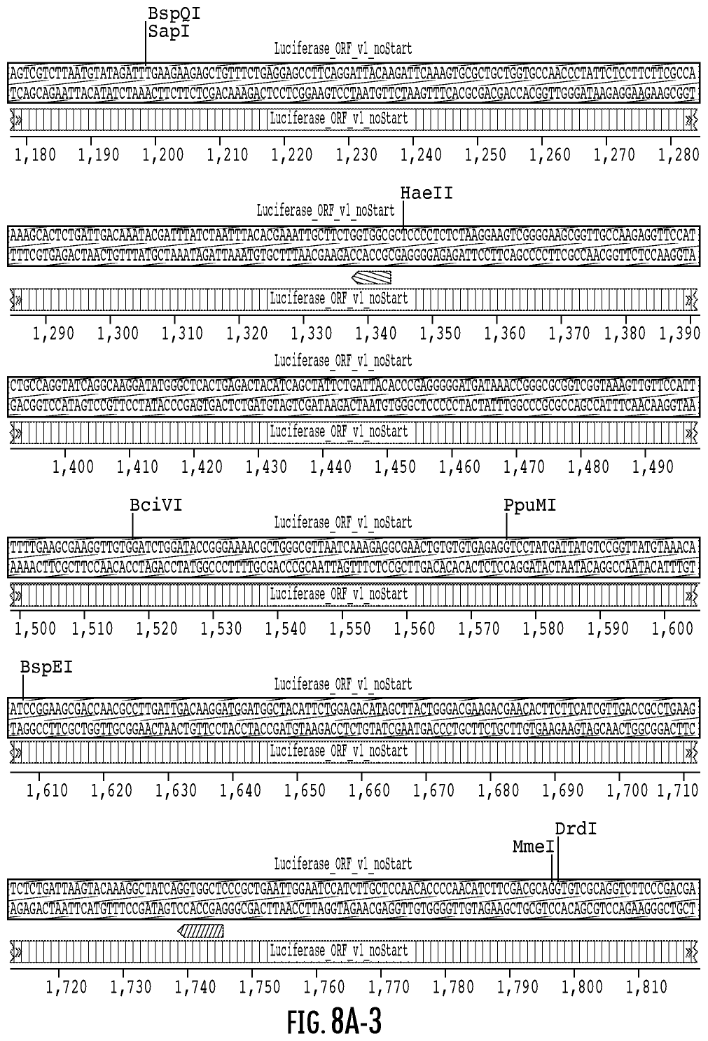

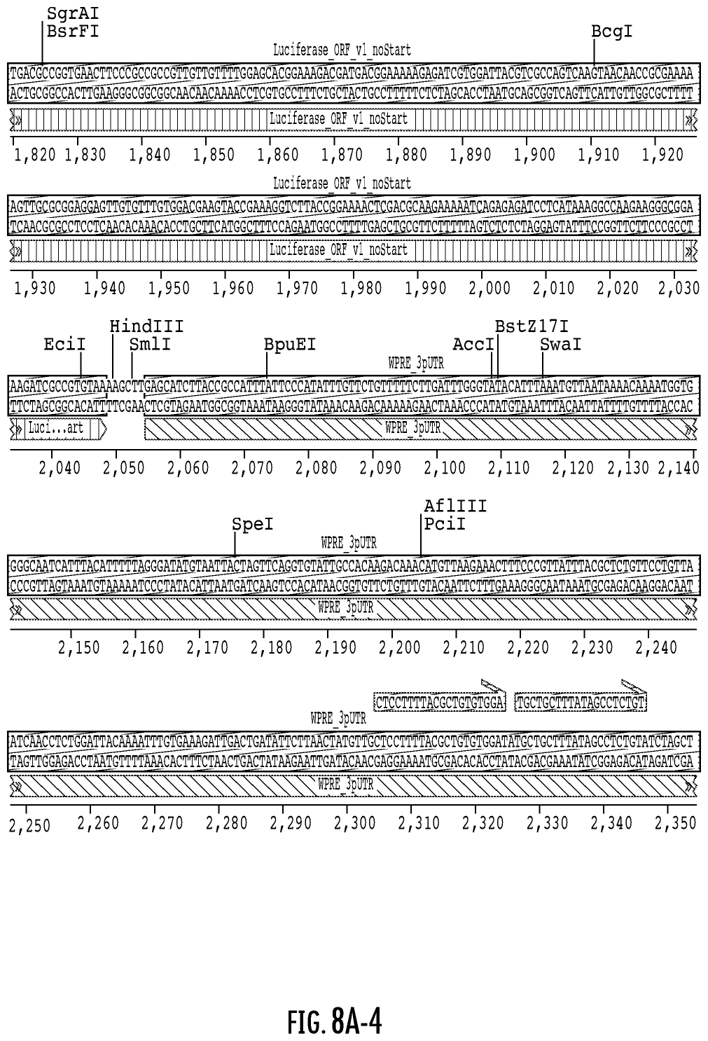

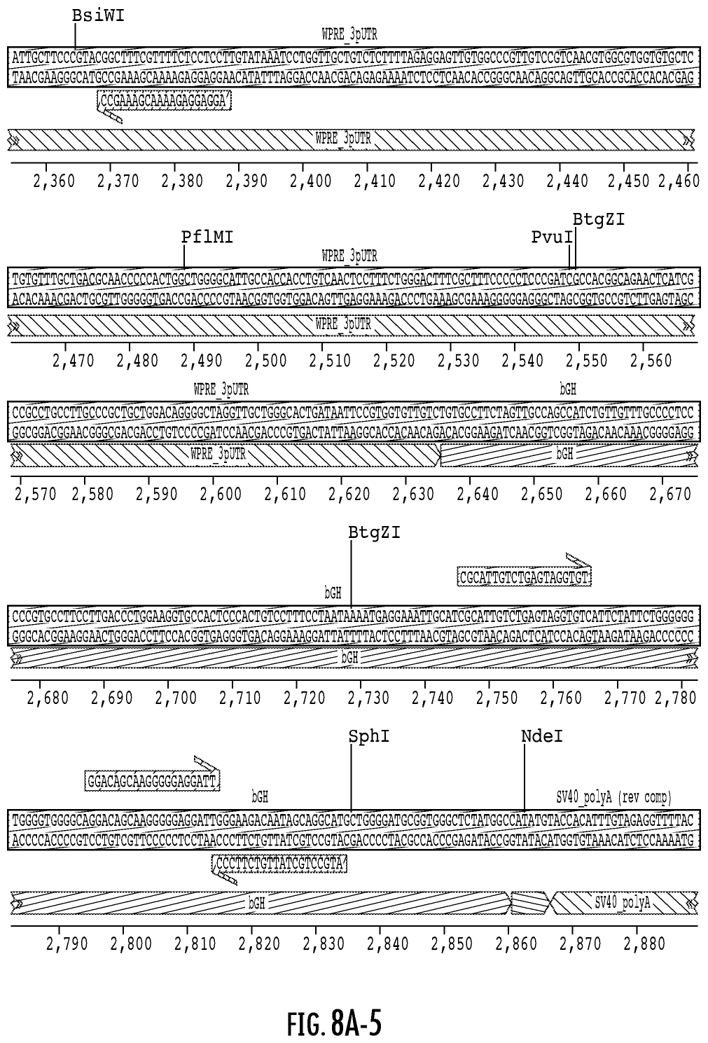

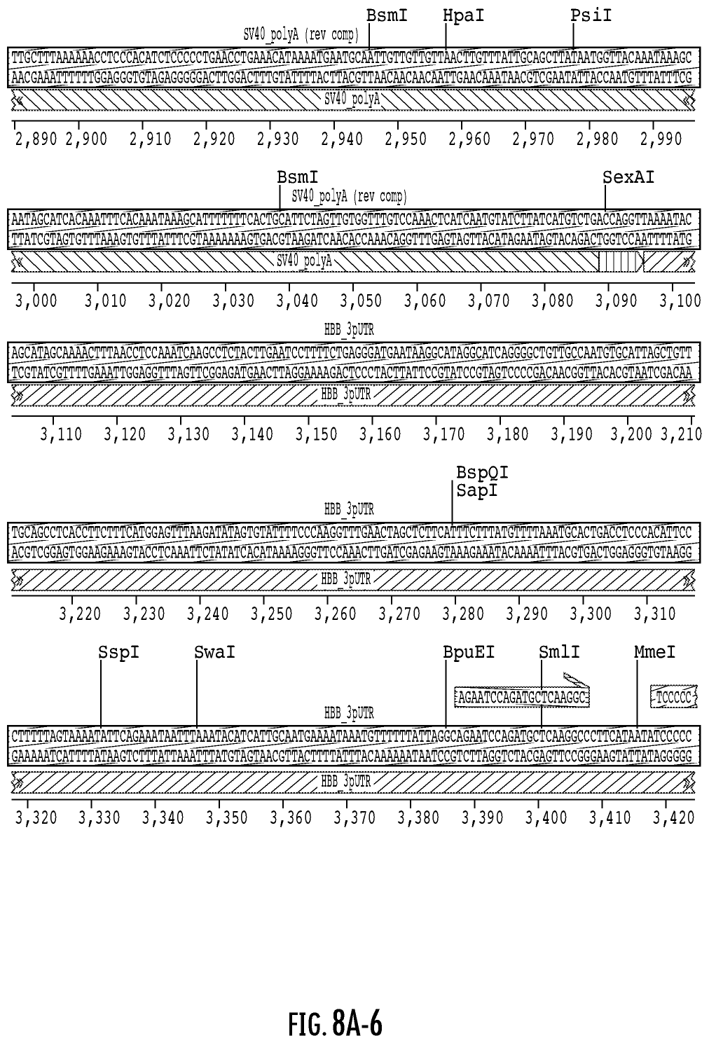

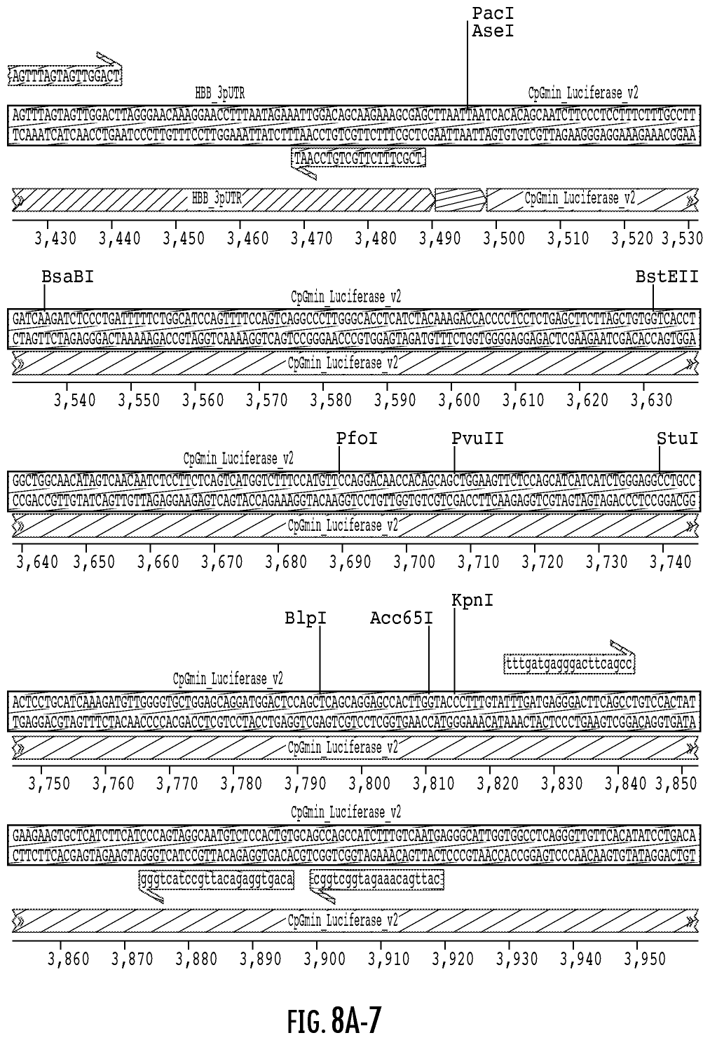

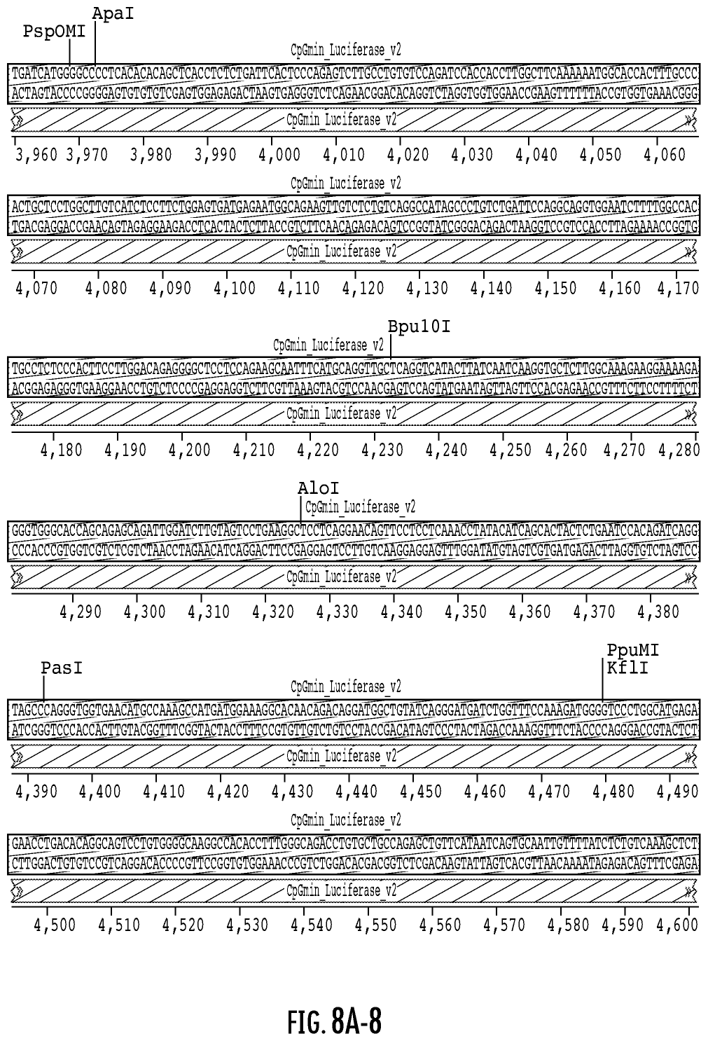

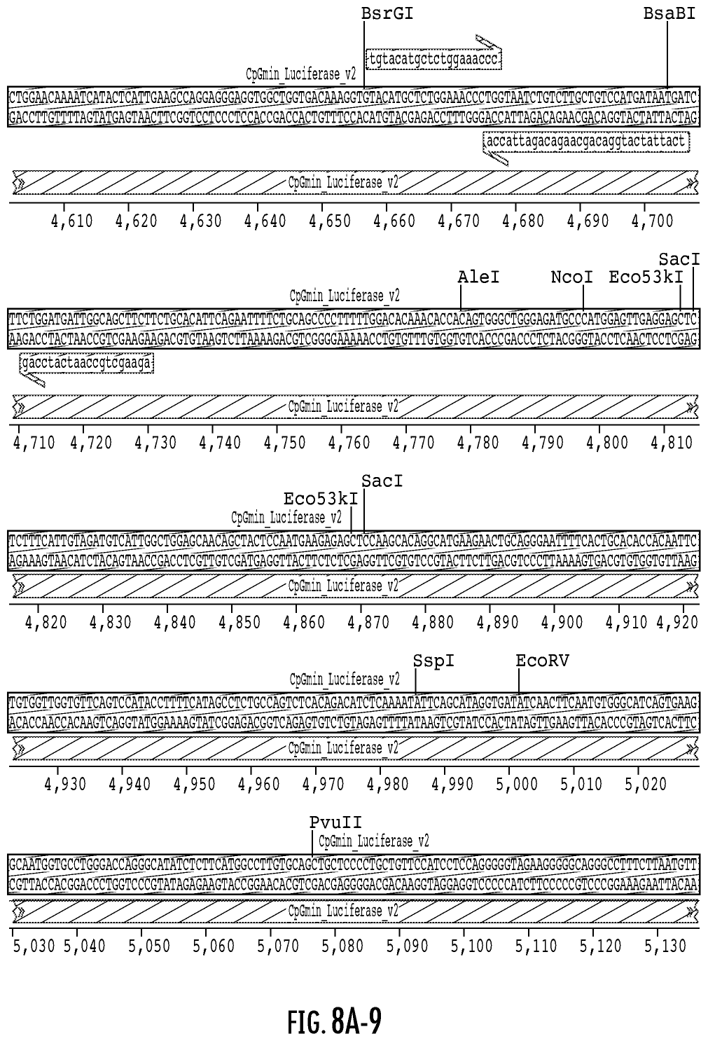

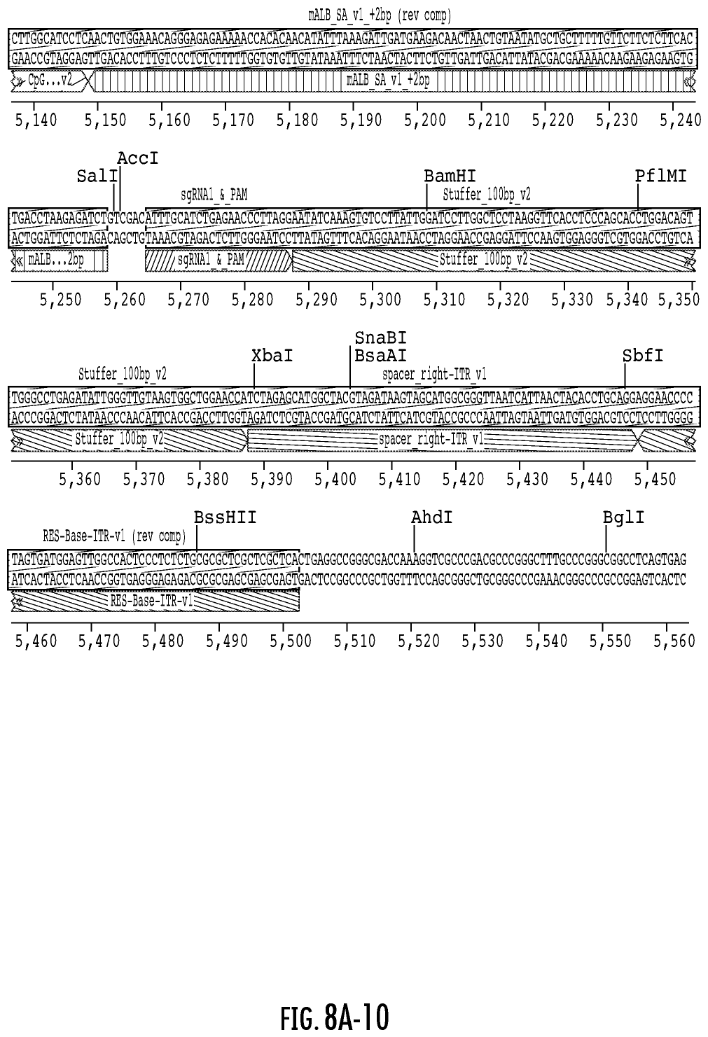

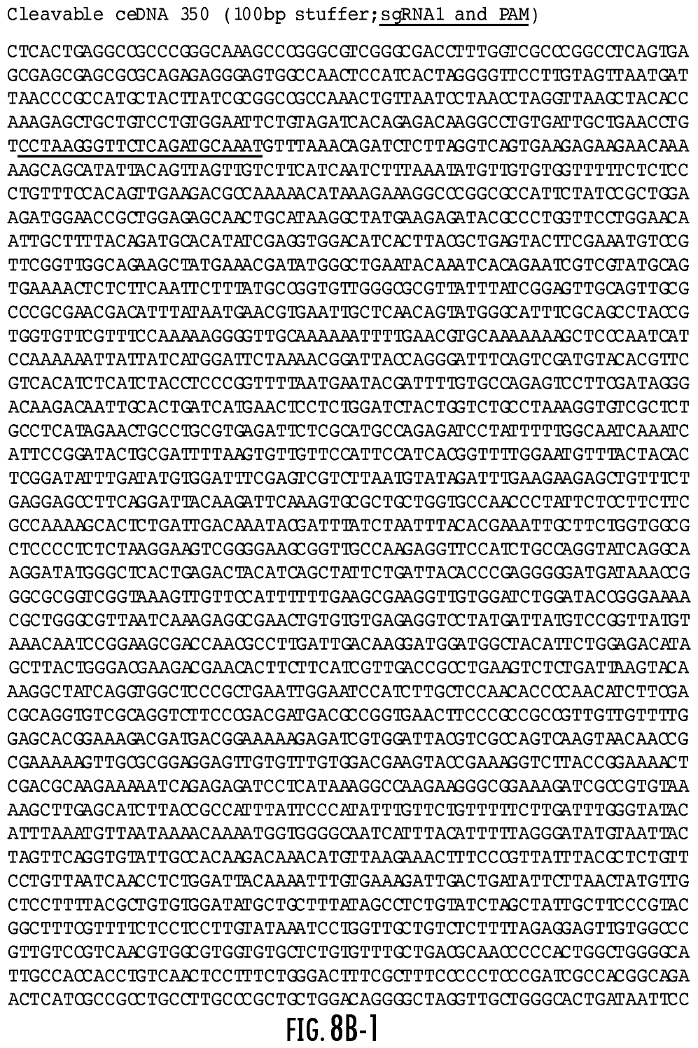

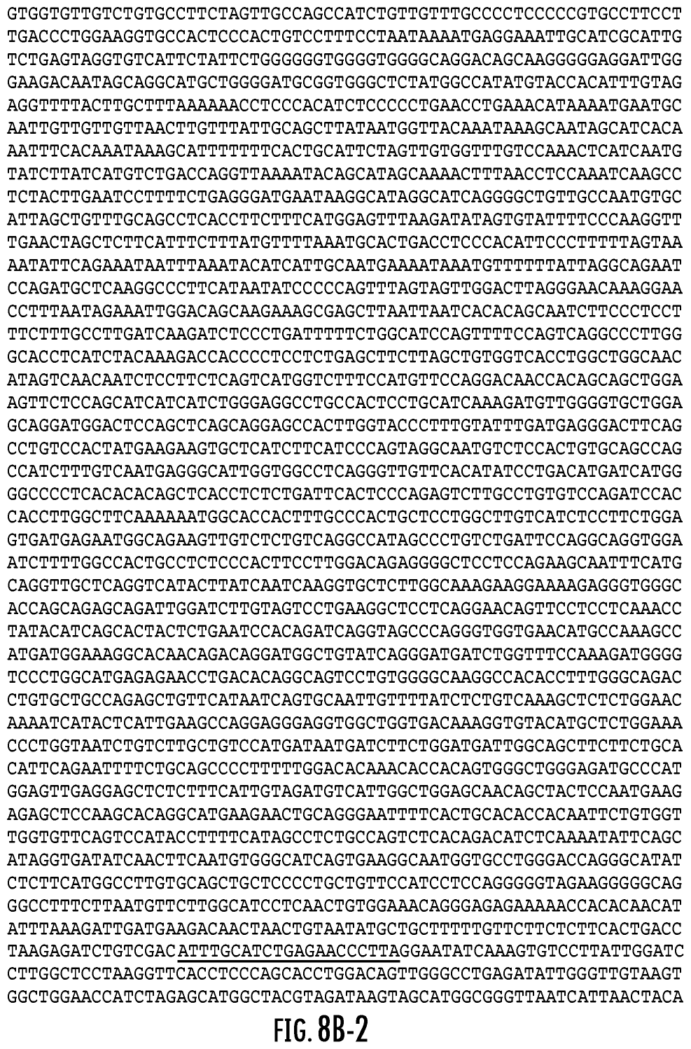

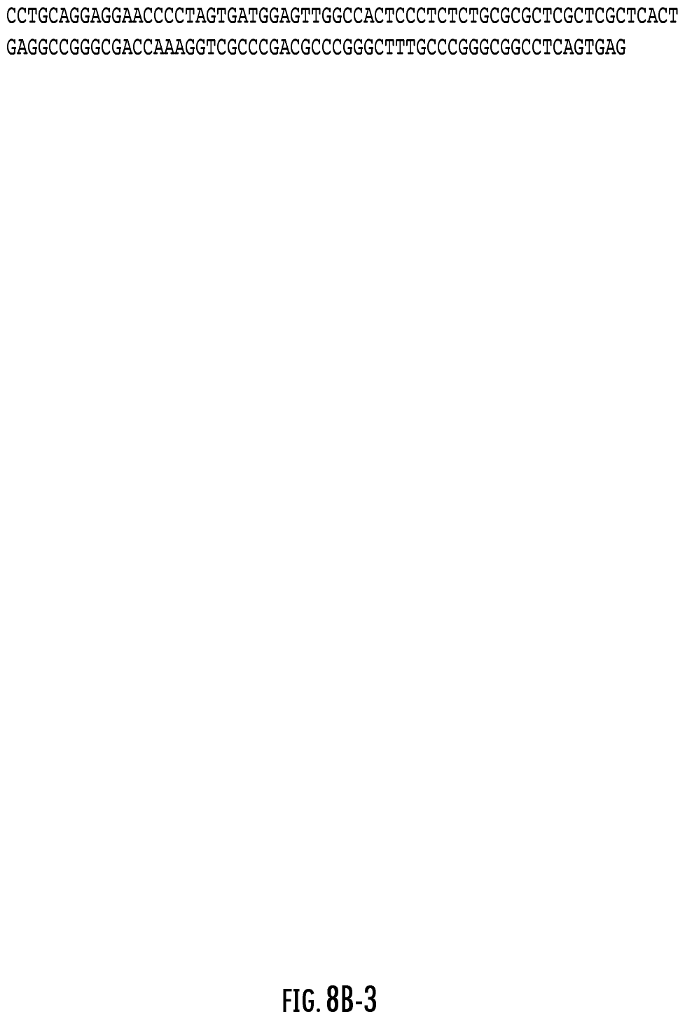

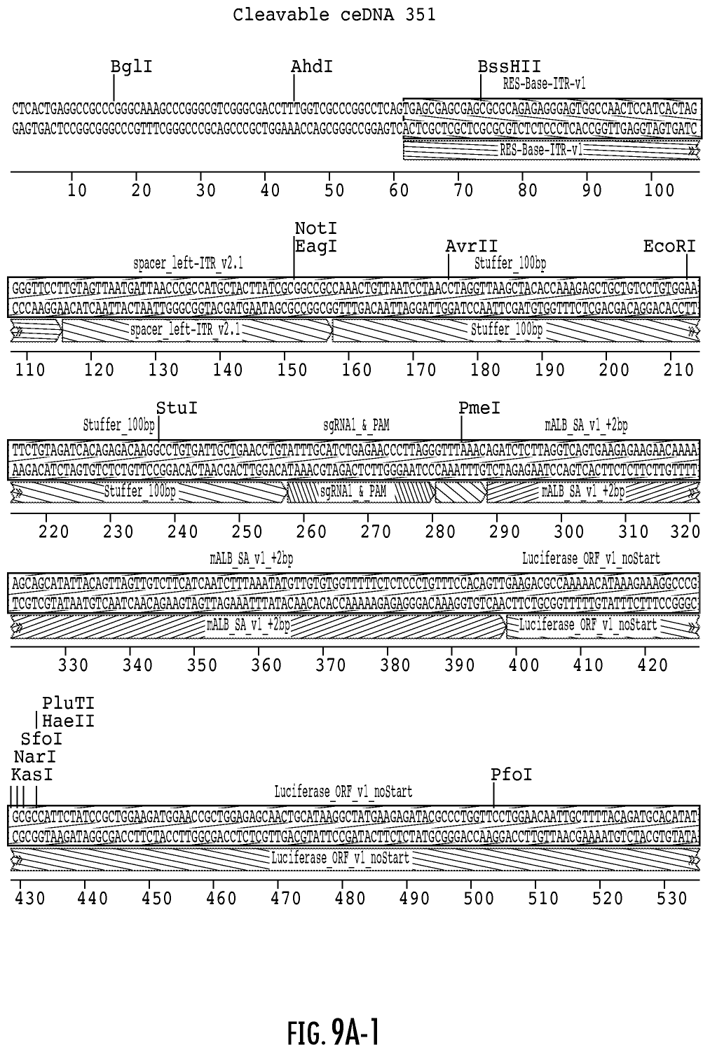

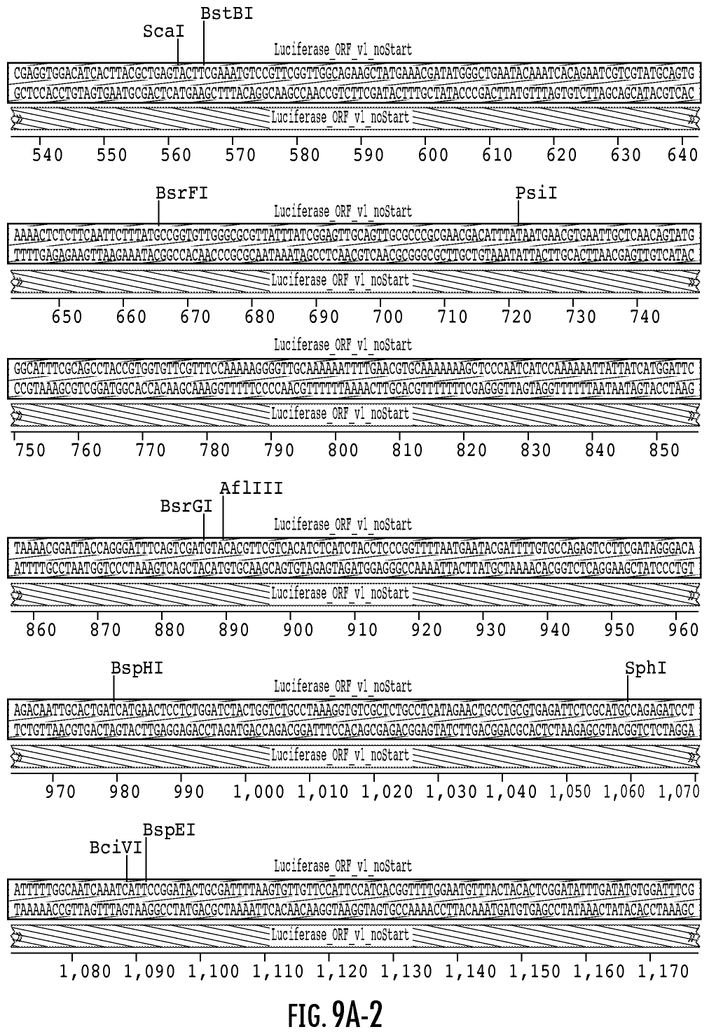

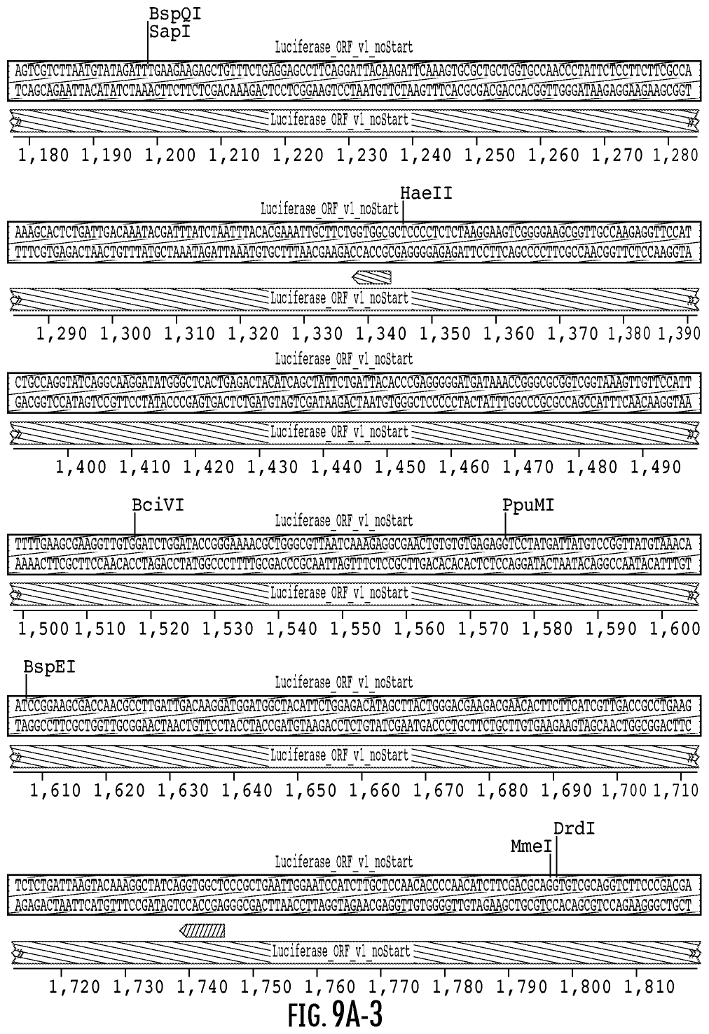

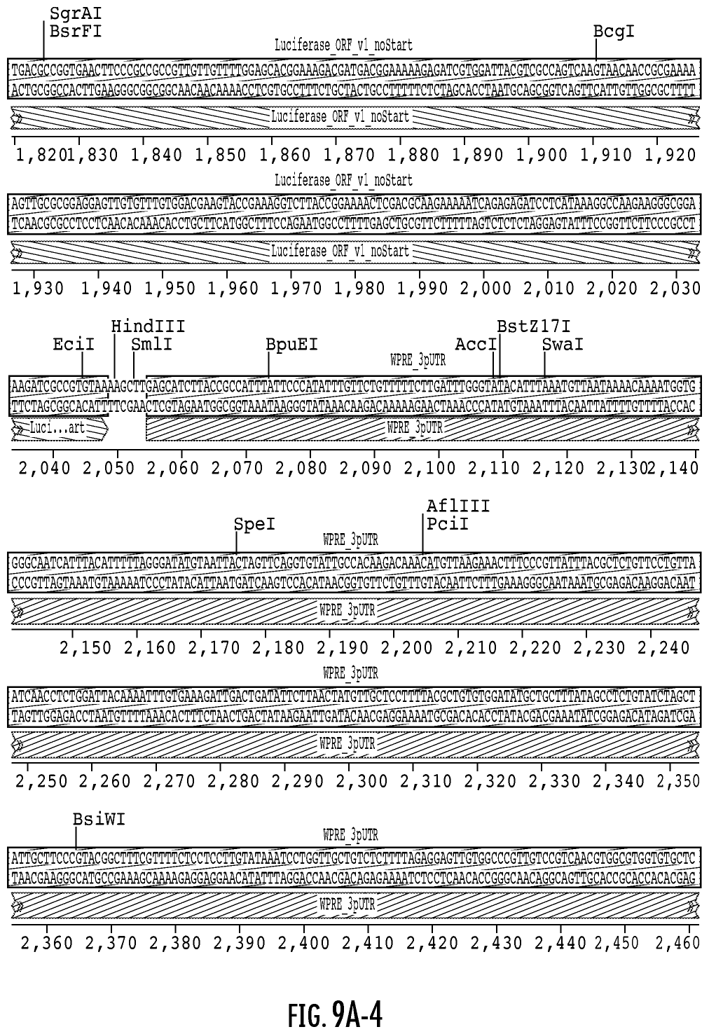

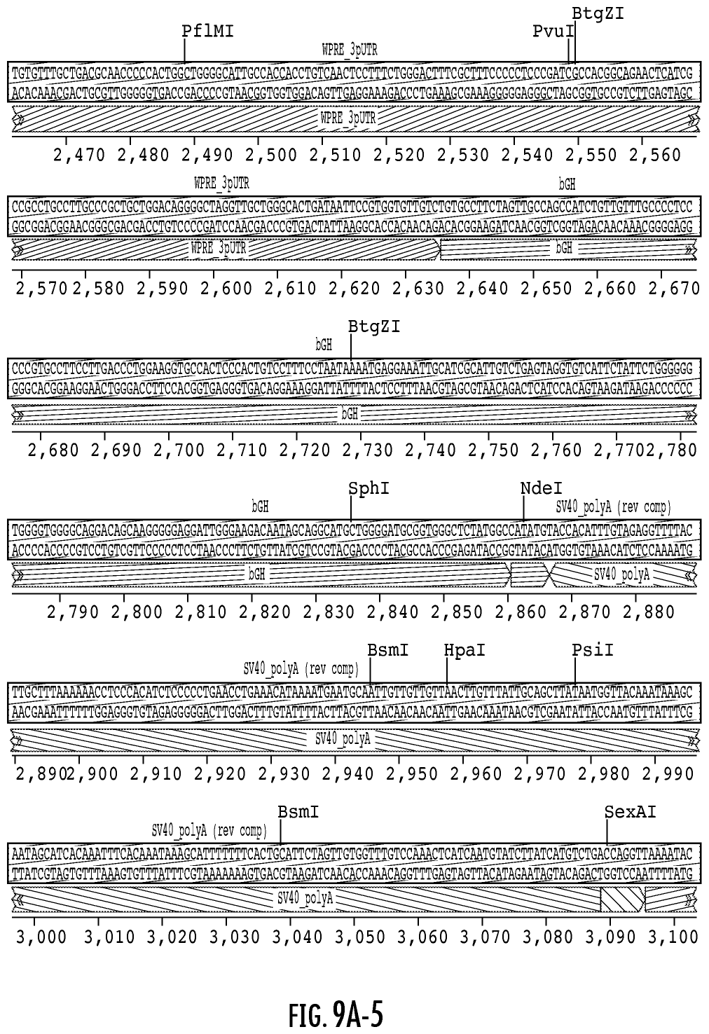

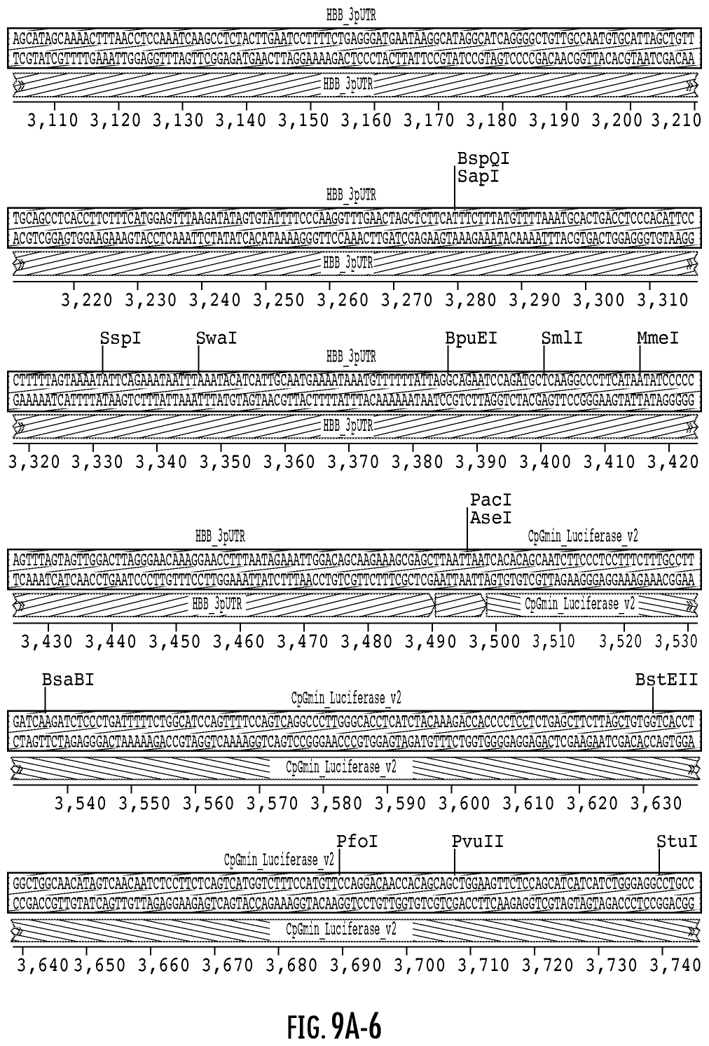

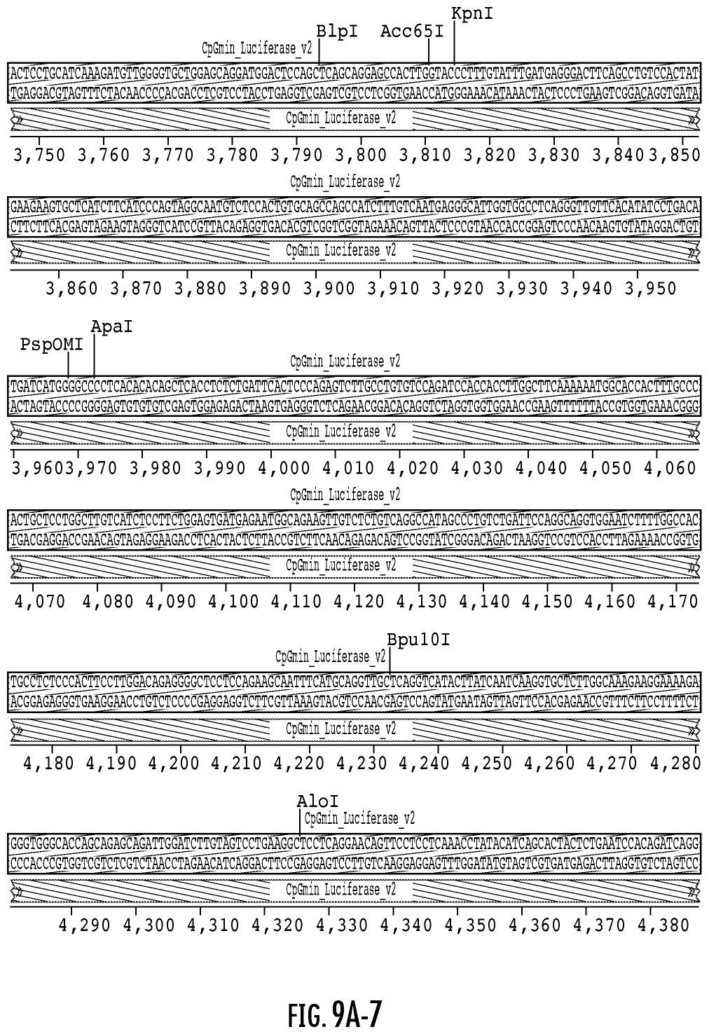

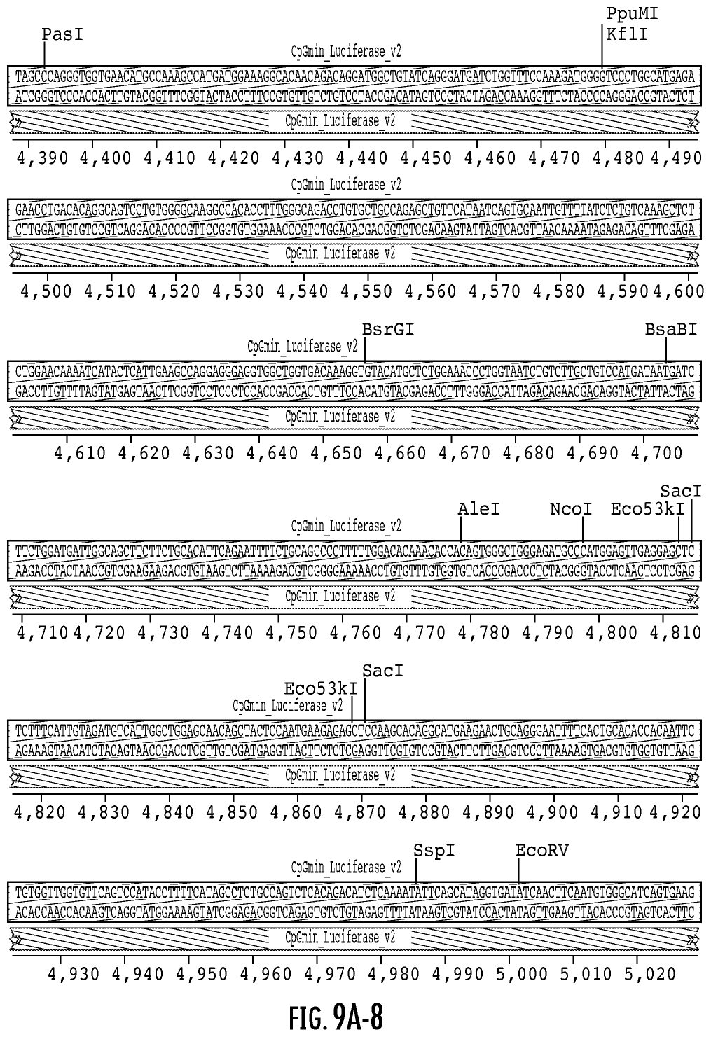

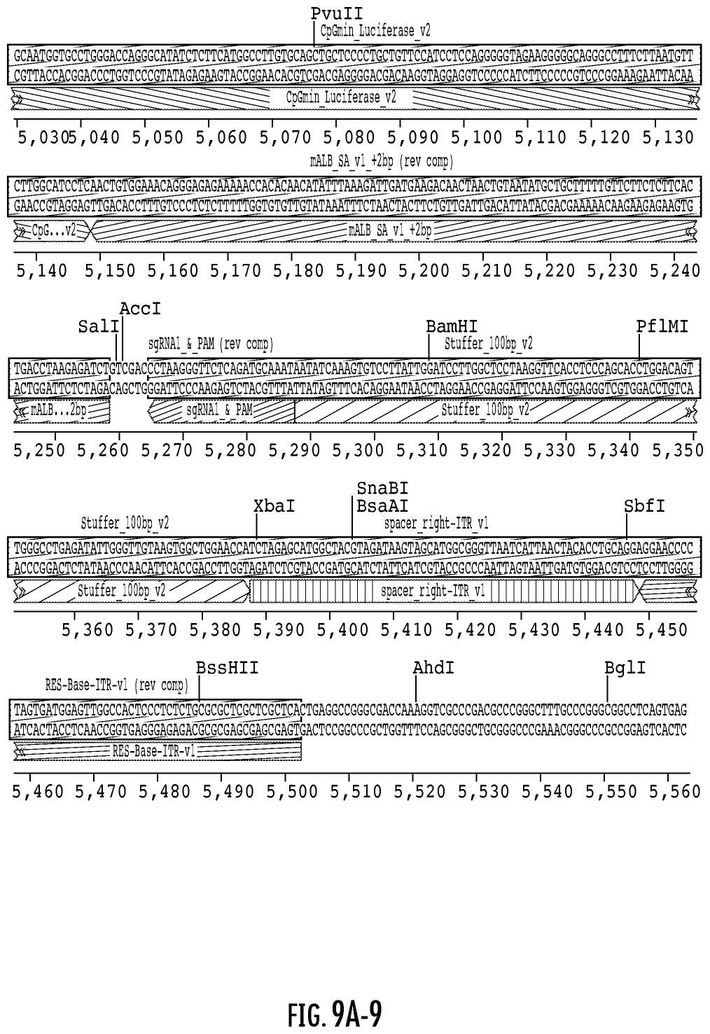

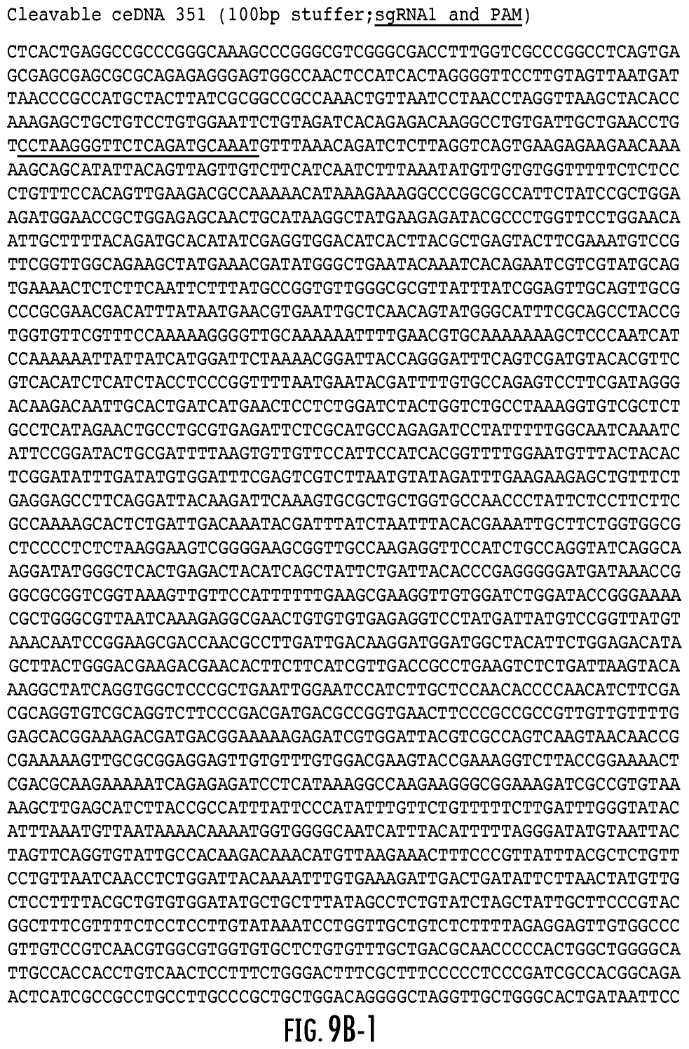

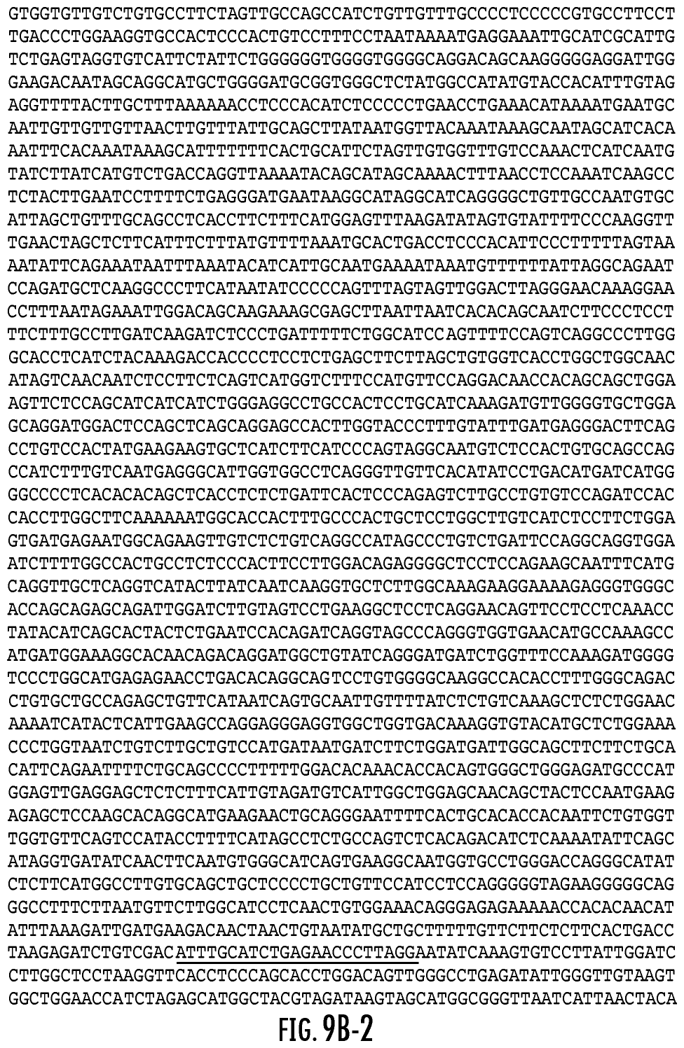

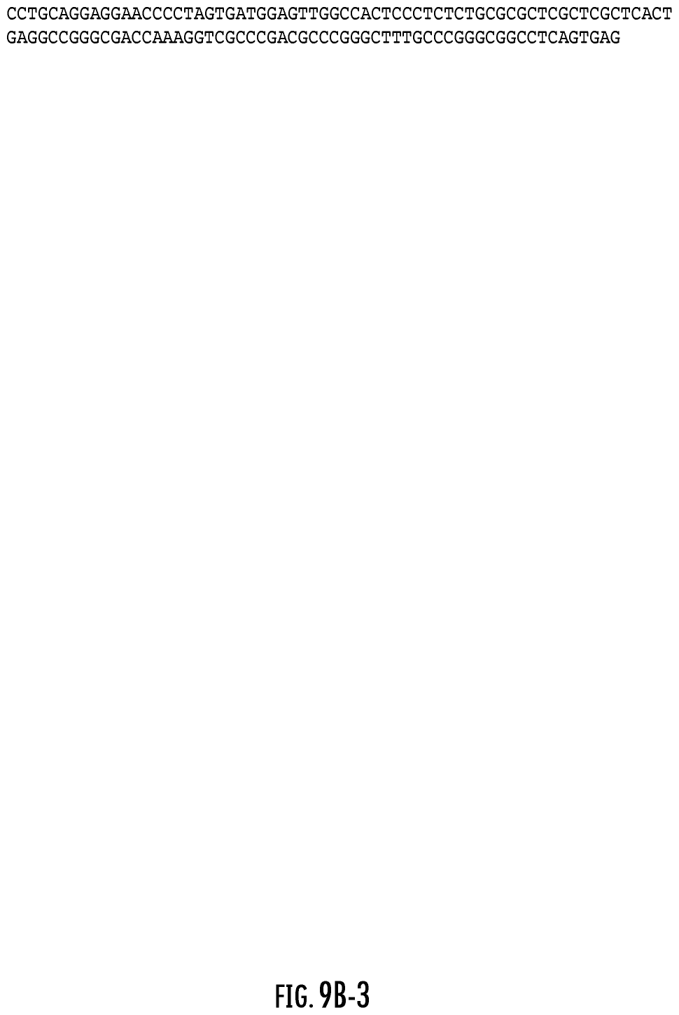

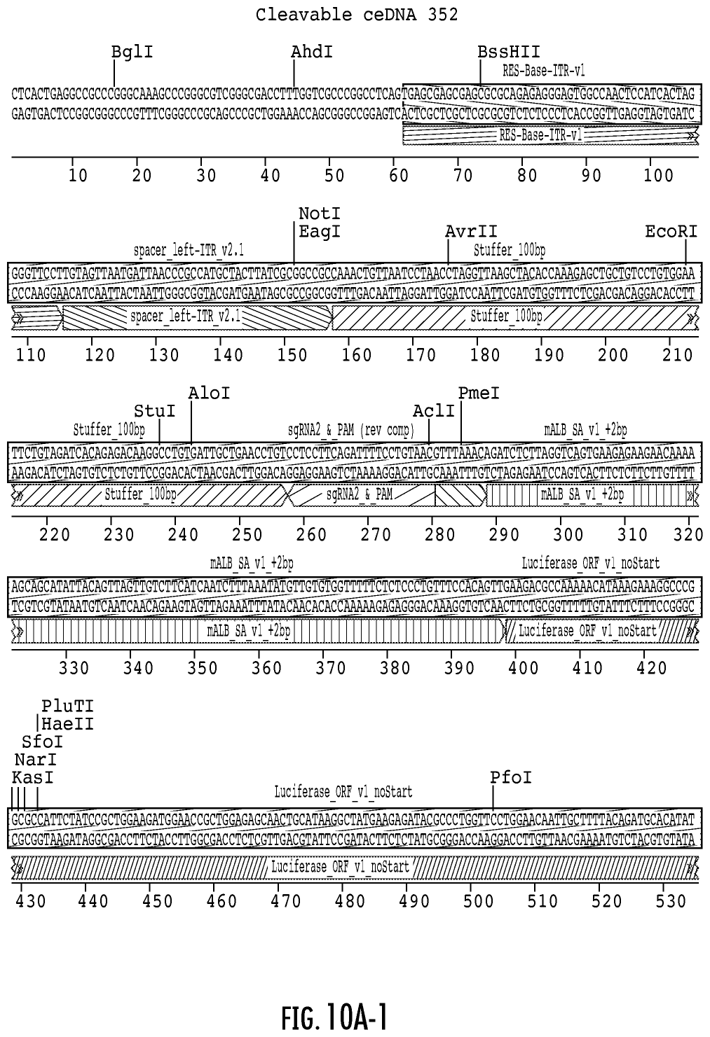

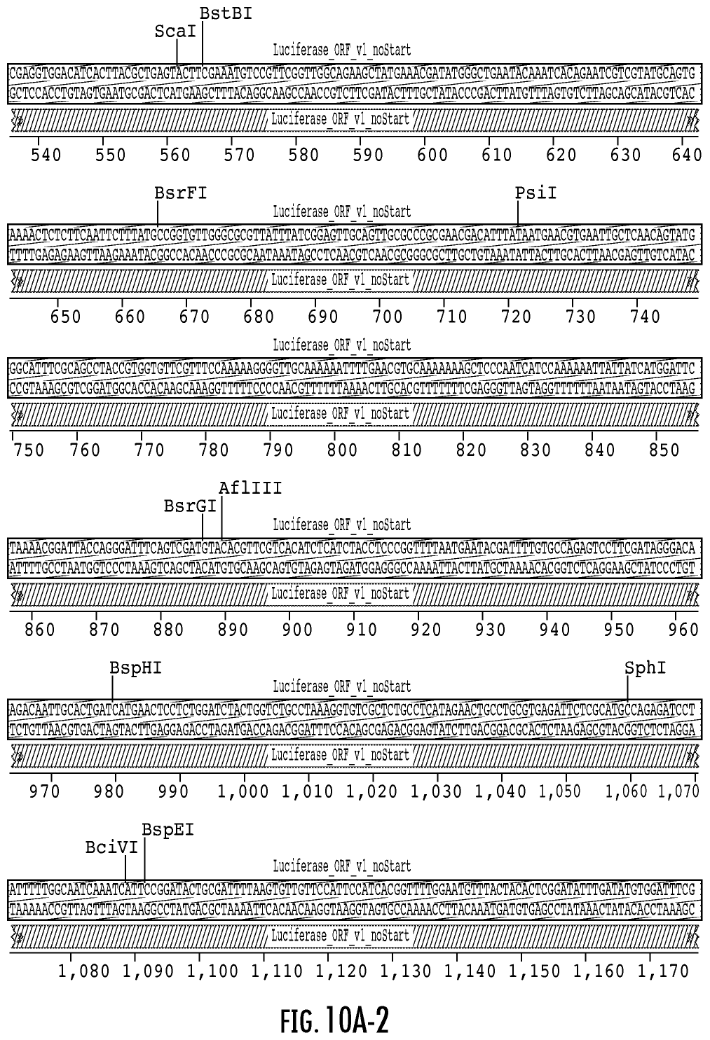

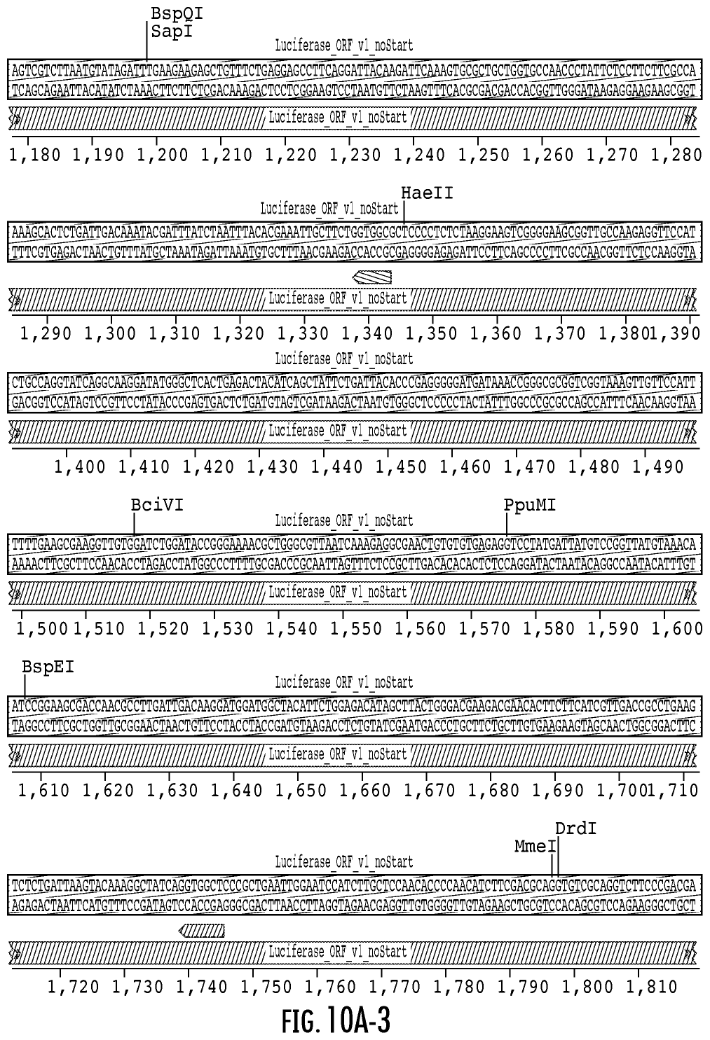

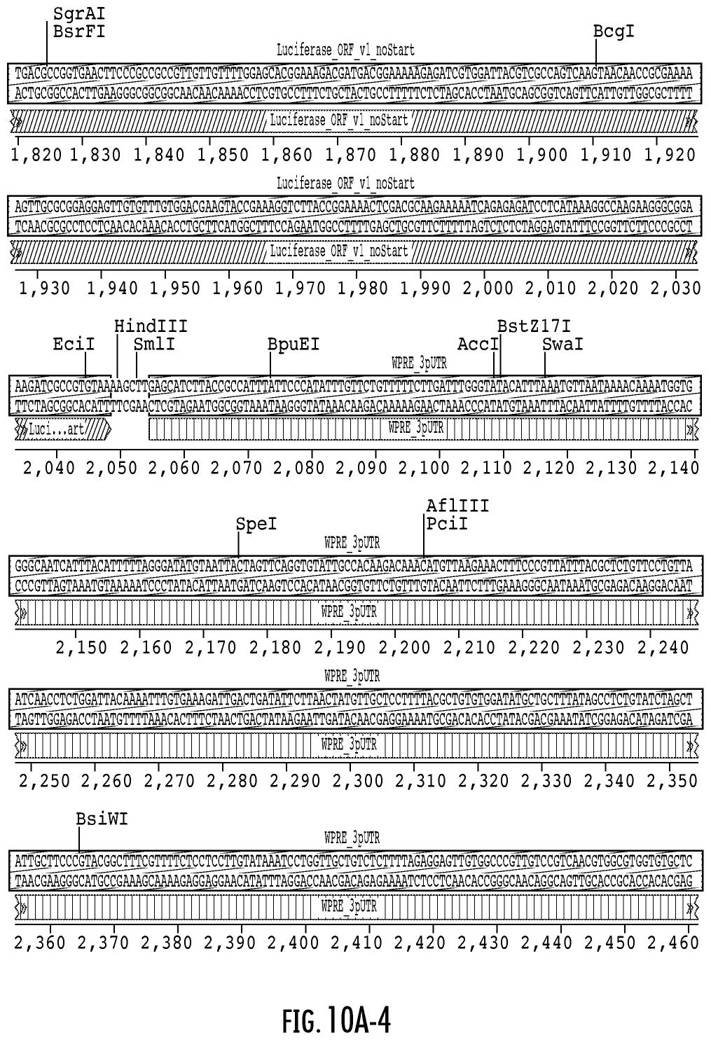

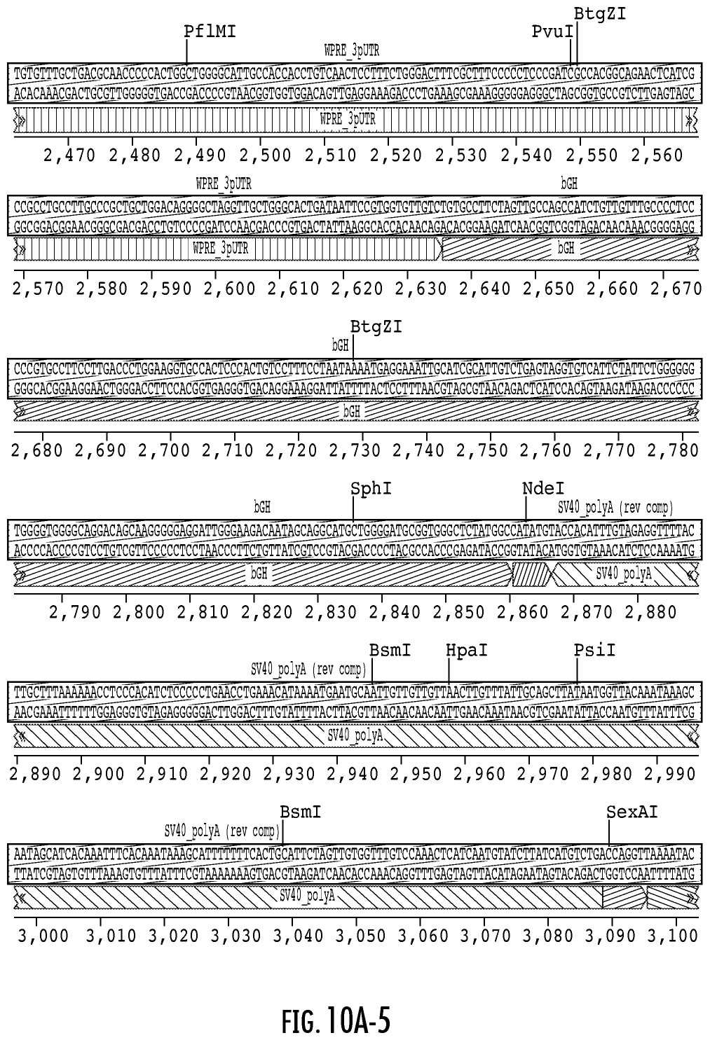

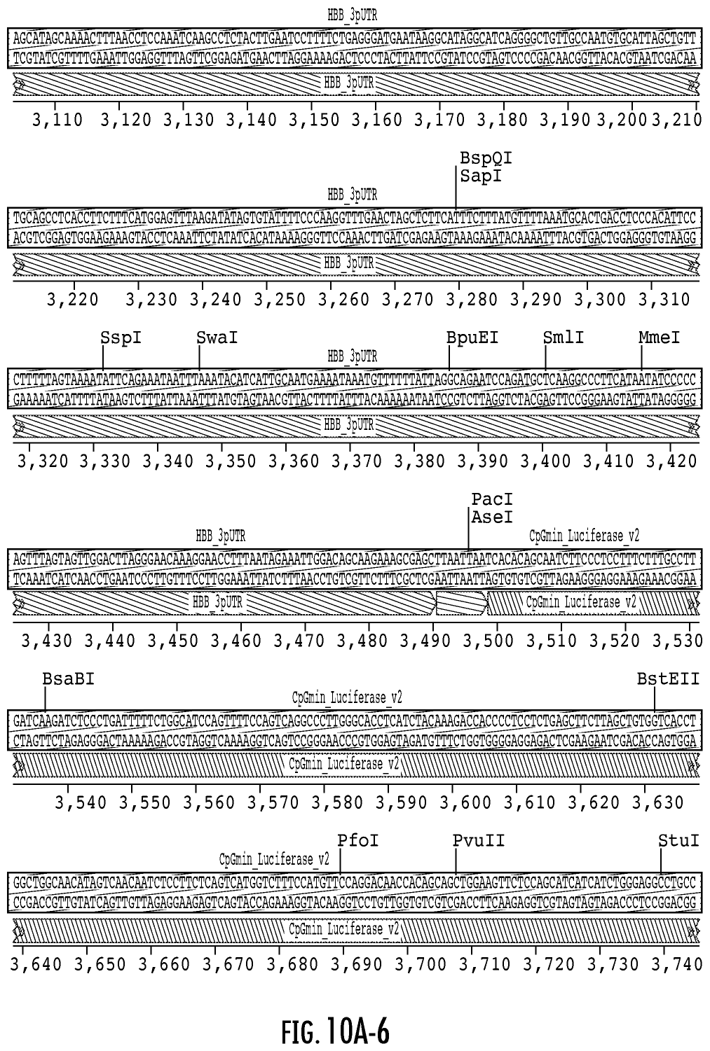

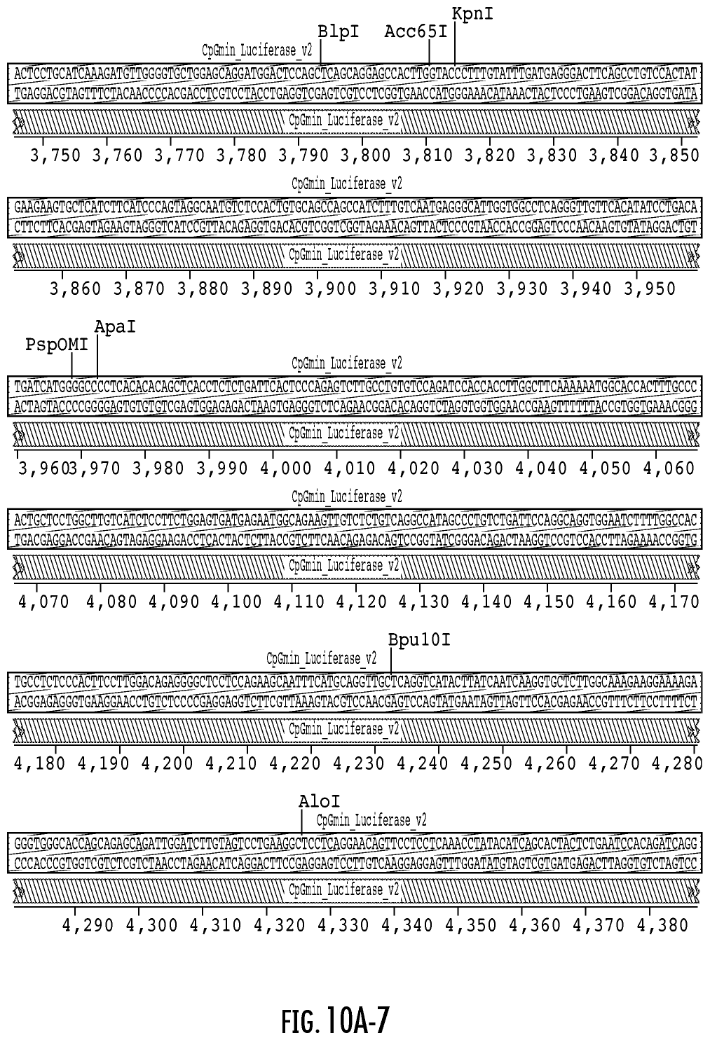

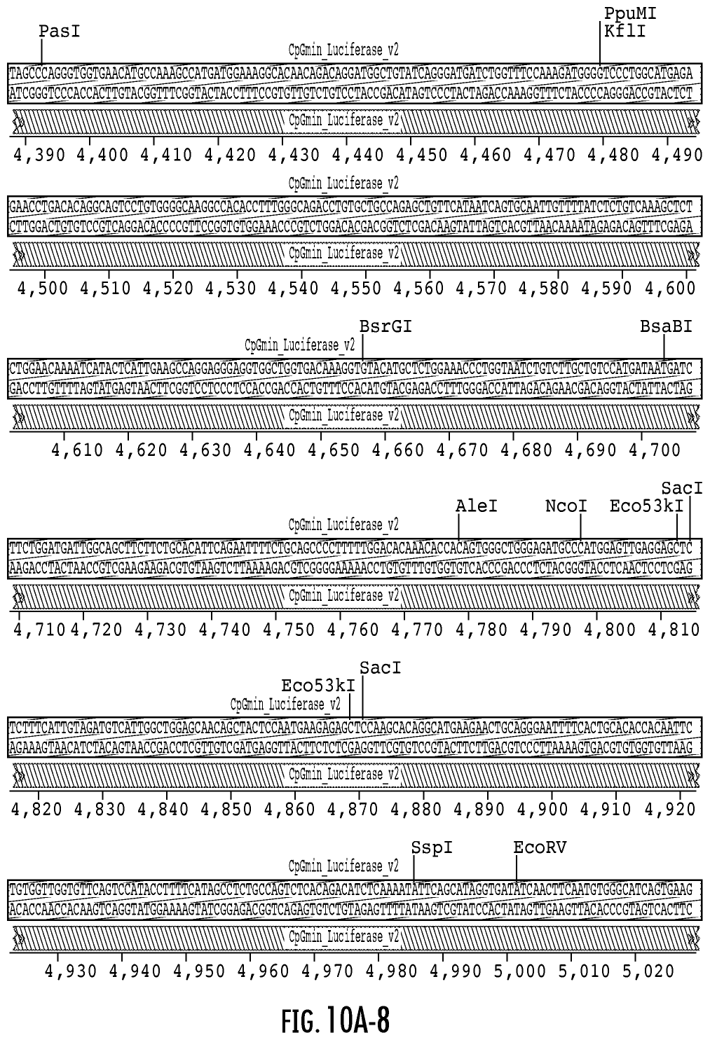

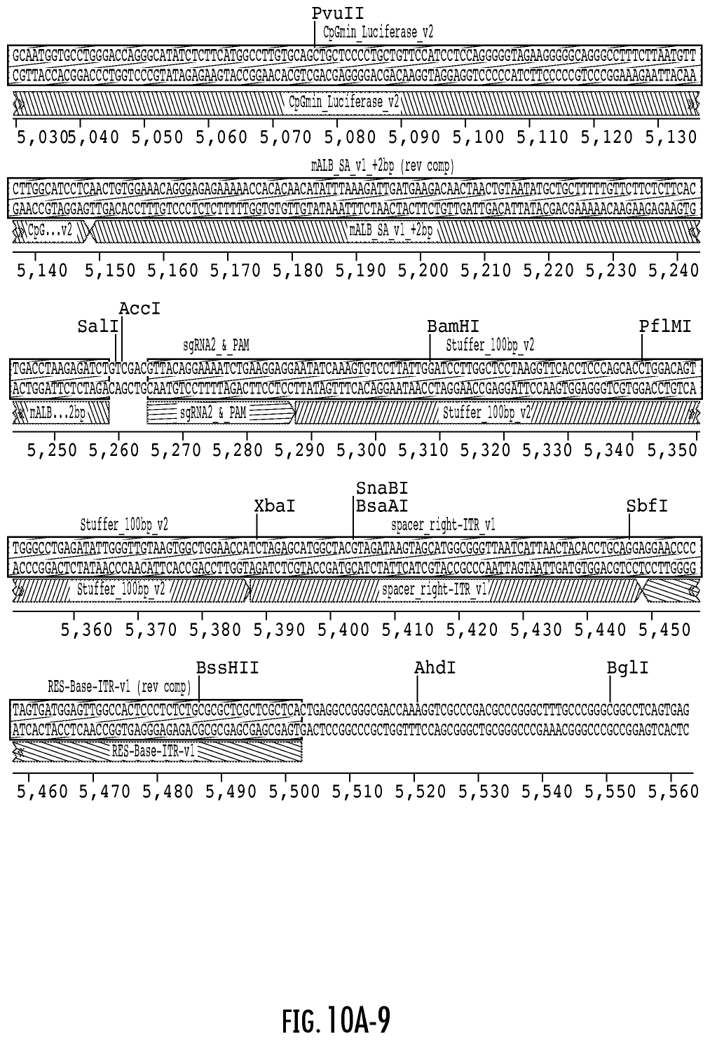

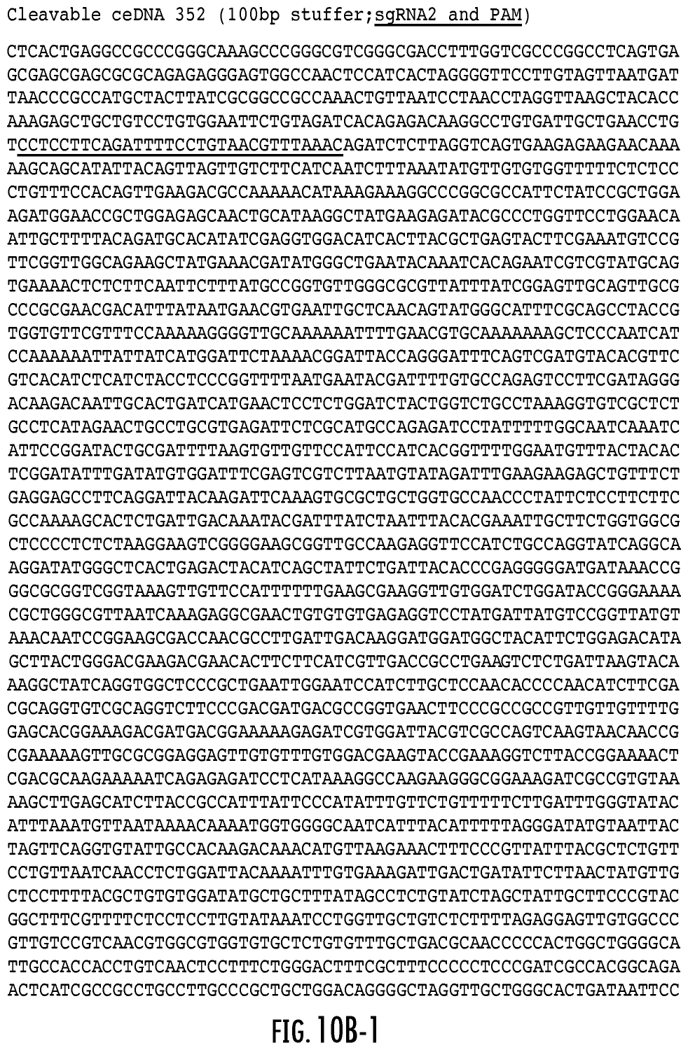

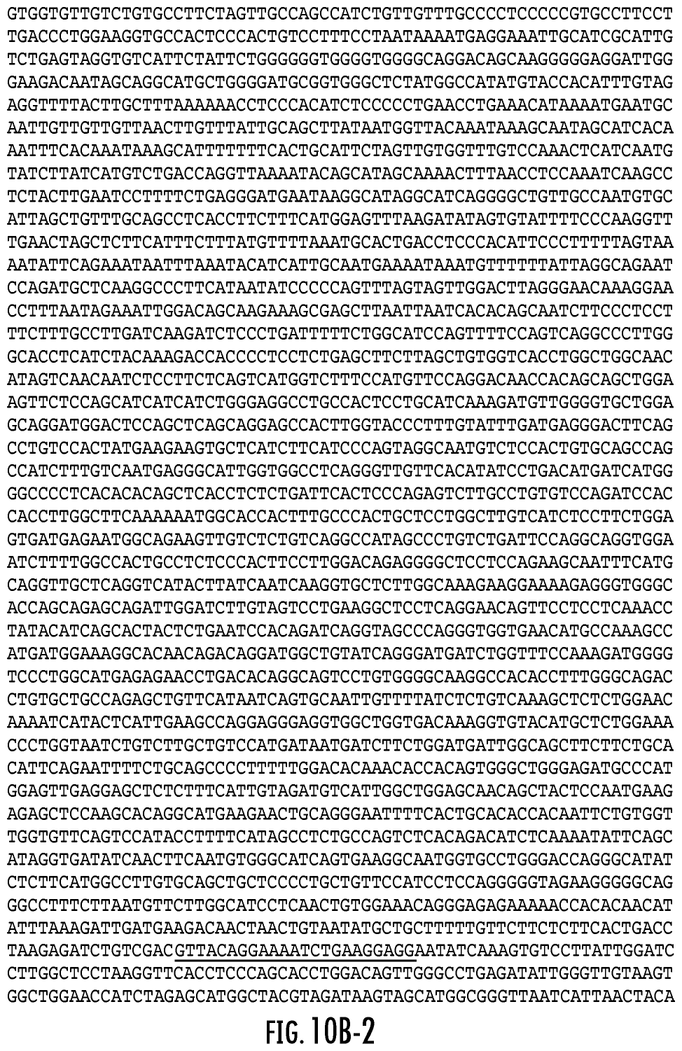

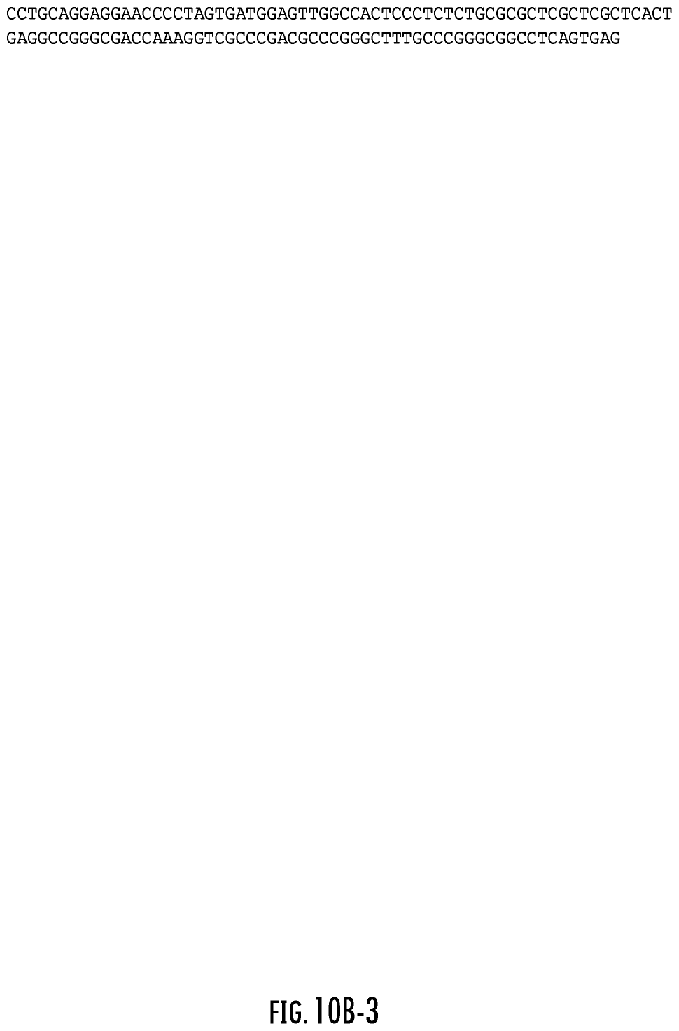

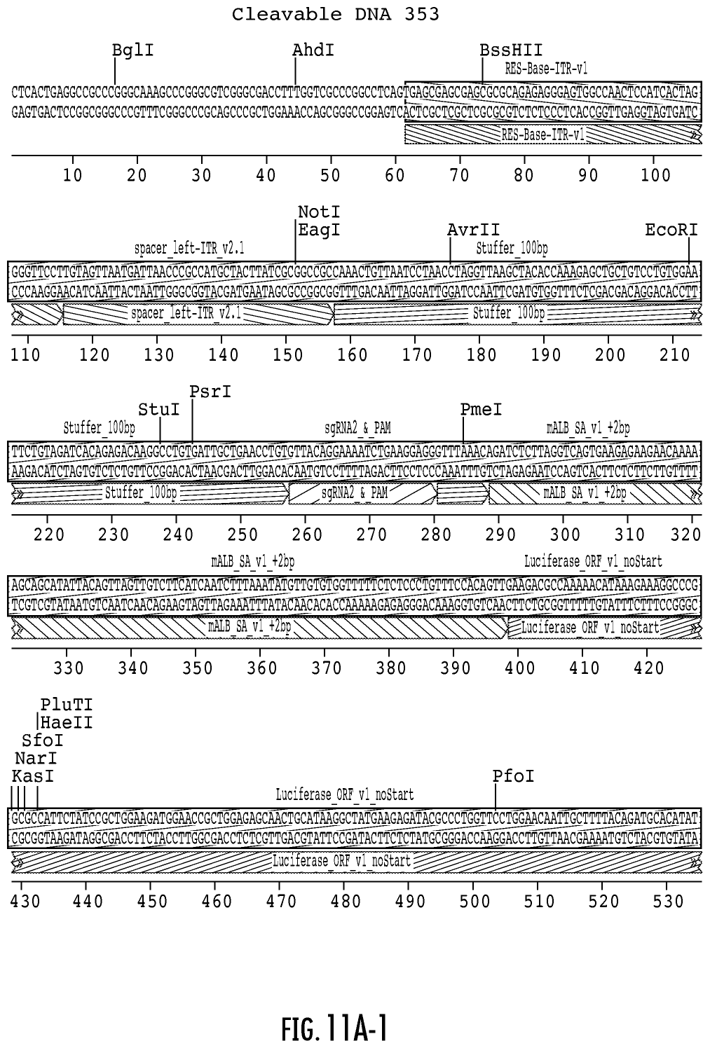

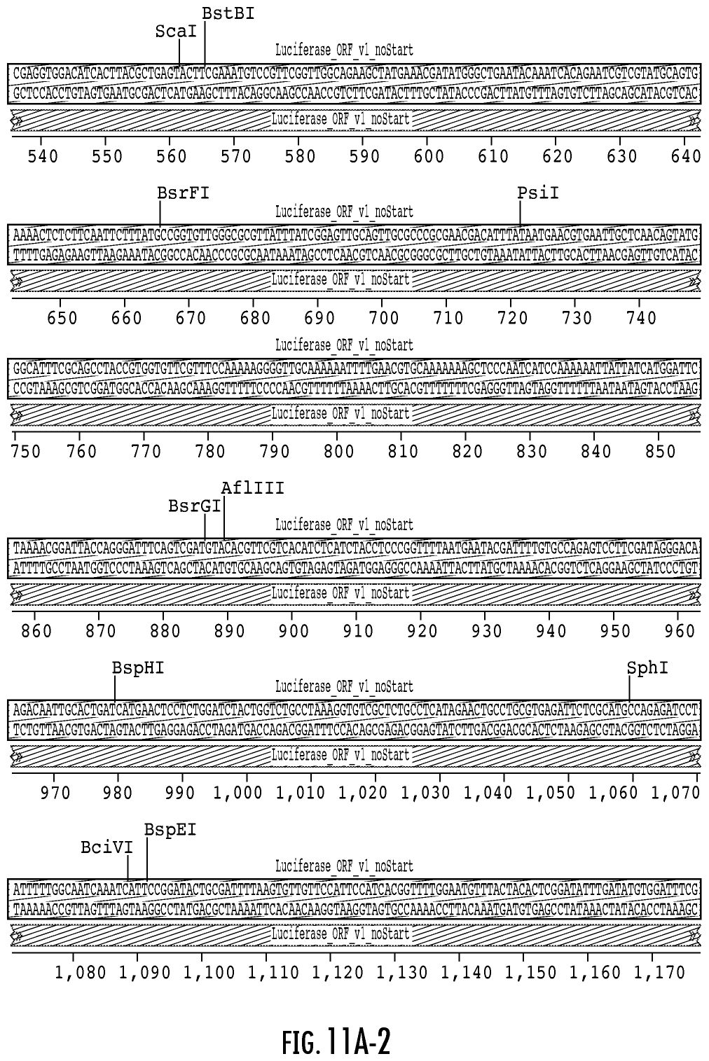

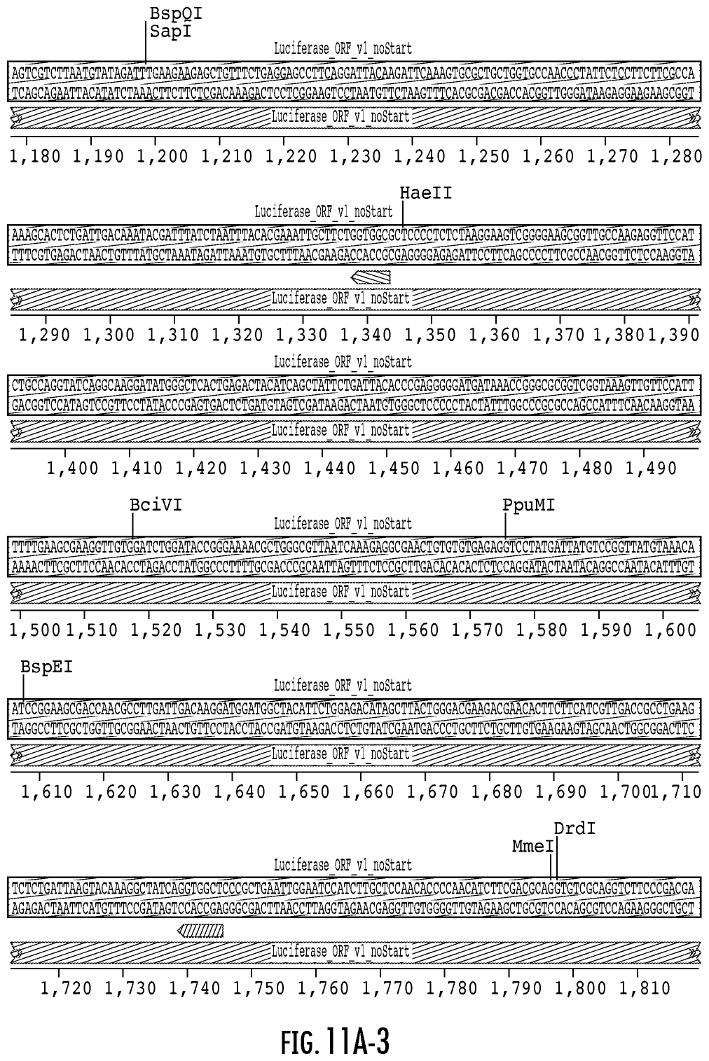

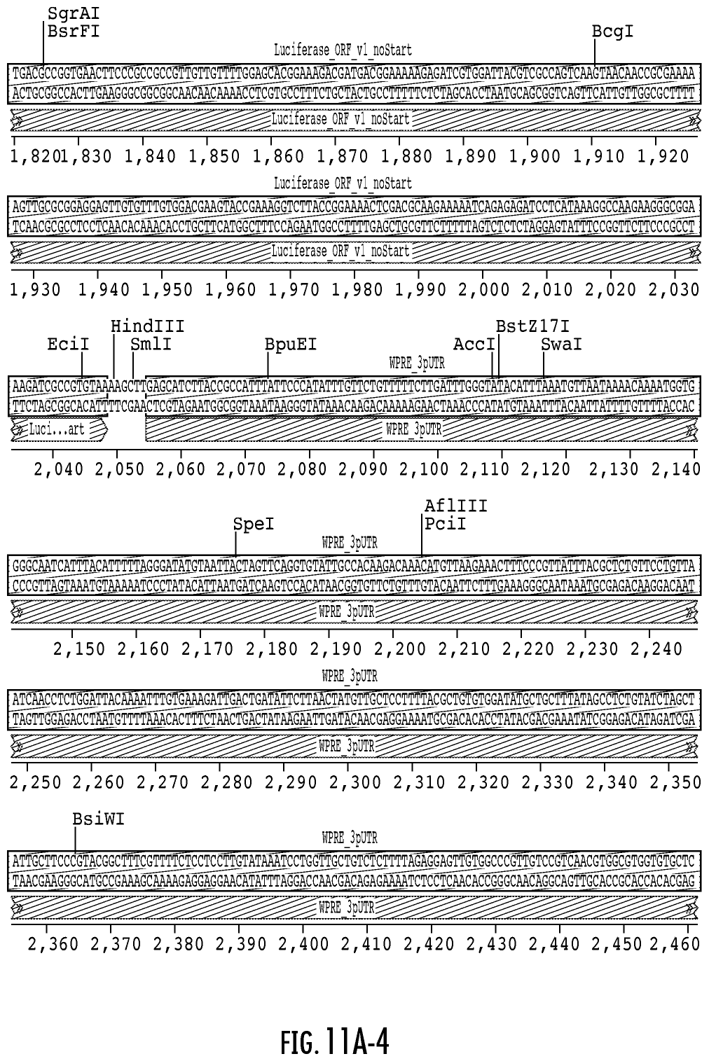

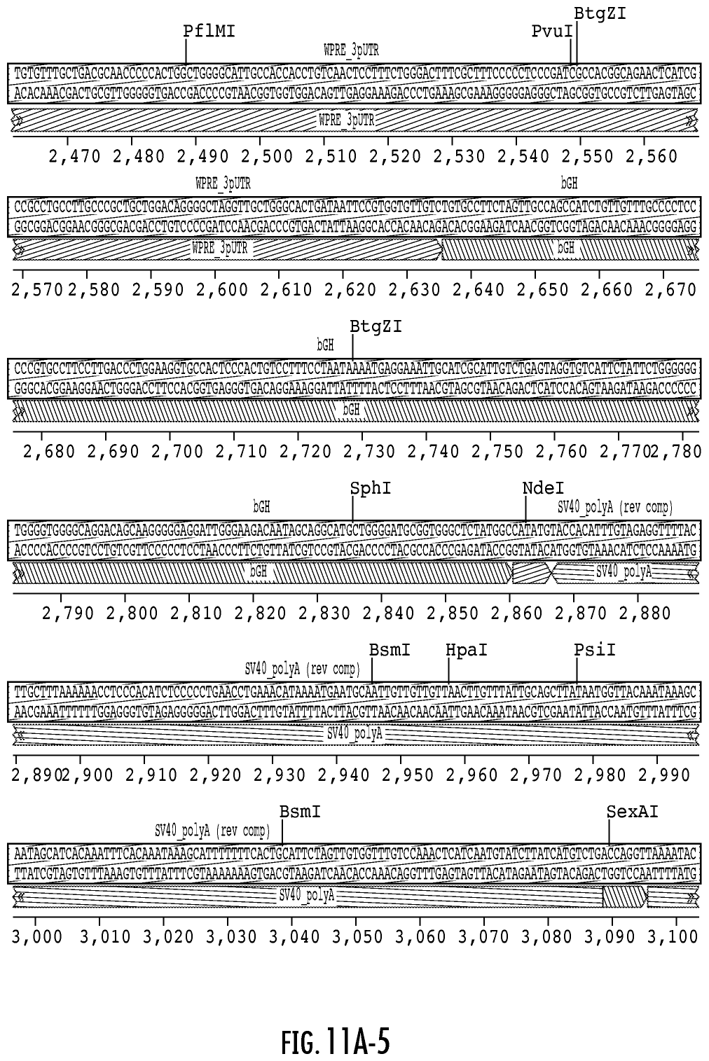

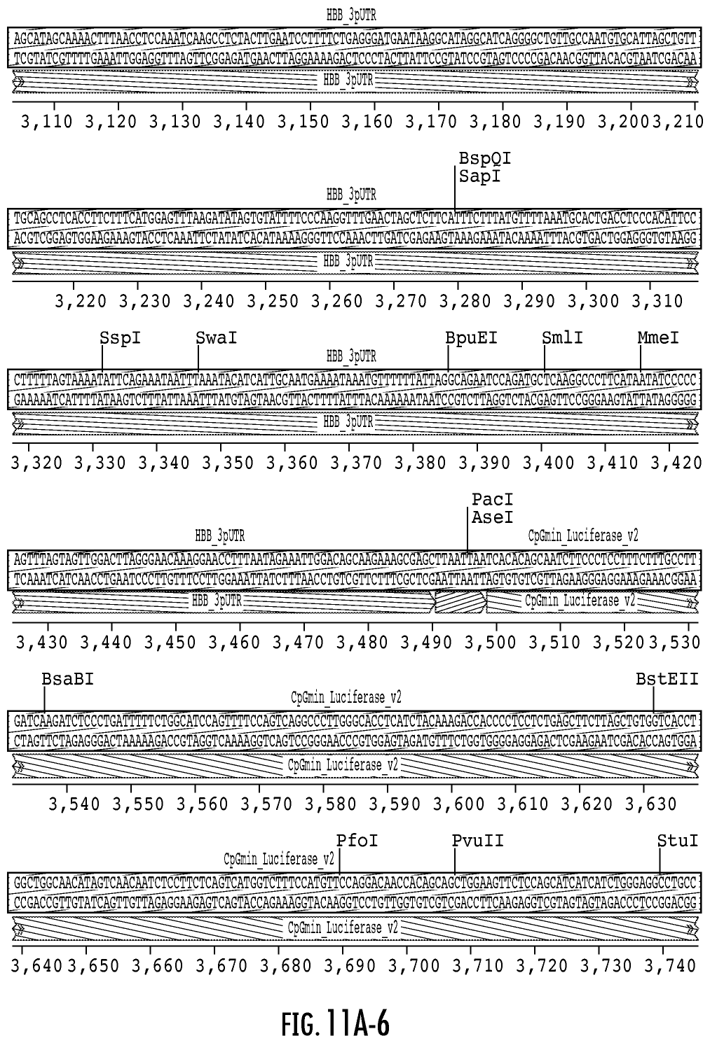

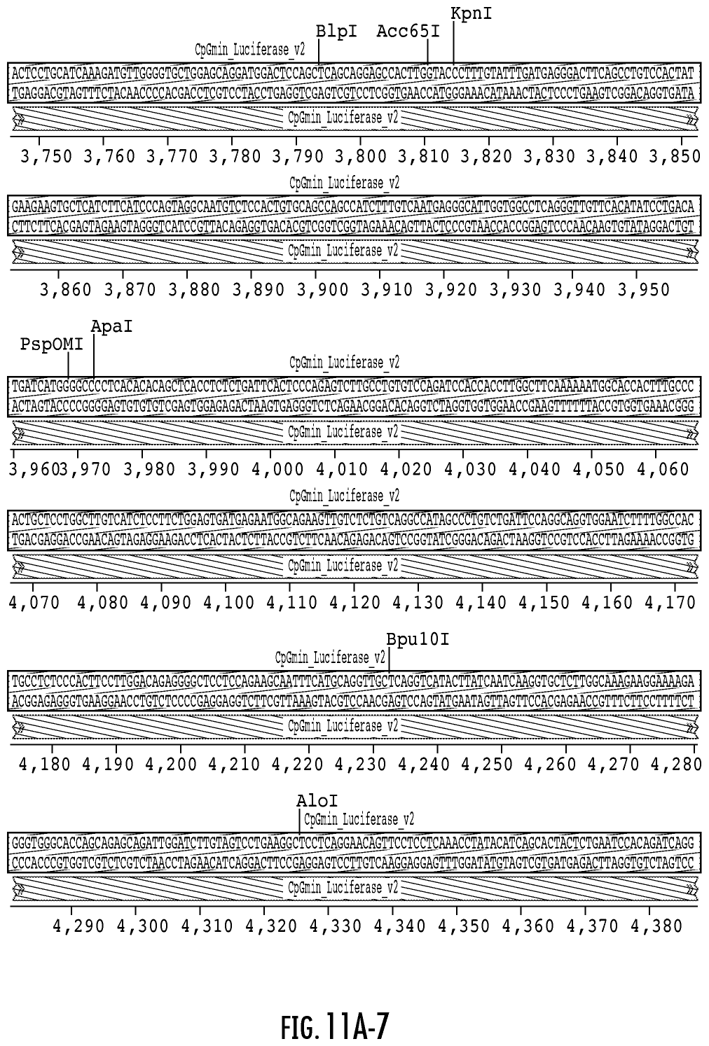

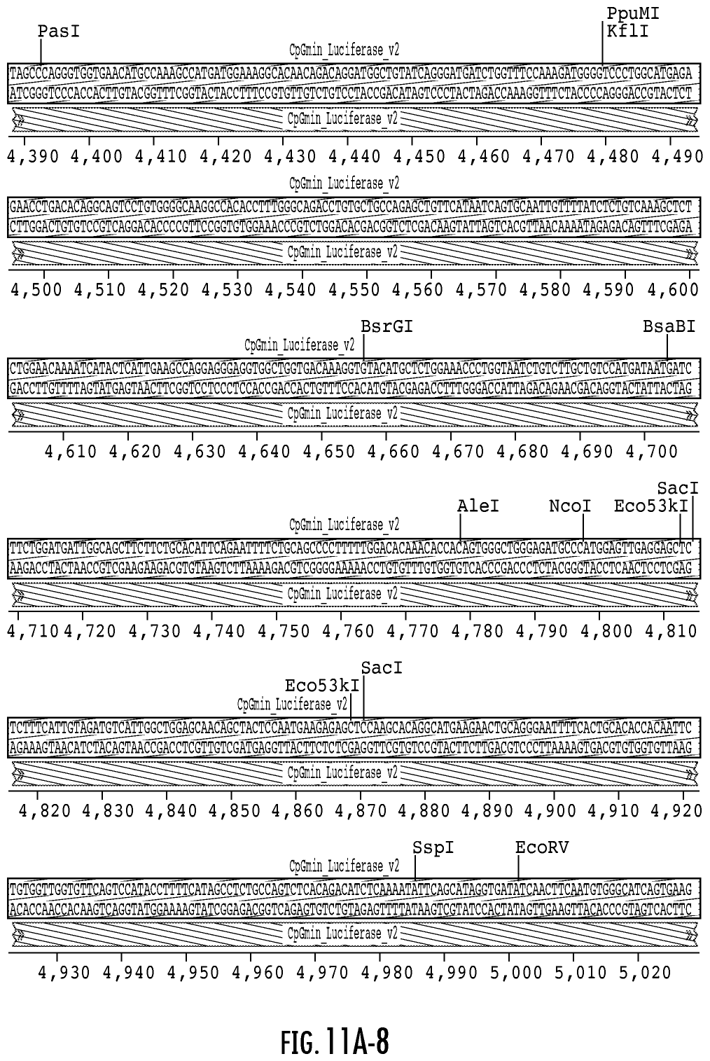

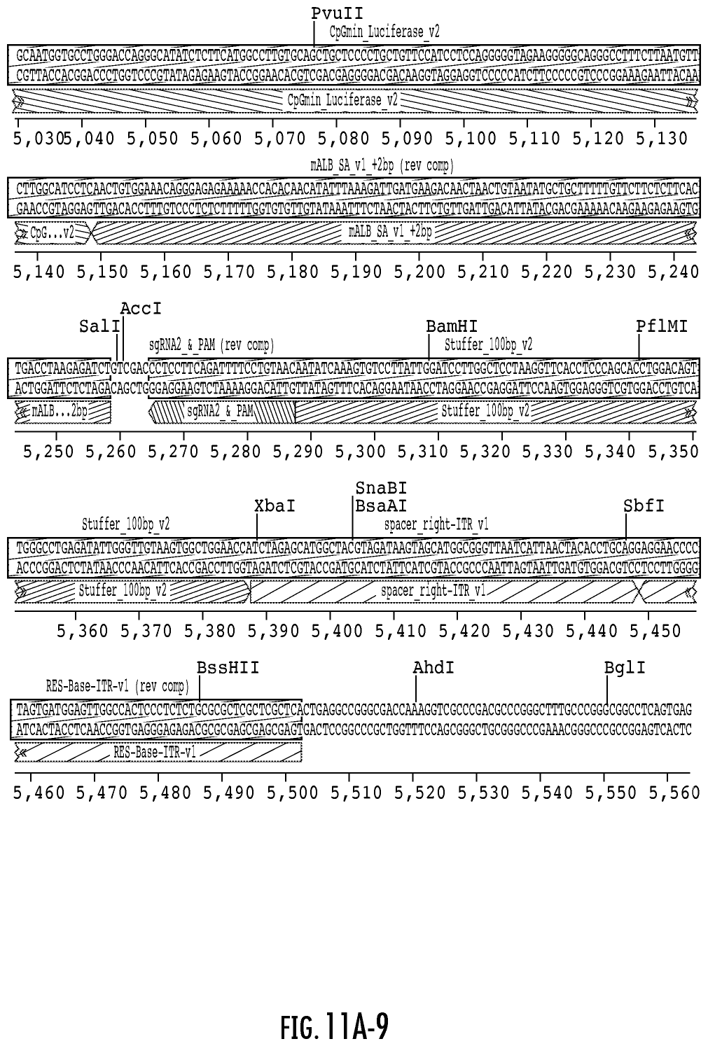

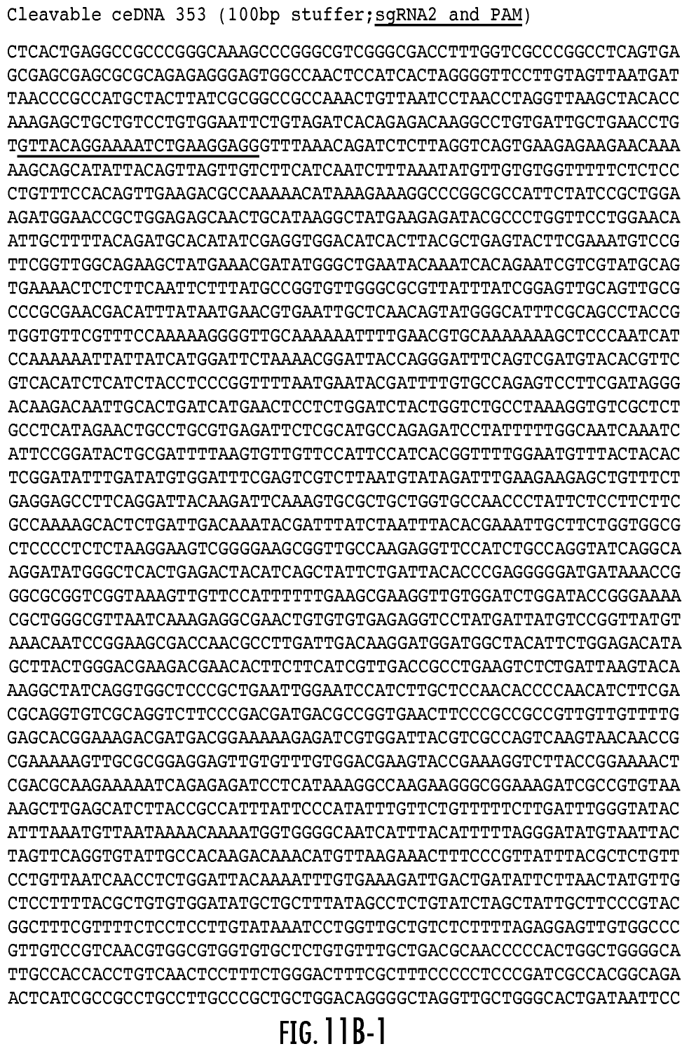

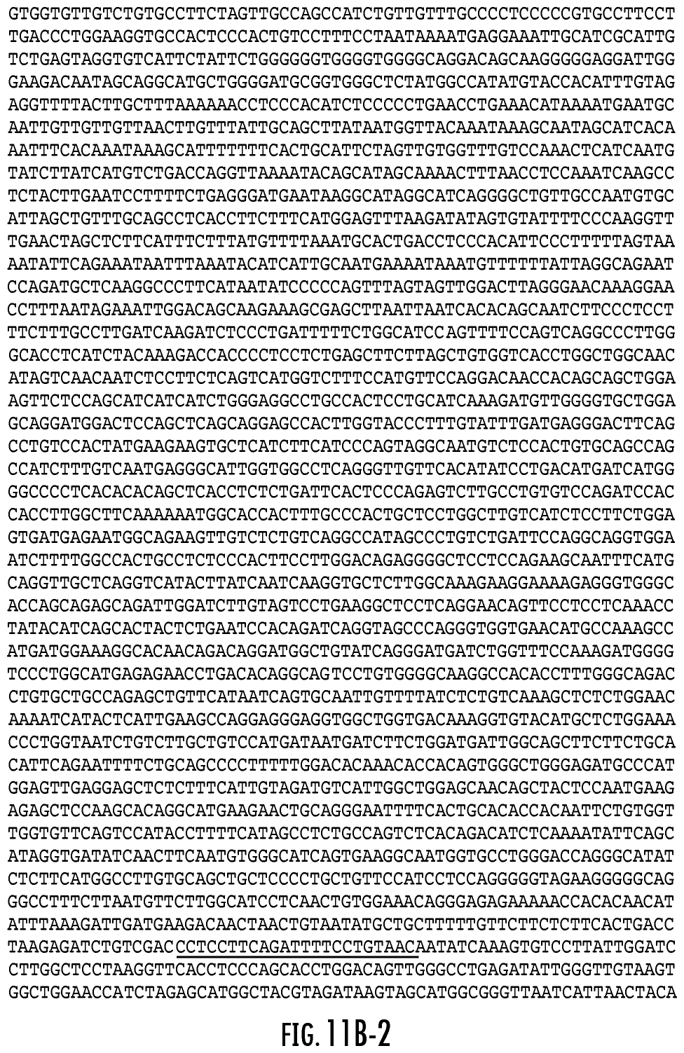

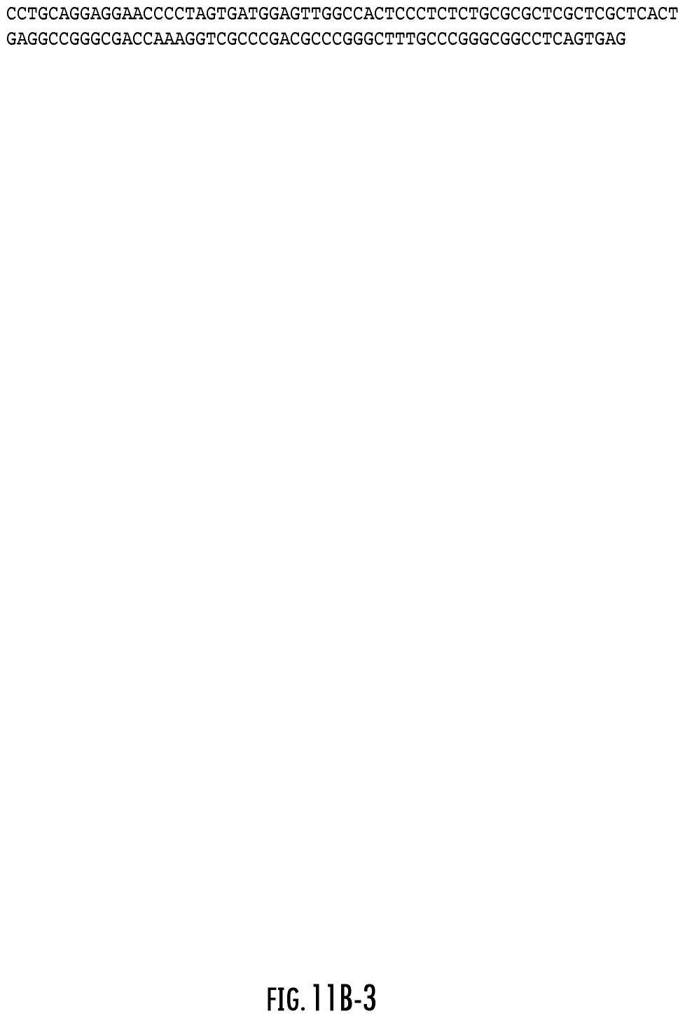

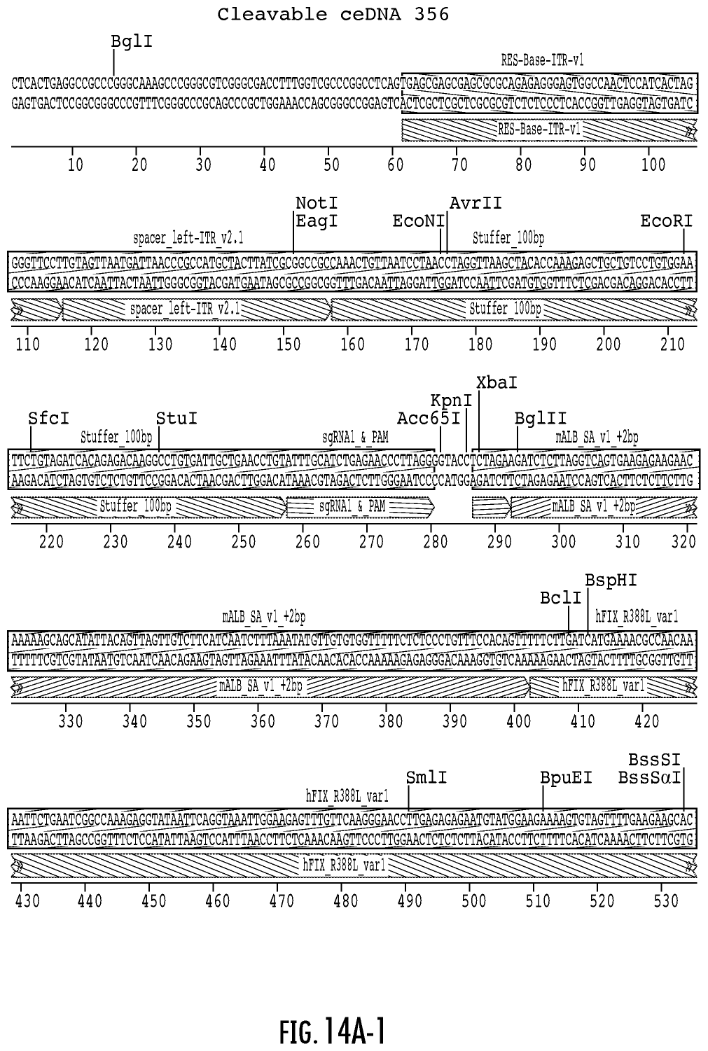

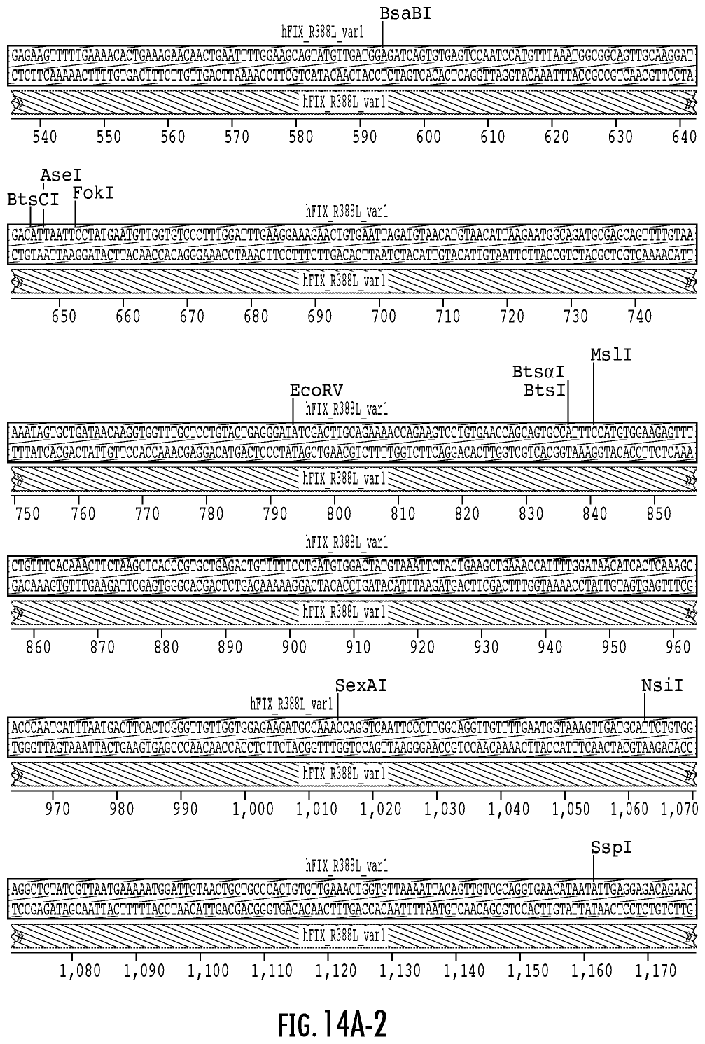

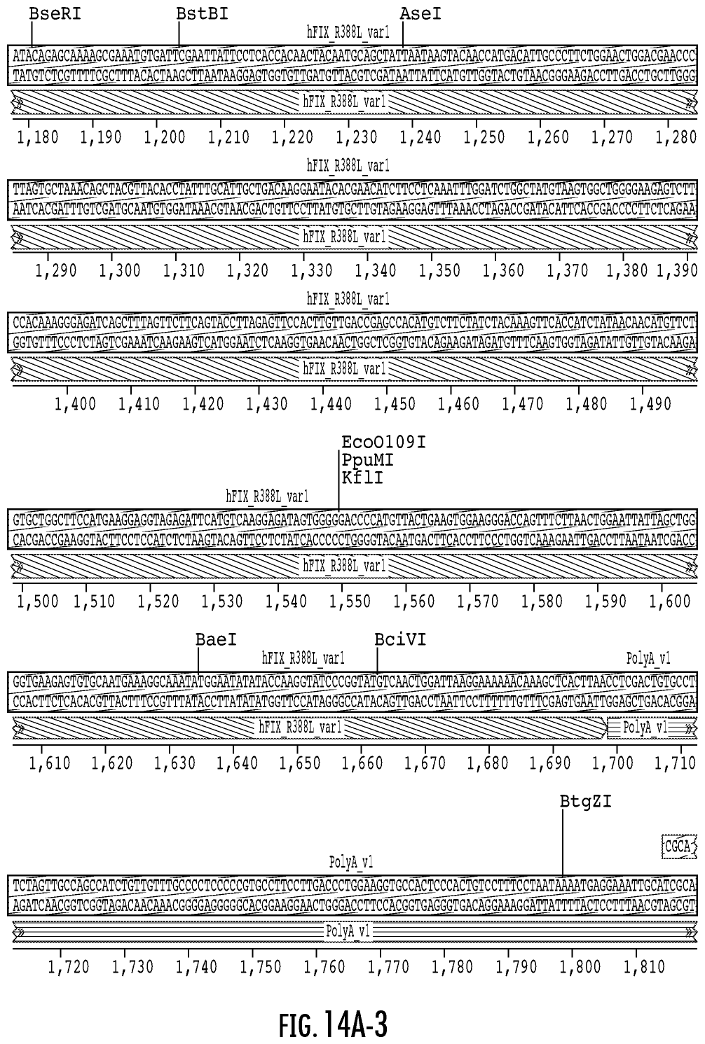

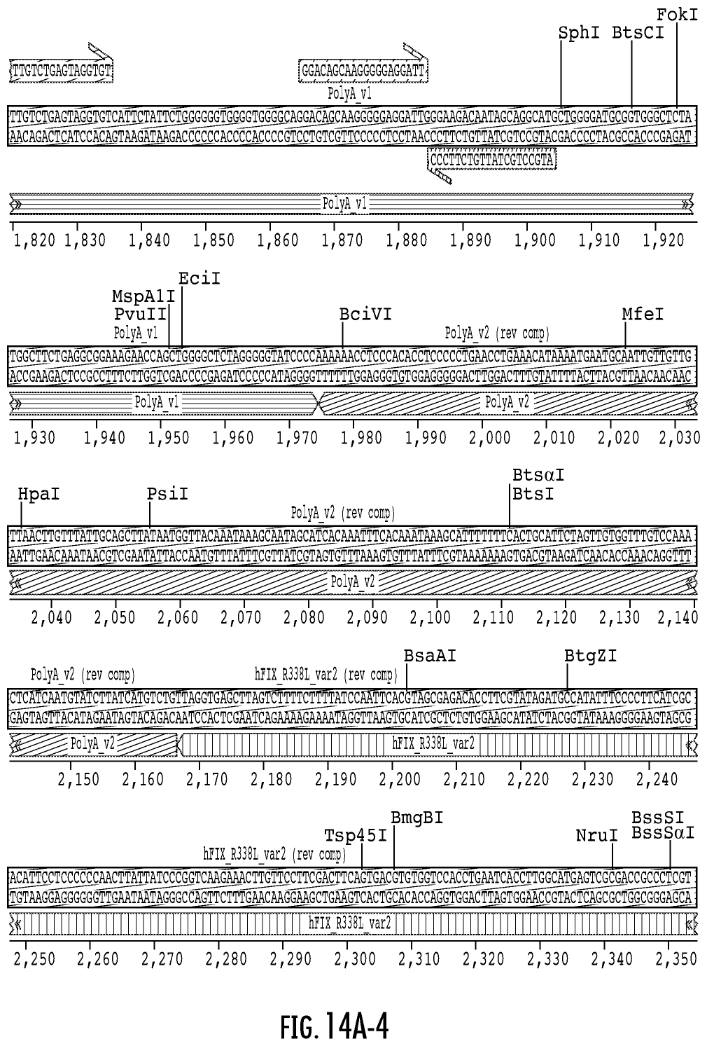

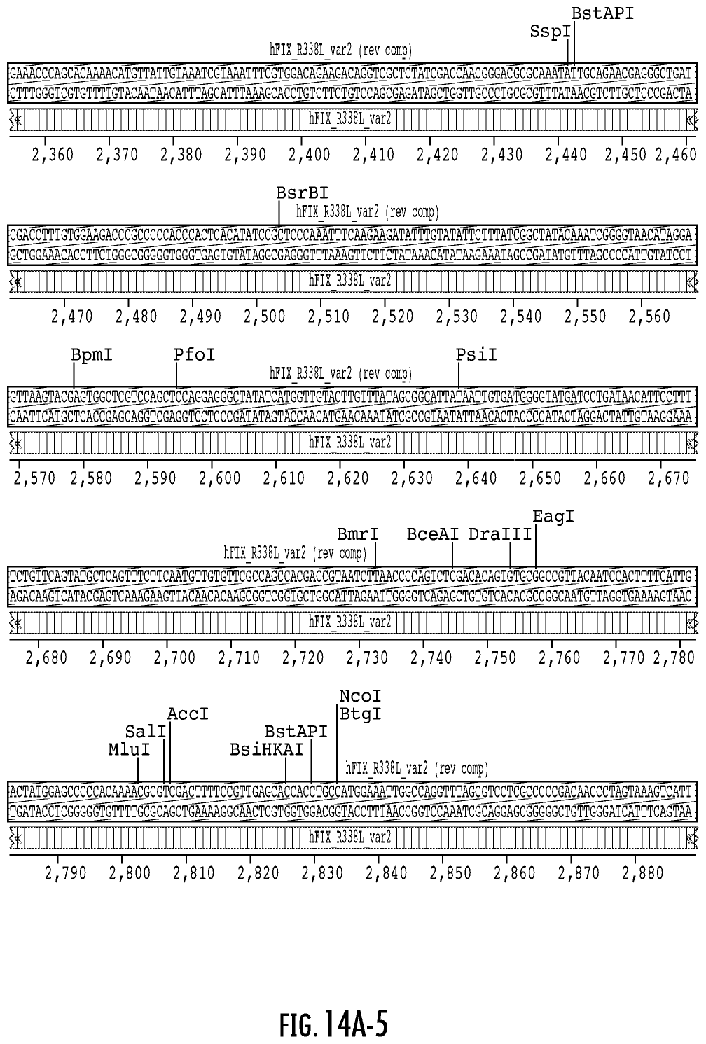

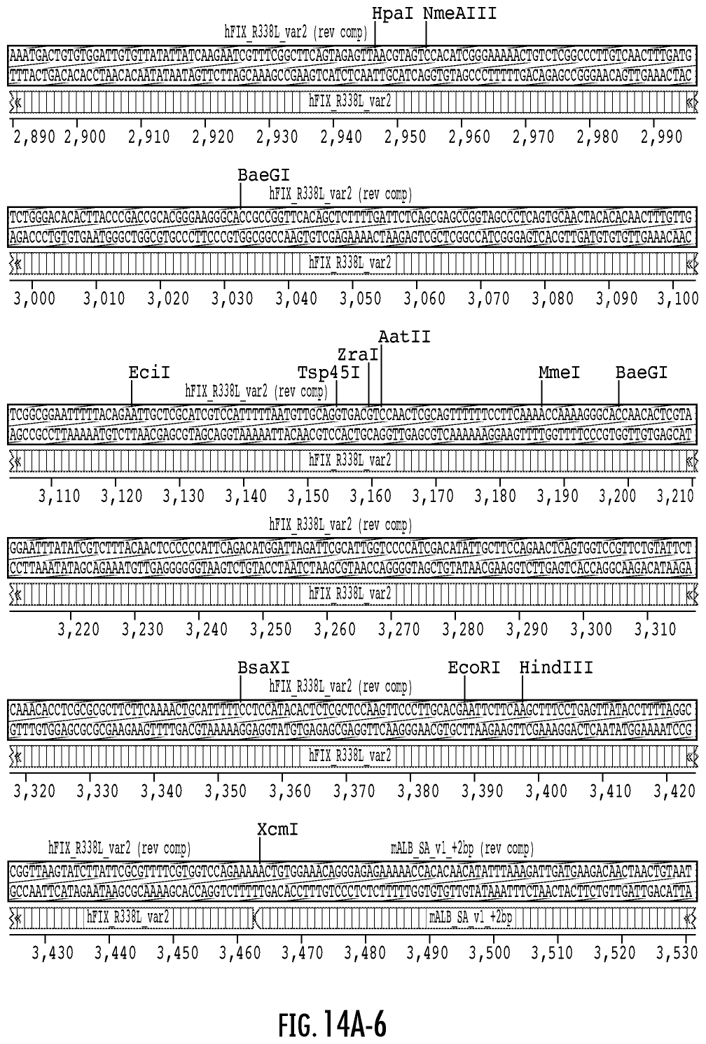

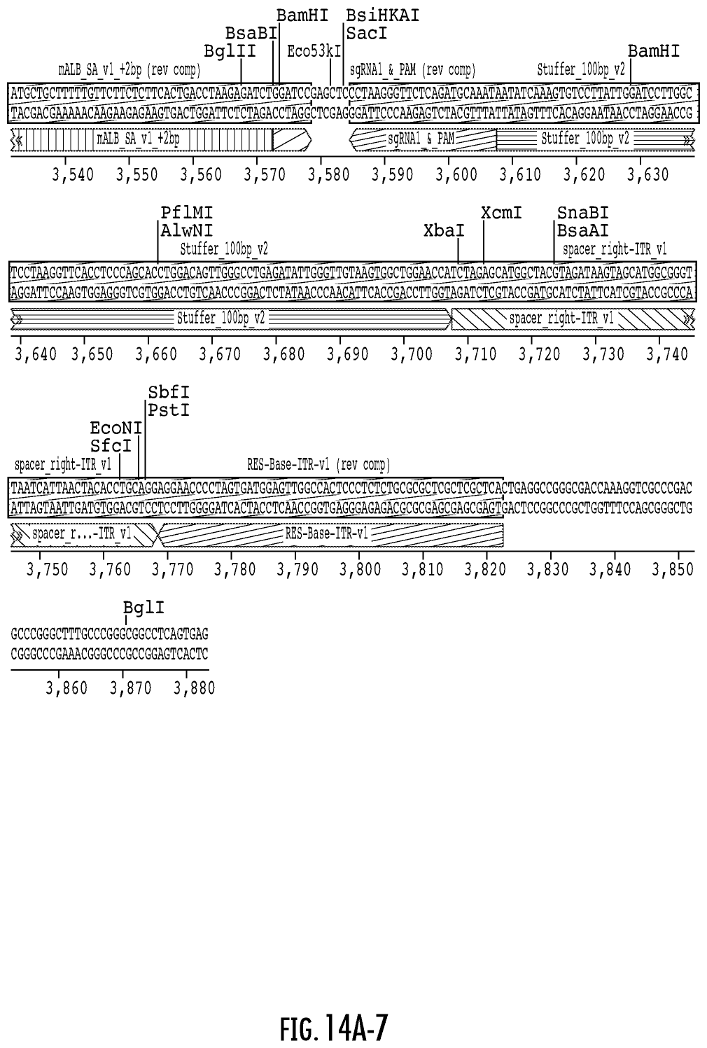

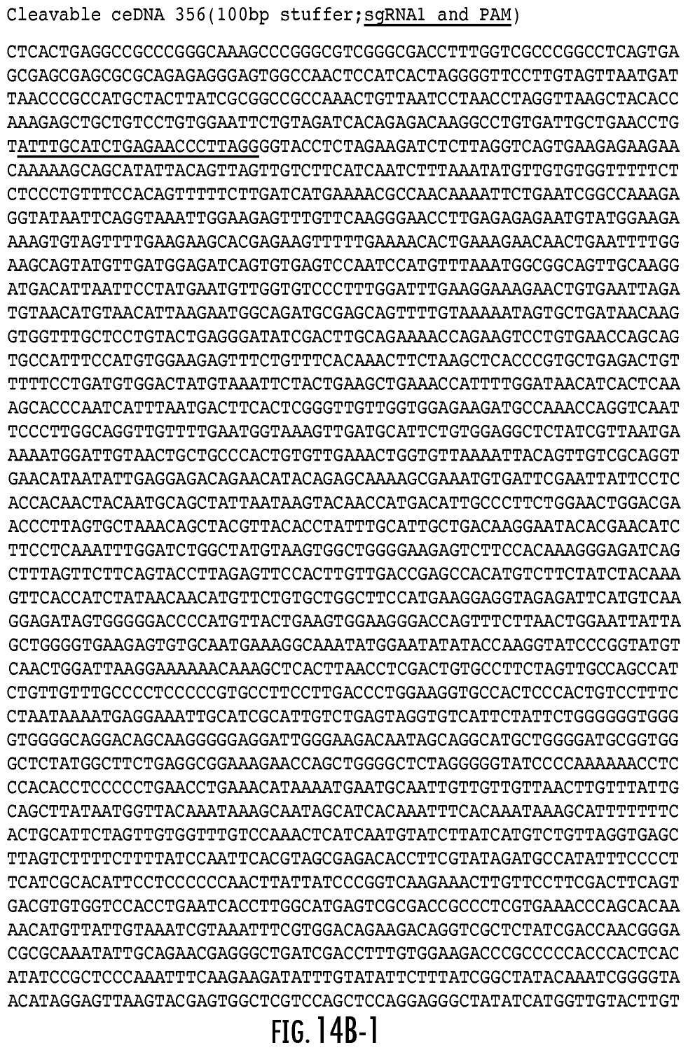

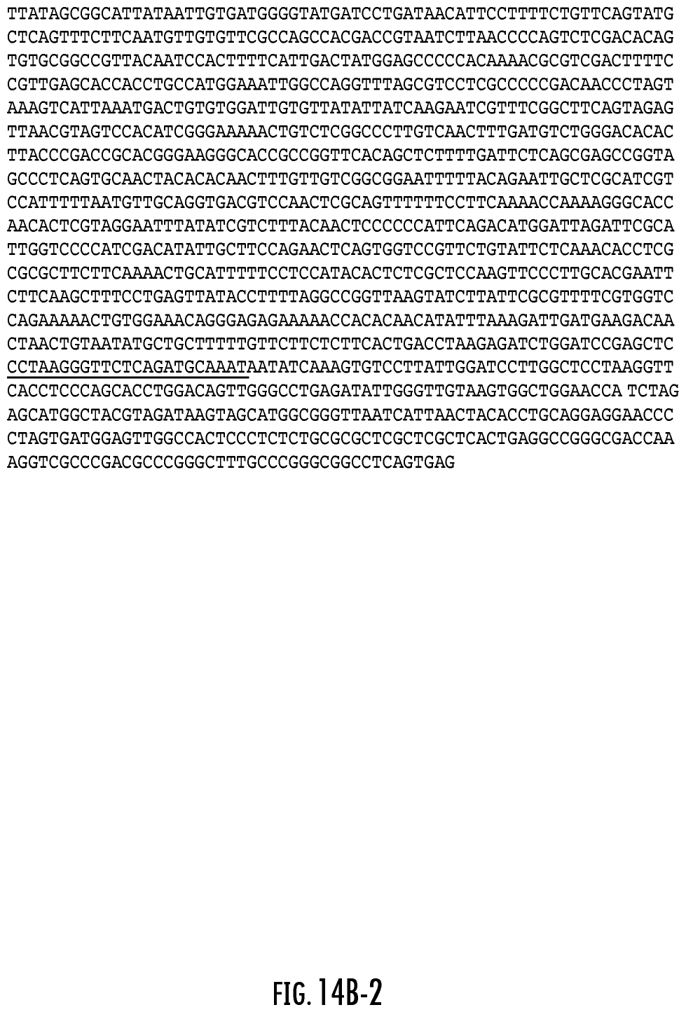

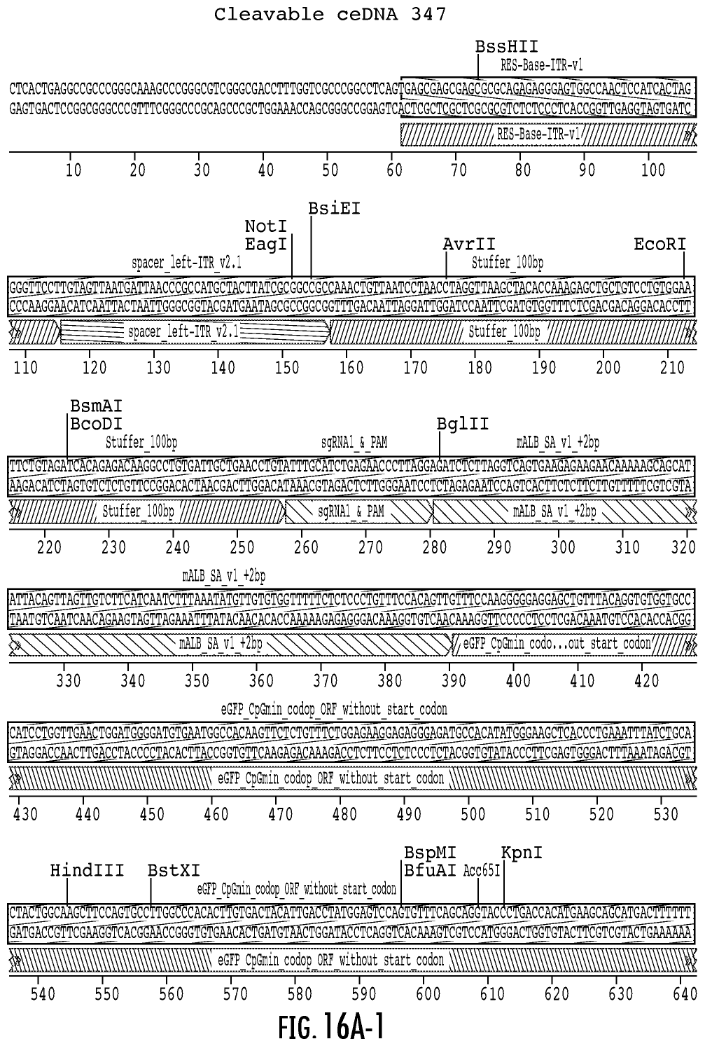

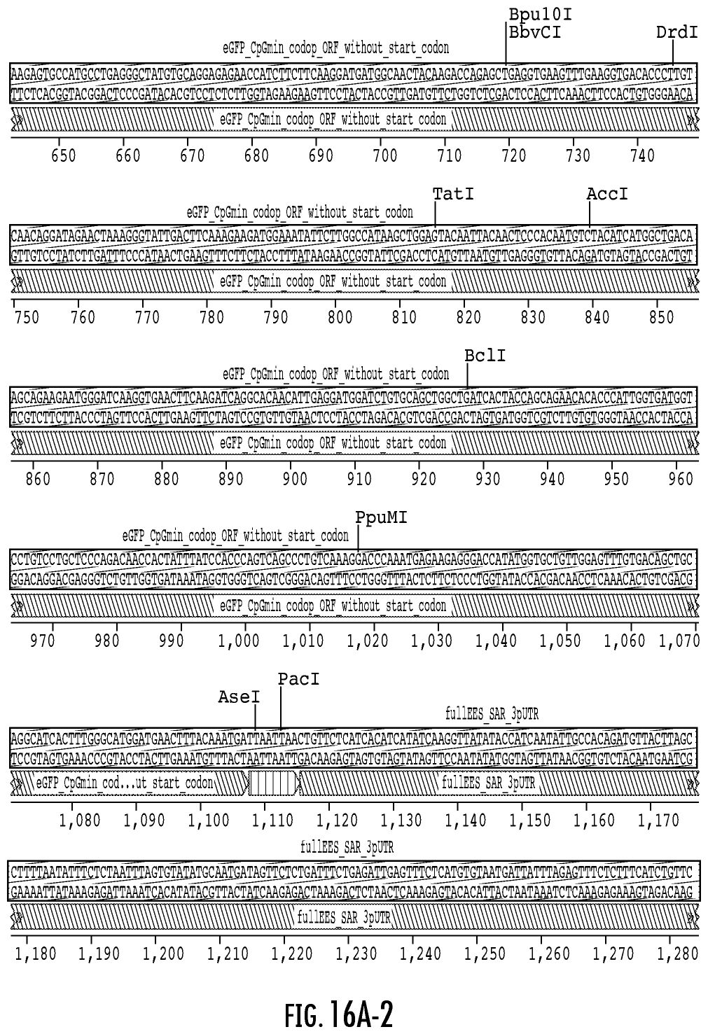

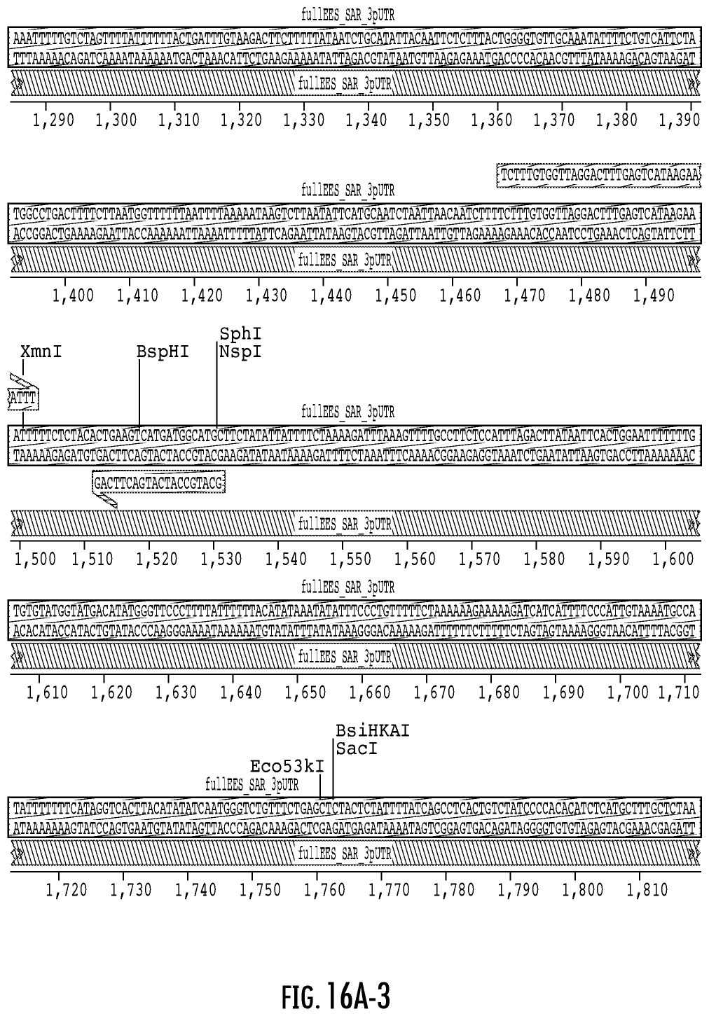

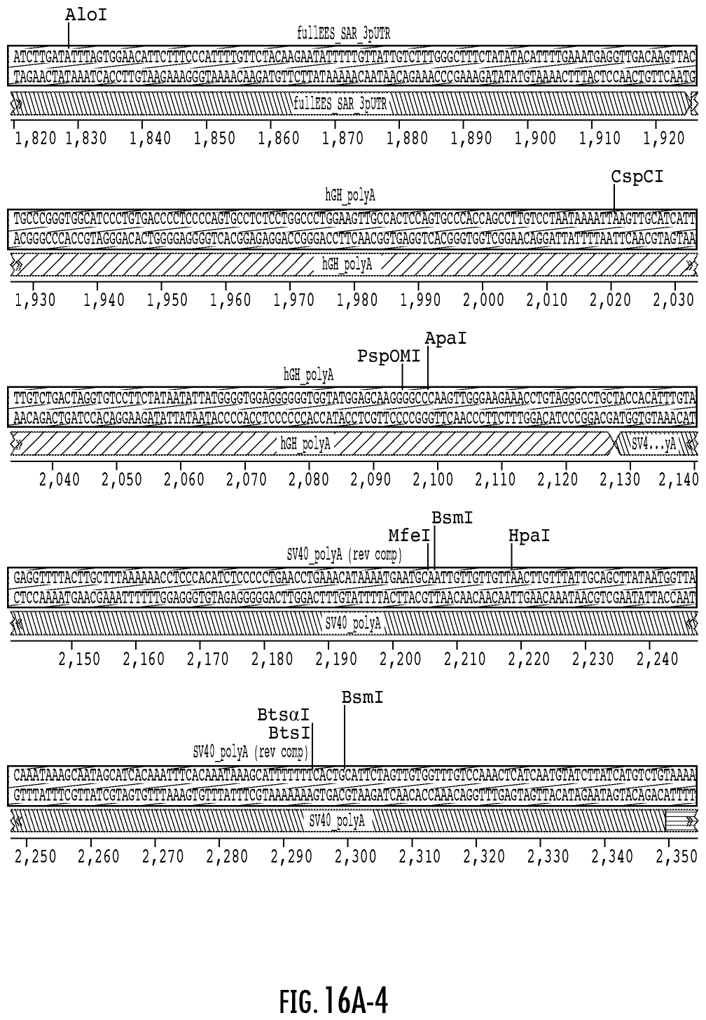

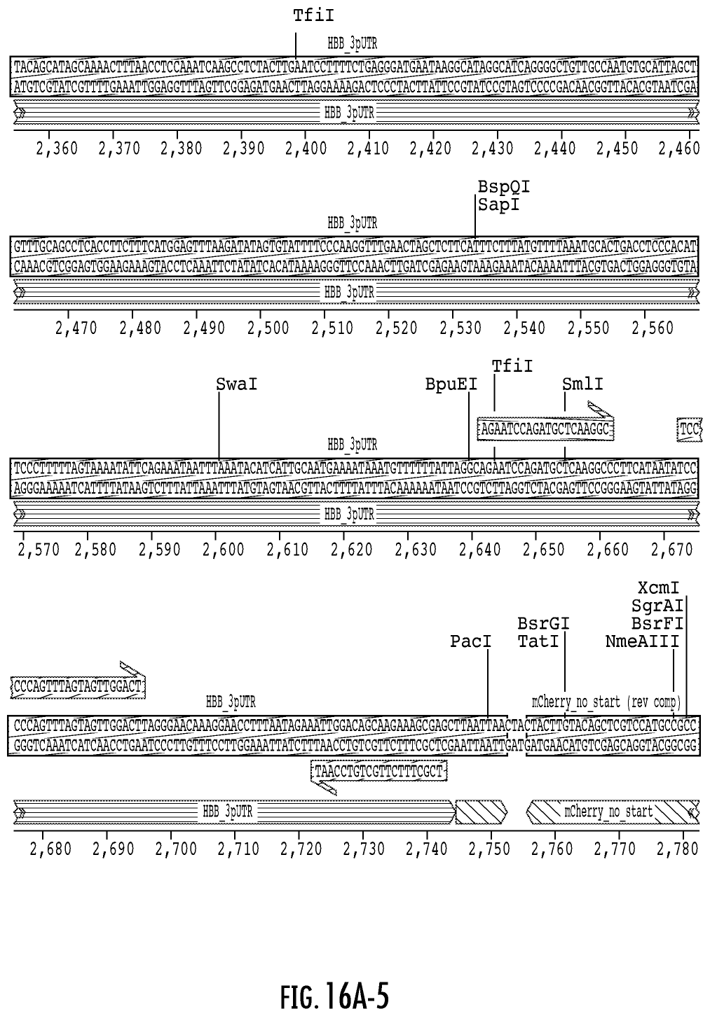

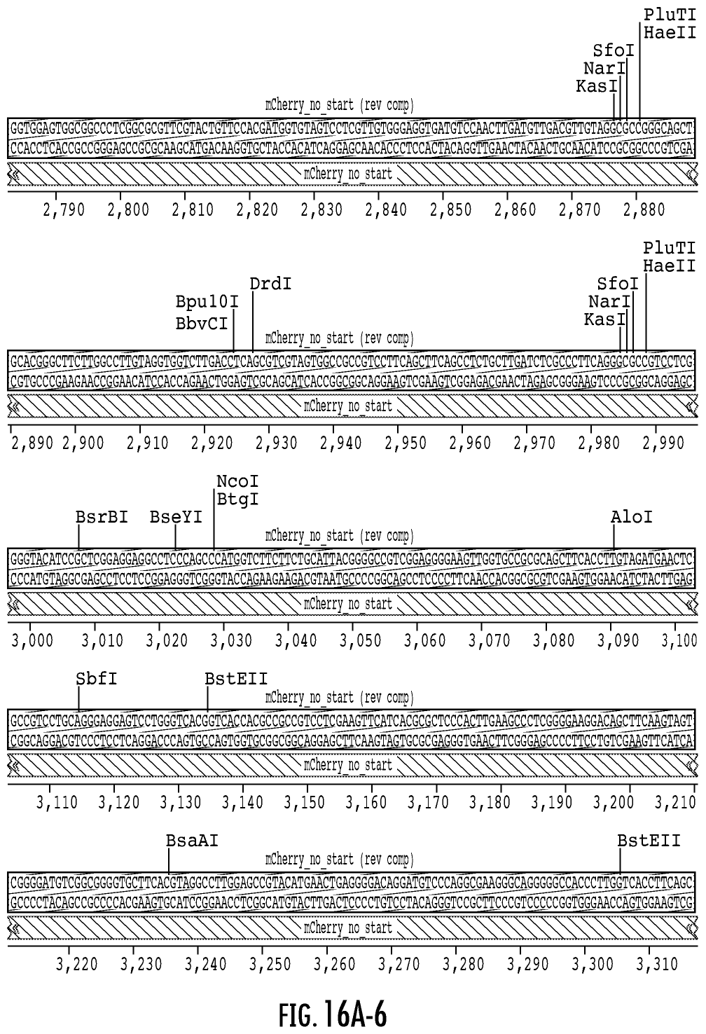

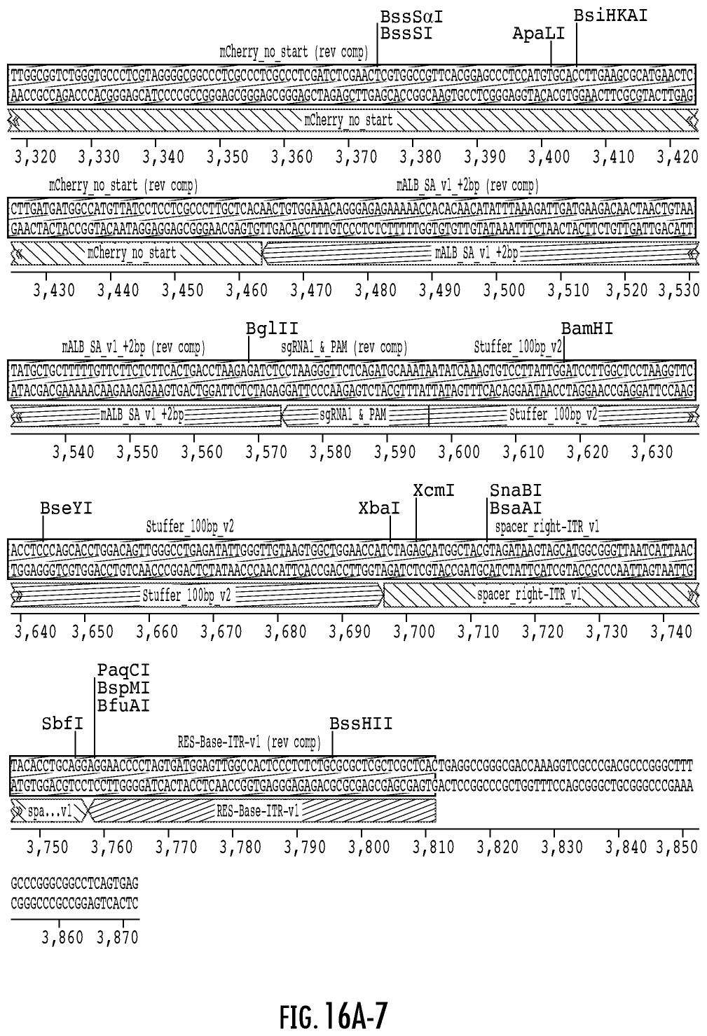

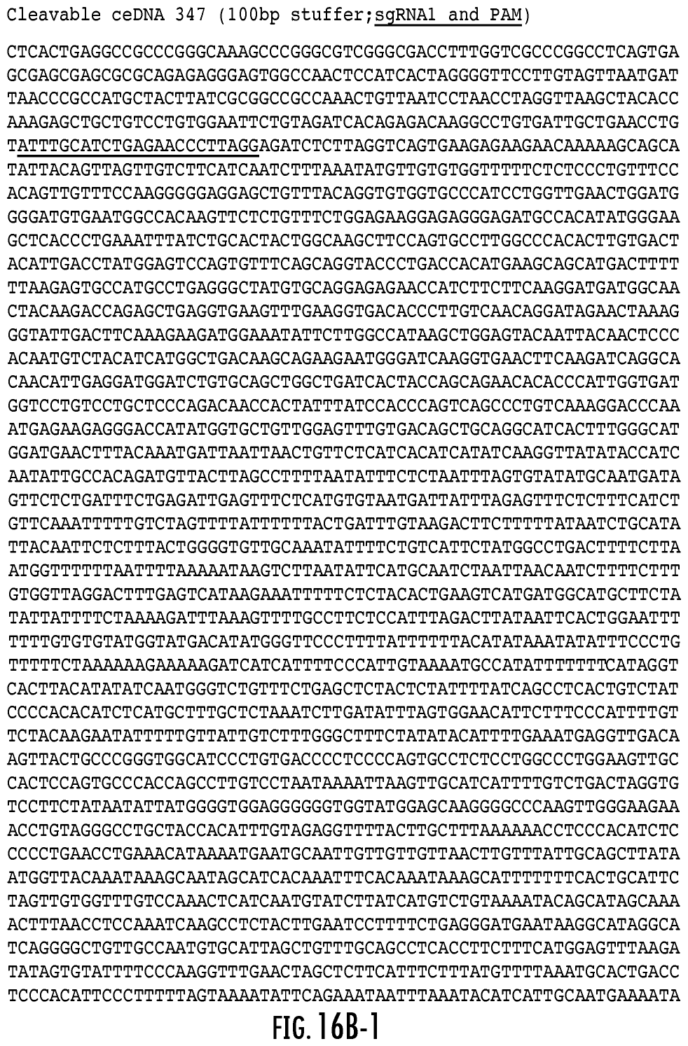

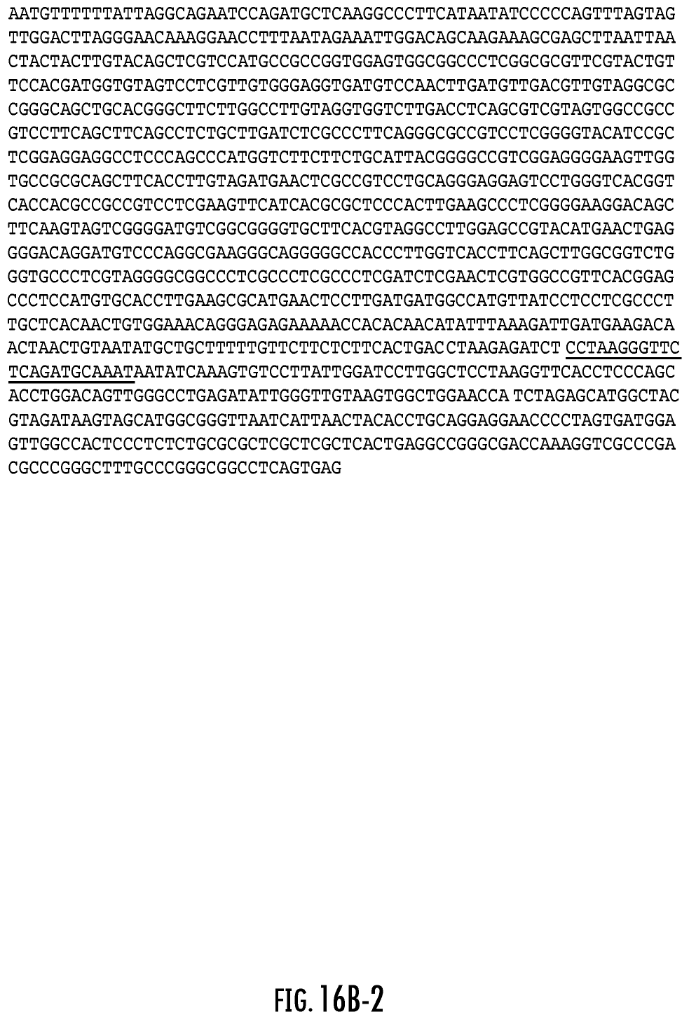

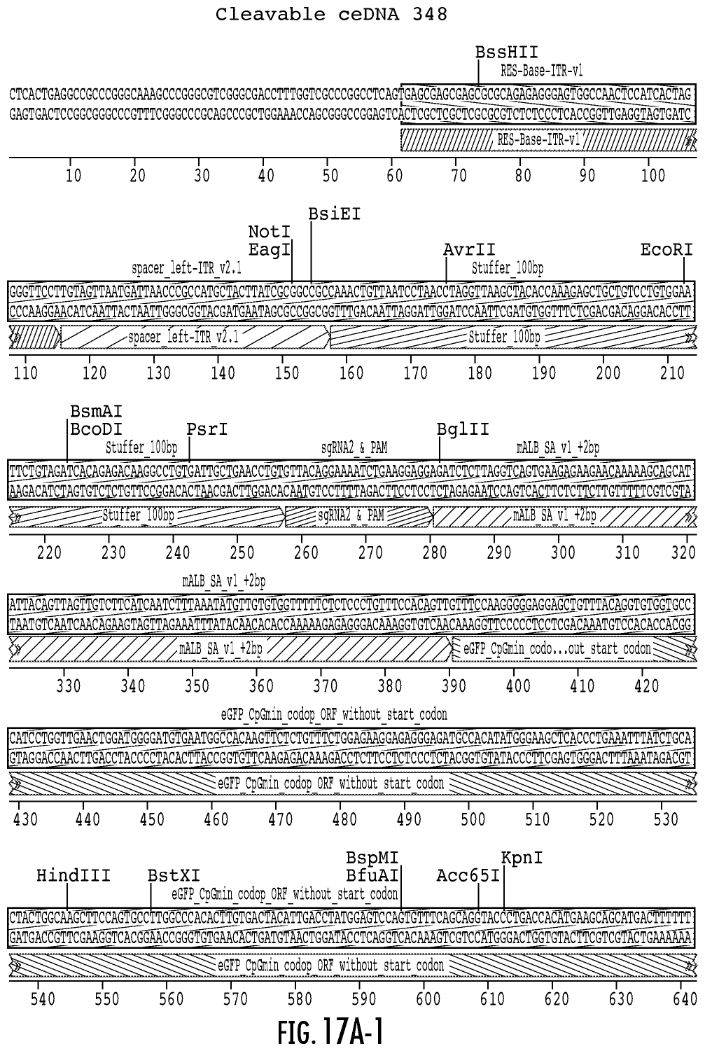

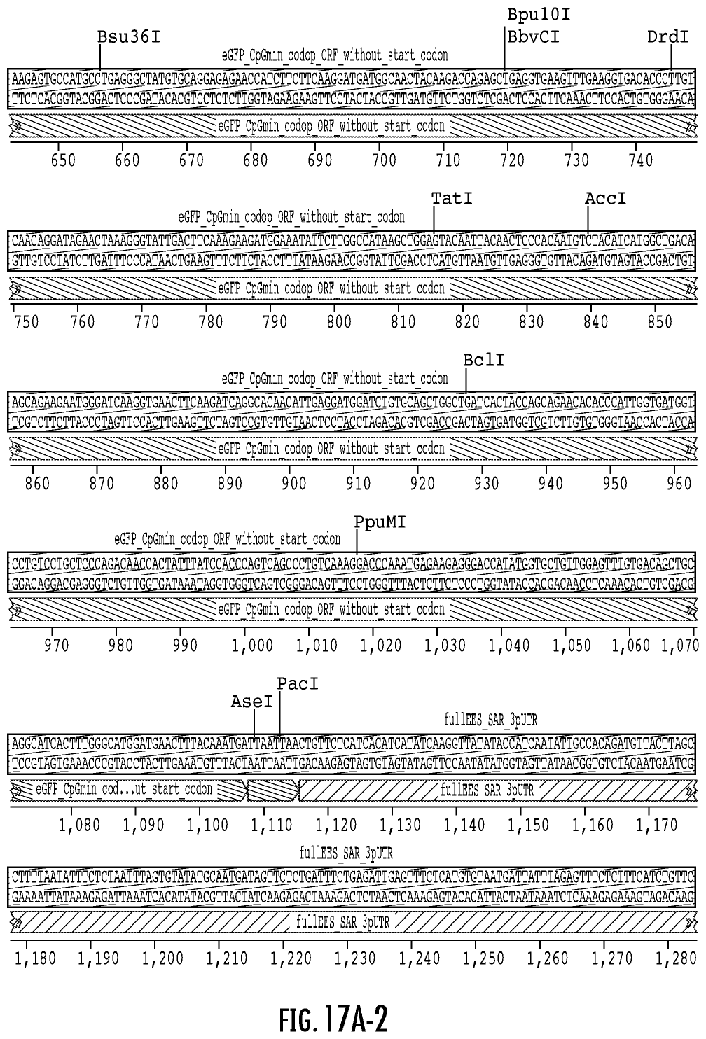

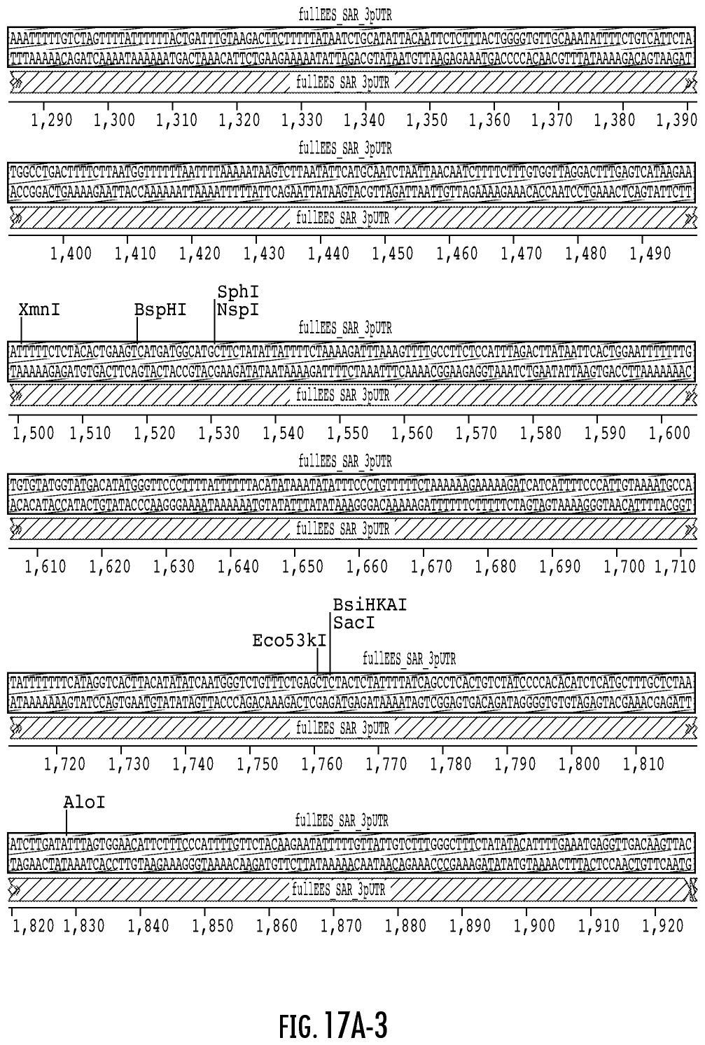

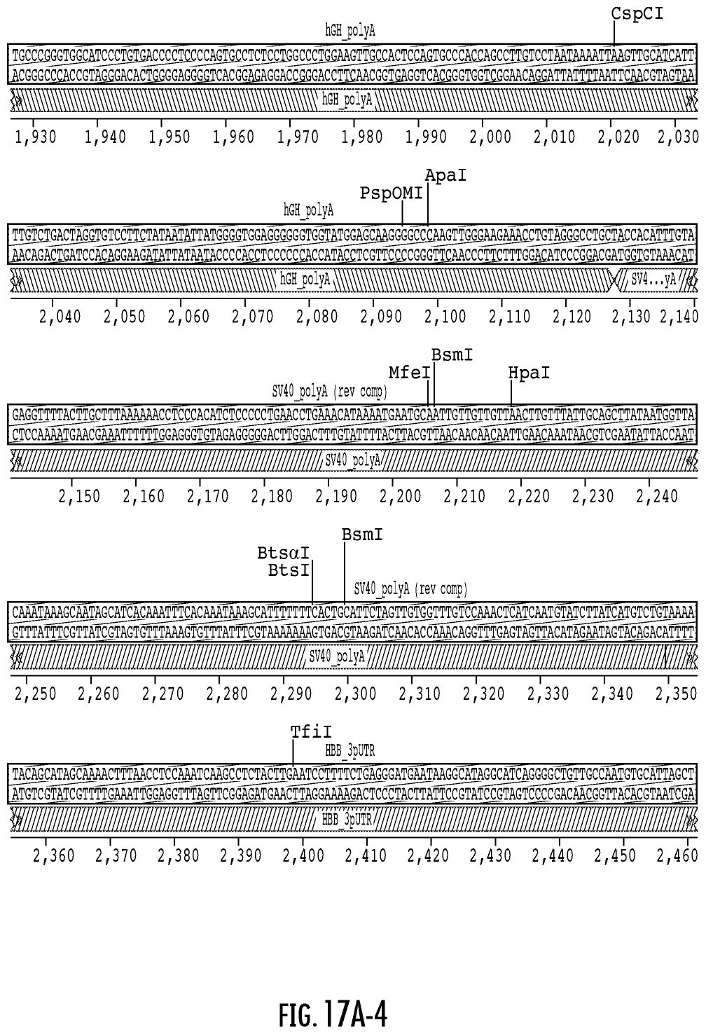

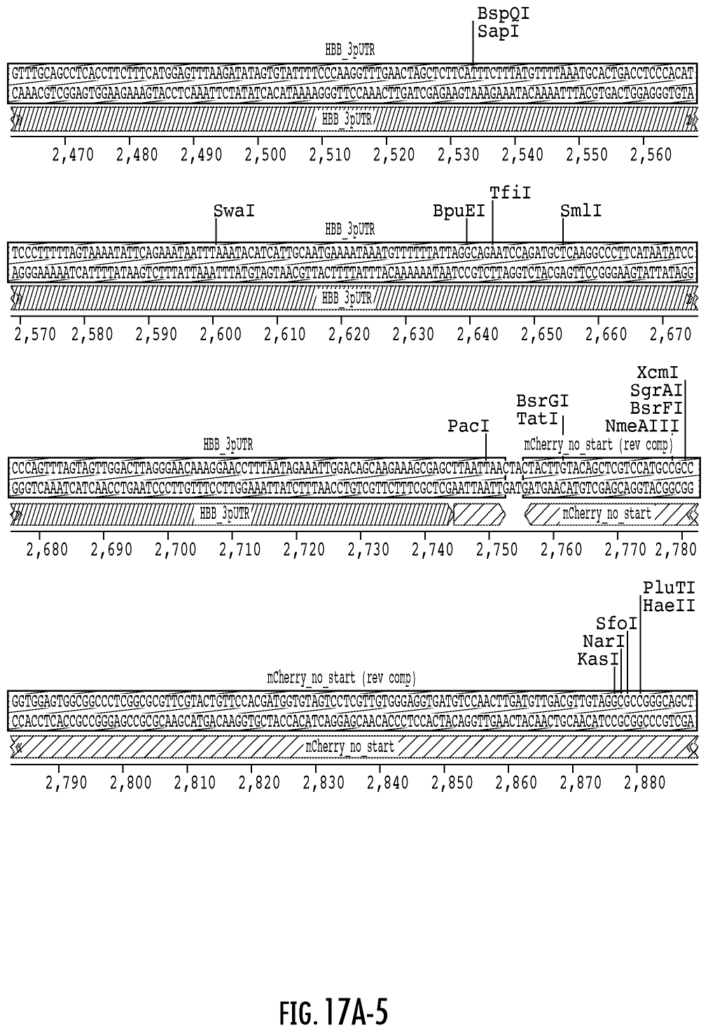

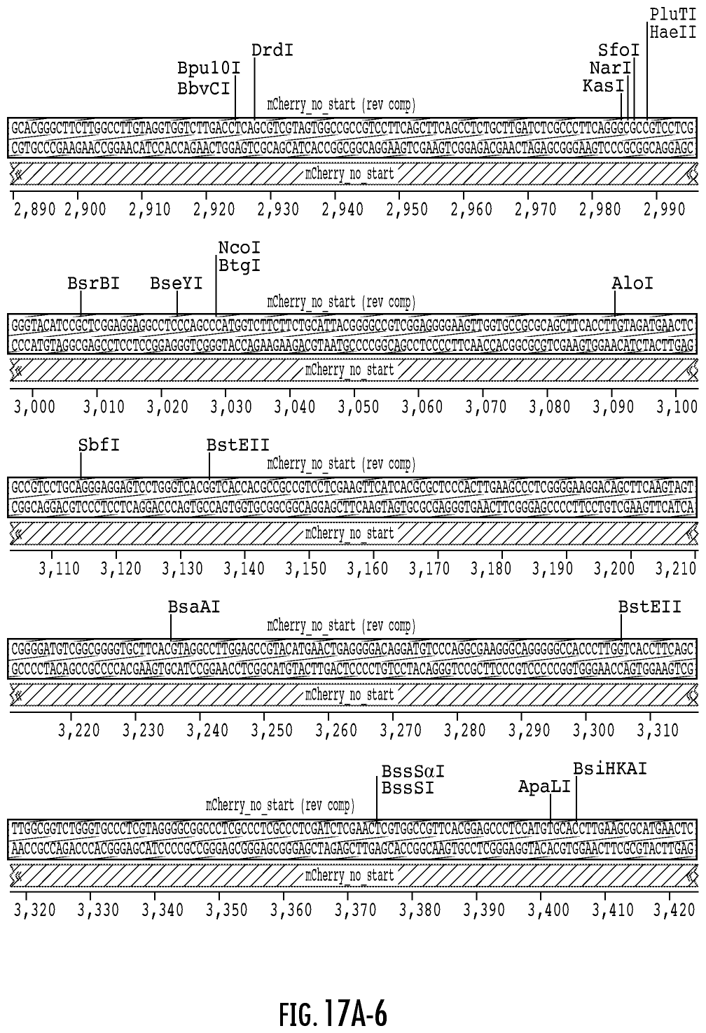

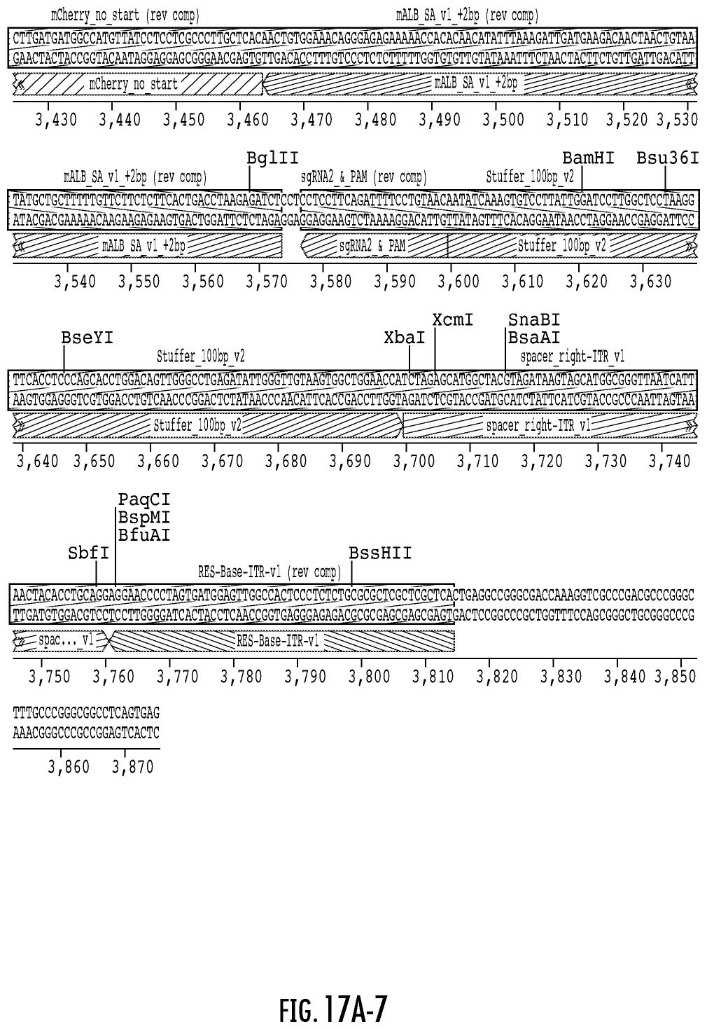

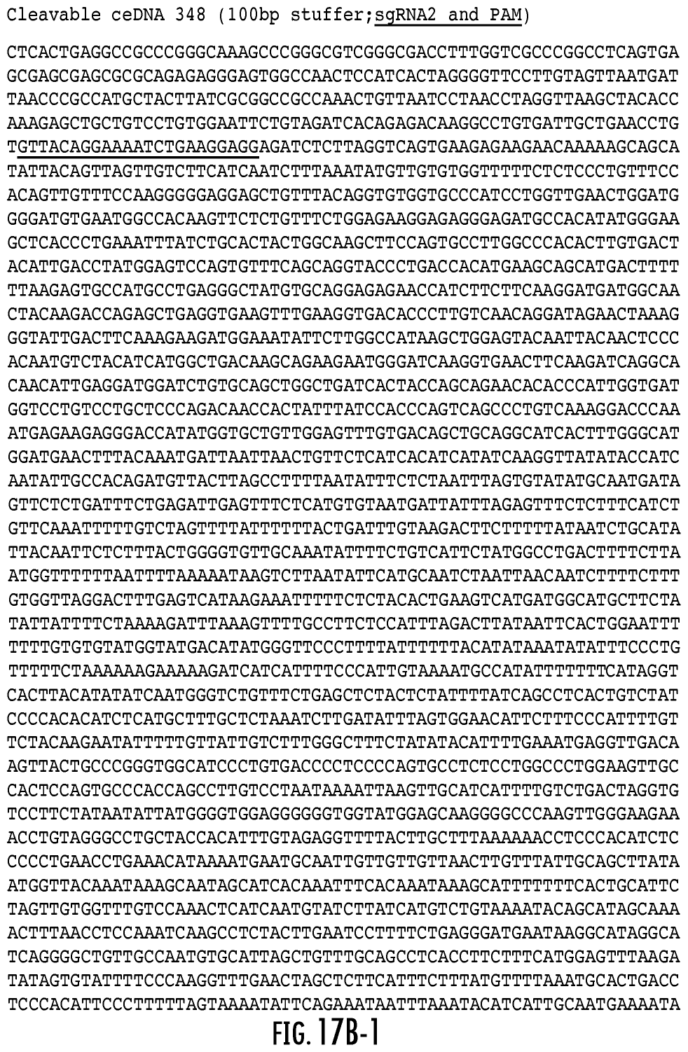

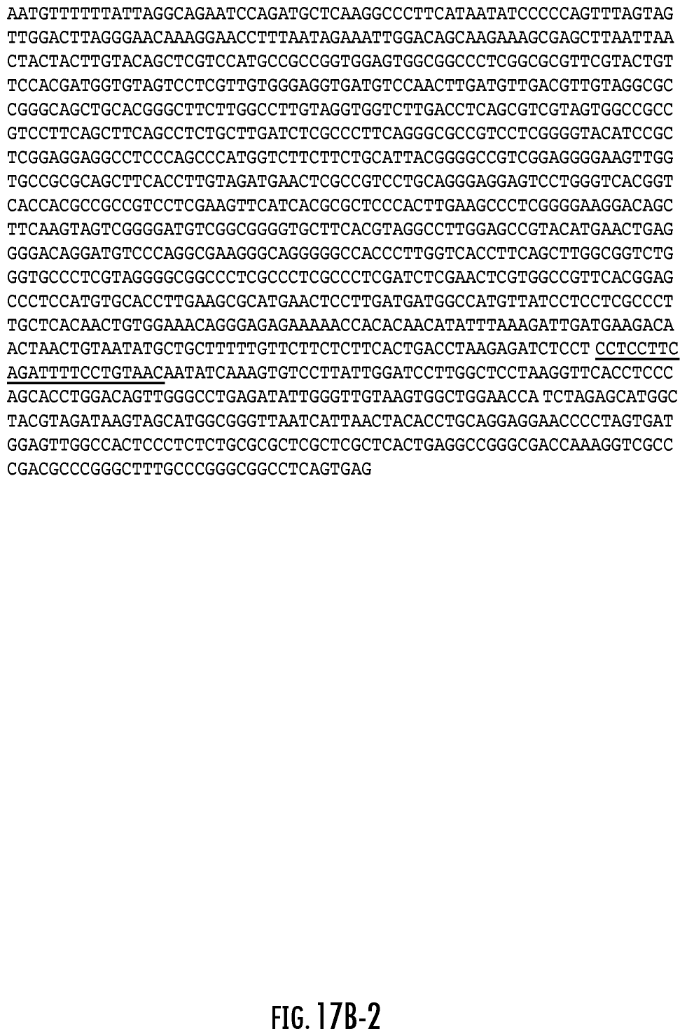

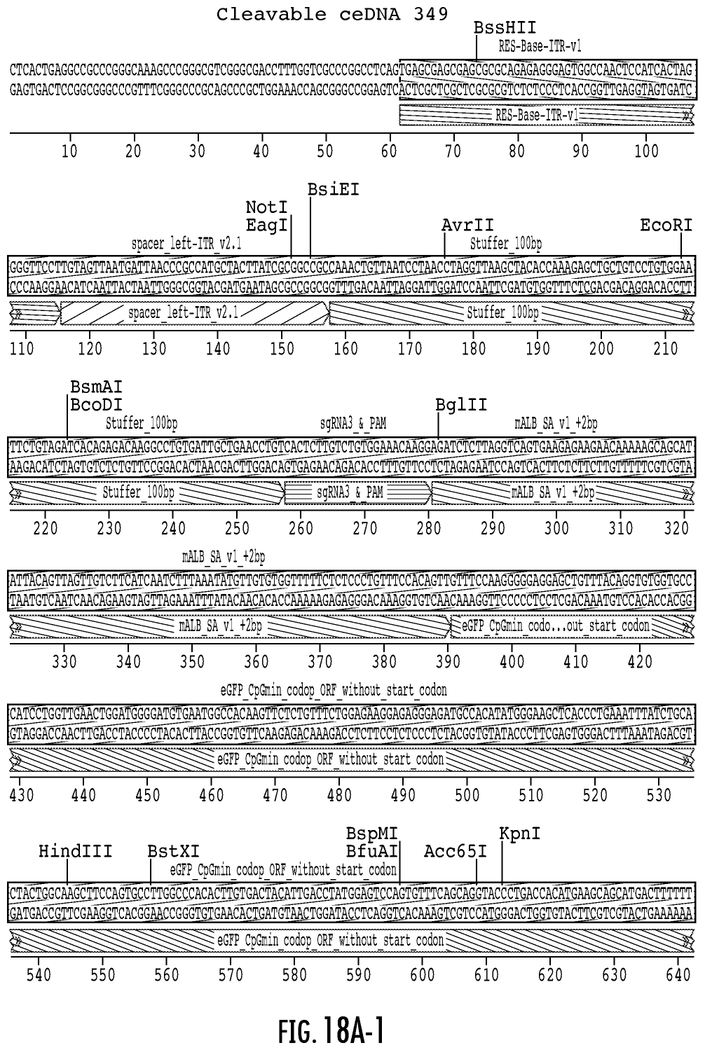

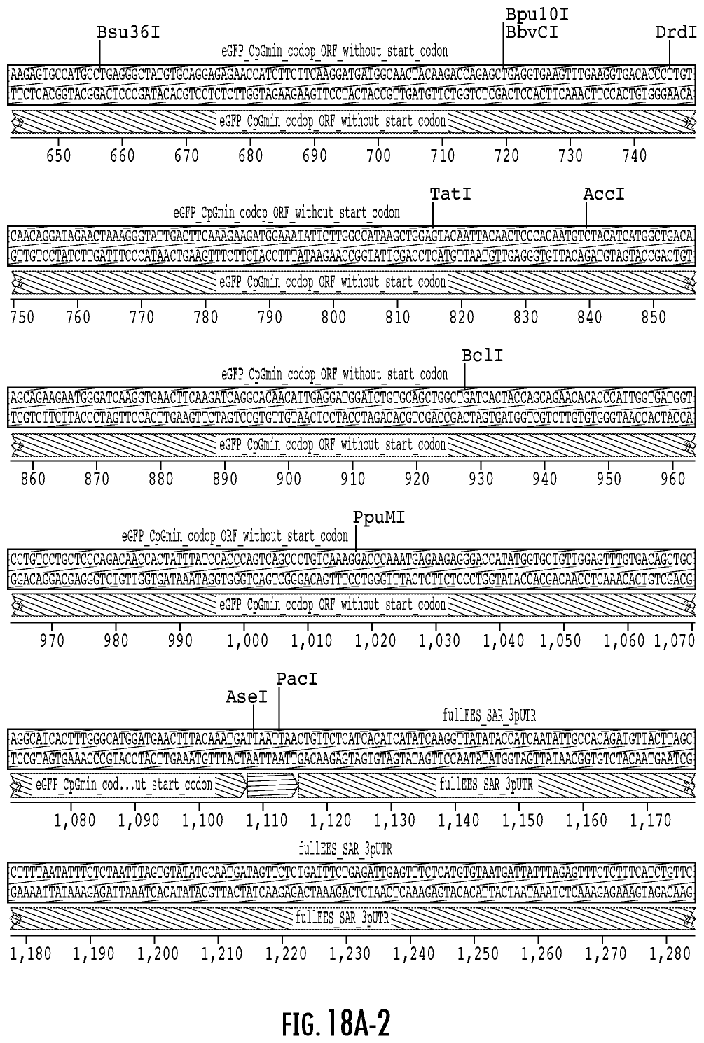

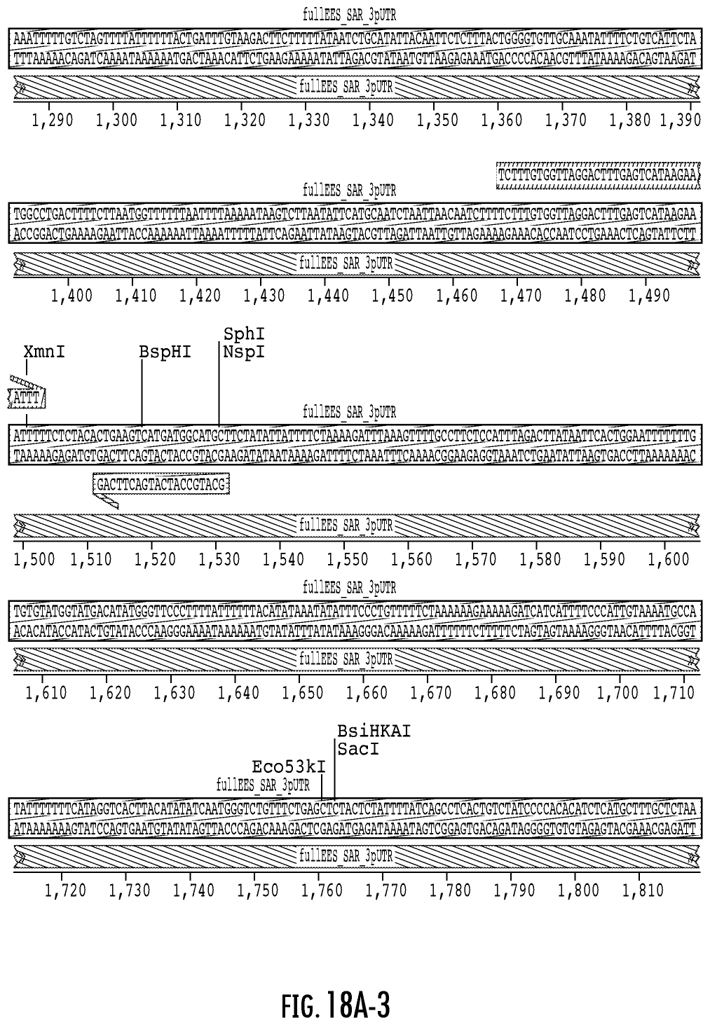

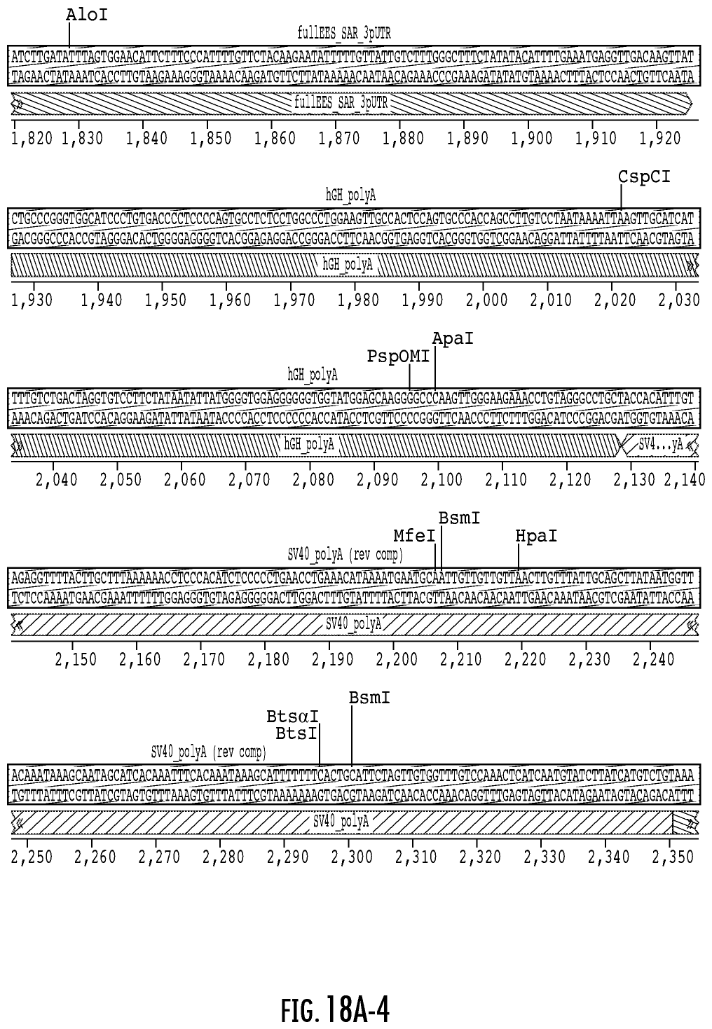

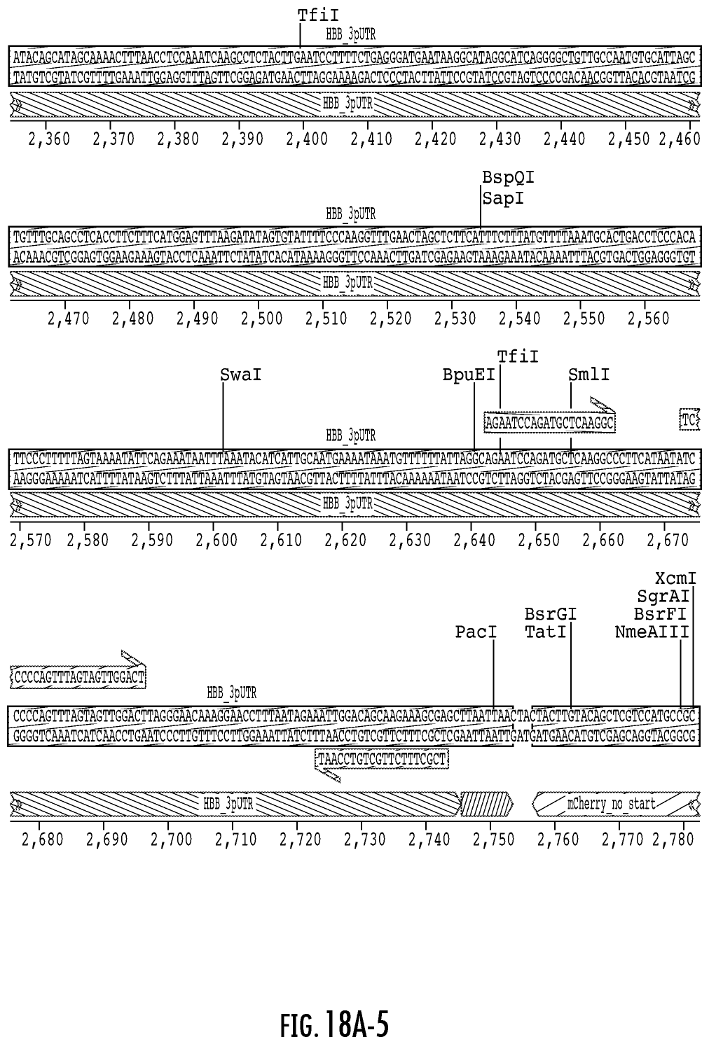

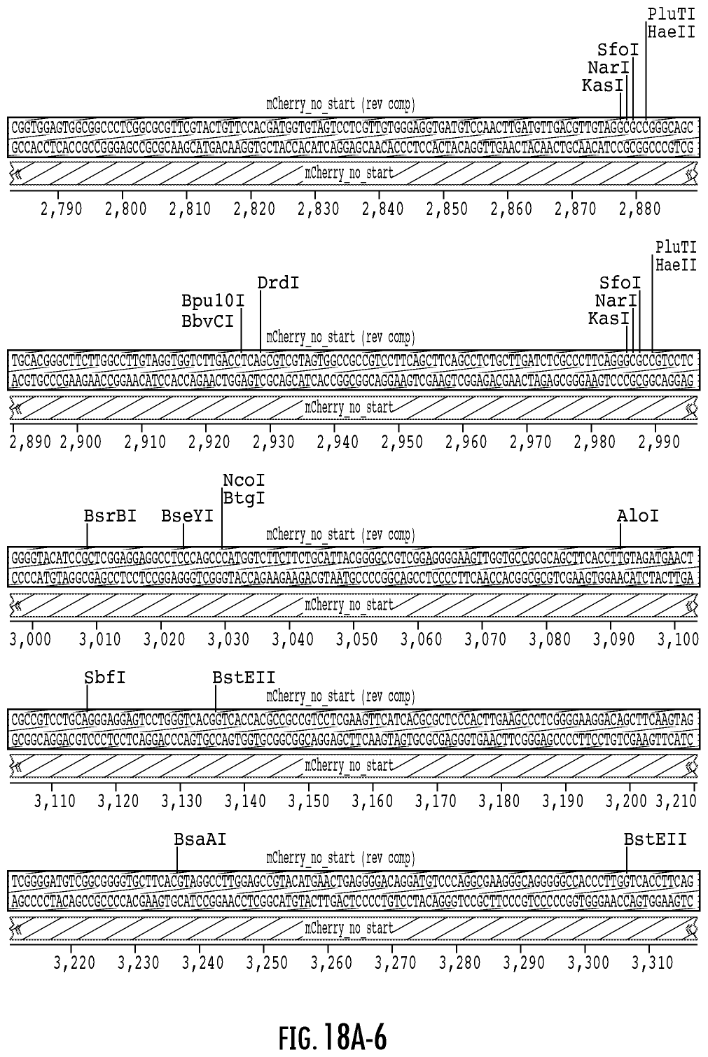

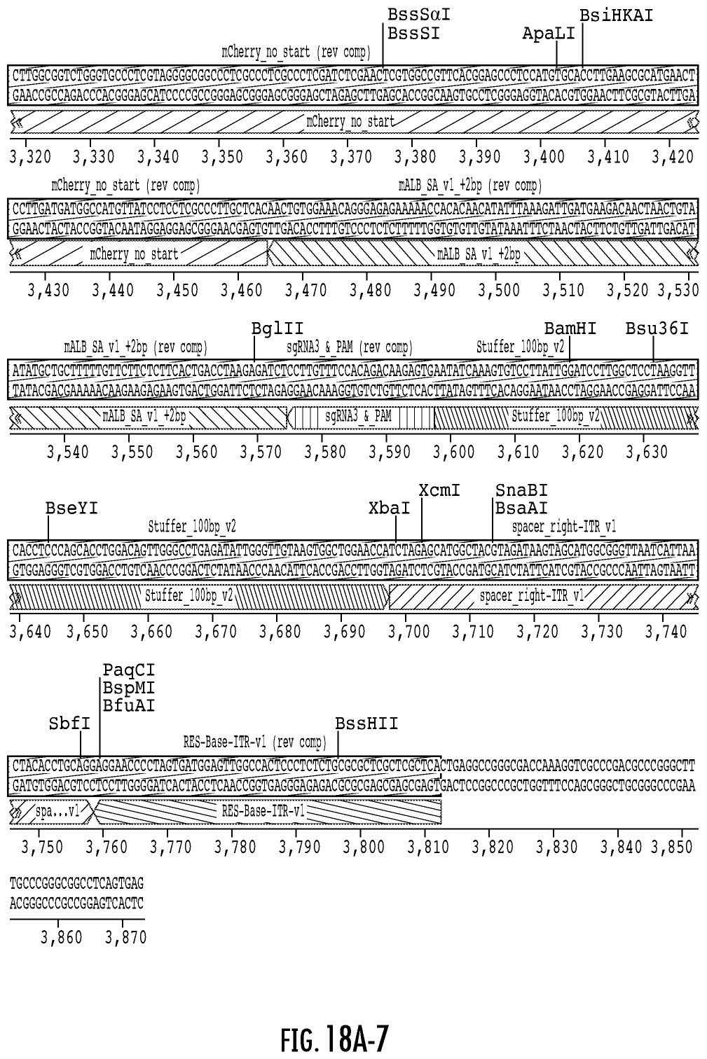

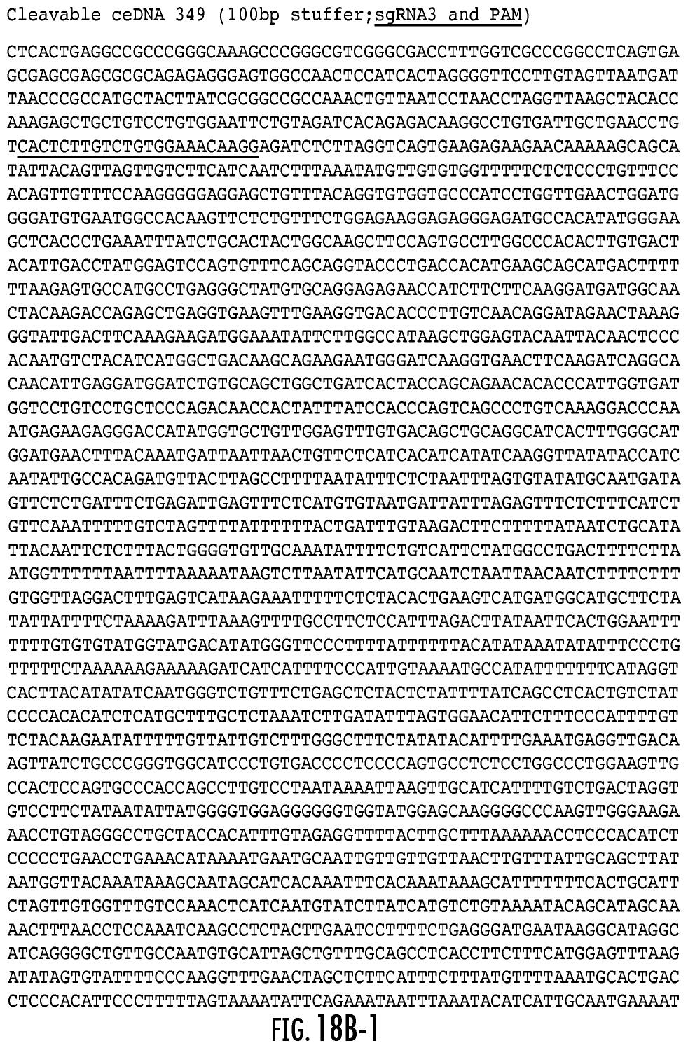

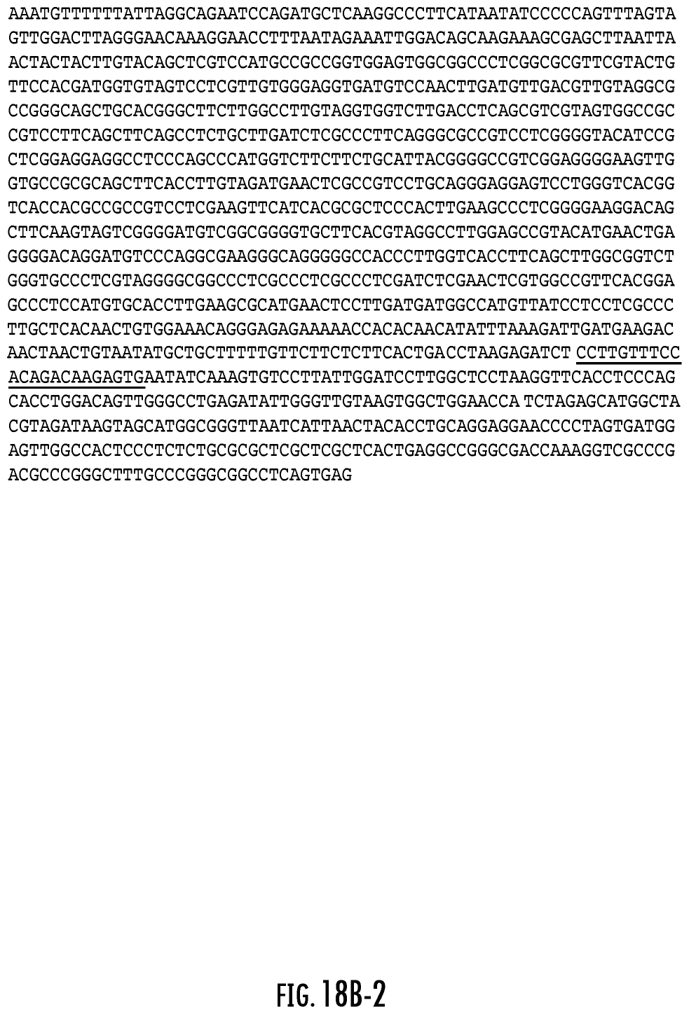

According to some embodiments, the cleavable ceDNA further comprises a second spacer (stuffer) sequence between the second gRNA TS and the second ITR. According to some embodiments, the cleavable ceDNA further comprises a first spacer sequence between the first ITR and the first gRNA TS. According to some embodiments, the second spacer sequence is between 1-200 nucleotides in length, for example between 1-175, between 1-150, between 1-100, between 50-150, between 150-200, between 100-200, between 50-100, between 25-50 nucleotides in length. According to some embodiments, the second spacer sequence is about 10, about 25, about 50, about 75, about 100, about 125, about 150, about 175, about 200 or about 300 nucleotides in length. According to some embodiments, the second spacer sequence is about 50 nucleotides in length. According to further embodiments, the second spacer sequence is about 100 nucleotides in length. According to further embodiments, the second spacer sequence is about 150 nucleotides in length. According to some embodiments, the first spacer (stuffer) sequence has at least 85%, 90%, 95%, 96%, 97%, 98% or 99% identity to a spacer sequence selected from SEQ ID NO: 45, as shown in FIG. 3B, SEQ ID NO: 49, as shown in FIG. 4B, SEQ ID NO: 53 as shown in FIG. 5B, SEQ ID NO: 57, as shown in FIG. 6B, SEQ ID NO: 61, as shown in FIG. 8B, SEQ ID NO: 65 as shown in FIG. 9B, SEQ ID NO: 69, as shown in FIG. 10B, SEQ ID NO: 73 as shown in FIG. 11B, SEQ ID NO: 77, as shown in FIG. 14B, SEQ ID NO: 81, as shown in FIG. 16B, SEQ ID NO: 85 as shown in FIG. 17B, and SEQ ID NO: 89, as shown in FIG. 18B. According to some embodiments, the second spacer (stuffer) sequence has at least 85%, 90%, 95%, 96%, 97%, 98% or 99% identity to a spacer sequence selected from SEQ ID NO: 46, as shown in FIG. 3B, SEQ ID NO: 50, as shown in FIG. 4B, SEQ ID NO: 54 as shown in FIG. 5B, SEQ ID NO: 58, as shown in FIG. 6B, SEQ ID NO: 62, as shown in FIG. 8B, SEQ ID NO: 66 as shown in FIG. 9B, SEQ ID NO: 70, as shown in FIG. 10B, SEQ ID NO: 74 as shown in FIG. 11B, SEQ ID NO: 78, as shown in FIG. 14B, SEQ ID NO: 82, as shown in FIG. 16B, SEQ ID NO: 86 as shown in FIG. 17B, and SEQ ID NO: 90, as shown in FIG. 18B.

The protospacer adjacent motif (PAM) is a short DNA sequence (usually 2-6 base pairs in length) that is about 3-4 nucleotides downstream from the DNA sequence targeted by Cas9. According to some embodiments, the PAM comprises a nucleic acid sequence selected from the group consisting of: 5′-NGG-3′, 5′-NGAAA-3′, 5′-NNG-3′, 5′-NGA-3′, 5′-NTAA-3′, 5′-NTG-3′, 5′-NNC-3′, 5′-NNAAC-3′, 5′-AGA-3′, 5′-NNNANNA-3′, 5′-NNANAA-3′, 5′-NNAAAA-3′, and 5′-AAAA-3′; and/or wherein the second PAM comprises a nucleic acid sequence selected from the group consisting of: 5′-NGG-3′, 5′-NGAAA-3′, 5′-NNG-3′, 5′-NGA-3′, 5′-NTAA-3′, 5′-NTG-3′, 5′-NNC-3′, 5′-NNAAC-3′, 5′-AGA-3′, 5′-NNNANNA-3′, 5′-NNANAA-3′, 5′-NNAAAA-3′, and 5′-AAAA-3′.

The cleavable ceDNA described herein may be used in a gene editing system.

According to some aspects, the disclosure provides a gene editing system comprising the cleavable ceDNA as described herein, at least one guide RNA (gRNA), and at least one Cas9 enzyme.

According to some embodiments, the at least one gRNA is capable of binding to both the first gRNA TS and the second gRNA TS. According to some embodiments, the gene editing system further comprises a second gRNA, wherein the second gRNA is capable of binding to the second gRNA TS, and wherein the at least one gRNA is capable of binding to the first gRNA TS.

These cleaved ceDNA may be used as donor sequences in combination with a nuclease enzyme and a guide RNA for insertion of transgenes into the genome, either through homology directed recombination (HDR), microhomology mediated end joining (MMEJ) or non-homology end joining (NHEJ) (also referred to as homology-independent targeted integration (HITI)).

The cleavable ceDNA vectors of the present disclosure are compatible with the host cell into which the cleavable ceDNA vector is to be introduced. In certain embodiments, the cleavable ceDNA vectors may be linear. In certain embodiments, the cleavable ceDNA vectors may exist as an extrachromosomal entity. In certain embodiments, the cleavable ceDNA vectors of the present disclosure may contain an element(s) that permits integration of a donor sequence into the host cell's genome. As used herein “donor sequence” and “transgene” and “heterologous nucleotide sequence” are synonymous.

### A. DNA Endonucleases

The gene editing systems of the present disclosure may contain a nucleotide sequence that encodes a nuclease, such as a sequence-specific nuclease. Sequence-specific or site-specific nucleases can be used to introduce site-specific double strand breaks or nicks at targeted genomic loci. This nucleotide cleavage, e.g., DNA or RNA cleavage, stimulates the natural repair machinery, e.g., DNA repair machinery, leading to one of two possible repair pathways. In some aspects, the break will be repaired by non-homologous end joining (NHEJ), an error-prone repair pathway that leads to small insertions or deletions of DNA (see e.g., Suzuki et al. Nature 540:144-149 (2016), the contents of which are incorporated by reference in its entirety). This method can be used to intentionally disrupt, delete, or alter the reading frame of targeted gene sequences. In other aspects, if a repair template is provided in addition to the nuclease, then the cellular machinery will repair the break by homologous recombination (HDR), which is enhanced several orders of magnitude in the presence of DNA cleavage, or by insertion of the donor sequence via NHEJ.

The methods can be used to introduce specific changes in the DNA sequence at target sites. The term “site-specific nuclease” as used herein refers to an enzyme capable of specifically recognizing and cleaving a particular DNA sequence. The site-specific nuclease may be engineered. Examples of engineered site-specific nucleases include zinc finger nucleases (ZFNs), TAL effector nucleases (TALENs), meganucleases, and CRISPR/Cas9-enzymes and engineered derivatives. As will be appreciated by those of skill in the art, the endonucleases necessary for gene editing can be expressed transiently, as there is generally no further need for the endonuclease once gene editing is complete. Such transient expression can reduce the potential for off-target effects and immunogenicity. Transient expression can be accomplished by any known means in the art, and may be conveniently effected using a regulatory switch as described herein.

In some embodiments, the nucleotide sequence encoding the nuclease is cDNA. Non-limiting examples of sequence-specific nucleases include RNA-guided nuclease, zinc finger nuclease (ZFN), a transcription activator-like effector nuclease (TALEN) or a meganuclease. Non-limiting examples of suitable RNA-guided nucleases include CRISPR enzymes as described herein.

The nucleases described herein can be altered, e.g., engineered to design sequence specific nuclease (see e.g., U.S. Pat. No. 8,021,867). Nucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, nuclease with site specific cutting characteristics can be obtained using commercially available technologies e.g., Precision BioSciences' Directed Nuclease Editor™ genome editing technology.

In certain embodiments, for example when using a promoterless ceDNA construct comprising a homology directed recombination (HDR) repair template, the guide RNA and/or Cas enzyme, or any other nuclease, are delivered in trans, e.g., by administering i) a nucleic acid encoding a guide RNA, ii) or an mRNA encoding a the desired nuclease, e.g., Cas enzyme, or other nuclease iii) or by administering a ribonucleotide protein (RNP) complex comprising a Cas enzyme and a guide RNA, or iv) e.g., delivery of recombinant nuclease proteins by vector, e.g., viral, plasmid, or another ceDNA vector. In certain aspects, the molecules delivered in trans are delivered by means of one or more additional ceDNA vectors which can be co-administered or administered sequentially to the first ceDNA vector.

Accordingly, in one embodiment, a gene editing system can comprise an endonuclease (e.g., Cas9) that is transcriptionally regulated by an inducible promoter. In some embodiments, the endonuclease is on a separate ceDNA vector, which can be administered to a subject with a cleavable ceDNA or a cleaved ceDNA comprising homology arms and a donor sequence, which can optionally also comprise guide RNA (sgRNAs). In alternative embodiments, the endonuclease can be on an all-in-one ceDNA vector as described herein.

In some embodiments, the gene editing systems comprise an endonuclease as described herein under control of a promoter. Non-limiting examples of inducible promoters include chemically-regulated promoters, which regulate transcriptional activity by the presence or absence of, for example, alcohols, tetracycline, steroids, metal, and pathogenesis-related proteins (e.g., salicylic acid, ethylene, and benzothiadiazole), and physically-regulated promoters, which regulate transcriptional activity by, for example, the presence or absence of light and low or high temperatures. Modulation of the inducible promoter allows for the turning off or on of gene-editing activity of a cleavable ceDNA vector. Inducible Cas9 promoters are further reviewed, for example in Cao J., et al. Nucleic Acids Research. 44(19)2016, and Liu K I, et al. Nature Chemical Biol. 12: 90-987 (2016), which are incorporated herein in their entireties.

In one embodiment, the gene editing systems described herein further comprise a second endonuclease that temporally targets and inhibits the activity of the first endonuclease (e.g., Cas9). Endonucleases that target and inhibit the activity of other endonucleases can be determined by those skilled in the art. In another embodiment, the cleavable ceDNA vector described herein further comprises temporal expression of an “anti-CRISPR gene” (e.g., L. monocytogenes ArcIIa). As used herein, “anti-CRISPR gene” refers to a gene shown to inhibit the commonly used S. pyogenes Cas9. In another embodiment, the second endonuclease that targets and inhibits the activity of the first endonuclease activity, or the anti-CRISPR gene, is comprised in a second ceDNA vector that is administered after the desired gene-editing is complete. Alternatively, the second endonuclease targets and inhibits a gene of interest, for example, a gene that has been transcriptionally enhanced as described herein.

A cleavable ceDNA vector or composition thereof, as described herein, can include a nucleotide sequence encoding a transcriptional activator that activates a target gene. For example, the transcriptional activator may be engineered. For example, an engineered transcriptional activator may be a CRISPR/Cas9-based system, a zinc finger fusion protein, or a TALE fusion protein. The CRISPR/Cas9-based system, as described above, may be used to activate transcription of a target gene with RNA. The CRISPR/Cas9-based system may include a fusion protein, as described above, wherein the second polypeptide domain has transcription activation activity or histone modification activity. For example, the second polypeptide domain may include VP64 or p300. Alternatively, the transcriptional activator may be a zinc finger fusion protein. The zinc finger targeted DNA-binding domains, as described above, can be combined with a domain that has transcription activation activity or histone modification activity. For example, the domain may include VP64 or p300. TALE fusion proteins may be used to activate transcription of a target gene. The TALE fusion protein may include a TALE DNA-binding domain and a domain that has transcription activation activity or histone modification activity. For example, the domain may include VP64 or p300.

Another method for modulating gene expression at the transcription level is by targeting epigenetic modifications using modified DNA endonucleases as described herein. Modulation of gene expression at the epigenetic level has the advantage of being inherited by daughter cells at a higher rate than the activation/inhibition achieved using CRISPRa or CRISPRi. In one embodiment, dCas9 fused to a catalytic domain of p300 acetyltransferase can be used with the methods and compositions described herein to make epigenetic modifications (e.g., increase histone modification) to a desired region of the genome. Epigenetic modifications can also be achieved using modified TALEN constructs, such as a fusion of a TALEN to the Tet I demethylase catalytic domain (see e.g., Maeder et al. Nature Biotechnology 31(12):1137-42 (2013)) or a TAL effector fused to LSD1 histone demethylase (Mendenhall et al. Nature Biotechnology 31(12):1133-6 (2013)).

### (i) Modified DNA Endonucleases, Nuclease-Dead Cas9 and Uses Thereof

Unlike viral vectors, the ceDNA vectors as described herein do not have a capsid that limits the size or number of nucleic acid sequences, effector sequences, regulatory sequences etc. that can be delivered to a cell. Accordingly, ceDNA vectors as described herein can comprise nucleic acids encoding nuclease-dead DNA endonucleases, nickases, or other DNA endonucleases with modified function (e.g., unique PAM binding sequence) for enhanced production of a desired vector and/or delivery of the vector to a cell. Such ceDNA vectors can also include promoter sequences and other regulatory or effector sequences as desired. Given the lack of size constraint, one of skill in the art will readily understand that, for example, that expression of a desired nuclease with modified function, and optionally, at least one guide RNA can be from nucleic acid sequences on the same vector and can be under the control of the same or different promoters. It is also contemplated herein that at least two different modified endonucleases can be encoded in the same vector, for example, for multiplexed gene expression modulation (see “Multiplexed gene expression modulation” section herein) and under the control of the same or different promoters. Thus, one of skill in the art could combine the desired functionality of at least two different Cas9 endonucleases (e.g., at least 3, at least 4, at least 5, at least 6, at least 7, at least 8, at least 9, at least 10, or more) as desired including, for example, temporally regulated expression of at least two different modified endonucleases by one or more inducible promoters. Alternatively, the gene editing systems described herein can comprise either a RNP comprising a nuclease and at least one guide RNA, in addition to the cleavable or cleaved ceDNA; or a nucleic acid encoding a nuclease and at least one guide RNA, in addition to the cleavable or cleaved ceDNA.

In some embodiments, a DNA endonuclease for use with the methods and compositions described herein, can be modified such that the DNA endonuclease retains DNA binding activity e.g., at a target site of the genome determined by a guide RNA sequence but does not retain cleavage activity (e.g., nuclease dead Cas9 (dCas9)) or has reduced cleavage activity (e.g., by at least 10%, at least 20%, at least 30%, at least 40%, at least 50%, at least 60%, at least 70%, at least 80%, at least 90%, at least 95%, at least 99%) as compared to the unmodified DNA endonuclease (e.g., Cas9 nickase). In some embodiments, a modified DNA endonuclease is used herein to inhibit expression of a target gene. For example, since a modified DNA endonuclease retains DNA binding activity, it can prevent the binding of RNA polymerase and/or displace RNA polymerase, which in turn prevents transcription of the target gene. Thus, expression of a gene product (e.g., mRNA, protein) from the desired gene is prevented.

For example, a “deactivated Cas9 (dCas9),” “nuclease dead Cas9” or an otherwise inactivated form of Cas9 can be introduced with a guide RNA that directs binding to a specific gene. Such binding can reduce in inhibition of expression of the target gene, if desired. In some embodiments, one may want to have the ability to reverse such gene expression inhibition. This can be achieved, for example, by providing different guide RNAs to the dead Cas9 protein to weaken the binding of Cas9 to the genomic site. Such reversal can occur in an iterative fashion where at least two or a series of guide RNAs designed to decrease the stability of the dead Cas9 binding are administered in succession. For example, each successive guide RNA can increase the instability from the degree of instability/stability of dead Cas9 binding produced by the guide RNA in the previous iteration. Thus, in some embodiments, one can use a dCas9 directed to a target gene sequence with a guide RNA to “inactivate a desired gene,” without cleavage of the genomic sequence, such that the gene of interest is not expressed in a functional protein form. In alternative embodiments, a guide RNA can be designed such that the stability of the dCas9 binding is reduced, but not eliminated. That is, the displacement of RNA polymerase is not complete thereby permitting the “reduction of gene expression” of the desired gene.

### (ii) Nucleic Acid-Guided Endonucleases

Different types of nucleic acid-guided endonucleases can be used in the compositions and methods of the invention to facilitate gene editing. Exemplary, nonlimiting, types of nucleic acid-guided endonucleases suited for the compositions and methods of the invention include RNA-guided endonucleases, DNA-guided endonucleases, and single-base editors.

In some embodiments, the nuclease can be an RNA-guided endonuclease. As used herein, the term “RUNA-guided endonuclease” refers to an endonuclease that forms a complex with an RNA molecule that comprises a region complementary to a selected target DNA sequence, such that the RNA molecule binds to the selected sequence to direct endonuclease activity to the selected target DNA sequence.

In one embodiment, the RNA-guided endonuclease is a CRISPR enzyme, as discussed herein. In some embodiments, the RNA-guided endonuclease comprises nickase activity. In some embodiments, the RNA-guided endonuclease directs cleavage of one or both strands at the location of a target sequence, such as within the target sequence and/or within the complement of the target sequence. In some embodiments, the RNA-guided endonuclease directs cleavage of one or both strands within about 1, 2, 3, 4, 5, 6, 7, 8, 9, 10, 15, 20, 25, 50, 100, 200, 500, or more base pairs from the first or last nucleotide of a target sequence. In other embodiments, the nickase activity is directed to one or more sequences on the cleavable ceDNA vectors themselves, for example, to loosen the sequence constraint such that the HDR template is exposed for HDR interaction with the genomic sequence of the target gene.

In certain embodiments, it is contemplated that the nickase cuts at least 1 site, at least 2 sites, at least 3 sites, at least 4 sites, at least 5 sites, at least 6 sites, at least 7 sites, at least 8 sites, at least 9 sites, at least 10 sites or more on the desired nucleic acid sequence (e.g., one or more regions of the cleavable ceDNA vector). In another embodiment, it is contemplated that the nickase cuts at 1 and/or 2 sites via trans-nicking. Trans-nicking can enhance genomic editing by HDR, which is high-fidelity, introduces fewer errors, and thus reduces unwanted off-target effects.

In some embodiments, an expression construct or vector encodes an RNA-guided endonuclease that is mutated with respect to a corresponding wild-type enzyme such that the mutated endonuclease lacks the ability to cleave one strand of a target polynucleotide containing a target sequence.

In some embodiments, the nucleic acid sequence encoding the RNA-guided endonuclease is codon optimized for expression in particular cells, such as eukaryotic cells. The eukaryotic cells can be derived from a particular organism, such as a mammal. Non-limiting examples of mammals can include human, mouse, rat, rabbit, dog, or non-human primate. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence.

In some embodiments, the RNA-guided endonuclease is part of a fusion protein comprising one or more heterologous protein domains (e.g., about or more than about 1, 2, 3, 4, 5, 6, 7, 8, 9, 10, or more domains in addition to the endonuclease). An RNA-guided endonuclease fusion protein can comprise any additional protein sequence, and optionally a linker sequence between any two domains. Examples of protein domains that can be fused to an RNA-guided endonuclease include, without limitation, epitope tags, reporter gene sequences, purification tags, fluorescent proteins and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, VS tags, FLAG tags, influenza hemagglutinin (HA) tags, Myc tags, VSV-G tags, glutathione-S-transferase (GST), chitin binding protein (CBP), maltose binding protein (MBP), poly(NANP), tandem affinity purification (TAP) tag, myc, AcV5, AU1, AUS, E, ECS, E2, nus, Softag 1, Softag 3. Strep, SBP, Glu-Glu, HSV, KT3, S, SI, T7, biotin carboxyl carrier protein (BCCP), calmodulin, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, beta-glucuronidase, luciferase, green fluorescent proteins (e.g., GFP, GFP-2, tagGFP, turboGFP, sfGFP, EGFP, Emerald, Azami Green, Monomeric Azami Green, CopGFP, AceGFP, ZsGreen1), HcRed, DsRed, cyan fluorescent protein (CFP), yellow fluorescent proteins (e.g., YFP, EYFP, Citrine, Venus YPet, PhiYFP, ZsYellow1), cyan fluorescent proteins (e.g., ECFP, Cerulean, CyPet AmCyan1, Midoriishi-Cyan) red fluorescent proteins (e.g., mKate, mKate2, mPlum, DsRed monomer, mCherry, mRFP1, DsRed-Express, DsRed2, HcRed-Tandem, HcRed1, AsRed2, eqFP611, mRaspberry, mStrawberry, Jred), orange fluorescent proteins (e.g., mOrange, mKO, Kusabira-Orange, monomeric Kusabira-Orange, mTangerine, tdTomato) and autofluorescent proteins including blue fluorescent protein (BFP). An RNA-guided endonuclease can be fused to a gene sequence encoding a protein or a fragment of a protein that binds DNA molecules or binds to other cellular molecules, including but not limited to maltose binding protein (MBP), S-tag, Lex A DNA binding domain (DBD) fusions, GAL4 DNA binding domain fusions, and herpes simplex virus (HSV) BP16 protein fusions. In some embodiments, a tagged endonuclease is used to identify the location of a target sequence.

It is contemplated herein that at least two (e.g., at least 3, at least 4, at least 5, at least 6, at least 7, at least 8, at least 9, at least 10, at least 12, at least 15 or more) different Cas enzymes are administered or are in contact with a cell at substantially the same time. Any combination of double-stranded break-inducing Cas enzymes, Cas nickases, catalytically inactive Cas enzymes (e.g., dCas9), modified Cas enzymes, truncated Cas9, etc. are contemplated for use in combination with the methods and compositions described herein.

(iii) CRISPR/Cas Systems

As known in the art, a CRISPR-CAS9 system is a particular set of nucleic-acid guided-nuclease-based systems that includes a combination of protein and ribonucleic acid (“RNA”) that can alter the genetic sequence of an organism. The CRISPR-CAS9 system continues to develop as a powerful tool to modify specific deoxyribonucleic acid (“DNA”) in the genomes of many organisms such as microbes, fungi, plants, and animals. For example, mouse models of human disease can be developed quickly to study individual genes much faster, and easily change multiple genes in cells at once to study their interactions. One of ordinary skill in the art may select between a number of known CRISPR systems such as Type I, Type II, and Type III. Type 11 CRISPR-CAS system has a well-known mechanism including three components: (1) a crDNA molecule, which is called a “guide sequence” or “targeter-RNA”; (2) a “tracr RNA” or “activator-RNA”; and (3) a protein called Cas9.

To alter the DNA molecule, a number of interactions occur in the system including: (1) the guide sequence binding by specific base pairing to a specific sequence of DNA of interest (“target DNA”), (2) the guide sequence binds by specific base pairing at another sequence to an activator-RNA, and (3) activator-RNA interacts with the Cas protein (e.g., Cas9 protein), which then acts as a nuclease to cut the target DNA at a specific site. Suitable systems for use in accordance with ceDNA vectors in accordance with the present disclosure are further described in Van Nierop, et al. Stimulation of homology-directed gene targeting at an endogenous human locus by a nicking endonuclease, Nucleic Acid Research, August 2009 and Ran et al., Double nicking by RNA-guided CRISPR Cas9 for enhanced genome editing specificity.

ceDNA vectors in accordance with the present disclosure can be designed to include nucleotides encoding one or more components of these systems such as the guide sequence, tracr RNA, or Cas (e.g., Cas9). In certain embodiments, a single promoter drives expression of a guide sequence and tracr RNA, and a separate promoter drives Cas (e.g., Cas9) expression. One of skill in the art will appreciate that certain Cas nucleases require the presence of a protospacer adjacent motif (PAM) adjacent to a target nucleic acid sequence. In some embodiments, the PAM may be adjacent to or within 1, 2, 3, or 4 nucleotides of the 3′ end of the target sequence. The length and the sequence of the PAM can depend on the particular Cas protein.

Exemplary PAM sequences include NGG, NGGNG, NG, NAAAAN, NNAAAAAW, NNNNACA, GNNNCNNA, TTN and NNNNGATT (wherein N is defined as any nucleotide and W is defined as either A or T). In some embodiments, the PAM sequence can be on the guide RNA, for example, when editing RNA.

In some embodiments, RNA-guided nucleases including Cas and Cas9 are suitable for use in ceDNA vectors designed to provide one or more components for genome engineering using the CRISPR-Cas9 system See e.g., US publication 2014/0170753 herein incorporated by reference in its entirety. CRISPR-Cas 9 provides a set of tools for Cas9-mediated genome editing via non-homologous end joining (NHEJ), homology-directed recombination (HDR), or microhomology-mediated end-joining (MMEJ) in mammalian cells, as well as generation of modified cell lines for downstream functional studies. To minimize off-target cleavage, the CRISPR-Cas9 system may include a double-nicking strategy using the Cas9 nickase mutant with paired guide RNAs. This system is known in the art, and described in, for example, Ran et al., Genome engineering using the CRISPR-Cas9 system, Nature Protocols, 24 October 2013, and Zhang, et al., Efficient precise knockin with a double cut HDR donor after CRISPR/Cas9-mediated double-stranded DNA cleavage, Genome Biology, 2017 (both references are herein incorporated by reference in their entirety).

In certain embodiments, the gene editing system includes a nuclease, guide RN As that are directed to a target sequence, and a cleavable or a cleaved ceDNA. For example, a nicking CAS, such as nCAS9 D10A can be used to increase the efficiency of gene editing. The guide RNAs can direct nCAS nicking of the cleavable ceDNA and/or the target sequence, thereby releasing torsional constraints of ceDNA for more efficient gene repair and/or expression. Using a nicking nuclease relieves the torsional constraints while retaining sequence and structural integrity allowing the nicked DNA can persist in the nucleus. The guide RNAs can be directed to the same strand of DNA or the complementary strand. The guide RNAs can be directed to e.g., the ITRS, or sequences proceeding promoters, or homology domains etc.

In one embodiment, the RNA-guided endonuclease is a CRISPR enzyme, such as a Cas protein. Non-limiting examples of Cas proteins include Cas1, Cas1B, Cas2, Cas3, Cas4, Cas5, Cas5e (CasD), Cas6, Cas6e, Cas6f, Cas7, Cas8, Cas8al, Cas8a2, Cas8b, Cas8c, Cas9 (also known as Csn1 and Csx12), Cas10, Cas10d, Cas13, Cas13a, Cas13c, CasF, CasH, Csy1, Csy2, Csy3, Cse1, Cse2, Cse3, Cse4, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx11, Csx16, CsaX, Csz1, Csx3, Csx1, Csx15, Csf1, Csf2, Csf3, Csf4, Cul966, Cpf1, C2c1, C2c3, homologs thereof, or modified versions thereof. In one embodiment, the Cas protein is Cas9. In another embodiment, the Cas protein is nuclease-dead Cas9 (dCas9) or a Cas9 nickase. In one embodiment, the Cas protein is a nicking Cas enzyme (nCas).

Typically, the RNA-guided endonuclease comprises DNA cleavage activity, such as the double strand breaks initiated by Cas9. In some embodiments, the RNA-guided endonuclease is Cas9, for example, Cas9 from S. pyogenes or S. pneumoniae. Other non-limiting bacterial sources of Cas9 include Streptococcus pyogenes, Streptococcus pasteurianus Streptococcus thermophilus, Streptococcus sp., Nocardiopsis dassonvillei, Streptomyces pristinaespiralis, Streptomyces viridochromogenes, Streptosporangium roseum, Streptosporangium roseum, Staphylococcus aureus, Alicyclobaccillus acidocaldarius, Bacillus pseudomycoides, Bacillus selenitireducens, Exiguobacterium sibiricum, Francisella novic ida, Wolinella succinogenes, Lactobacillus delbrueckii, Lactobacillus salivarius, Listeria innocua. Lactobacillus gasseri, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Fibrobacter succinogene, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Sutterella wadsworthensis, Gamma proteobacterium, Neisseria cinerea, Neisseria meningitidis, Campylobacter jejuni, Campylobacter lari, Parvibaculum lavamentivorans, Corynebacterium diphtheria, Pasteurella multocida, Rhodospirillum rubrum, Nocardiopsis dassonvillei, or Acaryochloris marina.

An exemplary S. pyogenes Cas9 protein sequence is shown as SEQ ID NO: 11, (see Deltcheva et al., Nature 471, 602-607 (2011) hereby incorporated by reference in its entirety).

In one embodiment, the Cas9 nickase comprises nCas9 D10A. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. In some embodiments, a Cas9 nickase can be used in combination with guide sequence(s), e.g., two guide sequences, which target respectively sense and antisense strands of the DNA target. This combination allows both strands to be nicked and used to induce non-homologous end joining (NHEJ) repair.

In some embodiments, the RNA-guided endonuclease is Cas13. A catalytically inactive Cas13 (dCas13) can be used to edit mRNA sequences as described in e.g., Cox, D et al. RNA editing with CRISPR-Cas13 Science (2017) DOI: 10.1126/science.aaq0180, which is herein incorporated by reference in its entirety.

In some embodiments, the endonuclease is Cas9, or an amino acid or functional fragment of a nuclease having at least 60%, more preferably at least 65%, more preferably at least 70%, more preferably at least 75%, more preferably at least 80%, more preferably at least 85%, even more preferably at least 90%, and most preferably at least 95% sequence identity to SEQ ID NO:12 (Cas9) or consisting of SEQ ID NO:12.

In certain embodiments, Cas9 includes one or more mutations in a catalytic domain rendering the Cas9 a nickase that cleaves a single DNA strand, such as those described in U.S. Patent Publication No. 2017-0191078-A9 (incorporated by reference in its entirety).

In some embodiments, the cleavable ceDNA vectors of the present disclosure are suitable for use in systems and methods based on RNA-programmed Cas9 having gene-targeting and genome editing functionality. For example, the cleavable ceDNA vectors of the present disclosure are suitable for use with Clustered Regularly Interspaced Short Palindromic Repeats or the CRISPR associated (Cas) systems for gene targeting and gene editing. CRISPR Cas9 systems are known in the art and described, e.g., in U.S. patent application Ser. No. 13/842,859 filed on March 2013, and U.S. Pat. Nos. 8,697,359, 8,771,945, 8,795,965, 8,865,406, 8,871,445 all of which are herein incorporated by reference in their entirety.

It is also contemplated herein that Cas9, a Cas9 nickase, or a deactivated Cas9 (dCas9, or also referred to a nuclease dead Cas9 or “catalytically inactive”) are also prepared as fusion proteins with FokI, such that gene editing or gene expression modulation occurs upon formation of FokI heterodimers.

Further, dCas9 can be used to activate (CRISPRa) or inhibit (CRISPRi) expression of a desired gene at the level of regulatory sequences upstream of the target gene sequence. CRISPRa and CRISPRi can be performed, for example, by fusing dCas9 with an effector region (e.g., dCas9/effector fusion) and supplying a guide RNA that directs the dCas9/effector fusion protein to bind to a sequence upstream of the desired or target gene (e.g., in the promoter region). Since dCas9 has no nuclease activity, it remains bound to the target site in the promoter region and the effector portion of the dCas9/effector fusion protein can recruit transcriptional activators or repressors to the promoter site. As such, one can activate or reduce gene expression of a target gene as desired. Previous work in the literature indicates that the use of a plurality of guide RNAs co-expressed with dCas9 can increase expression of a desired gene (see e.g., Maeder et al. CRISPR RNA-guided activation of endogenous human genes Nat Methods 10(10):977-979 (2013). In some embodiments, it is desirable to permit inducible repression of a desired gene. This can be achieved, for example, by using guide RNA binding sites in promoter regions upstream of the transcription start site (see e.g., Gao et al. Complex transcriptional modulation with orthogonal and inducible dCas9 regulators. Nature Methods (2016)). In some embodiments, a nuclease dead version of a DNA endonuclease (e.g., dCas9) can be used to inducibly activate or increase expression of a desired gene, for example, by introduction of an agent that interacts with an effector domain (e.g., a small molecule or at least one guide RNA) of a dCas9/effector fusion protein. In other embodiments, it is also contemplated herein that dCas9 can be fused to a chemical- or light-inducible domain, such that gene expression can be modulated using extrinsic signals. In one embodiment, inhibition of a target gene's expression is performed using dCas9 fused to a KRAB repressor domain, which may be beneficial for improved inhibition of gene expression in mammalian systems and have few off-target effects. Alternatively, transcription-based activation of a gene can be performed using a dCas9 fused to the omega subunit of RNA polymerase, or the transcriptional activators VP64 or p65.

Accordingly, in some embodiments, the methods and compositions described herein, e.g., vectors can comprise and/or be used to deliver CRISPRi (CRISPR interference) and/or CRISPRa (CRISPR activation) systems to a host cell. CRISPRi and CRISPRa systems comprise a deactivated RNA-guided endonuclease (e.g., Cas9) that cannot generate a double strand break (DSB). This permits the endonuclease, in combination with the guide RNAs, to bind specifically to a target sequence in the genome and provide RNA-directed reversible transcriptional control.

In some embodiments of CRISPRi, the endonuclease can comprise a KRAB effector domain. Either with or without the KRAB effector domain, the binding of the deactivated nuclease to the genomic sequence can, e.g., block transcription initiation or progression and/or interfere with the binding of transcriptional machinery or transcription factors.

In CRISPRa, the deactivated endonuclease can be fused with one or more transcriptional activation domains, thereby increasing transcription at or near the site targeted by the endonuclease. In some embodiments, CRISPRa can further comprise gRNAs which recruit further transcriptional activation domains, sgRNA design for CRISPRi and CRISPRa is known in the art (see, e.g., Horlbeck et al. eLife. 5, e19760 (2016); Gilbert et al., Cell. 159, 647-661 (2014); and Zalatan et al., Cell. 160, 339-350 (2015): each of which is incorporated by reference here in its entirety). CRISPRi and CRISPRa-compatible sgRNA can also be obtained commercially for a given target (see, e.g., Dharmacon; Lafayette, CO). Further description of CRISPRi and CRISPRa can be found, e.g., in Qi et al., Cell. 152, 1173-1183 (2013); Gilbert et al., Cell. 154, 442-451 (2013); Cheng et al., Cell Res. 23, 1163-1171 (2013); Tanenbaum et al. Cell. 159, 635-646 (2014): Konermann et al., Nature. 517, 583-588 (2015): Chavez e al., Nat. Methods. 12, 326-328 (2015); Liu et al., Science. 355 (2017); and Goyal et al., Nucleic Acids Res. (2016); each of which is incorporated by reference herein in its entirety.

Accordingly, in some embodiments described herein is a gene editing system comprising a deactivated endonuclease, e.g., RNA-guided endonuclease and/or Cas9, wherein the deactivated endonuclease lacks endonuclease activity, but retains the ability to bind DNA in a site-specific manner, e.g., in combination with one or more guide RNAs and/or sgRNAs. In some embodiments, the vector can further comprise one or more tracrRNAs, guide RNAs, or sgRNAs. In some embodiments, the deactivated endonuclease can further comprise a transcriptional activation domain. In some embodiments, vectors of the present disclosure are also useful for deactivated nuclease systems, such as CRISPRi or CRISPRa dCas systems, nCas, or Cas13 systems, all well known in the art.

It is also contemplated herein that the vectors described herein can be used in combination with dCas9 to visualize genomic loci in living cells (see e.g., Ma et al. Multicolor CRISPR labeling of chromosomal loci in human cells PNAS I 12(10):3002-3007 (2015)). CRISPR mediated visualization of the genome and its organization within the nucleus is also called the 4-D nucleome. In one embodiment, dCas9 is modified to comprise a fluorescent tag. Multiple loci can be labeled in distinct colors, for example, using orthologs that are each fused to a different fluorescent label. This technique can be expanded to study genome structure, for example, by using guide RNAs that bind Alu sequences to aid in mapping the location of guide RNA-specified repeats (see e.g., McCaffrey et al. Nucleic Acids Res 44(2):e11 (2016)). Thus, in some embodiments, mapping of clinically significant loci is contemplated herein, for example, for the identification and/or diagnosis of Huntington's disease, among others. Methods of performing genome visualization or genetic screens with a gene editing system are known in the art and/or are described in, for example, Chen et al. Cell 155:1479-1491 (2013); Singh et al. Nat Commun 7:1-8 (2016); Korkmaz et al. Nat Biotechnol 34:1-10 (2016); Hart et al. Cell 163:1515-1526 (2015); the contents of each of which are incorporated herein by reference in their entirety.

In some embodiments, it may be desirable to edit a single base in the genome, for example, modifying a single nucleotide polymorphism associated with a particular disease (see e.g., Komor, A C et al. Nature 533:420-424 (2016); Nishida, K er al. Targeted nucleotide editing using hybrid prokaryotic and vertebrate adaptive immune systems. Science (2016)). Single nucleotide base editing makes use of base-converting enzyme tethered to a catalytically inactive endonuclease (e.g., nuclease dead Cas9) that does not cut the target gene loci. After the base conversion by a base editing enzyme, the system makes a nick on the opposite, unedited strand, which is repaired by the cell's own DNA repair mechanisms. This results in the replacement of the original nucleotide, which is now a “mismatched nucleotide,” thus completing the conversion of a single nucleotide base pair. Endogenous enzymes are available for effecting the conversion of G/C nucleotide pairs to A/T nucleotide pairs, for example, cytidine deaminase, however there is no endogenous enzyme for catalyzing the reverse conversion of A/T nucleotide pairs to G/C ones. Adenine deaminases (e.g., TadA), that usually only act on RNA to convert adenine to inosine, have been evolutionarily selected for in bacterial systems to identify adenine deaminase mutants that act on DNA to convert adenosine to inosine (see e.g., Gaudelli et al Nature (2017), in press doi:10.1038/nature24644, the contents of which are incorporated by reference in its entirety).

In some embodiments, dCas9 or a modified Cas9 with a nickase function can be fused to an enzyme having a base editing function (e.g., cytidine deaminase APOBEC1 or a mutant TadA). The base editing efficiency can be further improved by including an inhibitor of endogenous base excision repair systems that remove uracil from the genomic DNA. See Gaudelli et al. (2017) programmable base editing of A-T to G-C in genomic DNA without DNA cleavage, Nature Published online 25 Oct. 2017, herein incorporated by reference in its entirety.

It is also contemplated herein that the desired endonuclease is modified by addition of ubiquitin or a polyubiquitin chain. In some embodiments, the ubiquitin can be a ubiquitin-like protein (UBL). Non-limiting examples of ubiquitin-like proteins include small ubiquitin-like modifier (SUMO), ubiquitin cross-reactive protein (UCRP, also known as interferon-stimulated gene 15 (ISG-15)), ubiquitin-related modifier-1 (URM1), neuronal-precursor-cell-expressed developmentally downregulated protein-8 (NEDD8, also called Rubl in S. cerevisiae), human leukocyte antigen F-associated (FAT 10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBL5).

Gene editing systems or compositions thereof can encode for modified DNA endonucleases as described in e.g., Fu et al. Nat Biotechnol 32:279-284 (2013); Ran et al. Cell 154:1380-1389 (2013); Mali et al. Nat Biotechnol 31:833-838 (2013); Guilinger et al. Nat Biotechnol 32:577-582 (2014); Slaymaker et al. Science 351:84-88 (2015); Klenstiver et al. Nature 523:481-485 (2015); Bolukbasi et al. Nat Methods 12:1-9 (2015); Gilbert et al. Cell 154; 442-451 (2012); Anders et al. Mol Cell 61:895-902 (2016); Wright et al. Proc Natl Acad Sci USA 112:2984-2989 (2015); Truong et al. Nucleic Acids Res 43:6450-6458 (2015); the contents of each of which are incorporated herein by reference in their entirety.

### (vi) Multiplex Modulation of Gene Expression and Complex Systems

The lack of size limitations of the cleavable ceDNA vectors as described herein are especially useful in multiplexed editing, CRISPRa or CRISPRi because multiple guide RNAs can be expressed from the same ceDNA vector, if desired. CRISPR is a robust system and the addition of multiple guide RNAs does not substantially alter the efficiency of gene editing, CRISPRa, CRISPRi or CRISPR mediated labeling of nucleic acids. As described elsewhere, the plurality of guide RNAs can be under the control of a single promoter (e.g., a polycistronic transcript) or under the control of a plurality of promoters (e.g., at least 2, at least 3, at least 4, at least 5, at least 6, etc. up to a limit of a 1:1 ratio of guide RNA:promoter sequences).

The multiplex CRISPR/Cas9-Based System takes advantage of the simplicity and low cost of sgRNA design and may be helpful in exploiting advances in high-throughput genomic research using CRISPR/Cas9 technology. For example, the cleavable ceDNA vectors described herein are useful in expressing Cas9 and numerous single guide RNAs (sgRNAs) in difficult cell lines. The multiplex CRISPR/Cas9-Based System may be used in the same ways as the CRISPR/Cas9-Based System described above. Multiplex CRISPR/Cas can be performed as described in Cong, L et al. Science 819 (2013); Wang et al. Cell 153:910-918 (2013); Ma et al. Nat Biotechnol 34:528-530 (2016); the contents of each of which are incorporated herein by reference in their entirety.

In addition to the described transcriptional activation and nuclease functionality, this system will be useful for expressing other novel Cas9-based effectors that control epigenetic modifications for diverse purposes, including interrogation of genome architecture and pathways of endogenous gene regulation. As endogenous gene regulation is a delicate balance between multiple enzymes, multiplexing Cas9 systems with different functionalities will allow for examining the complex interplay among different regulatory signals. The vector described here should be compatible with aptamer-modified gRNAs and orthogonal Cas9s to enable independent genetic manipulations using a single set of gRNAs.

The multiplex CRISPR/Cas9-Based System may be used to activate at least one endogenous gene in a cell. The method includes contacting a cell with the modified lentiviral vector. The endogenous gene may be transiently activated or stably activated. The endogenous gene may be transiently repressed or stably repressed. The fusion protein may be expressed at similar levels to the sgRNAs. The fusion protein may be expressed at different levels compared to the sgRNAs. The cell may be a primary human cell.

The multiplex CRISPR/Cas9-Based System may be used in a method of multiplex gene editing in a cell. The method includes contacting a cell with a cleavable ceDNA vector. The multiplex gene editing may include correcting a mutant gene or inserting a transgene. Correcting a mutant gene may include deleting, rearranging or replacing the mutant gene. Correcting the mutant gene may include nuclease-mediated non-homologous end joining or homology-directed recombination. The multiplex gene editing may include deleting or correcting at least one gene, wherein the gene is an endogenous normal gene or a mutant gene.

The multiplex gene editing may include deleting or correcting at least two genes. For example, at least two genes, at least three genes, at least four genes, at least five genes, at least six genes, at least seven genes, at least eight genes, at least nine genes, or at least ten genes may be deleted or corrected.

The multiplex CRISPR/Cas9-Based System can be used in a method of multiplex modulation of gene expression in a cell. The method includes contacting a cell with the modified lentiviral vector. The method may include modulating the gene expression levels of at least one gene. The gene expression of the at least one target gene is modulated when gene expression levels of the at least one target gene are increased or decreased compared to normal gene expression levels for the at least one target gene. The gene expression levels may be RNA or protein levels.

In some embodiments, it is also contemplated herein that the expression of multiple genes is modulated by introducing multiple, orthogonal Cas with multiple guide RNAs (e.g., multiplex modulation of gene expression or “orthogonal dCas9 systems”). For example, different Cas proteins or Cas9 proteins. One of skill in the art will appreciate that the plurality of guide RNAs should be designed to minimize off-target effects or interaction of the RNAs with one another. Orthogonal dCas9 systems permit the simultaneous activation of certain desired genes with repression of other desired genes. For example, a plurality of orthogonal Cas proteins (e.g., Cas9 proteins) derived from a combination of bacterial species e.g., S. pyogenes, N. meninigitidis, S. thermophilus and T. denticola can be used in combination as described in e.g., Esvelt, K et al. Nature Methods 10(11):1116-1121 (2013), which is herein incorporated by reference in its entirety. In some embodiments, a plurality of nucleic acid sequences encoding a plurality of guide RNAs are present on the same vector. Further, each dCas9 can be paired with a discrete inducible system, which can allow for independent control of activation and/or repression of the desired genes. In addition, this inducible orthogonal dCas9 system can also permit regulation of gene expression in a temporal manner (see e.g., Gao et al. Nature Methods Complex transcriptional modulation with orthogonal and inducible dCas9 regulators (2016)).

### B. Homology-Directed Recombination (HDR) and Microhomology-Mediated End Joining (MMEJ) Repair Templates

In some embodiments, a homology-directed recombination (HDR) or MMEJ template or “repair” template is provided in the cleavable ceDNA vector, e.g., as the donor template comprising the donor sequence. It is contemplated herein that a repair template can be used to repair or modify (e.g., creating a designed mutation) a gene sequence or to insert a new sequence, for example, to manufacture a therapeutic protein. In some embodiments, a repair template is designed to serve as a template in homologous recombination, such as within or near a target sequence nicked or cleaved by a nuclease described herein, e.g., an RNA-guided endonuclease, such as a CRISPR enzyme as a part of a CRISPR complex. A repair or donor template can be of any suitable length, such as about or more than about 10, 15, 20, 25, 50, 75, 100, 150, 200, 500, 1000, 1500, 2000, 2500, 3000, or more bps in length. In some embodiments, the template polynucleotide is complementary to a portion of a polynucleotide comprising a target sequence in the host cell genome. When optimally aligned, a template polynucleotide can overlap with one or more nucleotides of a target sequence (e.g., about or more than about 1, 5, 10, 15, 20, 25, 30, 35, 40, 45, 50, 60, 70, 80, 90, 100, 110, 120, 130, 140, 150, 160, 170, 180, 190, 200, 250, 300, 400, 500, or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence. In one embodiment, the homology arms of the repair template are directional (i.e., not identical and therefore bind to the sequence in a particular orientation). In some embodiments, two or more templates are provided to repair a single gene in a cell, or two different genes in a cell. In some embodiments, multiple copies of at least one template are provided to a cell.

In some embodiments, the template sequence can be substantially identical to a portion of an endogenous target gene sequence but comprises at least one nucleotide change. In some embodiments, the repair of the cleaved target nucleic acid molecule can result in, for example, (i) one or more nucleotide changes in an RNA expressed from the target gene, (ii) altered expression level of the target gene, (iii) gene knockdown, (iv) gene knockout, (v) restored gene function, or (vi) gene knockout and simultaneous insertion of a gene. As will be readily appreciated by one of skill in the art the repair of the cleaved target nucleic acid molecule with the template can result in a change in an exon sequence, an intron sequence, a regulatory sequence, a transcriptional control sequence, a translational control sequence, a splicing site, or a non-coding sequence of the target gene. In other embodiments, the template sequence can comprise an exogenous sequence which can result in a gene-knock-in. Integration of the exogenous sequence can result in a gene knock-out.

In certain embodiments, the donor or repair template is in a capsid-free ceDNA vector also including one or more integration elements such as a 5′ homology arm, and/or a 3 homology arm. At a minimum in certain such embodiments, ceDNA comprises, from 5′ to 3′, a 5′ homology arm, a donor sequence, a 3′ homology arm, and at least one ITR, wherein the at least one ITR is upstream of the 5′ homology arm or downstream of the 3′ homology arm. In certain embodiments, the donor sequence (such as, but not limited to, Factor IX or Factor VIII (or e.g., any other therapeutic protein of interest) is a nucleotide sequence to be inserted into the chromosome of a host cell. In certain embodiments, the donor sequence is not originally present in the host cell or may be foreign to the host cell. In certain embodiments, the donor sequence is an endogenous sequence present at a site other than the predetermined target site. In certain embodiments, the donor sequence is an endogenous sequence similar to that of the pre-determined target site (e.g., replaces an existing erroneous sequence). In certain embodiments, the donor sequence is a sequence endogenous to the host cell, but which is present at a site other than the predetermined target site. In some embodiments, the donor sequence is a coding sequence or non-coding sequence. In some embodiments, the donor sequence is a mutant locus of a gene. In certain embodiments, the donor sequence may be an exogenous gene to be inserted into the chromosome, a modified sequence that replaces the endogenous sequence at the target site, a regulatory element, a tag or a coding sequence encoding a reporter protein and/or RNA. In some embodiments, the donor sequence may be inserted in frame into the coding sequence of a target gene for expression of a fusion protein. In certain embodiments, the donor sequence is not an entire ORF (coding/donor sequence), but just a corrective portion of DNA that is meant to replace a desired target. In certain embodiments, the donor sequence is inserted in-frame behind an endogenous promoter such that the donor sequence is regulated similarly to the naturally-occurring sequence.

In certain embodiments, the donor or repair template may optionally include a promoter therein as described above in order to drive a coding sequence. Such embodiments may further include a poly-A tail within the donor sequence to facilitate expression.

In certain embodiments, the donor or repair template may be a predetermined size, or sized by one of ordinary skill in the art. In certain embodiments, the donor or repair sequence may be at least or about any of 10 base pairs, 15 base pairs, 20 base pairs, 25 base pairs, 50 base pairs, 60 base pairs. 75 base pairs, 100 base pairs, at least 150 base pairs, 200 base pairs, 300 base pairs, 500 base pairs, 800 base pairs, 1000 base pairs, 1,500 base pairs, 2,000 base pairs, 2500 base pairs, 3000 base pairs, 4000 base pairs, 4500 base pairs, and 5,000 base pairs in length or about 1 base pair to about 10 base pairs, or about 10 base pairs to about 50 base pairs, or between about 50 base pairs to about 100 base pairs, or between about 100 base pairs to about 500 base pairs, or between about 500 base pairs to about 5,000 base pairs in length. In certain embodiments, the donor or repair sequence includes only 1 base pair to repair a single mutated nucleotide in the genome.

Non-limiting examples of suitable donor or repair sequence(s) for use in accordance with the present disclosure include a promoter-less coding sequence corresponding to one or more disease-related sequences having at least 60%, more preferably at least 65%, more preferably at least 70%, more preferably at least 75%, more preferably at least 80%, more preferably at least 85%, even more preferably at least 90%, and most preferably at least 95% sequence identity to one of the disease-related molecules described herein. In one embodiment, the coding sequence has at least 60%, more preferably at least 65%, more preferably at least 70%, more preferably at least 75%, more preferably at least 80%, more preferably at least 85%, even more preferably at least 90%, and most preferably at least 95% sequence identity to SEQ ID NO: 825 or a donor sequence consisting of SEQ ID NO: 825. In certain embodiments, such as where the sequence is added rather than replaced, a promoter can be provided.

For integration of the donor or repair template into the host cell genome, the cleavable ceDNA vector may rely on the polynucleotide sequence encoding the donor sequence or any other element of the vector for integration into the genome by homologous recombination such as the 5′ and 3′ homology arms shown therein. For example, the cleavable ceDNA vector may contain nucleotides encoding 5′ and 3′ homology arms for directing integration by homologous recombination into the genome of the host cell at a precise location(s) in the chromosome(s). To increase the likelihood of integration at a precise location, the 5′ and 3′ homology arms may include a sufficient number of nucleic acids, such as about 30 to about 5,000 base pairs, or about 50 to about 5,000 base pairs, or 100 to 5,000 base pairs, or 500 to 5,000 base pairs, which have a high degree of sequence identity or homology to the corresponding target sequence to enhance the probability of homologous recombination. The 5′ and 3′ homology arms may be any sequence that is homologous with the target sequence in the genome of the host cell. Furthermore, the 5′ and 3′ homology arms may be non-encoding or encoding nucleotide sequences. In certain embodiments, the homology between the 5′ homology arm and the corresponding sequence on the chromosome is at least any of 80%, 85%, 90%, 95%, 97%, 98%, 99%, or 100%. In certain embodiments, the homology between the 3′ homology arm and the corresponding sequence on the chromosome is at least any of 80%, 85%, 90%, 95%, 97%, 98%, 99%, or 100%. In certain embodiments, the 5′ and/or 3′ homology arms can be homologous to a sequence immediately upstream and/or downstream of the integration or DNA cleavage site on the chromosome. Alternatively, the 5′ and/or 3′ homology arms can be homologous to a sequence that is distant from the integration or DNA cleavage site, such as at least 1, 2, 5, 10, 15, 20, 25, 30, 50, 100, 200, 300, 400, or 500 bp away from the integration or DNA cleavage site, or partially or completely overlapping with the DNA cleavage site. In certain embodiments, the 3′ homology arm of the nucleotide sequence is proximal to the altered ITR.

In certain embodiments, the efficiency of integration of the donor sequence is improved by extraction of the transgene cassette comprising the donor sequence from the cleavable ceDNA vector prior to integration. In one nonlimiting example, gRNA target sequences (TS) and PAM sequences may be engineered 5′ to the 5′ homology arm, 3′ to the 3′ homology arm, or both. If such a gRNA target sequence is present with respect to both homology arms, then the site may be the same or different between the two homology arms. When the cleavable ceDNA vector is cleaved by the nuclease guided by the gRNA, the resulting cleaved ceDNA comprises the 5′ homology arm-donor sequence-3′ homology arm, and can be more readily recombined with the desired genomic locus. It will be appreciated by one of ordinary skill in the art that this cleaved ceDNA may additionally comprise other elements such as, but not limited to, one or more of the following: a regulatory region, a nuclease, and an additional donor sequence. In certain aspects, the nuclease and/or gRNA is encoded on a second ceDNA vector which is separately delivered. In certain aspects, the nuclease and/or gRNA is introduced to the nucleus by a non-ceDNA-based means of delivery. In certain embodiments, the nuclease and/or gRNA are introduced after the cleavable ceDNA is delivered to the nucleus. In certain embodiments, the nuclease, at least one gRNA, and the cleavable ceDNA vector or cleaved ceDNA are transported to the nucleus simultaneously. In certain embodiments, the nuclease and/or gRNA are already present upon introduction of the cleavable ceDNA or cleaved ceDNA.

In certain embodiments, the donor sequence is foreign to the 5′ homology arm or 3′ homology arm. In certain embodiments, the donor sequence is not endogenously found between the sequences comprising the 5′ homology arm and 3′ homology arm. In certain embodiments, the donor sequence is not endogenous to the native sequence comprising the 5′ homology arm or the 3′ homology arm. In certain embodiments, the 5′ homology arm is homologous to a nucleotide sequence upstream of a nuclease cleavage site on a chromosome. In certain embodiments, the 3′ homology arm is homologous to a nucleotide sequence downstream of a nuclease cleavage site on a chromosome. In certain embodiments, the 5′ homology arm or the 3′ homology arm are proximal to the at least one altered ITR. In certain embodiments, the 5′ homology arm or the 3′ homology arm are about 10 to 2000 bp. In certain embodiments, the 5′ homology arm or the 3′ homology arm are about 150 to 2000 bp. In certain embodiments, the 5′ homology arm or the 3′ homology arm are about 50 to 2000 bp. In certain embodiments, the 5′ homology arm or the 3′ homology arm are about 25 to 2000 bp. In certain embodiments, the 5′ homology arm or the 3′ homology arm are about 10 to 2000 bp. In certain embodiments, the homology arms are about 100 bp to about 2000 hp in length that facilitate HDR double-stranded break repair. In certain other embodiments, the homology arms are about I bp to about 100 bp in length (microhomology arms) that facilitate MMEJ double-stranded break repair.

In one embodiment, ceDNA vectors that comprise 5′- and 3′homology arms flanking a donor sequence, as described herein, can be administered in conjunction with another vector (e.g., an additional ceDNA vector, a lentiviral vector, a viral vector, or a plasmid) that encodes a nuclease or a Cas nickase (nCas; e.g., Cas9 nickase). It is contemplated herein that such an nCas enzyme is used in conjunction with a guide RNA that comprises homology to a cleavable ceDNA vector as described herein and can be used, for example, to release physically constrained sequences or to provide torsional release. Releasing physically constrained sequences can, for example, “unwind” the cleavable ceDNA vector such that a homology directed recombination (HDR) or microhomology-mediated end joining (MMEJ) template homology arm(s) within the cleavable ceDNA vector are exposed for interaction with the genomic sequence. In addition, it is contemplated herein that such a system can be used to deactivate ceDNA vectors, if necessary. It will be understood by one of skill in the art that a Cas enzyme that induces a double-stranded break in the cleavable ceDNA vector would be a stronger deactivator of such ceDNA vectors. In one embodiment, the guide RNA comprises homology to a sequence inserted into the cleavable ceDNA vector. In another embodiment, the guide RNA comprises homology to an inverted terminal repeat (ITR) or the homology/insertion elements of the cleavable ceDNA vector. In some embodiments, a cleavable ceDNA vector as described herein comprises an ITR on each of the 5′ and 3′ ends, thus a guide RNA with homology to the ITRs will produce nicking of the one or more ITRs substantially equally. In some embodiments, a guide RNA has homology to some portion of the cleavable ceDNA vector and the donor sequence or template (e.g., to assist with unwinding the cleavable ceDNA vector). It is also contemplated herein that there are certain sites on the cleavable ceDNA vectors that when nicked may result in the inability of the cleavable ceDNA vector to be retained in the nucleus. One of ordinary skill in the art can readily identify such sequences and can thus avoid engineering guide RNAs to such sequence regions. Alternatively, modifying the subcellular localization of a cleavable ceDNA vector to a region outside the nucleus by using a guide RNA that nicks sequences responsible for nuclear localization can be used as a method of deactivating the cleavable ceDNA vector, if necessary or desired.

C. Guide RNAs (gRNAs)

In general, a guide sequence is any polynucleotide sequence having sufficient complementarity with a target polynucleotide sequence to hybridize with the target sequence and direct sequence-specific targeting of an RNA-guided endonuclease complex to the selected genomic target sequence. In some embodiments, a guide RNA binds and e.g., a Cas protein can form a ribonucleoprotein (RNP), for example, a CRISPR/Cas complex.

In some embodiments, the guide RNA (gRNA) sequence comprises a targeting sequence that directs the gRNA sequence to a desired site in the genome, fused to a crRNA and/or tracrRNA sequence that permit association of the guide sequence with the RNA-guided endonuclease. In some embodiments, the degree of complementarity between a guide sequence and its corresponding target sequence, when optimally aligned using a suitable alignment algorithm, is at least 50%, 60%, 75%, 80%, 85%, 90%, 95%. 97.5%, 99%, or more. Optimal alignment can be determined with the use of any suitable algorithm for aligning sequences, such as the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP, and Maq. In some embodiments, a guide sequence is 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. It is contemplated herein that the targeting sequence of the guide RNA and the target sequence on the target nucleic acid molecule can comprise 1, 2, 3, 4, 5, 6, 7, 8, 9, or 10 mismatches. In some embodiments, the guide RNA sequence comprises a palindromic sequence, for example, the self-targeting sequence comprises a palindrome. The targeting sequence of the guide RNA is typically 19-21 base pairs long and directly precedes the hairpin that binds the entire guide RNA (targeting sequence+hairpin) to a Cas such as Cas9. Where a palindromic sequence is employed as the self-targeting sequence of the guide RNA, the inverted repeat element can be e.g., 9, 10, 11, 12, or more nucleotides in length. Where the targeting sequence of the guide RNA is most often 19-21 bp, a palindromic inverted repeat element of 9 or 10 nucleotides provides a targeting sequence of desirable length. The Cas9-guide RNA hairpin complex can then recognize and cut any nucleotide sequence (DNA or RNA) e.g., a DNA sequence that matches the 19-21 base pair sequence and is followed by a “PAM” sequence e.g., NGG or NGA, or other PAM.

The ability of a guide sequence to direct sequence-specific binding of an RNA-guided endonuclease complex to a target sequence can be assessed by any suitable assay. For example, the components of an RNA-guided endonuclease system sufficient to form an RNA-guided endonuclease complex can be provided to a host cell having the corresponding target sequence, such as by transfection with vectors encoding the components of the RNA-guided endonuclease sequence, followed by an assessment of preferential cleavage within the target sequence, such as by Surveyor assay (Transgenomic™, New Haven, CT). Similarly, cleavage of a target polynucleotide sequence can be evaluated in a test tube by providing the target sequence, components of an RNA-guided endonuclease complex, including the guide sequence to be tested and a control guide sequence different from the test guide sequence, and comparing binding or rate of cleavage at the target sequence between the test and control guide sequence reactions. One of ordinary skill in the art will appreciate that other assays can also be used to test gRNA sequences.

A guide sequence can be selected to target any target sequence. In some embodiments, the target sequence is a sequence within a genome of a cell. In some embodiments, the target sequence is the sequence encoding a first guide RNA in a self-cloning plasmid, as described herein. Typically, the target sequence in the genome will include a protospacer adjacent (PAM) sequence for binding of the RNA-guided endonuclease. It will be appreciated by one of skill in the art that the PAM sequence and the RNA-guided endonuclease should be selected from the same (bacterial) species to permit proper association of the endonuclease with the targeting sequence. For example, the PAM sequence for CAS9 is different than the PAM sequence for cpF1. Design is based on the appropriate PAM sequence. To prevent degradation of the guide RNA, the sequence of the guide RNA should not contain the PAM sequence. In some embodiments, the length of the targeting sequence in the guide RNA is 12 nucleotides; in other embodiments, the length of the targeting sequence in the guide RNA is 13, 14, 15, 16, 17, 18, 19, 20, 21, 22, 23, 24, 25, 26, 27, 28, 29, 30, 35 or 40 nucleotides. The guide RNA can be complementary to either strand of the targeted DNA sequence. In some embodiments, when modifying the genome to include an insertion or deletion, the gRNA can be targeted closer to the N-terminus of a protein coding region.

It will be appreciated by one of skill in the art that for the purposes of targeted cleavage by an RNA-guided endonuclease, target sequences that are unique in the genome are preferred over target sequences that occur more than once in the genome. Bioinformatics software can be used to predict and minimize off-target effects of a guide RNA (see e.g., Naito et al. “CRISPRdirect: software for designing CRISPR/Cas guide RNA with reduced off-target sites” Bioinformatics (2014), epub: Heigwer, F., et al. “E-CRISP: fast CRISPR target site identification” Nat. Methods 11, 122-123 (2014); Bae et al. “Cas-OFFinder: a fast and versatile algorithm that searches for potential off-target sites of Cas9 RNA-guided endonucleases” Bioinformatics 30(10):1473-1475 (2014); Aach et al. “CasFinder: Flexible algorithm for identifying specific Cas9 targets in genomes” BioRxiv (2014), among others).

For the S. pyogenes Cas9, a unique target sequence in a genome can include a Cas9 target site of the form MMMMMMMMNNNNNNNNNNNXGG where NNNNNNNNNNNNXGG (N is A, G, T, or C; and X can be any nucleotide) has a single occurrence in the genome. A unique target sequence in a genome can include an S. pyogenes Cas9 target site of the form MMMMMMMMMNNNNNNNNNNNXGG where NNNNNNNNNNNXGG (N is A, G, T, or C; and X can be any nucleotide) has a single occurrence in the genome. For the S. thermophilus CRISPR1 Cas9, a unique target sequence in a genome can include a Cas9 target site of the form MMMMMMMMNNNNNNNNNNNNXXAGAAW where NNNNNNNNNNNNXXAGAAW (N is A, G, T, or C; X can be any nucleotide; and W is A or T) has a single occurrence in the genome. A unique target sequence in a genome can include an S. thermophilus CRISPR 1 Cas9 target site of the form MMMMMMMMMNNNNNNNNNNNXXAGAAW where NNNNNNNNNNNXXAGAAW (N is A, G, T, or C; X can be any nucleotide; and W is A or T) has a single occurrence in the genome. For the S. pyogenes Cas9, a unique target sequence in a genome can include a Cas9 target site of the form MMMMMMMMNNNNNNNNNNNNXGGXG where NNNNNNNNNNNNXGGXG (N is A, G, T, or C; and X can be any nucleotide) has a single occurrence in the genome. A unique target sequence in a genome can include an S. pyogenes Cas9 target site of the form MMMMMMMMMNNNNNNNNNNNXGGXG where NNNNNNNNNNNXGGXG (N is A, G, T, or C; and X can be any nucleotide) has a single occurrence in the genome. In each of these sequences “M” may be A, G, T, or C, and need not be considered in identifying a sequence as unique.

In general, a “crRNA/tracrRNA fusion sequence,” as that term is used herein refers to a nucleic acid sequence that is fused to a unique targeting sequence and that functions to permit formation of a complex comprising the guide RNA and the RNA-guided endonuclease. Such sequences can be modeled after CRISPR RNA (crRNA) sequences in prokaryotes, which comprise (i) a variable sequence termed a “protospacer” that corresponds to the target sequence as described herein, and (ii) a CRISPR repeat. Similarly, the tracrRNA (“transactivating CRISPR RNA”) portion of the fusion can be designed to comprise a secondary structure similar to the tracrRNA sequences in prokaryotes (e.g., a hairpin), to permit formation of the endonuclease complex. In some embodiments, the fusion has sufficient complementarity with a tracrRNA sequence to promote one or more of: (1) excision of a guide sequence flanked by tracrRNA sequences in a cell containing the corresponding tracr sequence; and (2) formation of an endonuclease complex at a target sequence, wherein the complex comprises the crRNA sequence hybridized to the tracrRNA sequence. In general, degree of complementarity is with reference to the optimal alignment of the crRNA sequence and tracrRNA sequence, along the length of the shorter of the two sequences. Optimal alignment can be determined by any suitable alignment algorithm, and can further account for secondary structures, such as self-complementarity within either the tracrRNA sequence or crRNA sequence. In some embodiments, the degree of complementarity between the tracrRUNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracrRNA sequence is at least 5, 6, 7, 8, 9, 10, 11, 12, 13, 14, 15, 16, 17, 18, 19, 20, 25, 30, 40, 50, 60, 70, 80, 90, 100, or more nucleotides in length (e.g., 70-80, 70-75, 75-80 nucleotides in length). In one embodiment, the crRNA is less than 60, less than 50, less than 40, less than 30, or less than 20 nucleotides in length. In other embodiments, the crRNA is 30-50 nucleotides in length; in other embodiments the crRNA is 30-50, 35-50, 40-50,40-45, 45-50 or 50-55 nucleotides in length. In some embodiments, the crRNA sequence and tracrRNA sequence are contained within a single transcript, such that hybridization between the two produces a transcript having a secondary structure, such as a hairpin. In some embodiments, the loop forming sequences for use in hairpin structures are four nucleotides in length, for example, the sequence GAAA. However, longer or shorter loop sequences can be used, as can alternative sequences. The sequences preferably include a nucleotide triplet (for example. AAA), and an additional nucleotide (for example C or G). Examples of loop forming sequences include CAAA and AAAG. In one embodiment, the transcript or transcribed gRNA sequence comprises at least one hairpin. In one embodiment, the transcript or transcribed polynucleotide sequence has at least two or more hairpins. In other embodiments, the transcript has two, three, four or five hairpins. In a further embodiment, the transcript has at most five hairpins. In some embodiments, the single transcript further includes a transcription termination sequence, such as a polyT sequence, for example six T nucleotides. Non-limiting examples of single polynucleotides comprising a guide sequence, a crRNA sequence, and a tracr sequence are as follows (listed 5′ to 3′), where “N” represents a base of a guide sequence, the first block of lower case letters represent the crRNA sequence, and the second block of lower case letters represent the tracr sequence, and the final poly-T sequence represents the transcription terminator:

(i)
(SEQ ID NO: 146)
NNNNNNNNNNNNNNNNNNNNgtttttgtactctcaagatttaGAAAtaaatcttgcagaagctacaaagataagg
ctt catgccgaaatcaacaccctgtcattttatggcagggtgttttcgttatttaaTTTTTT;
(ii)
(SEQ ID NO: 147)
NNNNNNNNNNNNNNNNNNNNgtttttgtactctcaGAAAthcagaagctacaaagataaggcttcatgccgaa
atca acaccctgtcattttatggcagggtgttttcgttatttaaTTTTTT;
(iii)
(SEQ ID NO: 148)
NNNNNNNNNNNNNNNNNNNNgtttttgtactctcaGAAAtgcagaagctacaaagataaggcttcatgccgaa
atca acaccctgtcattttatggcagggtgtTTTTTT;
(iv)
(SEQ ID NO: 149)
NNNNNNNNNNNNNNNNNNNNgttttagagctaGAAAtagcaagttaaaataaggctagtccgttatcaacttga
aaa agtggcaccgagtcggtgcTTTTTT;
(v)
(SEQ ID NO: 150)
NNNNNNNNNNNNNNNNNNNNgttttagagctaGAAATAGcaagttaaaataaggctagtccgttatcaactt
gaa aaagtTTTTTTT;
and
(vi)
(SEQ ID NO: 151)
NNNNNNNNNNNNNNNNNNNNgttttagagctagAAATAGcaagttaaaataaggctagtccgttatcaTTT
TT TTT.

In some embodiments, sequences (i) to (iii) are used in combination with Cas9 from S. thermophilus CRISPR1. In some embodiments, sequences (iv) to (vi) are used in combination with Cas9 from S. pyogenes. In some embodiments, the tracrRNA sequence is a separate transcript from a transcript comprising the crRNA sequence.

In some embodiments, a guide RNA can comprise two RNA molecules and is referred to herein as a “dual guide RNA” or “dgRNA.” In some embodiments, the dgRNA may comprise a first RNA molecule comprising a crRNA, and a second RNA molecule comprising a tracrRNA. The first and second RNA molecules may form an RNA duplex via the base pairing between the flagpole on the crRNA and the tracrRNA. When using a dgRNA, the flagpole need not have an upper limit with respect to length.

In other embodiments, a guide RNA can comprise a single RNA molecule and is referred to herein as a “single guide RNA” or “sgRNA.” In some embodiments, the sgRNA can comprise a crRNA covalently linked to a tracrRNA. In some embodiments, the crRNA and tracrRNA can be covalently linked via a linker. In some embodiments, the sgRNA can comprise a stem-loop structure via the base-pairing between the flagpole on the crRNA and the tracrRNA. In some embodiments, a single-guide RNA is at least 50, at least 60, at least 70, at least 80, at least 90, at least 100, at least 110, at least 120 or more nucleotides in length (e.g., 75-120, 75-110, 75-100, 75-90, 75-80, 80-120, 80-110, 80-100,80-90, 85-120, 85-110, 85-100, 85-90, 90-120, 90-110.90-100, 100-120, 100-120 nucleotides in length). In some embodiments, a gene editing system, vector or composition thereof comprises a nucleic acid that encodes at least 1 gRNA. For example, the second polynucleotide sequence may encode at least 1 gRNA, at least 2 gRNAs, at least 3 gRNAs, at least 4 gRNAs, at least 5 gRNAs, at least 6 gRNAs, at least 7 gRNAs, at least 8 gRNAs, at least 9 gRNAs, at least 10 gRNAs, at least 11 gRNA, at least 12 gRNAs, at least 13 gRNAs, at least 14 gRNAs, at least 15 gRNAs, at least 16 gRNAs, at least 17 gRNAs, at least 18 gRNAs, at least 19 gRNAs, at least 20 gRNAs, at least 25 gRNA, at least 30 gRNAs, at least 35 gRNAs, at least 40 gRNAs, at least 45 gRNAs, or at least 50 gRNAs. The second polynucleotide sequence may encode between 1 gRNA and 50 gRNAs, between 1 gRNA and 45 gRNAs, between 1 gRNA and 40 gRNAs, between 1 gRNA and 35 gRNAs, between 1 gRNA and 30 gRNAs, between 1 gRNA and 25 different gRNAs, between 1 gRNA and 20 gRNAs, between 1 gRNA and 16 gRNAs, between I gRNA and 8 different gRNAs, between 4 different gRNAs and 50 different gRNAs, between 4 different gRNAs and 45 different gRNAs, between 4 different gRNAs and 40 different gRNAs, between 4 different gRNAs and 35 different gRNAs, between 4 different gRNAs and 30 different gRNAs, between 4 different gRNAs and 25 different gRNAs, between 4 different gRNAs and 20 different gRNAs, between 4 different gRNAs and 16 different gRNAs, between 4 different gRNAs and 8 different gRNAs, between 8 different gRNAs and 50 different gRNAs, between 8 different gRNAs and 45 different gRNAs, between 8 different gRNAs and 40 different gRNAs, between 8 different gRNAs and 35 different gRNAs, between 8 different gRNAs and 30 different gRNAs, between 8 different gRNAs and 25 different gRNAs, between 8 different gRNAs and 20 different gRNAs, between 8 different gRNAs and 16 different gRNAs, between 16 different gRNAs and 50 different gRNAs, between 16 different gRNAs and 45 different gRNAs, between 16 different gRNAs and 40 different gRNAs, between 16 different gRNAs and 35 different gRNAs, between 16 different gRNAs and 30 different gRNAs, between 16 different gRNAs and 25 different gRNAs, or between 16 different gRNAs and 20 different gRNAs. Each of the polynucleotide sequences encoding the different gRNAs may be operably linked to a promoter. The promoters that are operably linked to the different gRNAs may be the same promoter. The promoters that are operably linked to the different gRNAs may be different promoters. The promoter may be a constitutive promoter, an inducible promoter, a repressible promoter, or a regulatable promoter.

Modified nucleosides or nucleotides can be present in a guide RNA or mRNA as described herein. An mRNA encoding a guide RNA or a DNA endonuclease (e.g., an RNA-guided nuclease) can comprise one or more modified nucleosides or nucleotides: such mRNAs are called “modified” to describe the presence of one or more non-naturally and/or naturally occurring components or configurations that are used instead of or in addition to the canonical A, G. C, and U residues. In some embodiments, a modified RNA is synthesized with a non-canonical nucleoside or nucleotide, here called “modified.” Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the Y end or 5′ end of the oligonucleotide, e.g., removal, modification or replacement of a terminal phosphate group or conjugation of a moiety, cap or linker (such 3 or 5′ cap modifications may comprise a sugar and/or backbone modification); and (vii) modification or replacement of the sugar (an exemplary sugar modification). Unmodified nucleic acids can be prone to degradation by, e.g., cellular nucleases. For example, nucleases can hydrolyze nucleic acid phosphodiester bonds. Accordingly, in one aspect the guide RNAs described herein can contain one or more modified nucleosides or nucleotides, e.g., to introduce stability toward nucleases. In certain embodiments, the mRNAs described herein can contain one or more modified nucleosides or nucleotides, e.g., to introduce stability toward nucleases. In one embodiment, the modification includes 2′-O-methyl nucleotides. In other embodiments, the modification comprises phosphorothioate (PS) linkages.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens. The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxy methyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxynethylimino.

Modified nucleosides and nucleotides can include one or more modifications to the sugar group, i.e., at sugar modification. For example, the 2′ hydroxyl group (OH) can be modified, e.g., replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion. Examples of 2′ hydroxyl group modifications can include alkoxy or aryloxy (OR, wherein “R” can be, e.g., alkyl, cycloalkyl, aryl, aralkyl, heteroaryl or a sugar); poly ethylene glycols (PEG), 0(CH2CH20)nCH2CH2OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-0-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (L2NA) in which the 2′ hydroxyl can be connected, e.g., by a Ci-6 alkylene or Ci-6 heteroalkylene bridge, to the 4′ carbon of the same ribose sugar, where exemplary bridges can include methylene, propylene, ether, or amino bridges; O-amino (wherein amino can be, e.g., NH2; alkylamino, dialkylamino, heterocyclyl, arylamino, diarylamino, heteroarylamino, or diheteroarylamino, ethylenediamine, or polyamino) and aminoalkoxy, 0(CH2)n-amino, (wherein amino can be, e.g., NH2; alkylamino, dialkylamino, heterocyclyl, arylamino, diarylamino, heteroarylamino, or diheteroarylamino, ethylenediamine, or polyamino). In some embodiments, the 2′ hydroxyl group modification can include “unlocked” nucleic acids (UNA) in which the ribose ring lacks the C2′-C3′ bond. In some embodiments, the 2′ hydroxyl group modification can include the methoxyethyl group (MOE), (OCH2CH2OCH3, e.g., a PEG derivative).

The term “Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., —NH2, alkylamino, dialkylamino, heterocyclyl, arylamino, diarylamino, heteroarylamino, diheteroarylamino, or amino acid); NH(CH2CH2NH)nCH2CH2-amino (wherein amino can be, e.g., as described herein), —NHC(0)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein. The sugar modification can comprise a sugar group which can also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g., L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA and/or tracr RNA. In certain embodiments comprising an sgRNA, one or more residues at one or both ends of the sgRNA may be chemically modified, or the entire sgRNA may be chemically modified. Certain embodiments comprise a 5′ end modification. Certain embodiments comprise a 3′ end modification. In certain embodiments, one or more or all of the nucleotides in single stranded overhang of a guide RNA molecule are deoxynucleotides. The modified mRNA can contain 5′ end and/or 3′ end modifications.

### D. Regulatory Elements

The cleavable ceDNA vectors as described herein can be produced from expression constructs that further comprise a specific combination of cis-regulatory elements. The cis-regulatory elements include, but are not limited to, a promoter, a riboswitch, an insulator, a mir-regulatable element, a post-transcriptional regulatory element, a tissue- and cell type-specific promoter and an enhancer. In some embodiments, the ITR can act as the promoter for the transgene. In some embodiments, the cleavable ceDNA vector comprises additional components to regulate expression of the transgene, for example, regulatory switches as described herein, to regulate the expression of the transgene, or a kill switch, which can kill a cell comprising the cleavable ceDNA vector. Regulatory elements, including Regulatory Switches that can be used in the present invention are more fully discussed in PCT/US18/49996, which is incorporated herein in its entirety by reference.

In embodiments, the second nucleotide sequence includes a regulatory sequence, and a nucleotide sequence encoding a nuclease. In certain embodiments the gene regulatory sequence is operably linked to the nucleotide sequence encoding the nuclease. In certain embodiments, the regulatory sequence is suitable for controlling the expression of the nuclease in a host cell. In certain embodiments, the regulatory sequence includes a suitable promoter sequence, being able to direct transcription of a gene operably linked to the promoter sequence, such as a nucleotide sequence encoding the nuclease(s) of the present disclosure. In certain embodiments, the second nucleotide sequence includes an intron sequence linked to the 5′ terminus of the nucleotide sequence encoding the nuclease. In certain embodiments, an enhancer sequence is provided upstream of the promoter to increase the efficacy of the promoter. In certain embodiments, the regulatory sequence includes an enhancer and a promoter, wherein the second nucleotide sequence includes an intron sequence upstream of the nucleotide sequence encoding a nuclease, wherein the intron includes one or more nuclease cleavage site(s), and wherein the promoter is operably linked to the nucleotide sequence encoding the nuclease.

The cleavable ceDNA vectors can be produced from expression constructs that further comprise a specific combination of cis-regulatory elements such as WHP posttranscriptional regulatory element (WPRE) (e.g., SEQ ID NO: 13) and BGH polyA (SEQ ID NO: 14. Suitable expression cassettes for use in expression constructs are not limited by the packaging constraint imposed by the viral capsid.

### (i). Promoters:

It will be appreciated by one of ordinary skill in the art that promoters used in the ceDNA vectors of the invention should be tailored as appropriate for the specific sequences they are promoting. For example, transgene cassette encoding a guide RNA may not require a promoter at all, since its function is to form a duplex with a specific target sequence on the native DNA to facilitate a recombination event. In contrast, a transgene cassette encoding a nuclease would benefit from a promoter so that it can be efficiently expressed from the vector—and, optionally, in a regulatable fashion.

Expression cassettes of the present invention include a promoter, which can influence overall expression levels as well as cell-specificity. For transgene expression, they can include a highly active virus-derived immediate early promoter. Expression cassettes can contain tissue-specific eukaryotic promoters to limit transgene expression to specific cell types and reduce toxic effects and immune responses resulting from unregulated, ectopic expression. In preferred embodiments, an expression cassette can contain a synthetic regulatory element, such as a CAG promoter (SEQ ID NO: 8). The CAG promoter comprises (i) the cytomegalovirus (CMV) early enhancer element, (ii) the promoter, the first exon and the first intron of chicken beta-actin gene, and (iii) the splice acceptor of the rabbit beta-globin gene. Alternatively, an expression cassette can contain an Alpha-l-antitrypsin (AAT) promoter (SEQ ID NO: 15 or SEQ ID NO: 16), a liver specific (LP1) promoter (SEQ ID NO: 17 or SEQ ID NO: 18), or a Human elongation factor-1 alpha (EF1a) promoter (e.g., SEQ ID NO: 19 or SEQ ID NO: 20). In some embodiments, the expression cassette includes one or more constitutive promoters, for example, a retroviral Rous sarcoma virus (RSV) LTR promoter (optionally with the RSV enhancer), or a cytomegalovirus (CMV) immediate early promoter (optionally with the CMV enhancer, e.g., SEQ ID NO: 21). Alternatively, an inducible promoter, a native promoter for a transgene, a tissue-specific promoter, or various promoters known in the art can be used.

Suitable promoters, including those described above, can be derived from viruses and can therefore be referred to as viral promoters, or they can be derived from any organism, including prokaryotic or eukaryotic organisms. Suitable promoters can be used to drive expression by any RNA polymerase (e.g., pol 1, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6, e.g., SEQ ID NO: 22) (Miyagishi et al., Nature Biotechnology 20, 497-500 (2002)), an enhanced U6 promoter (e.g., Xia et al., Nucleic Acids Res. 2003 Sep. 1; 31(17)), a human HI promoter (Hi) (e.g., SEQ ID NO: 23), a CAG promoter, a human alpha 1-antitypsin (HAAT) promoter (e.g., SEQ ID NO: 24), and the like. In certain embodiments, these promoters are altered at their downstream intron containing end to include one or more nuclease cleavage sites. In certain embodiments, the DNA containing the nuclease cleavage site(s) is foreign to the promoter DNA.

In one embodiment, the promoter used is the native promoter of the gene encoding the therapeutic protein. The promoters and other regulatory sequences for the respective genes encoding the therapeutic proteins are known and have been characterized. The promoter region used may further include one or more additional regulatory sequences (e.g., native), e.g., enhancers, (e.g., SEQ ID NO: 2).

### (ii). Polyadenylation Sequences:

A sequence encoding a polyadenylation sequence can be included in the cleavable ceDNA vector to stabilize an mRNA expressed from the cleavable ceDNA vector, and to aid in nuclear export and translation. In one embodiment, the cleavable ceDNA vector does not include a polyadenylation sequence. In other embodiments, the vector includes at least 1, at least 2, at least 3, at least 4, at least 5, at least 10, at least 15, at least 20, at least 25, at least 30, at least 40, least 45, at least 50 or more adenine dinucleotides. In some embodiments, the polyadenylation sequence comprises about 43 nucleotides, about 40-50 nucleotides, about 40-55 nucleotides, about 45-50 nucleotides, about 35-50 nucleotides, or any range there between.

The expression cassettes can include a poly-adenylation sequence known in the art or a variation thereof, such as a naturally occurring sequence isolated from bovine BGHpA (e.g., SEQ ID NO: 74) or a virus SV40pA (e.g., SEQ ID NO: 10), or a synthetic sequence (e.g., SEQ ID NO: 27). Some expression cassettes can also include SV40 late polyA signal upstream enhancer (USE) sequence. In some embodiments, the, USE can be used in combination with SV40pA or heterologous poly-A signal.

The expression cassettes can also include a post-transcriptional element to increase the expression of a transgene. In some embodiments. Woodchuck Hepatitis Virus (WHP) posttranscriptional regulatory element (WPRE) (e.g., SEQ ID NO: 13) is used to increase the expression of a transgene. Other posttranscriptional processing elements such as the post-transcriptional element from the thymidine kinase gene of herpes simplex virus, or hepatitis B virus (HBV) can be used. Secretory sequences can be linked to the transgenes, e.g., VH-02 and VK-A26 sequences, e.g., SEQ ID NO: 25 (Met Asp Trp Thr Trp Arg Ile Leu Phe Leu Val Ala Ala Ala Thr Gly Ala His Ser) and SEQ ID NO: 26 (Met Leu Pro Ser Gln Leu Ile Gly Phe Leu Leu Leu Trp Val Pro Ala Ser Arg Gly).

(iii). Nuclear Localization Sequences

In some embodiments, the vector encoding an RNA guided endonuclease comprises one or more nuclear localization sequences (NLSs), for example, 1, 2, 3, 4, 5, 6, 7, 8, 9, 10, or more NLSs. In some embodiments, the one or more NLSs are located at or near the amino-terminus, at or near the carboxy-terminus, or a combination of these (e.g., one or more NLS at the amino-terminus and/or one or more NLS at the carboxy terminus). When more than one NLS is present, each can be selected independently of the others, such that a single NLS is present in more than one copy and/or in combination with one or more other NLSs present in one or more copies. Non-limiting examples of NLSs are shown in Table 6.

TABLE 6
Nuclear Localization Signals

| SOURCE | SEQUENCE |
|---|---|
| SV40 virus large T- | PKKKRKV (SEQ ID NO: 152) (encoded by |
| antigen | CCCAAGAAGAAGAGGAAGGTG (SEQ ID NO: 153)) |
| nucleoplasmin | KRPAATKKAGQAKKKK (SEQ ID NO: 154) |
| c-myc | PAAKRVKLD (SEQ ID NO: 155) |
| | RQRRNELKRSP (SEQ ID NO: 156) |
| HRNPA1 M9 | NQSSNFGPMKGGNFGGRSSGPYGGGGQYFAKPRNQGGY |
| | (SEQ ID NO: 157) |
| IBB domain from | RMRIZFKNKGKDTAELRRRRVEVSVELRKAKKDEQILKRRNV |
| importin-alpha | (SEQ ID NO: 158) |
| myoma T protein | VSRKRPRP (SEQ ID NO: 159) |
| | PPKKARED (SEQ ID NO: 160) |
| human p53 | PQPKKKPL (SEQ ID NO: 161) |
| mouse c-abl IV | SALIKKKKKMAP (SEQ ID NO: 162) |
| influenza virus NS1 | DRLRR (SEQ ID NO: 163) |
| | PKQKKRK (SEQ ID NO: 164) |
| Hepatitis virus delta | RKLKKKIKKL (SEQ ID NO: 165) |
| antigen | |
| mouse Mx1 protein | REKKKFLKRR (SEQ ID NO: 166) |
| human poly (ADP- | KRKGDEVDGVDEVAKKKSKK (SEQ ID NO: 167) |
| ribose) polymerase | |
| steroid hormone | RKCLQAGMNLEARKTKK (SEQ ID NO: 168) |
| receptors (human) | |
| glucocorticoid | |

### E. Additional Components of Gene Editing Systems

The compositions and vectors of the present disclosure may contain nucleotides that encode other components for gene editing. For example, to select for specific gene targeting events, a protective shRNA may be embedded in a microRNA and inserted into a ceDNA vector designed to integrate site-specifically into the highly active locus, such as an albumin locus. Such embodiments may provide a system for in vivo selection and expansion of gene-modified hepatocytes in any genetic background such as described in Nygaard et al., A universal system to select gene-modified hepatocytes in vivo, Gene Therapy, Jun. 8, 2016. The cleavable ceDNA vectors of the present disclosure may contain one or more selectable markers that permit selection of transformed, transfected, transduced, or the like cells. A selectable marker is a gene the product of which provides for biocide or viral resistance, resistance to heavy metals, prototrophy to auxotrophs, NeoR, and the like. In certain embodiments, positive selection markers are incorporated into the donor sequences such as NeoR. Negative selections markers may be incorporated downstream the donor sequences, for example a nucleic acid sequence HSV-tk encoding a negative selection marker may be incorporated into a nucleic acid construct downstream the donor sequence. In certain embodiments, a negative selection marker such as HSV TK) and expressing unit that allows to control and select for successful correct site usage, may optionally be positioned outside the homology arms.

In embodiments, the cleavable ceDNA vector of the present disclosure may include a polyadenylation site upstream and proximate to the 5′ homology arm.

In some embodiments, a cleavable ceDNA vector in accordance with the present disclosure is shown including ceDNA specific ITR. The cleavable ceDNA vector includes a Pol III promoter driven (such as U6 and H1) sgRNA expressing unit with optional orientation with respect to the transcription direction. An sgRNA target sequence for a “double mutant nickase” is optionally provided to release torsion downstream of the 3′ homology arm close to the mutant ITR. Such embodiments increase annealing and promote HDR frequency.

In some embodiments, a nuclease can be inactivated/diminished after gene editing.

### F. Regulatory Switches

A molecular regulatory switch is one which generates a measurable change in state in response to a signal. Such regulatory switches can be usefully combined with the cleavable ceDNA vectors described herein to control the output of the cleavable ceDNA vector. In some embodiments, the cleavable ceDNA vector comprises a regulatory switch that serves to fine tune expression of the transgene.

In some embodiments, the cleavable ceDNA vector comprises more than one regulatory switch. In some embodiments, the regulatory switch is used to control the expression of a sequence in the transgene cassette. In some embodiments, the regulatory switch is used to control the expression of the site-specific nuclease enzyme and/or the gRNA.

In some embodiments, the regulatory switch can serve as a biocontainment function of the cleavable ceDNA vector. In some embodiments, the switch is an “ON/OFF” switch that is designed to start or stop (i.e., shut down) expression of the gene of interest in the cleavable ceDNA in a controllable and regulatable fashion. In some embodiments, the switch can include a “kill switch” that can instruct the cell comprising the cleavable ceDNA vector to undergo cell programmed death once the switch is activated. Exemplary regulatory switches encompassed by the disclosure are more fully discussed in PCT/US18/49996, which is incorporated herein in its entirety by reference

### (i) Binary Regulatory Switches

In some embodiments, the cleavable ceDNA vector comprises a regulatory switch that can serve to controllably modulate expression of the transgene. For example, the expression cassette of the cleavable ceDNA vector may additionally comprise a regulatory region, e.g., a promoter, cis-element, repressor, enhancer etc., that is operatively linked to the gene of interest, where the regulatory region is regulated by one or more cofactors or exogenous agents. By way of example only, regulatory regions can be modulated by small molecule switches or inducible or repressible promoters that regulate expression of, gene editing molecules, e.g., Cas nuclease. Nonlimiting examples of inducible promoters are hormone-inducible or metal-inducible promoters. Other exemplary inducible promoters/enhancer elements include, but are not limited to, an RU486-inducible promoter, an ecdysone-inducible promoter, a rapamycin-inducible promoter, and a metallothionein promoter.

(ii) Small molecule Regulatory Switches

A variety of art-known small-molecule based regulatory switches are known in the art and can be combined with the cleavable ceDNA vectors disclosed herein to form a regulatory-switch controlled ceDNA vector. In some embodiments, the regulatory switch can be selected from any one or a combination of: an orthogonal ligand/nuclear receptor pair, for example retinoid receptor variant/LG335 and GRQCIMFI, along with an artificial promoter controlling expression of the operatively linked transgene, such as that as disclosed in Taylor, et al. BMC Biotechnology 10 (2010): 15; engineered steroid receptors, e.g., modified progesterone receptor with a C-terminal truncation that cannot bind progesterone but binds RU486 (mifepristone) (U.S. Pat. No. 5,364,791); an ecdysone receptor from Drosophila and their ecdysteroid ligands (Saez, et al., PNAS, 97(26)(2000), 14512-14517; or a switch controlled by the antibiotic trimethoprim (TMP), as disclosed in Sando R 3^{rd}; Nat Methods. 2013, 10(11):1085-8. In some embodiments, the regulatory switch to control the transgene or expressed by the cleavable ceDNA vector is a pro-drug activation switch, such as that disclosed in U.S. Pat. Nos. 8,771,679, and 6,339,070.

(iii) “Passcode” Regulatory Switches

In some embodiments the regulatory switch can be a “passcode switch” or “passcode circuit”. Passcode switches allow fine tuning of the control of the expression of the transgene from the cleavable ceDNA vector when specific conditions occur—that is, a combination of conditions need to be present for transgene expression and/or repression to occur. For example, for expression of a transgene to occur at least conditions A and B must occur. A passcode regulatory switch can be any number of conditions, e.g., at least 2, or at least 3, or at least 4, or at least 5, or at least 6 or at least 7 or more conditions to be present for transgene expression to occur. In some embodiments, at least 2 conditions (e.g., A, B conditions) need to occur, and in some embodiments, at least 3 conditions need to occur (e.g., A, B and C, or A, B and D). By way of an example only, for gene expression from a cleavable ceDNA to occur that has a passcode “ABC” regulatory switch, conditions A, B and C must be present. Conditions A, B and C could be as follows; condition A is the presence of a condition or disease, condition B is a hormonal response, and condition C is a response to the transgene expression. For example, if the transgene edits a defective EPO gene, Condition A is the presence of Chronic Kidney Disease (CKD), Condition B occurs if the subject has hypoxic conditions in the kidney, Condition C is that Erythropoietin-producing cells (EPC) recruitment in the kidney is impaired; or alternatively, HIF-2 activation is impaired. Once the oxygen levels increase or the desired level of EPO is reached, the transgene turns off again until 3 conditions occur, turning it back on.

In some embodiments, a passcode regulatory switch or “Passcode circuit” encompassed for use in the cleavable ceDNA vector comprises hybrid transcription factors (TFs) to expand the range and complexity of environmental signals used to define biocontainment conditions. As opposed to a deadman switch which triggers cell death in the presence of a predetermined condition, the “passcode circuit” allows cell survival or transgene expression in the presence of a particular “passcode”, and can be easily reprogrammed to allow transgene expression and/or cell survival only when the predetermined environmental condition or passcode is present.

Any and all combinations of regulatory switches disclosed herein, e.g., small molecule switches, nucleic acid-based switches, small molecule-nucleic acid hybrid switches, post-transcriptional transgene regulation switches, post-translational regulation, radiation-controlled switches, hypoxia-mediated switches and other regulatory switches known by persons of ordinary skill in the art as disclosed herein can be used in a passcode regulatory switch as disclosed herein. Regulatory switches encompassed for use are also discussed in the review article Kis et al., J R Soc Interface. 12: 20141000 (2015), and summarized in Table 1 of Kis. In some embodiments, a regulatory switch for use in a passcode system can be selected from any or a combination of the switches in Table 11.

### (iv). Nucleic Acid-Based Regulatory Switches to Control Transgene Expression

In some embodiments, the regulatory switch to control the transgene expressed by the cleavable ceDNA is based on a nucleic-acid based control mechanism. Exemplary nucleic acid control mechanisms are known in the art and are envisioned for use. For example, such mechanisms include riboswitches, such as those disclosed in, e.g., 1JS2009/0305253, US2008/0269258, US2017/0204477, WO2018026762A1, U.S. Pat. No. 9,222,093 and EP application EP288071, and also disclosed in the review by Villa J K et al., Microbiol Spectr. 2018 May; 6(3). Also included are metabolite-responsive transcription biosensors, such as those disclosed in WO2018/075486 and WO2017/147585. Other art-known mechanisms envisioned for use include silencing of the transgene with an siRNA or RNAi molecule (e.g., miR, shRNA). For example, the cleavable ceDNA vector can comprise a regulatory switch that encodes a RNAi molecule that is complementary to the transgene expressed by the cleavable ceDNA vector. When such RNAi is expressed even if the transgene is expressed by the cleavable ceDNA vector, it will be silenced by the complementary RNAi molecule, and when the RNAi is not expressed when the transgene is expressed by the cleavable ceDNA vector the transgene is not silenced by the RNAi.

In some embodiments, the regulatory switch is a tissue-specific self-inactivating regulatory switch, for example as disclosed in US2002/0022018, whereby the regulatory switch deliberately switches transgene expression off at a site where transgene expression might otherwise be disadvantageous. In some embodiments, the regulatory switch is a recombinase reversible gene expression system, for example as disclosed in US2014/0127162 and U.S. Pat. No. 8,324,436.

### (v). Post-Transcriptional and Post-Translational Regulatory Switches.

In some embodiments, the regulatory switch to control the transgene or gene of interest expressed by the cleavable ceDNA vector is a post-transcriptional modification system. For example, such a regulatory switch can be an aptazyme riboswitch that is sensitive to tetracycline or theophylline, as disclosed in US2018/0119156, GB201107768, WO2001/064956A3, EP Patent 2707487 and Beilstein et al., ACS Synth. Biol., 2015, 4 (5), pp 526-534; Zhong et al., Elife. 2016 Nov. 2; 5. pii: e18858. In some embodiments, it is envisioned that a person of ordinary skill in the art could encode both the transgene and an inhibitory siRNA which contains a ligand sensitive (OFF-switch) aptamer, the net result being a ligand sensitive ON-switch.

### (vi). Other Exemplary Regulatory Switches

Any known regulatory switch can be used in the cleavable ceDNA vector to control the gene expression of the transgene expressed by the cleavable ceDNA vector, including those triggered by environmental changes. Additional examples include, but are not limited to; the BOC method of Suzuki et al., Scientific Reports 8; 10051 (2018); genetic code expansion and a non-physiologic amino acid; radiation-controlled or ultra-sound controlled on/off switches (see, e.g., Scott S et al., Gene Ther. 2000 July; 7(13):1121-5; U.S. Pat. Nos. 5,612,318; 5,571,797; 5,770,581; 5,817,636; and WO1999/025385A1. In some embodiments, the regulatory switch is controlled by an implantable system, e.g., as disclosed in U.S. Pat. No. 7,840,263; US2007/0190028A 1 where gene expression is controlled by one or more forms of energy, including electromagnetic energy, that activates promoters operatively linked to the transgene in the cleavable ceDNA vector.

In some embodiments, a regulatory switch envisioned for use in the cleavable ceDNA vector is a hypoxia-mediated or stress-activated switch, e.g., such as those disclosed in WO1999060142A2, U.S. Pat. No. 5,834,306:6,218,179; 6,709,858; US2015/0322410; Greco et al., (2004) Targeted Cancer Therapies 9, S368, as well as FROG, TOAD and NRSE elements and conditionally inducible silence elements, including hypoxia response elements (HREs), inflammatory response elements (IREs) and shear-stress activated elements (SSAEs), e.g., as disclosed in U.S. Pat. No. 9,394,526. Such an embodiment is useful for turning on expression of the transgene from the cleavable ceDNA vector after ischemia or in ischemic tissues, and/or tumors.

### (iv). Kill Switches

Other embodiments of the invention relate to a cleavable ceDNA vector comprising a kill switch. A kill switch as disclosed herein enables a cell comprising the cleavable ceDNA vector to be killed or undergo programmed cell death as a means to permanently remove an introduced ceDNA vector from the subject's system. It will be appreciated by one of ordinary skill in the art that use of kill switches in the cleavable ceDNA vectors of the invention would be typically coupled with targeting of the cleavable ceDNA vector to a limited number of cells that the subject can acceptably lose or to a cell type where apoptosis is desirable (e.g., cancer cells). In all aspects, a “kill switch” as disclosed herein is designed to provide rapid and robust cell killing of the cell comprising the cleavable ceDNA vector in the absence of an input survival signal or other specified condition. Stated another way, a kill switch encoded by a cleavable ceDNA vector herein can restrict cell survival of a cell comprising a cleavable ceDNA vector to an environment defined by specific input signals. Such kill switches serve as a biological biocontainment function should it be desirable to remove the cleavable ceDNA vector from a subject or to ensure that it will not express the encoded transgene.

### VII. Detailed Method of Production of a ceDNA Vector

### A. Production in General

Certain methods for the production of a cleavable ceDNA comprising an asymmetrical ITR pair or symmetrical ITR pair as defined herein is described in section IV of PCT/US18/49996 filed Sep. 7, 2018, which is incorporated herein in its entirety by reference. As described herein, the cleavable ceDNA vector can be obtained, for example, by the process comprising the steps of: a) incubating a population of host cells (e.g., insect cells) harboring the polynucleotide expression construct template (e.g., a cleavable ceDNA-plasmid, a cleavable ceDNA-Bacmid, and/or a cleavable ceDNA-baculovirus), which is devoid of viral capsid coding sequences, in the presence of a Rep protein under conditions effective and for a time sufficient to induce production of the cleavable ceDNA vector within the host cells, and wherein the host cells do not comprise viral capsid coding sequences; and b) harvesting and isolating the cleavable ceDNA vector from the host cells. The presence of Rep protein induces replication of the vector polynucleotide with a modified ITR to produce the cleavable ceDNA vector in a host cell. However, no viral particles (e.g., AAV virions) are expressed. Thus, there is no size limitation such as that naturally imposed in AAV or other viral-based vectors.

The presence of the cleavable ceDNA vector isolated from the host cells can be confirmed by digesting DNA isolated from the host cell with a restriction enzyme having a single recognition site on the cleavable ceDNA vector and analyzing the digested DNA material on a non-denaturing gel to confirm the presence of characteristic bands of linear and continuous DNA as compared to linear and non-continuous DNA.

In yet another aspect, the invention provides for use of host cell lines that have stably integrated the DNA vector polynucleotide expression template (ceDNA template) into their own genome in production of the non-viral DNA vector, e.g., as described in Lee, L. et al. (2013) Plos One 8(8): e69879. Preferably, Rep is added to host cells at an MOI of about 3. When the host cell line is a mammalian cell line, e.g., HEK293 cells, the cell lines can have polynucleotide vector template stably integrated, and a second vector such as herpes virus can be used to introduce Rep protein into cells, allowing for the excision and amplification of ceDNA in the presence of Rep and helper virus.

In one embodiment, the host cells used to make the cleavable ceDNA vectors described herein are insect cells, and baculovirus is used to deliver both the polynucleotide that encodes Rep protein and the non-viral DNA vector polynucleotide expression construct template for ceDNA, e.g., as described in Example 1. In some embodiments, the host cell is engineered to express Rep protein.

The cleavable ceDNA vector is then harvested and isolated from the host cells. The time for harvesting and collecting ceDNA vectors described herein from the cells can be selected and optimized to achieve a high-yield production of the cleavable ceDNA vectors. For example, the harvest time can be selected in view of cell viability, cell morphology, cell growth, etc. In one embodiment, cells are grown under sufficient conditions and harvested a sufficient time after baculoviral infection to produce ceDNA vectors but before a majority of cells start to die because of the baculoviral toxicity. The DNA vectors can be isolated using plasmid purification kits such as Qiagen Endo-Free Plasmid kits. Other methods developed for plasmid isolation can be also adapted for DNA vectors. Generally, any nucleic acid purification methods can be adopted.

The DNA vectors can be purified by any means known to those of skill in the art for purification of DNA. In one embodiment, ceDNA vectors are purified as DNA molecules. In another embodiment, the cleavable ceDNA vectors are purified as exosomes or microparticles.

The presence of the cleavable ceDNA vector can be confirmed by digesting the vector DNA isolated from the cells with a restriction enzyme having a single recognition site on the DNA vector and analyzing both digested and undigested DNA material using gel electrophoresis to confirm the presence of characteristic bands of linear and continuous DNA as compared to linear and non-continuous DNA.

Alternatively, cleavable ceDNA vectors of the present invention are produced synthetically in a cell-free environment utilizing the procedures described in International Patent Application Publication No. WO20191//143885.

B. ceDNA Plasmid

A cleavable ceDNA-plasmid is a plasmid used for later production of a cleavable ceDNA vector. In some embodiments, a cleavable ceDNA-plasmid can be constructed using known techniques to provide at least the following as operatively linked components in the direction of transcription: (1) a modified 5′ ITR sequence; (2) an expression cassette containing a cis-regulatory element, for example, a promoter, inducible promoter, regulatory switch, enhancers and the like; and (3) a modified 3′ ITR sequence, where the 3′ ITR sequence is symmetric relative to the 5′ ITR sequence. In some embodiments, the expression cassette flanked by the ITRs comprises a cloning site for introducing an exogenous sequence. The expression cassette replaces the rep and cap coding regions of the AAV genomes.

In one aspect, a cleavable ceDNA vector is obtained from a plasmid, referred to herein as a “ceDNA-plasmid” encoding in this order: a first adeno-associated virus (AAV) inverted terminal repeat (ITR), an expression cassette comprising a transgene, and a mutated or modified AAV ITR, wherein said ceDNA-plasmid is devoid of AAV capsid protein coding sequences. In alternative embodiments, the cleavable ceDNA-plasmid encodes in this order: a first (or 5′) modified or mutated AAV ITR, an expression cassette comprising a transgene, and a second (or 3′) modified AAV ITR, wherein said ceDNA-plasmid is devoid of AAV capsid protein coding sequences, and wherein the 5′ and 3′ ITRs are symmetric relative to each other. In alternative embodiments, the cleavable ceDNA-plasmid encodes in this order: a first (or 5′) modified or mutated AAV ITR, an expression cassette comprising a transgene, and a second (or 3′) mutated or modified AAV ITR, wherein said ceDNA-plasmid is devoid of AAV capsid protein coding sequences, and wherein the 5′ and 3′ modified ITRs are have the same modifications (i.e., they are inverse complement or symmetric relative to each other).

In a further embodiment, the cleavable ceDNA-plasmid system is devoid of viral capsid protein coding sequences (i.e. it is devoid of AAV capsid genes but also of capsid genes of other viruses). In addition, in a particular embodiment, the cleavable ceDNA-plasmid is also devoid of AAV Rep protein coding sequences. Accordingly, in a preferred embodiment, ceDNA-plasmid is devoid of functional AAV cap and AAV rep genes GG-3′ for AAV2) plus a variable palindromic sequence allowing for hairpin formation.

A cleavable ceDNA-plasmid of the present invention can be generated using natural nucleotide sequences of the genomes of any AAV serotypes well known in the art. In one embodiment, the cleavable ceDNA-plasmid backbone is derived from the AAV1, AAV2, AAV3, AAV4, AAV5, AAV 5, AAV7, AAV8, AAV9, AAV10, AAV 11, AAV12, AAVrh8, AAVrh10, AAV-DJ, and AAV-DJ8 genome. E.g., NCBI: NC 002077; NC 001401; NC001729: NC001829; NC006152; NC 006260: NC 006261: Kotin and Smith, The Springer Index of Viruses, available at the URL maintained by Springer (at www web address: oesys.springer.de/viruses/database/mkchapter.asp?virID=42.04.(note—references to a URL or database refer to the contents of the URL or database as of the effective filing date of this application) In a particular embodiment, the cleavable ceDNA-plasmid backbone is derived from the AAV2 genome. In another particular embodiment, the cleavable ceDNA-plasmid backbone is a synthetic backbone genetically engineered to include at its 5′ and 3′ ITRs derived from one of these AAV genomes.

A cleavable ceDNA-plasmid can optionally include a selectable or selection marker for use in the establishment of a cleavable ceDNA vector-producing cell line. In one embodiment, the selection marker can be inserted downstream (i.e., 3′) of the 3′ ITR sequence. In another embodiment, the selection marker can be inserted upstream (i.e., 5′) of the 5′ ITR sequence. Appropriate selection markers include, for example, those that confer drug resistance. Selection markers can be, for example, a blasticidin S-resistance gene, kanamycin, geneticin, and the like. In a preferred embodiment, the drug selection marker is a blasticidin S-resistance gene.

An Exemplary ceDNA (e.g., rAAV0) is produced from an rAAV plasmid. A method for the production of a rAAV vector, can comprise: (a) providing a host cell with a rAAV plasmid as described above, wherein both the host cell and the plasmid are devoid of capsid protein encoding genes, (b) culturing the host cell under conditions allowing production of an ceDNA genome, and (c) harvesting the cells and isolating the AAV genome produced from said cells.

C. Exemplary Method of Making the ceDNA Vectors from ceDNA Plasmids

Methods for making capsid-less ceDNA vectors are also provided herein, notably a method with a sufficiently high yield to provide sufficient vector for in vivo experiments.

In some embodiments, a method for the production of a cleavable ceDNA vector comprises the steps of: (1) introducing the nucleic acid construct comprising an expression cassette and two symmetric ITR sequences into a host cell (e.g., Sf9 cells), (2) optionally, establishing a clonal cell line, for example, by using a selection marker present on the plasmid, (3) introducing a Rep coding gene (either by transfection or infection with a baculovirus carrying said gene) into said insect cell, and (4) harvesting the cell and purifying the cleavable ceDNA vector. The nucleic acid construct comprising an expression cassette and two ITR sequences described above for the production of ceDNA vector can be in the form of a cleavable ceDNA plasmid, or Bacmid or Baculovirus generated with the cleavable ceDNA plasmid as described below. The nucleic acid construct can be introduced into a host cell by transfection, viral transduction, stable integration, or other methods known in the art.

### D. Cel Lines

Host cell lines used in the production of a cleavable ceDNA vector can include insect cell lines derived from Spodoptera frugiperda, such as Sf9 Sf21, or Trichoplusia ni cell, or other invertebrate, vertebrate, or other eukaryotic cell lines including mammalian cells. Other cell lines known to an ordinarily skilled artisan can also be used, such as HEK293, Huh-7, HeLa, HepG2, HeplA, 911, CHO, COS, MeWo, NIH3T3, A549, HT1 180, monocytes, and mature and immature dendritic cells. Host cell lines can be transfected for stable expression of the cleavable ceDNA-plasmid for high yield ceDNA vector production.

ceDNA-plasmids can be introduced into Sf9 cells by transient transfection using reagents (e.g., liposomal, calcium phosphate) or physical means (e.g., electroporation) known in the art. Alternatively, stable Sf9 cell lines which have stably integrated the cleavable ceDNA-plasmid into their genomes can be established. Such stable cell lines can be established by incorporating a selection marker into the cleavable ceDNA—plasmid as described above. If the cleavable ceDNA—plasmid used to transfect the cell line includes a selection marker, such as an antibiotic, cells that have been transfected with the cleavable ceDNA-plasmid and integrated the cleavable ceDNA-plasmid DNA into their genome can be selected for by addition of the antibiotic to the cell growth media. Resistant clones of the cells can then be isolated by single-cell dilution or colony transfer techniques and propagated.

Alternatively, as mentioned above, cleavable ceDNA vectors of the present invention are produced synthetically in a cell-free environment utilizing the procedures described in International Patent Application Publication No. WO2019//143885, content of which is incorporated herein by reference in its entirety.

E. Isolating and Purifying ceDNA Vectors

Cleavable ceDNA-vectors disclosed herein can be obtained from a producer cell expressing AAV Rep protein(s), further transformed with a cleavable ceDNA-plasmid, ceDNA-bacmid, or ceDNA-baculovirus.

In one aspect, a polynucleotide encodes the AAV Rep protein (Rep 78 or 68) delivered to a producer cell in a plasmid (Rep-plasmid), a bacmid (Rep-bacmid), or a baculovirus (Rep-baculovirus). The Rep-plasmid, Rep-bacmid, and Rep-baculovirus can be generated by methods described above.

Methods to produce a cleavable ceDNA-vector, which is an exemplary ceDNA vector, are described herein. Expression constructs used for generating a cleavable ceDNA vectors of the present invention can be a plasmid (e.g., ceDNA-plasmids), a Bacmid (e.g., ceDNA-bacmid), and/or a baculovirus (e.g., ceDNA-baculovirus). By way of an example only, a cleavable ceDNA-vector can be generated from the cells co-infected with ceDNA-baculovirus and Rep-baculovirus. Rep proteins produced from the Rep-baculovirus can replicate the cleavable ceDNA-baculovirus to generate ceDNA-vectors. Alternatively, ceDNA vectors can be generated from the cells stably transfected with a construct comprising a sequence encoding the AAV Rep protein (Rep78/52) delivered in Rep-plasmids, Rep-bacmids, or Rep-baculovirus. CeDNA-Baculovirus can be transiently transfected to the cells, be replicated by Rep protein and produce ceDNA vectors.

The bacmid (e.g., ceDNA-bacmid) can be transfected into a permissive insect cells such as Sf9, Sf21, Tni (Trichoplusia ni) cell, High Five cell, and generate ceDNA-baculovirus, which is a recombinant baculovirus including the sequences comprising the symmetric ITRs and the expression cassette. ceDNA-baculovirus can be again infected into the insect cells to obtain a next generation of the recombinant baculovirus. Optionally, the step can be repeated once or multiple times to produce the recombinant baculovirus in a larger quantity.

The time for harvesting and collecting ceDNA vectors described herein from the cells can be selected and optimized to achieve a high-yield production of the cleavable ceDNA vectors. For example, the harvest time can be selected in view of cell viability, cell morphology, cell growth, etc. Usually, cells can be harvested after sufficient time after baculoviral infection to produce ceDNA vectors (e.g., ceDNA vectors) but before majority of cells start to die because of the viral toxicity. The cleavable ceDNA-vectors can be isolated from the Sf9 cells using plasmid purification kits such as Qiagen ENDO-FREE PLASMID® kits. Other methods developed for plasmid isolation can be also adapted for ceDNA vectors. Generally, any art-known nucleic acid purification methods can be adopted, as well as commercially available DNA extraction kits.

Alternatively, purification can be implemented by subjecting a cell pellet to an alkaline lysis process, centrifuging the resulting lysate and performing chromatographic separation. As one nonlimiting example, the process can be performed by loading the supernatant on an ion exchange column (e.g., SARTOBIND Q®) which retains nucleic acids, and then eluting (e.g., with a 1.2 M NaCl solution) and performing a further chromatographic purification on a gel filtration column (e.g., 6 fast flow GE). The capsid-free AAV vector is then recovered by, e.g., precipitation.

In some embodiments, ceDNA vectors can also be purified in the form of exosomes, or microparticles. It is known in the art that many cell types release not only soluble proteins, but also complex protein/nucleic acid cargoes via membrane microvesicle shedding (Cocucci et al, 2009; EP 10306226.1) Such vesicles include microvesicles (also referred to as microparticles) and exosomes (also referred to as nanovesicles), both of which comprise proteins and RNA as cargo. Microvesicles are generated from the direct budding of the plasma membrane, and exosomes are released into the extracellular environment upon fusion of multivesicular endosomes with the plasma membrane. Thus, ceDNA vector-containing microvesicles and/or exosomes can be isolated from cells that have been transduced with the cleavable ceDNA-plasmid or a bacmid or baculovirus generated with the cleavable ceDNA-plasmid.

Microvesicles can be isolated by subjecting culture medium to filtration or ultracentrifugation at 20,000×g, and exosomes at 100,000×g. The optimal duration of ultracentrifugation can be experimentally-determined and will depend on the particular cell type from which the vesicles are isolated. Preferably, the culture medium is first cleared by low-speed centrifugation (e.g., at 2000×g for 5-20 minutes) and subjected to spin concentration using, e.g., an AMICON® spin column (Millipore, Watford, UK). Microvesicles and exosomes can be further purified via FACS or MACS by using specific antibodies that recognize specific surface antigens present on the microvesicles and exosomes. Other microvesicle and exosome purification methods include, but are not limited to, immunoprecipitation, affinity chromatography, filtration, and magnetic beads coated with specific antibodies or aptamers. Upon purification, vesicles are washed with, e.g., phosphate-buffered saline. One advantage of using microvesicles or exosome to deliver ceDNA-containing vesicles is that these vesicles can be targeted to various cell types by including on their membranes proteins recognized by specific receptors on the respective cell types. (See also EP 10306226)

Another aspect of the invention herein relates to methods of purifying ceDNA vectors from host cell lines that have stably integrated a cleavable ceDNA construct into their own genome. In one embodiment, ceDNA vectors are purified as DNA molecules. In another embodiment, the cleavable ceDNA vectors are purified as exosomes or microparticles.

FIG. 5 of PCT/US18/49996 shows a gel confirming the production of ceDNA from multiple ceDNA-plasmid constructs using the method described in the Examples. The cleavable ceDNA is confirmed by a characteristic band pattern in the gel

### VIII. Pharmaceutical Compositions

In another aspect, pharmaceutical compositions are provided. The pharmaceutical composition comprises a cleavable ceDNA as disclosed herein and a pharmaceutically acceptable carrier or diluent. The pharmaceutical composition may further comprise a nuclease molecule (e.g., a Cas enzyme) and/or one or more guide RNAs.

The closed-ended DNA disclosed herein can be incorporated into pharmaceutical compositions suitable for administration to a subject for in vivo delivery to cells, tissues, or organs of the subject, alone or in combination with a nuclease molecule and/or one or more gRNAs. Typically, the pharmaceutical composition comprises a cleavable ceDNA-vector as disclosed herein and a pharmaceutically acceptable carrier. For example, the cleavable ceDNA vectors described herein can be incorporated into a pharmaceutical composition suitable for a desired route of therapeutic administration (e.g., parenteral administration). Passive tissue transduction via high pressure intravenous or intra-arterial infusion, as well as intracellular injection, such as intranuclear microinjection or intracytoplasmic injection, are also contemplated. Pharmaceutical compositions for therapeutic purposes can be formulated as a solution, microemulsion, dispersion, liposomes, or other ordered structure suitable to high ceDNA vector concentration. Sterile injectable solutions can be prepared by incorporating the cleavable ceDNA vector compound in the required amount in an appropriate buffer with one or a combination of ingredients enumerated above, as required, followed by filtered sterilization including a cleavable ceDNA vector can be formulated to deliver a transgene in the nucleic acid to the cells of a recipient, resulting in the therapeutic expression of the transgene or donor sequence therein. The composition can also include a pharmaceutically acceptable carrier.

Pharmaceutically active compositions comprising a cleavable ceDNA vector can be formulated to deliver a transgene or donor sequence for various purposes to the cell, e.g., cells of a subject.

Pharmaceutical compositions for therapeutic purposes typically must be sterile and stable under the conditions of manufacture and storage. The composition can be formulated as a solution, microemulsion, dispersion, liposomes, or other ordered structure suitable to high ceDNA vector concentration. Sterile injectable solutions can be prepared by incorporating the cleavable ceDNA vector compound in the required amount in an appropriate buffer with one or a combination of ingredients enumerated above, as required, followed by filtered sterilization.

A cleavable ceDNA vector as disclosed herein can be incorporated into a pharmaceutical composition suitable for topical, systemic, intra-amniotic, intrathecal, intracranial, intra-arterial, intravenous, intralymphatic, intraperitoneal, subcutaneous, tracheal, intra-tissue (e.g., intramuscular, intracardiac, intrahepatic, intrarenal, intracerebral), intrathecal, intravesical, conjunctival (e.g., extra-orbital, intraorbital, retroorbital, intraretinal, subretinal, choroidal, sub-choroidal, intrastromal, intracameral and intravitreal), intracochlear, and mucosal (e.g., oral, rectal, nasal) administration. Passive tissue transduction via high pressure intravenous or intraarterial infusion, as well as intracellular injection, such as intranuclear microinjection or intracytoplasmic injection, are also contemplated.

Pharmaceutical compositions for therapeutic purposes typically must be sterile and stable under the conditions of manufacture and storage. The composition can be formulated as a solution, microemulsion, dispersion, liposomes, or other ordered structure suitable to high ceDNA vector concentration. Sterile injectable solutions can be prepared by incorporating the cleavable ceDNA vector compound in the required amount in an appropriate buffer with one or a combination of ingredients enumerated above, as required, followed by filtered sterilization.

In some aspects, the methods provided herein comprise delivering one or more cleaved ceDNA vectors in combination with a nuclease enzyme, e.g., a Cas enzyme, and a gRNA, for gene editing as disclosed herein to a host cell. Also provided herein are cells produced by such methods, and organisms (such as animals, plants, or fungi) comprising or produced from such cells. Methods of delivery of nucleic acids can include lipofection, nucleofection, microinjection, biolistics, liposomes, immunoliposomes, polycation or lipid:nucleic acid conjugates, naked DNA, and agent-enhanced uptake of DNA. Lipofection is described in e.g., U.S. Pat. Nos. 5,049,386, 4,946,787; and 4,897,355) and lipofection reagents are sold commercially (e.g., TRANSFECTAM™ and LPOFECTIN™). Delivery can be to cells (e.g., in vitro or ex vivo administration) or target tissues (e.g., in vivo administration).

Various techniques and methods are known in the art for delivering nucleic acids to cells. For example, nucleic acids, such as ceDNA can be formulated into lipid nanoparticles (LNPs), lipidoids, liposomes, lipid nanoparticles, lipoplexes, or core-shell nanoparticles. Typically, LNPs are composed of nucleic acid (e.g., ceDNA) molecules, one or more ionizable or cationic lipids (or salts thereof), one or more non-ionic or neutral lipids (e.g., a phospholipid), a molecule that prevents aggregation (e.g., PEG or a PEG-lipid conjugate), and optionally a sterol (e.g., cholesterol).

Another method for delivering nucleic acids, such as ceDNA to a cell is by conjugating the nucleic acid with a ligand that is internalized by the cell. For example, the ligand can bind a receptor on the cell surface and internalized via endocytosis. The ligand can be covalently linked to a nucleotide in the nucleic acid. Exemplary conjugates for delivering nucleic acids into a cell are described, example, in WO2015/006740, WO2014/025805, WO2012/037254, WO2009/082606, WO2009/073809, WO2009/018332, WO2006/112872, WO2004/090108, WO2004/091515 and WO2017/177326.

Nucleic acids, such as ceDNA, can also be delivered to a cell by transfection. Useful transfection methods include, but are not limited to, lipid-mediated transfection, cationic polymer-mediated transfection, or calcium phosphate precipitation. Transfection reagents are well known in the art and include, but are not limited to, TurboFect Transfection Reagent (Thermo Fisher Scientific), Pro-Ject Reagent (Thermo Fisher Scientific), TRANSPASS™ P Protein Transfection Reagent (New England Biolabs), CHARIOT™ Protein Delivery Reagent (Active Motif), PROTEOJUICE™ Protein Transfection Reagent (EMD Millipore), 293fectin, LIPOFECTAMINE™ 2000, LIPOFECTAMINE™ 3000 (Thermo Fisher Scientific), LIPOFECTAMINE™ (Thermo Fisher Scientific), LIPOFECTIN™ (Thermo Fisher Scientific), DMRIE-C, CELLFECTIN™ (Thermo Fisher Scientific), OLIGOFECTAMINE™ (Thermo Fisher Scientific), LIPOFECTACE™, FUGENE™ (Roche, Basel, Switzerland), FUGENE™ HD (Roche), TRANSFECTAM™ (Transfectam, Promega, Madison, Wis.), TFX-10™ (Promega), TFX-20™ (Promega), TFX-50™ (Promega), TRANSFECTIN™ (BioRad, Hercules, Calif.), SILENTFECT™ (Bio-Rad), Effectene™ (Qiagen, Valencia, Calif.), DC-chol (Avanti Polar Lipids), GENEPORTER™ (Gene Therapy Systems, San Diego, Calif.), DHARMAFECT 1™ (Dharmacon, Lafayette, Colo.), DHARMAFECT 2™ (Dharmacon), DHARMAFECT 3™ (Dharmacon), DHARMAFECT 4rm (Dharmacon), ESCORT™ III (Sigma, St. Louis. Mo.), and ESCORT™ IV (Sigma Chemical Co.). Nucleic acids, such as ceDNA, can also be delivered to a cell via microfluidics methods known to those of skill in the art.

Methods of non-viral delivery of nucleic acids in vivo or ex vivo include electroporation, lipofection (see, U.S. Pat. No. 5,049,386; 4,946,787 and commercially available reagents such as Transfectam™ and Lipofectin™), microinjection, biolistics, virosomes, liposomes (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787), immunoliposomes, polycation or lipid:nucleic acid conjugates, naked DNA, and agent-enhanced uptake of DNA. Sonoporation using, e.g., the Sonitron 2000 system (Rich-Mar) can also be used for delivery of nucleic acids.

ceDNA vectors as described herein can also be administered directly to an organism for transduction of cells in vivo. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Methods for introduction of a nucleic acid vector ceDNA vector as disclosed herein can be delivered into hematopoietic stem cells, for example, by the methods as described, for example, in U.S. Pat. No. 5,928,638.

Delivery reagents such as liposomes, nanocapsules, microparticles, microspheres, lipid particles, vesicles, and the like, can be used for the introduction of the compositions of the present disclosure into suitable host cells. In particular, the nucleic acids can be formulated for delivery either encapsulated in a lipid particle, a liposome, a vesicle, a nanosphere, a nanoparticle, a gold particle, or the like. Such formulations can be preferred for the introduction of pharmaceutically acceptable formulations of the nucleic acids disclosed herein.

In some embodiments, a cleavable ceDNA vector alone is directly injected as naked DNA into skin, thymus, cardiac muscle, skeletal muscle, or liver cells.

In some cases, a cleavable ceDNA vector is delivered by hydrodynamic injection, which is a simple and highly efficient method for direct intracellular delivery of any water-soluble compounds and particles into internal organs and skeletal muscle in an entire limb.

### A. Exosomes

In some embodiments, a cleavable ceDNA vector as disclosed herein is delivered by being packaged in an exosome. Exosomes are small membrane vesicles of endocytic origin that are released into the extracellular environment following fusion of multivesicular bodies with the plasma membrane. Their surface consists of a lipid bilayer from the donor cell's cell membrane, they contain cytosol from the cell that produced the exosome, and exhibit membrane proteins from the parental cell on the surface. Exosomes are produced by various cell types including epithelial cells, B and T lymphocytes, mast cells (MC) as well as dendritic cells (DC). Some embodiments, exosomes with a diameter between 10 nm and 1 μm, between 20 nm and 500 nm, between 30 nm and 250 nm, between 50 nm and 100 nm are envisioned for use. Exosomes can be isolated for a delivery to target cells using either their donor cells or by introducing specific nucleic acids into them. Various approaches known in the art can be used to produce exosomes containing capsid-free AAV vectors of the present invention.

### B. Lipid Particles or Lipid Nanoparticles

In some embodiments, a cleavable ceDNA vector as disclosed herein is delivered by a lipid particle or nanoparticle. Generally, lipid nanoparticles comprise an ionizable amino lipid (e.g., heptatriaconta-6,9,28,31-tetraen-19-yl 4-(dimethylamino)butanoate, DLin-MC3-DMA), and least one other lipid class selected from a sterol, a non-cationic lipid, and a PEGylated lipid. Lipid nanoparticles (LNPs), or pharmaceutical compositions thereof, comprising an ionizable or cationic lipid described herein and a capsid free, non-viral vector (e.g., cleavable ceDNA) can be used to deliver the capsid-free, non-viral cleavable DNA vector to a target site of interest (e.g., cell, tissue, organ, and the like). Accordingly, another aspect of this disclosure relates to a lipid nanoparticle (LNP) comprising one or more ionizable lipids described herein, or a pharmaceutically acceptable salt thereof and cleavable ceDNA. In one embodiment, the gene editing systems as described herein comprises the cleavable ceDNA formulated as a lipid nanoparticle composition. In one embodiment, the gene editing systems as described herein further comprises at least one gRNA and at least one site-specific nuclease enzyme or a messenger ribonucleic acid (mRNA) encoding the at least one site-specific nuclease enzyme formulated as a lipid nanoparticle composition. In one embodiment, the cleavable ceDNA is formulated as a first lipid nanoparticle composition and the at least one gRNA, and the at least one site-specific nuclease enzyme or a messenger ribonucleic acid (mRNA) encoding the at least one site-specific nuclease enzyme are formulated as a second lipid nanoparticle composition.

Generally, an ionizable or cationic lipid is typically employed to condense the nucleic acid cargo, e.g., ceDNA at low pH and to drive membrane association and fusogenicity. Generally, cationic lipids are lipids comprising at least one amino group that is positively charged or becomes protonated under acidic conditions, for example at pH of 6.5 or lower, to form lipids comprising quaternary amines.

In one embodiment of any of the aspects or embodiments herein, in a lipid nanoparticle, the cationic lipid as provided herein or a pharmaceutically acceptable salt thereof is present at a molar percentage of about 30% to about 80%, e.g., about 35% to about 80%, about 40% to about 80%, about 45% to about 80%, about 50% to about 80%, about 55% to about 80%, about 60% to about 80%, about 65% to about 80%, about 70% to about 80%, about 75% to about 80%, 30% to about 75%, about 35% to about 75%, about 40% to about 75%, about 45% to about 75%, about 50% to about 75%, about 55% to about 75%, about 60% to about 75%, about 65% to about 75%, about 70% to about 75%, 30% to about 70%, about 35% to about 70%, about 40% to about 70%, about 45% to about 70%, about 50% to about 70%, about 55% to about 70%, about 60% to about 70%, about 65% to about 70%, about 30% to about 65%, about 35% to about 65%, about 40% to about 65%, about 45% to about 65%, about 50% to about 65%, about 55% to about 65%, about 60% to about 65%, about 30% to about 60%, about 35% to about 60%, about 40% to about 60%, about 45% to about 60%, about 50% to about 60%, about 55% to about 60%, about 30% to about 55%, about 35% to about 55%, about 40% to about 55%, about 45% to about 55%, about 50% to about 55%, about 30% to about 50%, about 35% to about 50%, about 40% to about 50%, about 45% to about 50%, about 30% to about 45%, about 35% to about 45%, about 40% to about 45%, about 30% to about 40%, or about 35% to about 40%. In one embodiment of any of the aspects or embodiments herein, in a lipid nanoparticle, the cationic lipid as provided herein or a pharmaceutically acceptable salt thereof is present at a molar percentage of about 40% to about 60%, or about 45% to about 60%, or about 45% to about 55%, or about 45% to about 50%, or about 50% to about 55%, or about 40% to about 50%; such as but not limited to about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, or about 60%.

### Sterol

In one embodiment of any of the aspects or embodiments herein, in addition to the more cationic lipids described herein, or a pharmaceutically acceptable salt thereof, and a cleavable ceDNA, the LNP described herein further comprises at least one sterol, to provide membrane integrity and stability of the lipid particle. In one embodiment of any of the aspects or embodiments herein, an exemplary sterol that can be used in the lipid particle is cholesterol, or a derivative thereof. Non-limiting examples of cholesterol derivatives include polar analogues such as 5α-cholestanol, 5β-coprostanol, cholesteryl-(2′-hydroxy)-ethyl ether, cholesteryl-(4′-hydroxy)-butyl ether, and 6-ketocholestanol; non-polar analogues such as 5α-cholestane, cholestenone, 5α-cholestanone, 5β-cholestanone, and cholesteryl decanoate; and mixtures thereof. In some embodiments of any of the aspects and embodiments herein, the cholesterol derivative is a polar analogue such as cholesteryl-(4′-hydroxy)-butyl ether. In some embodiments of any of the aspects and embodiments herein, cholesterol derivative is cholestryl hemisuccinate (CHEMS).

Exemplary cholesterol derivatives are described in International Patent Application Publication No. WO2009/127060 and U.S. Patent Application Publication No. US2010/0130588, contents of both of which are incorporated herein by reference in their entirety.

Further exemplary sterols include betasitosterol, campesterol, stigmasterol, ergosterol, brassicasterol, lopeol, cycloartenol, and derivatives thereof. In one embodiment of any of the aspects or embodiments herein, an exemplary sterol that can be used in the lipid particle is betasitosterol.

In one embodiment of any of the aspects or embodiments herein, in a lipid nanoparticle, the sterol is present at a molar percentage of about 20% to about 50%, e.g., about 25% to about 50%, about 30% to about 50%, about 35% to about 50%, about 40% to about 50%, about 45% to about 50%, about 20% to about 45%, about 25% to about 45%, about 30% to about 45%, about 35% to about 45%, about 40% to about 45%, about 20% to about 40%, about 25% to about 40%, about 30% to about 40%, about 35% to about 40%, about 20% to about 35%, about 25% to about 35%, about 30% to about 35%, about 20% to about 30%, or about 25% to about 35%. In one embodiment of any of the aspects or embodiments herein, in a lipid nanoparticle, the sterol is present at a molar percentage of about 35% to about 45%, or about 40% to about 45%, or about 35% to about 40%; such as but not limited to about 35%, about 36%, about 37%, about 38%, about 39%, about 40%, about 41%, about 42%, about 43%, about 44%, or about 45%.

### Non-Cationic Lipids

In one embodiment of any of the aspects or embodiments herein, a lipid nanoparticle (LNP) described herein further comprises at least one non-cationic lipid. Non-cationic lipids are also known as structural lipids, and may serve to increase fusogenicity and also increase stability of the LNP during formation to provide membrane integrity and stability of the lipid particle. Non-cationic lipids include amphipathic lipids, neutral lipids and anionic lipids. Accordingly, the non-cationic lipid can be a neutral uncharged, zwitterionic, or anionic lipid.

Exemplary non-cationic lipids include, but are not limited to, phospholipids such as distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-phosphoethanolamine (DLPE); 1,2-diphytanoyl-sn-glycero-3-phosphoethanolamine (DPHyPE); lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is to be understood that other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, palmitoyl, stearoyl, or oleoyl. In one embodiment of any of the aspects or embodiments herein, the non-cationic lipid is any one or more selected from dioleoylphosphatidylcholine (DOPC), distearoylphosphatidylcholine (DSPC), and dioleoyl-phosphatidylethanolamine (DOPE).

Other examples of non-cationic lipids suitable for use in the lipid particles (e.g., lipid nanoparticles) include nonphosphorous lipids such as, e.g., stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, sphingomyelin, and the like.

Additional exemplary non-cationic lipids are described in International Patent Application Publication No. WO2017/099823 and U.S. Patent Application Publication No. US2018/0028664, the contents of both of which are incorporated herein by reference in their entireties.

In one embodiment of any of the aspects or embodiments herein, in a lipid nanoparticle, the non-cationic lipid is present at a molar percentage of about 2% to about 20%, e.g., about 3% to about 20%, about 5% to about 20%, about 7% to about 20%, about 8% to about 20%, about 10% to about 20%, about 12% to about 20%, about 13% to about 20%, about 15% to about 20%, about 17% to about 20%, about 18% to about 20%, about 2% to about 18%, about 3% to about 18%, about 5% to about 18%, about 7% to about 18%, about 8% to about 18%, about 10% to about 18%, about 12% to about 18%, about 13% to about 18%, about 15% to about 18%, about 17% to about 18%, about 2% to about 17%, about 3% to about 17%, about 5% to about 17%, about 7% to about 17%, about 8% to about 17%, about 10% to about 17%, about 12% to about 17%, about 13% to about 17%, about 15% to about 17%, about 2% to about 15%, about 3% to about 15%, about 5% to about 15%, about 7% to about 15%, about 8% to about 15%, about 10% to about 15%, about 12% to about 15%, about 13% to about 15%, about 2% to about 13%, about 3% to about 13%, about 5% to about 13%, about 7% to about 13%, about 8% to about 13%, about 10% to about 13%, about 12% to about 13%, about 2% to about 12%, about 3% to about 12%, about 5% to about 12%, about 7% to about 12%, about 8% to about 12%, about 10% to about 12%, about 2% to about 10%, about 3% to about 10%, about 5% to about 10%, about 7% to about 10%, about 8% to about 10%, about 2% to about 8%, about 3% to about 8%, about 5% to about 8%, about 7% to about 8%, about 2% to about 7%, about 3% to about 7%, about 5% to about 7%, about 2% to about 5%, about 3% to about 5%, or about 2% to about 3%. In one embodiment of any of the aspects or embodiments herein, in a lipid nanoparticle, the non-cationic lipid is present at a molar percentage of about 5% to about 15%, about 7% to about 15%, about 8% to about 15%, about 10% to about 15%, about 12% to about 15%, about 13% to about 15%, 5% to about 13%, about 7% to about 13%, about 8% to about 13%, about 10% to about 13%, about 12% to about 13%, about 5% to about 12%, about 7% to about 12%, about 8% to about 12%, about 10% to about 12%, about 5% to about 10%, about 7% to about 10%, about 8% to about 10%, about 5% to about 8%, about 7% to about 8%, or about 5% to about 7%; such as but not limited to about 5%, about 6%, about 7%, about 8%, about 9%, about 10%, about 11%, about 11%, about 12%, about 13%, about 14%, or about 15%.

### PEGylated Lipids

In one embodiment of any of the aspects or embodiments herein, a lipid nanoparticle (LNP) described herein further comprises at least one PEGylated lipid (e.g., one, two, or three). A PEGylated lipid is a lipid as defined herein that is covalently or non-covalently linked to one or more polyethylene glycol (PEG) polymer chains, and is therefore a class of conjugated lipids. Generally, PEGylated lipids are incorporated in LNPs to inhibit aggregation of the particle and/or provide steric stabilization. In one embodiment of any of the aspects or embodiments herein, the lipid is covalently linked to the one or more PEG polymer chains.

Suitable PEG molecules for use in a PEGylated lipid include but are not limited to those having a molecular weight of between about 500 and about 10,000, or between about 1,000 and about 7,500, or about between about 1,000 and about 5,000, or between about 2,000 and about 5,000, or between about 2,000 and about 4,000, or between about 2,000 and about 3,500, or between about 2,000 and about 3,000; e.g., PEG2000, PEG2500, PEG3000, PEG3350, PEG3500, and PEG4000.

The lipid to which the one or more PEG chains are linked to can be a sterol, a non-cationic lipid, or a phospholipid. Exemplary PEGylated lipids include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a PEGylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, or a mixture thereof. Additional exemplary PEGylated lipids are described, for example, in U.S. Pat. Nos. 5,885,613 and 6,287,591 and U.S. Patent Application Publication Nos. US2003/0077829, US2003/0077829, US2005/0175682, US2008/0020058, US2011/0117125, US2010/0130588, US2016/0376224, and US2017/0119904, the contents of all of which are incorporated herein by reference in their entirety.

In one embodiment of any of the aspects or embodiments herein, the at least one PEGylated lipid in a lipid nanoparticle (LNP) provided herein is selected from the group consisting of PEG-dilauryloxypropyl; PEG-dimyristyloxypropyl; PEG-dipalmitoylglycerol, PEG-distearyloxypropyl; 1-(monomethoxy-polyethyleneglycol)-2,3-dimyristoylglycerol-PEG (DMG-PEG); distearoyl-rac-glycerol-PEG (DSG-PEG); PEG-dilaurylglycerol; PEG-dipalmitoylglycerol; PEG-disterylglycerol; PEG-dilaurylglycamide; PEG-dimyristylglycamide: PEG-dipalmitoylglycamide; PEG-disterylglycamide: (1-[8′-(Cholest-5-en-3[beta]-oxy)carboxamido-3′,6′-dioxaoctanyl]carbamoyl-[omega]-methyl-poly(ethylene glycol) (PEG-cholesterol); 3,4-ditetradecoxylbenzyl-[omega]-methyl-poly(ethylene glycol) ether (PEG-DMB), 1,2-dimyristoyl-sn-glycero-3-phosphoethanolamine-N-[methoxy(polyethylene glycol) (DSPE-PEG), and 1,2-distearoyl-sn-glycero-3-phosphoethanolamine-N-poly(ethylene glycol)-hydroxyl (DSPE-PEG-OH). In one embodiment of any of the aspects or embodiments herein, the at least one PEGylated lipid is DMG-PEG, DSPE-PEG, DSPE-PEG-OH, DSG-PEG, or a combination thereof. In one embodiment of any of the aspects or embodiments herein, the at least one PEGylated lipid is DMG-PEG2000, DSPE-PEG2000, DSPE-PEG2000-OH, DSG-PEG2000, or a combination thereof. In one embodiment of any of the aspects or embodiments herein, a lipid nanoparticle (LNP) provided herein comprises DMG-PEG2000 and DSPE-PEG2000. In one embodiment of any of the aspects or embodiments herein, a lipid nanoparticle (LNP) provided herein comprises DMG-PEG2000 and DSG-PEG2000. In one embodiment of any of the aspects or embodiments herein, a lipid nanoparticle (LNP) provided herein comprises DSPE-PEG2000 and DSPE-PEG2000-OH.

In one embodiment of any of the aspects or embodiments herein, in a lipid nanoparticle, the at least one PEGylated lipid is present, in total, at a molar percentage of about 1% to 10%, e.g., about 1.5% to about 10%, about 2% to about 10%, about 2.5% to about 10%, about 3% to about 10%, about 3.5% to about 10%, about 4% to about 10%, about 4.5% to about 10%, about 5% to about 10%, about 5.5% to about 10%, about 6% to about 10%, about 6.5% to about 10%, about 7% to about 10%, about 7.5% to about 10%, about 8% to about 10%, about 8.5% to about 10%, about 9% to about 10%, about 9.5% to about 10%, about 1% to about 5%, about 1.5% to about 5%, about 2% to about 5%, about 2.5% to about 5%, about 3% to about 5%, about 3.5% to about 5%, about 4% to about 5%, about 4.5% to about 5%, about 1% to about 4%, about 1.5% to about 4%, about 2% to about 4%, about 2.5% to about 4%, about 3% to about 4%, about 3.5% to about 4%, about 1% to about 3.5%, about 1.5% to about 3.5%, about 2% to about 3.5%, about 2.5% to about 3.5%, about 3% to about 3.5%, about 1% to about 3%, about 1.5% to about 3%, about 2% to about 3%, about 2.5% to about 3%, about 1% to about 2.5%, about 1.5% to about 2.5%, about 2% to about 2.5%, about 1% to about 2%, about 1.5% to about 2%, or about 1% to about 1.5%. In one embodiment of any of the aspects or embodiments herein, in a lipid nanoparticle, the at least one PEGylated lipid is present, in total, at a molar percentage of about 1% to about 2%, about 1.5% to about 2%, or about I % to about 1.5%: such as but not limited to about 1%, about 1.1%, about 1.2%, about 1.3%, about 1.4%, about 1.5%, about 1.6%, about 1.7%, about 1.8%, about 1.9%, or about 2%.

In one embodiment of any of the aspects or embodiments herein, in a lipid nanoparticle, the at least one PEGylated lipid is present, in total, at a molar percentage of about 2.1% to about 10%, e.g., about 2.5% to about 10%, about 3% to about 10%, about 3.5% to about 10%, about 4% to about 10%, about 4.5% to about 10%, about 5% to about 10%, about 5.5% to about 10%, about 6% to about 10%, about 6.5% to about 10%, about 7% to about 10%, about 7.5% to about 10%, about 8% to about 10%, about 8.5% to about 10%, about 9% to about 10%, about 9.5% to about 10%, about 2.1% to about 7%, about 2.5% to about 7%, about 3% to about 7%, about 3.5% to about 7%, about 4% to about 7%, about 4.5% to about 7%, about 5% to about 7%, about 5.5% to about 7%, about 6% to about 7%, about 6.5% to about 7%, about 2.1% to about 5%, about 2.5% to about 5%, about 3% to about 5%, about 3.5% to about 5%, about 4% to about 5%, about 4.5% to about 5%, about 2.1% to about 4%, about 2.5% to about 4%, about 3% to about 4%, about 3.5% to about 4%, about 2.1% to about 3.5%, about 2.5% to about 3.5%, about 3% to about 3.5%, about 2.1% to about 3%, about 2.5% to about 3%, or about 2.1% to about 2.5%. In one embodiment of any of the aspects or embodiments herein, in a lipid nanoparticle, the at least one PEGylated lipid is present, in total, at a molar percentage of about 2.1% to about 5%, about 2.5% to about 5%, about 3% to about 5%, about 3.5% to about 5%, about 4% to about 5%, about 4.5% to about 5%, about 2.1% to about 4%, about 2.5% to about 4%, about 3% to about 4%, about 3.5% to about 4%, about 2.1% to about 3.5%, about 2.5% to about 3.5%, about 3% to about 3.5%, about 2.1% to about 3%, about 2.5% to about 3%, or about 2.1% to about 2.5%: such as but not limited to about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, or about 5%.

### Tissue-Specific Targeting Ligands and PEGylated Lipid Conjugates

In one embodiment of any of the aspects or embodiments herein, a lipid nanoparticle (LNP) described herein further comprises at least one tissue-specific targeting ligand for the purpose of aiding, enhancing and/or increasing the delivery of the LNP to a target site of interest. The ligand may be any biological molecule such as a peptide, a protein, an antibody, a glycan, a sugar, a nucleic acid, a lipid or a conjugate comprising any of the foregoing, that recognizes a receptor or a surface antigen that is unique to certain cells and tissues.

In one embodiment of any of the aspects or embodiments herein, the at least one tissue-specific targeting ligand is N-Acetylgalactosamine (GalNAc) or a GalNAc derivative. The term “GalNAc derivative” encompasses modified GalNAc, functionalized GalNAc, and GalNAc conjugates wherein one or more GalNAc molecules (native or modified) is covalently linked to one or more functional groups or one or more classes of exemplary biological molecules such as but not limited to a peptide, a protein, an antibody, a glycan, a sugar, a nucleic acid, a lipid). The biological molecule itself, to which the one or more GalNAc molecules may be conjugated to, typically help to increase the stability and/or to inhibit aggregation. In one embodiment of any of the aspects or embodiments herein, the mol ratio between a tissue-specific target ligand, such as GalNAc, and the biological molecule to which the ligand is conjugated to is 1:1, 2:1, 3:1, 4:1, 5:1, 6:1, 7:1, 8:1, 9:1, 10:1, 1:2, 1:3, 1:4, 1:5, 1:6, 1:7, 1:8, 1:9, and 1:10. In one embodiment of any of the aspects or embodiments herein, the mol ratio between a tissue-specific target ligand, such as GalNAc, and the biological molecule to which the ligand is conjugated to is 1:1 (e.g., mono-antennary GalNAc), 2:1 (bi-antennary GalNAc), 3:1 (tri-antennary GalNAc), and 4:1 (tetra-antennary GalNAc). Conjugated GalNAc such as tri-antennary GalNAc (GalNAc3) or tetra-antennary GalNAc (GalNAc4) can be synthesized as known in the art (see, WO2017/084987 and WO2013/166121) and chemically conjugated to lipid or PEG as well-known in the art (see, Resen et al., J. Biol. Chem. (2001) “Determination of the Upper Size Limit for Uptake and Processing of Ligands by the Asialoglycoprotein Receptor on Hepatocytes in Vitro and in Vivo” 276:375577-37584).

In one embodiment of any of the aspects or embodiments herein, the tissue-specific targeting ligand is covalently linked to a PEGylated lipid as defined and described herein to form a PEGylated lipid conjugate. Exemplary PEGylated lipids are described above, and include PEG-dilauryloxypropyl; PEG-dimyristyloxypropyl; PEG-dipalmityloxypropyl, PEG-distearyloxypropyl; 1-(monomethoxy-polyethyleneglycol)-2,3-dimyristoylglycerol (DMG-PEG); PEG-dilaurylglycerol; PEG-dipalmitoylglycerol; PEG-disterylglycerol; PEG-dilaurylglycamide; PEG-dimyristylglycamide; PEG-dipalmitoylglycamide; PEG-disterylglycamide; (1-[8′-(Cholest-5-en-3[beta]-oxy)carboxamido-3′,6′-dioxaoctanyl]carbamoyl-[omega]-methyl-poly(ethylene glycol) (PEG-cholesterol); 3,4-ditetradecoxylbenzyl-[omega]-methyl-poly(ethylene glycol) ether (PEG-DMB); 1,2-dimyristoyl-sn-glycero-3-phosphoethanolamine-N-[methoxy(polyethylene glycol) (DSPE-PEG); and 1,2-distearoyl-sn-glycero-3-phosphoethanolamine-N-poly(ethylene glycol)-hydroxyl (DSPE-PEG-OH). In one embodiment of any of the aspects or embodiments herein, a lipid nanoparticle (LNP) provided herein comprises DMG-PEG2000 and DSPE-PEG2000. In one embodiment of any of the aspects or embodiments herein, the tissue-specific targeting ligand is covalently linked to GalNAc or a GalNAc derivative. In one embodiment of any of the aspects or embodiments herein, the PEGylated lipid conjugate is mono-, bi-, tri-, or tetra-antennary GalNAc-DSPE-PEG. In one embodiment of any of the aspects or embodiments herein, the PEGylated lipid conjugate is mono-, bi-, tri-, or tetra-antennary GalNAc-DSG-PEG. In one embodiment of any of the aspects or embodiments herein, the PEGylated lipid conjugate is mono-, bi-, tri-, or tetra-antennary GalNAc-DSPE-PEG2000. In one embodiment of any of the aspects or embodiments herein, the PEGylated lipid conjugate is mono-, bi-, tri-, or tetra-antennary GalNAc-DSG-PEG2000. In one embodiment of any of the aspects or embodiments herein, the PEGylated lipid conjugate is tri-antennary GalNAc-DSPE-PEG2000. In one embodiment of any of the aspects or embodiments herein, the PEGylated lipid conjugate is tri-antennary GalNAc-DSG-PEG2000. In one embodiment of any of the aspects or embodiments herein, the PEGylated lipid conjugate is tetra-antennary GalNAc-DSPE-PEG2000. In one embodiment of any of the aspects or embodiments herein, the PEGylated lipid conjugate is tetra-antennary GalNAc-DSG-PEG2000.

In one embodiment of any of the aspects or embodiments herein, in a lipid nanoparticle, the PEGylated lipid conjugate is present at a molar percentage of about 0.1% to about 10%, e.g., about 0.2% to about 10%, about 0.3% to about 10%, about 0.4% to about 10%, about 0.5% to about 10%, about 0.6% to about 10%, about 0.7% to about 10%, about 0.8% to about 10%, about 0.9% to about 10%, about 1% to about 10%, about 1.5% to about 10%, about 2% to about 10%, about 2.5% to about 10%, about 3% to about 10%, about 3.5% to about 10%, about 4% to about 10%, about 4.5% to about 10%, about 5% to about 10%, about 5.5% to about 10%, about 6% to about 10%, about 6.5% to about 10%, about 7% to about 10%, about 7.5% to about 10%, about 8% to about 10%, about 8.5% to about 10%, about 9% to about 10%, about 9.5% to about 10%, about 0.1% to about 5%, about 0.2% to about 5%, about 0.3% to about 5%, about 0.4% to about 5%, about 0.5% to about 5%, about 0.6% to about 5%, about 0.7% to about 5%, about 0.8% to about 5%, about 0.9% to about 10%, about 1% to about 5%, about 1.5% to about 5%, about 2% to about 5%, about 2.5% to about 5%, about 3% to about 5%, about 3.5% to about 5%, about 4% to about 5%, about 4.5% to about 5%, about 0.1% to about 3%, about 0.2% to about 3%, about 0.3% to about 3%, about 0.4% to about 3%, about 0.5% to about 3%, about 0.6% to about 3%, about 0.7% to about 3%, about 0.8% to about 3%, about 0.9% to about 3%, about 1% to about 3%, about 1.5% to about 3%, about 2% to about 3%, about 2.5% to about 3%, about 0.1% to about 2%, about 0.2% to about 2%, about 0.3% to about 2%, about 0.4% to about 2%, about 0.5% to about 2%, about 0.6% to about 2%, about 0.7% to about 2%, about 0.8% to about 2%, about 0.9% to about 2%, about 1% to about 2%, about 1.5% to about 2%, about 0.1% to about 1.5%, 0.2% to about 1.5%, about 0.3% to about 1.5%, about 0.4% to about 1.5%, about 0.5% to about 1.5%, about 0.6% to about 1.5%, about 0.7% to about 1.5%, about 0.8% to about 1.5%, about 0.9% to about 1.5%, about 1% to about 1.5%, about 0.1% to about 1%, 0.2% to about 1%, about 0.3% to about 1%, about 0.4% to about 1%, about 0.5% to about 1%, about 0.6% to about 1%, about 0.7% to about 1%, about 0.8% to about 1%, or about 0.9% to about 1%. In one embodiment of any of the aspects or embodiments herein, in a lipid nanoparticle, the PEGylated lipid conjugate is present at a molar percentage of about 0.1% to about 1.5%, about 0.2% to about 1.5%, about 0.3% to about 1.5%, about 0.4% to about 1.5%, about 0.5% to about 1.5%, about 0.6% to about 1.5%, about 0.7% to about 1.5%, about 0.8% to about 1.5%, about 0.9% to about 1.5%, about 1% to about 1.5%, about 0.1% to about 1%, about 0.2% to about 1%, about 0.3% to about 1%, about 0.4% to about 1%, about 0.5% to about 1%, about 0.6% to about 1%, about 0.7% to about 1%, about 0.8% to about 1%, or about 0.9% to about 1%.; such as but not limited to about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, or about 1.5%.

### Other Components of Lipid Nanoparticles (LNP)

Additional components of LNP such as conjugated lipids are also contemplated in this disclosure. Exemplary conjugated lipids include, but are not limited to, polyoxazoline (POZ)-lipid conjugates, polyamide-lipid conjugates (such as ATIA-lipid conjugates), cationic-polymer lipid (CPL) conjugates, and mixtures thereof.

Furthermore, in one embodiment of any of the aspects or embodiments herein, a lipid nanoparticle (LNP) described herein further comprises, for example, by co-encapsulation within the LNP or by conjugation to any one of the components of the LNP as described above, an immune-modulating compound. The immune-modulating compound, such as dexamethasone or a modified dexamethasone, may aid in of minimizing immune response. In one embodiment of any of the aspects or embodiments herein, a lipid nanoparticle (LNP) described herein further comprises dexamethasone palmitate.

In some embodiments of any of the aspects and embodiments herein, in addition to the cationic lipid, the lipid nanoparticle comprises an agent for condensing and/or encapsulating nucleic acid cargo, such as ceDNA. Such an agent is also referred to as a condensing or encapsulating agent herein. Without limitations, any compound known in the art for condensing and/or encapsulating nucleic acids can be used as long as it is non-fusogenic. In other words, an agent capable of condensing and/or encapsulating the nucleic acid cargo, such as ceDNA, but having little or no fusogenic activity. Without wishing to be bound by a theory, a condensing agent may have some fusogenic activity when not condensing/encapsulating a nucleic acid, such as ceDNA, but a nucleic acid encapsulating lipid nanoparticle formed with said condensing agent can be non-fusogenic.

### Total Lipid to Nucleic Acid Ratio

Generally, the lipid particles (e.g., lipid nanoparticles) are prepared such that the final particle has a total lipid to cleavable ceDNA (mass or weight) ratio of from about 10:1 to 60:1, e.g., about 15:1 to about 60:1, about 20:1 to about 60:1, about 25:1 to about 60:1, about 30:1 to about 60:1, about 35:1 to about 60:1, about 40:1 to about 60:1, about 45:1 to about 60:1, about 50:1 to about 60:1, about 55:1 to about 60:1, about 10:1 to about 55:1, about 15:1 to about 55:1, about 20:1 to about 55:1, about 25:1 to about 55:1, about 30:1 to about 55:1, about 35:1 to about 55:1, about 40:1 to about 55:1, about 45:1 to about 55:1, about 50:1 to about 55:1, about 10:1 to about 50:1, about 15:1 to about 50:1, about 20:1 to about 50:1, about 25:1 to about 50:1, about 3:1 to about 50:1, about 35:1 to about 50:1, about 40:1 to about 50:1, about 45:1 to about 50:1, about 10:1 to about 45:1, about 15:1 to about 45:1, about 20:1 to about 45:1, about 25:1 to about 45:1, about 30-1 to about 45:1, about 35:1 to about 45:1, about 40:1 to about 45:1, about 10:1 to about 40:1, about 15:1 to about 40:1, about 20:1 to about 40:1, about 25:1 to about 40:1, about 30:1 to about 40:1, about 35:1 to about 40:1, about 10:1 to about 35:1, about 15:1 to about 35:1, about 20:1 to about 35:1, about 25:1 to about 35:1, about 30:1 to about 35:1, about 10:1 to about 30:1, about 15:1 to about 30:1, about 20-1 to about 30:1, about 25:1 to about 30:1, about 10:1 to about 25:1, about 15:1 to about 25:1, about 20-1 to about 25:1, about 10:1 to about 20:1, about 15:1 to about 20:1, or about 10:1 to about 15:1.

The amounts of lipids and nucleic acid can be adjusted to provide a desired N/P ratio (i.e., ratio of positively-chargeable polymer amine (N=nitrogen) groups to negatively-charged nucleic acid phosphate (P) groups), for example, an N/P ratio of 3, 4, 5, 6, 7, 8, 9, 10, 11, 12, 13, 14 15, 16, 17, 18, 19, 20, 21, 22, 23, 24, 25 or higher. Generally, the lipid particle formulation's overall lipid content can range from about 5 mg/ml to about 30 mg/mL.

### Size of Lipid Nanoparticles (LNP)

According to some embodiments of any of the aspects or embodiments herein, the LNP has a diameter ranging from about 40 nm to about 120 nm, e.g., about 45 nm to about 120 nm, about 50 nm to about 120 nm, about 55 nm to about 120 nm, about 60 nm to about 120 nm, about 65 nm to about 120 nm, about 70 nm to about 120 nm, about 75 nm to about 120 nm, about 80 nm to about 120 nm, about 85 nm to about 120 nm, about 90 nm to about 120 nm, about 95 nm to about 120 nm, about 100 nm to about 120 nm, about 105 nm to about 120 nm, about 110 nm to about 120 nm, about 115 nm to about 120 nm, about 40 nm to about 110 nm, about 45 nm to about 110 nm, about 50 nm to about 110 nm, about 55 nm to about 110 nm, about 60 nm to about 110 nm, about 65 nm to about 110 nm, about 70 nm to about 110 nm, about 75 nm to about 110 nm, about 80 nm to about 110 nm, about 85 nm to about 110 nm, about 90 nm to about 110 nm, about 95 nm to about 110 nm, about 100 nm to about 110 nm, about 105 nm to about 110 nm, about 40 nm to about 100 nm, about 45 nm to about 100 nm, about 50 nm to about 100 nm, about 55 nm to about 100 nm, about 60 nm to about 100 nm, about 65 nm to about 100 nm, about 70 nm to about 100 nm, about 75 nm to about 100 nm, about 80 nm to about 100 nm, about 85 nm to about 100 nm, about 90 nm to about 100 nm, or about 95 nm to about 100 nm.

According to some embodiments of any of the aspects or embodiments herein, the LNP has a diameter of less than about 100 nm, e.g., about 40 nm to about 90 nm, about 45 nm to about 90 nm, about 50 nm to about 90 nm, about 55 nm to about 90 nm, about 60 nm to about 90 nm, about 65 nm to about 90 nm, about 70 nm to about 90 nm, about 75 nm to about 90 nm, about 80 nm to about 90 nm, about 85 nm to about 90 nm, about 40 nm to about 85 nm, about 45 nm to about 85 nm, about 50 nm to about 85 nm, about 55 nm to about 85 nm, about 60 nm to about 85 nm, about 65 nm to about 85 nm, about 70 nm to about 85 nm, about 75 nm to about 85 nm, about 80 nm to about 85 nm, about 40 nm to about 80 nm, about 45 nm to about 80 nm, about 50 nm to about 80 nm, about 55 nm to about 80 nm, about 60 nm to about 80 nm, about 65 nm to about 80 nm, about 70 nm to about 80 nm, about 75 nm to about 80 nm, about 40 nm to about 75 nm, about 45 nm to about 75 nm, about 50 nm to about 75 nm, about 55 nm to about 75 nm, about 60 nm to about 75 nm, about 65 nm to about 75 nm, about 70 nm to about 75 nm, about 40 nm to about 70 nm, about 45 nm to about 70 nm, about 50 nm to about 70 nm, about 55 nm to about 70 nm, about 60 nm to about 70 nm, or about 65 nm to about 70 nm. In one embodiment of any of the aspects or embodiments herein, the LNP has a diameter of about 60 nm to about 85 nm, about 65 nm to about 85 nm, about 70 nm to about 85 nm, about 75 nm to about 85 nm, about 80 nm to about 85 nm, about 60 nm to about 80 nm, about 65 nm to about 80 nm, about 70 nm to about 80 nm, about 75 nm to about 80 nm, about 60 nm to about 75 nm, about 65 nm to about 75 nm, about 70 nm to about 75 nm, about 60 nm to about 70 nm, or about 65 nm to about 70 nm; such as but not limited to about 60 mm, about 61 mm, about 62 mm, about 63 mm, about 64 mm, about 65 mm, about 66 mm, about 67 mm, about 68 mm, about 69 mm, about 70 mm, about 71 mm, about 72 mm, about 73 mm, about 74 mm, about 75 mm, about 76 mm, about 77 mm, about 78 mm, about 79 mm, about 80 mm, about 81 mm, about 82 mm, about 83 mm, about 84 mm, or about 85 mm.

In one embodiment of any of the aspects or embodiments herein, lipid particle (e.g., lipid nanoparticle) size can be determined by quasi-elastic light scattering using, for example, a Malvern Zetasizer Nano ZS (Malvern, UK) system.

### C. Conjugates

In some embodiments, a cleavable ceDNA vector as disclosed herein is conjugated (e.g., covalently bound to an agent that increases cellular uptake. An “agent that increases cellular uptake” is a molecule that facilitates transport of a nucleic acid across a lipid membrane. For example, a nucleic acid can be conjugated to a lipophilic compound (e.g., cholesterol, tocopherol, etc.), a cell penetrating peptide (CPP) (e.g., penetratin. TAT, Syn1B, etc.), and polyamines (e.g., spermine). Further examples of agents that increase cellular uptake are disclosed, for example, in Winkler (2013). Oligonucleotide conjugates for therapeutic applications. Ther. Deliv. 4(7); 791-809.

In some embodiments, a cleavable ceDNA vector as disclosed herein is conjugated to a polymer (e.g., a polymeric molecule) or a folate molecule (e.g., folic acid molecule). Generally, delivery of nucleic acids conjugated to polymers is known in the art, for example as described in WO2000/34343 and WO2008/022309. In some embodiments, a cleavable ceDNA vector as disclosed herein is conjugated to a poly(amide) polymer, for example as described by U.S. Pat. No. 8,987,377. In some embodiments, a nucleic acid described by the disclosure is conjugated to a folic acid molecule as described in U.S. Pat. No. 8,507,455.

In some embodiments, a cleavable ceDNA vector as disclosed herein is conjugated to a carbohydrate, for example as described in U.S. Pat. No. 8,450,467.

### D. Nanocapsule

Alternatively, nanocapsule formulations of a cleavable ceDNA vector as disclosed herein can be used. Nanocapsules can generally entrap substances in a stable and reproducible way. To avoid side effects due to intracellular polymeric overloading, such ultrafine particles (sized around 0.1 tm) should be designed using polymers able to be degraded in vivo. Biodegradable polyalkyl-cyanoacrylate nanoparticles that meet these requirements are contemplated for use.

### E. Liposomes

The cleavable ceDNA vectors in accordance with the present invention can be added to liposomes for delivery to a cell or target organ in a subject. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

The formation and use of liposomes is generally known to those of skill in the art. Liposomes have been developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 ANG, containing an aqueous solution in the core.

In some embodiments, a liposome comprises cationic lipids. The term “cationic lipid” includes lipids and synthetic lipids having both polar and non-polar domains and which are capable of being positively charged at or around physiological pH and which bind to polyanions, such as nucleic acids, and facilitate the delivery of nucleic acids into cells. In some embodiments, cationic lipids include saturated and unsaturated alkyl and alicyclic ethers and esters of amines, amides, or derivatives thereof. In some embodiments, cationic lipids comprise straight-chain, branched alkyl, alkenyl groups, or any combination of the foregoing. In some embodiments, cationic lipids contain from 1 to about 25 carbon atoms (e.g., 1, 2, 3, 4, 5, 6, 7, 8, 9, 10, 11, 12, 13, 14, 15, 16, 17, 18, 19, 20, 21, 22, 23, 24, or 25 carbon atoms. In some embodiments, cationic lipids contain more than 25 carbon atoms. In some embodiments, straight chain or branched alkyl or alkene groups have six or more carbon atoms. A cationic lipid can also comprise, in some embodiments, one or more alicyclic groups. Non-limiting examples of alicyclic groups include cholesterol and other steroid groups. In some embodiments, cationic lipids are prepared with a one or more counterions. Examples of counterions (anions) include but are not limited to Cl⁻, Br⁻, I⁻, F⁻, acetate, trifluoroacetate, sulfate, nitrite, and nitrate.

In some aspects, the disclosure provides for a liposome formulation that includes one or more compounds with a polyethylene glycol (PEG) functional group (so-called “PEG-ylated compounds”) which can reduce the immunogenicity/antigenicity of, provide hydrophilicity and hydrophobicity to the compound(s) and reduce dosage frequency. Or the liposome formulation simply includes polyethylene glycol (PEG) polymer as an additional component. In such aspects, the molecular weight of the PEG or PEG functional group can be from62 Da to about 5,000 Da.

In some aspects, the disclosure provides for a liposome formulation that will deliver an API with extended release or controlled release profile over a period of hours to weeks. In some related aspects, the liposome formulation may comprise aqueous chambers that are bound by lipid bilayers. In other related aspects, the liposome formulation encapsulates an API with components that undergo a physical transition at elevated temperature which releases the API over a period of hours to weeks.

In some aspects, the liposome formulation comprises sphingomyelin and one or more lipids disclosed herein. In some aspects, the liposome formulation comprises optisomes.

In some aspects, the disclosure provides for a liposome formulation that includes one or more lipids selected from: N-(carbonyl-methoxypolyethylene glycol 2000)-1,2-distearoyl-sn-glycero-3-phosphoethanolamine sodium salt, (distearoyl-sn-glycero-phosphoethanolamine), MPEG (methoxy polyethylene glycol)-conjugated lipid, HSPC (hydrogenated soy phosphatidylcholine); PEG (polyethylene glycol); DSPE (distearoyl-sn-glycero-phosphoethanolamine); DSPC (distearoylphosphatidylcholine); DOPC (dioleoylphosphatidylcholine); DPPG (dipalmitoylphosphatidylglycerol); EPC (egg phosphatidylcholine); DOPS (dioleoylphosphatidylserine); POPC (palmitoyloleoylphosphatidylcholine); SM (sphingomyelin); MPEG (methoxy polyethylene glycol); DMPC (dimyristoyl phosphatidylcholine); DMPG (dimyristoyl phosphatidylglycerol); DSPG (distearoylphosphatidylglycerol); DEPC (dierucoylphosphatidylcholine); DOPE (dioleoly-sn-glycero-phophoethanolamine), cholesteryl sulphate (CS), dipalmitoylphosphatidylglycerol (DPPG), DOPC (dioleoly-sn-glycero-phosphatidylcholine) or any combination thereof.

In some aspects, the disclosure provides for a liposome formulation comprising phospholipid, cholesterol and a PEG-ylated lipid in a molar ratio of 56:38:5. In some aspects, the liposome formulation's overall lipid content is from 2-16 mg/mL. In some aspects, the disclosure provides for a liposome formulation comprising a lipid containing a phosphatidylcholine functional group, a lipid containing an ethanolamine functional group and a PEG-ylated lipid. In some aspects, the disclosure provides for a liposome formulation comprising a lipid containing a phosphatidylcholine functional group, a lipid containing an ethanolamine functional group and a PEG-ylated lipid in a molar ratio of 3:0.015:2 respectively. In some aspects, the disclosure provides for a liposome formulation comprising a lipid containing a phosphatidylcholine functional group, cholesterol and a PEG-ylated lipid. In some aspects, the disclosure provides for a liposome formulation comprising a lipid containing a phosphatidylcholine functional group and cholesterol. In some aspects, the PEG-ylated lipid is PEG-2000-DSPE. In some aspects, the disclosure provides for a liposome formulation comprising DPPG, soy PC, MPEG-DSPE lipid conjugate and cholesterol.

In some aspects, the disclosure provides for a liposome formulation comprising one or more lipids containing a phosphatidylcholine functional group and one or more lipids containing an ethanolamine functional group. In some aspects, the disclosure provides for a liposome formulation comprising one or more: lipids containing a phosphatidylcholine functional group, lipids containing an ethanolamine functional group, and sterols, e.g., cholesterol. In some aspects, the liposome formulation comprises DOPC/DEPC; and DOPE.

In some aspects, the disclosure provides for a liposome formulation further comprising one or more pharmaceutical excipients, e.g., sucrose and/or glycine.

In some aspects, the disclosure provides for a liposome formulation that is wither unilamellar or multilamellar in structure. In some aspects, the disclosure provides for a liposome formulation that comprises multi-vesicular particles and/or foam-based particles. In some aspects, the disclosure provides for a liposome formulation that are larger in relative size to common nanoparticles and about 150 to 250 nm in size. In some aspects, the liposome formulation is a lyophilized powder.

In some aspects, the disclosure provides for a liposome formulation that is made and loaded with ceDNA vectors disclosed or described herein, by adding a weak base to a mixture having the isolated ceDNA outside the liposome. This addition increases the pH outside the liposomes to approximately 7.3 and drives the API into the liposome. In some aspects, the disclosure provides for a liposome formulation having a pH that is acidic on the inside of the liposome. In such cases the inside of the liposome can be at pH 4-6.9, and more preferably pH 6.5. In other aspects, the disclosure provides for a liposome formulation made by using intra-liposomal drug stabilization technology. In such cases, polymeric or non-polymeric highly charged anions and intra-liposomal trapping agents are utilized, e.g., polyphosphate or sucrose octasulfate.

In other aspects, the disclosure provides for a liposome formulation comprising phospholipids, lecithin, phosphatidylcholine and phosphatidylethanolamine.

Non-limiting examples of cationic lipids include polyethylenimine, polyamidoamine (PAMAM) starburst dendrimers, Lipofectin (a combination of DOTMA and DOPE), Lipofectase, LIPOFECTAMINE™ (e.g., LIPOFECTAMINE™ 2000), DOPE, Cytofectin (Gilead Sciences, Foster City, Calif.), and Eufectins (JBL, San Luis Obispo, Calif.). Exemplary cationic liposomes can be made from N-[1-(2,3-dioleoloxy)-propyl]-N,N,N-trimethylammonium chloride (DOTMA), N-[1-(2,3-dioleoloxy)-propyl]-N,N,N-trimethylammonium methylsulfate (DOTAP), 3β-[N-(N′,N′-dimethylaminoethane)carbamoyl]cholesterol (DC-Chol), 2.3,-dioleyloxy-N-[2(sperminecarboxamido)ethyl]-N,N-dimethyl-1-propanaminium trifluoracetate (DOSPA), 1,2-dimyristyloxypropyl-3-dimethyl-hydroxyethyl ammonium bromide; and dimethyldioctadecylammonium bromide (DDAB). Nucleic acids (e.g., CELiD) can also be complexed with, e.g., poly (L-lysine) or avidin and lipids can, or cannot, be included in this mixture, e.g., steryl-poly (L-lysine).

In some embodiments, a cleavable ceDNA vector as disclosed herein is delivered using a cationic lipid described in U.S. Pat. No. 8,158,601, or a polyamine compound or lipid as described in U.S. Pat. No. 8,034,376.

### F. Exemplary Liposome and Lipid Nanoparticle (LNP) Compositions

The cleavable ceDNA vectors in accordance with the present invention can be added to liposomes alone or in combination with, e.g., a nuclease and a gRNA, for delivery to a cell in need of gene editing, e.g., in need of a corrected sequence. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

In some aspects, the disclosure provides for a liposome formulation that includes one or more compounds with a polyethylene glycol (PEG) functional group (so-called “PEG-ylated compounds”) which can reduce the immunogenicity/antigenicity of, provide hydrophilicity and hydrophobicity to the compound(s) and reduce dosage frequency. Or the liposome formulation simply includes polyethylene glycol (PEG) polymer as an additional component. In such aspects, the molecular weight of the PEG or PEG functional group can be from 62 Da to about 5,000 Da.

In some aspects, the disclosure provides for a liposome formulation that will deliver an API with extended release or controlled release profile over a period of hours to weeks. In some related aspects, the liposome formulation may comprise aqueous chambers that are bound by lipid bilayers. In other related aspects, the liposome formulation encapsulates an API with components that undergo a physical transition at elevated temperature which releases the API over a period of hours to weeks.

In some aspects, the liposome formulation comprises sphingomyelin and one or more lipids disclosed herein. In some aspects, the liposome formulation comprises optisomes.

In some aspects, the disclosure provides for a liposome formulation that includes one or more lipids selected from: N-(carbonyl-methoxypolyethylene glycol 2000)-1,2-distearoyl-sn-glycero-3-phosphoethanolamine sodium salt, (distearoyl-sn-glycero-phosphoethanolamine), MPEG (methoxy polyethylene glycol)-conjugated lipid, HSPC (hydrogenated soy phosphatidylcholine); PEG (polyethylene glycol); DSPE (distearoyl-sn-glycero-phosphoethanolamine); DSPC (distearoylphosphatidylcholine); DOPC (dioleoylphosphatidylcholine); DPPG (dipalmitoylphosphatidylglycerol); EPC (egg phosphatidylcholine); DOPS (dioleoylphosphatidylserine); POPC (palmitoyloleoylphosphatidylcholine); SM (sphingomyelin); MPEG (methoxy polyethylene glycol); DMPC (dimyristoyl phosphatidylcholine); DMPG (dimyristoyl phosphatidylglycerol); DSPG (distearoylphosphatidylglycerol); DEPC (dierucoylphosphatidylcholine); DOPE (dioleoly-sn-glycero-phophoethanolamine), cholesteryl sulphate (CS), dipalmitoylphosphatidylglycerol (DPPG), DOPC (dioleoly-sn-glycero-phosphatidylcholine) or any combination thereof.

In some aspects, the disclosure provides for a liposome formulation comprising phospholipid, cholesterol and a PEG-ylated lipid in a molar ratio of 56:38:5. In some aspects, the liposome formulation's overall lipid content is from 2-16 mg/mL. In some aspects, the disclosure provides for a liposome formulation comprising a lipid containing a phosphatidylcholine functional group, a lipid containing an ethanolamine functional group and a PEG-ylated lipid. In some aspects, the disclosure provides for a liposome formulation comprising a lipid containing a phosphatidylcholine functional group, a lipid containing an ethanolamine functional group and a PEG-ylated lipid in a molar ratio of 3:0.015:2 respectively. In some aspects, the disclosure provides for a liposome formulation comprising a lipid containing a phosphatidylcholine functional group, cholesterol and a PEG-ylated lipid. In some aspects, the disclosure provides for a liposome formulation comprising a lipid containing a phosphatidylcholine functional group and cholesterol. In some aspects, the PEG-ylated lipid is PEG-2000-DSPE. In some aspects, the disclosure provides for a liposome formulation comprising DPPG, soy PC, MPEG-DSPE lipid conjugate and cholesterol.

In some aspects, the disclosure provides for a liposome formulation comprising one or more lipids containing a phosphatidylcholine functional group and one or more lipids containing an ethanolamine functional group. In some aspects, the disclosure provides for a liposome formulation comprising one or more: lipids containing a phosphatidylcholine functional group, lipids containing an ethanolamine functional group, and sterols, e.g., cholesterol. In some aspects, the liposome formulation comprises DOPC/DEPC; and DOPE.

In some aspects, the disclosure provides for a liposome formulation further comprising one or more pharmaceutical excipients, e.g., sucrose and/or glycine.

In some aspects, the disclosure provides for a liposome formulation that is either unilamellar or multilamellar in structure. In some aspects, the disclosure provides for a liposome formulation that comprises multi-vesicular particles and/or foam-based particles. In some aspects, the disclosure provides for a liposome formulation that are larger in relative size to common nanoparticles and about 150 to 250 nm in size. In some aspects, the liposome formulation is a lyophilized powder.

In some aspects, the disclosure provides for a liposome formulation that is made and loaded with ceDNA vectors disclosed or described herein, by adding a weak base to a mixture having the isolated ceDNA outside the liposome. This addition increases the pH outside the liposomes to approximately 7.3 and drives the API into the liposome. In some aspects, the disclosure provides for a liposome formulation having a pH that is acidic on the inside of the liposome. In such cases the inside of the liposome can be at pH 4-6.9, and more preferably pH 6.5. In other aspects, the disclosure provides for a liposome formulation made by using intra-liposomal drug stabilization technology. In such cases, polymeric or non-polymeric highly charged anions and intra-liposomal trapping agents are utilized, e.g., polyphosphate or sucrose octasulfate.

In other aspects, the disclosure provides for a liposome formulation comprising phospholipids, lecithin, phosphatidylcholine and phosphatidylethanolamine. In some embodiments, the liposomal formulation is a formulation described in the following Table 7.

TABLE 7
Exemplary liposomal formulations.

| Composition | pH | Composition | pH |
|---|---|---|---|
| MPEG-DSPE (3.19 mg/mL) | 6.5 | DSPC (28.16 mg/mL) | 4.9-6.0 |
| HSPC (9.58 mg/mL) | | Cholesterol (6.72 mg/mL) | |
| Cholesterol (3.19 mg/mL) | | | |
| DOPC (5.7 mg/mL) | 5.5-8.5 | Egg phosphatidylcholine:cholesterol | 7.8 |
| Cholesterol (4.4 mg/mL) | | (55:45 molar ratio)[reconstit. | |
| Triolein (1.2 mg/mL) | | from lyophilizate in | |
| DPPG (1.0 mg/mL) | | sodium carbonate buffer] | |
| DOPS:POPC (3:7 molar ratio) | 4.5-7.0 | Sphingomyelin (2.37 mg/mL, | 7.2-7.6 |
| 1 g total lipid/vial [reconstit. from | | 73.5 mg/31 mL) | |
| lyophilizate 0.9% NaCl] | | Cholesterol (0.95 mg/mL, | |
| | | 29.5 mg/31 mL) [reconstit. from | |
| | | lyophilizate in sodium phos. soln.] | |
| DSPC (6.81 mg/mL) | 6.8-7.6 | DMPC (3.4 mg/ml) | 5.0-7.0 |
| Cholesterol (2.22 mg/mL) | | DMPG (1.5 mg/ml) | |
| MPEG-2000-DSPE (0.12 mg/mL) | | in a 7:3 molar ratio | |
| HSPC (17.75 mg/mL, | 5.0-6.0 | Sodium cholesteryl sulfate (2.64 | |
| 213 mg/12 mL) | | mg/mL) [reconstit. from | |
| Cholesterol (4.33 mg/mL, | | lyophilizate in sterile water] | |
| 52 mg/12 mL) | | | |
| DSPG (7.0 mg/mL, | | | |
| 84 mg/12 mL) [reconstit. from | | | |
| lyophilizate in sterile water] | | | |
| DMPC and EPG | | DOPC (4.2 mg/mL) | 5.0-8.0 |
| (1:8 molar ratio) [reconstit. from | | Cholesterol (3.3 mg/mL) | |
| lyophilizate in sterile water] | | DPPG (0.9 mg/mL) | |
| | | Tricaprylin (0.3 mg/mL) | |
| | | Triolein (0.1 mg/mL) | |
| Cholesterol (4.7 mg/mL) | 5.8-7.4 | DOPC:DOPE | |
| DPPG (0.9 mg/mL) | | (75:25 molar ratio) | |
| Tricaprylin (2.0 mg/mL) | | | |
| DEPC (8.2 mg/mL) | | | |

In some aspects, the disclosure provides for a lipid nanoparticle comprising ceDNA and an ionizable lipid. For example, a lipid nanoparticle formulation that is made and loaded with ceDNA obtained by the process as disclosed in International Application PCT/US2018/050042, filed on Sep. 7, 2018, which is incorporated herein. This can be accomplished by high energy mixing of ethanolic lipids with aqueous ceDNA at low pH which protonates the ionizable lipid and provides favorable energetics for ceDNA/lipid association and nucleation of particles. The particles can be further stabilized through aqueous dilution and removal of the organic solvent. The particles can be concentrated to the desired level.

Generally, the lipid particles are prepared at a total lipid to ceDNA (mass or weight) ratio of from about 10:1 to 30-1. In some embodiments, the lipid to ceDNA ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and ceDNA can be adjusted to provide a desired N/P ratio, for example, N/P ratio of 3, 4, 5, 6, 7, 8, 9, 10 or higher. Generally, the lipid particle formulation's overall lipid content can range from about 5 mg/ml to about 30 mg/mL.

The ionizable lipid is typically employed to condense the nucleic acid cargo, e.g., ceDNA at low pH and to drive membrane association and fusogenicity. Generally, ionizable lipids are lipids comprising at least one amino group that is positively charged or becomes protonated under acidic conditions, for example at pH of 6.5 or lower. Ionizable lipids are also referred to as cationic lipids herein.

Exemplary ionizable lipids are described in PCT patent publications WO2015/095340, WO2015/199952, WO2018/011633, WO2017/049245, WO2015/061467, WO2012/040184, WO2012/000104, WO2015/074085, WO2016/081029, WO2017/004143, WO2017/075531, WO2017/117528, WO2011/022460, WO2013/148541, WO2013/116126, WO2011/153120, WO2012/044638, WO2012/054365, WO2011/090965, WO2013/016058, WO2012/162210, WO2008/042973, WO2010/129709, WO2010/144740, WO2012/099755, WO2013/049328, WO2013/086322, WO2013/086373, WO2011/071860, WO2009/132131, WO2010/048536, WO2010/088537, WO2010/054401, WO2010/054406, WO2010/054405, WO2010/054384, WO2012/016184, WO2009/086558, WO2010/042877, WO2011/000106, WO2011/000107, WO2005/120152, WO2011/141705, WO2013/126803, WO2006/007712, WO2011/038160, WO2005/121348, WO2011/066651, WO2009/127060, WO2011/141704, WO2006/069782, WO2012/031043, WO2013/006825, WO2013/033563, WO2013/089151, WO2017/099823, WO2015/095346, and WO2013/086354, and US patent publications US2016/0311759, US2015/0376115, US2016/0151284, US2017/0210697, US2015/0140070, US2013/0178541, US2013/0303587, US2015/0141678, US2015/0239926, US2016/0376224, US2017/0119904, US2012/0149894, US2015/0057373, US2013/0090372, US2013/0274523, US2013/0274504, US2013/0274504, US2009/0023673, US2012/0128760, US2010/0324120, US2014/0200257, US2015/0203446, US2018/0005363, US2014/0308304, US2013/0338210, US2012/0101148, US2012/0027796, US2012/0058144, US2013/0323269, US2011/0117125, US2011/0256175, US2012/0202871, US2011/0076335, US2006/0083780, US2013/0123338, US2015/0064242, US2006/0051405, US2013/0065939, US2006/0008910, US2003/0022649, US2010/0130588, US2013/0116307, US2010/0062967, US2013/0202684, US2014/0141070, US2014/0255472, US2014/0039032, US2018/0028664, US2016/0317458, and US2013/0195920, the contents of all of which are incorporated herein by reference in their entirety.

In some embodiments, the ionizable lipid is MC3 (6Z,9Z,28Z,31Z)-heptatriaconta-6,9,28,31-tetraen-19-yl-4-(dimethylamino) butanoate (DLin-MC3-DMA or MC3) having the following structure:

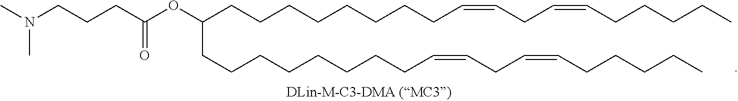 [SMILES: CCCCC/C=C\C/C=C\CCCCCCCCC(CCCCCCCC/C=C\C/C=C\CCCCC)OC(=O)CCCN(C)C]

The lipid DLin-MC3-DMA is described in Jayaraman et al., Angew. Chem. Int. Ed Engl. (2012), 51(34): 8529-8533, content of which is incorporated herein by reference in its entirety.

In some embodiments, the ionizable lipid is the lipid ATX-002 having the following structure:

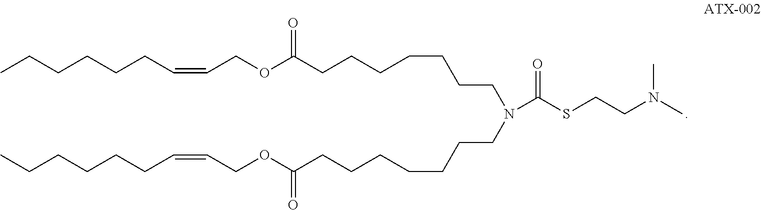 [SMILES: CCCCCC/C=C\COC(=O)CCCCCCCN(CCCCCCCC(=O)OC/C=C\CCCCCC)C(=O)SCCN(C)C]

The lipid ATX-002 is described in WO2015/074085, content of which is incorporated herein by reference in its entirety.

In some embodiments, the ionizable lipid is (13Z,16Z)-N,N-dimethyl-3-nonyldocosa-13,16-dien-1-amine (Compound 32) having the following structure:

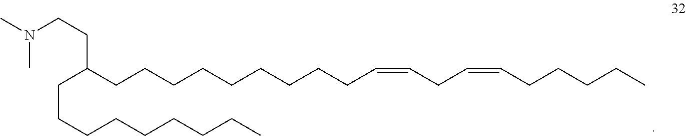 [SMILES: CCCCC/C=C\C/C=C\CCCCCCCCCC(CCCCCCCCC)CCN(C)C]

Compound 32 is described in WO2012/040184, content of which is incorporated herein by reference in its entirety.

In some embodiments, the ionizable lipid is Compound 6 or Compound 22 having the following structure:

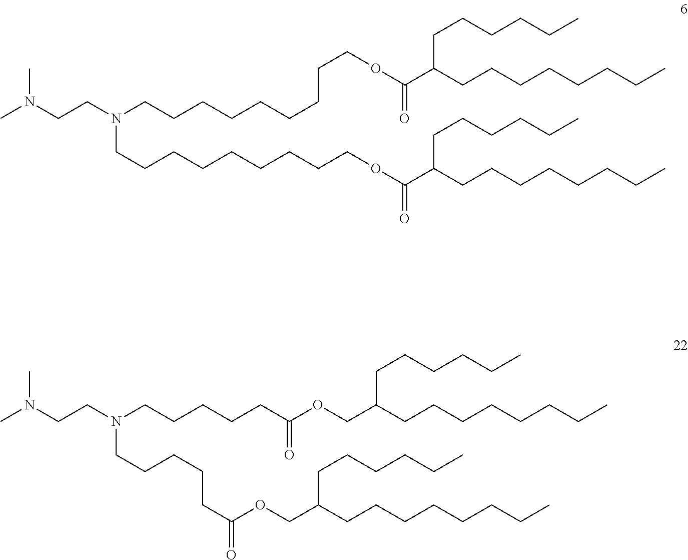

Compounds 6 and 22 are described in WO2015/199952, content of which is incorporated herein by reference in its entirety.

Without limitations, ionizable lipid can comprise 20-90% (mol) of the total lipid present in the lipid nanoparticle. For example, ionizable lipid molar content can be 20-70% (mol), 30-60% (mol) or 40-50% (mol) of the total lipid present in the lipid nanoparticle. In some embodiments, ionizable lipid comprises from about 50 mol % to about 90 mol % of the total lipid present in the lipid nanoparticle.

In some aspects, the lipid nanoparticle can further comprise a non-cationic lipid. Non-ionic lipids include amphipathic lipids, neutral lipids and anionic lipids. Accordingly, the non-cationic lipid can be a neutral uncharged, zwitterionic, or anionic lipid. Non-cationic lipids are typically employed to enhance fusogenicity.

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE. 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, palmitoyl, stearoyl, or oleoyl.

Other examples of non-cationic lipids suitable for use in the lipid nanoparticles include nonphosphorous lipids such as, e.g., stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, sphingomyelin, and the like.

In some embodiments, the non-cationic lipid is a phospholipid. In some embodiments, the non-cationic lipid is selected from DSPC, DPPC, DMPC, DOPC, POPC, DOPE, and SM. In some preferred embodiments, the non-cationic lipid is DPSC.

Exemplary non-cationic lipids are described in PCT Publication WO2017/099823 and US patent publication US2018/0028664, the contents of both of which are incorporated herein by reference in their entirety. In some examples, the non-cationic lipid is oleic acid or a compound of Formula (I),

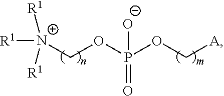

### Formula (II)

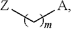

### or Formula (IV),

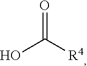

as defined in US2018/0028664, the content of which is incorporated herein by reference in its entirety.

The non-cationic lipid can comprise 0-30% (mol) of the total lipid present in the lipid nanoparticle. For example, the non-cationic lipid content is 5-20% (mol) or 10-15% (mol) of the total lipid present in the lipid nanoparticle. In various embodiments, the molar ratio of ionizable lipid to the neutral lipid ranges from about 2:1 to about 8:1.

In some embodiments, the lipid nanoparticles do not comprise any phospholipids.

In some aspects, the lipid nanoparticle can further comprise a component, such as a sterol, to provide membrane integrity.

One exemplary sterol that can be used in the lipid nanoparticle is cholesterol and derivatives thereof. Non-limiting examples of cholesterol derivatives include polar analogues such as 5α-cholestanol, 5β-coprostanol, cholesteryl-(2′-hydroxy)-ethyl ether, cholesteryl-(4′-hydroxy)-butyl ether, and 6-ketocholestanol: non-polar analogues such as 5α-cholestane, cholestenone, 5α-cholestanone, 5β-cholestanone, and cholesteryl decanoate; and mixtures thereof. In some embodiments, the cholesterol derivative is a polar analogue such as cholesteryl-(4′-hydroxy)-butyl ether.

Exemplary cholesterol derivatives are described in PCT publication WO2009/127060 and US patent publication US2010/0130588, contents of both of which are incorporated herein by reference in their entirety.

The component providing membrane integrity, such as a sterol, can comprise 0-50% (mol) of the total lipid present in the lipid nanoparticle. In some embodiments, such a component is 20-50% (mol) 30-40% (mol) of the total lipid content of the lipid nanoparticle.

In some aspects, the lipid nanoparticle can further comprise a polyethylene glycol (PEG) or a conjugated lipid molecule. Generally, these are used to inhibit aggregation of lipid nanoparticles and/or provide steric stabilization. Exemplary conjugated lipids include, but are not limited to. PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003A077829, US2003/0077829, US2005/0175682, US2008/0020058, US2011/0117125, US2010/0130588, US201610376224, and US2017/0119904, the contents of all of which are incorporated herein by reference in their entirety.

In some embodiments, a PEG-lipid is a compound of Formula (III),

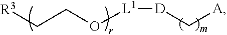

### Formula (III-a-I)

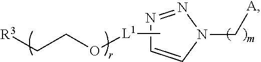

### Formula (III-a-2),

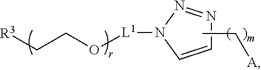

### Formula (III-b-1),

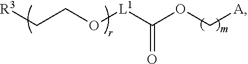

### Formula (III-b-2),

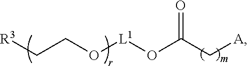

### or Formula (V)

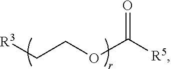

as defined in US2018/0028664, the content of which is incorporated herein by reference in its entirety.

In some embodiments, a PEG-lipid is of Formula (II),

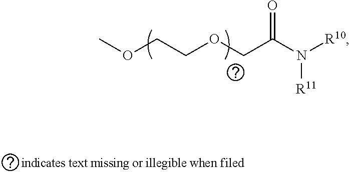

as defined in US20150376115 or in US2016/0376224, the content of both of which is incorporated herein by reference in its entirety.

The PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some examples, the PEG-lipid can be selected from the group consisting of PEG-DMG, 1,2-dimyristoyl-sn-glycero-3-phosphoethanolamine-N-[methoxy(polyethylene glycol)-2000],

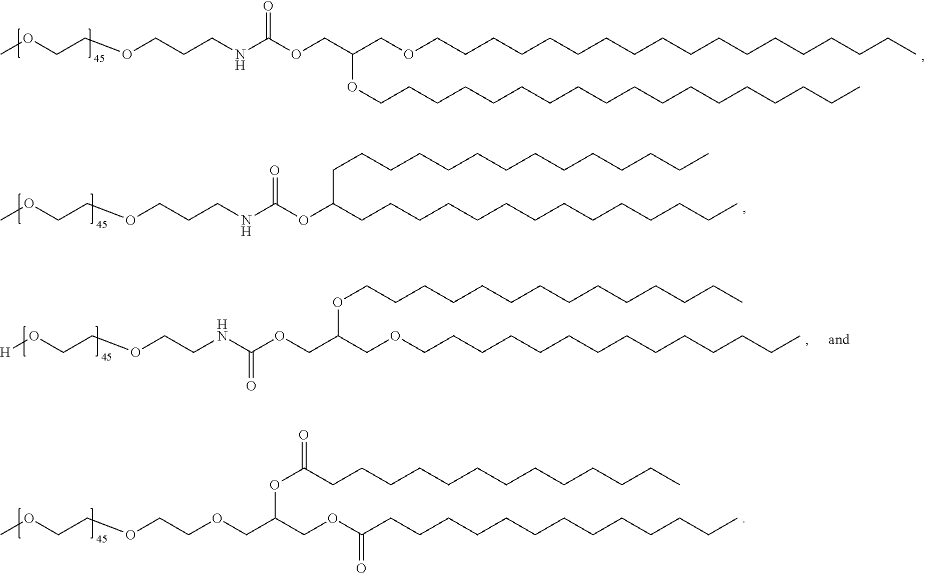

Lipids conjugated with a molecule other than a PEG can also be used in place of PEG-lipid. For example, polyoxazoline (POZ)-lipid conjugates, polyamide-lipid conjugates (such as ATIA-lipid conjugates), and cationic-polymer lipid (CPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT patent application publications WO19961010392, WO1998/051278, WO2002/087541, WO2005/026372, WO2008/147438, WO2009/086558, WO2012/000104, WO2017/117528, WO2017/099823, WO2015/199952, WO2017/004143, WO2015/095346, WO2012/000104, WO2012/000104, and WO2010/006282, US patent application publications US2003/0077829, US2005/0175682, US2008/0020058, US2011/0117125, US2013/0303587, US2018/0028664, US2015/0376115, US2016/0376224, US2016/0317458, US2013/0303587, US2013/0303587, and US20110123453, and U.S. Pat. Nos. 5,885,613, 6,287,591, 6,320,017, and 6,586,559, the contents of all of which are incorporated herein by reference in their entirety.

The PEG or the conjugated lipid can comprise 0-20% (mol) of the total lipid present in the lipid nanoparticle. In some embodiments, PEG or the conjugated lipid content is 0.5-10% or 2-5% (mol) of the total lipid present in the lipid nanoparticle.

Molar ratios of the ionizable lipid, non-cationic-lipid, sterol, and PEG/conjugated lipid can be varied as needed. For example, the lipid particle can comprise 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition comprises 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example comprising 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, I to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation comprises ionizable lipid, phospholipid, cholesterol and a PEG-ylated lipid in a molar ratio of 50:10:38.5:1.5. In some other embodiments, the lipid particle formulation comprises ionizable lipid, cholesterol and a PEG-ylated lipid in a molar ratio of 60:38.5:1.5.

In some embodiments, the lipid particle comprises ionizable lipid, non-cationic lipid (e.g., phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

Lipid nanoparticles (LNPs) comprising ceDNA are disclosed in International Application PCT/US2018/050042, filed on Sep. 7, 2018, which is incorporated herein in its entirety and envisioned for use in the methods and compositions as disclosed herein.

Lipid nanoparticle particle size can be determined by quasi-elastic light scattering using a Malvern Zetasizer Nano ZS (Malvern, UK) and is approximately 50-150 nm diameter, approximately 55-95 nm diameter, or approximately 70-90 nm diameter.

The pKa of formulated cationic lipids can be correlated with the effectiveness of the LNPs for delivery of nucleic acids (see Jayaraman et al, Angewandte Chemie, International Edition (2012), 51(34), 8529-8533; Semple et al, Nature Biotechnology 28, 172-176 (2010), both of which are incorporated by reference in their entirety). The preferred range of pKa is ˜5 to ˜7. The pKa of each cationic lipid is determined in lipid nanoparticles using an assay based on fluorescence of 2-(p-toluidino)-6-napthalene sulfonic acid (TNS). Lipid nanoparticles comprising of cationic lipid/DSPC/cholesterol/PEG-lipid (50/10/38.5/1.5 mol %) in PBS at a concentration of 0.4 mM total lipid can be prepared using the in-line process as described herein and elsewhere. TNS can be prepared as a 100 μM stock solution in distilled water. Vesicles can be diluted to 24 μM lipid in 2 mL of buffered solutions containing, 10 mM HEPES, 10 mM MES, 10 mM ammonium acetate. 130 mM NaCl, where the pH ranges from 2.5 to 11. An aliquot of the TNS solution can be added to give a final concentration of 1 μM and following vortex mixing fluorescence intensity is measured at room temperature in a SLM Aminco Series 2 Luminescence Spectrophotometer using excitation and emission wavelengths of 321 nm and 445 nm. A sigmoidal best fit analysis can be applied to the fluorescence data and the pKa is measured as the pH giving rise to half-maximal fluorescence intensity.

Relative activity can be determined by measuring luciferase expression in the liver 4 hours following administration via tail vein injection. The activity is compared at a dose of 0.3 and 1.0 mg ceDNA/kg and expressed as ng luciferase/g liver measured 4 hours after administration.

Without limitations, a lipid nanoparticle of the invention includes a lipid formulation that can be used to deliver a capsid-free, non-viral DNA vector to a target site of interest (e.g., cell, tissue, organ, and the like). Generally, the lipid nanoparticle comprises capsid-free, non-viral DNA vector and an ionizable lipid or a salt thereof.

In some embodiments, the lipid particle comprises ionizable lipid/non-cationic-lipid/sterol/conjugated lipid at a molar ratio of 50:10:38.5:1.5.

In other aspects, the disclosure provides for a lipid nanoparticle formulation comprising phospholipids, lecithin, phosphatidylcholine and phosphatidylethanolamine.

In some embodiments, one or more additional compounds can also be included. Those compounds can be administered separately or the additional compounds can be included in the lipid nanoparticles of the invention. In other words, the lipid nanoparticles can contain other compounds in addition to the cleavable ceDNA or at least a second ceDNA, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, the one or more additional compound can be a therapeutic agent. The therapeutic agent can be selected from any class suitable for the therapeutic objective. In other words, the therapeutic agent can be selected from any class suitable for the therapeutic objective. In other words, the therapeutic agent can be selected according to the treatment objective and biological action desired. For example, if the cleavable ceDNA within the LNP is useful for treating cancer, the additional compound can be an anti-cancer agent (e.g., a chemotherapeutic agent, a targeted cancer therapy (including, but not limited to, a small molecule, an antibody, or an antibody-drug conjugate). In another example, if the LNP containing the cleavable ceDNA is useful for treating an infection, the additional compound can be an antimicrobial agent (e.g., an antibiotic or antiviral compound). In yet another example, if the LNP containing the cleavable ceDNA is useful for treating an immune disease or disorder, the additional compound can be a compound that modulates an immune response (e.g., an immunosuppressant, immunostimulatory compound, or compound modulating one or more specific immune pathways). In some embodiments, different cocktails of different lipid nanoparticles containing different compounds, such as a cleavable ceDNA encoding a different protein or a different compound, such as a therapeutic may be used in the compositions and methods of the invention.

In some embodiments, the additional compound is an immune modulating agent. For example, the additional compound is an immunosuppressant. In some embodiments, the additional compound is immunostimulatory.

Also provided herein is a pharmaceutical composition comprising the lipid nanoparticle and a pharmaceutically acceptable carrier or excipient.

In some aspects, the disclosure provides for a lipid nanoparticle formulation further comprising one or more pharmaceutical excipients. In some embodiments, the lipid nanoparticle formulation further comprises sucrose, tris, trehalose and/or glycine.

Generally, the lipid nanoparticles of the invention have a mean diameter selected to provide an intended therapeutic effect. Accordingly, in some aspects, the lipid nanoparticle has a mean diameter from about 30 nm to about 150 nm, more typically from about 50 nm to about 150 nm, more typically about 60 nm to about 130 nm, more typically about 70 nm to about 110 nm, most typically about 85 nm to about 105 nm, and preferably about 100 nm. In some aspects, the disclosure provides for lipid particles that are larger in relative size to common nanoparticles and about 150 to 250 nm in size. Lipid nanoparticle particle size can be determined by quasi-elastic light scattering using, for example, a Malvern Zetasizer Nano ZS (Malvern, UK) system.

Depending on the intended use of the lipid particles, the proportions of the components can be varied and the delivery efficiency of a particular formulation can be measured using, for example, an endosomal release parameter (ERP) assay.

The cleavable ceDNA can be complexed with the lipid portion of the particle or encapsulated in the lipid position of the lipid nanoparticle. In some embodiments, the cleavable ceDNA can be fully encapsulated in the lipid position of the lipid nanoparticle, thereby protecting it from degradation by a nuclease, e.g., in an aqueous solution. In some embodiments, the cleavable ceDNA in the lipid nanoparticle is not substantially degraded after exposure of the lipid nanoparticle to a nuclease at 37° C. for at least about 20, 30, 45, or 60 minutes. In some embodiments, the cleavable ceDNA in the lipid nanoparticle is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours.

In certain embodiments, the lipid nanoparticles are substantially non-toxic to a subject, e.g., to a mammal such as a human.

In some aspects, the lipid nanoparticle formulation is a lyophilized powder.

In some embodiments, lipid nanoparticles are solid core particles that possess at least one lipid bilayer. In other embodiments, the lipid nanoparticles have a non-bilayer structure, i.e., a non-lamellar (i.e., non-bilayer) morphology. Without limitations, the non-bilayer morphology can include, for example, three dimensional tubes, rods, cubic symmetries, etc. The non-lamellar morphology (i.e., non-bilayer structure) of the lipid particles can be determined using analytical techniques known to and used by those of skill in the art. Such techniques include, but are not limited to, Cryo-Transmission Electron Microscopy (“Cryo-TEM”), Differential Scanning calorimetry (“DSC”), X-Ray Diffraction, and the like. For example, the morphology of the lipid nanoparticles (lamellar vs. non-lamellar) can readily be assessed and characterized using, e.g., Cryo-TEM analysis as described in US2010/0130588, the content of which is incorporated herein by reference in its entirety.

In some further embodiments, the lipid nanoparticles having a non-lamellar morphology are electron dense.

In some aspects, the disclosure provides for a lipid nanoparticle that is either unilamellar or multilamellar in structure. In some aspects, the disclosure provides for a lipid nanoparticle formulation that comprises multi-vesicular particles and/or foam-based particles.

By controlling the composition and concentration of the lipid components, one can control the rate at which the lipid conjugate exchanges out of the lipid particle and, in turn, the rate at which the lipid nanoparticle becomes fusogenic. In addition, other variables including, e.g., pH, temperature, or ionic strength, can be used to vary and/or control the rate at which the lipid nanoparticle becomes fusogenic. Other methods which can be used to control the rate at which the lipid nanoparticle becomes fusogenic will be apparent to those of ordinary skill in the art based on this disclosure. It will also be apparent that by controlling the composition and concentration of the lipid conjugate, one can control the lipid particle size.

The pKa of formulated cationic lipids can be correlated with the effectiveness of the LNPs for delivery of nucleic acids (see Jayaraman et al, Angewandte Chemie, International Edition (2012), 51(34), 8529-8533; Semple et al, Nature Biotechnology 28, 172-176 (2010), both of which are incorporated by reference in their entirety). The preferred range of pKa is ˜5 to ˜7. The pKa of the cationic lipid can be determined in lipid nanoparticles using an assay based on fluorescence of 2-(p-toluidino)-6-napthalene sulfonic acid (TNS).

Encapsulation of ceDNA in lipid particles can be determined by performing a membrane-impermeable fluorescent dye exclusion assay, which uses a dye that has enhanced fluorescence when associated with nucleic acid, for example, an Oligreen® assay or PicoGreen® assay. Generally, encapsulation is determined by adding the dye to the lipid particle formulation, measuring the resulting fluorescence, and comparing it to the fluorescence observed upon addition of a small amount of nonionic detergent. Detergent-mediated disruption of the lipid bilayer releases the encapsulated ceDNA, allowing it to interact with the membrane-impermeable dye. Encapsulation of ceDNA can be calculated as E=(I₀−I)/I₀, where I and I₀ refers to the fluorescence intensities before and after the addition of detergent.

### IX. Methods of Delivering ceDNA Vectors

In some embodiments, a cleavable ceDNA vector or a cleaved ceDNA can be delivered to a target cell in vitro or in vivo by various suitable methods. ceDNA vectors alone can be applied or injected. CeDNA vectors can be delivered to a cell without the help of a transfection reagent or other physical means. Alternatively, ceDNA vectors can be delivered using any art-known transfection reagent or other art-known physical means that facilitates entry of DNA into a cell, e.g., liposomes, alcohols, polylysine-rich compounds, arginine-rich compounds, calcium phosphate, microvesicles, microinjection, electroporation and the like.

In contrast, transductions with capsid-free AAV vectors disclosed herein can efficiently target cell and tissue-types that are difficult to transduce with conventional AAV virions using various delivery reagent.

In another embodiment, a cleavable ceDNA vector is administered to the CNS (e.g., to the brain or to the eye). The cleavable ceDNA vector may be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes, cortex, basal ganglia, hippocampus and portaamygdala), limbic system, neocortex, corpus striatum, cerebrum, and inferior colliculus. The cleavable ceDNA vector may also be administered to different regions of the eye such as the retina, cornea and/or optic nerve. The cleavable ceDNA vector may be delivered into the cerebrospinal fluid (e.g., by lumbar puncture). The cleavable ceDNA vector may further be administered intravascularly to the CNS in situations in which the blood-brain barrier has been perturbed (e.g., brain tumor or cerebral infarct).

In some embodiments, the cleavable ceDNA vector can be administered to the desired region(s) of the CNS by any route known in the art, including but not limited to, intrathecal, intra-ocular, intracerebral, intraventricular, intravenous (e.g., in the presence of a sugar such as mannitol), intranasal, intra-aural, intra-ocular (e.g., intra-vitreous, sub-retinal, anterior chamber) and peri-ocular (e.g., sub-Tenon's region) delivery as well as intramuscular delivery with retrograde delivery to motor neurons.

Tn some embodiments, the cleavable ceDNA vector is administered in a liquid formulation by direct injection (e.g., stereotactic injection) to the desired region or compartment in the CNS. In other embodiments, the cleavable ceDNA vector can be provided by topical application to the desired region or by intra-nasal administration of an aerosol formulation. Administration to the eye may be by topical application of liquid droplets. As a further alternative, the cleavable ceDNA vector can be administered as a solid, slow-release formulation (see, e.g., U.S. Pat. No. 7,201,898). In yet additional embodiments, the cleavable ceDNA vector can used for retrograde transport to treat, ameliorate, and/or prevent diseases and disorders involving motor neurons (e.g., amyotrophic lateral sclerosis (ALS); spinal muscular atrophy (SMA), etc.). For example, the cleavable ceDNA vector can be delivered to muscle tissue from which it can migrate into neurons.

### X. Additional Uses of the ceDNA Vectors

The compositions and cleavable ceDNA vectors provided herein can be used in combination with, e.g., a nuclease and at least one gRNA, to gene edit a target gene for various purposes. In some embodiments, the resulting transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. In some embodiments, the resulting transgene encodes one or more peptides, polypeptides, or proteins, which are useful for the treatment, prevention, or amelioration of disease states or disorders in a mammalian subject. The resulting transgene can be transferred (e.g., expressed in) to a subject in a sufficient amount to treat a disease associated with reduced expression, lack of expression or dysfunction of the gene. In some embodiments the resulting transgene can be expressed in a subject in a sufficient amount to treat a disease associated with increased expression, activity of the gene product, or inappropriate upregulation of a gene that the resulting transgene suppresses or otherwise causes the expression of which to be reduced. In yet other embodiments, the resulting transgene replaces or supplements a defective copy of the native gene. It will be appreciated by one of ordinary skill in the art that the transgene may not be an open reading frame of a gene to be transcribed itself; instead it may be a promoter region or repressor region of a target gene, and the cleavable ceDNA vector may modify such region with the outcome of so modulating the expression of a gene of interest.

In some embodiments, the transgene encodes a protein or functional RNA that is intended to be used to create an animal model of disease. In some embodiments, the transgene encodes one or more peptides, polypeptides, or proteins, which are useful for the treatment or prevention of disease states in a mammalian subject. The transgene or donor sequence can be transferred (e.g., expressed in) to a patient in a sufficient amount to treat a disease associated with reduced expression, lack of expression or dysfunction of the gene.

### XL Methods of Use

The cleavable ceDNA vector as disclosed herein can also be used in a method for the delivery of a nucleotide sequence of interest (e.g., a transgene cassette) to a target cell (e.g., a host cell). The method may in particular be a method for delivering a cleaved ceDNA, nuclease enzyme, and at lest one guide RNA to a cell of a subject in need thereof and for editing a target gene of interest. The invention allows for the in vivo expression of gene editing molecules, e.g., a nuclease and/or a guide sequence in a cell in a subject such that therapeutic effect of the gene editing machinery occurs. These results are seen with both in vivo and in vitro modes of ceDNA vector delivery.

In addition, the invention provides a method for the delivery of a gene editing system to a cell of a subject in need thereof, comprising multiple administrations of the cleavable ceDNA vector comprising a transgene or transgene cassette of interest. Since the cleavable ceDNA vector does not induce an immune response like that typically observed against encapsidated viral vectors, such a multiple administration strategy will likely have greater success in a cleavable ceDNA-based system.

The cleavable ceDNA vector nucleic acid(s) are administered in sufficient amounts to transfect the cells of a desired tissue and to provide sufficient levels of gene transfer and expression without undue adverse effects. Conventional and pharmaceutically acceptable routes of administration include, but are not limited to, intravenous (e.g., in a liposome formulation), direct delivery to the selected organ (e.g., intraportal delivery to the liver), intramuscular, and other parental routes of administration. Routes of administration may be combined, if desired.

ceDNA delivery is not limited to vector delivery of all nucleotides encoding gene editing components. For example, ceDNA vectors as described herein may be used with other delivery systems provided to provide a portion of the gene editing components. One non-limiting example of a system that may be combined with ceDNA vectors in accordance with the present disclosure includes systems which separately deliver Cas9 and/or a gRNA to a host cell in need of treatment or gene editing. In certain embodiments, Cas9 may be delivered in a nanoparticle such as those described in Lee et al., Nanoparticle delivery of Cas9 ribonucleotideprotein and donor DNA in vivo induces homology-directed DNA repair, Nature Biomedical Engineering, 2017 (herein incorporated by reference in its entirety), while other components, such as a donor sequence are provided by ceDNA.

The invention also provides for a method of treating a disease in a subject comprising introducing into a target cell in need thereof (in particular a muscle cell or tissue) of the subject a therapeutically effective amount of a cleavable ceDNA vector, optionally with a pharmaceutically acceptable carrier. While the cleavable ceDNA vector can be introduced in the presence of a carrier, such a carrier is not required. The cleavable ceDNA vector implemented comprises a nucleotide sequence of interest useful for treating the disease. In particular, the cleavable ceDNA vector may comprise a desired exogenous DNA sequence operably linked to control elements capable of directing transcription of the desired polypeptide, protein, or oligonucleotide encoded by the exogenous DNA sequence when introduced into the subject. The cleavable ceDNA vector can be administered via any suitable route as provided above, and elsewhere herein.

The compositions and vectors provided herein can be used to deliver a transgene for various purposes. In some embodiments, the transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. In some embodiments, the transgene encodes one or more peptides, polypeptides, or proteins, which are useful for the treatment or prevention of disease states in a mammalian subject. The transgene can be transferred (e.g., expressed in) to a patient in a sufficient amount to treat a disease associated with reduced expression, lack of expression or dysfunction of the gene.

In principle, the expression cassette can include a nucleic acid or nuclease targeting any gene that encodes a protein or polypeptide that is either reduced or absent due to a mutation or which conveys a therapeutic benefit when overexpressed is considered to be within the scope of the invention. The cleavable ceDNA vector comprises a template nucleotide sequence used as a correcting DNA strand to be inserted after a double-strand break provided by a meganuclease- or zinc finger nuclease. The cleavable ceDNA vector can comprise a template nucleotide sequence used as a correcting DNA strand to be inserted after a double-strand break provided by a meganuclease- or zinc finger nuclease. Preferably, noninserted bacterial DNA is not present and preferably no bacterial DNA is present in the cleavable ceDNA compositions provided herein.

A cleavable ceDNA is not limited to one species of ceDNA vector. As such, in another aspect, multiple ceDNA vectors comprising different transgene sequences can be delivered simultaneously or sequentially to the target cell, tissue, organ, or subject. Therefore, this strategy can allow for the gene-editing of multiple genes simultaneously. It is also possible to separate different portions of the gene editing functionality into separate vectors which can be administered simultaneously or at different times, and can be separately regulatable. Delivery can also be performed multiple times and, importantly for gene therapy in the clinical setting, in subsequent increasing or decreasing doses, given the lack of an anti-capsid host immune response due to the absence of a viral capsid. It is anticipated that no anti-capsid response will occur as there is no capsid.

The invention also provides for a method of treating a disease in a subject comprising introducing into a target cell in need thereof (in particular a muscle cell or tissue) of the subject a therapeutically effective amount of a cleaved ceDNA vector with a nuclease and at least one guide RNA, optionally with a pharmaceutically acceptable carrier. While the cleavable ceDNA vector can be introduced in the presence of a carrier, such a carrier is not required. The cleavable ceDNA vector implemented comprises a nucleotide sequence of interest useful for treating the disease. In particular, the cleavable ceDNA vector may comprise a desired exogenous DNA sequence operably linked to control elements capable of directing transcription of the desired polypeptide, protein, or oligonucleotide encoded by the exogenous DNA sequence when introduced into the subject. The cleavable ceDNA vector can be administered via any suitable route as provided above, and elsewhere herein.

### XII. Methods of Treatment

The technology described herein also demonstrates methods for making, as well as methods of using the disclosed ceDNA vectors in a variety of ways, including, for example, ex situ, in vitro and in vivo applications, methodologies, diagnostic procedures, and/or gene therapy regimens.

Provided herein is a method of treating a disease or disorder in a subject comprising introducing into a target cell in need thereof (for example, a muscle cell or tissue, or other affected cell type) of the subject a therapeutically effective amount of a ceDNA vector alone or in combination with a nuclease and at least one gRNA, optionally with a pharmaceutically acceptable carrier. While the cleavable ceDNA vector can be introduced in the presence of a carrier, such a carrier is not required. The cleavable ceDNA vector implemented comprises a nucleotide sequence of interest useful for treating the disease. In particular, the cleavable ceDNA vector may comprise a desired exogenous DNA sequence operably linked to control elements capable of directing transcription of the desired polypeptide, protein, or oligonucleotide encoded by the exogenous DNA sequence when introduced into the subject. The cleavable ceDNA vector can be administered via any suitable mute as provided above, and elsewhere herein.

Disclosed herein are ceDNA vector compositions and formulations that include one or more of the cleavable ceDNA vectors of the present invention together with one or more pharmaceutically-acceptable buffers, diluents, or excipients. Such compositions may be included in one or more diagnostic or therapeutic kits, for diagnosing, preventing, treating or ameliorating one or more symptoms of a disease, injury, disorder, trauma or dysfunction. In one aspect the disease, injury, disorder, trauma or dysfunction is a human disease, injury, disorder, trauma or dysfunction.

Another aspect of the technology described herein provides a method for providing a subject in need thereof with a diagnostically- or therapeutically-effective amount of a cleavable ceDNA vector, the method comprising providing to a cell, tissue or organ of a subject in need thereof, an amount of the cleavable ceDNA vector as disclosed herein; and for a time effective to enable expression of the transgene from the cleavable ceDNA vector thereby providing the subject with a diagnostically- or a therapeutically-effective amount of the protein, peptide, nucleic acid expressed by the cleavable ceDNA vector. In a further aspect, the subject is human.

Another aspect of the technology described herein provides a method for diagnosing, preventing, treating, or ameliorating at least one or more symptoms of a disease, a disorder, a dysfunction, an injury, an abnormal condition, or trauma in a subject. In an overall and general sense, the method includes at least the step of administering to a subject in need thereof one or more of the disclosed ceDNA vectors, in an amount and for a time sufficient to diagnose, prevent, treat or ameliorate the one or more symptoms of the disease, disorder, dysfunction, injury, abnormal condition, or trauma in the subject. In a further aspect, the subject is human.

Another aspect is use of the cleavable ceDNA vector as a tool for treating or reducing one or more symptoms of a disease or disease states. There are a number of inherited diseases in which defective genes are known, and typically fall into two classes: deficiency states, usually of enzymes, which are generally inherited in a recessive manner, and unbalanced states, which may involve regulatory or structural proteins, and which are typically but not always inherited in a dominant manner. For deficiency state diseases, ceDNA vectors can be used to deliver transgenes to bring a normal gene into affected tissues for replacement therapy, as well, in some embodiments, to create animal models for the disease using antisense mutations. For unbalanced disease states, ceDNA vectors can be used to create a disease state in a model system, which could then be used in efforts to counteract the disease state. Thus the cleavable ceDNA vectors and methods disclosed herein permit the treatment of genetic diseases. As used herein, a disease state is treated by partially or wholly remedying the deficiency or imbalance that causes the disease or makes it more severe.

### A. Host Cells

In some embodiments, the cleavable ceDNA vector delivers the transgene into a subject host cell. In some embodiments, the subject host cell is a human host cell, including, for example blood cells, stem cells, hematopoietic cells, CD34⁺ cells, liver cells, cancer cells, vascular cells, muscle cells, pancreatic cells, neural cells, ocular or retinal cells, epithelial or endothelial cells, dendritic cells, fibroblasts, or any other cell of mammalian origin, including, without limitation, hepatic (i.e., liver) cells, lung cells, cardiac cells, pancreatic cells, intestinal cells, diaphragmatic cells, renal (i.e., kidney) cells, neural cells, blood cells, bone marrow cells, or any one or more selected tissues of a subject for which gene therapy is contemplated. In one aspect, the subject host cell is a human host cell.

The present disclosure also relates to recombinant host cells as mentioned above, including ceDNA vectors as described herein. Thus, one can use multiple host cells depending on the purpose as is obvious to the skilled artisan. A construct or ceDNA vector including donor sequence is introduced into a host cell so that the donor sequence is maintained as a chromosomal integrant as described earlier. The term host cell encompasses any progeny of a parent cell that is not identical to the parent cell due to mutations that occur during replication. The choice of a host cell will to a large extent depend upon the donor sequence and its source. The host cell may also be a eukaryote, such as a mammalian, insect, plant, or fungal cell. In one embodiment, the host cell is a human cell (e.g., a primary cell, a stem cell, or an immortalized cell line). In some embodiments, the host cell is gene edited for correction of a defective gene or to ablate expression of a gene. For Example, CRISPR/CAS can be used to edit the genome with one or more gRNA by either NHEJ, MMEJ, or HDR repair, as well as other gene editing systems, e.g., ZFN or TALENs. The host cell can be any cell type, e.g., a somatic cell or a stem cell, an induced pluripotent stem cell, or a blood cell, e.g., T-cell or B-cell, or bone marrow cell. In certain embodiments, the host cell is an allogenic cell. For example, T-cell genome engineering is useful for cancer immunotherapies, disease modulation such as HIV therapy (e.g., receptor knock out, such as CXCR4 and CCR5) and immunodeficiency therapies. MHC receptors on B-cells can be targeted for immunotherapy. Genome edited bone marrow stem cells, e.g., CD34⁺ cells, or induced pluripotent stem cells can be transplanted back into a patient for expression of a therapeutic protein.

### B. Exemplary Diseases to be Treated

The cleavable ceDNA vectors are also useful for ablating gene expression. For example, in one embodiment a cleavable ceDNA vector can be used to cause a nonsense indel (e.g., an insertion or deletion of non-coding base pairs) to induce knockdown of a target gene, for example, by causing a frame-shift mutation. As a non-limiting example, expression of CXCR4 and CCR5, HIV receptors, have been successfully ablated in primary human T-cells by induction of either NHEJ. MMEJ, or HDR pathways using CAS9 RNP and one or mom guide RNA, See Schumann et al. (2015) Generation of knock in primary human cells using Cas9 ribonucleoproteins. PNAS 112(33): 10437-10442, herein incorporated by reference in its entirety. This system required only a single guide RNA and RNP (e.g., CAS9). CeDNA vectors can also be used to target the PD-1 locus in order to ablate expression. PD-1 expresses an immune checkpoint cell surface receptor on chronically active T cells that happens in malignancy. See Schumann et al. supra.

In some embodiments, the cleavable ceDNA vectors are used for correcting a defective gene by using a vector that targets the diseased gene. In one embodiment, the cleavable ceDNA vectors as described herein can be used to excise a desired region of DNA to correct a frameshift mutation, for example, to treat Duchenne muscular dystrophy or to remove mutated introns of LCA10 in the treatment of Leber Congenital Amaurosis. Non-limiting examples of diseases or disorders amenable to treatment by gene editing using ceDNA vectors in combination with a nuclease and at least one guide RNA, are listed in Tables A-C along with their and their associated genes of US patent publication 2014/0170753, which is herein incorporated by reference in its entirety. In alternative embodiments, the cleavable ceDNA vectors are used for insertion of an expression cassette for expression of a therapeutic protein or reporter protein in a safe harbor gene, e.g., in an inactive intron. In certain embodiments, a promoter-less cassette is inserted into the safe harbor gene. In such embodiments, a promoter-less cassette can take advantage of the safe harbor gene regulatory elements (promoters, enhancers, and signaling peptides), a non-limiting example of insertion at the safe harbor locus is insertion into to the albumin locus that is described in Blood (2015) 126 (15): 1777-1784, which is incorporated herein by reference in its entirety. Insertion into Albumin has the benefit of enabling secretion of the transgene into the blood (See e.g., Example 22). In addition, a genomic safe harbor site can be determined using techniques known in the art and described in, for example, Papapetrou, E R & Schambach, A. Molecular Therapy 24(4):678-684 (2016) or Sadelain et al. Nature Reviews Cancer 12:51-58 (2012), the contents of each of which are incorporated herein by reference in their entirety. It is specifically contemplated herein that safe harbor sites in an adeno associated virus (AAV) genome (e.g., AAVS1 safe harbor site) can be used with the methods and compositions described herein (see e.g., Oceguera-Yanez et al. Methods 101:43-55 (2016) or Tiyaboonchai, A et al. Stem Cell Res 12(3):630-7 (2014), the contents of each of which are incorporated by reference in their entirety). For example, the AAVS1 genomic safe harbor site can be used with the cleavable ceDNA vectors and compositions as described herein for the purposes of hematopoietic specific transgene expression and gene silencing in embryonic stem cells (e.g., human embryonic stem cells) or induced pluripotent stem cells (iPS cells). In addition, it is contemplated herein that synthetic or commercially available HDR repair templates for insertion into an AASV1 safe harbor site on chromosome 19 can be used with the cleavable ceDNA vectors or compositions as described herein. For example, homology-directed recombination repair templates, and guide RNA, can be purchased commercially, for example, from System Biosciences, Palo Alto, CA, and cloned into a cleavable ceDNA vector.

In some embodiments, the cleavable ceDNA vectors are used for knocking out or editing a gene in a T cell, e.g., to engineer the T cell for improved adoptive cell transfer and/or CAR-T therapies (see, e.g., Example 24). In some embodiments, the cleavable ceDNA vector can comprise a template nucleic acid sequence. Non-limiting examples of therapeutically relevant knock-outs and gene editing of T cells are described in PNAS (2015) 112(33):10437-10442, which is incorporated herein by reference in its entirety.

The eDNA vector or a composition thereof can be used in the treatment of any hereditary disease. As a non-limiting example, the cleavable ceDNA vector or a composition thereof e.g., can be used in the treatment of transthyretin amyloidosis (ATTR), an orphan disease where the mutant protein misfolds and aggregates in nerves, the heart, the gastrointestinal system etc. It is contemplated herein that the disease can be treated by deletion of the mutant disease gene (mutTTR) using the gene editing systems described herein. Such treatments of hereditary diseases can halt disease progression and may enable regression of an established disease or reduction of at least one symptom of the disease by at least 10%.

In another embodiment, the cleavable ceDNA vector or a composition thereof can be used in the treatment of omithine transcarbamylase deficiency (OTC deficiency), hyperammonemia or other urea cycle disorders, which impair a neonate or infant's ability to detoxify ammonia. As with all diseases of inborn metabolism, it is contemplated herein that even a partial restoration of enzyme activity compared to wild-type controls (e.g., at least 20%, at least 30%, at least 40%, at least 50%, at least 60%, at least 70%, at least 80%, at least 90%, at least 95% or at least 99%) may be sufficient for reduction in at least one symptom OTC and/or an improvement in the quality of life for a subject having OTC deficiency. In one embodiment, a nucleic acid encoding OTC can be inserted behind the albumin endogenous promoter for in vivo protein replacement.

In another embodiment, the cleavable ceDNA vector or a composition thereof can be used in the treatment of phenylketonuria (PKU) by delivering a nucleic acid sequence encoding a phenylalanine hydroxylase enzyme to reduce buildup of dietary phenylalanine, which can be toxic to PKU sufferers. As with all diseases of inborn metabolism, it is contemplated herein that even a partial restoration of enzyme activity compared to wild-type controls (e.g., at least 20%, at least 30%, at least 40%, at least 50%, at least 60%, at least 70%, at least 80%, at least 90%, at least 95% or at least 99%) may be sufficient for reduction in at least one symptom of PKU and/or an improvement in the quality of life for a subject having PKU. In one embodiment, a nucleic acid encoding phenylalanine hydroxylase can be inserted behind the albumin endogenous promoter for in vivo protein replacement.

In another embodiment, the cleavable ceDNA vector or a composition thereof can be used in the treatment of glycogen storage disease (GSD) by delivering a nucleic acid sequence encoding an enzyme to correct aberrant glycogen synthesis or breakdown in subjects having GSD. Non-limiting examples of enzymes that can be corrected using the gene editing methods described herein include glycogen synthase, glucose-6-phosphatase, acid-alpha glucosidase, glycogen debranching enzyme, glycogen branching enzyme, muscle glycogen phosphorylase, liver glycogen phosphorylase, muscle phosphofructokinase, phosphorylase kinase, glucose transporter-2 (GLUT-2), aldolase A, beta-enolase, phosphoglucomutase-1 (PGM-1), and glycogenin-1. As with all diseases of inborn metabolism, it is contemplated herein that even a partial restoration of enzyme activity compared to wild-type controls (e.g., at least 20%, at least 30%, at least 40%, at least 50%, at least 60%, at least 70%, at least 80%, at least 90%, at least 95% or at least 99%) may be sufficient for reduction in at least one symptom of GSD and/or an improvement in the quality of life for a subject having GSD. In one embodiment, a nucleic acid encoding an enzyme to correct aberrant glycogen storage can be inserted behind the albumin endogenous promoter for in vivo protein replacement.

The cleavable ceDNA vectors described herein are also contemplated for use in the in vivo repair of Leber congenital amaurosis (LCA), polyglutamine diseases, including polyQ repeats, and alpha-1 antitrypsin deficiency (A1AT). LCA is a rare congenital eye disease resulting in blindness, which can be caused by a mutation in any one of the following genes: GUCY2D, RPE65, SPATA7, AIPL1, LCA5, RPGRIP1, CRX, CRB1, NMNAT1, CEP290, IMPDH1, RD3, RDH12, LRAT, TULP1, KCNJ13, GDF6 and/or PRPH2. It is contemplated herein that the gene editing methods and compositions as described herein can be adapted for delivery of one or more of the genes associated with LCA in order to correct an error in the gene(s) responsible for the symptoms of LCA. Polyglutamine diseases include, but are not limited to: dentatorubropallidoluysian atrophy, Huntington's disease, spinal and bulbar muscular atrophy, and spinocerebellar ataxia types 1, 2, 3 (also known as Machado-Joseph disease), 6, 7, and 17. It is specifically contemplated herein that the gene editing methods can be used to repair DNA mutations resulting in trinucleotide repeat expansions (e.g., polyQ repeats), such as those associated with polyglutamine diseases. A1AT deficiency is a genetic disorder that causes defective production of alpha-1 antitrypsin, leading to decreased activity of the enzyme in the blood and lungs, which in turn can lead to emphysema or chronic obstructive pulmonary disease in affected subjects. Repair of A1AT deficiency is specifically contemplated herein using the cleavable ceDNA vectors or compositions thereof as outlined herein. It is contemplated herein that a nucleic acid encoding a desired protein for the treatment of LCA, polyglutamine diseases or A1AT deficiency can be inserted behind the albumin endogenous promoter for in vivo protein replacement.

In further embodiments, the compositions comprising a cleavable ceDNA vector as described herein can be used to edit a gene in a viral sequence, a pathogen sequence, a chromosomal sequence, a translocation junction (e.g., a translocation associated with cancer), a non-coding RNA gene or RNA sequence, a disease associated gene, among others.

Any nucleic acid or target gene of interest may be edited using the ceDNA vector and, e.g., a nuclease and at least one gRNA as disclosed herein. Target nucleic acids and target genes include, but are not limited to nucleic acids encoding polypeptides, or non-coding nucleic acids (e.g., RNAi, miRs etc.) preferably therapeutic (e.g., for medical, diagnostic, or veterinary uses) or immunogenic (e.g., for vaccines) polypeptides. In certain embodiments, the target nucleic acids or target genes that are targeted by the ceDNA vectors as described herein encode one or more polypeptides, peptides, ribozymes, peptide nucleic acids, siRNAs, RNAis, antisense oligonucleotides, antisense polynucleotides, antibodies, antigen binding fragments, or any combination thereof.

In particular, a gene target for gene editing can encode, for example, but is not limited to, protein(s), polypeptide(s), peptide(s), enzyme(s), antibodies, antigen binding fragments, as well as variants, and/or active fragments thereof, for use in the treatment, prophylaxis, and/or amelioration of one or more symptoms of a disease, dysfunction, injury, and/or disorder. In one aspect, the disease, dysfunction, trauma, injury and/or disorder is a human disease, dysfunction, trauma, injury, and/or disorder.

As noted herein, the gene target for gene editing can encode a protein or peptide, or therapeutic nucleic acid sequence or therapeutic agent, including but not limited to one or more agonists, antagonists, anti-apoptosis factors, inhibitors, receptors, cytokines, cytotoxins, erythropoietic agents, glycoproteins, growth factors, growth factor receptors, hormones, hormone receptors, interferons, interleukins, interleukin receptors, nerve growth factors, neuroactive peptides, neuroactive peptide receptors, proteases, protease inhibitors, protein decarboxylases, protein kinases, protein kinase inhibitors, enzymes, receptor binding proteins, transport proteins or one or more inhibitors thereof, serotonin receptors, or one or more uptake inhibitors thereof, serpins, serpin receptors, tumor suppressors, diagnostic molecules, chemotherapeutic agents, cytotoxins, or any combination thereof.

### C. Additional Diseases

In general, the cleavable ceDNA vector as disclosed herein can be used to deliver any transgene in accordance with the description above to treat, prevent, or ameliorate the symptoms associated with any disorder related to gene expression. Illustrative disease states include, but are not-limited to: cystic fibrosis (and other diseases of the lung), hemophilia A, hemophilia B, thalassemia, anemia and other blood disorders, AIDS, Alzheimer's disease, Parkinson's disease, Huntington's disease, amyotrophic lateral sclerosis, epilepsy, and other neurological disorders, cancer, diabetes mellitus, muscular dystrophies (e.g., Duchenne, Becker), Hurler's disease, adenosine deaminase deficiency, metabolic defects, retinal degenerative diseases (and other diseases of the eye), mitochondriopathies (e.g., Leber's hereditary optic neuropathy (LHON), Leigh syndrome, and subacute sclerosing encephalopathy), myopathies (e.g., facioscapulohumeral myopathy (FSHD) and cardiomyopathies), diseases of solid organs (e.g., brain, liver, kidney, heart), and the like. In some embodiments, the cleavable ceDNA vectors as disclosed herein can be advantageously used in the treatment of individuals with metabolic disorders (e.g., ornithine transcarbamylase deficiency).

In some embodiments, the cleavable ceDNA vector described herein can be used to treat, ameliorate, and/or prevent a disease or disorder caused by mutation in a gene or gene product. Exemplary diseases or disorders that can be treated with a cleavable ceDNA vectors include, but are not limited to, metabolic diseases or disorders (e.g., Fabry disease, Gaucher disease, phenylketonuria (PKU), glycogen storage disease); urea cycle diseases or disorders (e.g., ornithine transcarbamylase (OTC) deficiency); lysosomal storage diseases or disorders (e.g., metachromatic leukodystrophy (MLD), mucopolysaccharidosis Type II (MPSII; Hunter syndrome)); liver diseases or disorders (e.g., progressive familial intrahepatic cholestasis (PFIC); blood diseases or disorders (e.g., hemophilia (A and B), thalassemia, and anemia); cancers and tumors, and genetic diseases or disorders (e.g., cystic fibrosis).

As still a further aspect, a cleavable ceDNA vector as disclosed herein may be employed to deliver a heterologous nucleotide sequence in situations in which it is desirable to regulate the level of transgene expression (e.g., transgenes encoding hormones or growth factors, as described herein).

Accordingly, in some embodiments, the cleavable ceDNA vector described herein can be used to correct an abnormal level and/or function of a gene product (e.g., an absence of, or a defect in, a protein) that results in the disease or disorder. The cleavable ceDNA vector can produce a functional protein and/or modify levels of the protein to alleviate or reduce symptoms resulting from, or confer benefit to, a particular disease or disorder caused by the absence or a defect in the protein. For example, treatment of OTC deficiency can be achieved by producing functional OTC enzyme; treatment of hemophilia A and B can be achieved by modifying levels of Factor VIII, Factor IX, and Factor X; treatment of PKU can be achieved by modifying levels of phenylalanine hydroxylase enzyme; treatment of Fabry or Gaucher disease can be achieved by producing functional alpha galactosidase or beta glucocerebrosidase, respectively; treatment of MILD or MPSII can be achieved by producing functional arylsulfatase A or iduronate-2-sulfatase, respectively; treatment of cystic fibrosis can be achieved by producing functional cystic fibrosis transmembrane conductance regulator; treatment of glycogen storage disease can be achieved by restoring functional G6Pase enzyme function; and treatment of PFIC can be achieved by producing functional ATP8B1, ABCB11, ABCB4, or TJP2 genes.

In alternative embodiments, the cleavable ceDNA vectors as disclosed herein can be used to provide an antisense nucleic acid to a cell in vitro or in vivo. For example, where the transgene is a RNAi molecule, expression of the antisense nucleic acid or RNAi in the target cell diminishes expression of a particular protein by the cell. Accordingly, transgenes which are RNAi molecules or antisense nucleic acids may be administered to decrease expression of a particular protein in a subject in need thereof. Antisense nucleic acids may also be administered to cells in vitro to regulate cell physiology, e.g., to optimize cell or tissue culture systems.

In some embodiments, exemplary transgenes encoded by the cleavable ceDNA vector include, but are not limited to: X, lysosomal enzymes (e.g., hexosaminidase A, associated with Tay-Sachs disease, or iduronate sulfatase, associated, with Hunter Syndrome/MPS II), erythropoietin, angiostatin, endostatin, superoxide dismutase, globin, leptin, catalase, tyrosine hydroxylase, as well as cytokines (e.g., a interferon, β-interferon, interferon-γ, interleukin-2, interleukin-4, interleukin 12, granulocyte-macrophage colony stimulating factor, lymphotoxin, and the like), peptide growth factors and hormones (e.g., somatotropin, insulin, insulin-like growth factors 1 and 2, platelet derived growth factor (PDGF), epidermal growth factor (EGF), fibroblast growth factor (FGF), nerve growth factor (NGF), neurotrophic factor-3 and 4, brain-derived neurotrophic factor (BDNF), glial derived growth factor (GDNF), transforming growth factor-α and -β, and the like), receptors (e.g., tumor necrosis factor receptor). In some exemplary embodiments, the transgene encodes a monoclonal antibody specific for one or more desired targets. In some exemplary embodiments, more than one transgene is encoded by the cleavable ceDNA vector. In some exemplary embodiments, the transgene encodes a fusion protein comprising two different polypeptides of interest. In some embodiments, the transgene encodes an antibody, including a full-length antibody or antibody fragment, as defined herein. In some embodiments, the antibody is an antigen-binding domain or an immunoglobulin variable domain sequence, as that is defined herein. Other illustrative transgene sequences encode suicide gene products (thymdine kinase, cytosine deaminase, diphtheria toxin, cytochrome P450, deoxycytidine kinase, and tumor necrosis factor), proteins conferring resistance to a drug used in cancer therapy, and tumor suppressor gene products.

In a representative embodiment, the transgene expressed by the cleavable ceDNA vector can be used for the treatment of muscular dystrophy in a subject in need thereof, the method comprising: administering a treatment-, amelioration- or prevention-effective amount of ceDNA vector described herein, wherein the cleavable ceDNA vector comprises a heterologous nucleic acid encoding dystrophin, a mini-dystrophin, a micro-dystrophin, myostatin propeptide, follistatin, activin type II soluble receptor, IGF-1, anti-inflammatory polypeptides such as the Ikappa B dominant mutant, sarcospan, utrophin, a micro-dystrophin, laminin-α2, α-sarcoglycan, β-sarcoglycan, γ-sarcoglycan, δ-sarcoglycan, IGF-1, an antibody or antibody fragment against myostatin or myostatin propeptide, and/or RNAi against myostatin. In particular embodiments, the cleavable ceDNA vector can be administered to skeletal, diaphragm and/or cardiac muscle as described elsewhere herein.

In some embodiments, the cleavable ceDNA vector can be used to deliver a transgene to skeletal, cardiac or diaphragm muscle, for production of a polypeptide (e.g., an enzyme) or functional RNA (e.g., RNAi, microRNA, antisense RNA) that normally circulates in the blood or for systemic delivery to other tissues to treat, ameliorate, and/or prevent a disorder (e.g., a metabolic disorder, such as diabetes (e.g., insulin), hemophilia (e.g., VIII), a mucopolysaccharide disorder (e.g., Sly syndrome, Hurler Syndrome, Scheie Syndrome, Hurler-Scheie Syndrome. Hunter's Syndrome. Sanfilippo Syndrome A, B. C. D, Morquio Syndrome, Maroteaux-Lamy Syndrome, etc.) or a lysosomal storage disorder (such as Gaucher's disease [glucocerebrosidase], Pompe disease [lysosomal acid .alpha.-glucosidase] or Fabry disease [.alpha.-galactosidase A]) or a glycogen storage disorder (such as Pompe disease [lysosomal acid a glucosidase]). Other suitable proteins for treating, ameliorating, and/or preventing metabolic disorders are described above.

In other embodiments, the cleavable ceDNA vector as disclosed herein can be used to deliver a transgene in a method of treating, ameliorating, and/or preventing a metabolic disorder in a subject in need thereof. Illustrative metabolic disorders and transgenes encoding polypeptides are described herein. Optionally, the polypeptide is secreted (e.g., a polypeptide that is a secreted polypeptide in its native state or that has been engineered to be secreted, for example, by operable association with a secretory signal sequence as is known in the art).

Another aspect of the invention relates to a method of treating, ameliorating, and/or preventing congenital heart failure or PAD in a subject in need thereof, the method comprising administering a cleavable ceDNA vector as described herein to a mammalian subject, wherein the cleavable ceDNA vector comprises a transgene encoding, for example, a sarcoplasmic endoreticulum Ca²⁺-ATPase (SERCA2a), an angiogenic factor, phosphatase inhibitor 1(1-1), RNAi against phospholamban: a phospholamban inhibitory or dominant-negative molecule such as phospholamban S16E, a zinc finger protein that regulates the phospholamban gene, β2-adrenergic receptor. β2-adrenergic receptor kinase (BARK), PI3 kinase, calsarcan, a β-adrenergic receptor kinase inhibitor (βARKct), inhibitor 1 of protein phosphatase 1, S100A1, parvalbumin, adenylyl cyclase type 6, a molecule that effects G-protein coupled receptor kinase type 2 knockdown such as a truncated constitutively active βARKct, Pim-1, PGC-1α, SOD-1, SOD-2, EC-SOD, kallikrein, HIF, thymosin-β4, mir-1, mir-133, mir-206 and/or mir-208.

The cleavable ceDNA vectors as disclosed herein can be administered to the lungs of a subject by any suitable means, optionally by administering an aerosol suspension of respirable particles comprising the cleavable ceDNA vectors, which the subject inhales. The respirable particles can be liquid or solid. Aerosols of liquid particles comprising the cleavable ceDNA vectors may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the cleavable ceDNA vectors may likewise be produced with any solid particulate medicament aerosol generator, by techniques known in the pharmaceutical art.

In some embodiments, the cleavable ceDNA vectors can be administered to tissues of the CNS (e.g., brain, eye). In particular embodiments, the cleavable ceDNA vectors as disclosed herein may be administered to treat, ameliorate, or prevent diseases of the CNS, including genetic disorders, neurodegenerative disorders, psychiatric disorders and tumors. Illustrative diseases of the CNS include, but are not limited to Alzheimer's disease. Parkinson's disease. Huntington's disease, Canavan disease, Leigh's disease, Refsum disease. Tourette syndrome, primary lateral sclerosis, amyotrophic lateral sclerosis, progressive muscular atrophy, Pick's disease, muscular dystrophy, multiple sclerosis, myasthenia gravis, Binswanger's disease, trauma due to spinal cord or head injury, Tay Sachs disease, U-sch-Nyan disease, epilepsy, cerebral infarcts, psychiatric disorders including mood disorders (e.g., depression, bipolar affective disorder, persistent affective disorder, secondary mood disorder), schizophrenia, drug dependency (e.g., alcoholism and other substance dependencies), neuroses (e.g., anxiety, obsessional disorder, somatoform disorder, dissociative disorder, grief, post-partum depression), psychosis (e.g., hallucinations and delusions), dementia, paranoia, attention deficit disorder, psychosexual disorders, sleeping disorders, pain disorders, eating or weight disorders (e.g., obesity, cachexia, anorexia nervosa, and bulimia) and cancers and tumors (e.g., pituitary tumors) of the CNS.

Ocular disorders that may be treated, ameliorated, or prevented with the cleavable ceDNA vectors of the invention include ophthalmic disorders involving the retina, posterior tract, and optic nerve (e.g., retinitis pigmentosa, diabetic retinopathy and other retinal degenerative diseases, uveitis, age-related macular degeneration, glaucoma). Many ophthalmic diseases and disorders are associated with one or more of three types of indications: (1) angiogenesis, (2) inflammation, and (3) degeneration. In some embodiments, the cleavable ceDNA vector as disclosed herein can be employed to deliver anti-angiogenic factors; anti-inflammatory factors; factors that retard cell degeneration, promote cell sparing, or promote cell growth and combinations of the foregoing. Diabetic retinopathy, for example, is characterized by angiogenesis. Diabetic retinopathy can be treated by delivering one or more anti-angiogenic factors either intraocularly (e.g., in the vitreous) or periocularly (e.g., in the sub-Tenon's region). One or more neurotrophic factors may also be co-delivered, either intraocularly (e.g., intravitreally) or periocularly. Additional ocular diseases that may be treated, ameliorated, or prevented with the cleavable ceDNA vectors of the invention include geographic atrophy, vascular or “wet” macular degeneration, Stargardt disease, Leber Congenital Amaurosis (LCA), Usher syndrome, pseudoxanthoma elasticum (PXE), x-linked retinitis pigmentosa (XLRP), x-linked retinoschisis (XLRS), Choroideremia, Leber hereditary optic neuropathy (LHON), Archomatopsia, cone-rod dystrophy, Fuchs endothelial corneal dystrophy, diabetic macular edema and ocular cancer and tumors.

In some embodiments, inflammatory ocular diseases or disorders (e.g., uveitis) can be treated, ameliorated, or prevented by the cleavable ceDNA vectors of the invention. One or more anti-inflammatory factors can be expressed by intraocular (e.g., vitreous or anterior chamber) administration of the cleavable ceDNA vector as disclosed herein. In other embodiments, ocular diseases or disorders characterized by retinal degeneration (e.g., retinitis pigmentosa) can be treated, ameliorated, or prevented by the cleavable ceDNA vectors of the invention. Intraocular (e.g., vitreal) administration of the cleavable ceDNA vector as disclosed herein encoding one or more neurotrophic factors can be used to treat such retinal degeneration-based diseases. In some embodiments, diseases or disorders that involve both angiogenesis and retinal degeneration (e.g., age-related macular degeneration) can be treated with the cleavable ceDNA vectors of the invention. Age-related macular degeneration can be treated by administering the cleavable ceDNA vector as disclosed herein encoding one or more neurotrophic factors intraocularly (e.g., vitreous) and/or one or more anti-angiogenic factors intraocularly or periocularly (e.g., in the sub-Tenon's region). Glaucoma is characterized by increased ocular pressure and loss of retinal ganglion cells. Treatments for glaucoma include administration of one or more neuroprotective agents that protect cells from excitotoxic damage using the cleavable ceDNA vector as disclosed herein. Accordingly, such agents include N-methyl-D-aspartate (NMDA) antagonists, cytokines, and neurotrophic factors, can be delivered intraocularly, optionally intravitreally using the cleavable ceDNA vector as disclosed herein.

In other embodiments, the cleavable ceDNA vector as disclosed herein may be used to treat seizures, e.g., to reduce the onset, incidence or severity of seizures. The efficacy of a therapeutic treatment for seizures can be assessed by behavioral (e.g., shaking, ticks of the eye or mouth) and/or electrographic means (most seizures have signature electrographic abnormalities). Thus, the cleavable ceDNA vector as disclosed herein can also be used to treat epilepsy, which is marked by multiple seizures over time. In one representative embodiment, somatostatin (or an active fragment thereof) is administered to the brain using the cleavable ceDNA vector as disclosed herein to treat a pituitary tumor. According to this embodiment, the cleavable ceDNA vector as disclosed herein encoding somatostatin (or an active fragment thereof) is administered by microinfusion into the pituitary. Likewise, such treatment can be used to treat acromegaly (abnormal growth hormone secretion from the pituitary). The nucleic acid (e.g., GenBank Accession No. J00306) and amino acid (e.g., GenBank Accession No. P01166; contains processed active peptides somatostatin-28 and somatostatin-14) sequences of somatostatins as are known in the art. In particular embodiments, the cleavable ceDNA vector can encode a transgene that comprises a secretory signal as described in U.S. Pat. No. 7,071,172.

Another aspect of the invention relates to the use of a cleavable ceDNA vector as described herein to produce antisense RNA, RNAi or other functional RNA (e.g., a ribozyme) for systemic delivery to a subject in vivo. Accordingly, in some embodiments, the cleavable ceDNA vector can comprise a transgene that encodes an antisense nucleic acid, a ribozyme (e.g., as described in U.S. Pat. No. 5,877,022), RN As that affect spliceosome-mediated trans-splicing (see, Puttaraju et al., (1999) Nature Biotech. 17:246; U.S. Pat. Nos. 6,013,487; 6,083,702), interfering RNAs (RNAi) that mediate gene silencing (see, Sharp et al., (2000) Science 287:2431) or other non-translated RNAs, such as “guide” RNAs (Gorman et al., (1998) Proc. Nat. Acad. Sci. USA 95:4929: U.S. Pat. No. 5,869,248 to Yuan et al.), and the like.

In some embodiments, the cleavable ceDNA vector can further also comprise a transgene that encodes a reporter polypeptide (e.g., an enzyme such as Green Fluorescent Protein, or alkaline phosphatase). In sonic embodiments, a transgene that encodes a reporter protein useful for experimental or diagnostic purposes, is selected from any of: β-lactamase, β-galactosidase (LacZ), alkaline phosphatase, thymidine kinase, green fluorescent protein (GFP), chloramphenicol acetyltransferase (CAT), luciferase, and others well known in the art. In some aspects, ceDNA vectors comprising a transgene encoding a reporter polypeptide may be used for diagnostic purposes or as markers of the cleavable ceDNA vector's activity in the subject to which they are administered.

In some embodiments, the cleavable ceDNA vector can comprise a transgene or a heterologous nucleotide sequence that shares homology with, and recombines with a locus on the host chromosome. This approach may be utilized to correct a genetic defect in the host cell.

In some embodiments, the cleavable ceDNA vector can comprise a transgene that can be used to express an immunogenic polypeptide in a subject, e.g., for vaccination. The transgene may encode any immunogen of interest known in the art including, but not limited to, immunogens from human immunodeficiency virus, influenza virus, gag proteins, tumor antigens, cancer antigens, bacterial antigens, viral antigens, and the like.

### D. Testing for Successful Gene Editing

Assays well known in the art can be used to test the efficiency of gene editing in both in vitro and in vivo models. Knock-in or knock-out of a desired transgene by a gene editing system comprising a ceDNA can be assessed by one skilled in the art by measuring mRNA and protein levels of the desired transgene (e.g., reverse transcription PCR, western blot analysis, and enzyme-linked immunosorbent assay (ELISA)). Nucleic acid alterations (e.g., point mutations, or deletion of DNA regions) can be assessed by deep sequencing of genomic target DNA. In one embodiment, ceDNA comprises a reporter protein that can be used to assess the expression of the desired transgene, for example by examining the expression of the reporter protein by fluorescence microscopy or a luminescence plate reader. For in vivo applications, protein function assays can be used to test the functionality of a given gene and/or gene product to determine if gene editing has successfully occurred. For example, it is envisioned that a point mutation in the cystic fibrosis transmembrane conductance regulator gene (CFTR) inhibits the capacity of CFTR to move anions (e.g., Cl⁻) through the anion channel, can be corrected by ceDNA's gene editing capacity. Following administration of ceDNA, one skilled in the art can assess the capacity for anions to move through the anion channel to determine if the point mutation of CFTR has been corrected. One skilled will be able to determine the best test for measuring functionality of a protein in vitro or in vivo.

It is contemplated herein that the effects of gene editing in a cell or subject can last for at least 1 month, at least 2 months, at least 3 months, at least four months, at least 5 months, at least six months, at least 10 months, at least 12 months, at least 18 months, at least 2 years, at least 5 years, at least 10 years, at least 20 years, or can be permanent.

In some embodiments, a transgene in the expression cassette, expression construct, or ceDNA vector described herein can be codon optimized for the host cell. As used herein, the term “codon optimized” or “codon optimization” refers to the process of modifying a nucleic acid sequence for enhanced expression in the cells of the vertebrate of interest, e.g., mouse or human (e.g., humanized), by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using e.g., Aptagen's Gene Forge® codon optimization and custom gene synthesis platform (Aptagen. Inc.) or another publicly available database.

### XIII. Administration

In particular embodiments, more than one administration (e.g., two, three, four or more administrations) may be employed to achieve the desired level of gene expression over a period of various intervals, e.g., daily, weekly, monthly, yearly, etc.

Exemplary modes of administration of the cleavable ceDNA vector disclosed herein includes oral, rectal, transmucosal, intranasal, inhalation (e.g., via an aerosol), buccal (e.g., sublingual), vaginal, intrathecal, intraocular, transdermal, intraendothelial, in utero (or in ovo), parenteral (e.g., intravenous, subcutaneous, intradermal, intracranial, intramuscular [including administration to skeletal, diaphragm and/or cardiac muscle], intrapleural, intracerebral, and intraarticular), topical (e.g., to both skin and mucosal surfaces, including airway surfaces, and transdermal administration), intralymphatic, and the like, as well as direct tissue or organ injection (e.g., to liver, eye, skeletal muscle, cardiac muscle, diaphragm muscle or brain).

Administration of the ceDNA vector can be to any site in a subject, including, without limitation, a site selected from the group consisting of the brain, a skeletal muscle, a smooth muscle, the heart, the diaphragm, the airway epithelium, the liver, the kidney, the spleen, the pancreas, the skin, and the eye. Administration of the ceDNA vector can also be to a tumor (e.g., in or near a tumor or a lymph node). The most suitable route in any given case will depend on the nature and severity of the condition being treated, ameliorated, and/or prevented and on the nature of the particular ceDNA vector that is being used. Additionally, ceDNA permits one to administer more than one transgene in a single vector, or multiple ceDNA vectors (e.g., a cleavable ceDNA cocktail).

Administration of the ceDNA vector disclosed herein to skeletal muscle according to the present invention includes but is not limited to administration to skeletal muscle in the limbs (e.g., upper arm, lower arm, upper leg, and/or lower leg), back, neck, head (e.g., tongue), thorax, abdomen, pelvis/perineum, and/or digits. The ceDNA as disclosed herein vector can be delivered to skeletal muscle by intravenous administration, intra-arterial administration, intraperitoneal administration, limb perfusion, (optionally, isolated limb perfusion of a leg and/or arm; see, e.g., Arruda et al., (2005) Blood 105: 3458-3464), and/or direct intramuscular injection. In particular embodiments, the ceDNA vector as disclosed herein is administered to a limb (arm and/or leg) of a subject (e.g., a subject with muscular dystrophy such as DMD) by limb perfusion, optionally isolated limb perfusion (e.g., by intravenous or intra-articular administration. In certain embodiments, the ceDNA vector as disclosed herein can be administered without employing “hydrodynamic” techniques.

Administration of the cleavable ceDNA vector as disclosed herein to cardiac muscle includes administration to the left atrium, right atrium, left ventricle, right ventricle and/or septum. The cleavable ceDNA vector as described herein can be delivered to cardiac muscle by intravenous administration, intra-arterial administration such as intra-aortic administration, direct cardiac injection (e.g., into left atrium, right atrium, left ventricle, right ventricle), and/or coronary artery perfusion. Administration to diaphragm muscle can be by any suitable method including intravenous administration, intra-arterial administration, and/or intra-peritoneal administration. Administration to smooth muscle can be by any suitable method including intravenous administration, intra-arterial administration, and/or intra-peritoneal administration. In one embodiment, administration can be to endothelial cells present in, near, and/or on smooth muscle.

In some embodiments, a cleavable ceDNA vector according to the present invention is administered to skeletal muscle, diaphragm muscle and/or cardiac muscle (e.g., to treat, ameliorate and/or prevent muscular dystrophy or heart disease (e.g., PAD or congestive heart failure).

### A. Ex Vivo Treatment

In some embodiments, cells are removed from a subject, a cleavable ceDNA vector is introduced therein, and the cells are then replaced back into the subject. Methods of removing cells from subject for treatment ex vivo, followed by introduction back into the subject are known in the art (see, e.g., U.S. Pat. No. 5,399,346; the disclosure of which is incorporated herein in its entirety). Alternatively, a cleavable ceDNA vector is introduced into cells from another subject, into cultured cells, or into cells from any other suitable source, and the cells are administered to a subject in need thereof.

Cells transduced with a cleavable ceDNA vector are preferably administered to the subject in a “therapeutically-effective amount” in combination with a pharmaceutical carrier. Those skilled in the art will appreciate that the therapeutic effects need not be complete or curative, as long as some benefit is provided to the subject.

In some embodiments, the cleavable ceDNA vector can encode a transgene (sometimes called a heterologous nucleotide sequence) that is any polypeptide that is desirably produced in a cell in vitro, ex vivo, or in vivo. For example, in contrast to the use of the cleavable ceDNA vectors in a method of treatment as discussed herein, in some embodiments the cleavable ceDNA vectors may be introduced into cultured cells and the expressed gene product isolated therefrom, e.g., for the production of antigens or vaccines.

The cleavable ceDNA vectors can be used in both veterinary and medical applications. Suitable subjects for ex vivo gene delivery methods as described above include both avians (e.g., chickens, ducks, geese, quail, turkeys and pheasants) and mammals (e.g., humans, bovines, ovines, caprines, equines, felines, canines, and lagomorphs), with mammals being preferred. Human subjects are most preferred. Human subjects include neonates, infants, juveniles, and adults.

One aspect of the technology described herein relates to a method of delivering a transgene to a cell. Typically, for in vitro methods, the cleavable ceDNA vector may be introduced into the cell using the methods as disclosed herein, as well as other methods known in the art. ceDNA vectors disclosed herein are preferably administered to the cell in a biologically-effective amount. If the cleavable ceDNA vector is administered to a cell in vivo (e.g., to a subject), a biologically-effective amount of the cleavable ceDNA vector is an amount that is sufficient to result in transduction and expression of the transgene in a target cell.

### B. Dose Ranges

In vivo and/or in vitro assays can optionally be employed to help identify optimal dosage ranges for use. The precise dose to be employed in the formulation will also depend on the route of administration, and the seriousness of the condition, and should be decided according to the judgment of the person of ordinary skill in the art and each subject's circumstances. Effective doses can be extrapolated from dose-response curves derived from in vitro or animal model test systems.

A cleavable ceDNA vector is administered in sufficient amounts to transfect the cells of a desired tissue and to provide sufficient levels of gene transfer and expression without undue adverse effects. Conventional and pharmaceutically acceptable routes of administration include, but are not limited to, those described above in the “Administration” section, such as direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration can be combined, if desired.

The dose of the amount of a cleavable ceDNA vector required to achieve a particular “therapeutic effect.” will vary based on several factors including, but not limited to: the route of nucleic acid administration, the level of gene or RNA expression required to achieve a therapeutic effect, the specific disease or disorder being treated, and the stability of the gene(s). RNA product(s), or resulting expressed protein(s). One of skill in the art can readily determine a cleavable ceDNA vector dose range to treat a patient having a particular disease or disorder based on the aforementioned factors, as well as other factors that are well known in the art.

Dosage regime can be adjusted to provide the optimum therapeutic response. For example, the oligonucleotide can be repeatedly administered, e.g., several doses can be administered daily or the dose can be proportionally reduced as indicated by the exigencies of the therapeutic situation. One of ordinary skill in the art will readily be able to determine appropriate doses and schedules of administration of the subject oligonucleotides, whether the oligonucleotides are to be administered to cells or to subjects.

A “therapeutically effective dose” will fall in a relatively broad range that can be determined through clinical trials and will depend on the particular application (neural cells will require very small amounts, while systemic injection would require large amounts). For example, for direct in vivo injection into skeletal or cardiac muscle of a human subject, a therapeutically effective dose will be on the order of from about 1 μg to 100 g of the cleavable ceDNA vector. If exosomes or microparticles are used to deliver the cleavable ceDNA vector, then a therapeutically effective dose can be determined experimentally, but is expected to deliver from 1 μg to about 100 g of vector. Moreover, a therapeutically effective dose is an amount that has an effect on editing the target gene that results in a reduction in one or more symptoms of the disease, but does not result in gene editing of off-target genes.

Formulation of pharmaceutically-acceptable excipients and carrier solutions is well-known to those of skill in the art, as is the development of suitable dosing and treatment regimens for using the particular compositions described herein in a variety of treatment regimens.

For in vitro transfection, an effective amount of a cleavable ceDNA vector to be delivered to cells (1×10⁶ cells) will be on the order of 0.1 to 100 μg ceDNA vector, preferably 1 to 20 μg, and more preferably 1 to 15 μg or 8 to 1 μg. Larger ceDNA vectors will require higher doses. If exosomes or microparticles are used, an effective in vitro dose can be determined experimentally but would be intended to deliver generally the same amount of the cleavable ceDNA vector.

Treatment can involve administration of a single dose or multiple doses. In some embodiments, more than one dose can be administered to a subject; in fact multiple doses can be administered as needed, because the cleavable ceDNA vector elicits does not elicit an anti-capsid host immune response due to the absence of a viral capsid. As such, one of skill in the art can readily determine an appropriate number of doses. The number of doses administered can, for example, be on the order of 1-100, preferably 2-20 doses.

Without wishing to be bound by any particular theory, the lack of typical anti-viral immune response elicited by administration of a cleavable ceDNA vector as described by the disclosure (i.e., the absence of capsid components) allows the cleavable ceDNA vector to be administered to a host on multiple occasions. In some embodiments, the number of occasions in which a heterologous nucleic acid is delivered to a subject is in a range of 2 to 10 times (e.g., 2, 3, 4, 5, 6, 7, 8, 9, or 10 times). In some embodiments, a cleavable ceDNA vector is delivered to a subject more than 10 times.

In some embodiments, a dose of a cleavable ceDNA vector is administered to a subject no more than once per calendar day (e.g., a 24-hour period). In some embodiments, a dose of a cleavable ceDNA vector is administered to a subject no more than once per 2, 3, 4, 5, 6, or 7 calendar days. In some embodiments, a dose of a cleavable ceDNA vector is administered to a subject no more than once per calendar week (e.g., 7 calendar days). In some embodiments, a dose of a cleavable ceDNA vector is administered to a subject no more than bi-weekly (e.g., once in a two calendar week period). In some embodiments, a dose of a cleavable ceDNA vector is administered to a subject no more than once per calendar month (e.g., once in 30 calendar days). In some embodiments, a dose of a cleavable ceDNA vector is administered to a subject no more than once per six calendar months. In some embodiments, a dose of a cleavable ceDNA vector is administered to a subject no more than once per calendar year (e.g., 365 days or 366 days in a leap year).

### C. Unit Dosage Forms

In some embodiments, the pharmaceutical compositions can conveniently be presented in unit dosage form. A unit dosage form will typically be adapted to one or more specific routes of administration of the pharmaceutical composition. In some embodiments, the unit dosage form is adapted for administration by inhalation. In some embodiments, the unit dosage form is adapted for administration by a vaporizer. In some embodiments, the unit dosage form is adapted for administration by a nebulizer. In some embodiments, the unit dosage form is adapted for administration by an aerosolizer. In some embodiments, the unit dosage form is adapted for oral administration, for buccal administration, or for sublingual administration. In some embodiments, the unit dosage form is adapted for intravenous, intramuscular, or subcutaneous administration. In some embodiments, the unit dosage form is adapted for intrathecal or intracerebroventricular administration. In some embodiments, the pharmaceutical composition is formulated for topical administration. The amount of active ingredient which can be combined with a carrier material to produce a single dosage form will generally be that amount of the compound which produces a therapeutic effect.

### XIV. Various Applications

The gene editing systems comprising cleavable ceDNA or cleaved ceDNA, a nuclease and at lest one gRNA, can be used for various purposes as described above. In some embodiments, the gene editing system targets a target gene, e.g., a protein or functional RNA, that is to be edited for research purposes, e.g., to create a somatic transgenic animal model harboring one or more mutations or a corrected gene sequence, e.g., to study the function of the target gene. In another example, the gene editing system is used to gene edit a target gene that encodes a protein or functional RNA to create an animal model of disease.

In some embodiments, the target gene of the gene editing system encodes one or more peptides, polypeptides, or proteins, which are useful for the treatment, amelioration, or prevention of disease states in a mammalian subject. The gene editing system can be transferred (e.g., expressed in) to a patient in a sufficient amount to treat a disease associated with an abnormal gene sequence, which can result in any one or more of the following: reduced expression, lack of expression or dysfunction of the target gene.

In some embodiments, the gene editing systems disclosed herein, which comprise a cleavable or cleaved ceDNA, are envisioned for use in diagnostic and screening methods, whereby a gene editing system is transiently or stably expressed in a cell culture system, or alternatively, a transgenic animal model.

Another aspect of the technology described herein provides a method of transducing a population of mammalian cells. In an overall and general sense, the method includes at least the step of introducing into one or more cells of the population, a composition that comprises an effective amount of one or more of the cleavable ceDNA disclosed herein.

Additionally, the present invention provides compositions, as well as therapeutic and/or diagnostic kits that include one or more of the disclosed ceDNA vectors or ceDNA compositions, formulated with one or more additional ingredients, or prepared with one or more instructions for their use.

A cell to be administered the cleavable ceDNA vector as disclosed herein may be of any type, including but not limited to neural cells (including cells of the peripheral and central nervous systems, in particular, brain cells), lung cells, retinal cells, epithelial cells (e.g., gut and respiratory epithelial cells), muscle cells, dendritic cells, pancreatic cells (including islet cells), hepatic cells, myocardial cells, bone cells (e.g., bone marrow stem cells), hematopoietic stem cells, spleen cells, keratinocytes, fibroblasts, endothelial cells, prostate cells, germ cells, and the like. Alternatively, the cell may be any progenitor cell. As a further alternative, the cell can be a stein cell (e.g., neural stem cell, liver stem cell). As still a further alternative, the cell may be a cancer or tumor cell. Moreover, the cells can be from any species of origin, as indicated above.

### EXAMPLES

The following examples are provided by way of illustration not limitation. It will be appreciated by one of ordinary skill in the art that ceDNA vectors can be constructed from any of the wild-type or modified ITRs described herein, and that the following exemplary methods can be used to construct and assess the activity of such ceDNA vectors. While the methods are exemplified with certain ceDNA vectors, they are applicable to any ceDNA vector in keeping with the description.

### Example 1: Constructing Cleavable ceDNA Vectors

Production of ceDNA vectors using a polynucleotide construct template is described in Example 1 of PCT/US18/49996, and is incorporated by reference in its entirety herein. For example, a polynucleotide construct template used for generating the cleavable ceDNA vectors of the present invention can be a cleavable ceDNA-plasmid, a cleavable ceDNA-Bacmid, and/or a cleavable ceDNA-baculovirus. Without being limited to theory, in a permissive host cell, in the presence of e.g., Rep, the polynucleotide construct template having two symmetric ITRs and an expression construct, where at least one of the ITRs is modified relative to a wild-type ITR sequence, replicates to produce ceDNA vectors. ceDNA vector production undergoes two steps: first, excision (“rescue”) of template from the template backbone (e.g., ceDNA-plasmid, ceDNA-bacmid, ceDNA-baculovirus genome etc.) via Rep proteins, and second, Rep mediated replication of the excised ceDNA vector.

An exemplary method to produce ceDNA vectors is from a cleavable ceDNA-plasmid as described in PCT1 publication WO2019113310, incorporated by reference in its entirety herein. FIGS. 4A-4E of WO2019113310 show how to make a characterize ceDNA vectors from a plasmid.

Cleavable ceDNA plasmids are set forth below in Table 8. A corresponding standard, non-cleavable ceDNA is also shown (Plasmid: SP-210367; see Table 8).

TABLE 8

| wild-type ITR | Description |
|---|---|
| Cleavable ceDNA | GFP-gRNA1-TS + PAM-100 bp_spacers (stuffers) |
| 344 | |
| Cleavable ceDNA | GFP-gRNA2-TS + PAM-100 bp_spacers (stuffers) |
| 136 | |
| Cleavable ceDNA | GFP-gRNA3-TS + PAM-100 bp_spacers (stuffers) |
| 137 | |
| Cleavable ceDNA | GFP-gRNA4-TS + PAM-100 bp_spacers (stuffers) |
| 138 | |
| ceDNA 345 | GFP non-cleavable |
| Cleavable ceDNA | Bidirectional Luciferase gRNA1-TS + |
| 350 | PAM-orientation1 |
| Cleavable ceDNA | Bidirectional Luciferase- gRNA1-TS + |
| 351 | PAM-orientation2 |
| Cleavable ceDNA | Bidirectional Luciferase-gRNA2-TS + |
| 352 | PAM-orientation1 |
| Cleavable ceDNA | Bidirectional Luciferase-gRNA2-TS + |
| 353 | PAM-orientation2 |
| ceDNA 354 | Bidirectional Luciferase non-cleavable |
| ceDNA 355 | Bidirectional FIX non-cleavable |
| Cleavable ceDNA | Bidirectional FIX + sgRNA1-TS + PAM |
| 356 | |
| ceDNA 346 | GFP-mCherry non-cleavable |
| Cleavable ceDNA | GFP-mCherry-sgRNA1-TS + PAM |
| 347 | |
| Cleavable ceDNA | GFP-mCherry-sgRNA2-TS + PAM |
| 348 | |
| Cleavable ceDNA | GFP-mCherry-sgRNA4-TS + PAM |
| 349 | |
| Cleavable ceDNA | GFP-gRNA3-TS + PAM-no spacer (stuffer) |
| 449 | |
| Cleavable ceDNA | GFP-gRNA2-TS + PAM-no spacer (stuffer) |
| 448 | |

FIG. 3A shows the nucleic acid sequence of cleavable ceDNA 344 (SEQ ID NO: 27). As shown in FIG. 3B, the first (SEQ ID NO: 45) and second (SEQ ID NO: 46) spacer (stuffer) sequences are bolded, and the first and second sgRNA and PAM sequences (SEQ ID NO: 47 and SEQ ID NO: 48, respectively) are bolded and underlined.

FIG. 4A shows the nucleic acid sequence of cleavable ceDNA 136 (SEQ ID NO: 28). As shown in FIG. 4B, the first (SEQ ID NO: 49) and second (SEQ ID NO: 50) spacer (stuffer) sequences are bolded, and the first and second sgRNA and PAM sequences (SEQ ID NO: 51 and SEQ ID NO: 52, respectively) are bolded and underlined.

FIG. 5A shows the nucleic acid sequence of cleavable ceDNA 137 (SEQ ID NO: 29). As shown in FIG. 5B, the first (SEQ ID NO: 53) and second (SEQ ID NO: 54) spacer (stuffer) sequences are bolded, and the first and second sgRNA and PAM sequences (SEQ ID NO: 55 and SEQ ID NO: 56, respectively) are bolded and underlined.

FIG. 6A shows the nucleic acid sequence of cleavable ceDNA 138 (SEQ ID NO: 30). As shown in FIG. 6B, the first (SEQ ID NO: 57) and second (SEQ ID NO: 58) spacer (stuffer) sequences are bolded, and the first and second sgRNA and PAM sequences (SEQ ID NO: 59 and SEQ ID NO: 60, respectively) are bolded and underlined.

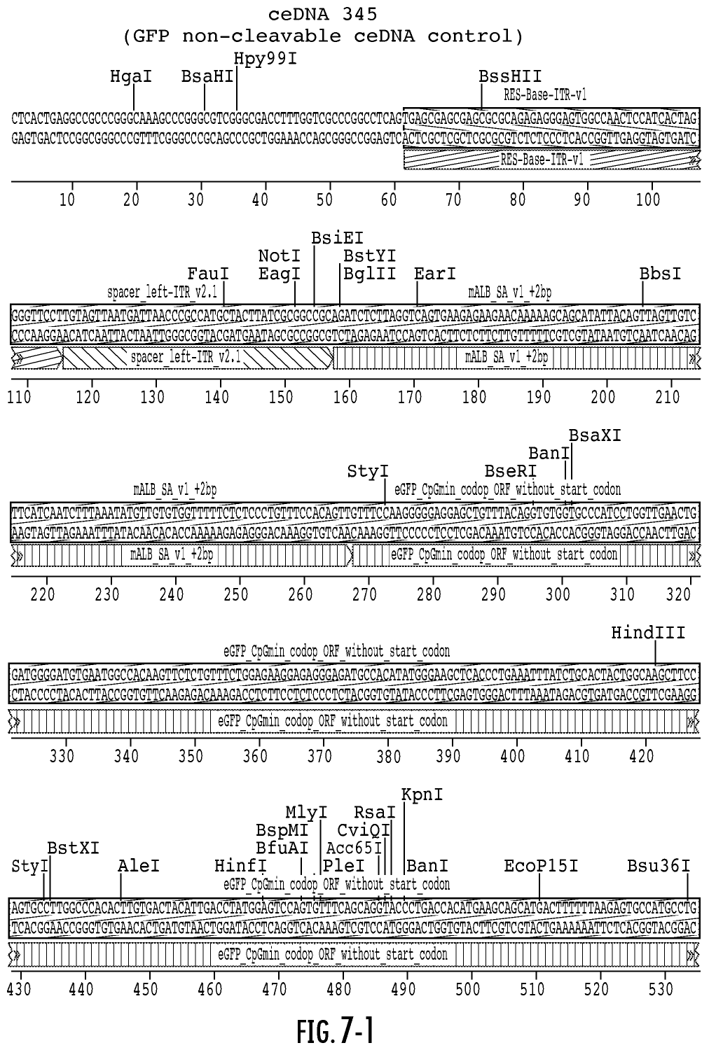

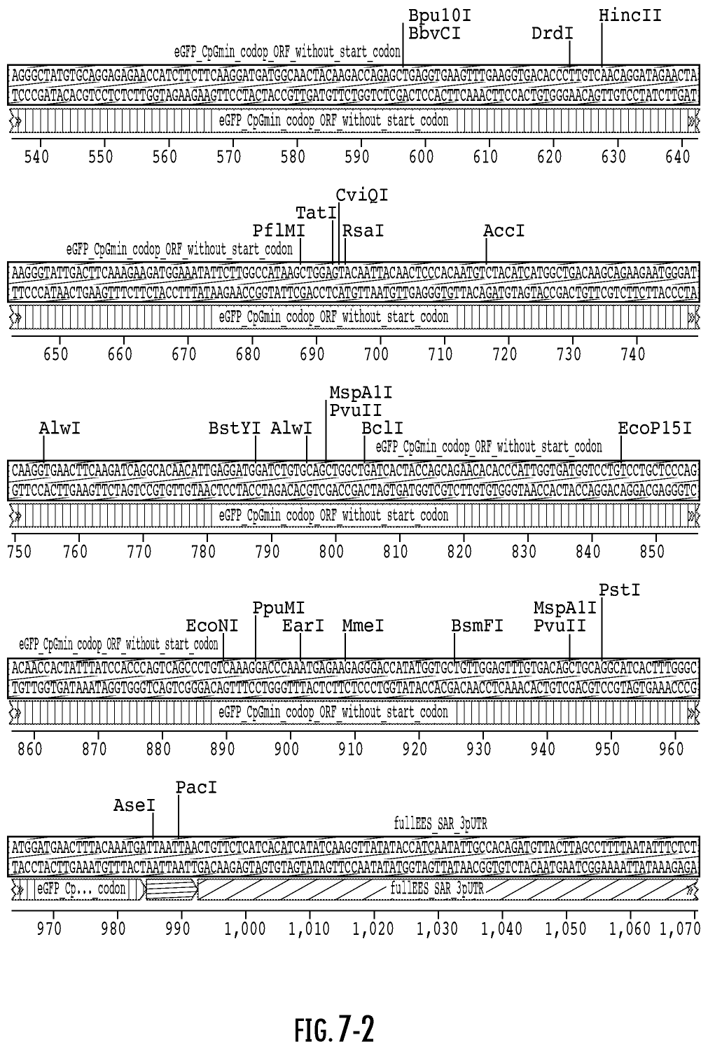

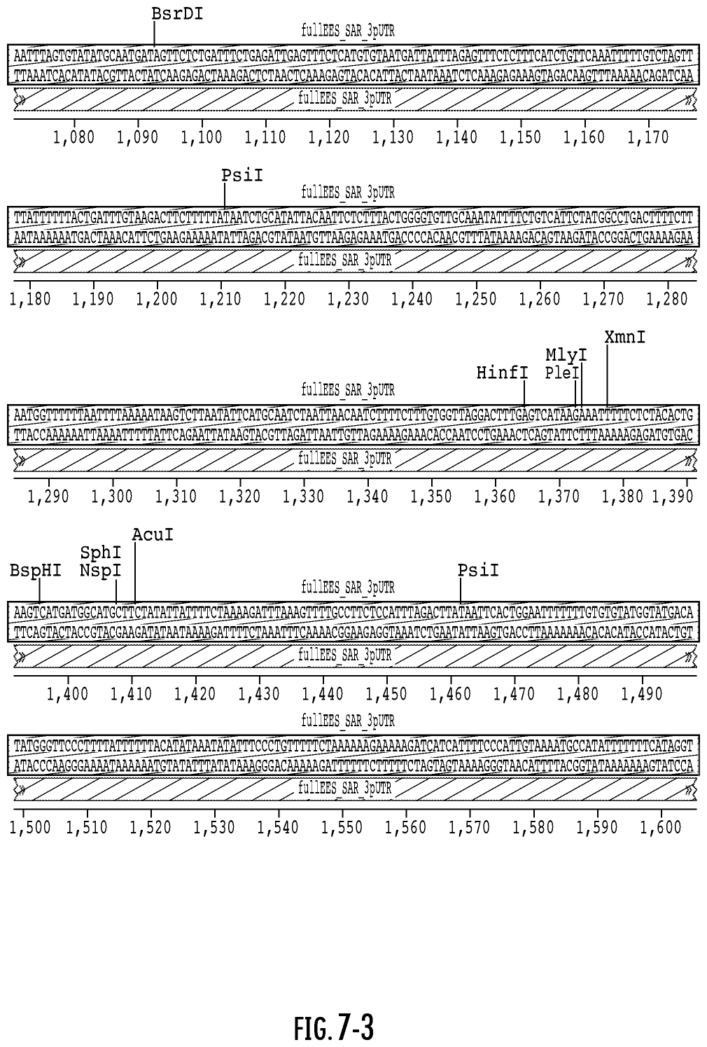

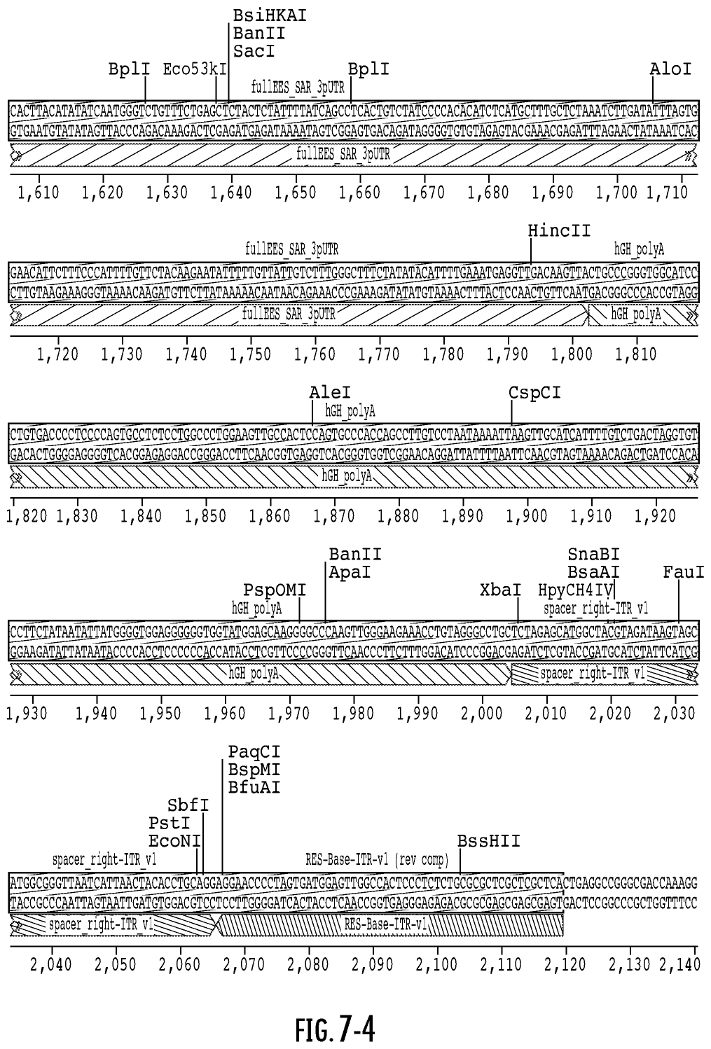

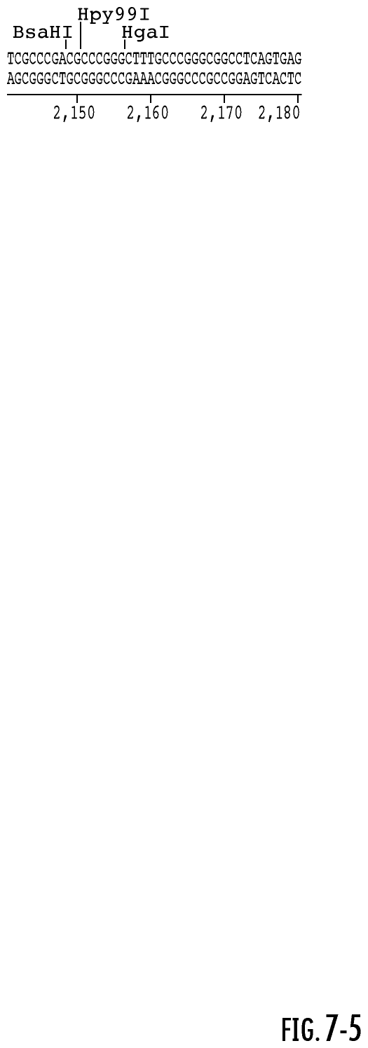

FIG. 7 shows the nucleic acid sequence of non-leavable ceDNA 345 (SEQ ID NO: 31) used as control.

FIG. 8A shows the nucleic acid sequence of cleavable ceDNA 350 (SEQ ID NO: 32). As shown in FIG. 8B, the first (SEQ ID NO: 61) and second (SEQ ID NO: 62) spacer (stuffer) sequences are bolded, and the first and second sgRNA and PAM sequences (SEQ ID NO: 63 and SEQ ID NO: 64, respectively) are bolded and underlined.

FIG. 9A shows the nucleic acid sequence of cleavable ceDNA 351 (SEQ ID NO: 33). As shown in FIG. 9B, the first (SEQ ID NO: 65) and second (SEQ ID NO: 66) spacer (stuffer) sequences are bolded, and the first and second sgRNA and PAM sequences (SEQ ID NO: 67 and SEQ ID NO: 68, respectively) are bolded and underlined.

FIG. 10A shows the nucleic acid sequence of cleavable ceDNA 352 (SEQ ID NO: 34). As shown in FIG. 10B, the first (SEQ ID NO: 69) and second (SEQ ID NO: 70) spacer (stuffer) sequences are bolded, and the first and second sgRNA and PAM sequences (SEQ ID NO: 71 and SEQ ID NO: 72, respectively) are bolded and underlined.

FIG. 11A shows the nucleic acid sequence of cleavable ceDNA 353(SEQ ID NO: 35). As shown in FIG. 11B, the first (SEQ ID NO: 73) and second (SEQ ID NO: 74) spacer (stuffer) sequences are bolded, and the first and second sgRNA and PAM sequences (SEQ ID NO: 75 and SEQ ID NO: 76, respectively) are bolded and underlined.

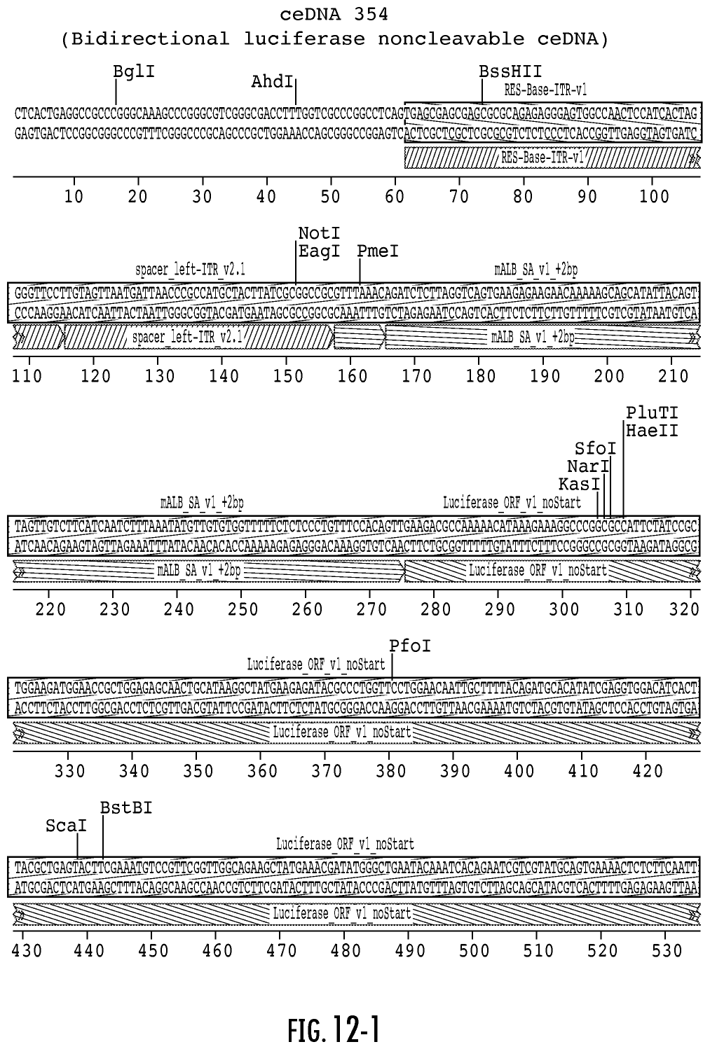

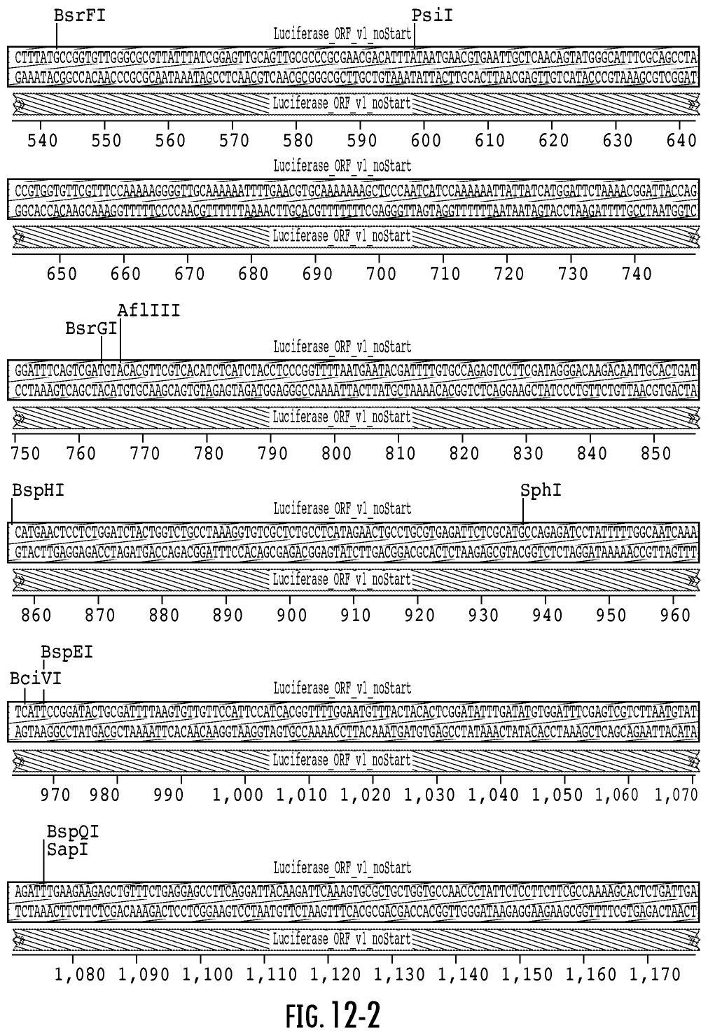

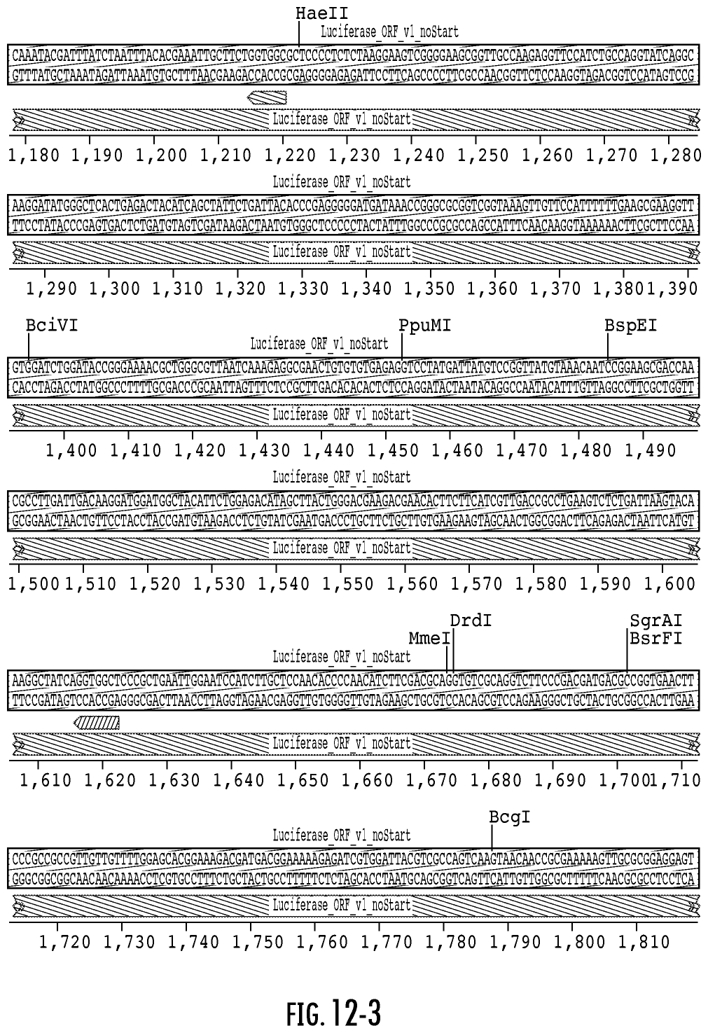

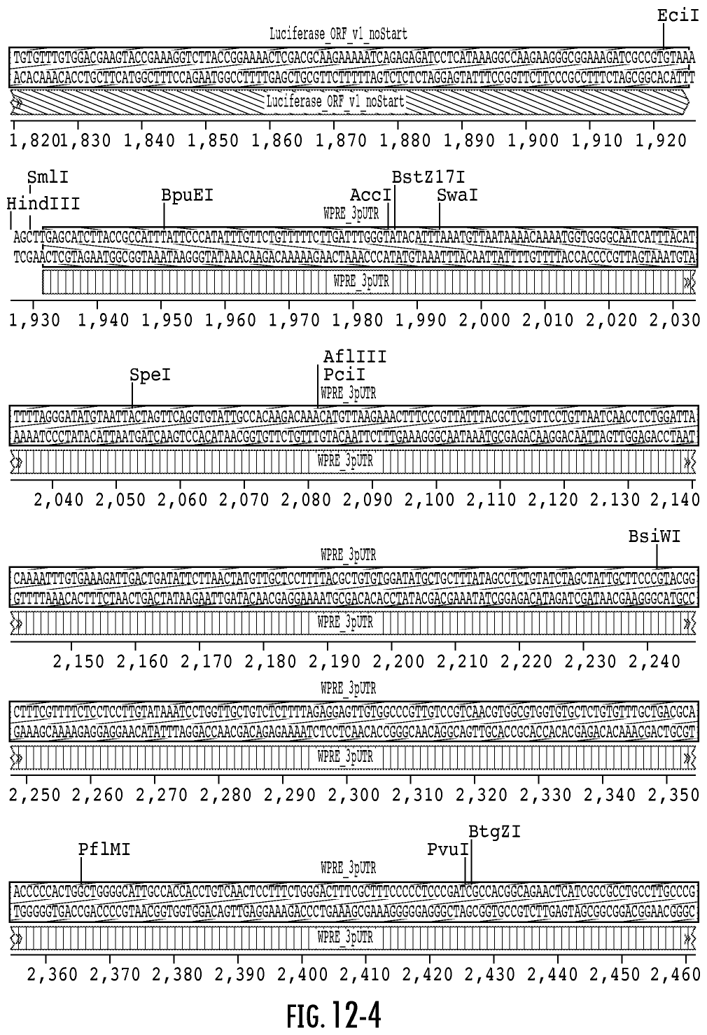

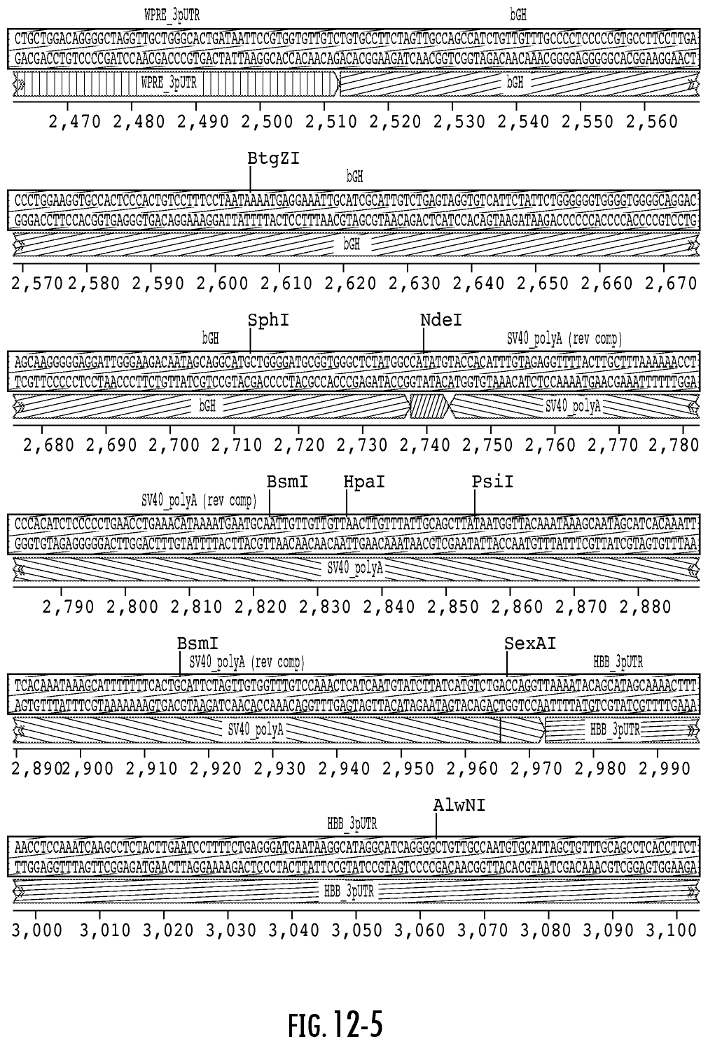

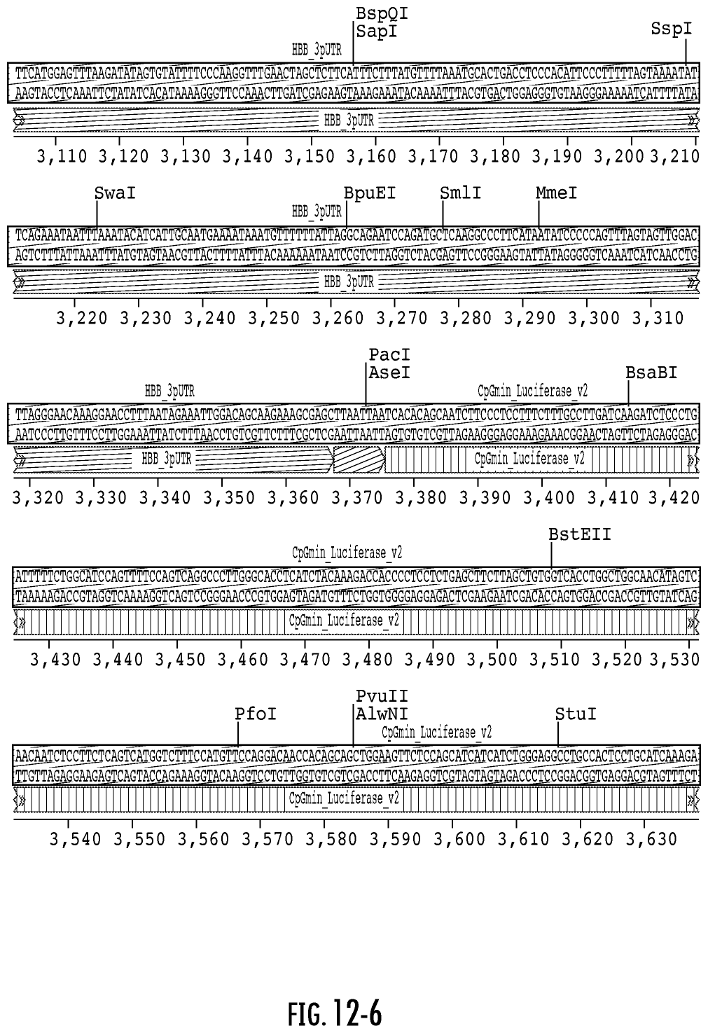

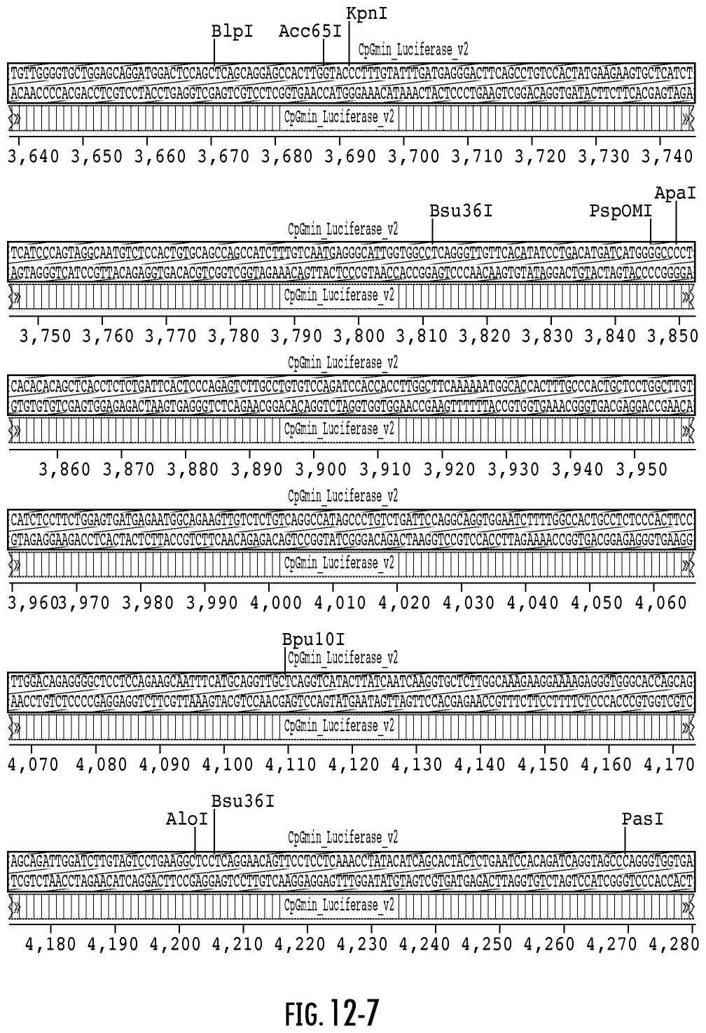

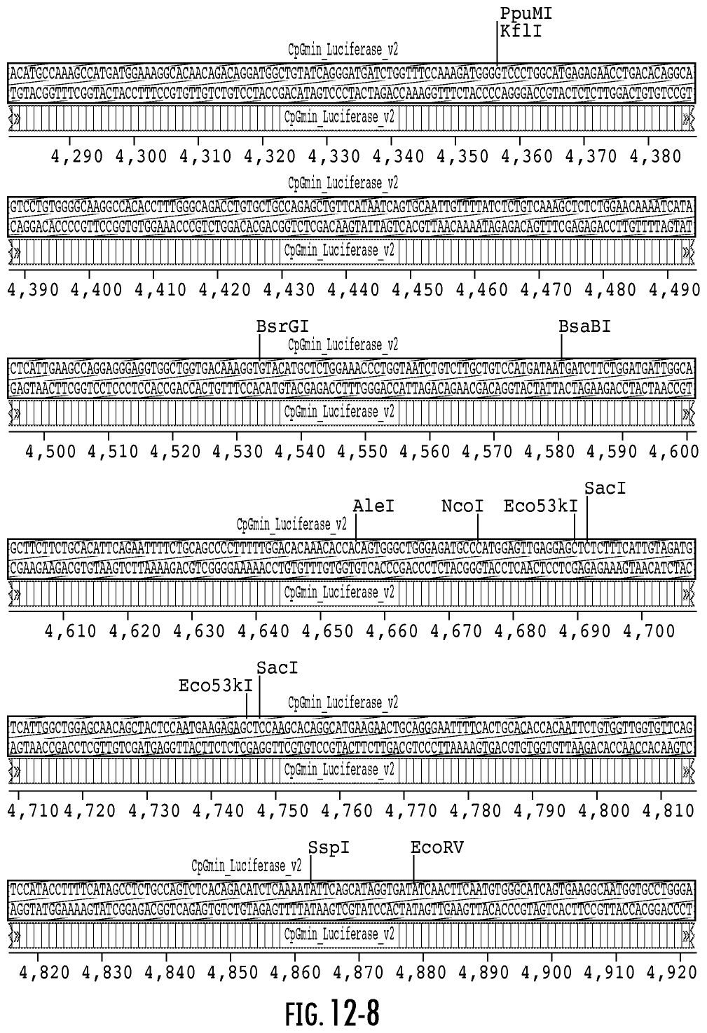

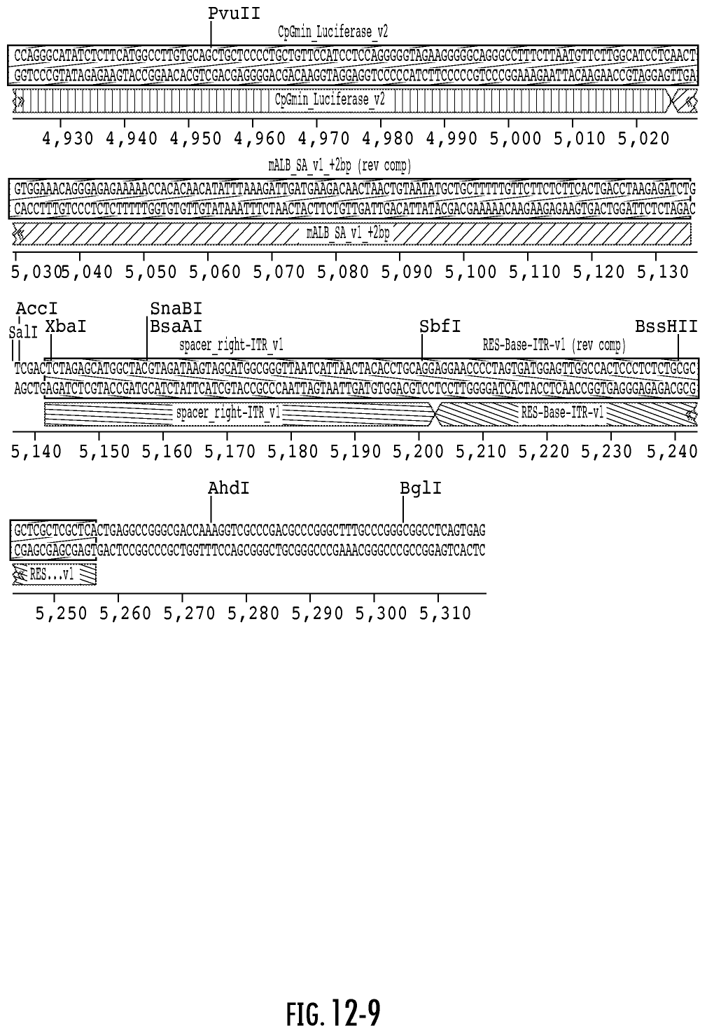

FIG. 12 shows the nucleic acid sequence of nonleavable ceDNA 354 (SEQ ID NO: 36) encoding birectional luciferase.

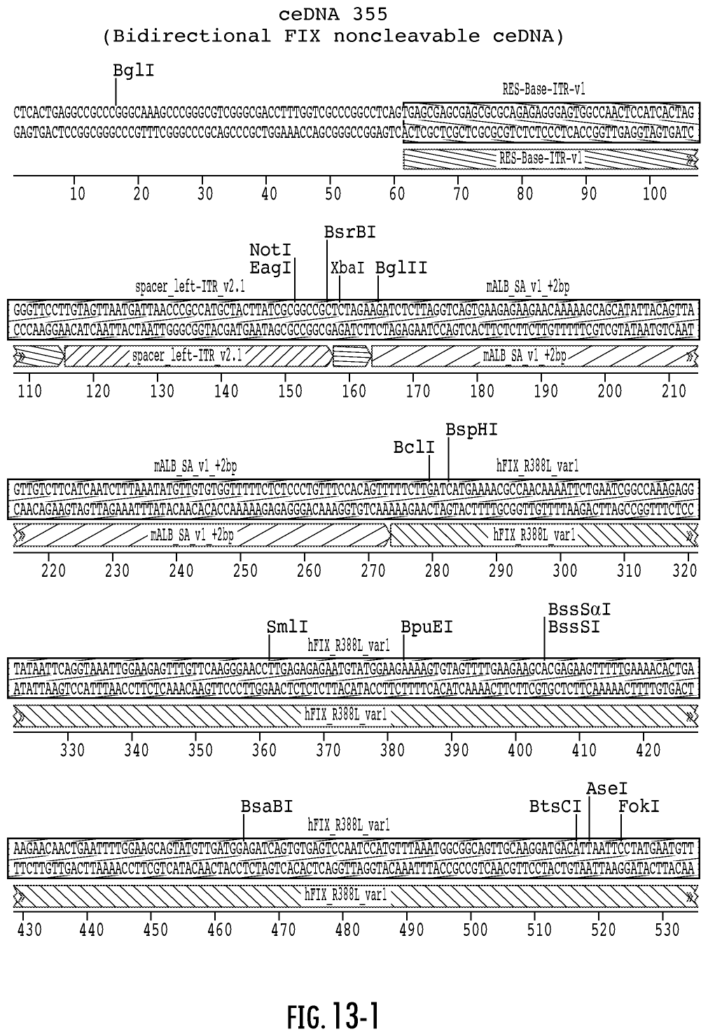

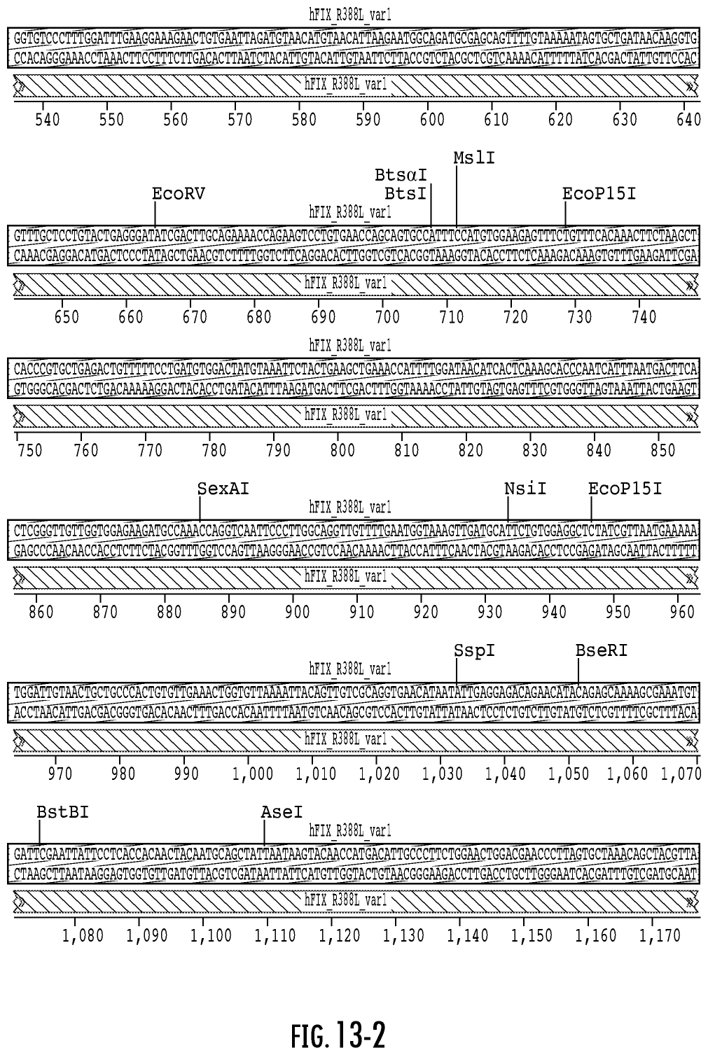

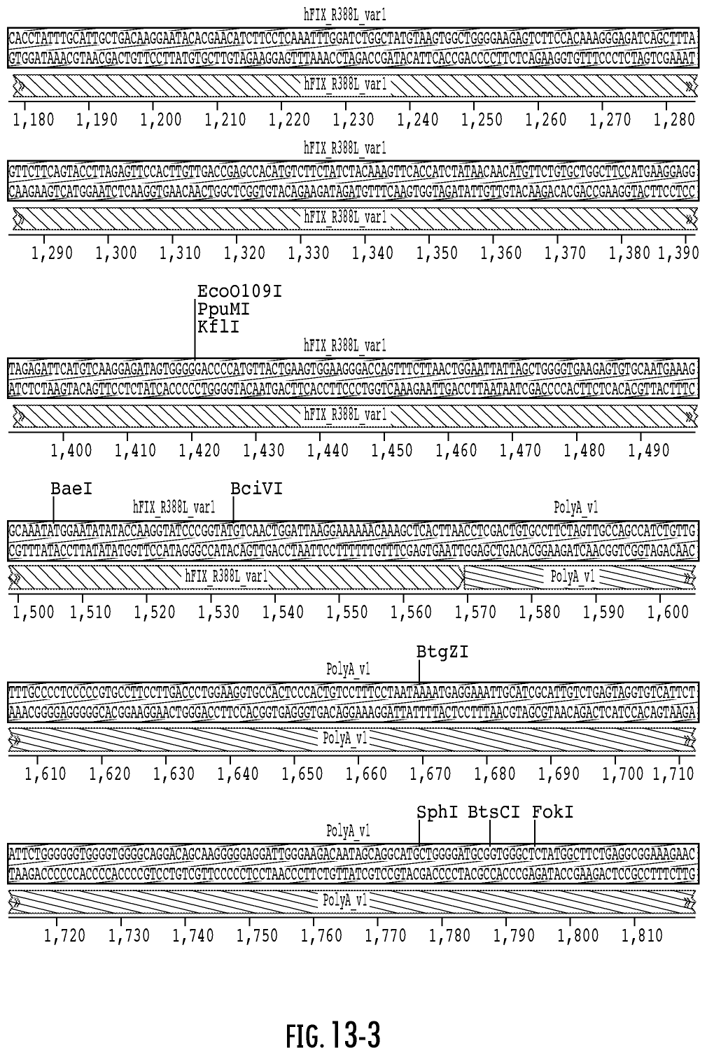

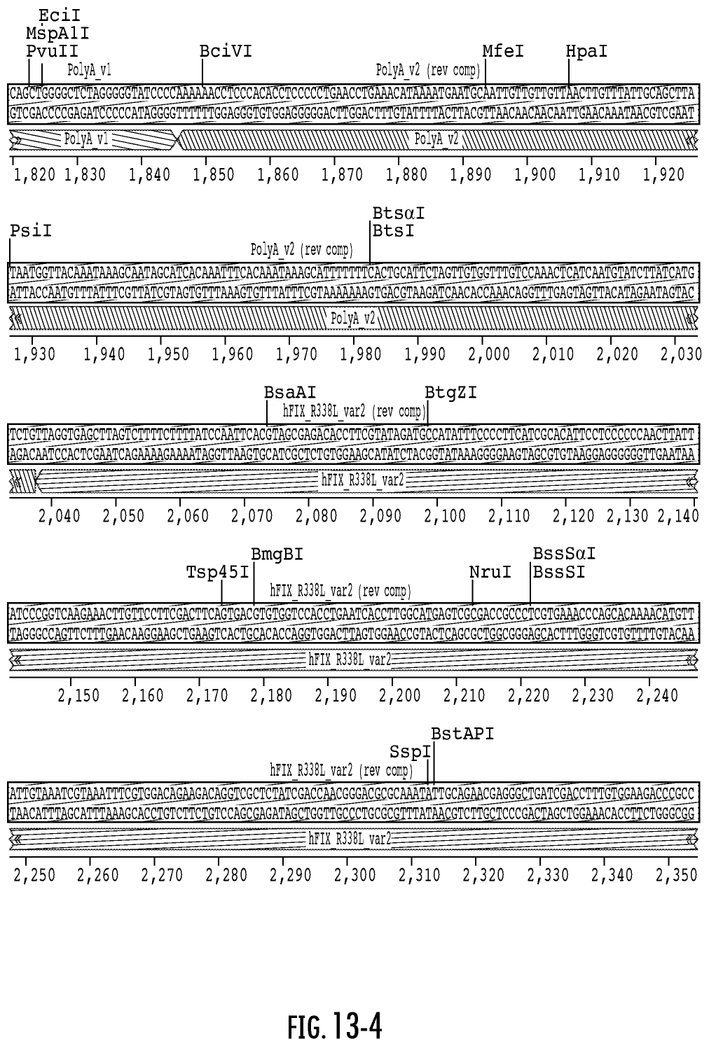

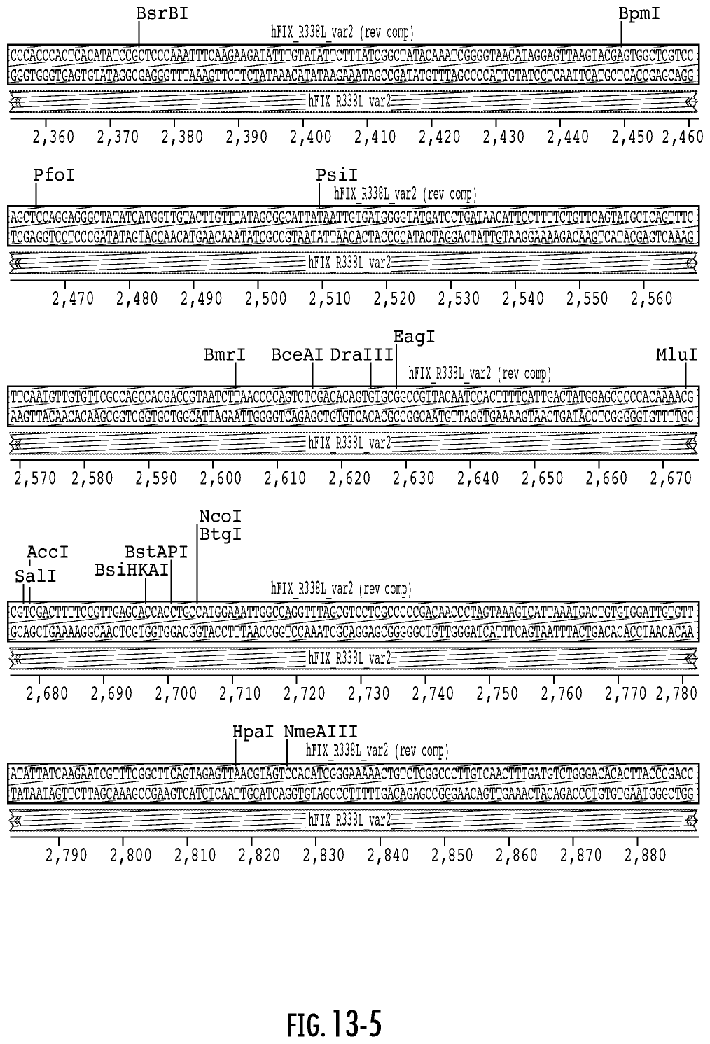

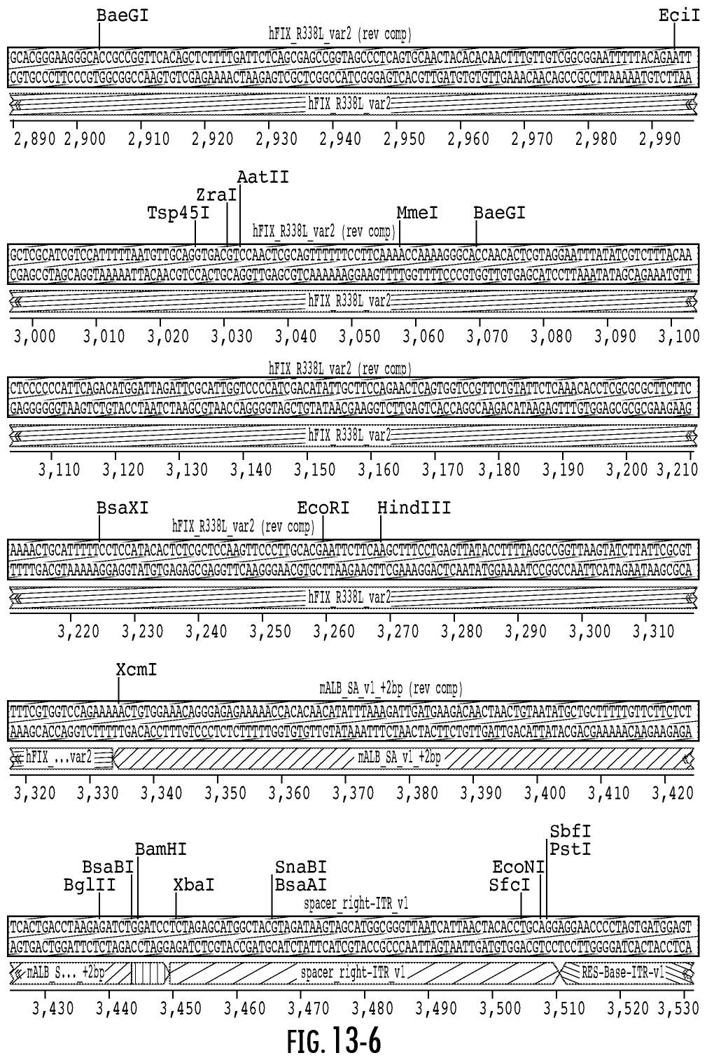

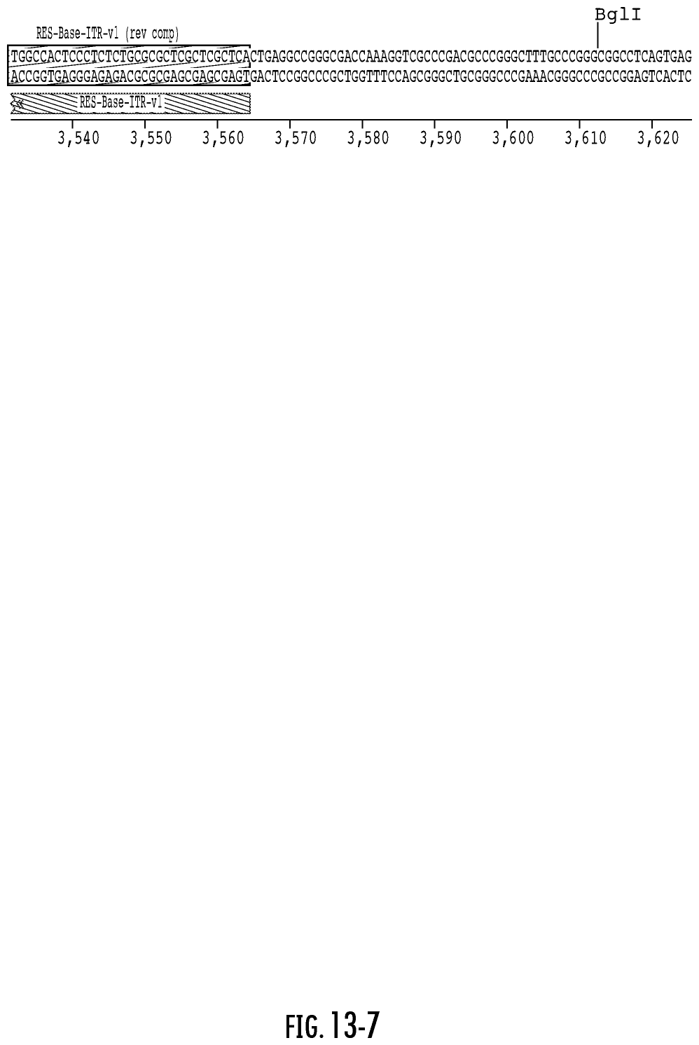

FIG. 13 shows the nucleic acid sequence of noncleavable ceDNA 355 (SEQ ID NO: 37) encoding bidirectional Factor IX (FIX).

FIG. 14A shows the nucleic acid sequence of cleavable ceDNA 356 (SEQ ID NO: 38). As shown in FIG. 14B, the first (SEQ ID NO: 77) and second (SEQ ID NO: 78) spacer (stuffer) sequences are bolded, and the first and second sgRNA and PAM sequences (SEQ ID NO: 79 and SEQ ID NO: 80, respectively) are bolded and underlined.

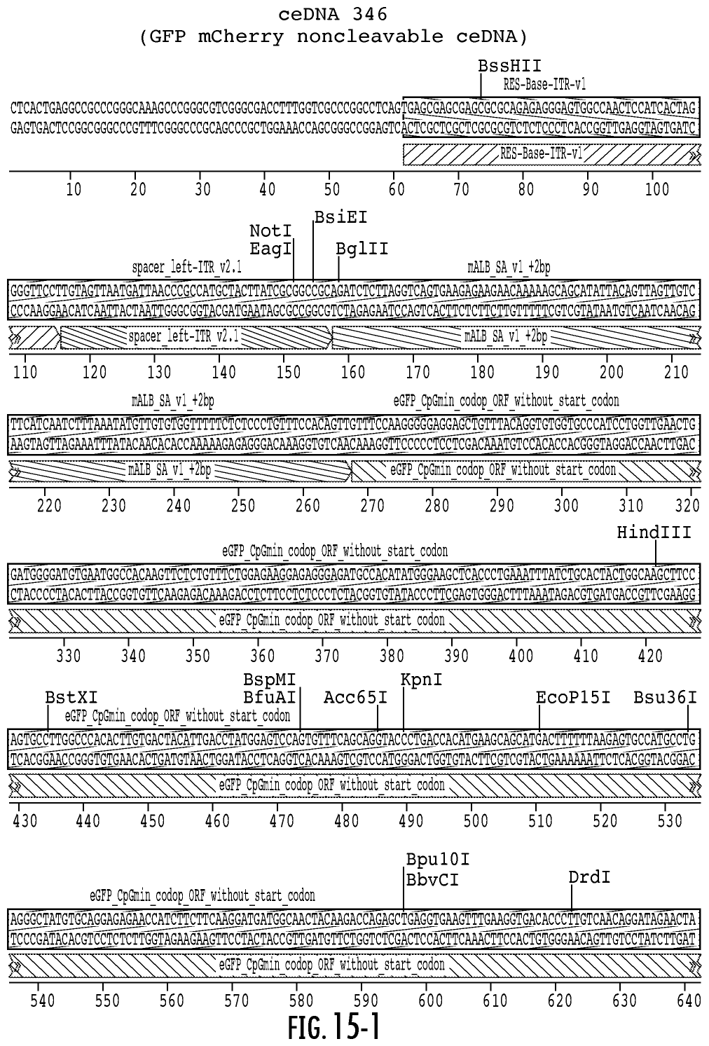

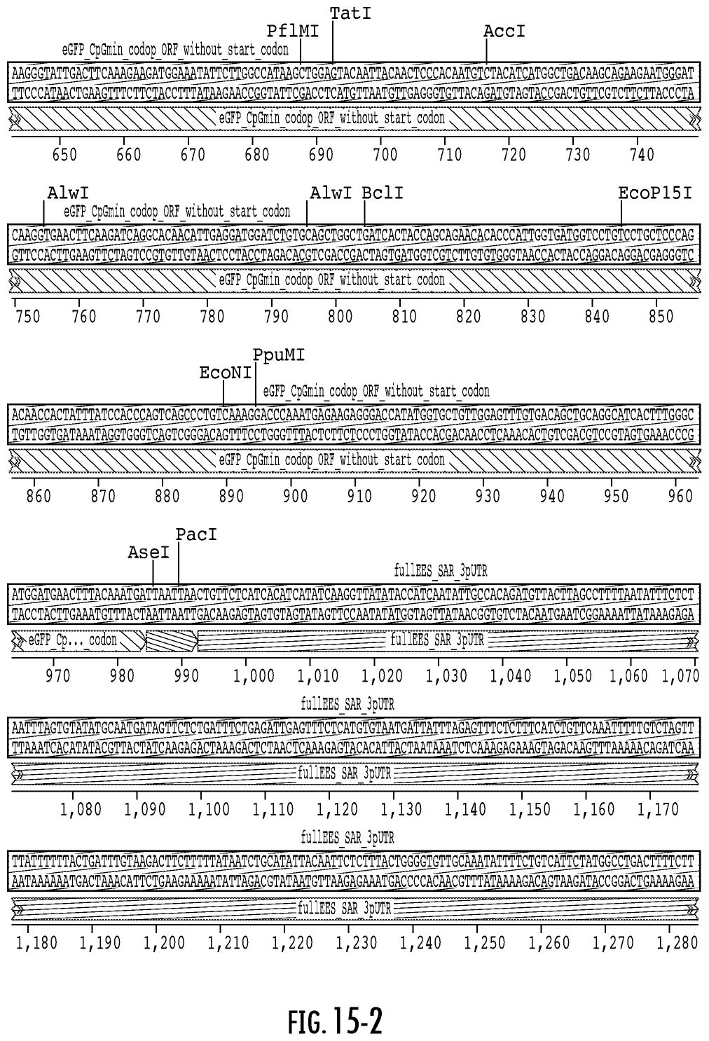

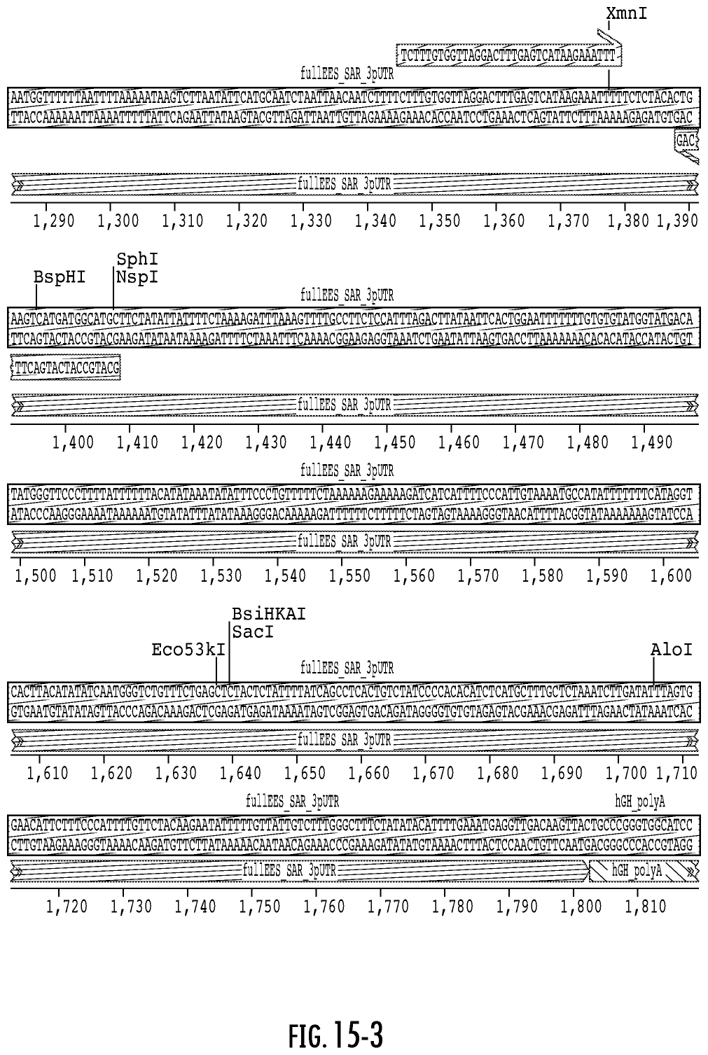

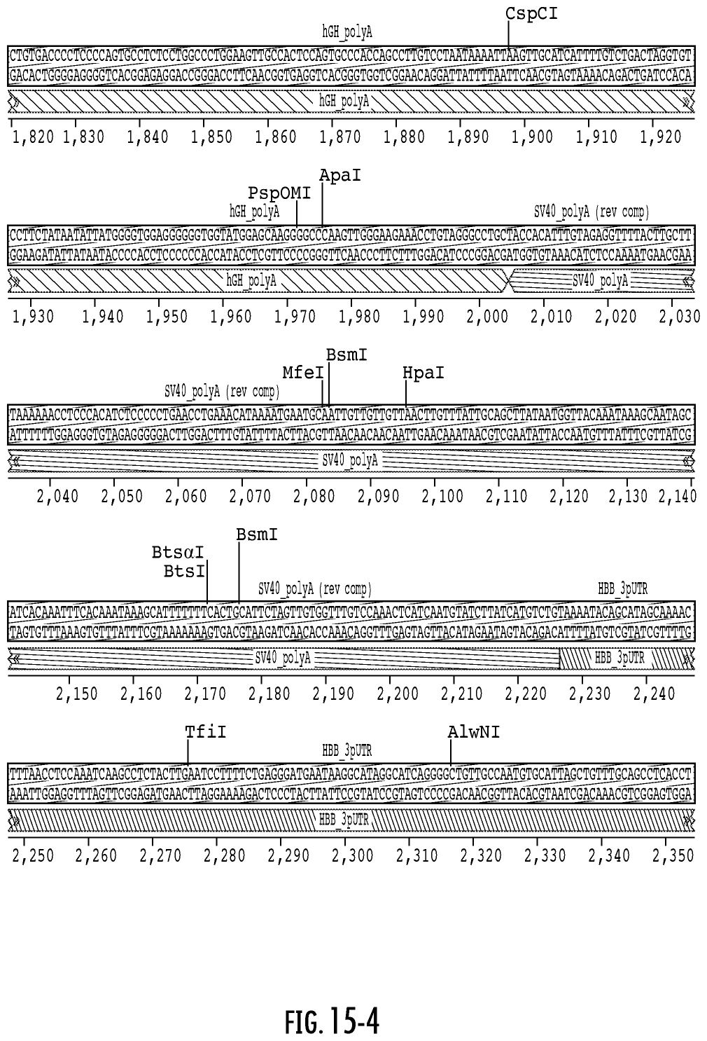

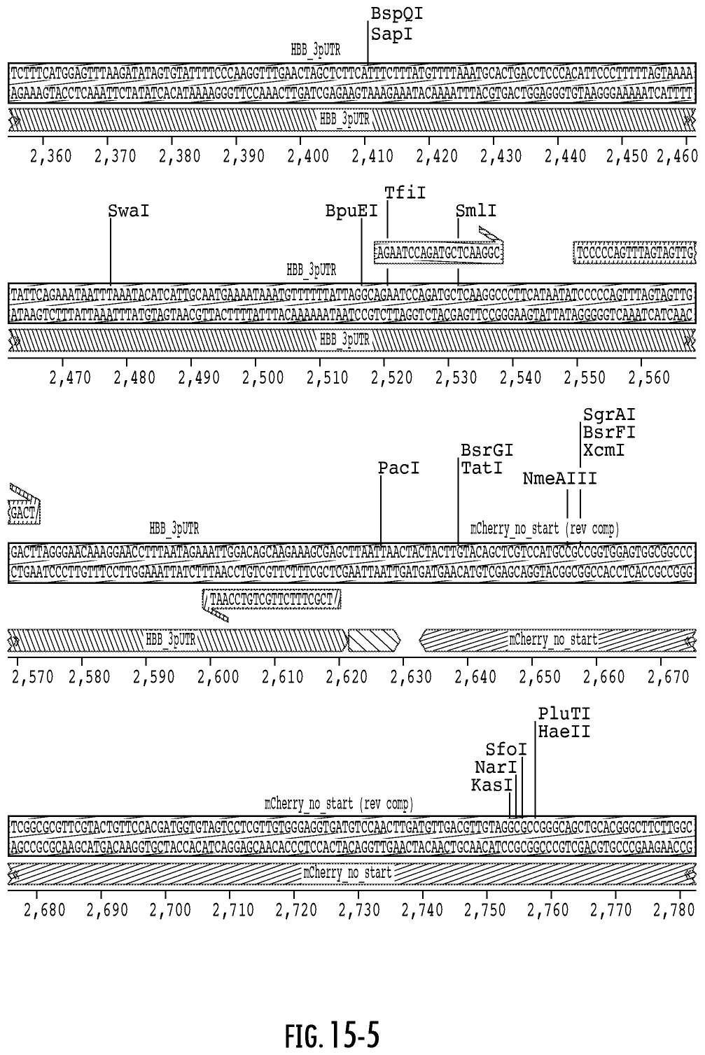

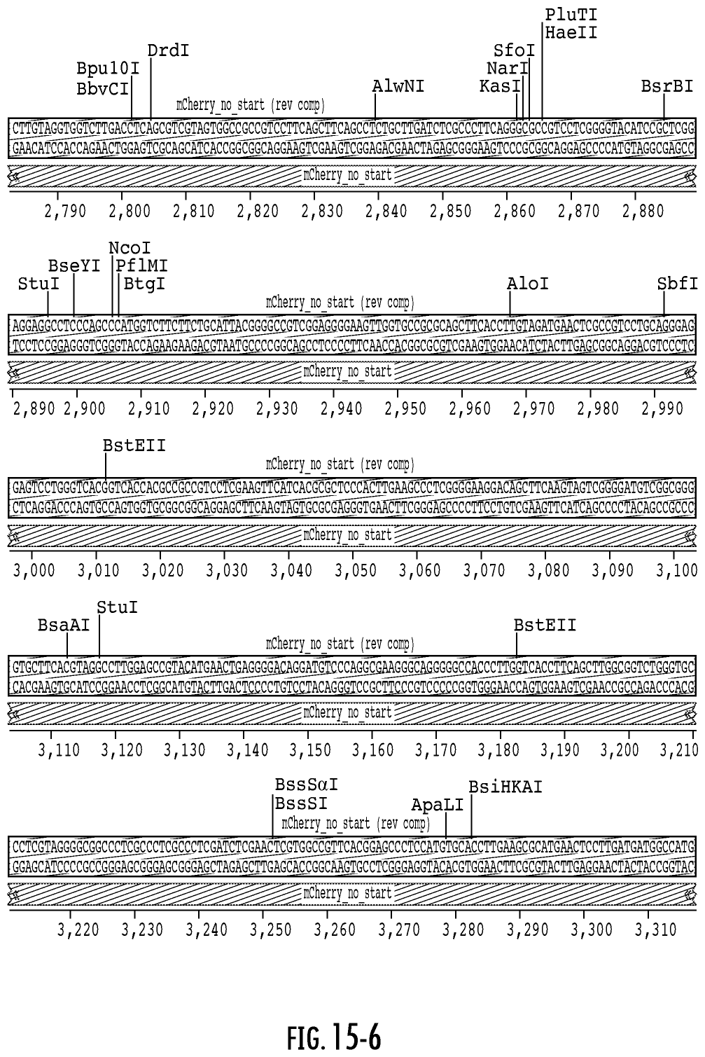

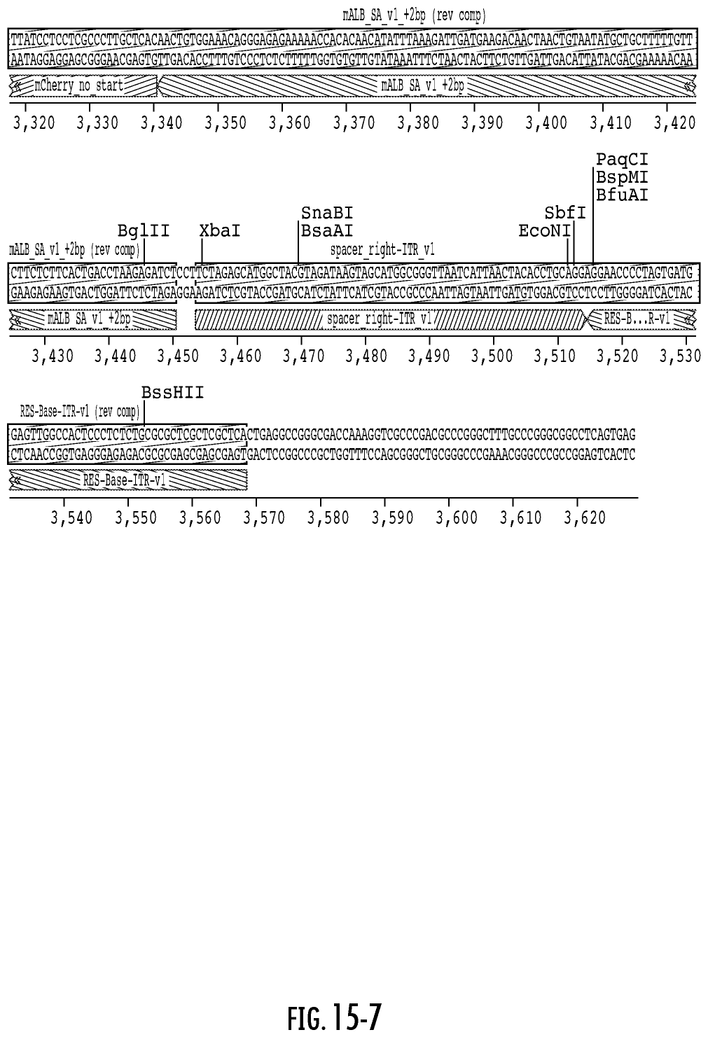

FIG. 15 shows the nucleic acid sequence of noncleavable ceDNA 346 (SEQ ID NO: 39).

FIG. 16A shows the nucleic acid sequence of cleavable ceDNA 347 (SEQ ID NO: 40). As shown in FIG. 16B, the first (SEQ ID NO: 81) and second (SEQ ID NO: 82) spacer (stuffer) sequences are bolded, and the first and second sgRNA and PAM sequences (SEQ ID NO: 83 and SEQ ID NO: 84, respectively) are bolded and underlined.

FG. 17A shows the nucleic acid sequence of cleavable ceDNA 348 (SEQ ID NO: 41). As shown in FIG. 17B, the first (SEQ ID NO: 85) and second (SEQ ID NO: 86) spacer (stuffer) sequences are bolded, and the first and second sgRNA and PAM sequences (SEQ ID NO: 87 and SEQ ID NO: 88, respectively) are bolded and underlined.

FIG. 18A shows the nucleic acid sequence of cleavable ceDNA 349 (SEQ ID NO: 42). As shown in FIG. 18B, the first (SEQ ID NO: 89) and second (SEQ ID NO: 90) spacer (stuffer) sequences are bolded, and the first and second sgRNA and PAM sequences (SEQ ID NO: 91 and SEQ ID NO: 92, respectively) are bolded and underlined.

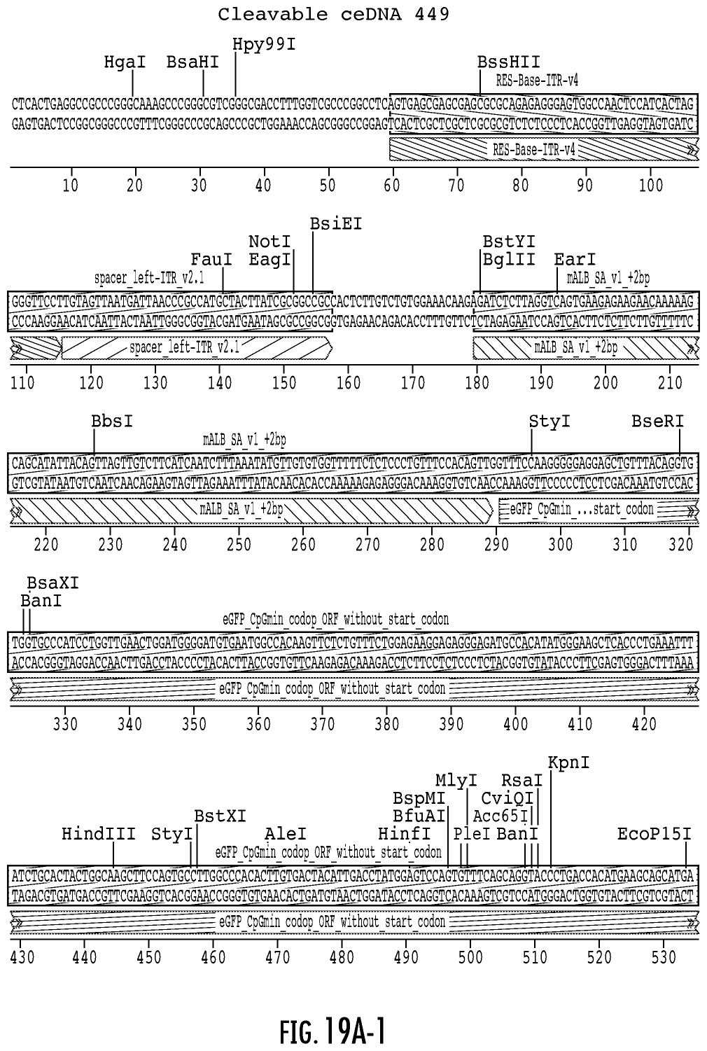

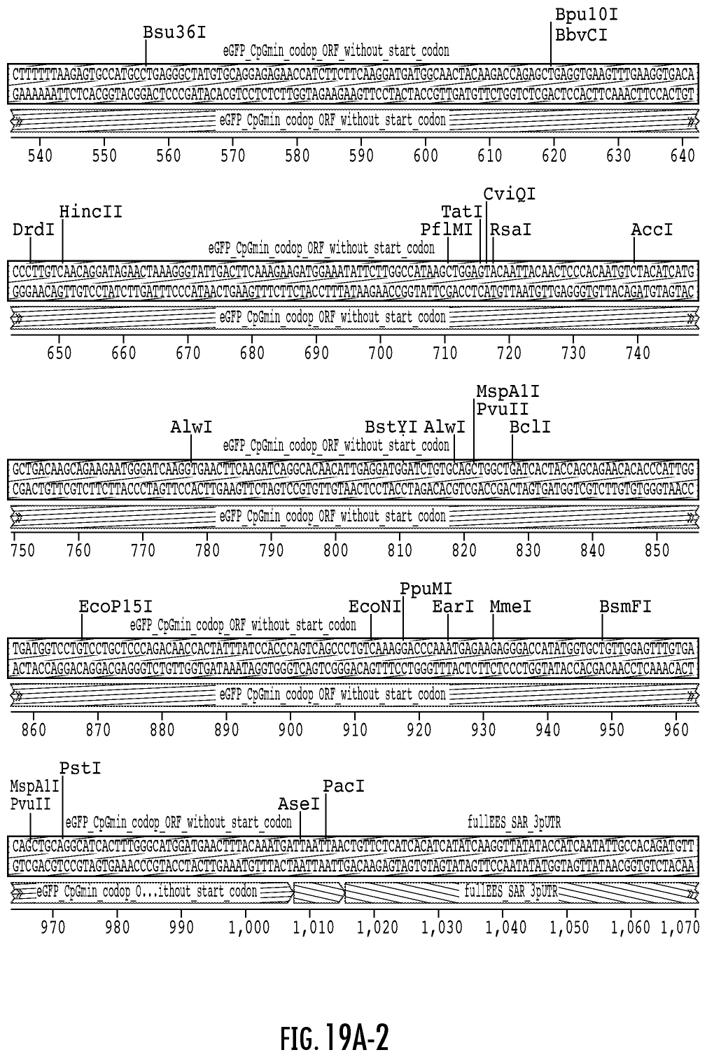

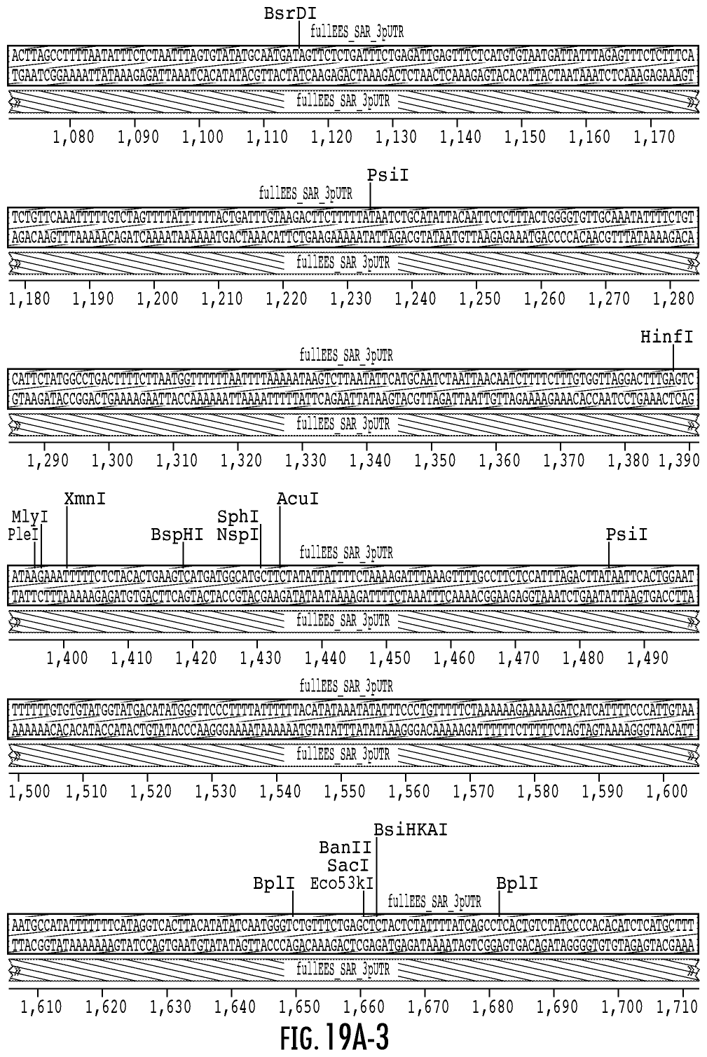

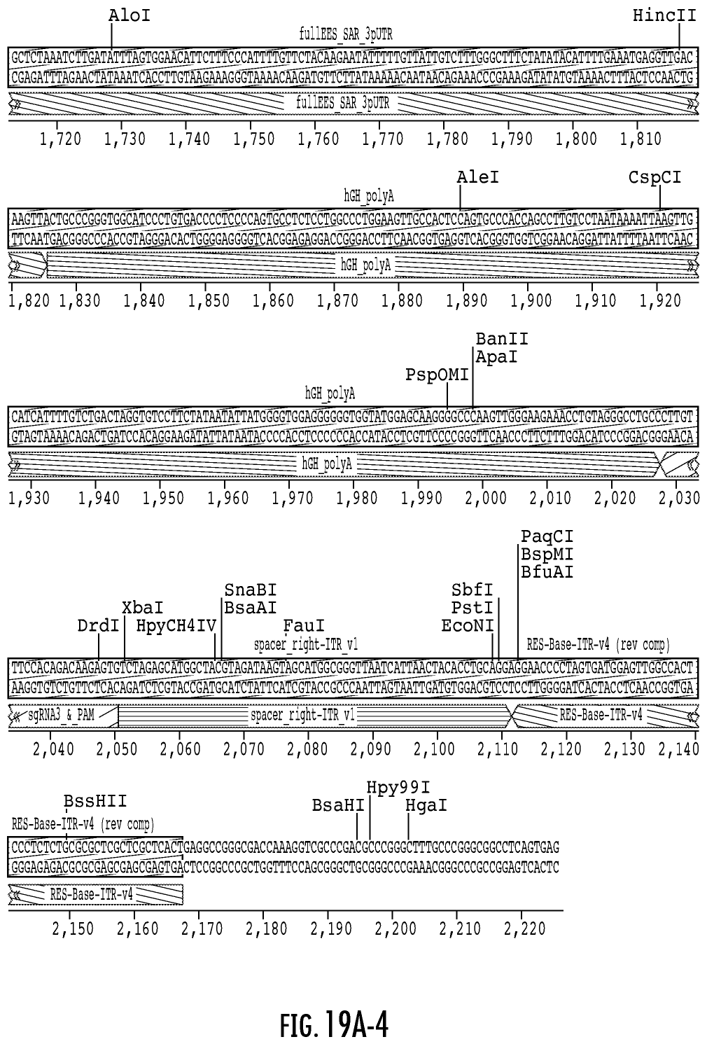

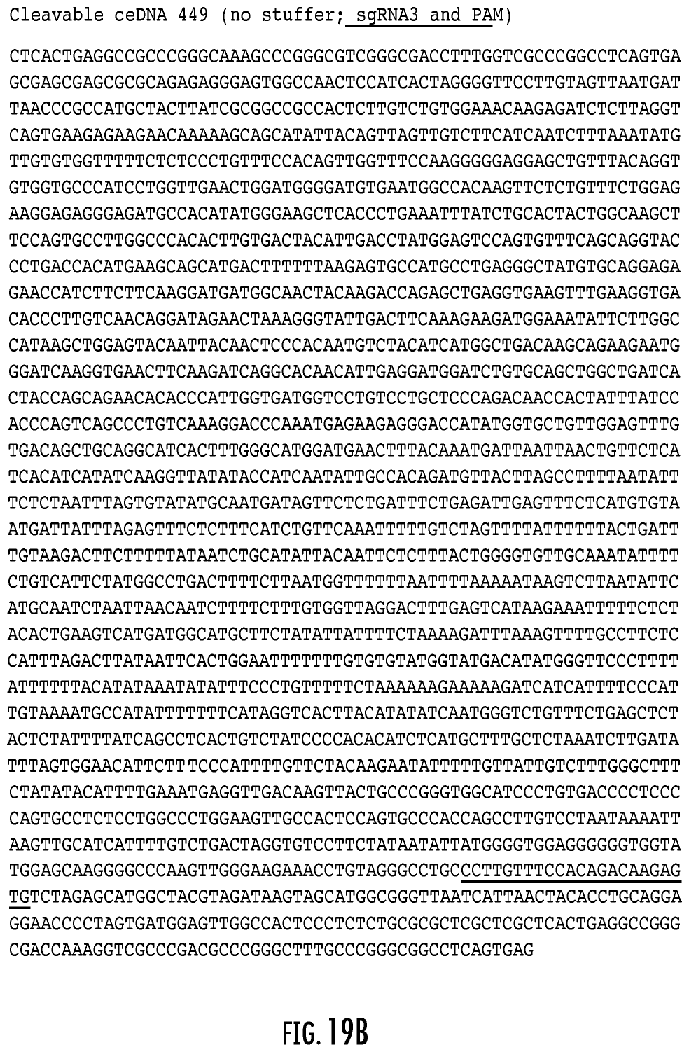

FIG. 19A shows the nucleic acid sequence of cleavable ceDNA 449 (SEQ ID NO: 43). As shown in FIG. 19B, the sgRNA and PAM sequence is bolded and underlined (SEQ ID NO: 93).

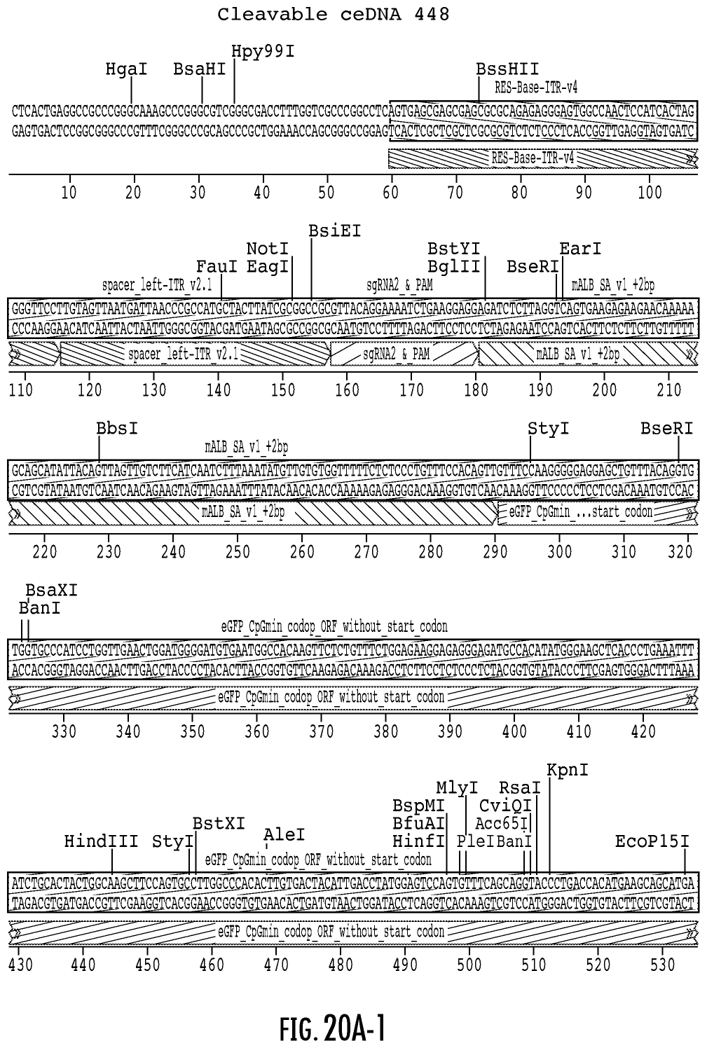

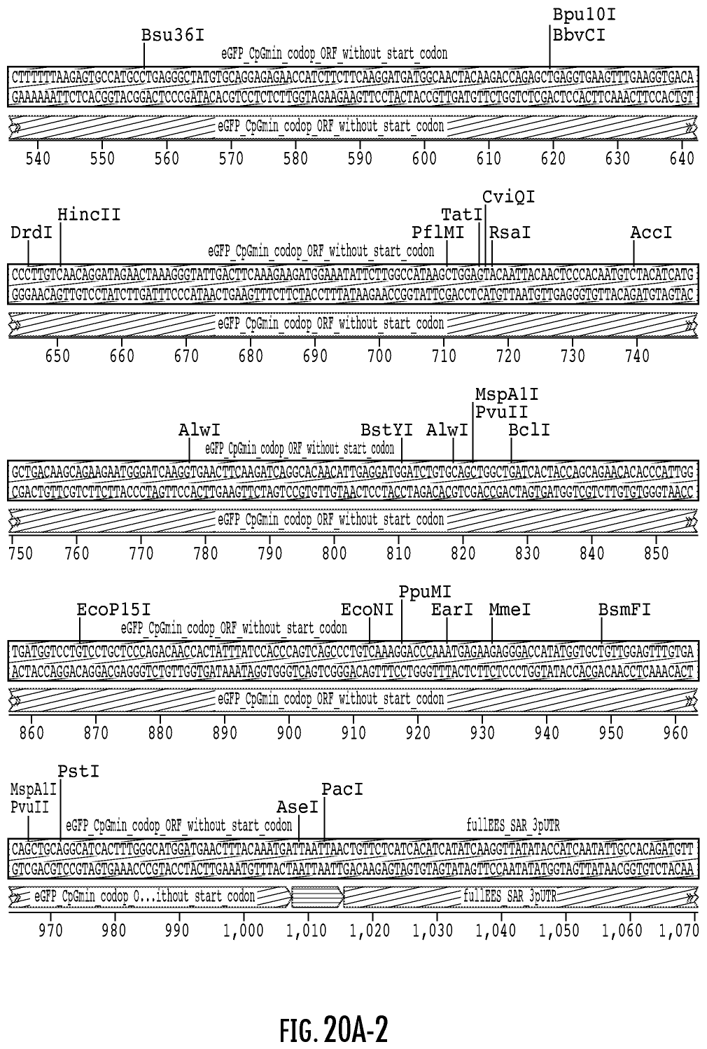

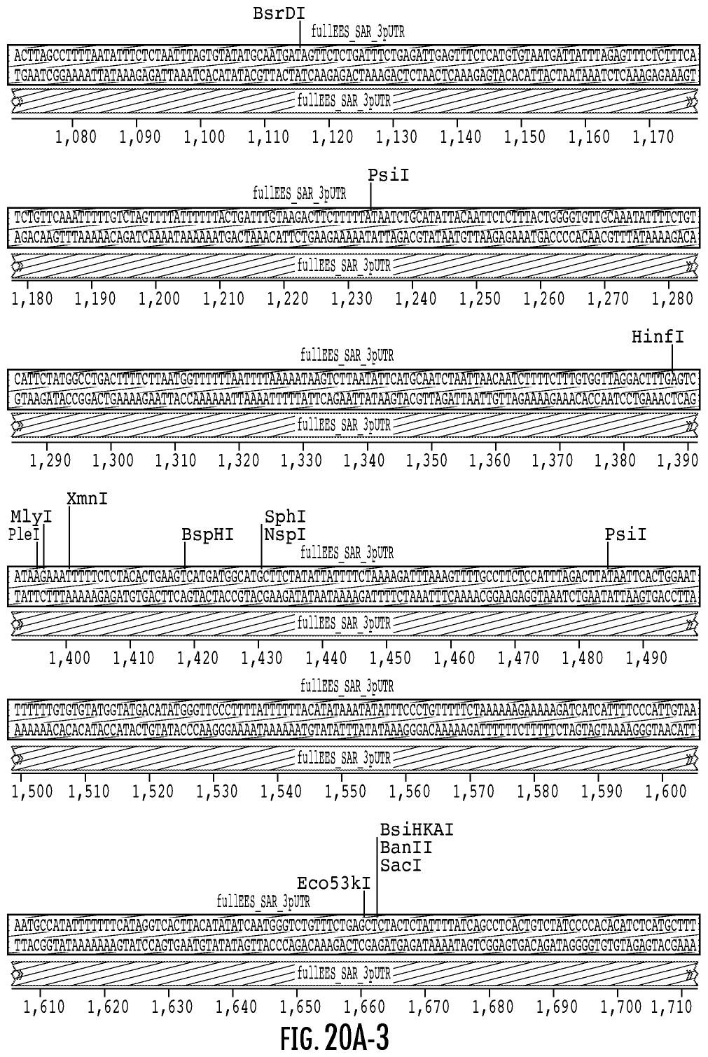

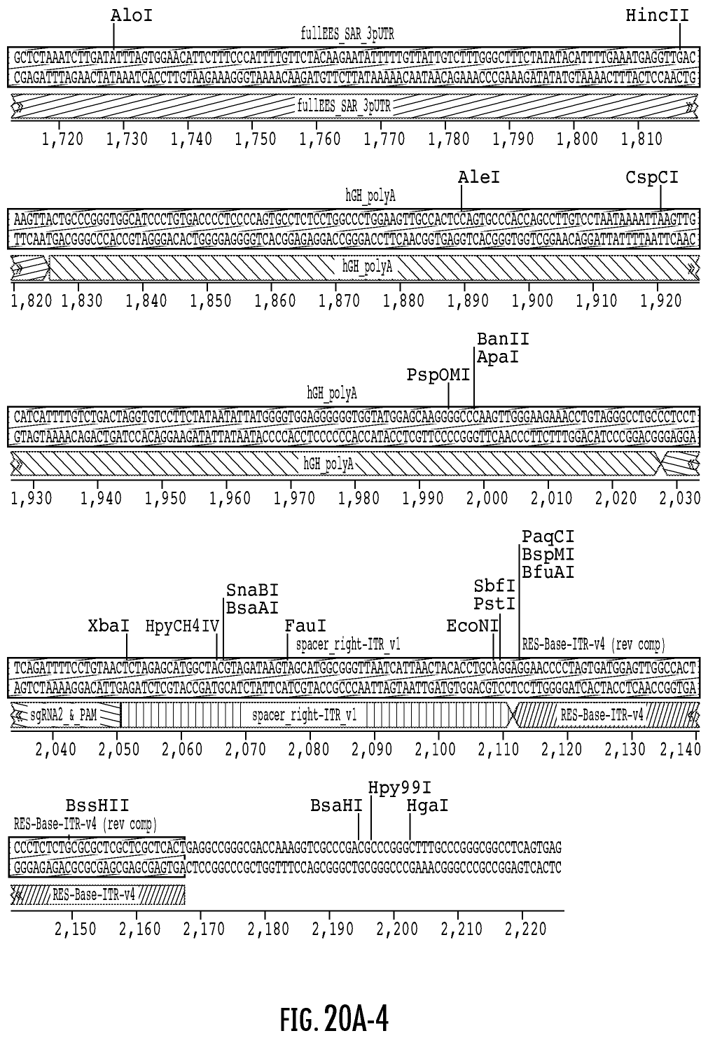

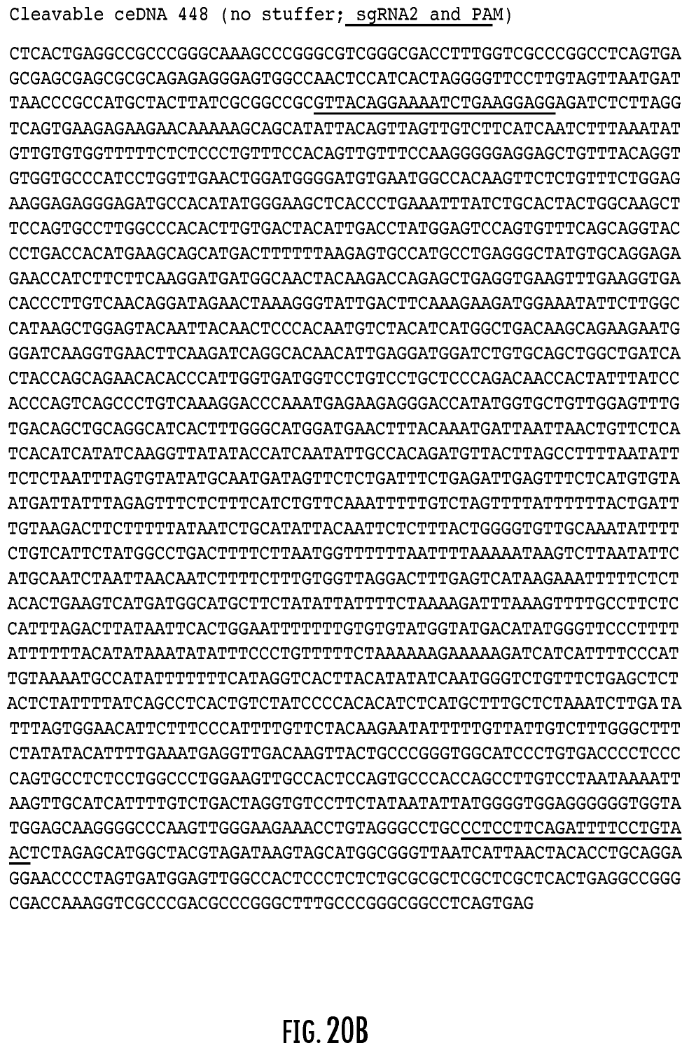

HMG. 20A shows the nucleic acid sequence of Cleavable ceDNA 448 (SEQ ID NO: 44). As shown in FIG. 20B, the first and second sgRNA and PAM sequences are bolded and underlined (SEQ ID NO: 93 and SEQ ID NO: 94, respectively).

### Example 2: Small-Scale ceDNA Production and In-Vitro Cleavage by a Site-Specific Nuclease

To confirm that cleavable ceDNA would be targeted and cleaved by a site-specific nuclease, e.g., Cas9, a biochemical experiment was performed in which standard or cleavable ceDNA was incubated with Cas9 and the appropriate sgRNA for the target sequence. These samples were then run on a bioanalyzer to quantify the percent of ceDNA that had been successfully cleaved.

In this series of experiments, ceDNA was generated from synthetic plasmids containing the mature eGFP sequence flanked by splice acceptor sites for the mouse albumin locus and sgRNA target sequences selected from the patent publication US2020/0289628A1, incorporated by reference in its entirety herein (SEQ ID NOs. 100 and 103) and either no spacer, a 50 bp spacer, or a 100 bp spacer located immediately adjacent to gRNA TS/PAM. Cell-free digestion of these constructs was then performed using purified Cas9 in order to determine the effect of spacer length on the steric hindrance of the ITRs on the nuclease activity of Cas9.

### Methods

ceDNA Generation

ceDNA was made using the cell-free protocol as described in International Patent Application Publication No. WO2019/0143885 set forth below.

Detailed Procedure for Cell-Free Synthesis of ceDNA

Lyophilized ITR oligonucleotides are re-suspended in 1×TE buffer at 100 μM concentration to create a stock, which are diluted 1:10 in Duplex Buffer (30 mM HEPES, pH 7.5; 100 mM potassium acetate) to give a final concentration of 10 μM for use. To induce self-annealing, the diluted ITR oligonucleotides are incubated at 95° C. for 10 min, which are the removed and immediately placed in an iced water bath to rapidly cool.

Next, an enzymatic combined digestion and ligation reaction mixture is set up with a double-stranded construct, a restriction endonuclease, and T4 ligase. The Synthetic Plasmid used as a backbone was AOP-900001, so the enzyme used to cleave it was Esp3l.

The reaction was scaled down to a 60 uL total volume with the following components:

| Reagent | Volume (ul) |
|---|---|
| Water † | 4.5 |
| pHK based plasmid (−200 ng/uL) | 40 |
| Annealed Oligo (10 uM)^{§} | 6 |
| T4 DNA Ligase buffer (10X)* | 6 |
| T4 DNA Ligase (2,000 U/uL) | 0.5 |
| Type IIS enzyme (20 U/uL) | 3 |

The digestion/ligation reaction mixture was incubated at 37° C. for 2.5 h, followed by a heat inactivation of the T4 ligase at 65-75° C. for 30 min. After that, the T5 exonuclease reaction that removes residual open-ended DNA fragments from the construct backbone and unligated ITR oligonucleotides and inserts at 37° C. for 1-1.5 h was set up as follows:

### 1000-μL and 40-mL Exonuclease Digestion Reaction Mixtures

| Reagent | Volume (μL) | Volume (mL) |
|---|---|---|
| Digestion/Ligation Solution | 800 | 32.0 |
| Nuclease-Free Water | 60 | 2.4 |
| Thermo Scientific FAST | 100 | 4.0 |
| DIGEST ™ Esp31 (Catalog No. | | |
| FERFD0454) | | |
| T5 Exonuclease (10 U/μL) | 40 | 1.6 |

After T5 exonuclease digestion, the reaction mixture was purified using the ZymoPURE Gigaprep Kit according to the manufacturer's instructions.

Alternatively or additionally, the reaction mixture was mixed with a diluent buffer (50 mM sodium phosphate, 50 mM EDTA, pH 7.0) and loaded onto a packed DMAE resin column for purification.

Once purified, the cleavable ceDNA from these reactions was run on the Agilent Bioanalyzer 2100 using the DNA 7500 chip to confirm size and determine purity. Results (not shown) confirmed that all reactions had been successful in producing a single species of ceDNA. The samples loaded on the Bioanalyzer were as follows:

| Sample | Construct |
|---|---|
| 1 | Non-cleaving |
| 2 | sgRNA 100, no Spacer |
| 3 | sgRNA 100, 50 bp Spacer |
| 4 - Cleavable | sgRNA 100, 100 bp Spacer |
| ceDNA 136 | |
| 5 | sgRNA 103, no Spacer |
| 6 | sgRNA 103, 50 bp Spacer |
| 7 - Cleavable | sgRNA 103, 100 bp Spacer |
| ceDNA 138 | |

### In-Vitro Cleavage Reaction:

Cas9 Nuclease, S. pyogenes, (Cas9) is a double-stranded DNA endonuclease that is guided to its target by sequence complementarity of a small RNA loaded into the protein. The following protocol was used to digest double-stranded DNA in vitro using Cas9 and a single guide RNA (sgRNA), with a reaction size of 50 uL.

### Required Materials:

Cas9 Nuclease, S. pyogenes (NEB #M0386)
NEBuffer r3.1
Nuclease-free water
Proteinase K, Molecular Biology Grade (NEB #P8107S)
sgRNA containing the targeting sequence in the region of interest
sgRNAs can be generated by in vitro transcription using the ENGEN® sgRNA Synthesis Kit, S. pyogenes (NEB/E332.) and a single oligonucleotide or the HiScribe T7 Quick High-Yield RNA synthesis Kit (NEB #E2050) using linearized plasmid, PCR products, or oligonucleotides as templates.

Duplicates of the reaction were used for each construct and the digestion was performed both for 15 minutes (as described in the protocol) and for one hour in order to evaluate the effect of increasing incubation time on cleavage efficiency. Once purified, the cleavable ceDNA from these reactions was run on the Agilent Bioanalyzer 2100 using the DNA 7500 chip to evaluate cleavage efficiency.

ceDNA Protocol:

The results of the in-vitro cleavage with Cas9 were determined using the Agilent Bioanalyzer 2100 with the ONA 7500 chip and program. The samples were organized as shown below. sgRNA 100 Constructs 15-minute Digest:

| Sample | Construct | Condition |
|---|---|---|
| 1 | Non-cleaving | Digested by Cas9 |
| 2 | sgRNA 100, no Spacer | Digested by Cas9 |
| 3 | sgRNA 100, no Spacer | Digested by Cas9 |
| 4 | sgRNA 100, 50 bp Spacer | Digested by Cas9 |
| 5 | sgRNA 100, 50 bp Spacer | Digested by Cas9 |
| 6 | sgRNA 100, 100 bp Spacer | Digested by Cas9 |
| 7 | sgRNA 100, 100 bp Spacer | Digested by Cas9 |
| 8 | Non-cleaving | Undigested |
| 9 | sgRNA 100, no Spacer | Undigested |
| 10 | sgRNA 100, 50 bp Spacer | Undigested |
| 11 | sgRNA 100, 100 bp Spacer | Undigested |
| 12 | H₂O | N/A |

| Sample | Construct | Condition |
|---|---|---|
| 1 | Non-cleaving | Digested by Cas9 |
| 2 | sgRNA 100, No Spacer | Digested by Cas9 |
| 3 | sgRNA 100, No Spacer | Digested by Cas9 |
| 4 | sgRNA 100, 50 bp Spacer | Digested by Cas9 |
| 5 | sgRNA 100, 50 bp Spacer | Digested by Cas9 |
| 6 | sgRNA 100, 100 bp Spacer | Digested by Cas9 |
| 7 | sgRNA 100, 100 bp Spacer | Digested by Cas9 |
| 8 | Non-cleaving | Undigested |
| 9 | sgRNA 100, No Spacer | Undigested |
| 10 | sgRNA 100, 50 bp Spacer | Undigested |
| 11 | sgRNA 100, 100 bp Spacer | Undigested |
| 12 | H₂O | N/A |

| Sample | Construct | Condition |
|---|---|---|
| 1 | Non-cleaving | Digested by Cas9 |
| 2 | sgRNA 103, no Spacer | Digested by Cas9 |
| 3 | sgRNA 103, no Spacer | Digested by Cas9 |
| 4 | sgRNA 103, 50 bp Spacer | Digested by Cas9 |
| 5 | sgRNA 103, 50 bp Spacer | Digested by Cas9 |
| 6 | sgRNA 103, 103 bp Spacer | Digested by Cas9 |
| 7 | sgRNA 103, 103 bp Spacer | Digested by Cas9 |
| 8 | Non-cleaving | Undigested |
| 9 | sgRNA 103, no Spacer | Undigested |
| 10 | sgRNA 103, 50 bp Spacer | Undigested |
| 11 | sgRNA 103, 103 bp Spacer | Undigested |
| 12 | H₂O | N/A |

| Sample | Construct | Condition |
|---|---|---|
| 1 | Non-cleaving | Digested by Cas9 |
| 2 | sgRNA 103, No Spacer | Digested by Cas9 |
| 3 | sgRNA 103, No Spacer | Digested by Cas9 |
| 4 | sgRNA 103, 50 bp Spacer | Digested by Cas9 |
| 5 | sgRNA 103, 50 bp Spacer | Digested by Cas9 |
| 6 | sgRNA 103, 103 bp Spacer | Digested by Cas9 |
| 7 | sgRNA 103, 103 bp Spacer | Digested by Cas9 |
| 8 | Non-cleaving | Undigested |
| 9 | sgRNA 103, No Spacer | Undigested |
| 10 | sgRNA 103, 50 bp Spacer | Undigested |
| 11 | sgRNA 103, 103 bp Spacer | Undigested |
| 12 | H₂O | N/A |

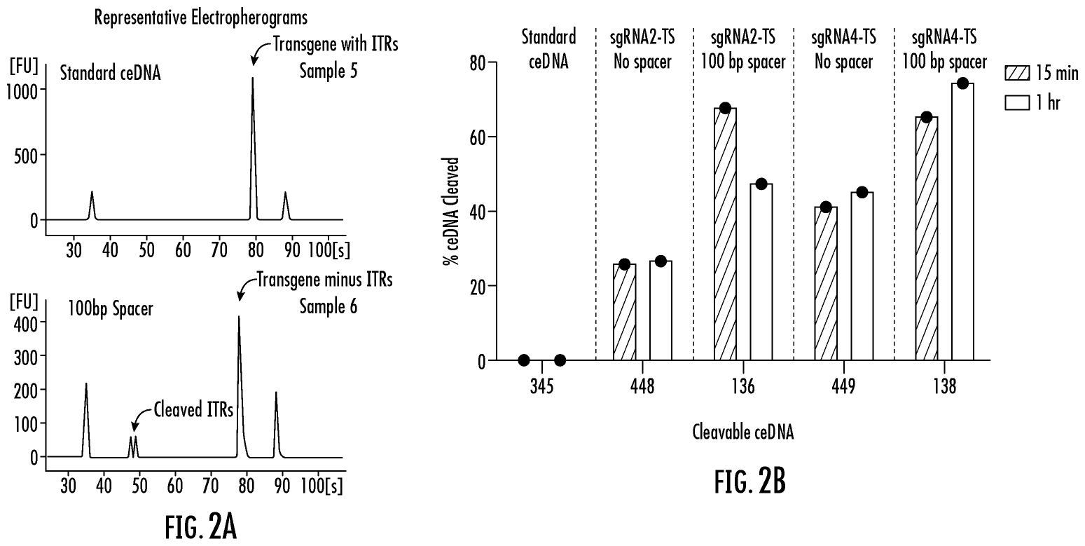

The results are shown in FIG. 2 and FIG. 3, and demonstrate that cleavable ceDNA was effectively cleaved by Cas9. The results clearly showed cleavage of the cleavable ceDNA by Cas9 for all constructs containing either no spacer or a 100 bp spacer, with higher cleavage efficiency achieved by the longer spacer and longer incubation time with Cas9. The Bioanalyzer results were also collected and evaluated quantitatively (not shown), and the results are shown in the graph in FIG. 3. As shown in the graph, this reaction was more efficient when a spacer sequence was included between the sgRNA TS+PAM and the ITRs of ceDNA, as shown by the higher percent ceDNA cleaved in cleavable ceDNA 136 and Cleavable ceDNA 138. A spacer between the ITR and the gRNA TS/PAM may reduce steric hindrance from the ITR on the site-specific nuclease activity.

### Example 3: Gene Insertion Experiments In Vitro

Gene insertion experiments will be performed in primary mouse hepatocytes. Primary mouse hepatocytes are isolated following previously described protocols (e.g., collagenase digestion), cells are tested for viability, plated, and are incubated overnight. After overnight incubation, cells are transfected with standard ceDNA or cleavable ceDNA vectors as described herein, in the presence of Cas9. Next-generation sequencing (NGS) will be performed.

### Example 4: Gene Insertion Experiments In Vivo

Gene insertion experiments will be performed to compare the efficiency of standard ceDNA versus cleavable ceDNA to serve as a donor sequence for gene insertion into the albumin locus in liver. Next-generation sequencing (NGS) will be performed.

### Example 5: Generation of a Cleavable ceDNA Vector for Treating Disease

For illustrative purposes, this Example describes generating exemplary cleavable ceDNA vectors for treating different diseases. However, while certain genes for cystic fibrosis, liver disorders, systemic disorders, CNS disorders and muscle disorders are exemplified in this Example to illustrate methods to generate a cleavable ceDNA vector useful in the methods and constructs as described herein, one of ordinary skill in the art is aware that one can modify the target gene to treat any disease where gene editing is desired. Exemplary diseases or genetic disorders where gene editing is a desired strategy to treat a disease with a cleavable ceDNA editing vector as described herein is discussed in the sections entitled “Exemplary diseases to be treated” and “additional diseases.”

In one example, a cleavable ceDNA vector can be generated according to Example 1.

An exemplary transgene replacing or providing cleavable ceDNA vector can be configured to induce gene editing with distinct transgenes for other genetic disorders, including liver disorders (e.g., OTC, GSD I a, Crigler-Najar, PKU, and the like) or systemic disorders (e.g., MPSII, MLD, MPSIIIA, Gaucher, Fabry, Pompe, and the like).

In one example, a cleavable ceDNA vector is created to encode CFTR, the gene that is mutated in CF. CFTR is a large gene that cannot be comprised within AAV. Therefore, a cleavable ceDNA vector provides a unique solution and can, in some embodiments, be administered intravenously and/or as a nebulized formulation to a subject to induce gene editing of lung epithelia. As above, a cleavable ceDNA vector is configured such that CFTR is inserted into the endogenous CFTR locus. In such an example, a composition comprising the cleavable ceDNA vector can also comprise a nuclease and at least one guide RNA as well as, utilizing large homology arms to increase the efficiency and fidelity of gene editing.

In another example, a cleavable ceDNA is designed to induce gene editing in the CNS, for disorders including neurodegenerative disorders (e.g., familial forms of Alzheimer's, Parkinson's, Huntington's), lysosomal storage disorders (e.g., MPSII, MLD, MPSIIIA, Canavan, Batten, and the like) or neurodevelopmental disorders (e.g., SMA, Rett syndrome, and the like)

In another example, a cleavable ceDNA vector can be designed to induce gene editing in the muscle, for disorders including but not limited to Duchenne muscular dystrophy, facioscapulohumeral dystrophy, and the like.

A cleavable ceDNA as described herein can be delivered to target cells in an animal model for the defective transgene to assess the efficacy of the gene editing and also to provide cells that produce more effective gene product.

TABLE 9
Exemplary genes for targeting (see also, e.g., US 2015/0056705,
which is incorporated herein in its entirety by reference)

| | | Representative |
|---|---|---|
| | | Accession |
| Gene name | location | (cDNA) RefSeq |
| HBB | chr11: 5246696-5248301 | (NM_000518) |
| BCL11A | chr2: 60684329-60780633 | (NM_022893) |
| KLF1 | chr19: 12995237-12998017 | (NM_006563) |
| HBG1 | chr11: 5269502-5271087 | (NM_000559) |
| CCR5 | chr3: 46411633-46417697 | (NM_000579) |
| CXCR4 | chr2: 136871919-136873813 | (NM_001008540) |
| PPP1R12C | chr19: 55602281-55628968 | (NM_017607) |
| HPRT | chrX: 133594175-133634698 | (NM_000194) |
| Mouse HPRT | chrX: 52988078-53021660 | (NM_013556) |
| | (assembly GRCm38/mm10) | |
| ALB | chr4: 74269972-74287129 | (NM_000477) |
| Factor VIII | chrX: 154064064-154250998 | (NM_000132.3) |
| Factor IX | chrX: 138612895-138645617 | (NM_000133) |
| LRRK2 | chr12: 40618813-40763086 | (NM_198578) |
| Htt | chr4: 3076237-3245687 | (NM_002111) |
| RHO | chr3: 129247482-129254187 | (NM_000539) |
| CFTR | chr7: 117120017-117308718 | (NM_000492) |
| TCRA | chr6: 42883727-42893575 | (NM_001243168) |
| TCRB | chr7: 142197572-142198055 | L36092.2 |
| PD-1 | chr2: 242792033-242795132 | (NM_005018) |
| CTLA-4 | chr2: 204732511-204738683 | (NM_001037631) |
| HLA-A | chr6: 29910247-29912868 | (NM_002116) |
| HLA-B | chr6: 31236526-31239913 | NM_005514.6 |
| HLA-C | chr6: 31236526-31239125 | (NM_001243042) |
| HLA-DPA | chr6: 33032346-33048555 | (NM_033554.3) |
| HLA-DQ | chr6: 32605183-32611429 | (NM_002122) |
| HLA-DRA | chr6_ssto_hap7: 3754283- | (NM_019111) |
| | 3759493 | |
| LMP7 | chr6_dbb_hap3: 4089872- | (X66401) |
| | 4093057 | |
| Tapasin | chr6: 33271410-33282164 | (NM_172208) |
| RFX5 | chr1: 151313116-151319769 | (NM_001025603) |
| CIITA | chr16: 10971055-11002744 | (NM_000246) |
| TAP1 | chr6: 32812986-32821748 | (NM_000593) |
| TAP2 | chr6: 32793187-32806547 | (NM_000544) |
| TAPBP | chr6: 33267472-33282164 | |
| DMD | chrX: 31137345-33229673 | (NM_004006) |
| RFX5 | chr1: 151313116-151319769 | (NM_000449) |
| B. napus FAD3 | See PCT publication | JN992612 |
| | WO2014/039684 | |
| B. napus FAD2 | See PCT publication | JN992609 |
| | WO2014/039692 | |
| Soybean FAD2 | See US20140090116 | |
| Zea mays ZP15 | See U.S. Pat. No. 8,329,986 | GBWI-61522 |
| | | (MaizeCyc) |
| B-ketoacyl ACP | See U.S. Pat. No. 8,592,645 | |
| synthase II | | |
| (KASII) | | |
| Tomato MDH | See US 20130326725 | AY725474 |
| B. napus EPSPS | See U.S. Pat. No. 8,399,218 | |
| paralogs C + D | | |
| Paralog D | See U.S. Pat. No. 8,399,218 | |
| Paralog A + B | See U.S. Pat. No. 8,399,218 | |
| PPP1R12C | chr19: 55602840-55624858 | (NM_017607) |
| (AAVS1) | | |
| GR | 5: 142646254-142783254 | (NM_000176) |
| IL2RG | chrX: 70327254-70331481 | (NM_000206) |
| SFTPB | chr2: 85884440-85895374 | (NM_198843) |

### Example 6: Cleavable ceDNA for Engineering of T Cells

The cleavable ceDNA vectors described herein can be used to edit, repair, and/or knock-out genes in the genome of any cell, for example, in a T cell. For illustrative purposes, an exemplary cleavable ceDNA vector is described with respect to generating a cleaved ceDNA vector for editing any of CXCR4, CCR5, PD-1 genes in T-cells and is described below. However, while targeting CXCR4, CCR5 or PD-1 genes are exemplified in this Example to illustrate methods to generate a ceDNA useful in the methods and constructs as described herein, one of ordinary skill in the art is aware that one can use any gene where gene editing is desired, for example, as described herein in the sections entitled “Exemplary diseases to be treated” and “additional diseases”. Additionally, while the genome of T cells is modified in this illustrative example, one of ordinary skill is aware that any cell can be modified, ex vivo or in vivo, for example, any cell as described herein entitled “host cells”. Also, while genomic DNA is shown in this illustrative example to be modified, it is envisioned that the cleavable ceDNA vectors can also be modified by an ordinary skilled artisan to modify mitochondrial DNA (mtDNA), e.g., to encode mtZFN and mitoTALEN function, or mitochondrial-adapted CRISPR/Cas9 platform as described in Maeder, et al. “Genome-editing technologies for gene and cell therapy.” Molecular Therapy 24.3 (2016): 430-446 and Gammage P A, et al. Mitochondrial Genome Engineering: The Revolution May Not Be CRISPR-lzed. Trends Genet. 2018; 34(2):101-110.

Any therapeutically relevant gene can be targeted (e.g., CXCR4, or CCRS, the coreceptor for HIV entry), and can be ablated, edited, repaired or replaced (in the case of CXCR4 e.g., to prevent HIV entry). In a further non-limiting example, PD-1, a mediator of T cell exhaustion, can be ablated. Ablation of target genes is performed with or without a template nucleic acid sequence, e.g., donor template. Use of a single guide RNA (sgRNA) and corresponding nuclease in the absence of homology arms results in non-homologous-end-joining (NHEJ).

Any therapeutically-relevant locus can be targeted, such targeted loci are, e.g., known regulators of T cell exhaustion, viral coreceptors, and the like.

Further, because of ceDNA's lack of size restriction, a ceDNA vector can be engineered to provide the donor DNA, i.e., transgene cassette, and/or gene editing molecules on the same ceDNA vector. Alternatively, the donor DNA and/or gene editing molecules can be provided on one or more different ceDNA vectors.

In some experiments, the guide RNAs will target other known sequence regions. Multiple sgRNA sequences that bind known target regions are described in Tables 1-2 of US patent publication 2015/0056705, which is herein incorporated by reference in its entirety, and include for example gRNA sequences for human beta-globin, human, BCLIIA, human KLF1, Human CCR5, Human CXCR4, PPP1R12C, mouse and human HPRT, human albumin, human factor IX, human factor VIII, human LRRK2, human Htt, human RH, CFTR, TRAC, TRBC, human PD1, human CTLA-4, HLA c11, HLA A2, HLA A3, HLA B, HLA C, HLA c1, II DBp2, DRA, Tap 1 and 2, Tapasin, DMD, RFX5, etc.).

The cleavable ceDNA vectors will be delivered to T cells ex vivo, but systemic delivery is also contemplated herein.

### REFERENCES

All publications and references, including but not limited to patents and patent applications, cited in this specification and Examples herein are incorporated by reference in their entirety as if each individual publication or reference were specifically and individually indicated to be incorporated by reference herein as being fully set forth. Any patent application to which this application claims priority is also incorporated by reference herein in the manner described above for publications and references.

1. A cleavable non-viral capsid-free closed-ended DNA (ceDNA) comprising:
a first inverted terminal repeat (ITR);
a first guide RNA (gRNA) target sequence (TS) and a first protospacer adjacent motif (PAM);
at least one transgene cassette;
a second gRNA target sequence (TS) and a second protospacer adjacent motif (PAM); and
a second ITR.

2. The cleavable ceDNA of claim 1, further comprising a first spacer sequence between the first ITR and the first gRNA TS, and/or a first spacer sequence between the first PAM and the at least one transgene cassette.

3. The cleavable ceDNA of claim 2, wherein the first spacer sequence is between 1-200 nucleotides in length.

4. The cleavable ceDNA of claim 2 or claim 3, wherein the first spacer sequence is 10, 25, 50, 75, 100, 125, 150, 175, or 200 nucleotides in length.

5. The cleavable ceDNA of any one of claims 2-4, wherein the first spacer sequence has at least 85% identity to a spacer sequence selected from SEQ ID NO: 45, as shown in FIG. 3B, SEQ ID NO: 49, as shown in FIG. 4B, SEQ ID NO: 53 as shown in FIG. 5B, SEQ ID NO: 57, as shown in FIG. 6B, SEQ ID NO: 61, as shown in FIG. 8B, SEQ ID NO: 65 as shown in FIG. 9B, SEQ ID NO: 69, as shown in FIG. 10B, SEQ ID NO: 73 as shown in FIG. 11B, SEQ ID NO: 77, as shown in FIG. 14B, SEQ ID NO: 81, as shown in FIG. 16B, SEQ ID NO: 85 as shown in FIG. 17B, and SEQ ID NO: 89, as shown in FIG. 18B.

6. The cleavable ceDNA of any one of claims 2-5, further comprising a second spacer sequence between the second gRNA TS and the second ITR, and/or a second spacer sequence between the at least one transgene cassette and the second PAM.

7. The cleavable ceDNA of claim 6, wherein the second spacer sequence is between 1-200 nucleotides in length.

8. The cleavable ceDNA of claim 6 or claim 7, wherein the second spacer sequence is 10, 25, 50, 75, 100, 125, 150, 175, or 200 nucleotides in length.

9. The cleavable ceDNA of any one of claims 6-8, wherein the second spacer sequence has at least 85% identity to a spacer sequence selected from SEQ ID NO: 46, as shown in FIG. 3B, SEQ ID NO: 50, as shown in FIG. 4B, SEQ ID NO: 54 as shown in FIG. 5B, SEQ ID NO: 58, as shown in FIG. 6B, SEQ ID NO: 62, as shown in FIG. 8B, SEQ ID NO: 66 as shown in FIG. 9B, SEQ ID NO: 70, as shown in FIG. 10B, SEQ ID NO: 74 as shown in FIG. 11B, SEQ ID NO: 78, as shown in FIG. 14B, SEQ ID NO: 82, as shown in FIG. 16B, SEQ ID NO: 86 as shown in FIG. 17B, and SEQ ID NO: 90, as shown in FIG. 18B.

10. The cleavable ceDNA of any one of claims 6-9, wherein the first spacer sequence and the second spacer sequence are the same spacer sequence or different spacer sequences.

11. The cleavable ceDNA of any one of the previous claims, wherein the first PAM comprises a nucleic acid sequence selected from the group consisting of: 5′-NGG-3′, 5′-NGAAA-3′, 5′-NNG-3′, 5′-NGA-3′, 5′-NTAA-3′, 5′-NTG-3′, 5′-NNC-3′, 5′-NNAAC-3′, 5′-AGA-3′, 5′-NNNANNA-3′, 5′-NNANAA-3′, 5′-NNAAAA-3′, and 5′-AAAA-3′; and/or wherein the second PAM comprises a nucleic acid sequence selected from the group consisting of: 5′-NGG-3′, 5′-NGAAA-3′, 5′-NNG-3′, 5′-NGA-3′, 5′-NTAA-3′, 5′-NTG-3′, 5′-NNC-3′, 5′-NNAAC-3′, 5′-AGA-3′, 5′-NNNANNA-3′, 5′-NNANAA-3′, 5′-NNAAAA-3′, and 5′-AAAA-3′.

12. The cleavable ceDNA of any one of the previous claims, wherein the first PAM and the second PAM comprise a nucleic acid sequence that is the same PAM sequence or a different PAM sequence.

13. The cleavable ceDNA of any one of the previous claims, wherein the first or second PAM is 3′ to the first gRNA TS.

14. The cleavable ceDNA of any one of the previous claims, wherein the first gRNA TS comprises a nucleic acid sequence that is the same as a nucleic acid sequence of the second gRNA TS.

15. The cleavable ceDNA of any one of the previous claims, wherein the first gRNA TS comprises a nucleic acid sequence that is different than a nucleic acid sequence of the second gRNA TS.

16. The cleavable ceDNA of any one of the previous claims, further comprising a third gRNA target sequence (TS) and a third protospacer adjacent motif (PAM).

17. The cleavable ceDNA of claim 16, wherein the third gRNA target sequence (TS) and a third protospacer adjacent motif (PAM) are located within the at least one transgene cassette.

18. The cleavable ceDNA of any one of claims 16-17, further comprising a fourth gRNA target sequence (TS) and a fourth protospacer adjacent motif (PAM).

19. The cleavable ceDNA of any one of the previous claims, wherein the at least one transgene cassette comprises a 5′ homology arm, a donor sequence, and a 3′ homology arm.

20. The cleavable ceDNA of claim 18, wherein the 5′ homology arm and the 3′ homology arm are each between about 10 to 2000 bp in length.

21. The cleavable ceDNA of claim 18 or claim 19, wherein the 5′ homology arm and the 3′ homology arm are each between about 1000 to 2000 bp in length.

22. The cleavable ceDNA of claim 18, wherein the 5′ homology arm and the 3′ homology arm are each between about 2 to 1000 bp in length.

23. The cleavable ceDNA of any one of claims 19-22, wherein the 5′ homology arm comprises a splice donor site for a target locus, and wherein the 3′ homology arm comprises a splice acceptor site for a target locus.

24. The cleavable ceDNA of any one of the previous claims, wherein the at least one transgene cassette is capable of effecting homology directed recombination (HDR), or microhomology-mediated end joining (MMFJ) and editing of a sequence at a target locus.

25. The cleavable ceDNA of any one of the previous claims, wherein the at least one transgene cassette comprises a donor sequence and does not comprise a 5′ homology arm and a 3′homology arm.

26. The cleavable ceDNA of any one of claims 19-25, wherein the donor sequence comprises a splice donor site and/or a splice acceptor site for a target locus.

27. The cleavable ceDNA of claim 25, wherein the at least one transgene cassette is capable of effecting homology directed recombination (HDR), or microhomology-mediated end joining (MMEJ) and editing of a sequence at a target locus.

28. The cleavable ceDNA of any one of the previous claims, wherein the at least one transgene cassette comprises all or a fragment of: an exon of a target gene, an intron of a target gene, a promoter region of a target gene, an enhancer region of a target gene, and/or a transcribed region of a target gene.

29. The cleavable ceDNA of claim 28, wherein the target gene is selected from a gene listed in Table 9.

30. The cleavable ceDNA of any one of the previous claims, wherein the first gRNA TS and the second gRNA TS are each a single guide RNA (sgRNA) target sequence.

31. The cleavable ceDNA of any one of claims 17-30, wherein the third gRNA TS comprises a nucleic acid sequence that is the same as a nucleic acid sequence of the first gRNA TS and/or second gRNA TS.

32. The cleavable ceDNA of any one of claims 17-31, wherein the third PAM comprises a sequence that is the same as the first PAM and/or the second PAM.

33. The cleavable ceDNA of any one of claims 17-31, wherein the at least one transgene cassette further comprises a fourth gRNA TS and a fourth PAM.

34. The cleavable ceDNA of claim 33, wherein the fourth gRNA TS comprises a nucleic acid sequence that is the same as a nucleic acid sequence of the first gRNA TS, the second gRNA TS, and/or the third gRNA TS.

35. The cleavable ceDNA of claim 33 or claim 34, wherein the fourth PAM comprises a sequence that is the same as the first PAM, the second PAM, and/or the third PAM.

36. The cleavable ceDNA of any one of the previous claims, comprising two transgene cassettes, three transgene cassettes, four transgene cassettes, or five transgene cassettes.

37. The cleavable ceDNA of claim 36, wherein the transgene cassettes are unidirectional or bidirectional.

38. The cleavable ceDNA of claim 36 or claim 37, comprising at least a third gRNA TS and a third PAM between a first transgene cassette and a second transgene cassette.

39. The cleavable ceDNA of any one of the previous claims, wherein the first ITR comprises a functional terminal resolution site and a Rep binding site, and/or wherein the second ITR comprises a functional terminal resolution site and a Rep binding site.

40. The cleavable ceDNA of any one of the previous claims, wherein the first ITR and the second ITR are symmetric or asymmetric.

41. The cleavable ceDNA of any one of the previous claims, wherein the first ITR and the second ITR are asymmetric, and wherein at least one of the ITRs is altered from a wild-type AAV ITR sequence by a deletion, addition, or substitution that affects the overall three-dimensional conformation of the ITR.

42. The cleavable ceDNA of any one of the previous claims, wherein the first ITR and/or the second ITR are derived from an AAV serotype selected from AAV1, AAV2, AAV3, AAV4, AAV5, AAV6, AAV7, AAV8, AAV9, AAV10, AAV11, and AAV12.

43. The cleavable ceDNA of any one of the previous claims, wherein the first ITR and/or the second ITR is synthetic.

44. The cleavable ceDNA of any one of the previous claims, wherein the first ITR and/or the second ITR is not a wild type ITR.

45. The cleavable ceDNA of any one of the previous claims, wherein the first ITR and/or the second ITR is modified by a deletion, insertion, and/or substitution in at least one of the ITR regions selected from A, A′, B, B′, C, C′, D, and D′.

46. The cleavable ceDNA of claim 45, wherein the first ITR and/or the second ITR is modified by a deletion of all or part of a stem-loop structure normally formed by the A, A′, B, B′ C, C′, D, and D′ regions.

47. The cleavable ceDNA any one of claims 1-40 or 42-46, wherein the first ITR and the second ITR are symmetric.

48. The cleavable ceDNA of any one of the previous claims, wherein the first ITR and/or the second ITR are wild type.

49. An isolated polynucleotide comprising the cleavable ceDNA of any one of the previous claims.

50. A gene editing system comprising:
the cleavable ceDNA of any one of claims 1-48 or the isolated polynucleotide of claim 49;
at least one guide RNA (gRNA); and
at least one site-specific nuclease enzyme or a messenger ribonucleic acid (mRNA) encoding the at least one site-specific nuclease enzyme.

51. The gene editing system of claim 50, wherein the at least one gRNA is capable of binding to both the first gRNA TS and the second gRNA TS.

52. The gene editing system of claim 50 or 51, wherein the at least one gRNA is a single guide RNA (sgRNA).

53. The gene editing system of any one of claims 50-52,
wherein the at least one gRNA is further capable of binding to a first genomic gRNA TS in a genome of a host cell, and/or
wherein the at least one gRNA is further capable of binding to both a first genomic gRNA TS in a genome of a host cell and a second genomic gRNA TS in the genome of the host cell.

54. The gene editing system of any one of claims 50-53, further comprising at least a second gRNA.

55. The gene editing system of claim 54, wherein the at least one gRNA is capable of binding to the first gRNA TS, and wherein the second gRNA is capable of binding to the second gRNA TS.

56. The gene editing system of claim 54 or claim 55, wherein the at least one gRNA is further capable of binding to a first genomic gRNA TS in a genome of a host cell, and wherein the second gRNA is further capable of binding to a second genomic gRNA TS in the genome of the host cell.

57. The gene editing system of any one of claims 50-56, further comprising a second gRNA and at least a third gRNA.

58. The gene editing system of claim 57,
wherein the at least one gRNA is capable of binding to the first gRNA TS, the second gRNA is capable of binding to the second gRNA TS, and the third gRNA is capable of binding to a first genomic gRNA TS in a genome of a host cell; or
wherein the at least one gRNA is capable of binding to the first gRNA TS and the second gRNA TS, and the second gRNA is capable of binding to a first genomic gRNA TS in a genome of a host cell, and the third gRNA is capable of binding to a second genomic gRNA TS in the genome of the host cell.

59. The gene editing system of any one of claims 50-57, further comprising a second gRNA, a third gRNA, and at least a fourth gRNA.

60. The genome editing system of claim 59, wherein the at least one gRNA is capable of binding to the first gRNA TS, the second gRNA is capable of binding to the second gRNA TS, the third gRNA is capable of binding to a first genomic gRNA TS in a genome of a host cell, and the at least fourth gRNA is capable of binding to a second genomic gRNA TS in the genome of the host cell.

61. The gene editing system of any one of claims 50-60, wherein the site-specific nuclease is a Cas9 enzyme.

62. The genome editing system of claim 61, wherein the Cas9 enzyme is a wild-type Cas9 protein, a nicking Cas9 protein (nCas9) or a dead Cas9 protein (dCas9).

63. The gene editing system of claim 61 or 62, wherein the nCas9 contains a mutation in the HNH or RuVc domain of Cas.

64. The gene editing system of any one of claims 61-63, wherein the Cas9 enzyme is a S. pyogenes Cas9.

65. The gene editing system of any one of claims 50-64, wherein the cleavable ceDNA is formulated as a lipid nanoparticle composition.

66. The gene editing system of any one of claims 50-65, wherein the at least one gRNA and the at least one site-specific nuclease enzyme or a messenger ribonucleic acid (mRNA) encoding the at least one site-specific nuclease enzyme are formulated as a lipid nanoparticle composition.

67. The gene editing system of claim 65 or claim 66, wherein the cleavable ceDNA is formulated as a first lipid nanoparticle composition and the at least one gRNA, and the at least one site-specific nuclease enzyme or a messenger ribonucleic acid (mRNA) encoding the at least one site-specific nuclease enzyme are formulated as a second lipid nanoparticle composition.

68. A lipid nanoparticle composition comprising the gene editing system of any one of claims 50-64.

69. A lipid nanoparticle composition comprising the cleavable ceDNA of any one of claims 1-48 or the isolated polynucleotide of claim 49.

70. A cell comprising the cleavable ceDNA of any one of claims 1-48, the isolated polynucleotide of claim 49, the gene editing system of any one of claims 50-67, or the lipid nanoparticle of claim 68 or claim 69.

71. A method of editing a target nucleic acid sequence in a genome of a cell, the method comprising contacting the cell with the gene editing system of any one of claims 50-67 or the lipid nanoparticle of claim 68 or 69, thereby editing the target nucleic acid in the genome of the cell.

72. The method of claim 71,
wherein the at least one gRNA and the site-specific nuclease enzyme co-localize to at least the first gRNA TS of the cleavable ceDNA and the second gRNA TS of the cleavable ceDNA, and the site-specific nuclease enzyme cleaves the cleavable ceDNA in a site-specific manner; and
wherein the at least one gRNA and the site-specific nuclease enzyme co-localize to at least a first genomic gRNA TS and/or a second genomic gRNA TS at or near the target nucleic acid in the genome of the cell, and the site-specific nuclease enzyme cleaves the genome of the cell in a site-specific manner.

73. The method of claim 71, wherein at least one gRNA and a second gRNA are present,
wherein the at least one gRNA and the site-specific nuclease enzyme co-localize to the first gRNA TS of the cleavable ceDNA, the second gRNA and the site-specific nuclease enzyme co-localize to the second gRNA TS of the cleavable ceDNA, and the site-specific nuclease enzyme cleaves the cleavable ceDNA in a site-specific manner; and
wherein the at least one gRNA and the site-specific nuclease enzyme co-localize to a first genomic gRNA TS at or near the target nucleic acid in the genome of the cell, the second gRNA and the site-specific nuclease enzyme co-localize to a second genomic gRNA TS at or near the target nucleic acid in the genome of the cell, and the site-specific nuclease enzyme cleaves the genome of the cell in a site-specific manner.

74. The method of claim 71, wherein at least one gRNA, a second gRNA, and a third gRNA are present,
(a) wherein the at least one gRNA and the site-specific nuclease enzyme co-localize to the first gRNA TS of the cleavable ceDNA, the second gRNA and the site-specific nuclease enzyme co-localize to the second gRNA TS of the cleavable ceDNA, and the site-specific nuclease enzyme cleaves the cleavable ceDNA in a site-specific manner; and
wherein the third gRNA and the site-specific nuclease enzyme co-localize to at least a first genomic gRNA TS and/or a second genomic gRNA TS at or near the target nucleic acid in the genome of the cell, and the site-specific nuclease enzyme cleaves the genome of the cell in a site-specific manner, or
(b) wherein the at least one gRNA and the site-specific nuclease enzyme co-localize to the first gRNA TS of the cleavable ceDNA, the at least one gRNA and the site-specific nuclease enzyme co-localize to the second gRNA TS of the cleavable ceDNA, and the site-specific nuclease enzyme cleaves the cleavable ceDNA in a site-specific manner; and
wherein the second gRNA and the site-specific nuclease enzyme co-localize to a first genomic gRNA TS at or near the target nucleic acid in the genome of the cell, the third gRNA and the site-specific nuclease enzyme co-localize to a second genomic gRNA TS at or near the target nucleic acid in the genome of the cell, and the site-specific nuclease enzyme cleaves the genome of the cell in a site-specific manner.

75. The method of claim 71, wherein at least one gRNA, a second gRNA, a third gRNA, and a fourth gRNA are present,
wherein the at least one gRNA and the site-specific nuclease enzyme co-localize to the first gRNA TS of the cleavable ceDNA, the second gRNA and the site-specific nuclease enzyme co-localize to the second gRNA TS of the cleavable ceDNA, and the site-specific nuclease enzyme cleaves the cleavable ceDNA in a site-specific manner; and
wherein the third gRNA and the site-specific nuclease enzyme co-localize to a first genomic gRNA TS at or near the target nucleic acid in the genome of the cell, the fourth gRNA and the site-specific nuclease enzyme co-localize to a second genomic gRNA TS at or near the target nucleic acid in the genome of the cell, and the site-specific nuclease enzyme cleaves the genome of the cell in a site-specific manner.

76. The method of any one of claims 71-75, wherein the contacting effects non-homologous end joining (NHEJ), microhomology-mediated end-joining (MMEJ), or homology directed recombination (HDR) and editing of the target nucleic acid.

77. The method of any one of claims 71-76, wherein the cell is a eukaryotic cell.

78. The method of any one of claims 71-77, wherein the contacting is in vitro, ex vivo, or in vivo.

79. The method of any one of claims 71-78, wherein the method is performed in vivo to correct a single nucleotide polymorphism (SNP) associated with a disease.

80. The method of any of claims 71-79, further comprising administering the cells produced to a subject in need thereof.

81. The method of claim 80, wherein the subject in need thereof has a genetic disease, a viral infection, a bacterial infection, a parasitic infection, a fungal infection, a cancer, or an autoimmune disease.

82. A cell edited by the method of any one of claims 71-79.

83. A method of producing a cleaved ceDNA, the method comprising contacting the cleavable ceDNA of any one of claims 1-48 or the isolated polynucleotide of claim 49 with a site-specific nuclease enzyme and at least one guide RNA (gRNA), wherein the at least one gRNA and the site-specific nuclease enzyme co-localize to the first gRNA TS and/or the second gRNA TS of the cleavable ceDNA, and the site-specific nuclease enzyme cleaves the cleavable ceDNA in a site-specific manner, thereby producing the cleaved ceDNA.

84. The method of claim 83, further comprising purifying the cleaved ceDNA.

85. A cleaved ceDNA produced by the method of claim 83 or claim 84.

86. A cleavable non-viral capsid-free closed-ended DNA (ceDNA) comprising in the following order:
a first inverted terminal repeat (ITR);
a first guide RNA (gRNA) target sequence (TS) and a first protospacer adjacent motif (PAM);
at least one transgene cassette;
a second gRNA target sequence (TS) and a second protospacer adjacent motif (PAM); and
a second ITR.
